(19) United States
(12) Patent Application Publication
(10) Pub. No.: US 2024/0148883 A1
(43) Pub. Date: May 9, 2024

(54) Branched Linkers for Antibody-Drug Conjugates and Methods of Use Thereof

(71) Applicant: R.P. Scherer Technologies, LLC, Carson City, NV (US)

(72) Inventors: Stepan Chuprakov, Walnut Creek, CA (US); Ayodele O. Ogunkoya, Berkeley, CA (US); Penelope M. Drake, Castro Valley, CA (US)

(21) Appl. No.: 18/276,432

(22) PCT Filed: Mar. 2, 2022
(86) PCT No.: PCT/US2022/018534

§ 371 Date: Aug. 8, 2023

Related U.S. Application Data
(60) Provisional application No. 63/237,450, filed on Aug. 26, 2021.
(60) Provisional application No. 63/214,540, filed on Jun. 24, 2021.
(60) Provisional application No. 63/186,581, filed on May 10, 2021.
(60) Provisional application No. 63/156,156, filed on Mar. 3, 2021.

(51) Int. Cl. A61K 47/64; A61K 31/7056; A61K 31/706; A61P 35/00

(52) U.S. Cl. CPC A61K 47/64; A61K 31/7056; A61K 31/706; A61P 35/00

(57) ABSTRACT

The present disclosure provides antibody-drug conjugate structures. The antibody-drug conjugate structures include a branched linker where two or more payloads per branched linker are attached to a polypeptide (e.g., an antibody). In addition, the disclosure also encompasses compounds and methods for production of such conjugates. In addition, the disclosure also encompasses methods of using the conjugates.

### CROSS-REFERENCE TO RELATED APPLICATIONS

This application claim the benefit of priority to U.S. Application No. 63/237,450, filed Aug. 26, 2021, U.S. Application No. 63/156,156, filed Mar. 3, 2021, U.S. Application No. 63/186,581, filed May 10, 2021, and U.S. Application No. 63/214,540, filed Jun. 24, 2021, the disclosures of which are incorporated herein by reference.

### INTRODUCTION

Antibody-drug conjugates (ADCs) have emerged over the past two decades as a new class of targeted-delivery therapies. A typical ADC includes an antibody-based targeting element attached to a highly potent pharmaceutical agent (payload) via a chemical linker using an available bioconjugation method. The molar ratio of targeting element (e.g., antibody) to attached payload can vary, and is referred to as the drug-to-antibody ratio (DAR). Commonly used bioconjugation methods either exploit endogenous amino acid residues of a protein (i.e., lysine and cysteine), or rely on selective engagement of a bioorthogonal functional group that has been intentionally introduced into the protein. As an example of the latter approach, the Hydrazino-iso-Pictet-Spengler (HIPS) conjugation method (FIG. 1) takes an advantage of an aldehyde functional group (an “aldehyde tag”), which can be introduced into a protein, such as an antibody, through various means (e.g., by the action of formyl generating enzyme (FGE)), serving as the conjugation handle. The aldehyde group cleanly reacts with the HIPS indole moiety to form a stable carbon-carbon bond that permanently attaches the payload of choice to the protein in a single chemical step.

### SUMMARY

Traditionally, the HIPS conjugation method has been used to produce conjugates carrying one payload per HIPS moiety per aldehyde tag, which produces antibody conjugates with DAR values of up to 4. The present disclosure provides the use of branched HIPS linkers that carry two (or more) molecules of the same or different payload per one HIPS moiety and are therefore capable of conjugating two (or more) small molecule payloads per one aldehyde group in a protein in a single conjugation step (FIG. 2). Consequently, the usage of such branched linkers allows the generation of higher DAR site-specific conjugates (e.g., DAR up to 8) with controlled payload placement, which in the context of therapeutic ADCs would result in larger quantities of pharmaceutical agent delivered to the targeted tissue.

The present disclosure provides antibody-drug conjugate (ADC) structures, which include a branched HIPS linker. The disclosure also encompasses compounds and methods for production of such conjugates, as well as methods of using the conjugates.

Aspects of the present disclosure include a conjugate of formula (I):

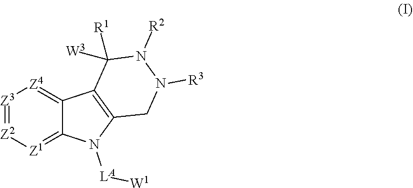

wherein:
   Z¹, Z², Z³ and Z⁴ are each independently selected from CR⁴, N and C-L^{B}-W², wherein at least one Z¹, Z², Z³ and Z⁴ is C-L^{B}-W²;
   R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl; R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
   each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
   L^{A} is a first linker;
   L^{B} is a second linker;
   W¹ is a first drug;
   W² is a second drug; and
   W³ is a polypeptide.

In some embodiments, Z¹ is CR⁴.

In some embodiments, Z¹ is N.

In some embodiments, Z³ is C-L^{B}-W².

In some embodiments, L^{A} comprises:

-(T¹-V¹)ₐ-(T²-V²)_{b}-(T³-V³)_{c}-(T⁴-V⁴)_{d}-(T⁵-V⁵)ₑ-(T⁶-V⁶)_{f}—,
wherein
   a, b, c, d, e and f are each independently 0 or 1;
   T¹, T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
   V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
   each R¹³ is independently selected from hydrogen, an alkyl, a substituted alkyl, an aryl, and a substituted aryl; and
   each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

In some embodiments of L^{A}:
T¹ is selected from a (C₁-C₁₂)alkyl and a substituted (C₁-C₁₂)alkyl;
T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PHP, an acetal group, a hydrazine, and an ester; and
V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂—, and —P(O)OH—;
wherein:
(PEG)ₙ is

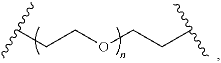

where n is an integer from 1 to 30;
EDA is an ethylene diamine moiety having the following structure:

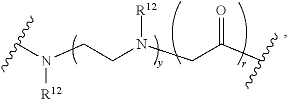

where y is an integer from 1 to 6 and r is 0 or 1;
4-amino-piperidine (4AP) is

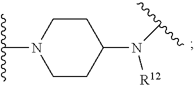

and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring.

In some embodiments, T¹, T², T³, T⁴, T⁵ and T⁶ are each optionally substituted with a glycoside.

In some embodiments, MABO, MABC, PABO, PABC, PAB, PABA, PAP and PHP are each optionally substituted with a glycoside.

In some embodiments, the glycoside is selected from a glucuronide, a galactoside, a glucoside, a mannoside, a fucoside, O-GlcNAc, and O-GalNAc.

In some embodiments, L^{A} is a linker wherein:
T¹ is (C₁-C₁₂)alkyl and V¹ is —CO—;
T² is AA and V² is absent;
T³ is PABC and V³ is absent; and
d, e and f are each 0; or
wherein:
T¹ is (C₁-C₁₂)alkyl and V¹ is —CONH—;
T² is (PEG)ₙ and V² is —CO—;
T³ is AA and V³ is absent;
T⁴ is PABC and V⁴ is absent; and
e and f are each 0; or
wherein:
T¹ is (C₁-C₁₂)alkyl and V¹ is —CO—;
T² is an amino acid analog and V² is —NH—;
T³ is (PEG)ₙ and V³ is —CO—;
T⁴ is AA and V⁴ is absent;
T⁵ is PABC and V⁵ is absent; and
f is 0; or
wherein:
T¹ is (C₁-C₁₂)alkyl and V¹ is —CONH—;
T² is (PEG)ₙ and V² is —CO—;
T³ is AA and V³ is absent;
T⁴ is PABC and V⁴ is absent; and
e and f are each 0; or
wherein:
T¹ is (C₁-C₁₂)alkyl and V¹ is —CONH—;
T² is substituted (C₁-C₁₂)alkyl and V² is —CO—;
T³ is AA and V³ is absent;
T⁴ is PABC and V⁴ is absent; and
e and f are each 0; or
wherein:
T¹ is (C₁-C₁₂)alkyl and V¹ is —CONH—;
T² is (PEG)ₙ and V² is —CO—;
T³ is AA and V³ is absent;
T⁴ is PABA and V⁴ is —CO—;
T⁵ is (C₁-C₁₂)alkyl and V⁵ is absent; and
f is 0; or
wherein:
T¹ is (C₁-C₁₂)alkyl and V¹ is —CO—;
T² is 4AP and V² is —CO—;
T³ is (C₁-C₁₂)alkyl and V³ is —CO—;
T⁴ is AA and V⁴ is absent;
T⁵ is PABC and V⁵ is absent; and
f is 0; or
wherein:
T¹ is (C₁-C₁₂)alkyl and V¹ is —CO—;
T² is 4AP and V² is —CO—;
T³ is (C₁-C₁₂)alkyl and V³ is —O—;
T⁴ is (C₁-C₁₂)alkyl and V⁴ is —CO—;
T⁵ is AA and V⁵ is absent; and
T⁶ is PABC and V⁶ is absent; or
wherein:
T¹ is (C₁-C₁₂)alkyl and V¹ is —CO—;
T² is an amino acid analog and V² is absent;
T³ is AA and V³ is absent;
T⁴ is PABC and V⁴ is absent; and
e and f are each 0; or
wherein:
T¹ is (C₁-C₁₂)alkyl and V¹ is —CONH—;
T² is (PEG)ₙ and V² is —CONH—;
T³ is substituted (C₁-C₁₂)alkyl and V³ is —CO—;
T⁴ is AA and V⁴ is absent;
T⁵ is PABC and V⁵ is absent; and
f is 0; or
wherein:
T¹ is (C₁-C₁₂)alkyl and V¹ is —CO—;
T² is an AA and V² is —NH—;
T³ is (PEG)ₙ and V³ is —CO—;
T⁴ is AA and V⁴ is absent;
T⁵ is PABC and V⁵ is absent; and
f is 0; or
wherein:
T¹ is (C₁-C₁₂)alkyl and V¹ is —CONH—;
T² is (PEG)ₙ and V² is —CO—;
T³ is AA and V³ is absent;
T⁴ is PAP and V⁴ is —C(O)O—; and
e and f are each 0; or
wherein:
T¹ is (C₁-C₁₂)alkyl and V¹ is —CONH—;
T² is substituted (C₁-C₁₂)alkyl and V² is —CO—;
T³ is AA and V³ is absent;
T⁴ is PABC and V⁴ is absent; and
e and f are each 0; or
wherein:
T¹ is (C₁-C₁₂)alkyl and V¹ is —CONH—;
T² is substituted (C₁-C₁₂)alkyl and V² is —CO—;
T³ is PABC and V³ is absent; and
d, e and f are each 0.

In some embodiments, L^{B} comprises:

-(T⁷-V⁷)_{g}-(T⁸-V⁸)ₕ-(T⁹-V⁹)ᵢ-(T¹⁰-V¹⁰)ⱼ-(T¹¹-V¹¹)ₖ-(T¹²-V¹²)ₗ-(T¹³V¹³)ₘ,
wherein
   g, h, i, j, k, l and m are each independently 0 or 1;
   T⁷, T⁸, T⁹, T¹⁰, T¹¹, T¹² and T¹³ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
   V⁷, V⁸, V⁹, V¹⁰, V¹¹, V¹² and V¹³ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
   each R¹³ is independently selected from hydrogen, an alkyl, a substituted alkyl, an aryl, and a substituted aryl; and
   each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

In some embodiments, T⁷, T⁸, T⁹, T¹⁰, T¹¹, T¹² and T¹³ are each optionally substituted with a glycoside.

In some embodiments, MABO, MABC, PABO, PABC, PAB, PABA, PAP and PHP are each optionally substituted with a glycoside.

In some embodiments, the glycoside is selected from a glucuronide, a galactoside, a glucoside, a mannoside, a fucoside, O-GlcNAc, and O-GalNAc.

In some embodiments, L^{B} is a linker wherein:
T⁷ is absent and V⁷ is —NHCO—;
T⁸ is (C₁-C₁₂)alkyl and V⁸ is —CO—;
T⁹ is AA and V⁹ is absent;
T¹⁰ is PABC and V¹⁰ is absent; and
k, l and m are each 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is AA and V¹⁰ is absent; and
T¹¹ is PABC and V¹¹ is absent; and
l and m are each 0; or
wherein:
T⁷ is absent and V⁷ is —NHCO—;
T⁸ is (C₁-C₁₂)alkyl and V⁸ is —CO—;
T⁹ is an amino acid analog and V⁹ is —NH—;
T¹⁰ is (PEG)ₙ and V¹⁰ is —CO—;
T¹¹ is AA and V¹¹ is absent;
T¹² is PABC and V¹² is absent; and
m is 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is AA and V¹⁰ is absent;
T¹¹ is PABC and V¹¹ is absent; and
l and m are each 0; or
wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is AA and V¹⁰ is absent;
T¹¹ is PABC and V¹¹ is absent; and
l and m are each 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is AA and V¹⁰ is absent;
T¹¹ is PABA and V¹¹ is —CO—;
T¹² is (C₁-C₁₂)alkyl and V¹² is absent; and
m is 0; or
wherein:
T⁷ is absent and V⁷ is —NHCO—;
T⁸ is (C₁-C₁₂)alkyl and V⁸ is —CO—;
T⁹ is 4AP and V⁹ is —CO—;
T¹⁰ is (C₁-C₁₂)alkyl and V¹⁰ is —CO—;
T¹¹ is AA and V¹¹ is absent;
T¹² is PABC and V¹² is absent; and
m is 0; or
wherein:
T⁷ is absent and V⁷ is —NHCO—;
T⁸ is (C₁-C₁₂)alkyl and V⁸ is —CO—;
T⁹ is 4AP and V⁹ is —CO—;
T¹⁰ is (C₁-C₁₂)alkyl and V¹⁰ is —O—;
T¹¹ is (C₁-C₁₂)alkyl and V¹¹ is —CO—;
T¹² is AA and V¹² is absent; and
T¹³ PABC and V¹³ is absent; or
wherein:
T⁷ is absent and V⁷ is —NHCO—;
T⁸ is (C₁-C₁₂)alkyl and V⁸ is —CO—;
T⁹ is an amino acid analog and V⁹ is absent;
T¹⁰ is AA and V¹⁰ is absent;
T¹¹ is PABC and V¹¹ is absent; and
l and m are each 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CONH—;
T¹⁰ is substituted (C₁-C₁₂)alkyl and V¹⁰ is —CO—;
T¹¹ is AA and V¹¹ is absent;
T¹² is PABC and V¹² is absent; and
m is 0; or
wherein:
T⁷ is absent and V⁷ is —NHCO—;
T⁸ is (C₁-C₁₂)alkyl and V⁸ is —CO—;
T⁹ is AA and V⁹ is —NH—;
T¹⁰ is (PEG)ₙ and V¹⁰ is —CO—;
T¹¹ is AA and V¹¹ is absent;
T¹² is PABC and V¹² is absent; and
m is 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is AA and V¹⁰ is absent;
T¹¹ is PAP and V¹¹ is —C(O)O—; and
l and m are each 0; or
wherein:
T⁷ is absent and V⁷ is —NHCO—;
T⁸ is (C₁-C₁₂)alkyl and V⁸ is —CO—;
T⁹ is AA and V⁹ is absent;
T¹⁰ is PABC and V¹⁰ is absent;
T¹¹ is PAP and V¹¹ is —C(O)O—; and
l and m are each 0; or
wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is PABC and V¹⁰ is absent; and
k, l and m are each 0; or
wherein:
T⁷ is absent and V⁷ is —NHCO—;
T⁸ is (C₁-C₁₂)alkyl and V⁸ is absent;
T⁹ is heteroaryl and V⁹ is absent;
T¹⁰ is (C₁-C₁₂)alkyl and V¹⁰ is —CONH—;
T¹¹ is (PEG)ₙ and V¹¹ is —CO—; and
l and m are each 0; or
wherein:
T⁷ is absent and V⁷ is —NHCO—;
T⁸ is (C₁-C₁₂)alkyl and V⁸ is absent;
T⁹ is heteroaryl and V⁹ is absent;
T¹⁰ is (C₁-C₁₂)alkyl and V¹⁰ is —CONH—;
T¹¹ is substituted (C₁-C₁₂)alkyl and V¹¹ is —CO—;
T¹² is AA and V¹² is absent; and
T¹³ PAB and V¹³ is absent; or
wherein:
T⁷ is absent and V⁷ is —NHCO—;
T⁸ is (C₁-C₁₂)alkyl and V⁸ is absent;
T⁹ is heteroaryl and V⁹ is absent;
T¹⁰ is (C₁-C₁₂)alkyl and V¹⁰ is —CONH—;
T¹¹ is substituted (C₁-C₁₂)alkyl and V¹¹ is —CO—;
T¹² is AA and V¹² is absent; and
T¹³ PABC and V¹³ is absent.

In some embodiments, the conjugate is selected from:

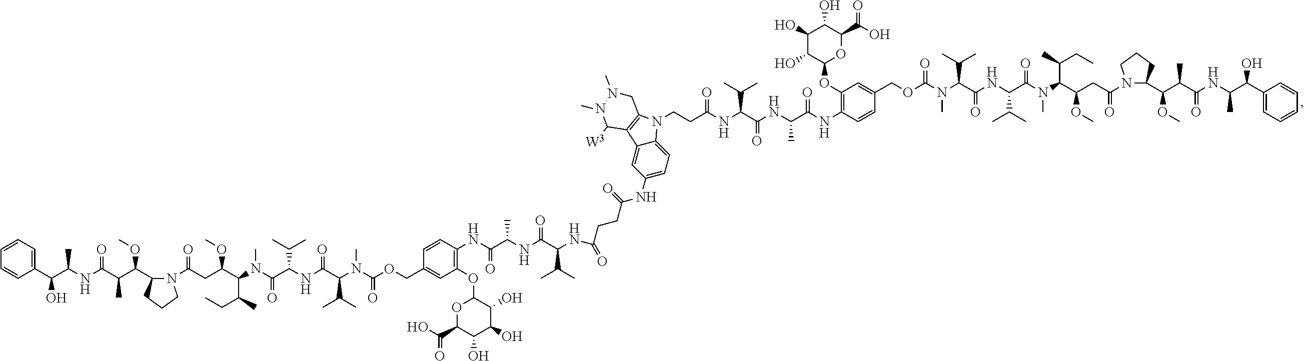

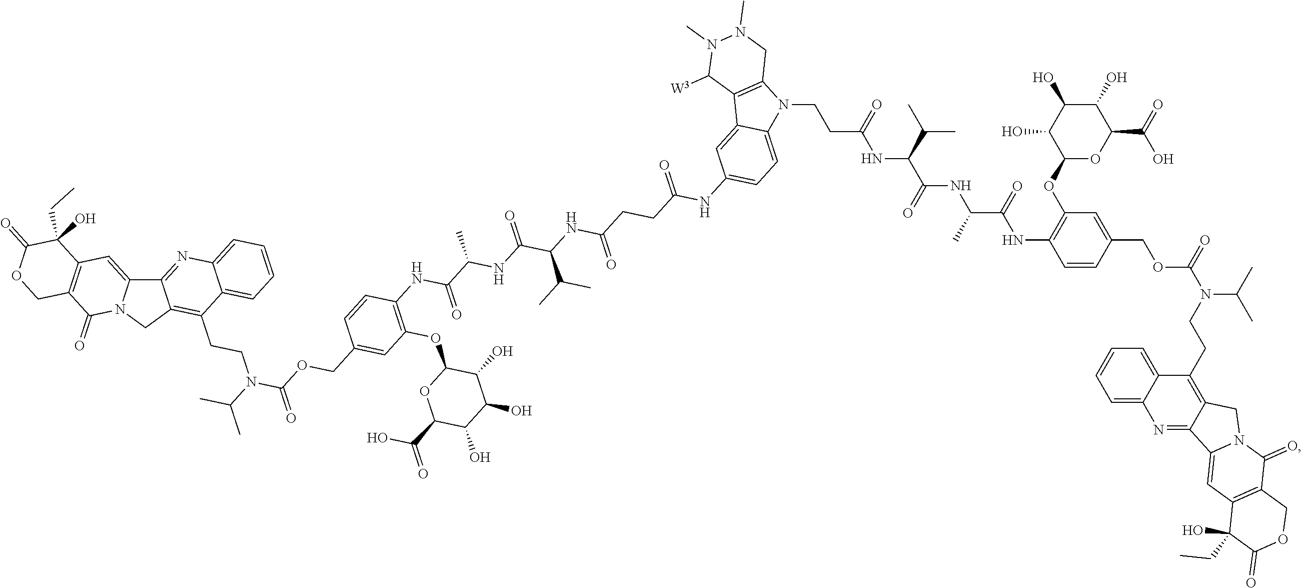

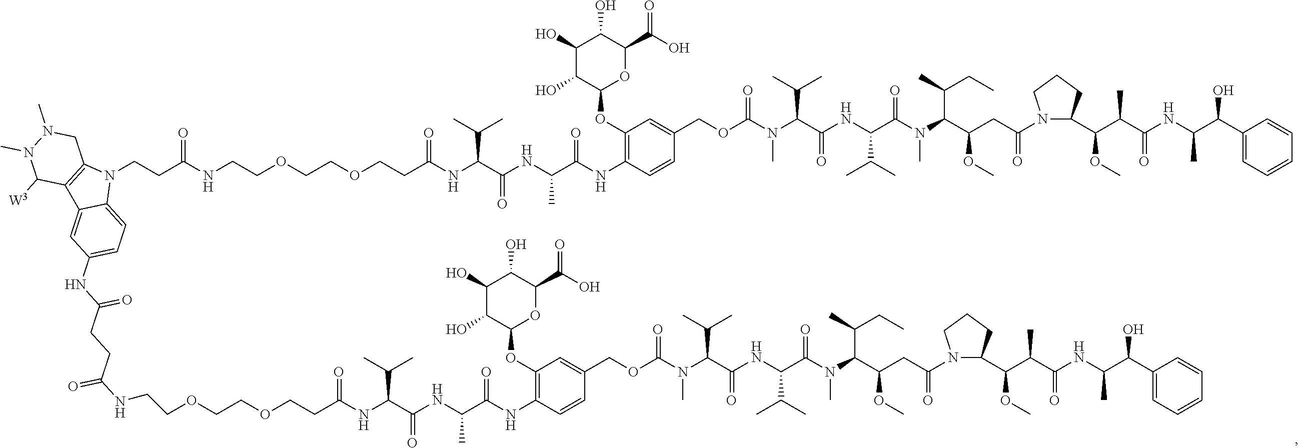

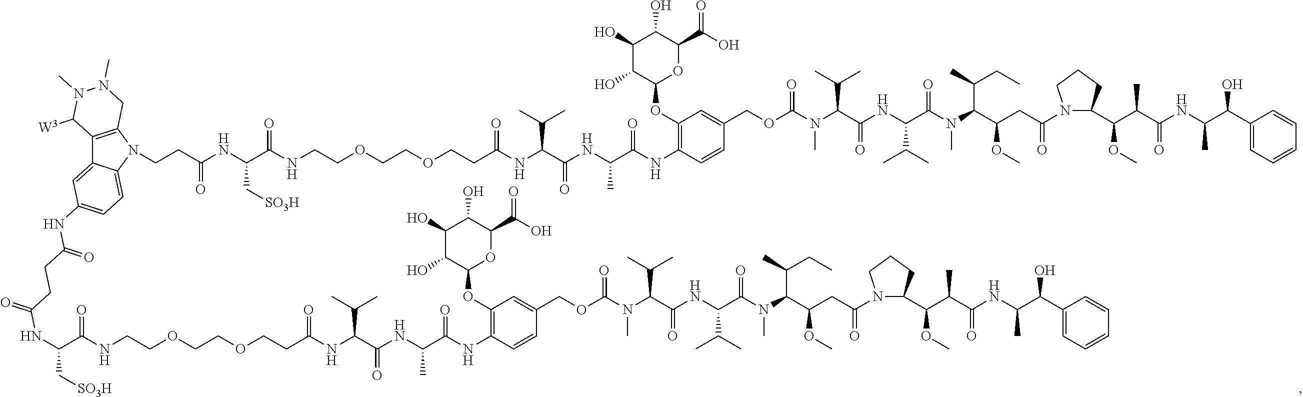

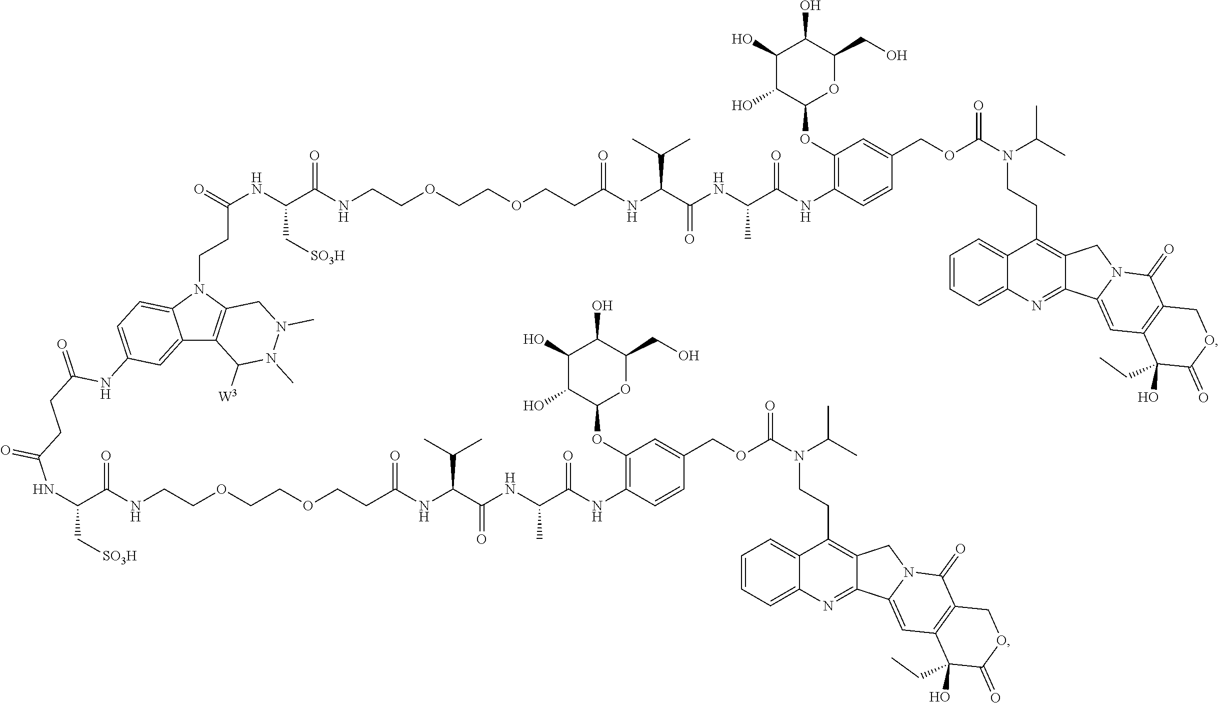

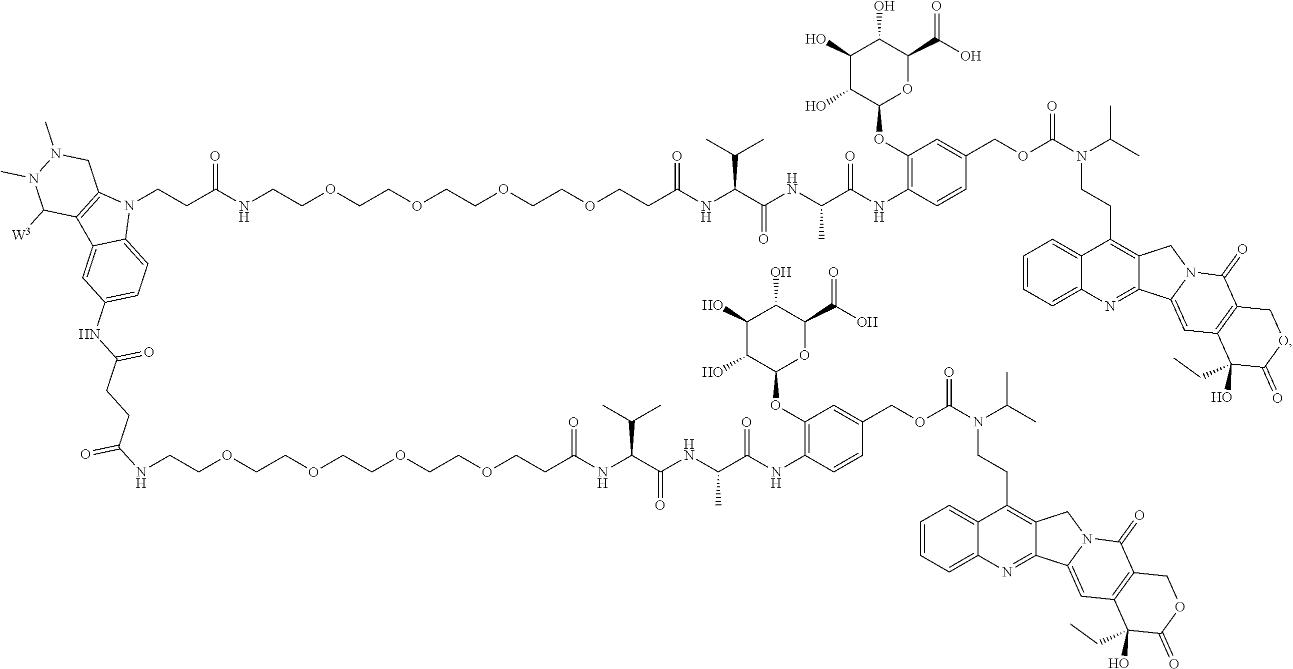

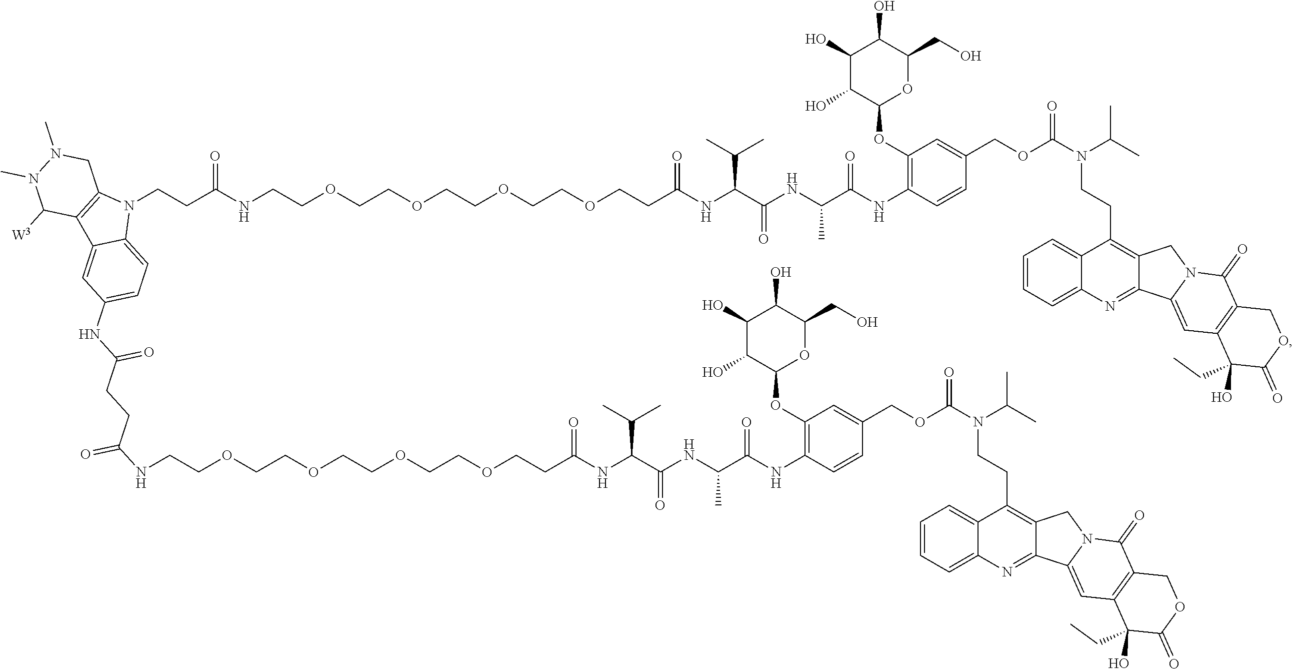

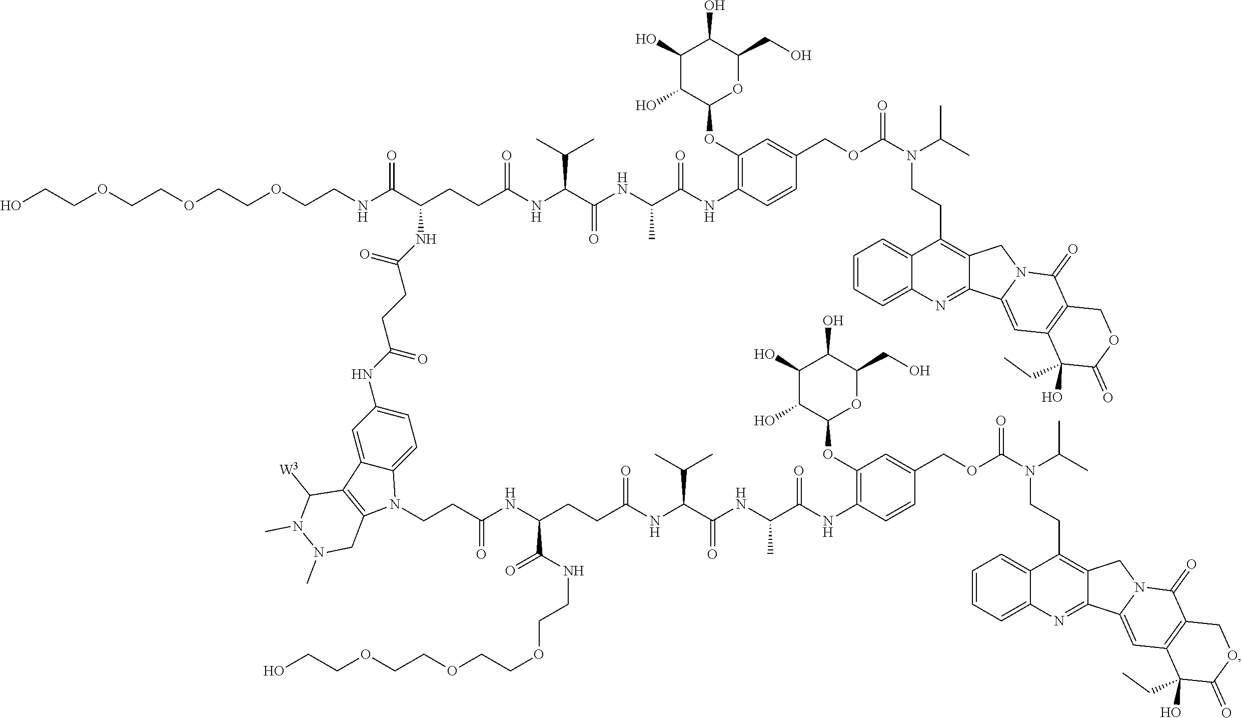

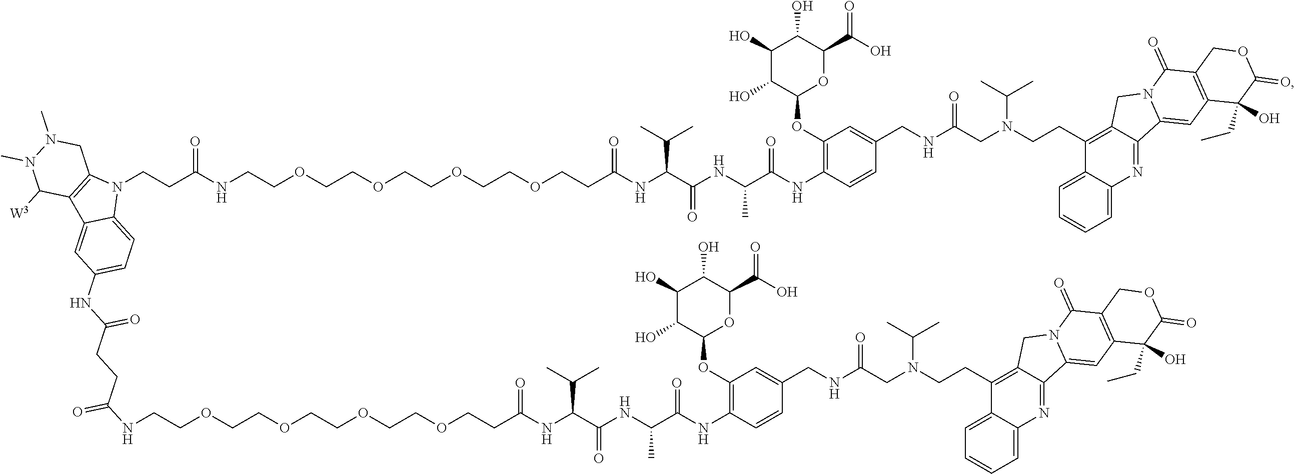

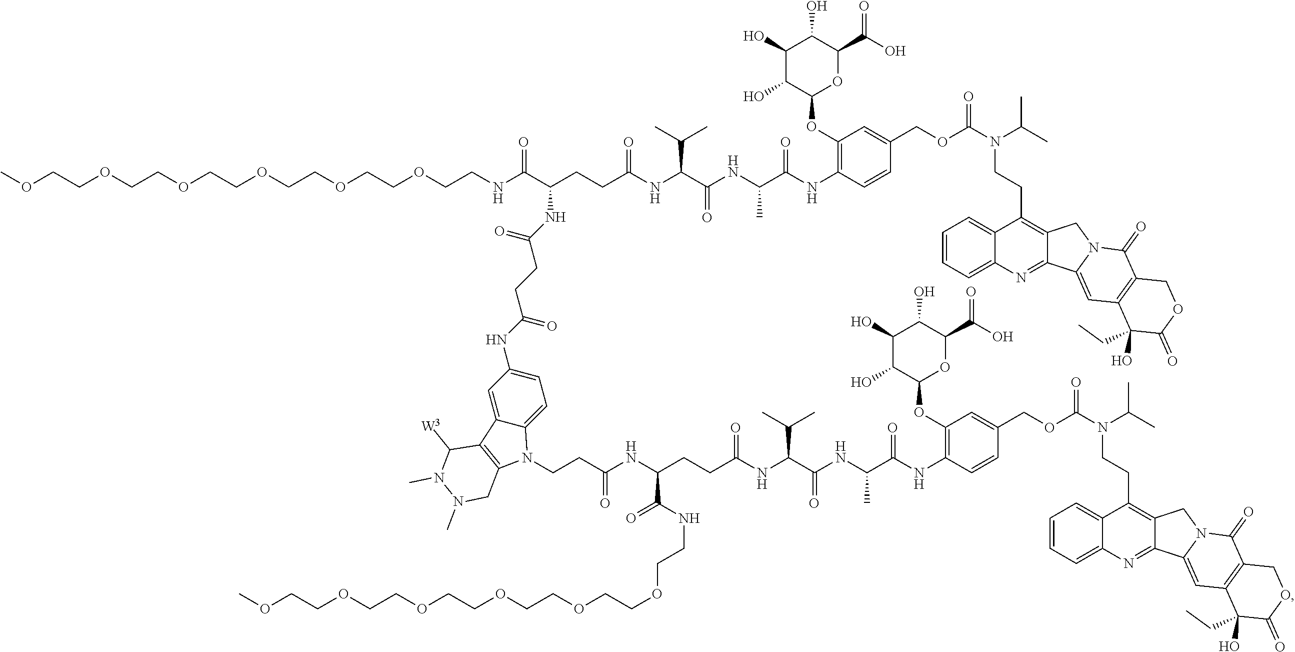

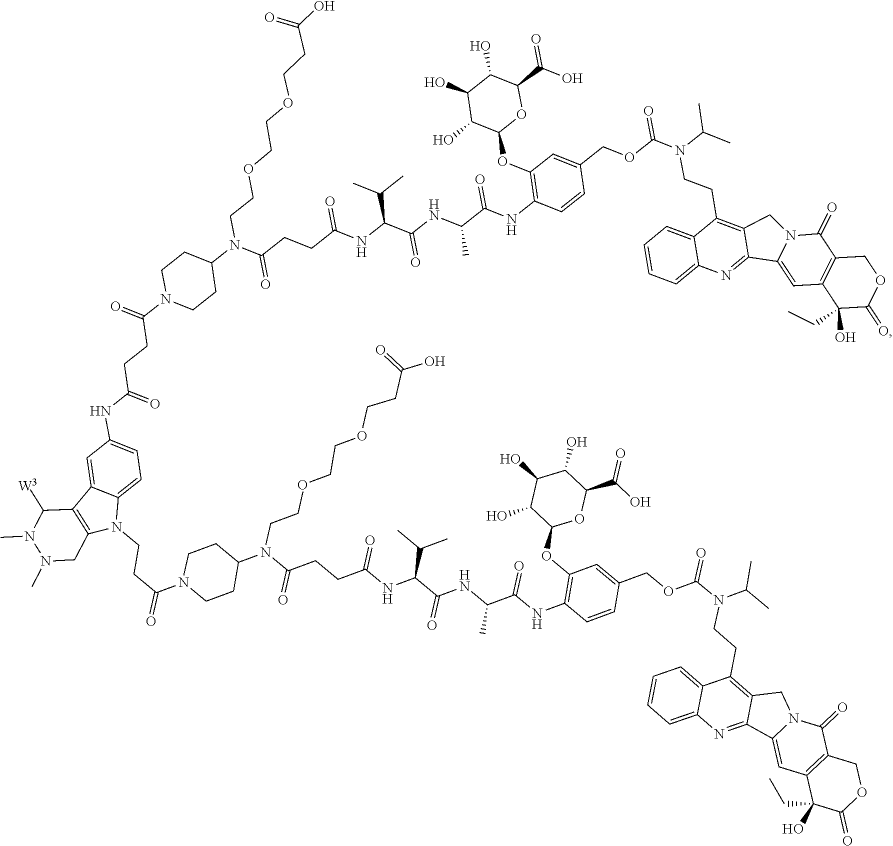

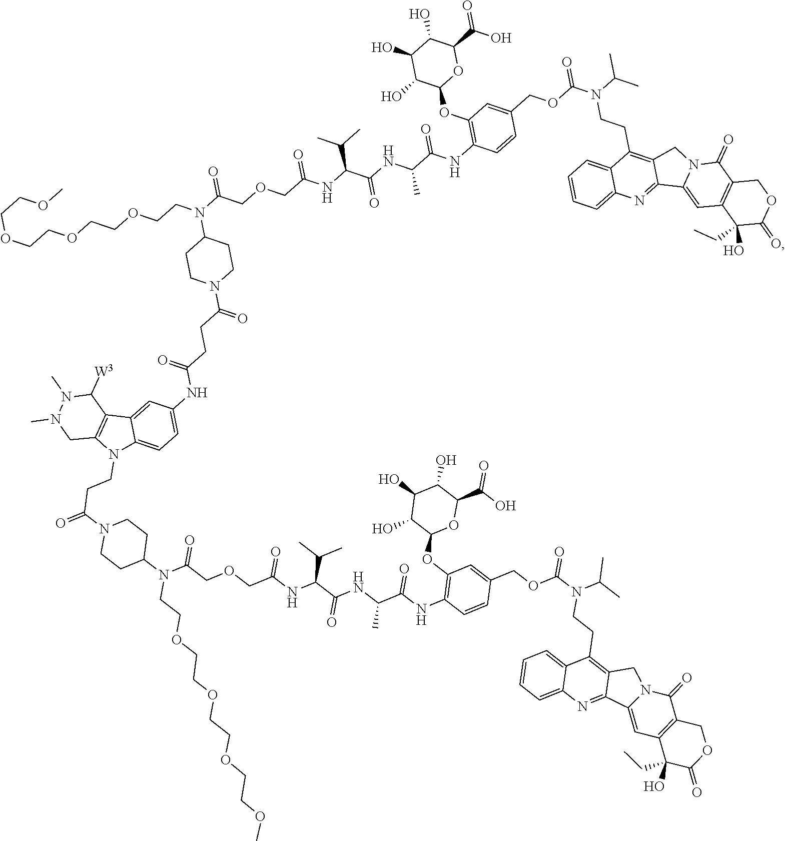

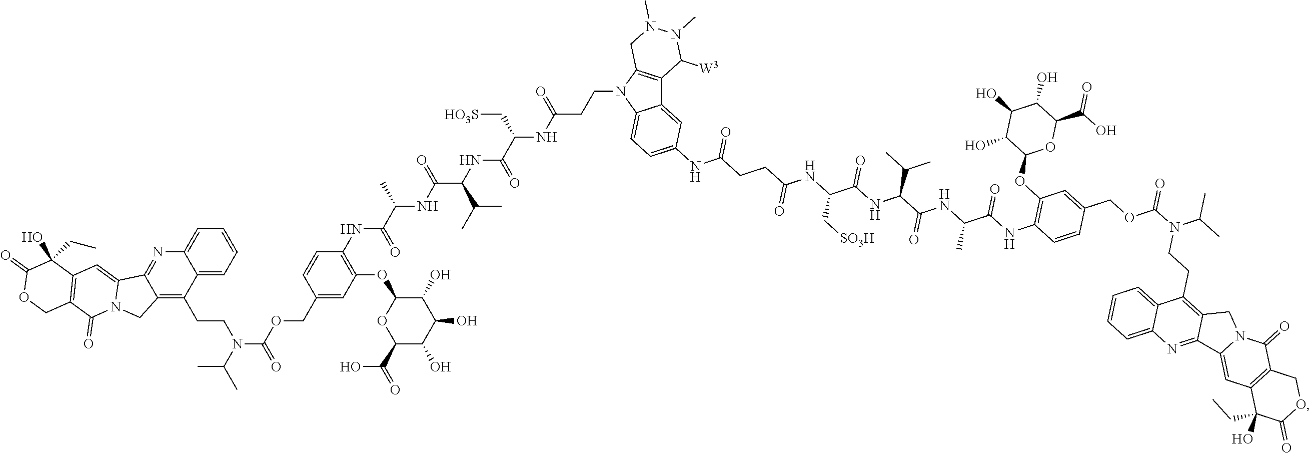

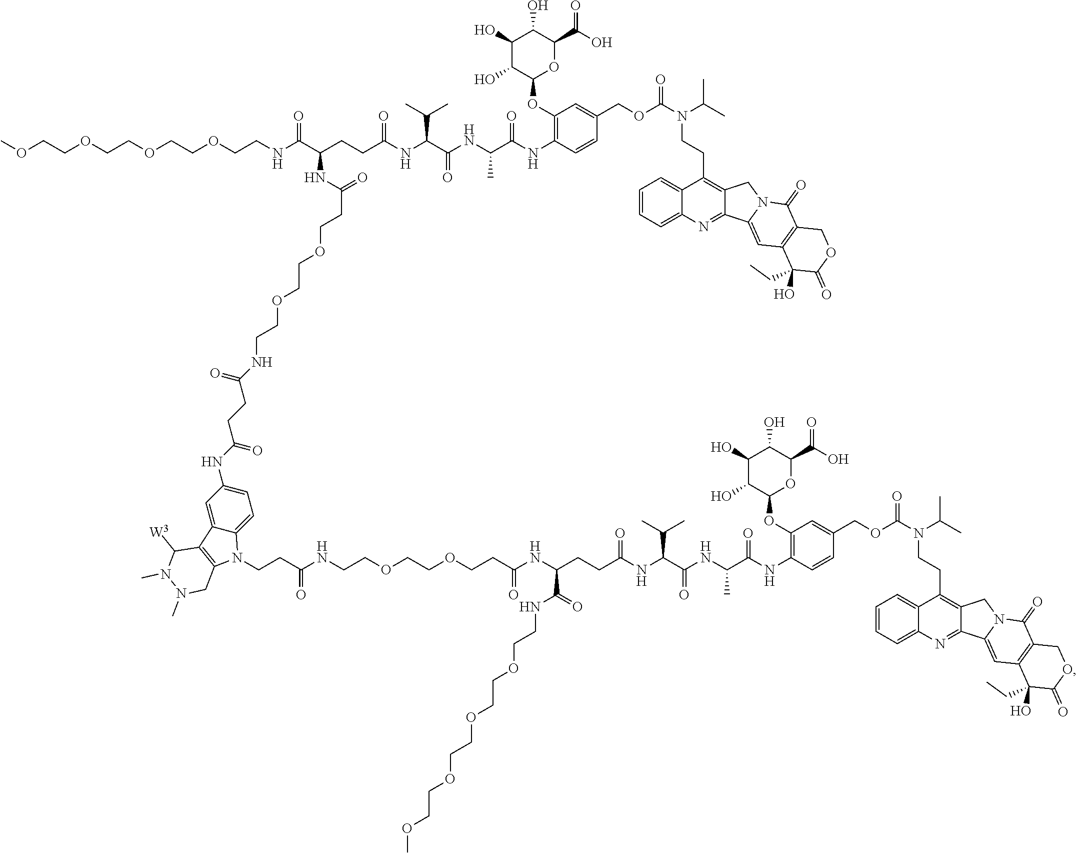

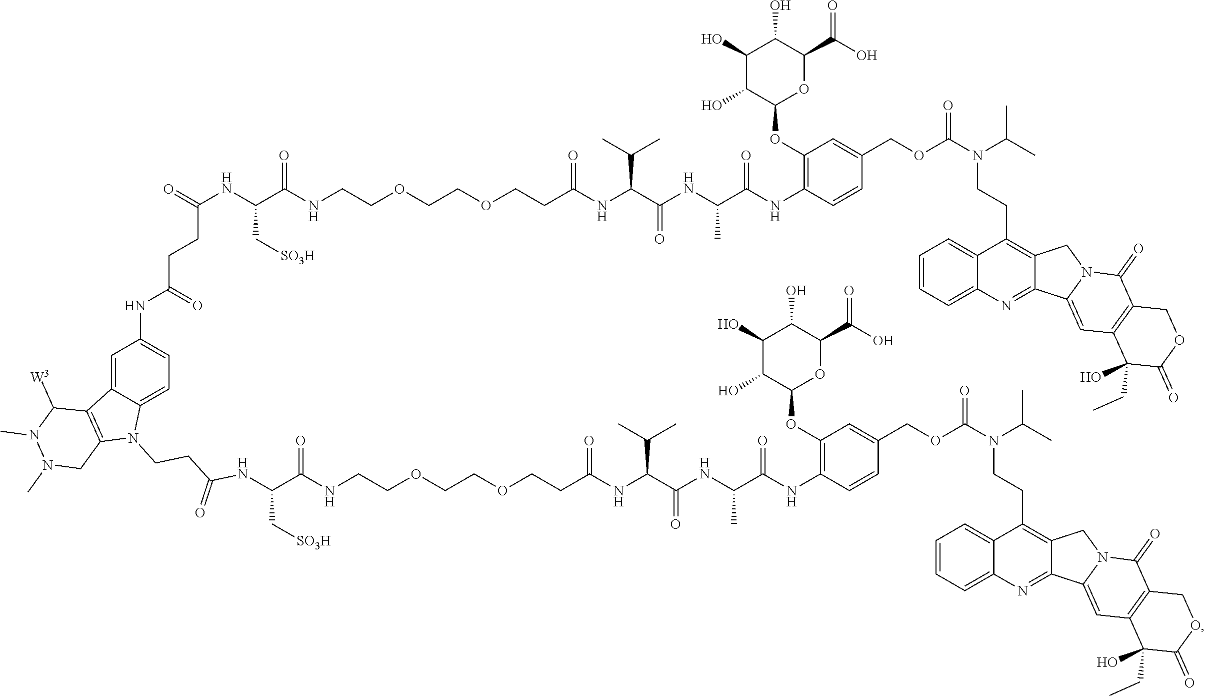

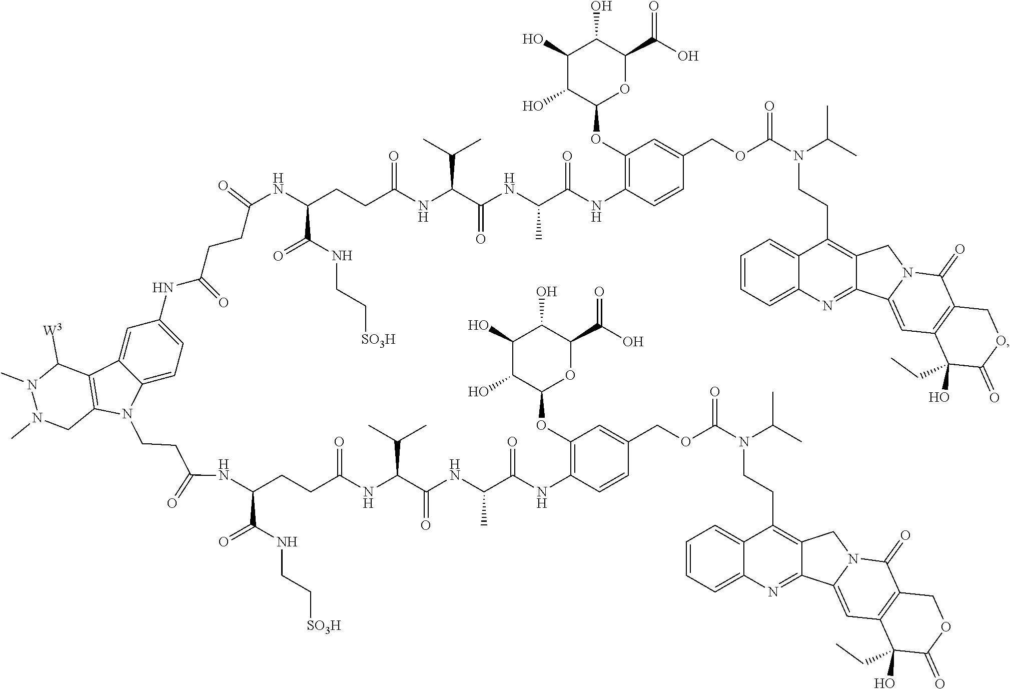

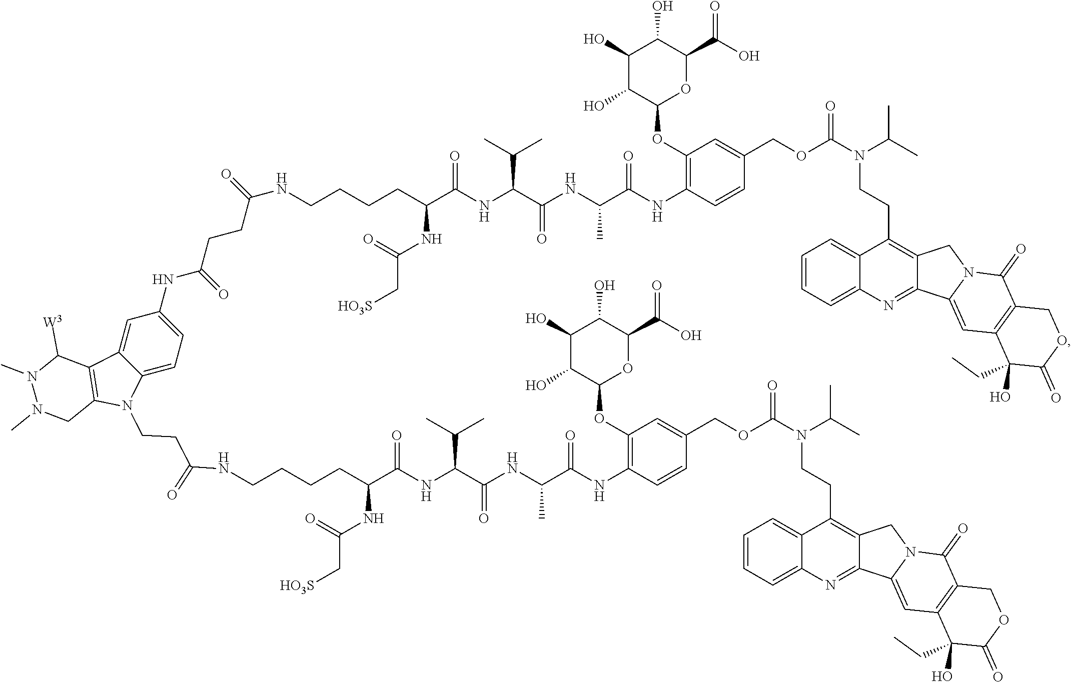

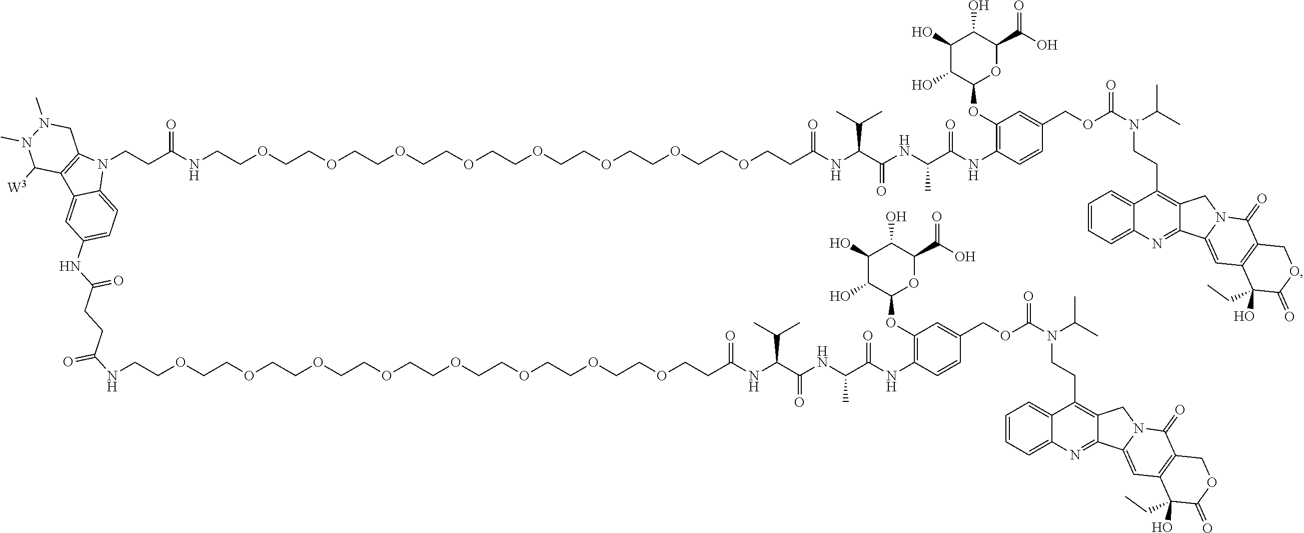

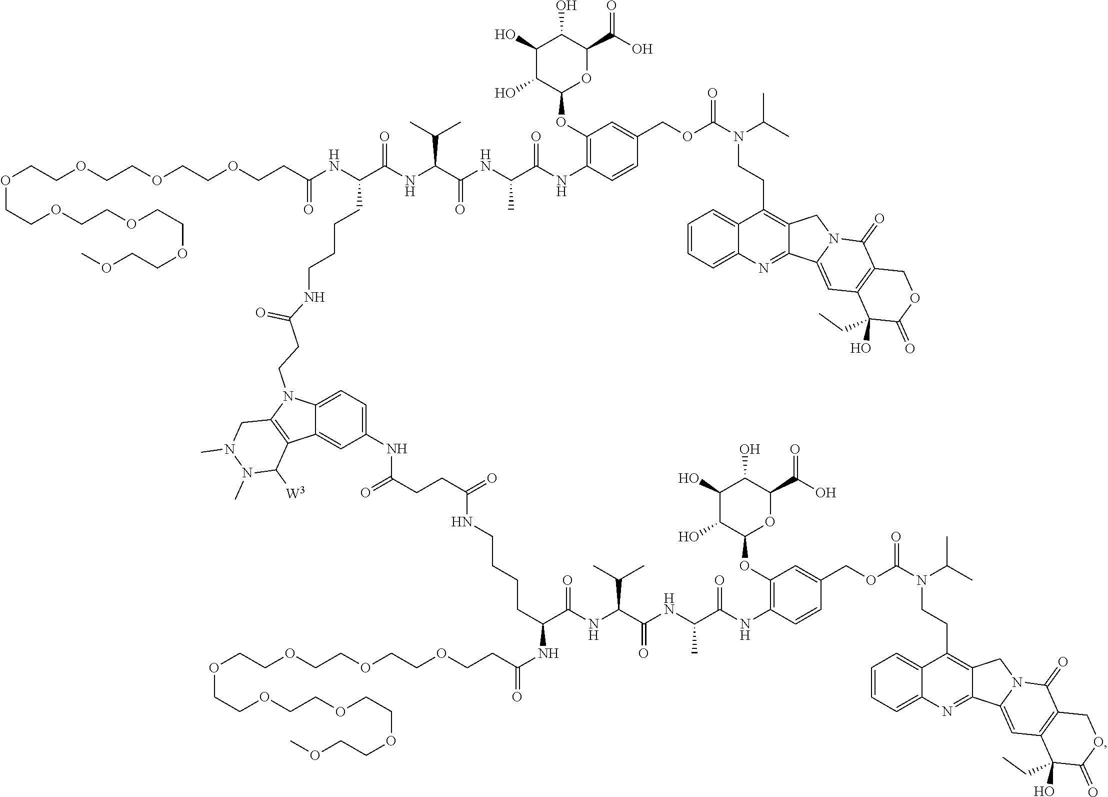

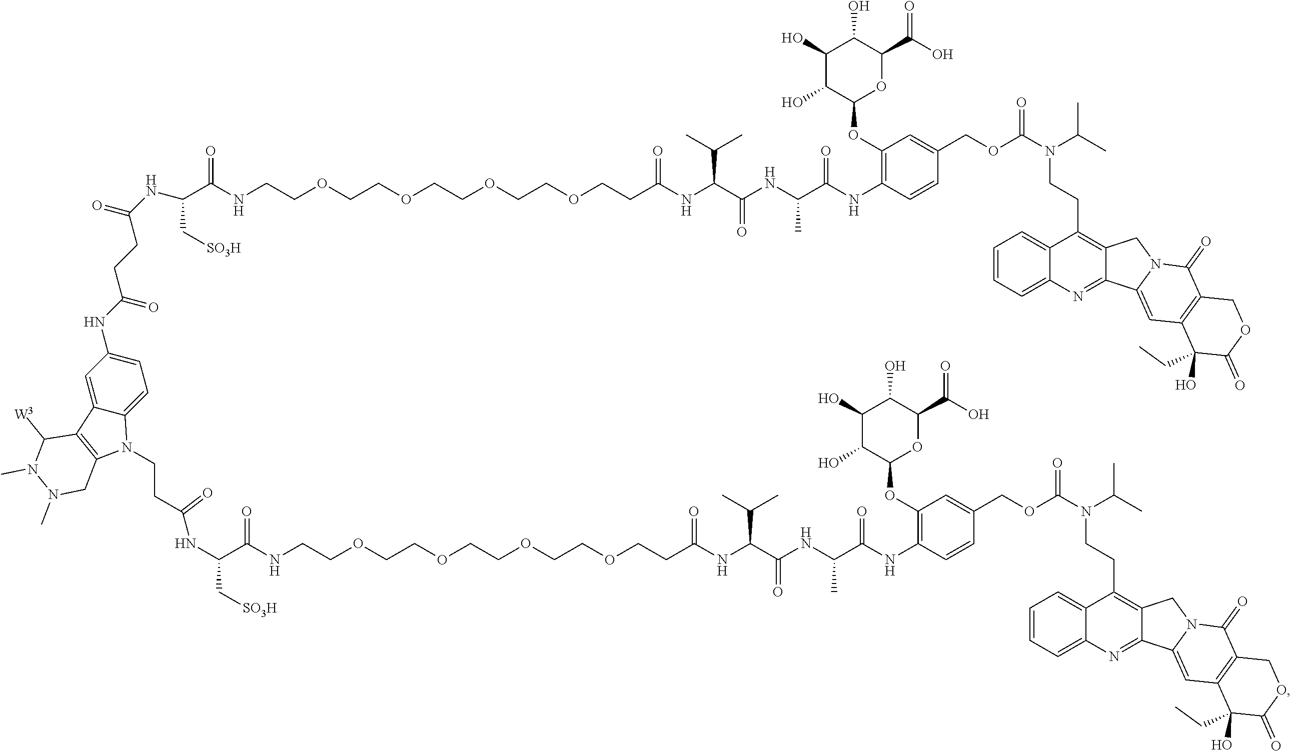

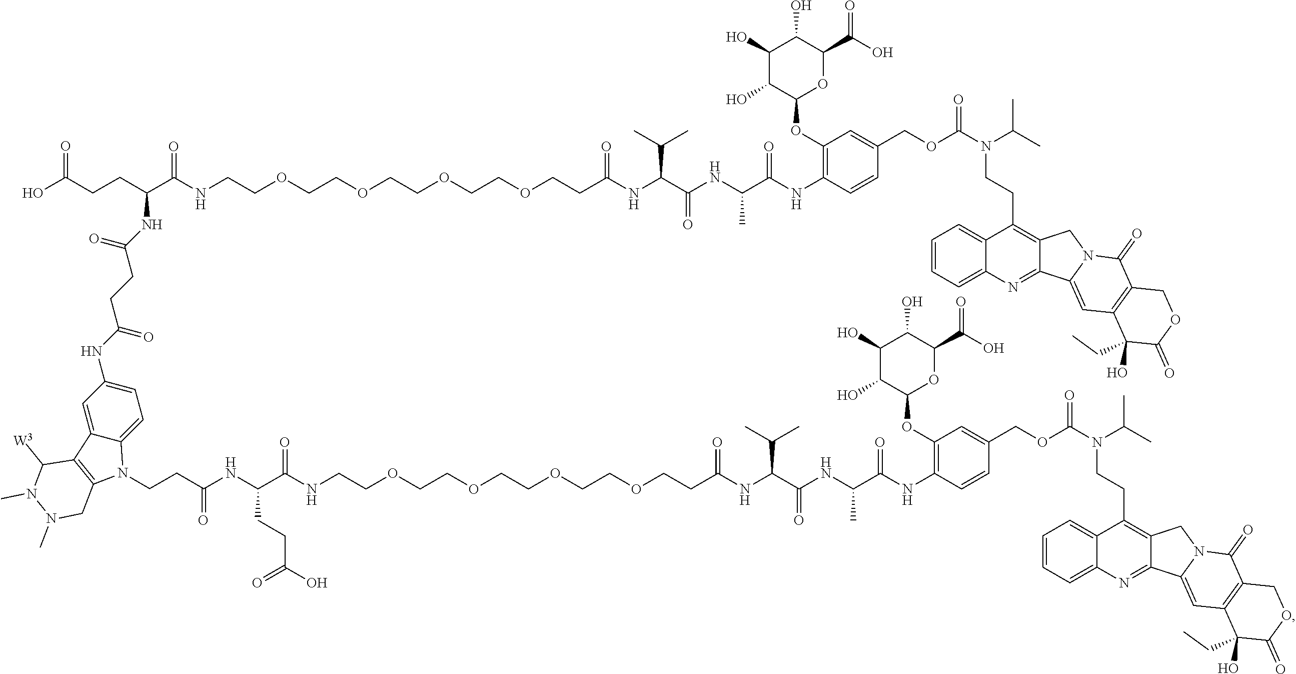

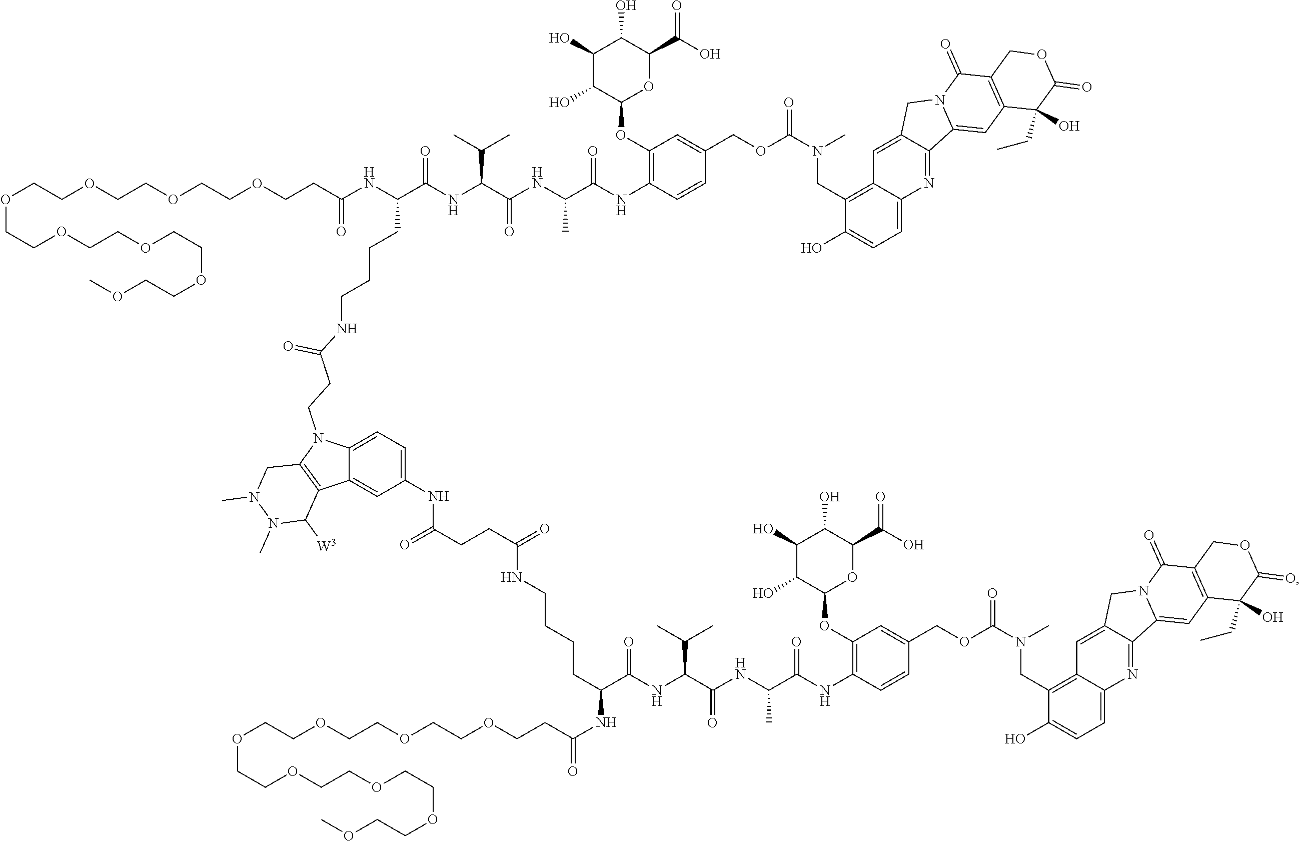

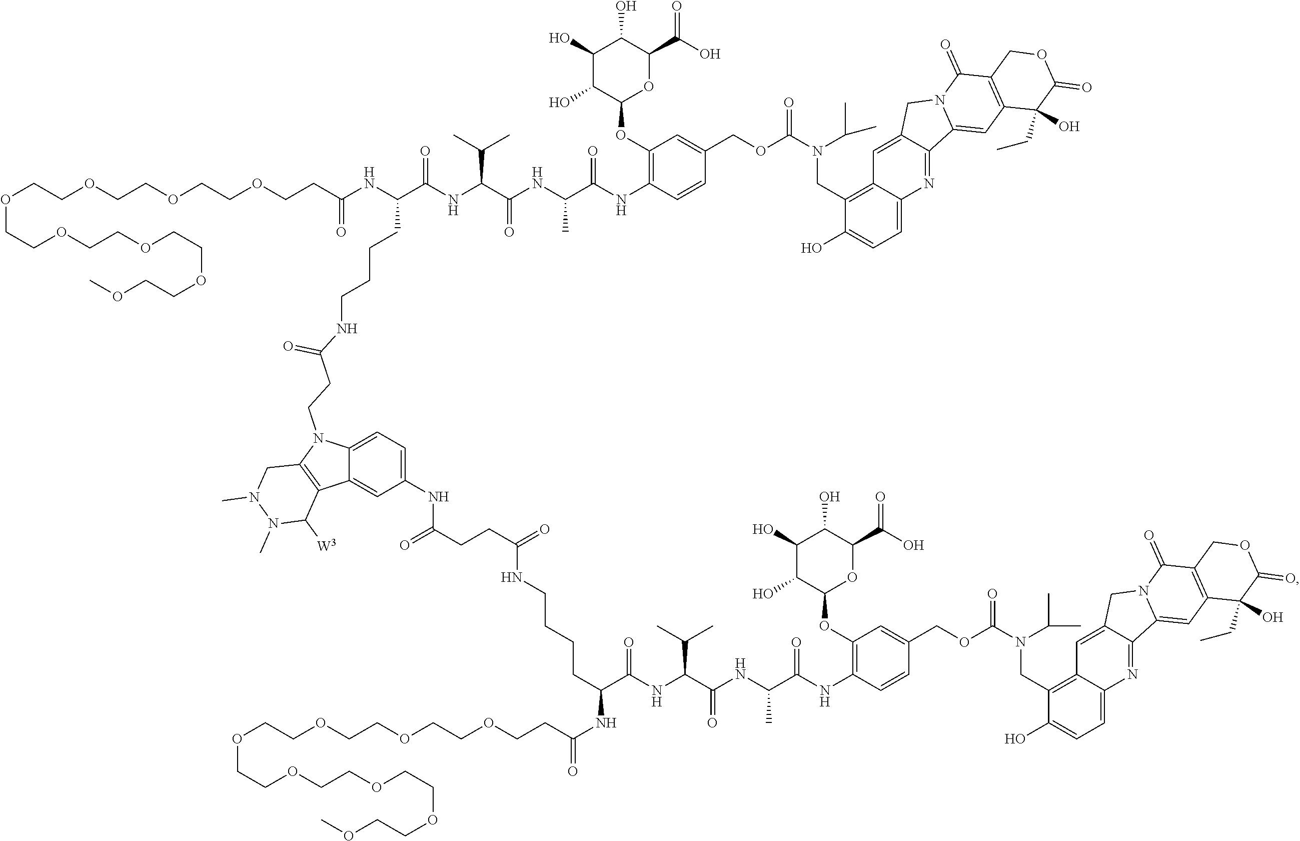

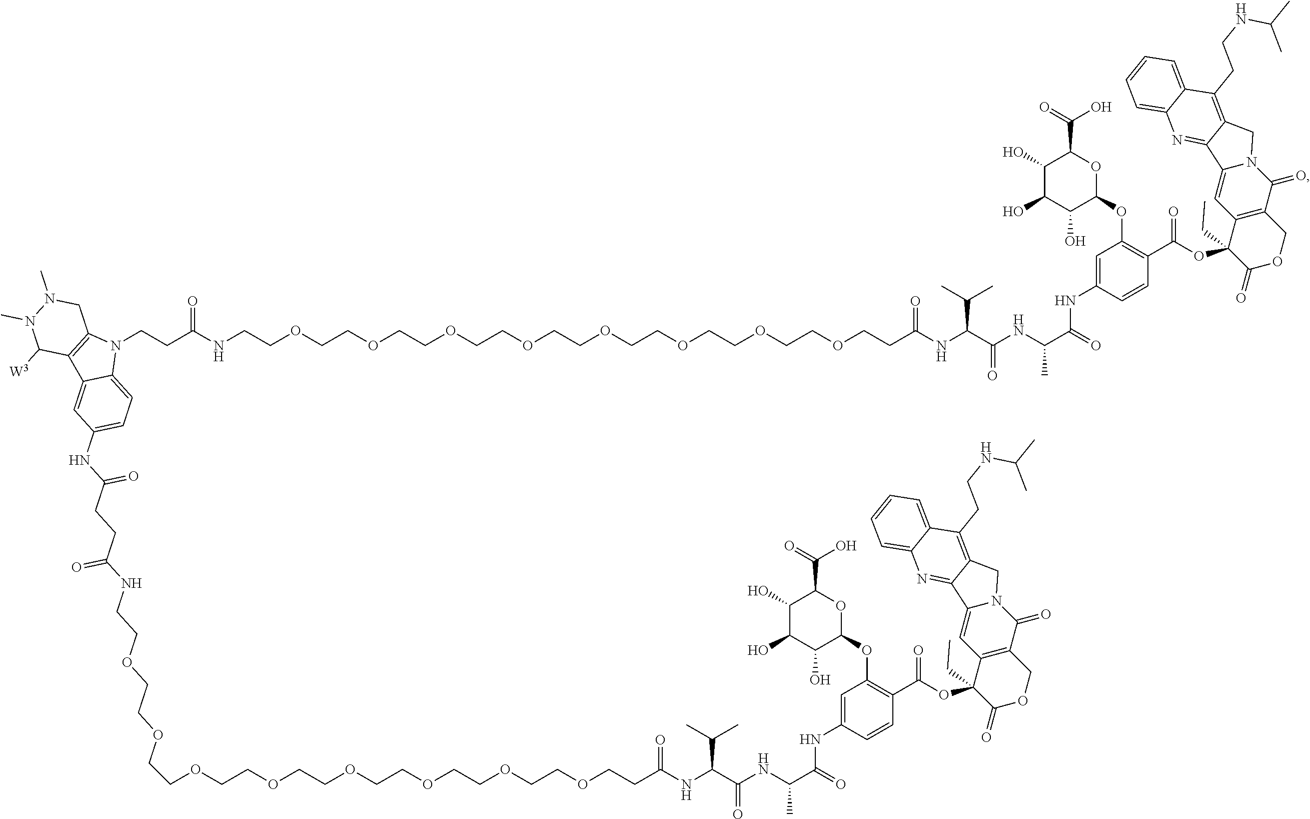

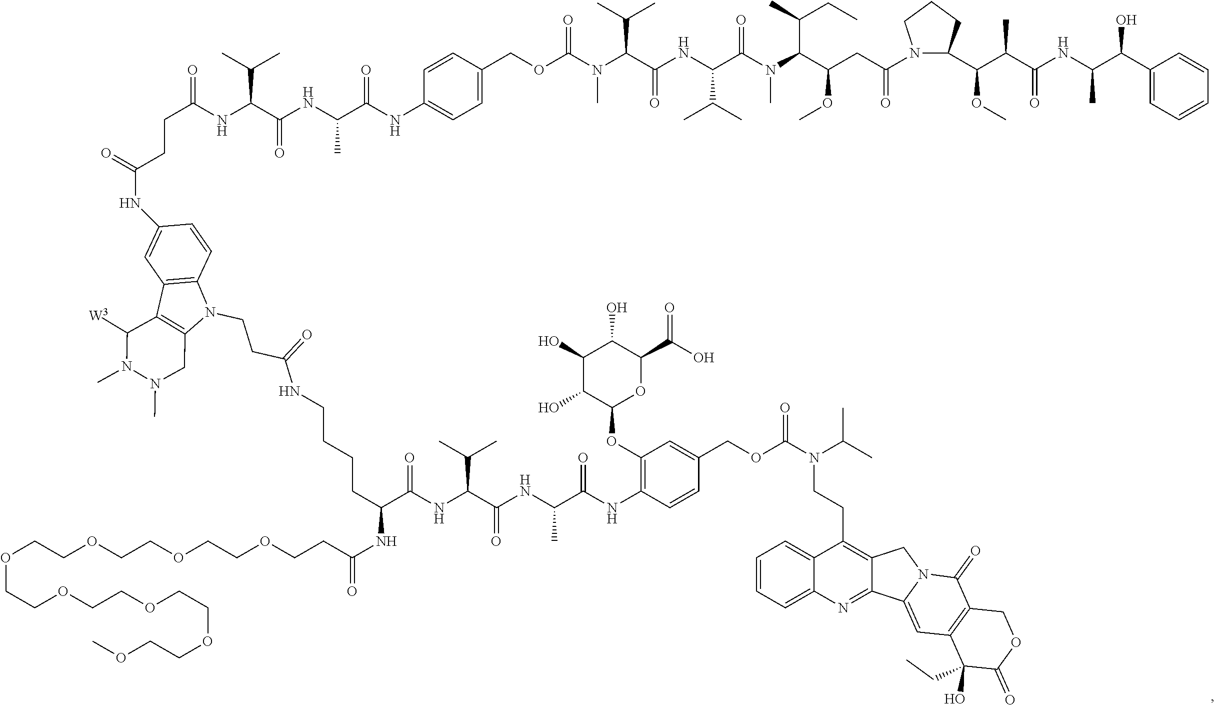

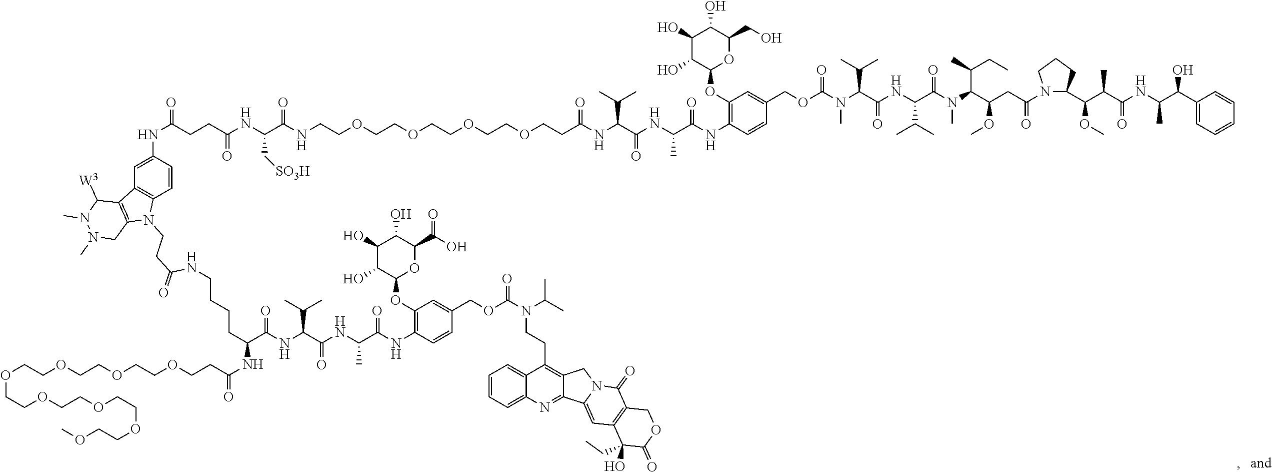

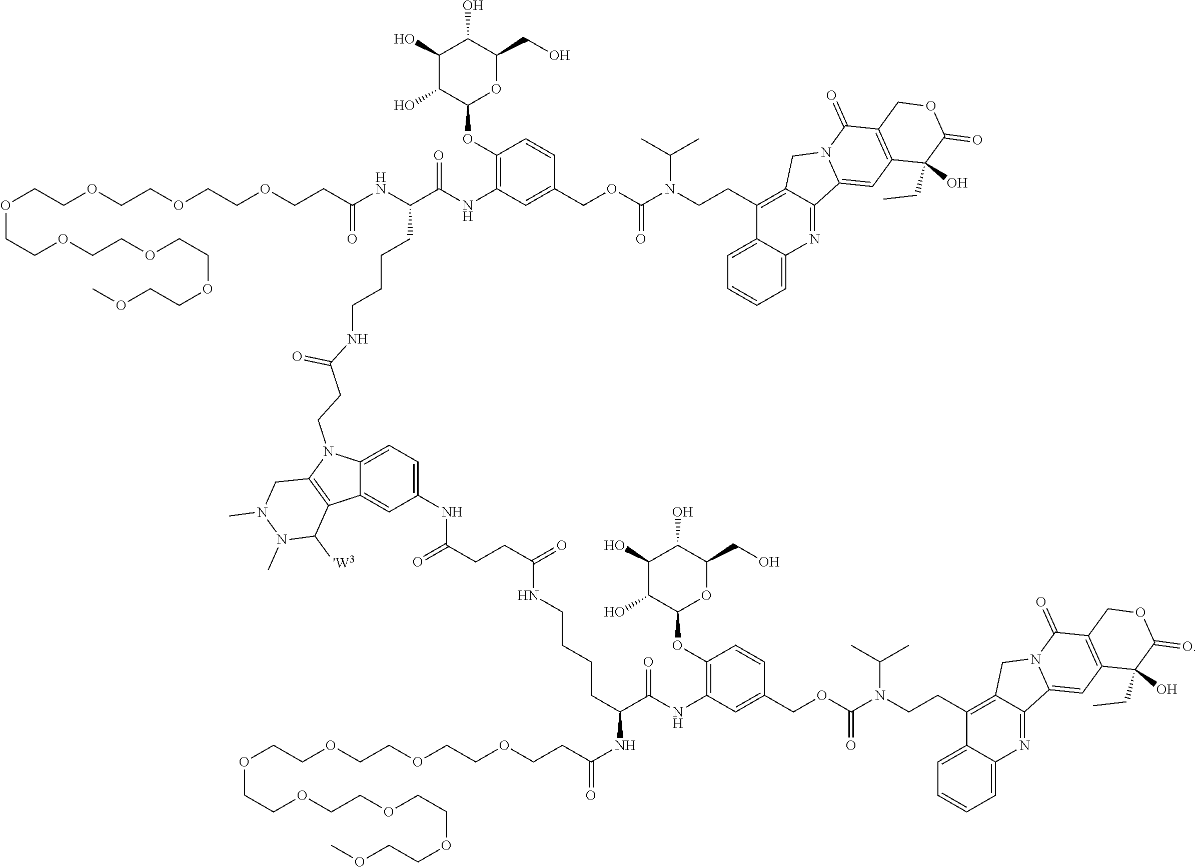

In some embodiments, the conjugate is selected from:

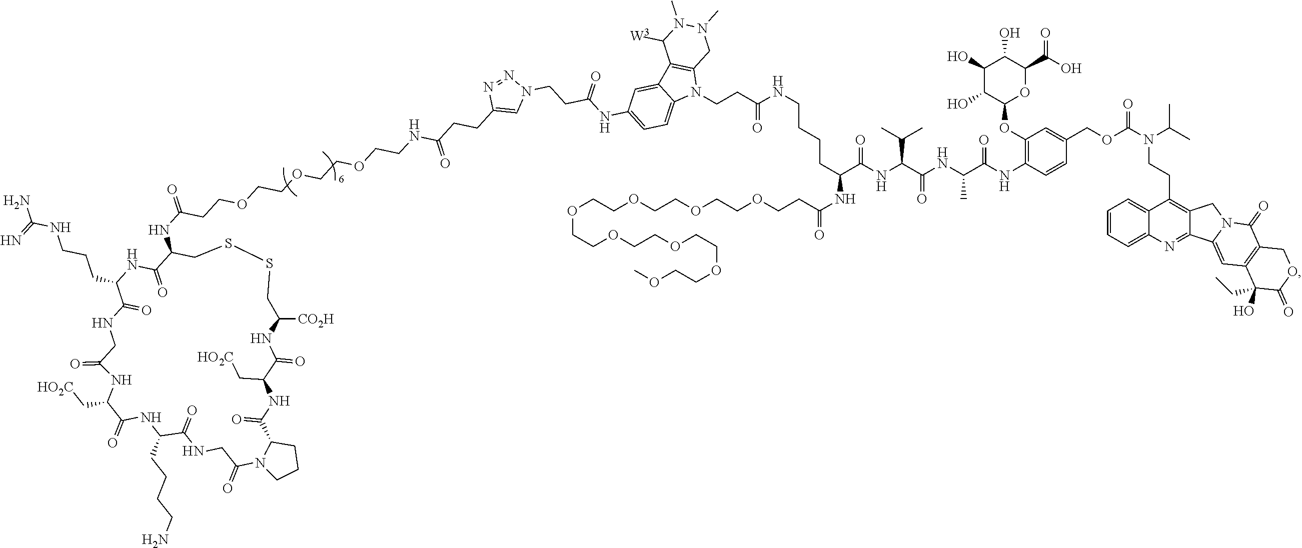

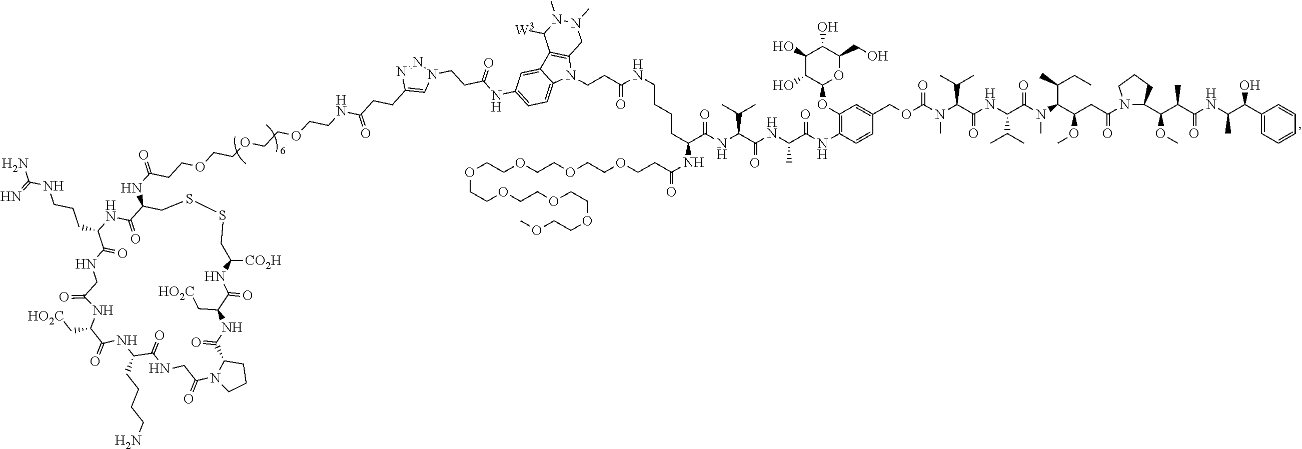

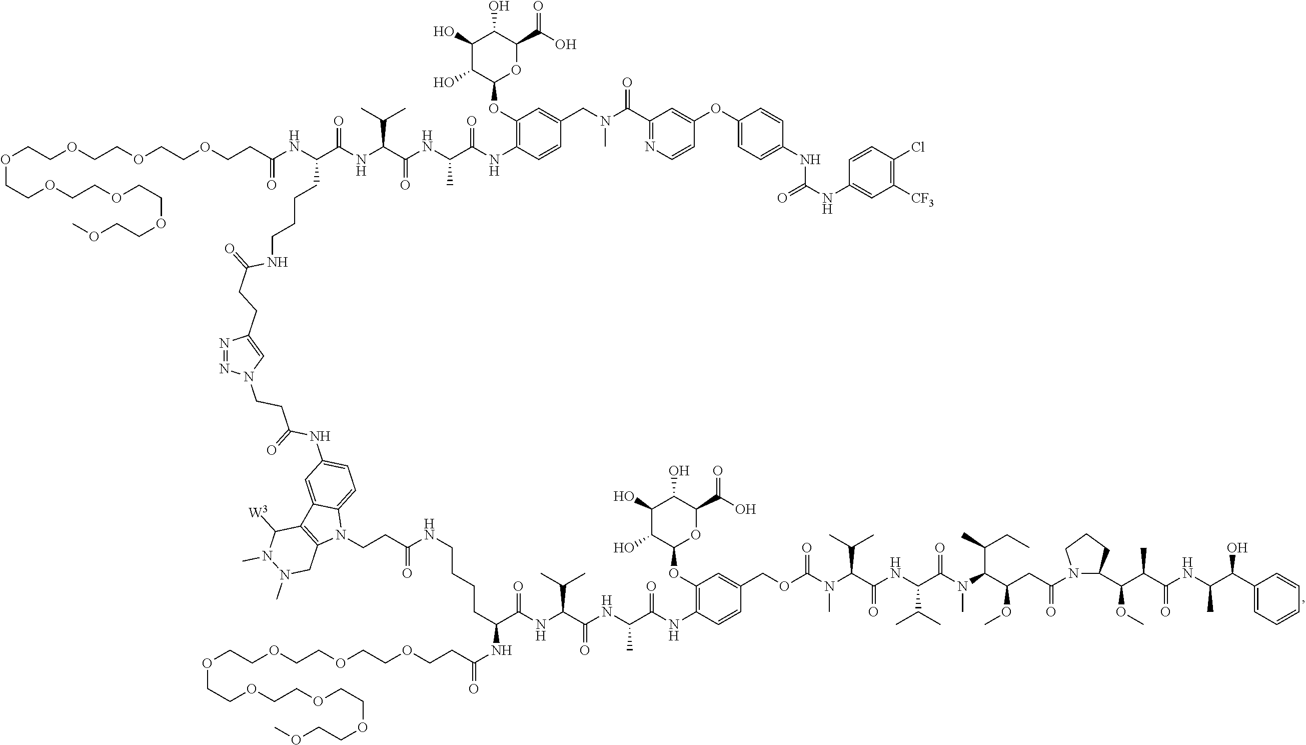

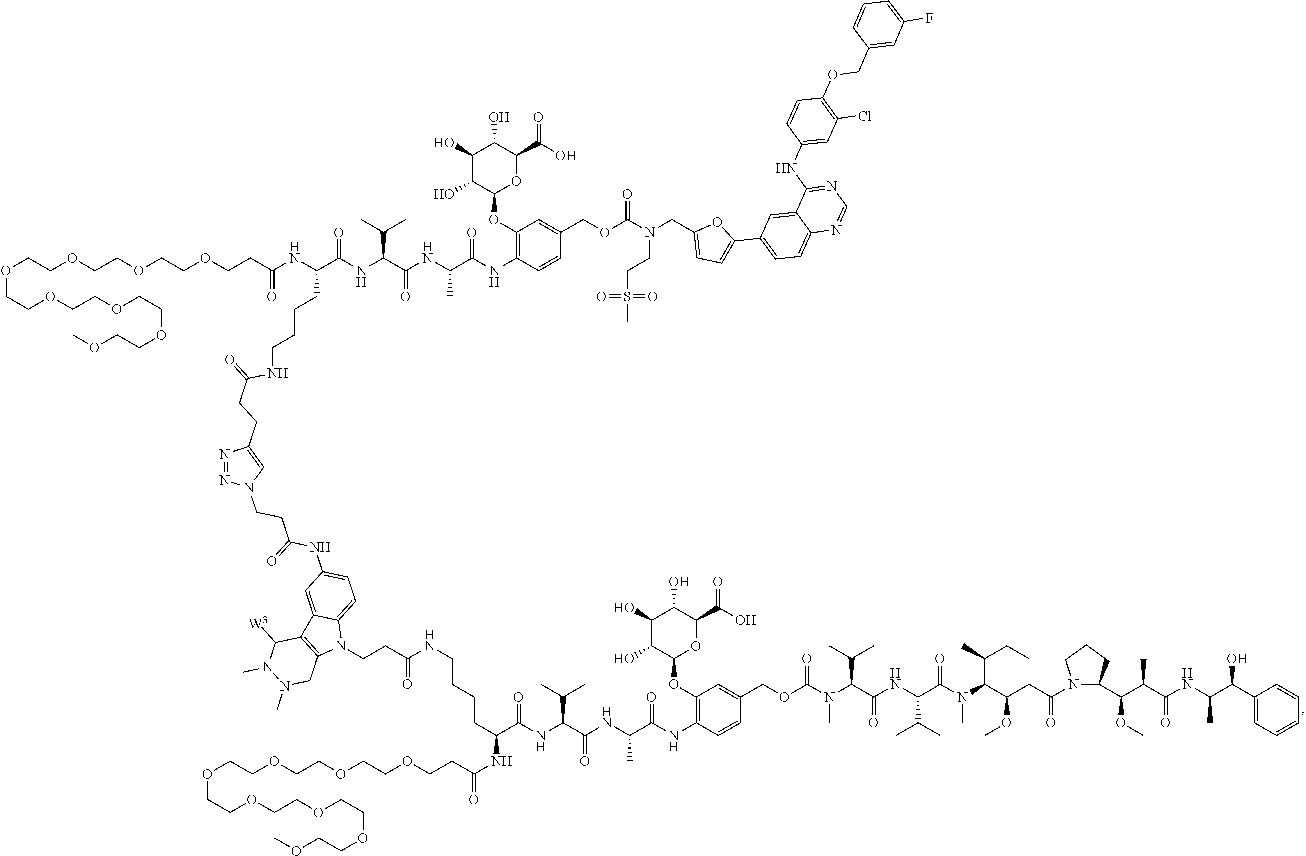

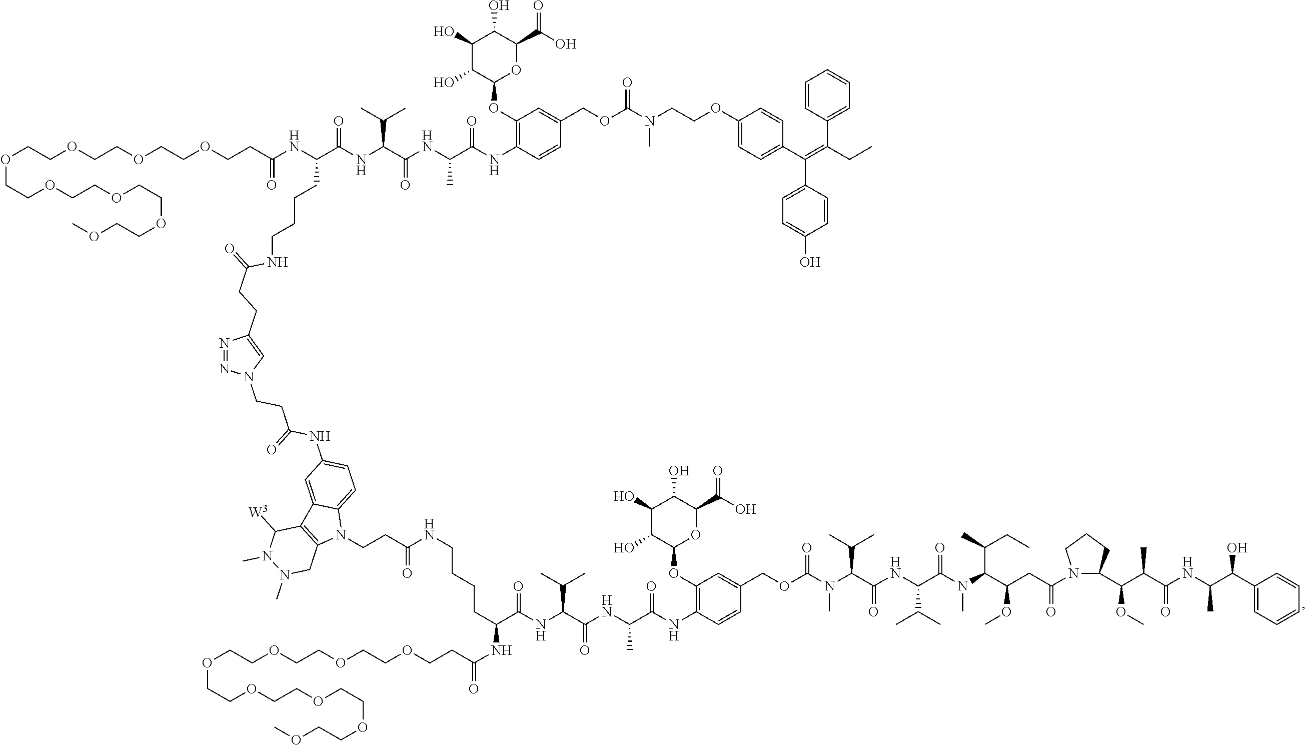

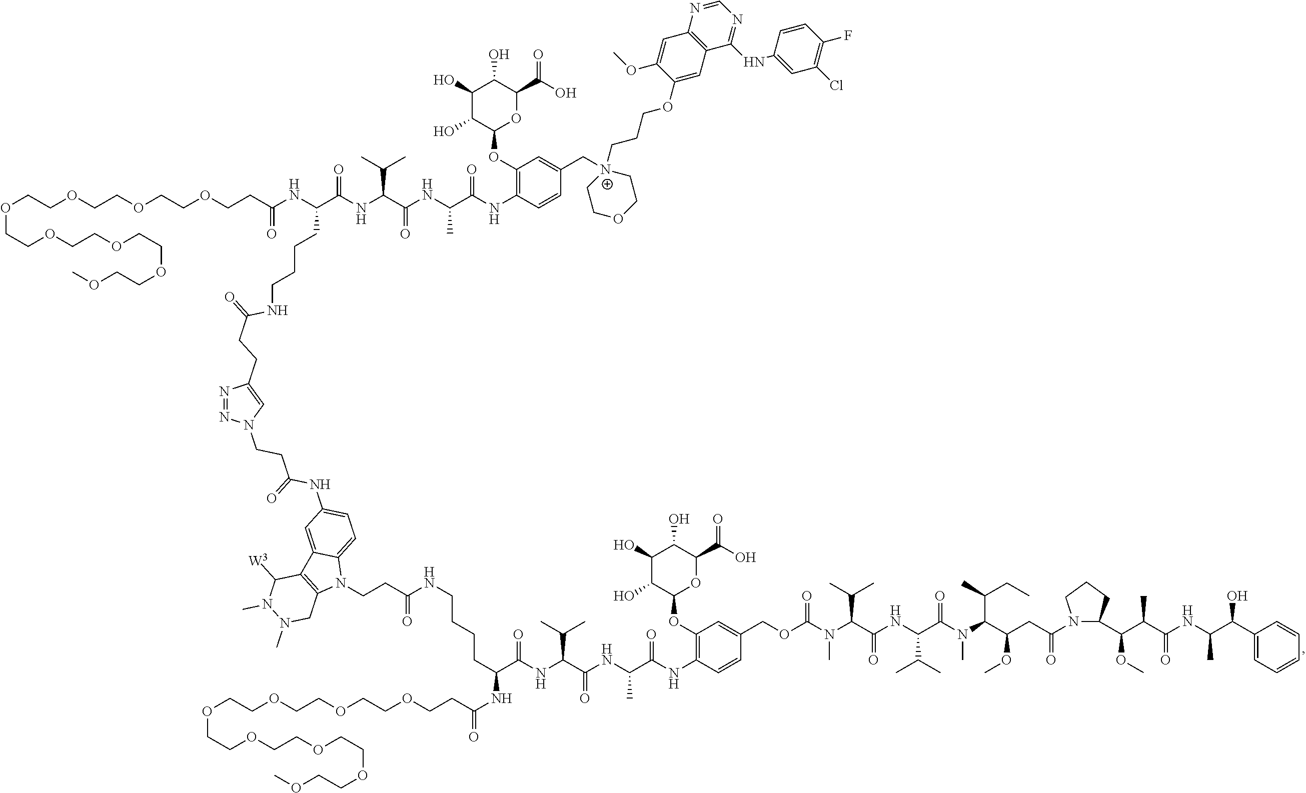

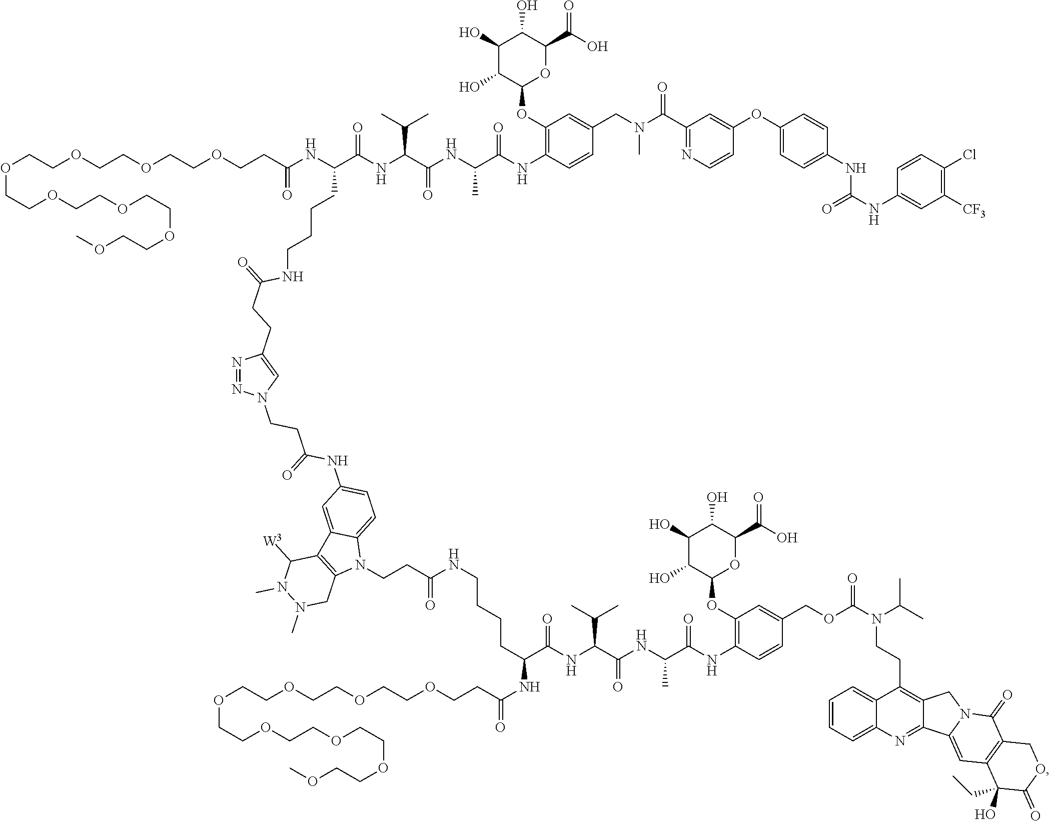

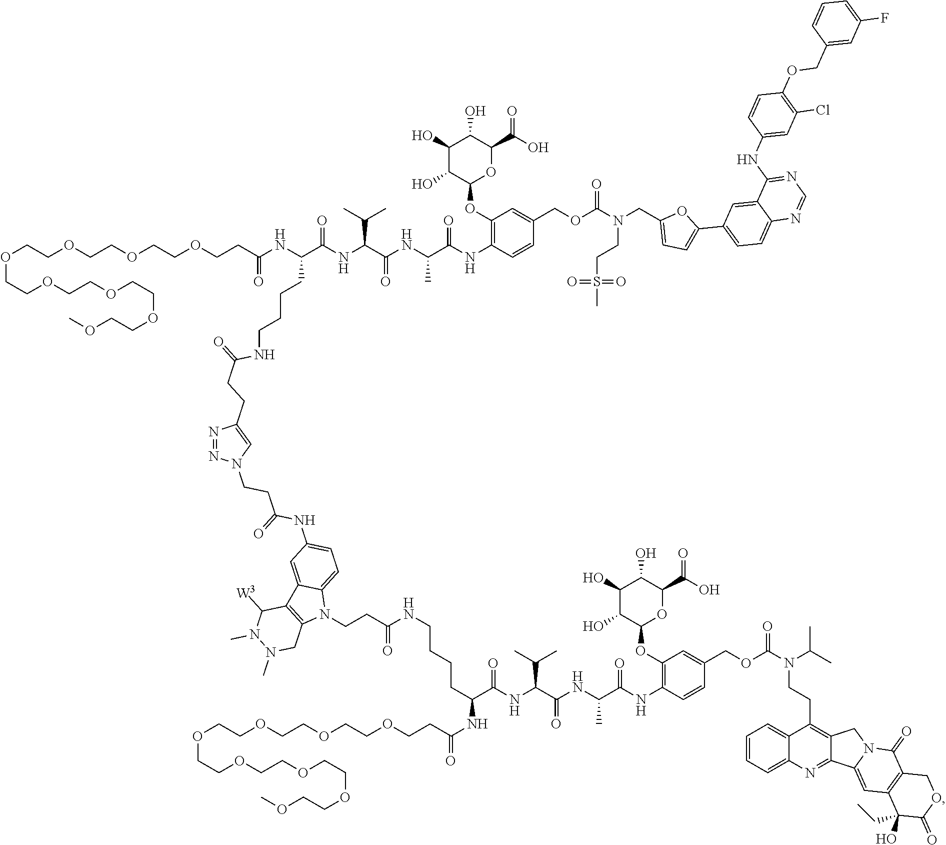

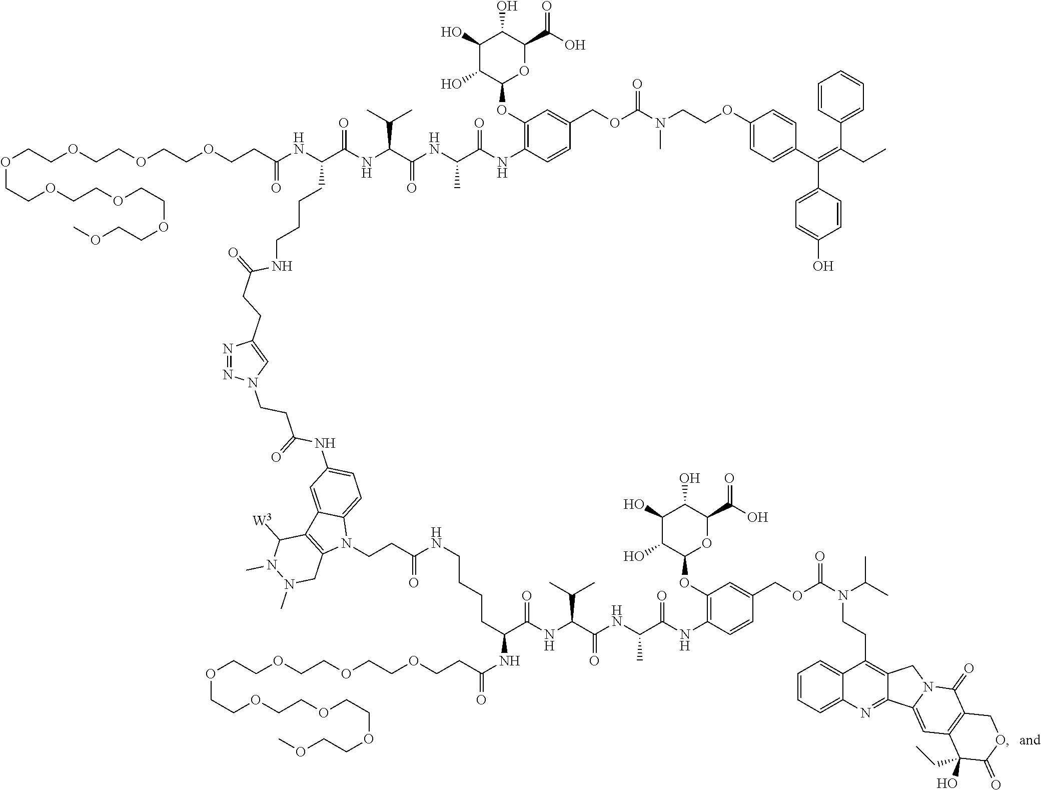

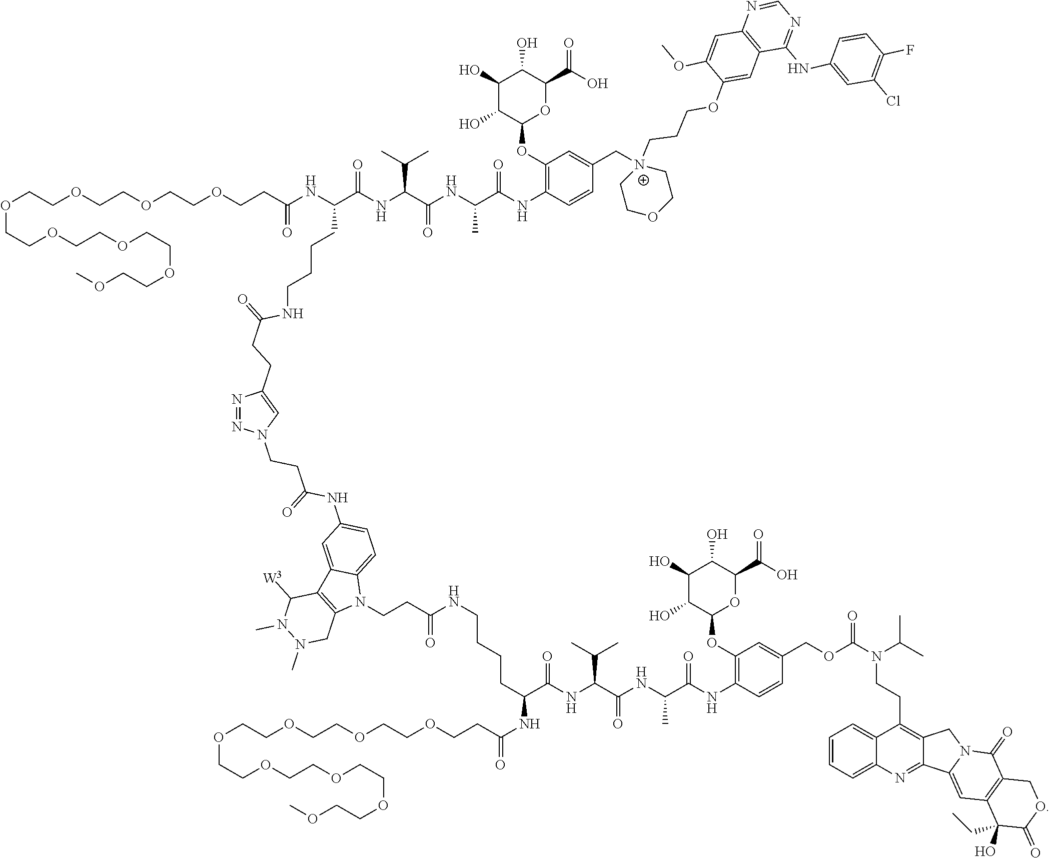

Aspects of the present disclosure include a compound of formula (II):

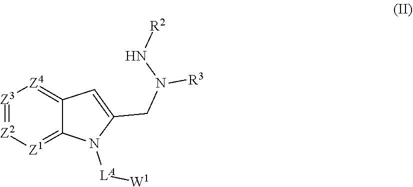

wherein:
   Z¹, Z², Z³ and Z⁴ are each independently selected from CR⁴, N and C-L^{B}-W², wherein at least one Z¹, Z², Z³ and Z⁴ is C-L^{B}-W²;
   R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
   each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
   L^{A} is a first linker;
   L^{B} is a second linker;
   W¹ is a first drug; and
   W² is a second drug.

In some embodiments, Z¹ is CR⁴.

In some embodiments, Z¹ is N.

In some embodiments, Z³ is C-L^{B}-W².

In some embodiments, L^{A} comprises:

-(T¹-V¹)ₐ-(T²-V²)_{b}-(T³-V³)_{c}-(T⁴-V⁴)_{d}-(T⁵-V⁵)ₑ-(T⁶-V⁶)_{f}—,
wherein
   a, b, c, d, e and f are each independently 0 or 1;
   T¹, T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
   V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NRCO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
   each R¹³ is independently selected from hydrogen, an alkyl, a substituted alkyl, an aryl, and a substituted aryl; and
   each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

In some embodiments, of L^{A}:
T¹ is selected from a (C₁-C₁₂)alkyl and a substituted (C₁-C₁₂)alkyl;
T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PHP, an acetal group, a hydrazine, and an ester; and
V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂—, and —P(O)OH—;
wherein:
(PEG)ₙ is

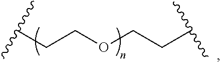

where n is an integer from 1 to 30;
EDA is an ethylene diamine moiety having the following structure:

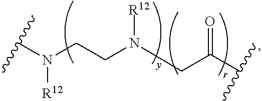

where y is an integer from 1 to 6 and r is 0 or 1;
4-amino-piperidine (4AP) is

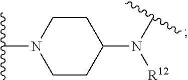

and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring.

In some embodiments, T¹, T², T³, T⁴, T⁵ and T⁶ are each optionally substituted with a glycoside.

In some embodiments, MABO, MABC, PABO, PABC, PAB, PABA, PAP and PHP are each optionally substituted with a glycoside.

In some embodiments, the glycoside is selected from a glucuronide, a galactoside, a glucoside, a mannoside, a fucoside, O-GlcNAc, and O-GalNAc.

In some embodiments, L^{A} is a linker wherein:
T¹ is (C₁-C₁₂)alkyl and V¹ is —CO—;
T² is AA and V² is absent;
T³ is PABC and V³ is absent; and
d, e and f are each 0; or
wherein:
T¹ is (C₁-C₁₂)alkyl and V¹ is —CONH—;
T² is (PEG)ₙ and V² is —CO—;
T³ is AA and V³ is absent;
T⁴ is PABC and V⁴ is absent; and
e and f are each 0; or
wherein:
T¹ is (C₁-C₁₂)alkyl and V¹ is —CO—;
T² is an amino acid analog and V² is —NH—;
T³ is (PEG)ₙ and V³ is —CO—;
T⁴ is AA and V⁴ is absent;
T⁵ is PABC and V⁵ is absent; and
f is 0; or
wherein:
T¹ is (C₁-C₁₂)alkyl and V¹ is —CONH—;
T² is (PEG)ₙ and V² is —CO—;
T³ is AA and V³ is absent;
T⁴ is PABC and V⁴ is absent; and
e and f are each 0; or
wherein:
T¹ is (C₁-C₁₂)alkyl and V¹ is —CONH—;
T² is substituted (C₁-C₁₂)alkyl and V² is —CO—;
T³ is AA and V³ is absent;
T⁴ is PABC and V⁴ is absent; and
e and f are each 0; or
wherein:
T¹ is (C₁-C₁₂)alkyl and V¹ is —CONH—;
T² is (PEG)ₙ and V² is —CO—;
T³ is AA and V³ is absent;
T⁴ is PABA and V⁴ is —CO—;
T⁵ is (C₁-C₁₂)alkyl and V⁵ is absent; and
f is 0; or
wherein:
T¹ is (C₁-C₁₂)alkyl and V¹ is —CO—;
T² is 4AP and V² is —CO—;
T³ is (C₁-C₁₂)alkyl and V³ is —CO—;
T⁴ is AA and V⁴ is absent;
T⁵ is PABC and V⁵ is absent; and
f is 0; or
wherein:
T¹ is (C₁-C₁₂)alkyl and V¹ is —CO—;
T² is 4AP and V² is —CO—;
T³ is (C₁-C₁₂)alkyl and V³ is —O—;
T⁴ is (C₁-C₁₂)alkyl and V⁴ is —CO—;
T⁵ is AA and V⁵ is absent; and
T⁶ is PABC and V⁶ is absent; or
wherein:
T¹ is (C₁-C₁₂)alkyl and V¹ is —CO—;
T² is an amino acid analog and V² is absent;
T³ is AA and V³ is absent;
T⁴ is PABC and V⁴ is absent; and
e and f are each 0; or
wherein:
T¹ is (C₁-C₁₂)alkyl and V¹ is —CONH—;
T² is (PEG)ₙ and V² is —CONH—;
T³ is substituted (C₁-C₁₂)alkyl and V³ is —CO—;
T⁴ is AA and V⁴ is absent;
T⁵ is PABC and V⁵ is absent; and
f is 0; or
wherein:
T¹ is (C₁-C₁₂)alkyl and V¹ is —CO—;
T² is an AA and V² is —NH—;
T³ is (PEG)ₙ and V³ is —CO—;
T⁴ is AA and V⁴ is absent;
T⁵ is PABC and V⁵ is absent; and
f is 0; or
wherein:
T¹ is (C₁-C₁₂)alkyl and V¹ is —CONH—;
T² is (PEG)ₙ and V² is —CO—;
T³ is AA and V³ is absent;
T⁴ is PAP and V⁴ is —C(O)O—; and
e and f are each 0; or
wherein:
T¹ is (C₁-C₁₂)alkyl and V¹ is —CONH—;
T² is substituted (C₁-C₁₂)alkyl and V² is —CO—;
T³ is AA and V³ is absent;
T⁴ is PABC and V⁴ is absent; and
e and f are each 0; or
wherein:
T¹ is (C₁-C₁₂)alkyl and V¹ is —CONH—;
T² is substituted (C₁-C₁₂)alkyl and V² is —CO—;
T³ is PABC and V³ is absent; and
d, e and f are each 0.

In some embodiments, L^{B} comprises:

-(T⁷-V⁷)_{g}-(T⁸-V⁸)ₕ-(T⁹-V⁹)ᵢ-(T¹⁰-V¹⁰)ⱼ-(T¹¹V¹¹)ₖ-(T¹²-V¹²)ₗ-(T¹³V¹³)ₘ,
wherein
   g, h, i, j, k and 1 are each independently 0 or 1;
   T⁷, T⁸, T⁹, T¹⁰, T¹¹, T¹² and T¹³ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
   V⁷, V⁸, V⁹, V¹⁰, V¹¹, V¹² and V¹³ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
   each R¹³ is independently selected from hydrogen, an alkyl, a substituted alkyl, an aryl, and a substituted aryl; and
   each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

In some embodiments, T⁷, T⁸, T⁹, T¹⁰, T¹¹. T¹² and T¹³ are each optionally substituted with a glycoside.

In some embodiments, MABO, MABC, PABO, PABC, PAB, PABA, PAP and PHP are each optionally substituted with a glycoside.

In some embodiments, the glycoside is selected from a glucuronide, a galactoside, a glucoside, a mannoside, a fucoside, O-GlcNAc, and O-GalNAc.

In some embodiments, L^{B} is a linker wherein:
T⁷ is absent and V⁷ is —NHCO—;
T⁸ is (C₁-C₁₂)alkyl and V⁸ is —CO—;
T⁹ is AA and V⁹ is absent;
T¹⁰ is PABC and V¹⁰ is absent; and
k, l and m are each 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is AA and V¹⁰ is absent; and
T¹¹ is PABC and V¹¹ is absent; and
l and m are each 0; or
wherein:
T⁷ is absent and V⁷ is —NHCO—;
T⁸ is (C₁-C₁₂)alkyl and V⁸ is —CO—;
T⁹ is an amino acid analog and V⁹ is —NH—;
T¹⁰ is (PEG)ₙ and V¹⁰ is —CO—;
T¹¹ is AA and V¹¹ is absent;
T¹² is PABC and V¹² is absent; and
m is 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is AA and V¹⁰ is absent;
T¹¹ is PABC and V¹¹ is absent; and
l and m are each 0; or
wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is AA and V¹⁰ is absent;
T¹¹ is PABC and V¹¹ is absent; and
l and m are each 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is AA and V¹⁰ is absent;
T¹¹ is PABA and V¹¹ is —CO—;
T¹² is (C₁-C₁₂)alkyl and V¹² is absent; and
m is 0; or
wherein:
T⁷ is absent and V⁷ is —NHCO—;
T⁸ is (C₁-C₁₂)alkyl and V⁸ is —CO—;
T⁹ is 4AP and V⁹ is —CO—;
T¹⁰ is (C₁-C₁₂)alkyl and V¹⁰ is —CO—;
T¹¹ is AA and V¹¹ is absent;
T¹² is PABC and V¹² is absent; and
m is 0; or
wherein:
T⁷ is absent and V⁷ is —NHCO—;
T⁸ is (C₁-C₁₂)alkyl and V⁸ is —CO—;
T⁹ is 4AP and V⁹ is —CO—;
T¹⁰ is (C₁-C₁₂)alkyl and V¹⁰ is —O—;
T¹¹ is (C₁-C₁₂)alkyl and V¹¹ is —CO—;
T¹² is AA and V¹² is absent; and
T¹³ PABC and V¹³ is absent; or
wherein:
T⁷ is absent and V⁷ is —NHCO—;
T⁸ is (C₁-C₁₂)alkyl and V⁸ is —CO—;
T⁹ is an amino acid analog and V⁹ is absent;
T¹⁰ is AA and V¹⁰ is absent;
T¹¹ is PABC and V¹¹ is absent; and
l and m are each 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CONH—;
T¹⁰ is substituted (C₁-C₁₂)alkyl and V¹⁰ is —CO—;
T¹¹ is AA and V¹¹ is absent;
T¹² is PABC and V¹² is absent; and
m is 0; or
wherein:
T⁷ is absent and V⁷ is —NHCO—;
T⁸ is (C₁-C₁₂)alkyl and V⁸ is —CO—;
T⁹ is AA and V⁹ is —NH—;
T¹⁰ is (PEG)ₙ and V¹⁰ is —CO—;
T¹¹ is AA and V¹¹ is absent;
T¹² is PABC and V¹² is absent; and
m is 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is AA and V¹⁰ is absent;
T¹¹ is PAP and V¹¹ is —C(O)O—; and
l and m are each 0; or
wherein:
T⁷ is absent and V⁷ is —NHCO—;
T⁸ is (C₁-C₁₂)alkyl and V⁸ is —CO—;
T⁹ is AA and V⁹ is absent;
T¹⁰ is PABC and V¹⁰ is absent;
T¹¹ is PAP and V¹¹ is —C(O)O—; and
l and m are each 0; or
wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is PABC and V¹⁰ is absent; and
k, l and m are each 0; or
wherein:
T⁷ is absent and V⁷ is —NHCO—;
T⁸ is (C₁-C₁₂)alkyl and V⁸ is absent;
T⁹ is heteroaryl and V⁹ is absent;
T¹⁰ is (C₁-C₁₂)alkyl and V¹⁰ is —CONH—;
T¹¹ is (PEG)ₙ and V¹¹ is —CO—; and
l and m are each 0; or
wherein:
T⁷ is absent and V⁷ is —NHCO—;
T⁸ is (C₁-C₁₂)alkyl and V⁸ is absent;
T⁹ is heteroaryl and V⁹ is absent;
T¹⁰ is (C₁-C₁₂)alkyl and V¹⁰ is —CONH—;
T¹¹ is substituted (C₁-C₁₂)alkyl and V¹¹ is —CO—;
T¹² is AA and V¹² is absent; and
T¹³ PAB and V¹³ is absent; or
wherein:
T⁷ is absent and V⁷ is —NHCO—;
T⁸ is (C₁-C₁₂)alkyl and V⁸ is absent;
T⁹ is heteroaryl and V⁹ is absent;
T¹⁰ is (C₁-C₁₂)alkyl and V¹⁰ is —CONH—;
T¹¹ is substituted (C₁-C₁₂)alkyl and V¹¹ is —CO—;
T¹² is AA and V¹² is absent; and
T¹³ PABC and V¹³ is absent.

In some embodiments, the compound is selected from:

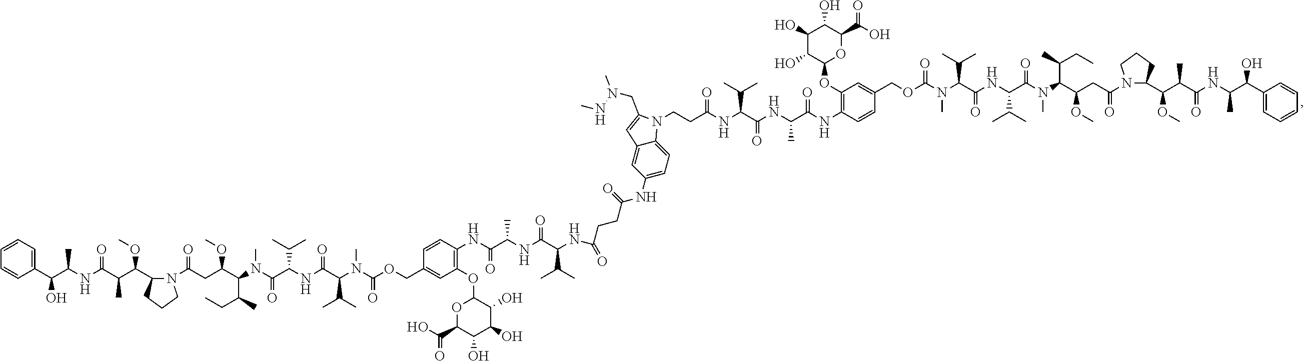

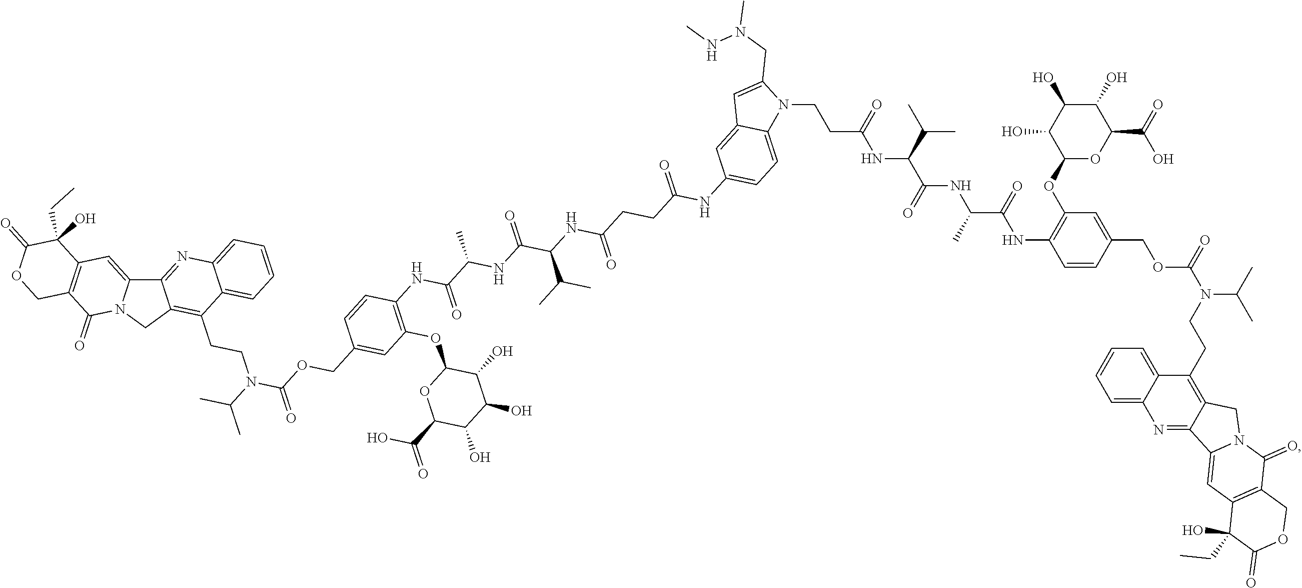 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCC(=O)Nc2ccc3c(c2)cc(CN(C)NC)n3CCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)c(O[C@@H]2O[C@H](C(=O)O)[C@@H](O)[C@H](O)[C@H]2O)c1)C(C)C]

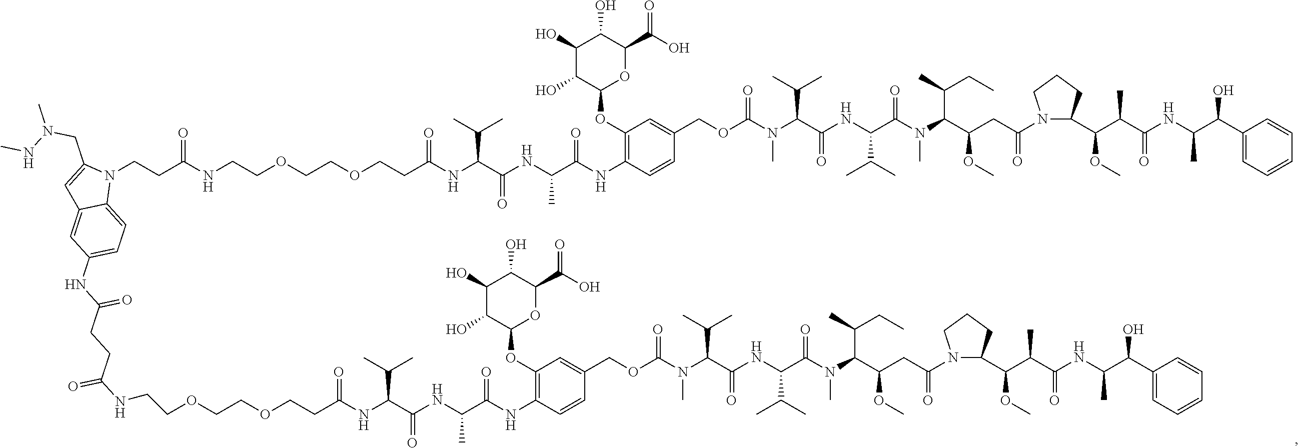

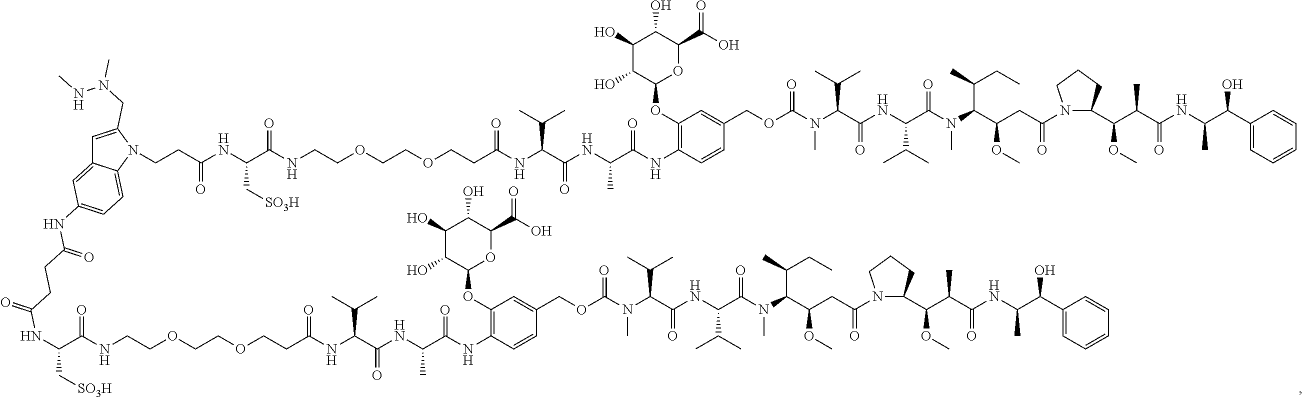

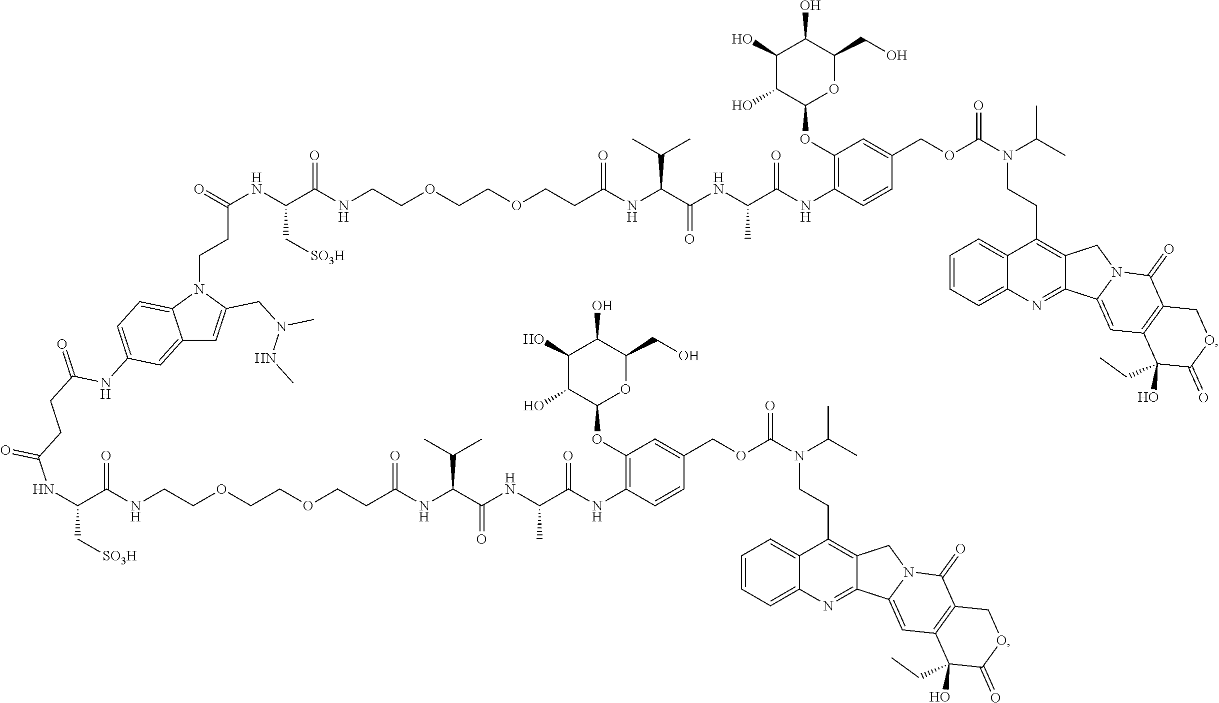 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCOCCOCCNC(=O)[C@H](CS(=O)(=O)O)NC(=O)CCC(=O)Nc2ccc3c(c2)cc(CN(C)NC)n3CCC(=O)N[C@@H](CS(=O)(=O)O)C(=O)NCCOCCOCCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](CO)[C@H](O)[C@H](O)[C@H]2O)C(C)C)C(C)C)c(O[C@@H]2O[C@H](CO)[C@H](O)[C@H](O)[C@H]2O)c1)C(C)C]

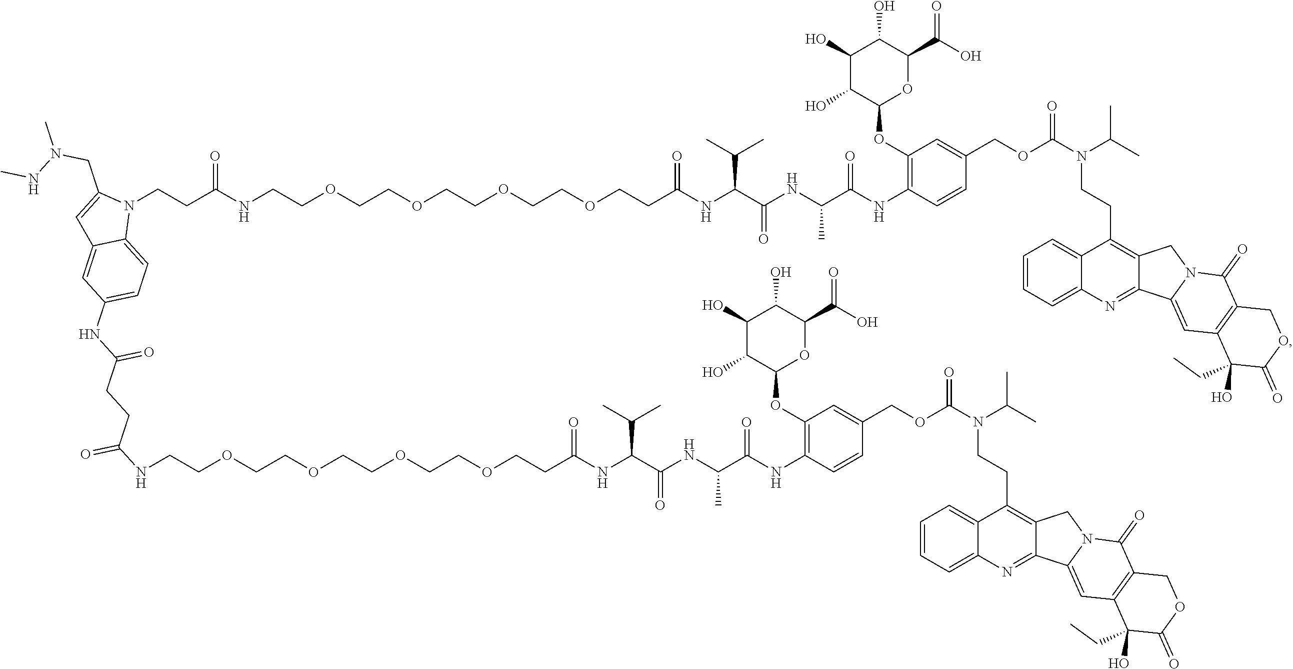 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCOCCOCCOCCOCCNC(=O)CCC(=O)Nc2ccc3c(c2)cc(CN(C)NC)n3CCC(=O)NCCOCCOCCOCCOCCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)c(O[C@@H]2O[C@H](C(=O)O)[C@@H](O)[C@H](O)[C@H]2O)c1)C(C)C]

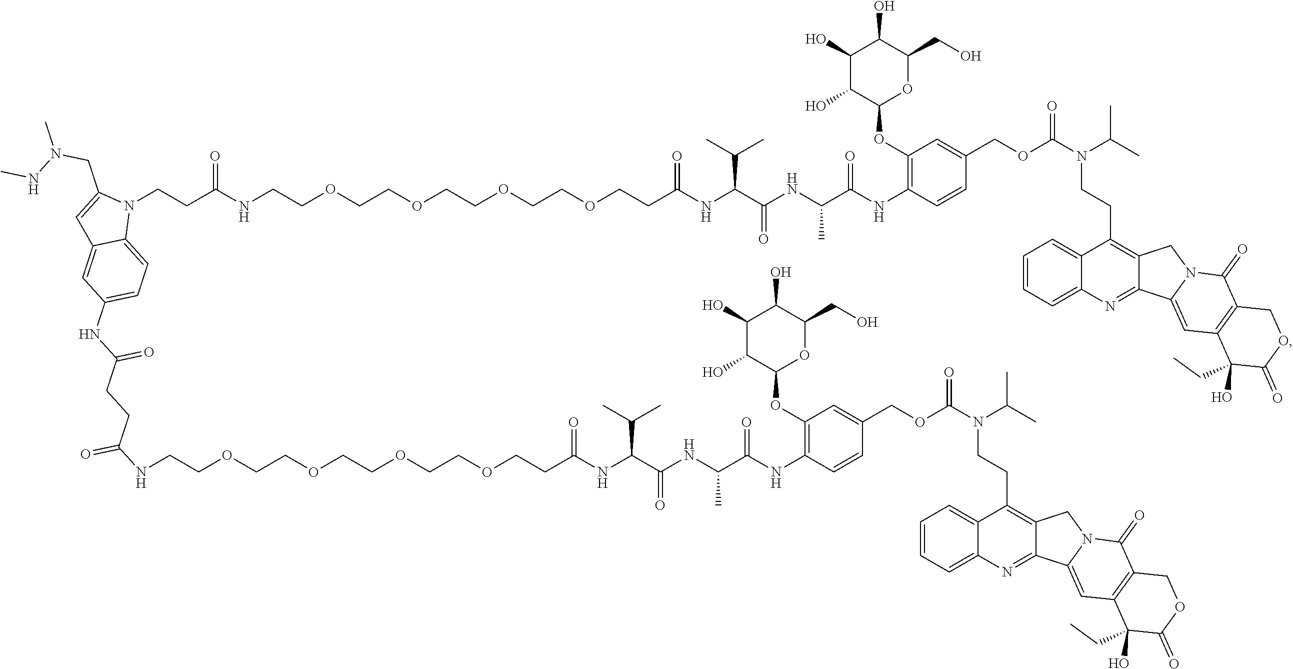 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCOCCOCCOCCOCCNC(=O)CCC(=O)Nc2ccc3c(c2)cc(CN(C)NC)n3CCC(=O)NCCOCCOCCOCCOCCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](CO)[C@H](O)[C@H](O)[C@H]2O)C(C)C)C(C)C)c(O[C@@H]2O[C@H](CO)[C@H](O)[C@H](O)[C@H]2O)c1)C(C)C]

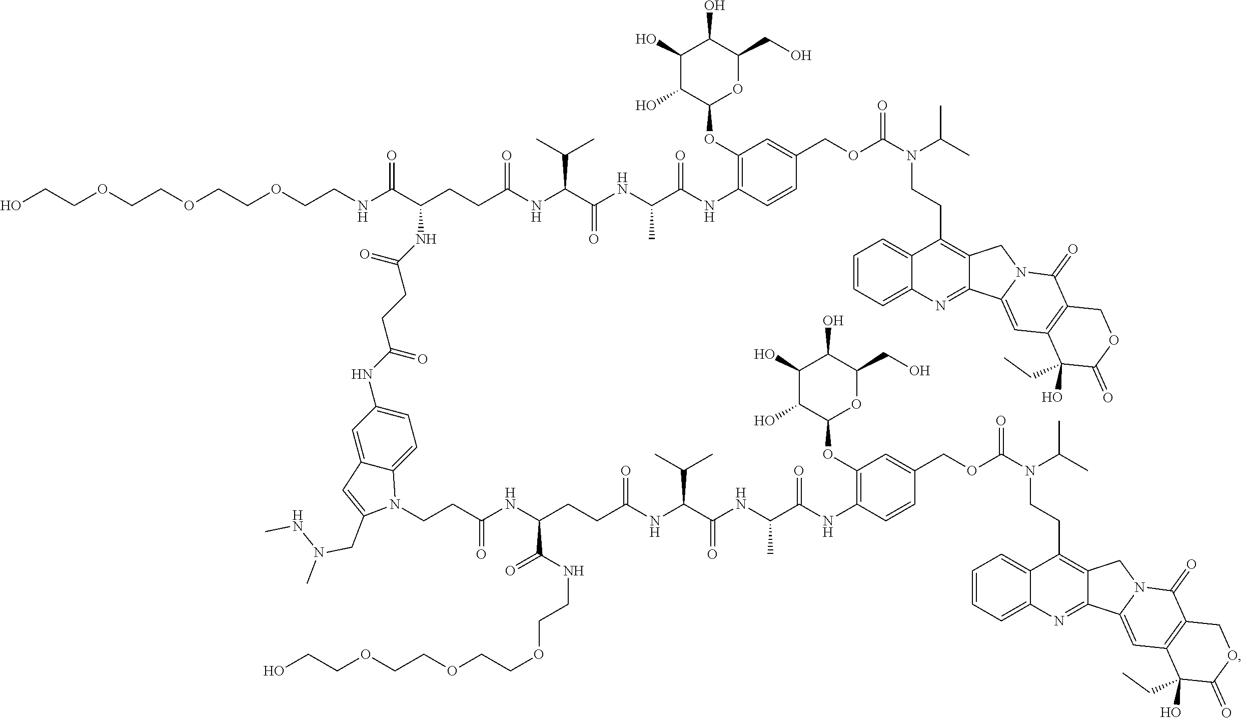 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CC[C@H](NC(=O)CCC(=O)Nc2ccc3c(c2)cc(CN(C)NC)n3CCC(=O)N[C@@H](CCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](CO)[C@H](O)[C@H](O)[C@H]2O)C(C)C)C(=O)NCCOCCOCCOCCO)C(=O)NCCOCCOCCOCCO)C(C)C)c(O[C@@H]2O[C@H](CO)[C@H](O)[C@H](O)[C@H]2O)c1)C(C)C]

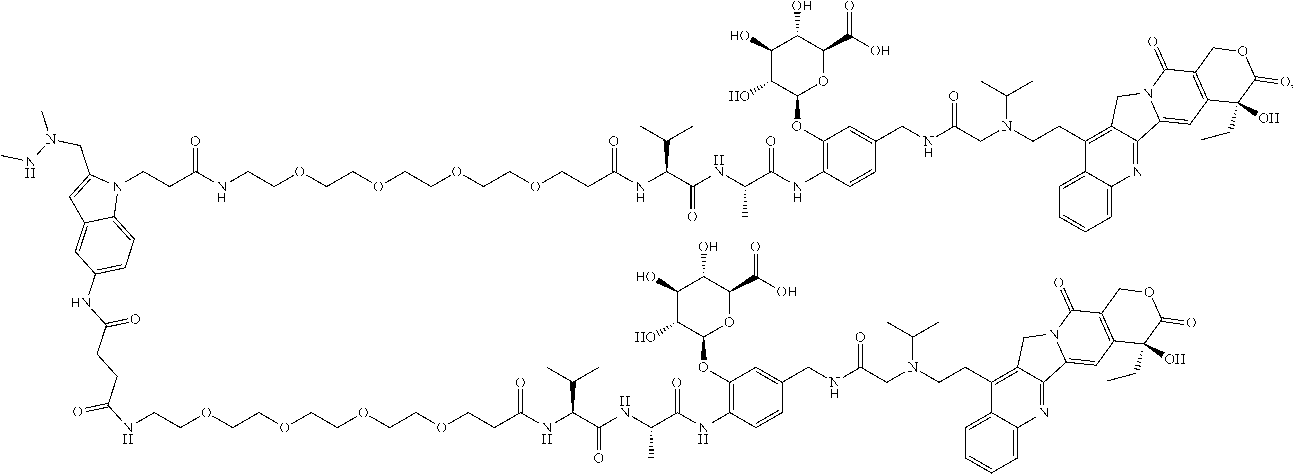 [SMILES: CC[C@@]1(O)C(=O)OCc2c1cc1n(c2=O)Cc2c-1nc1ccccc1c2CCN(CC(=O)NCc1ccc(NC(=O)[C@H](C)NC(=O)[C@@H](NC(=O)CCOCCOCCOCCOCCNC(=O)CCC(=O)Nc2ccc3c(c2)cc(CN(C)NC)n3CCC(=O)NCCOCCOCCOCCOCCC(=O)N[C@H](C(=O)N[C@@H](C)C(=O)Nc2ccc(CNC(=O)CN(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)c(O[C@@H]2O[C@H](C(=O)O)[C@@H](O)[C@H](O)[C@H]2O)c1)C(C)C]

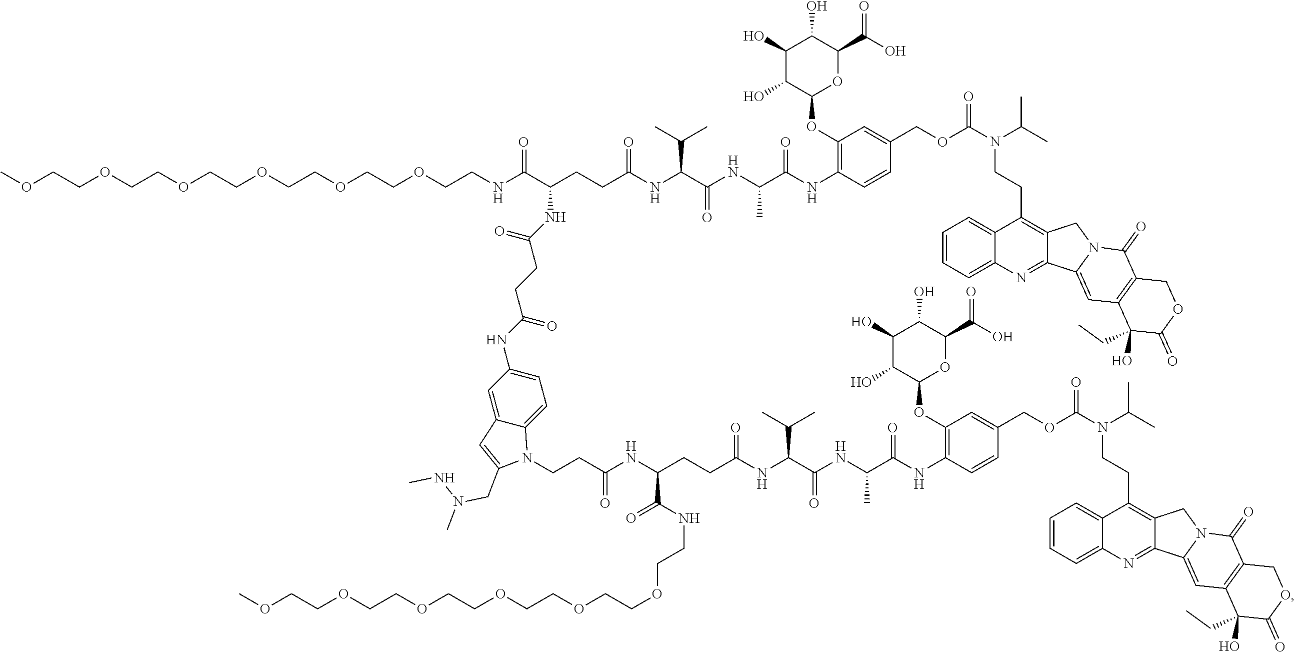 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CC[C@H](NC(=O)CCC(=O)Nc2ccc3c(c2)cc(CN(C)NC)n3CCC(=O)N[C@@H](CCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(=O)NCCOCCOCCOCCOCCOCCOC)C(=O)NCCOCCOCCOCCOCCOCCOC)C(C)C)c(O[C@@H]2O[C@H](C(=O)O)[C@@H](O)[C@H](O)[C@H]2O)c1)C(C)C]

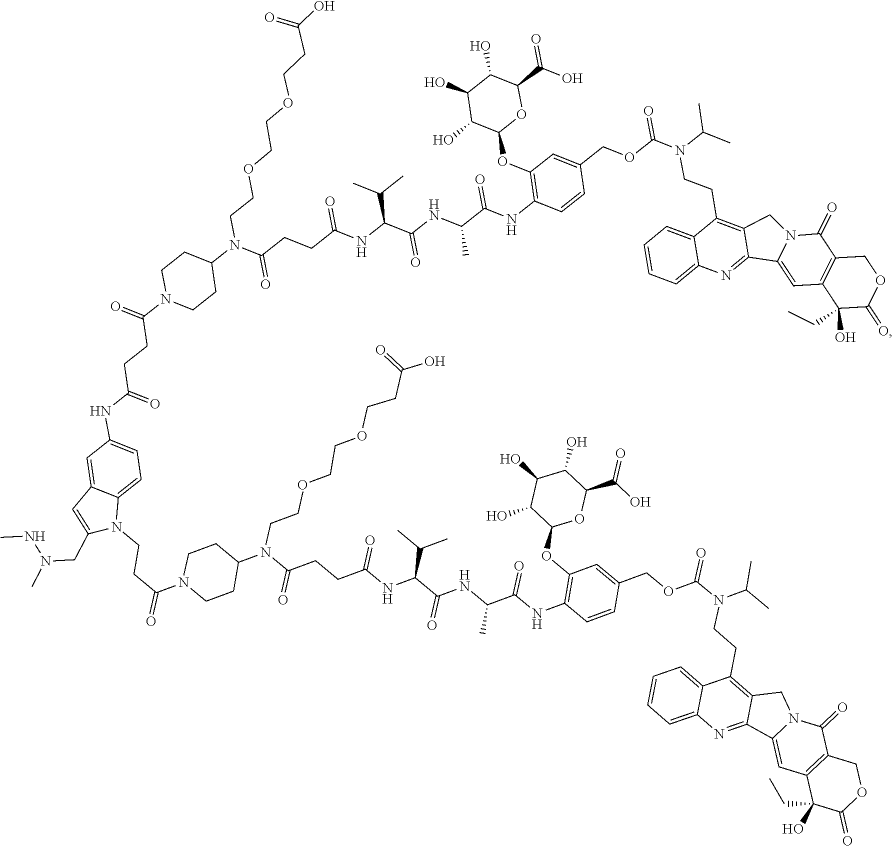 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCC(=O)N(CCOCCOCCC(=O)O)C2CCN(C(=O)CCC(=O)Nc3ccc4c(c3)cc(CN(C)NC)n4CCC(=O)N3CCC(N(CCOCCOCCC(=O)O)C(=O)CCC(=O)N[C@H](C(=O)N[C@@H](C)C(=O)Nc4ccc(COC(=O)N(CCc5c6c(nc7ccccc57)-c5cc7c(c(=O)n5C6)COC(=O)[C@]7(O)CC)C(C)C)cc4O[C@@H]4O[C@H](C(=O)O)[C@@H](O)[C@H](O)[C@H]4O)C(C)C)CC3)CC2)C(C)C)c(O[C@@H]2O[C@H](C(=O)O)[C@@H](O)[C@H](O)[C@H]2O)c1)C(C)C]

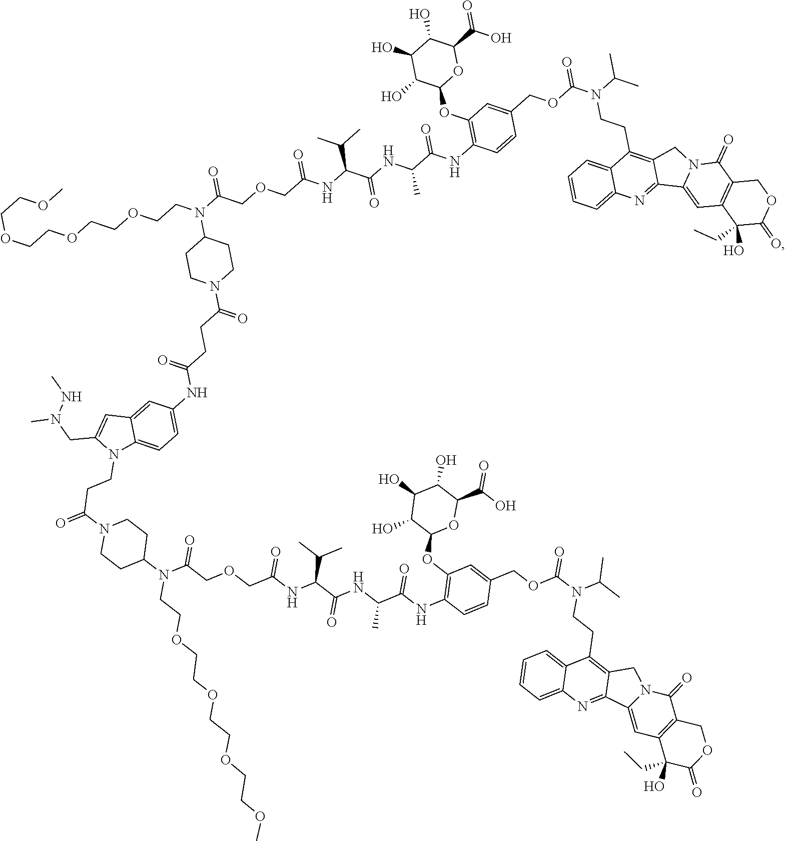 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)COCC(=O)N(CCOCCOCCOCCOC)C2CCN(C(=O)CCC(=O)Nc3ccc4c(c3)cc(CN(C)NC)n4CCC(=O)N3CCC(N(CCOCCOCCOCCOC)C(=O)COCC(=O)N[C@H](C(=O)N[C@@H](C)C(=O)Nc4ccc(COC(=O)N(CCc5c6c(nc7ccccc57)-c5cc7c(c(=O)n5C6)COC(=O)[C@]7(O)CC)C(C)C)cc4O[C@@H]4O[C@H](C(=O)O)[C@@H](O)[C@H](O)[C@H]4O)C(C)C)CC3)CC2)C(C)C)c(O[C@@H]2O[C@H](C(=O)O)[C@@H](O)[C@H](O)[C@H]2O)c1)C(C)C]

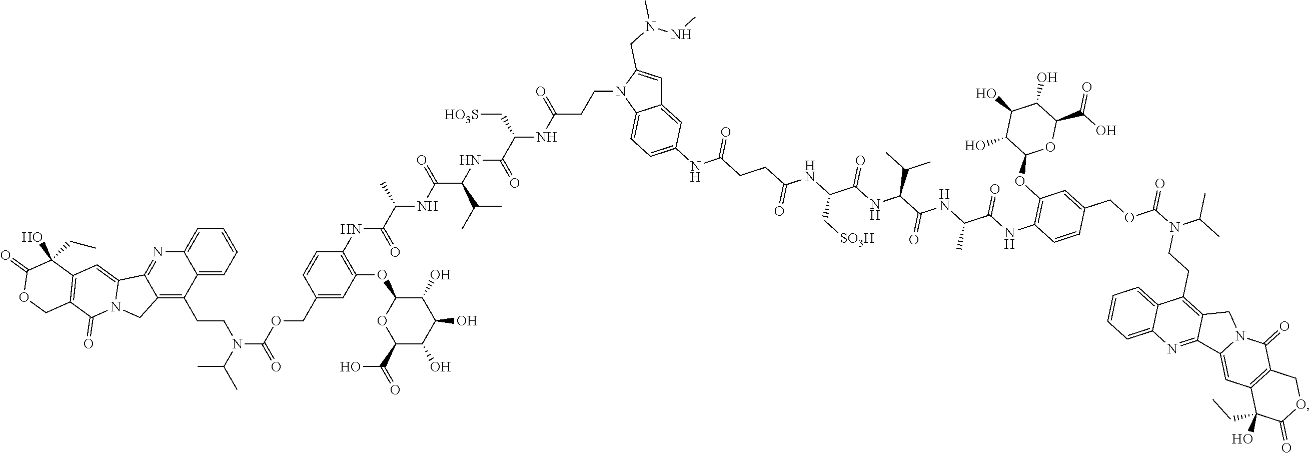 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CS(=O)(=O)O)NC(=O)CCC(=O)Nc2ccc3c(c2)cc(CN(C)NC)n3CCC(=O)N[C@@H](CS(=O)(=O)O)C(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)c(O[C@@H]2O[C@H](C(=O)O)[C@@H](O)[C@H](O)[C@H]2O)c1)C(C)C]

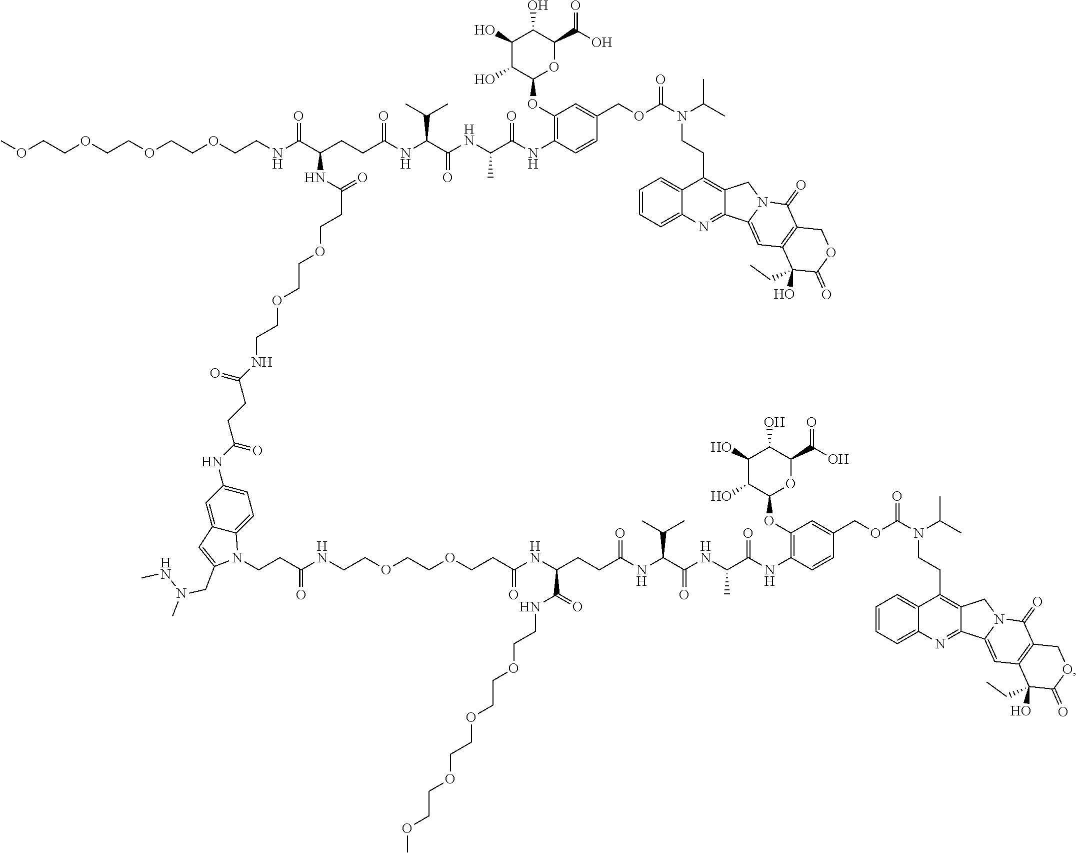 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CC[C@H](NC(=O)CCOCCOCCNC(=O)CCn2c(CN(C)NC)cc3cc(NC(=O)CCC(=O)NCCOCCOCCC(=O)N[C@H](CCC(=O)N[C@H](C(=O)N[C@@H](C)C(=O)Nc4ccc(COC(=O)N(CCc5c6c(nc7ccccc57)-c5cc7c(c(=O)n5C6)COC(=O)[C@]7(O)CC)C(C)C)cc4O[C@@H]4O[C@H](C(=O)O)[C@@H](O)[C@H](O)[C@H]4O)C(C)C)C(=O)NCCOCCOCCOCCOC)ccc32)C(=O)NCCOCCOCCOCCOC)C(C)C)c(O[C@@H]2O[C@H](C(=O)O)[C@@H](O)[C@H](O)[C@H]2O)c1)C(C)C]

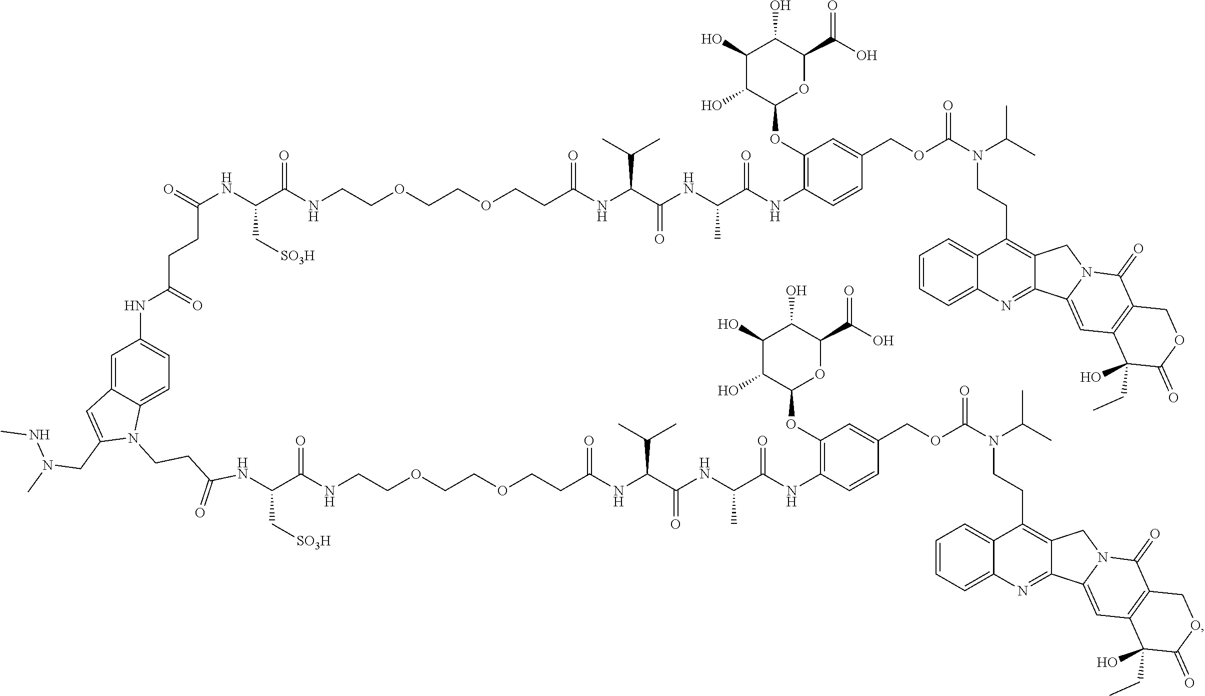 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCOCCOCCNC(=O)[C@H](CS(=O)(=O)O)NC(=O)CCC(=O)Nc2ccc3c(c2)cc(CN(C)NC)n3CCC(=O)N[C@@H](CS(=O)(=O)O)C(=O)NCCOCCOCCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)c(O[C@@H]2O[C@H](C(=O)O)[C@@H](O)[C@H](O)[C@H]2O)c1)C(C)C]

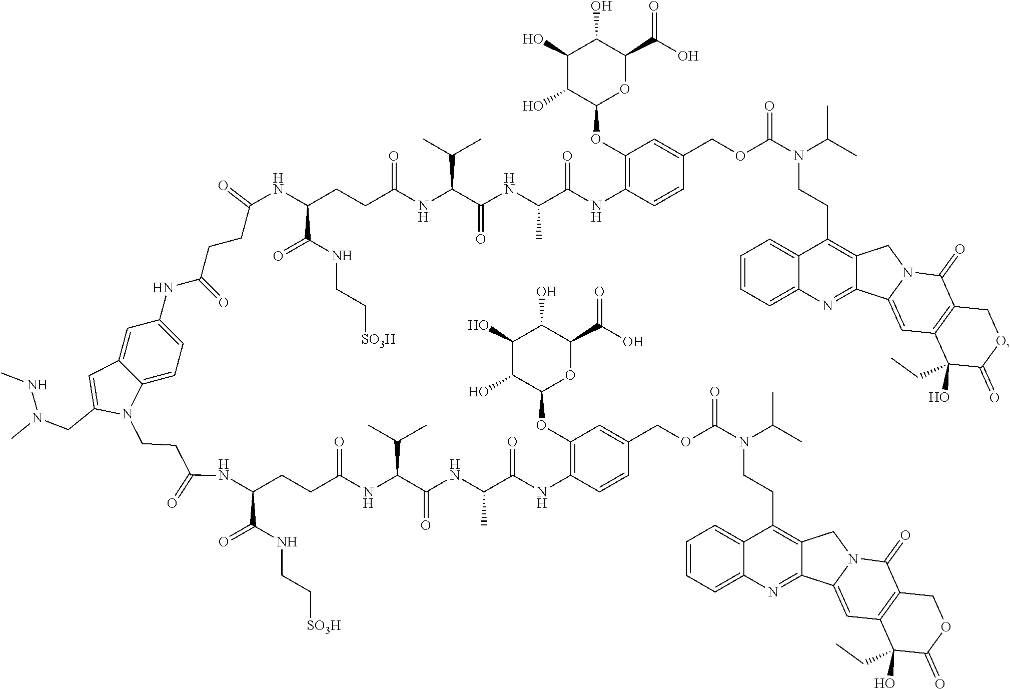 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CC[C@H](NC(=O)CCC(=O)Nc2ccc3c(c2)cc(CN(C)NC)n3CCC(=O)N[C@@H](CCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(=O)NCCS(=O)(=O)O)C(=O)NCCS(=O)(=O)O)C(C)C)c(O[C@@H]2O[C@H](C(=O)O)[C@@H](O)[C@H](O)[C@H]2O)c1)C(C)C]

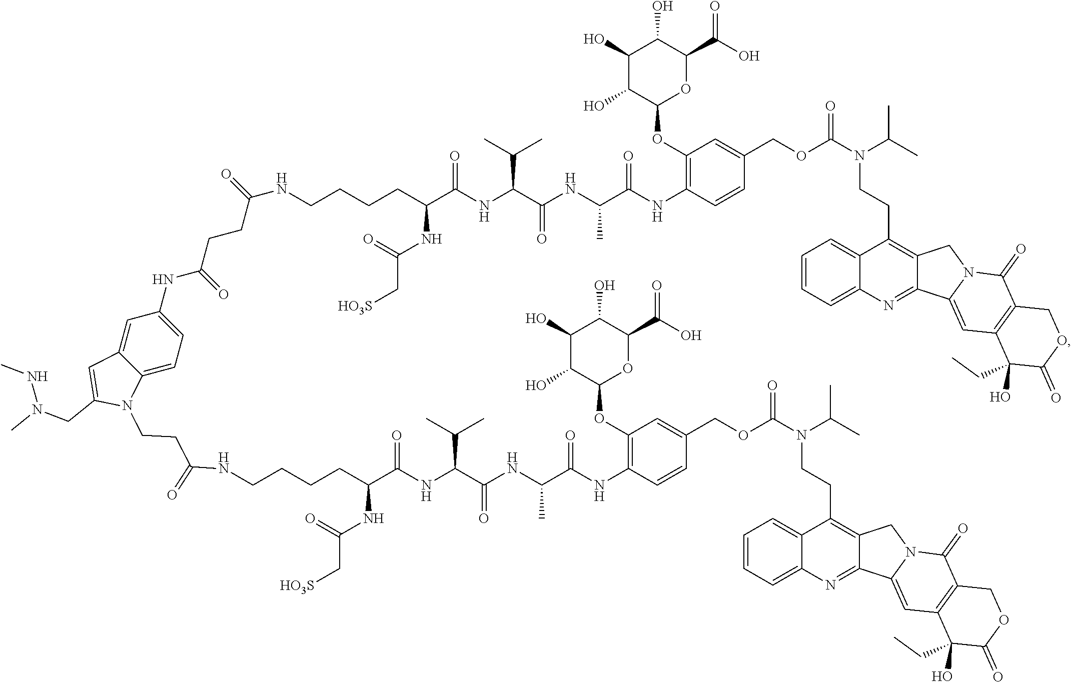 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CCCCNC(=O)CCC(=O)Nc2ccc3c(c2)cc(CN(C)NC)n3CCC(=O)NCCCC[C@H](NC(=O)CS(=O)(=O)O)C(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)NC(=O)CS(=O)(=O)O)C(C)C)c(O[C@@H]2O[C@H](C(=O)O)[C@@H](O)[C@H](O)[C@H]2O)c1)C(C)C]

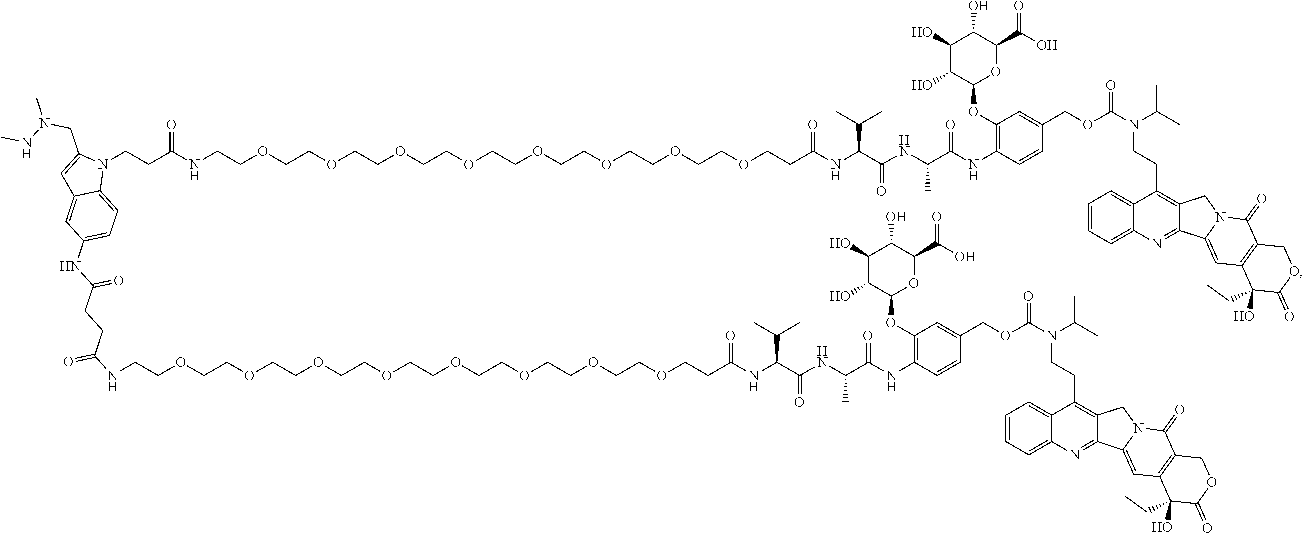 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCOCCOCCOCCOCCOCCOCCOCCOCCNC(=O)CCC(=O)Nc2ccc3c(c2)cc(CN(C)NC)n3CCC(=O)NCCOCCOCCOCCOCCOCCOCCOCCOCCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)c(O[C@@H]2O[C@H](C(=O)O)[C@@H](O)[C@H](O)[C@H]2O)c1)C(C)C]

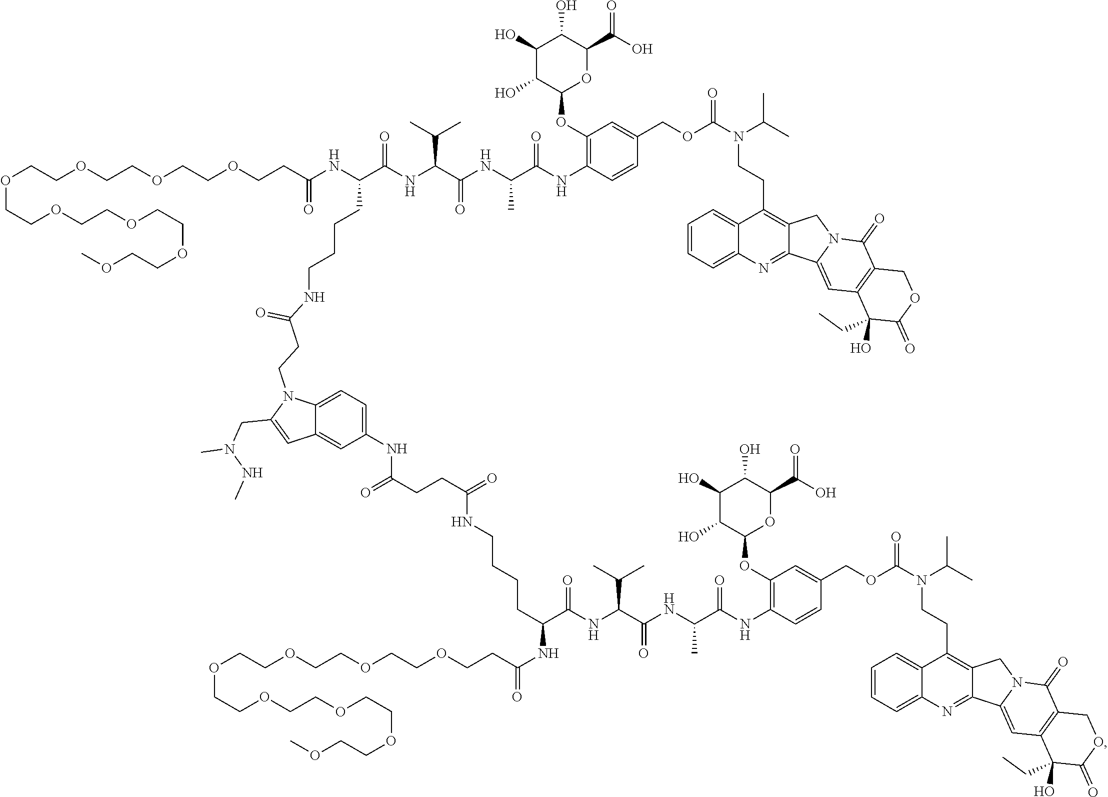 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CCCCNC(=O)CCC(=O)Nc2ccc3c(c2)cc(CN(C)NC)n3CCC(=O)NCCCC[C@H](NC(=O)CCOCCOCCOCCOCCOCCOCCOCCOC)C(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)NC(=O)CCOCCOCCOCCOCCOCCOCCOCCOC)C(C)C)c(O[C@@H]2O[C@H](C(=O)O)[C@@H](O)[C@H](O)[C@H]2O)c1)C(C)C]

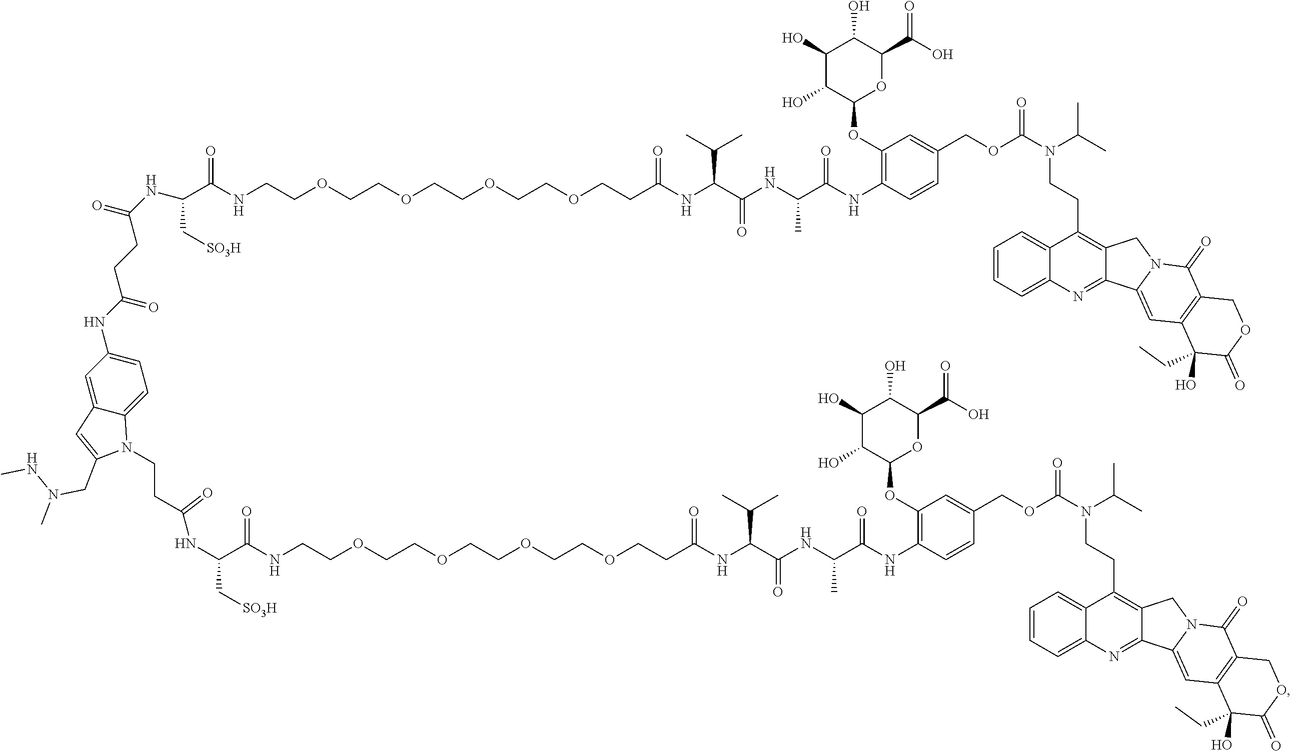 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCOCCOCCOCCOCCNC(=O)[C@H](CS(=O)(=O)O)NC(=O)CCC(=O)Nc2ccc3c(c2)cc(CN(C)NC)n3CCC(=O)N[C@@H](CS(=O)(=O)O)C(=O)NCCOCCOCCOCCOCCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)c(O[C@@H]2O[C@H](C(=O)O)[C@@H](O)[C@H](O)[C@H]2O)c1)C(C)C]

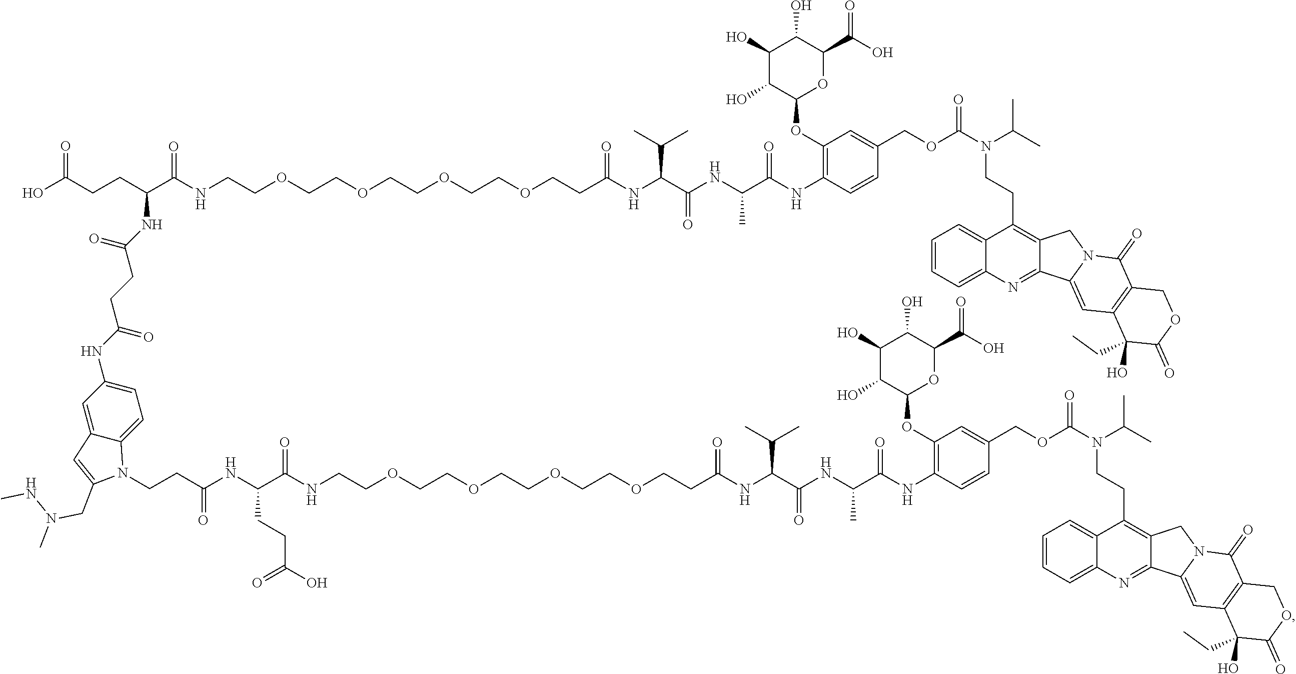 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCOCCOCCOCCOCCNC(=O)[C@H](CCC(=O)O)NC(=O)CCC(=O)Nc2ccc3c(c2)cc(CN(C)NC)n3CCC(=O)N[C@@H](CCC(=O)O)C(=O)NCCOCCOCCOCCOCCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)c(O[C@@H]2O[C@H](C(=O)O)[C@@H](O)[C@H](O)[C@H]2O)c1)C(C)C]

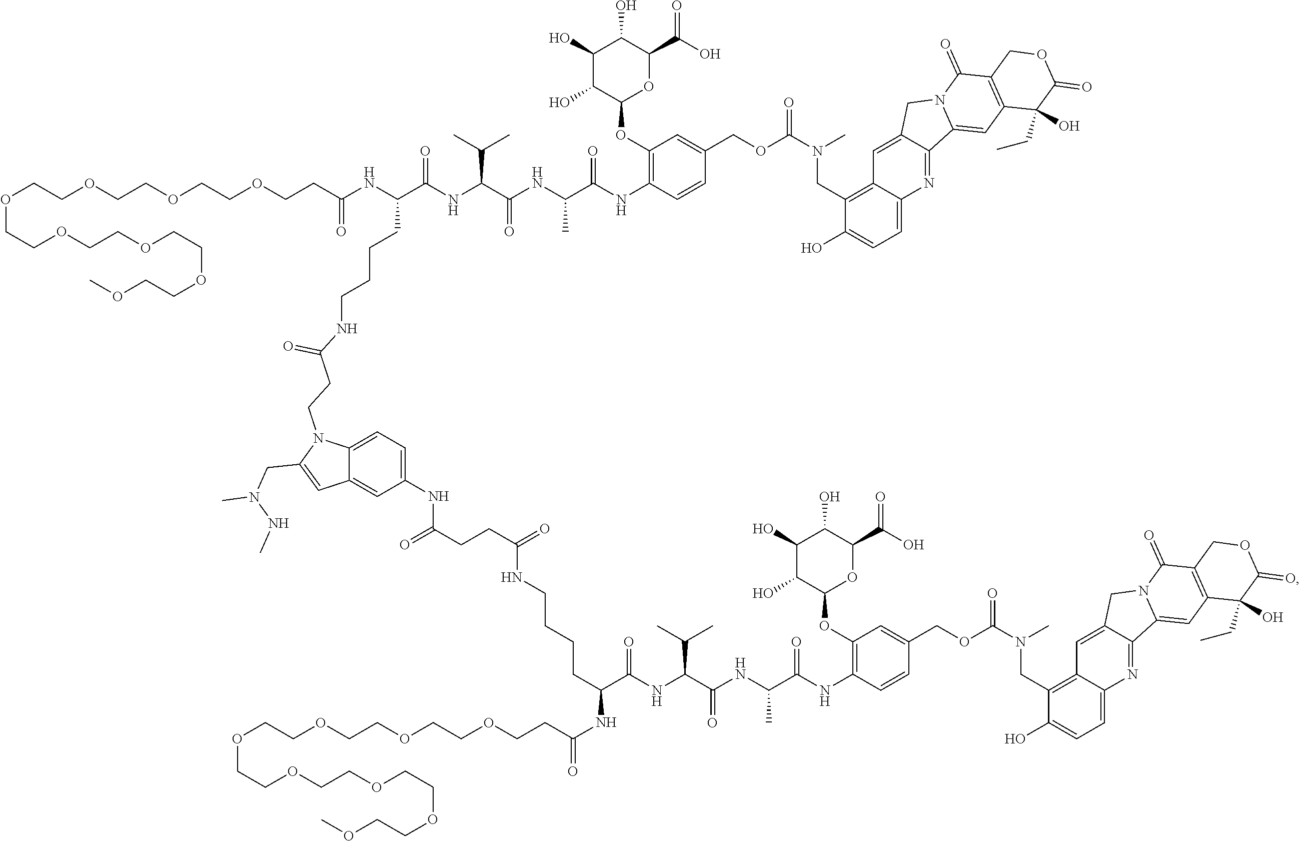 [SMILES: CC[C@@]1(O)C(=O)OCc2c1cc1n(c2=O)Cc2cc3c(CN(C)C(=O)OCc4ccc(NC(=O)[C@H](C)NC(=O)[C@@H](NC(=O)[C@H](CCCCNC(=O)CCC(=O)Nc5ccc6c(c5)cc(CN(C)NC)n6CCC(=O)NCCCC[C@H](NC(=O)CCOCCOCCOCCOCCOCCOCCOCCOC)C(=O)N[C@H](C(=O)N[C@@H](C)C(=O)Nc5ccc(COC(=O)N(C)Cc6c(O)ccc7nc8c(cc67)Cn6c-8cc7c(c6=O)COC(=O)[C@]7(O)CC)cc5O[C@@H]5O[C@H](C(=O)O)[C@@H](O)[C@H](O)[C@H]5O)C(C)C)NC(=O)CCOCCOCCOCCOCCOCCOCCOCCOC)C(C)C)c(O[C@@H]5O[C@H](C(=O)O)[C@@H](O)[C@H](O)[C@H]5O)c4)c(O)ccc3nc2-1]

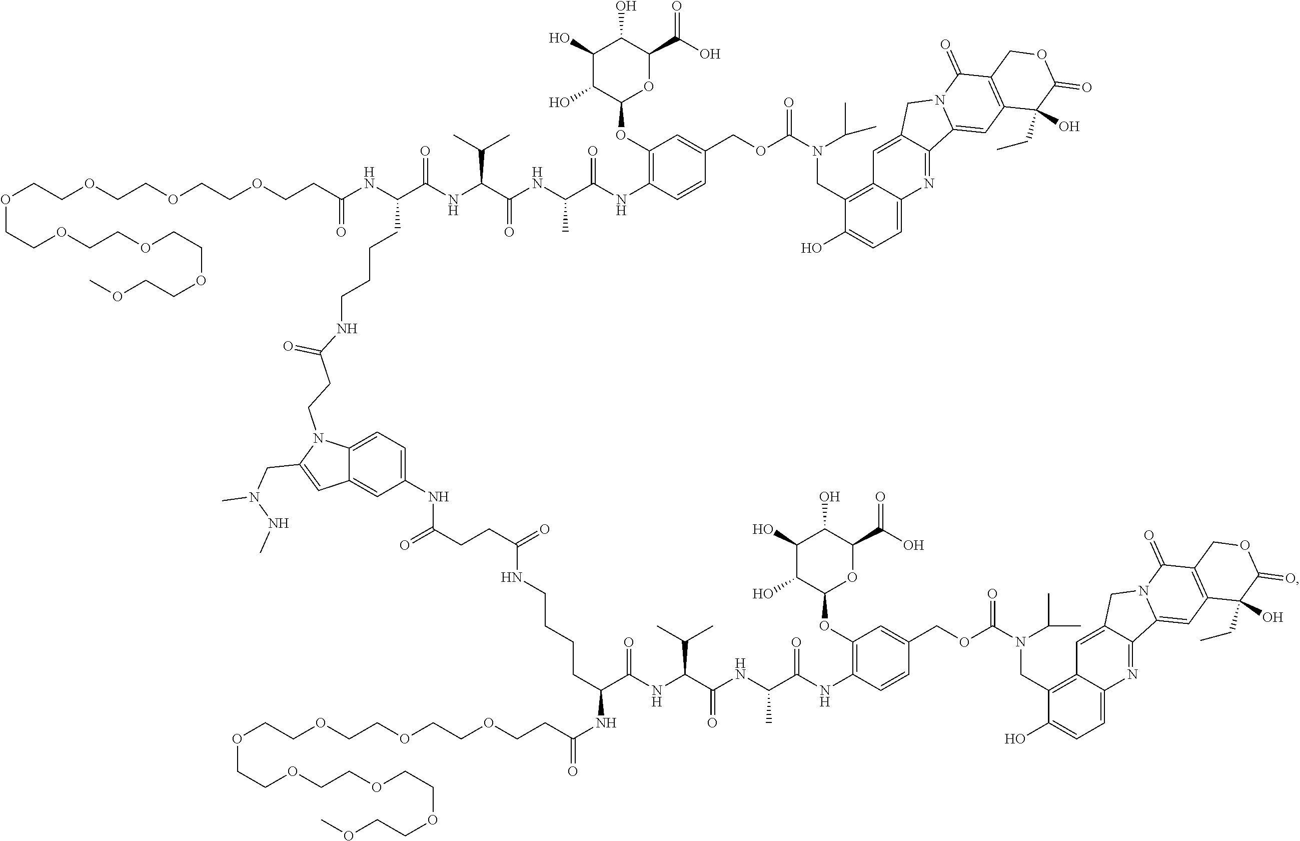 [SMILES: CC[C@@]1(O)C(=O)OCc2c1cc1n(c2=O)Cc2cc3c(CN(C(=O)OCc4ccc(NC(=O)[C@H](C)NC(=O)[C@@H](NC(=O)[C@H](CCCCNC(=O)CCC(=O)Nc5ccc6c(c5)cc(CN(C)NC)n6CCC(=O)NCCCC[C@H](NC(=O)CCOCCOCCOCCOCCOCCOCCOCCOC)C(=O)N[C@H](C(=O)N[C@@H](C)C(=O)Nc5ccc(COC(=O)N(Cc6c(O)ccc7nc8c(cc67)Cn6c-8cc7c(c6=O)COC(=O)[C@]7(O)CC)C(C)C)cc5O[C@@H]5O[C@H](C(=O)O)[C@@H](O)[C@H](O)[C@H]5O)C(C)C)NC(=O)CCOCCOCCOCCOCCOCCOCCOCCOC)C(C)C)c(O[C@@H]5O[C@H](C(=O)O)[C@@H](O)[C@H](O)[C@H]5O)c4)C(C)C)c(O)ccc3nc2-1]

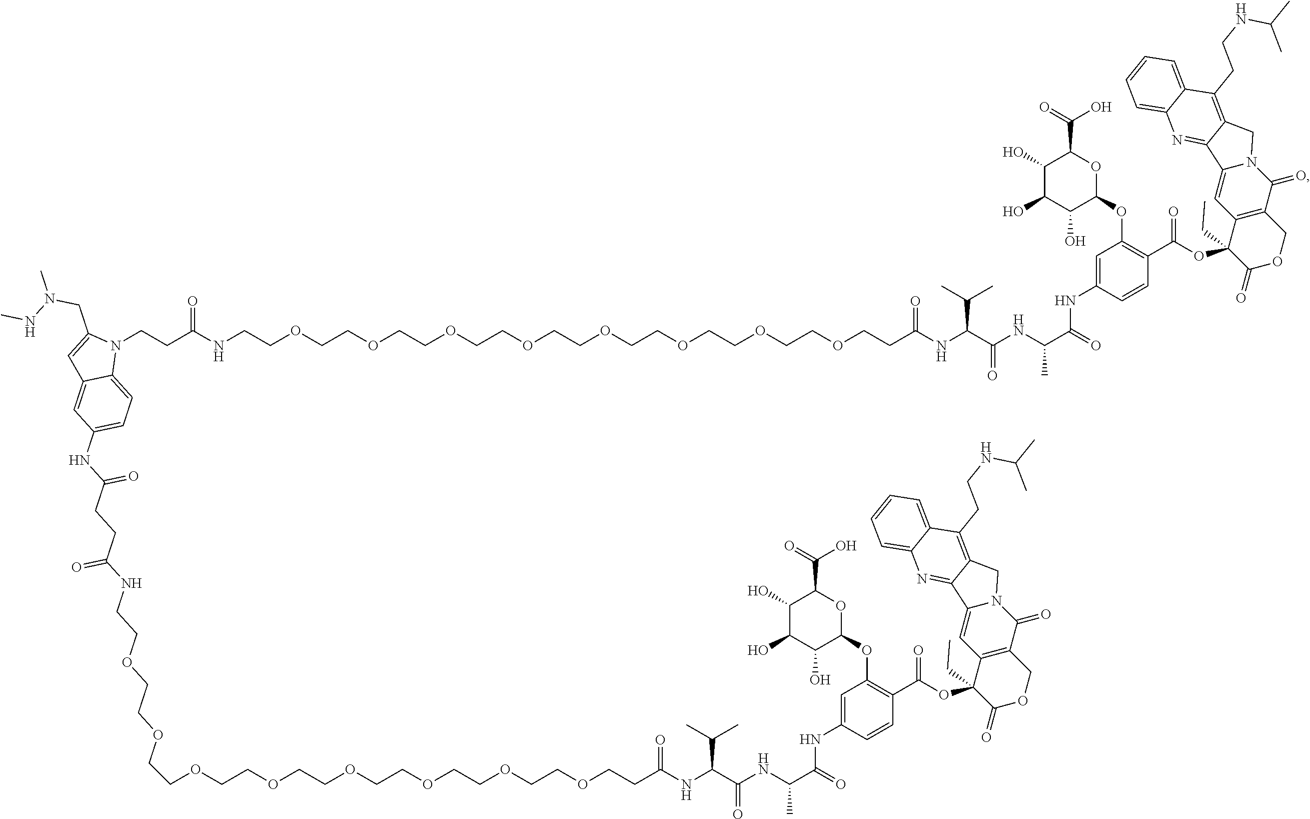 [SMILES: CC[C@@]1(OC(=O)c2ccc(NC(=O)[C@H](C)NC(=O)[C@@H](NC(=O)CCOCCOCCOCCOCCOCCOCCOCCOCCNC(=O)CCC(=O)Nc3ccc4c(c3)cc(CN(C)NC)n4CCC(=O)NCCOCCOCCOCCOCCOCCOCCOCCOCCC(=O)N[C@H](C(=O)N[C@@H](C)C(=O)Nc3ccc(C(=O)O[C@]4(CC)C(=O)OCc5c4cc4n(c5=O)Cc5c-4nc4ccccc4c5CCNC(C)C)c(O[C@@H]4O[C@H](C(=O)O)[C@@H](O)[C@H](O)[C@H]4O)c3)C(C)C)C(C)C)cc2O[C@@H]2O[C@H](C(=O)O)[C@@H](O)[C@H](O)[C@H]2O)C(=O)OCc2c1cc1n(c2=O)Cc2c-1nc1ccccc1c2CCNC(C)C]

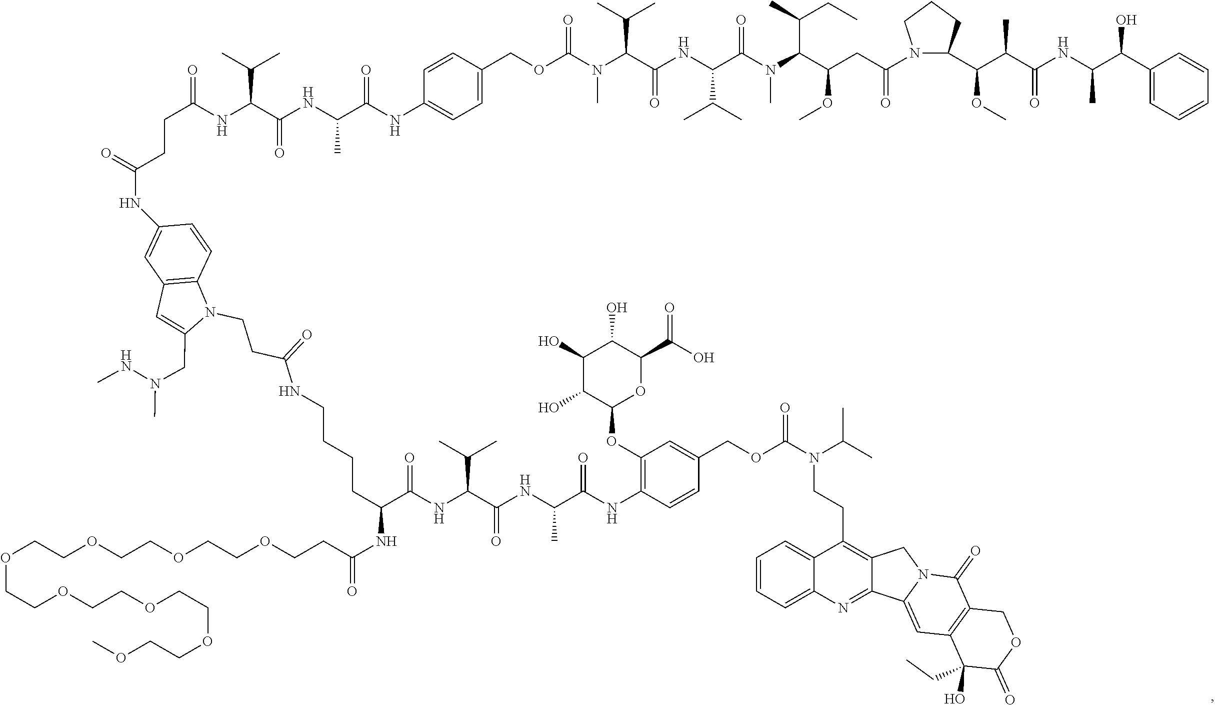 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCC(=O)Nc2ccc3c(c2)cc(CN(C)NC)n3CCC(=O)NCCCC[C@H](NC(=O)CCOCCOCCOCCOCCOCCOCCOCCOC)C(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)cc1)C(C)C]

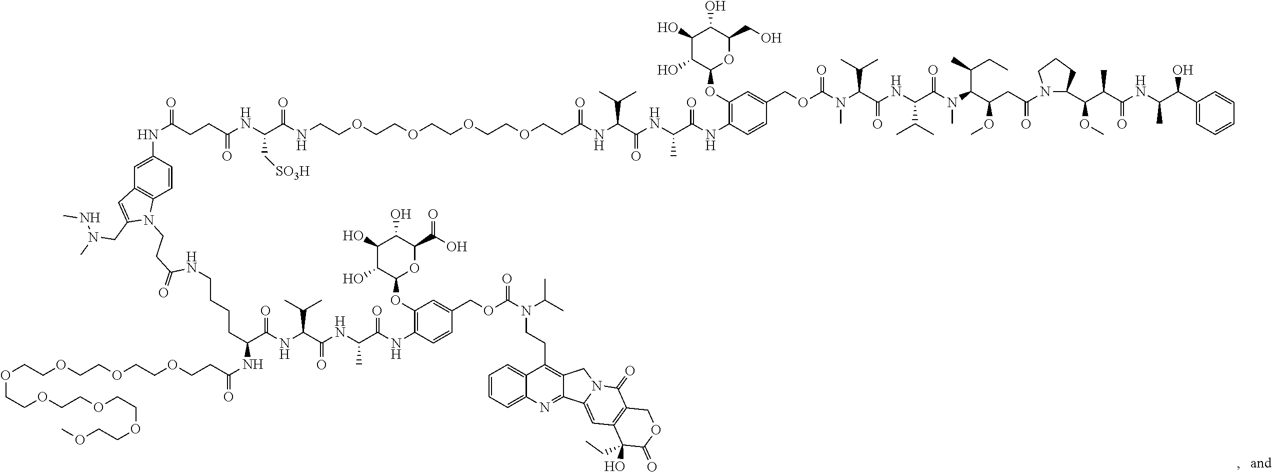

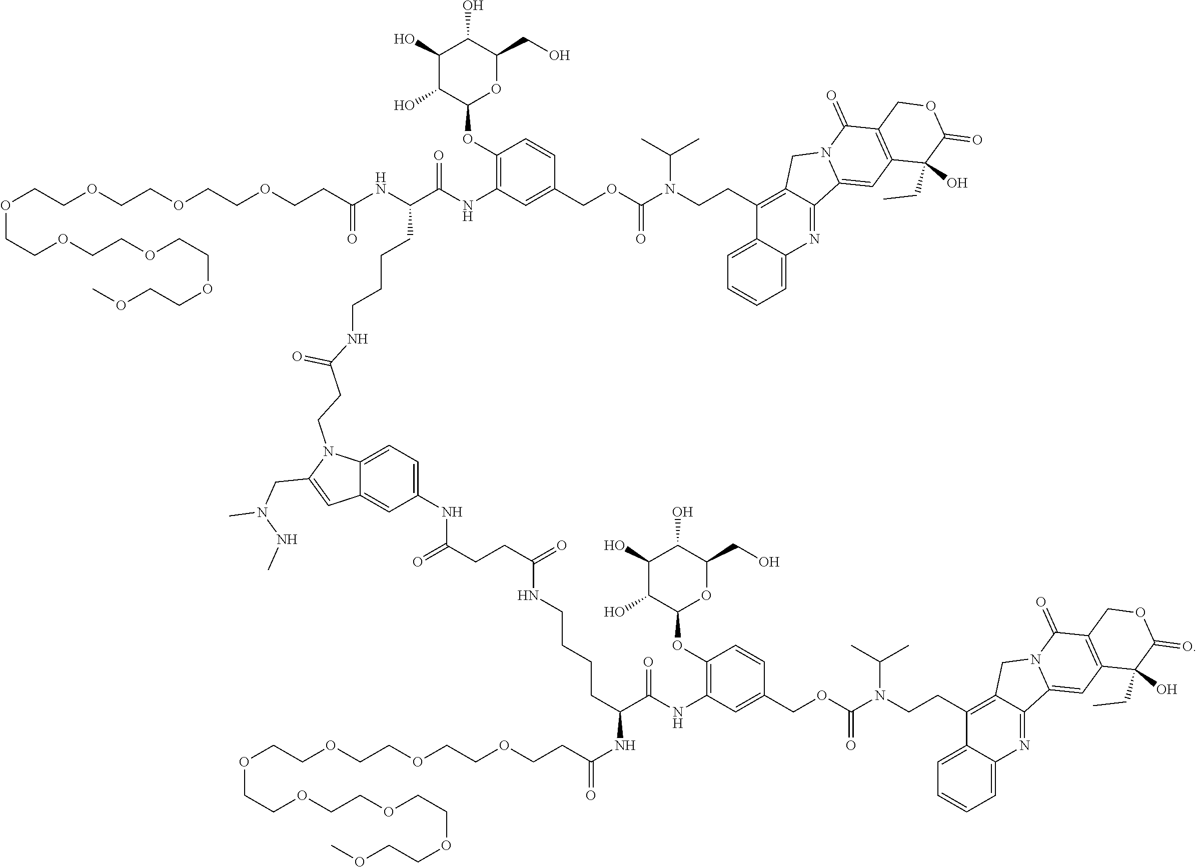 [SMILES: CC[C@@]1(O)C(=O)OCc2c1cc1n(c2=O)Cc2c-1nc1ccccc1c2CCN(C(=O)OCc1ccc(O[C@@H]2O[C@H](CO)[C@@H](O)[C@H](O)[C@H]2O)c(NC(=O)[C@H](CCCCNC(=O)CCC(=O)Nc2ccc3c(c2)cc(CN(C)NC)n3CCC(=O)NCCCC[C@H](NC(=O)CCOCCOCCOCCOCCOCCOCCOCCOC)C(=O)Nc2cc(COC(=O)N(CCc3c4c(nc5ccccc35)-c3cc5c(c(=O)n3C4)COC(=O)[C@]5(O)CC)C(C)C)ccc2O[C@@H]2O[C@H](CO)[C@@H](O)[C@H](O)[C@H]2O)NC(=O)CCOCCOCCOCCOCCOCCOCCOCCOC)c1)C(C)C]

In some embodiments, the compound is selected from:

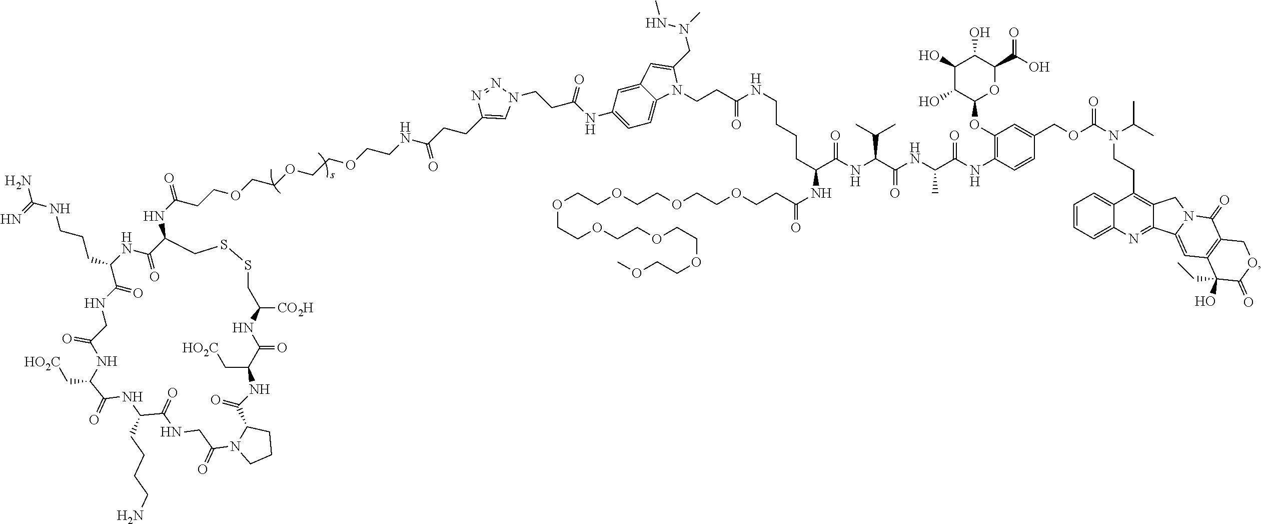

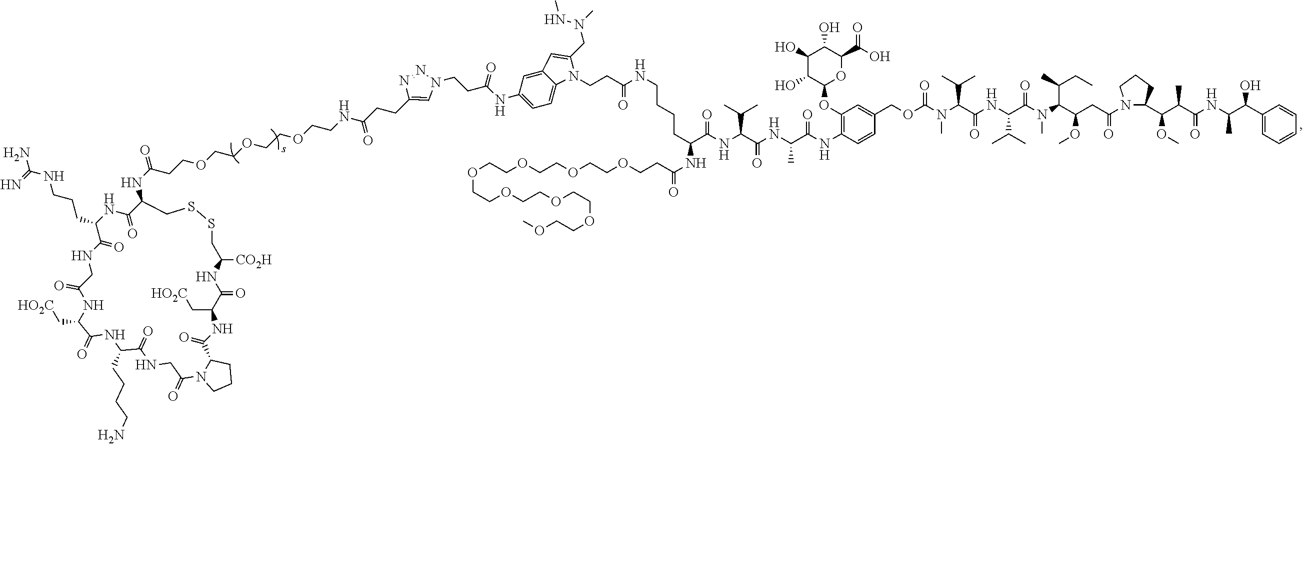

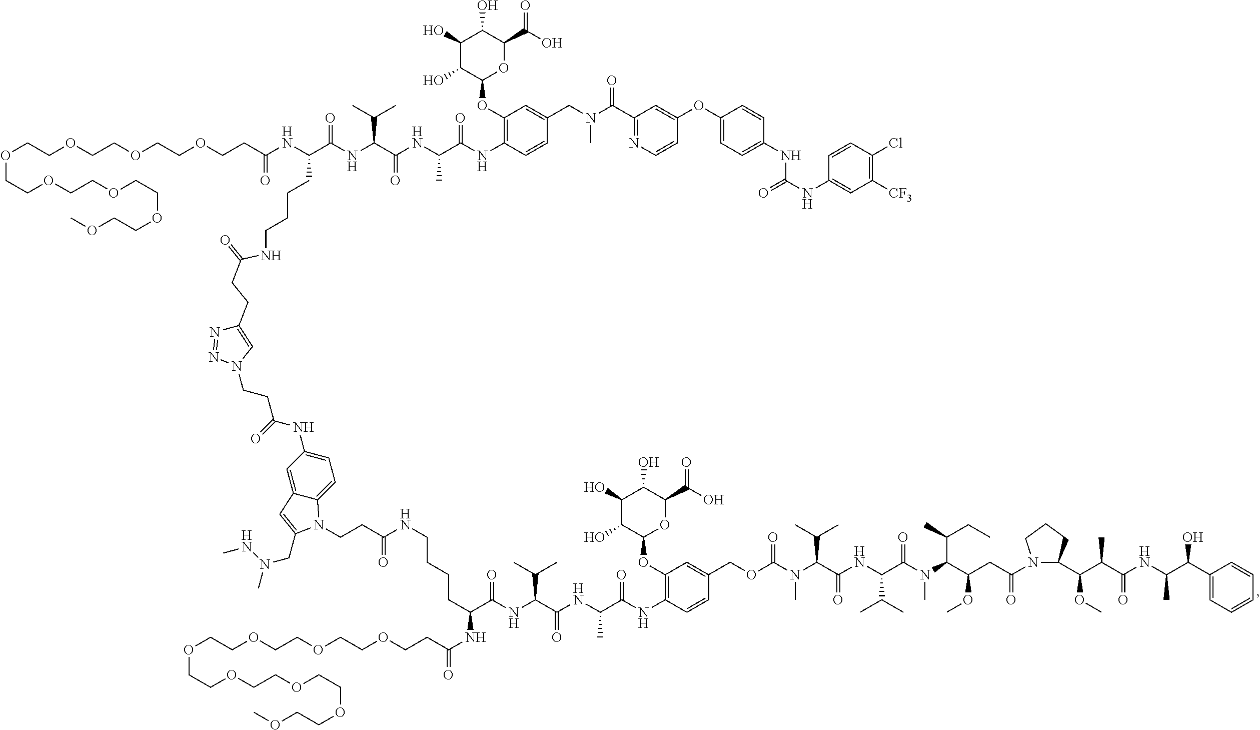

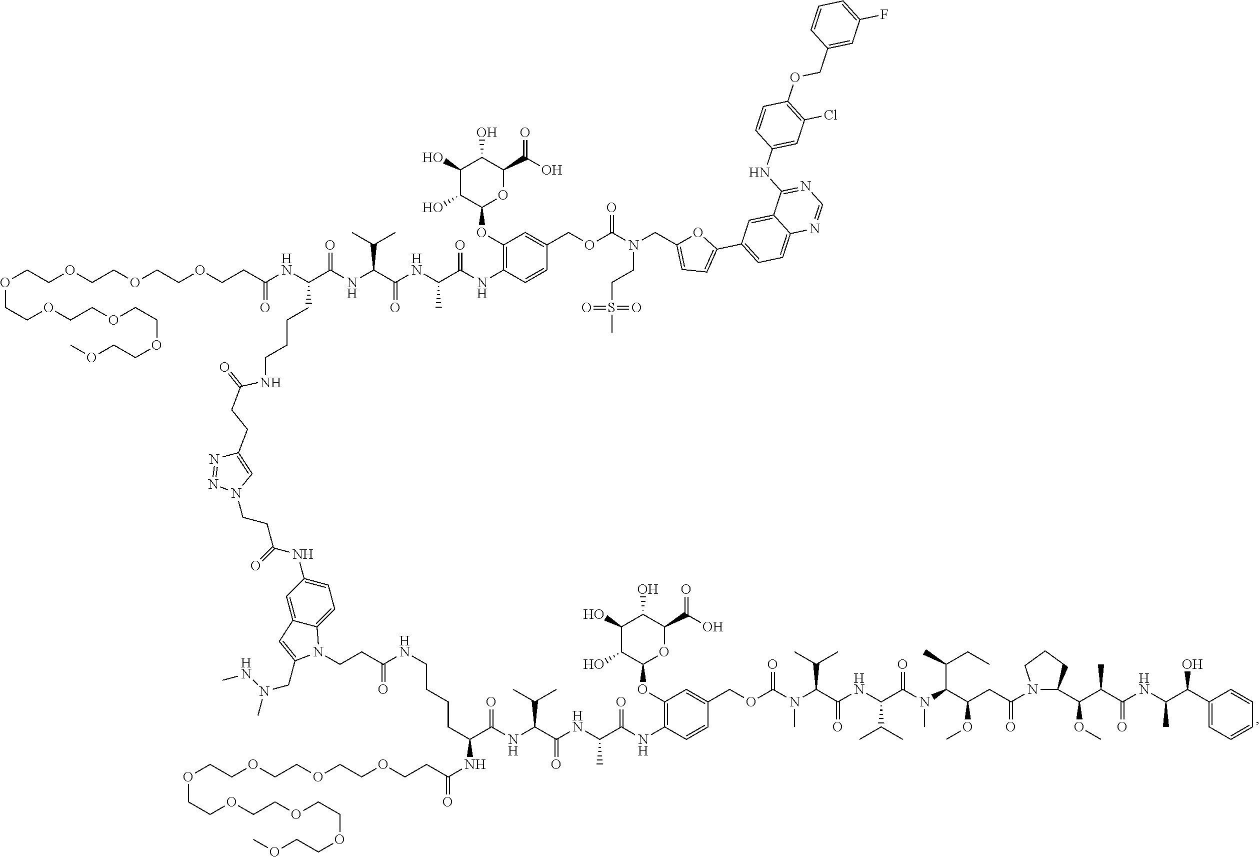

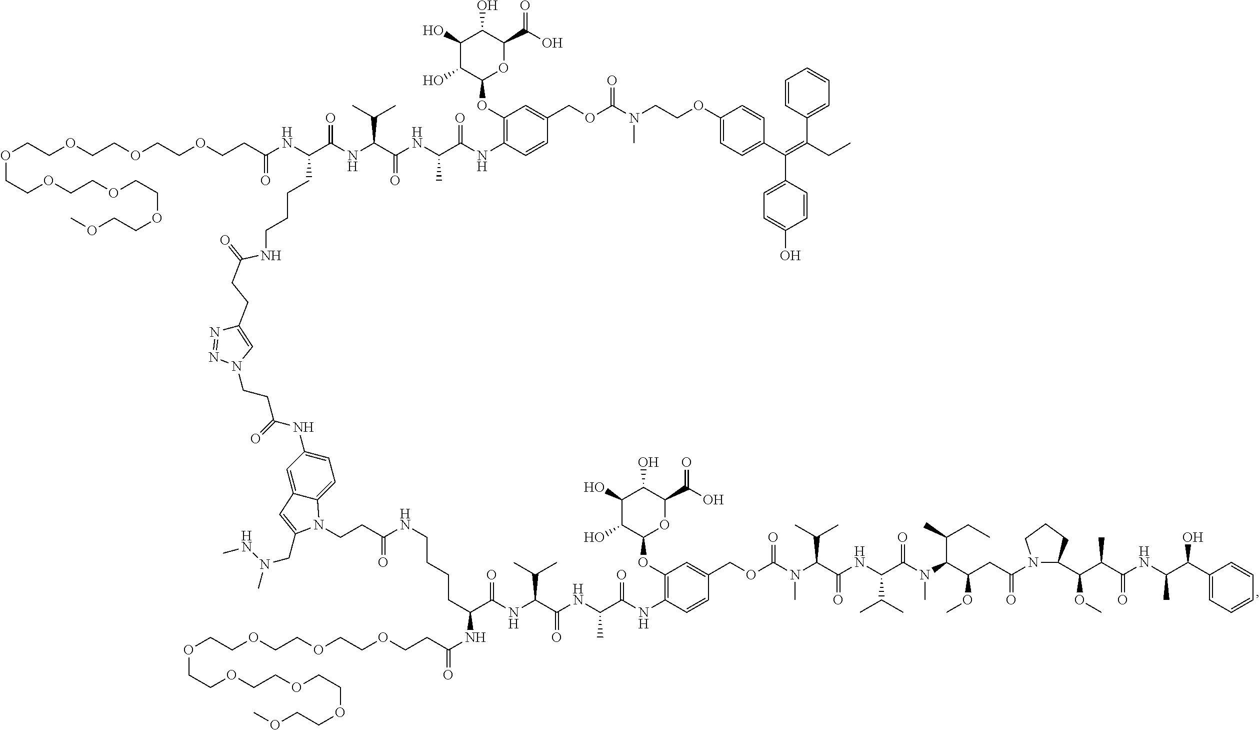

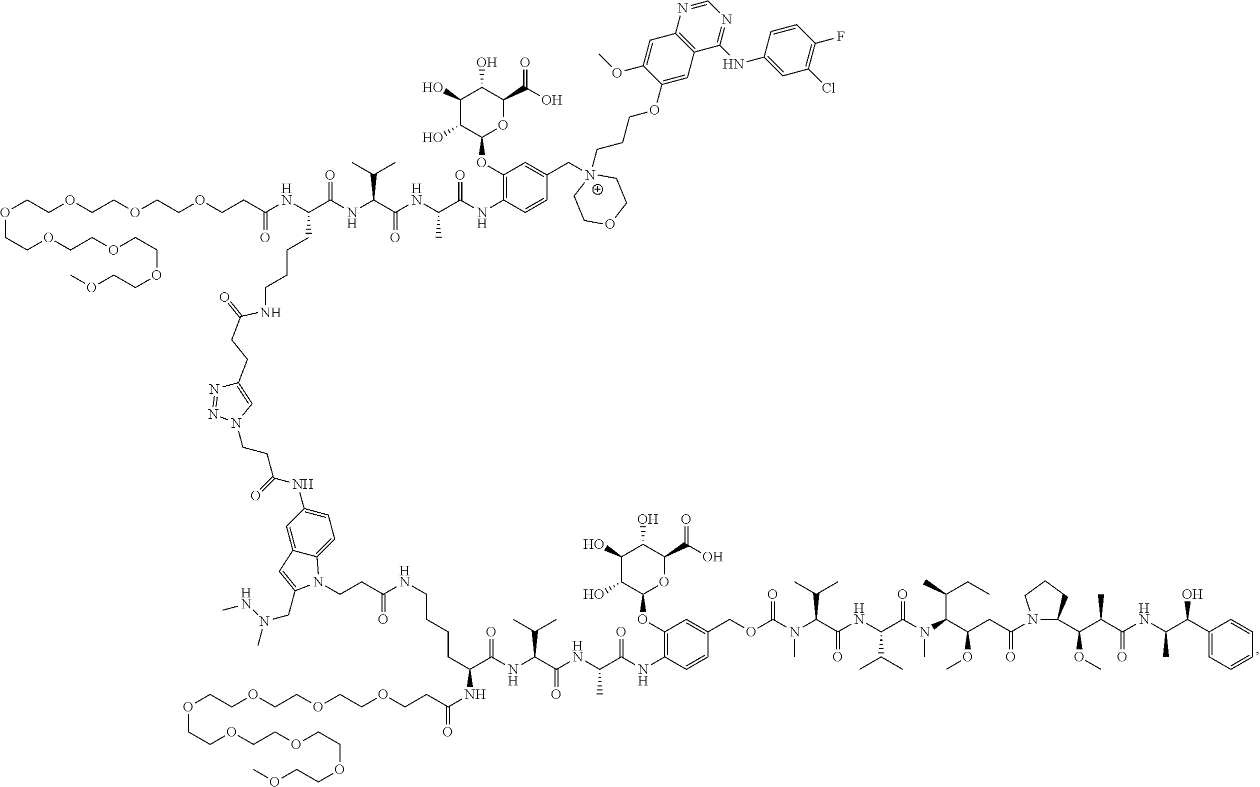

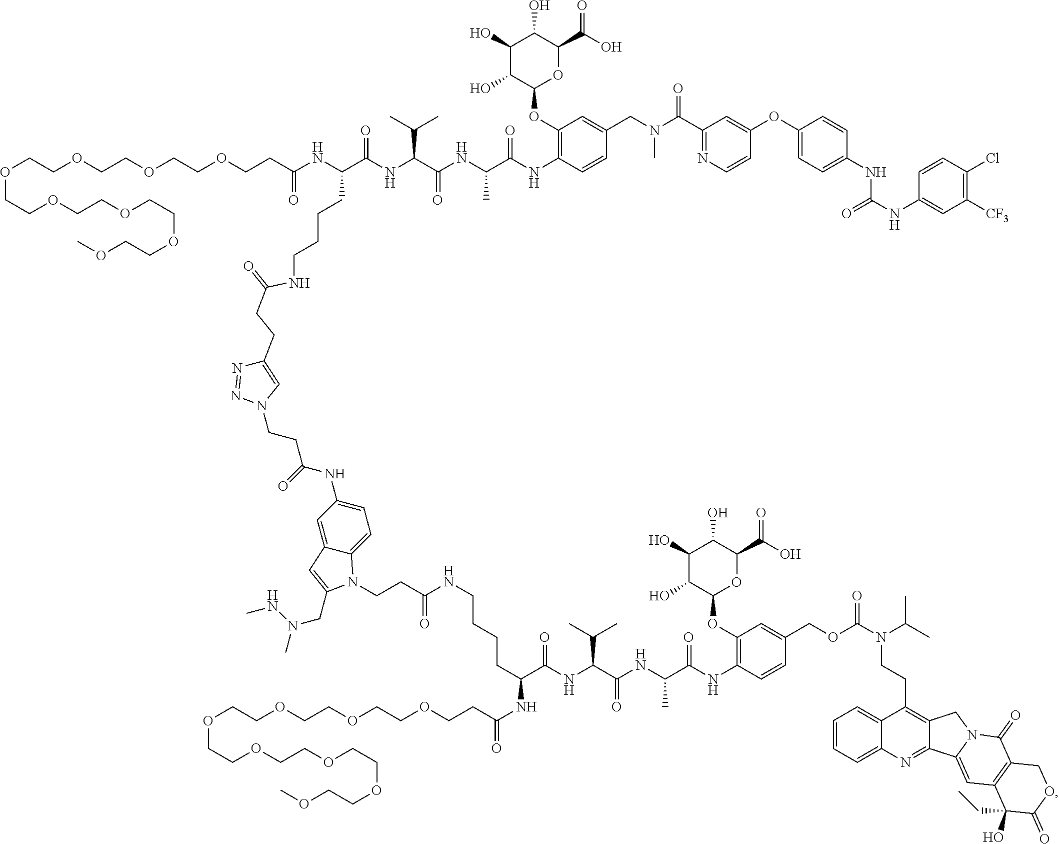 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CCCCNC(=O)CCn2c(CN(C)NC)cc3cc(NC(=O)CCn4cc(CCC(=O)NCCCC[C@H](NC(=O)CCOCCOCCOCCOCCOCCOCCOCCOC)C(=O)N[C@H](C(=O)N[C@@H](C)C(=O)Nc5ccc(CN(C)C(=O)c6cc(Oc7ccc(NC(=O)Nc8ccc(Cl)c(C(F)(F)F)c8)cc7)ccn6)cc5O[C@@H]5O[C@H](C(=O)O)[C@@H](O)[C@H](O)[C@H]5O)C(C)C)nn4)ccc32)NC(=O)CCOCCOCCOCCOCCOCCOCCOCCOC)C(C)C)c(O[C@@H]2O[C@H](C(=O)O)[C@@H](O)[C@H](O)[C@H]2O)c1)C(C)C]

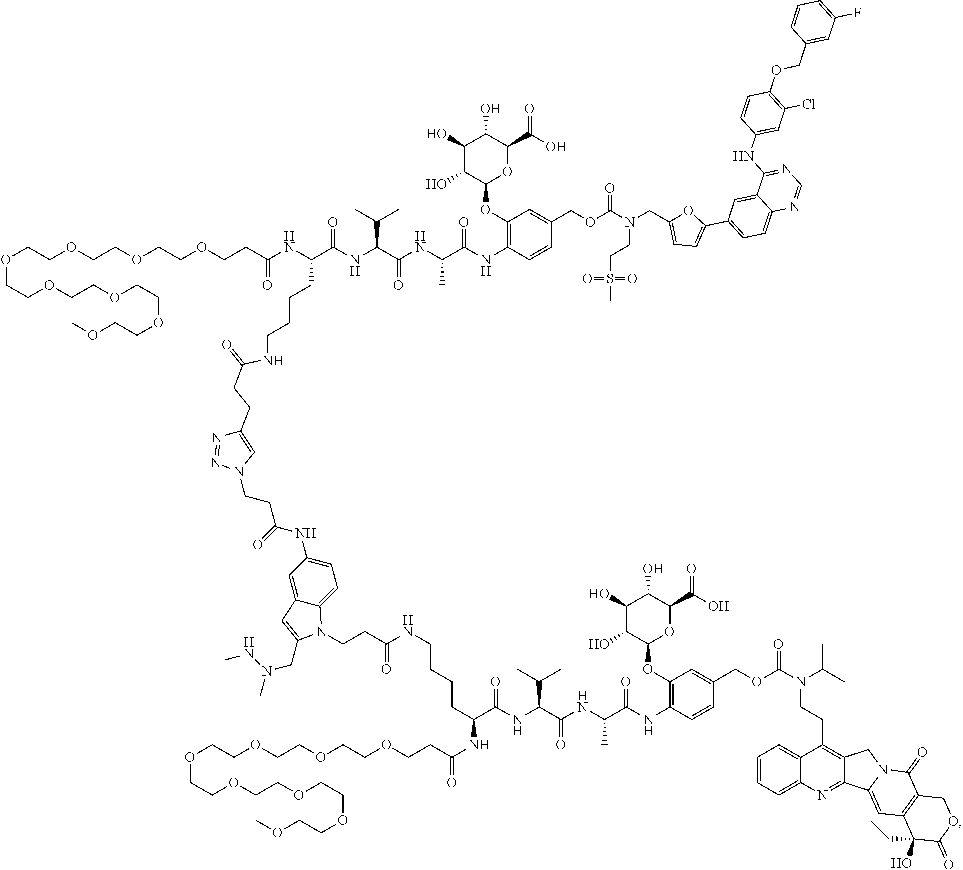 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CCCCNC(=O)CCn2c(CN(C)NC)cc3cc(NC(=O)CCn4cc(CCC(=O)NCCCC[C@H](NC(=O)CCOCCOCCOCCOCCOCCOCCOCCOC)C(=O)N[C@H](C(=O)N[C@@H](C)C(=O)Nc5ccc(COC(=O)N(CCS(C)(=O)=O)Cc6ccc(-c7ccc8ncnc(Nc9ccc(OCc%10cccc(F)c%10)c(Cl)c9)c8c7)o6)cc5O[C@@H]5O[C@H](C(=O)O)[C@@H](O)[C@H](O)[C@H]5O)C(C)C)nn4)ccc32)NC(=O)CCOCCOCCOCCOCCOCCOCCOCCOC)C(C)C)c(O[C@@H]2O[C@H](C(=O)O)[C@@H](O)[C@H](O)[C@H]2O)c1)C(C)C]

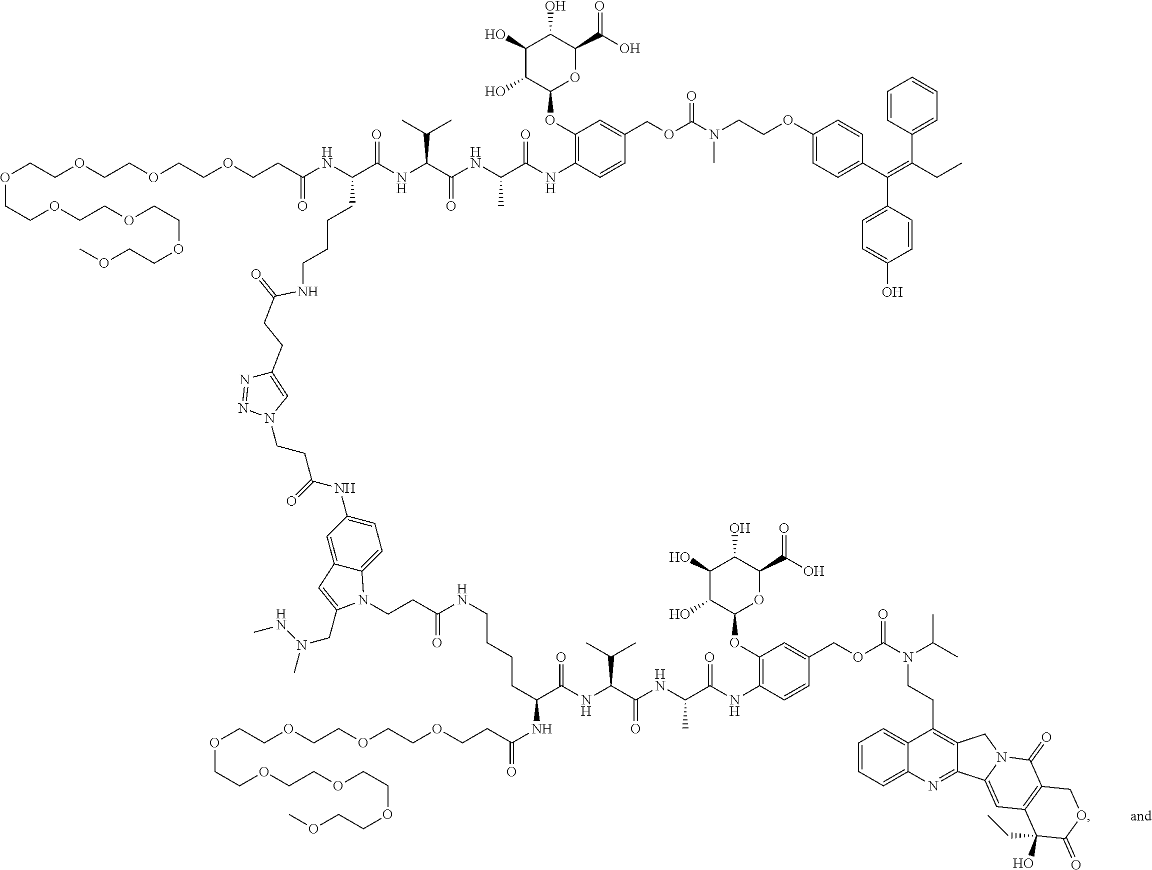 [SMILES: CC/C(=C(\c1ccc(O)cc1)c1ccc(OCCN(C)C(=O)OCc2ccc(NC(=O)[C@H](C)NC(=O)[C@@H](NC(=O)[C@H](CCCCNC(=O)CCc3cn(CCC(=O)Nc4ccc5c(c4)cc(CN(C)NC)n5CCC(=O)NCCCC[C@H](NC(=O)CCOCCOCCOCCOCCOCCOCCOCCOC)C(=O)N[C@H](C(=O)N[C@@H](C)C(=O)Nc4ccc(COC(=O)N(CCc5c6c(nc7ccccc57)-c5cc7c(c(=O)n5C6)COC(=O)[C@]7(O)CC)C(C)C)cc4O[C@@H]4O[C@H](C(=O)O)[C@@H](O)[C@H](O)[C@H]4O)C(C)C)nn3)NC(=O)CCOCCOCCOCCOCCOCCOCCOCCOC)C(C)C)c(O[C@@H]3O[C@H](C(=O)O)[C@@H](O)[C@H](O)[C@H]3O)c2)cc1)c1ccccc1 ; "and"]

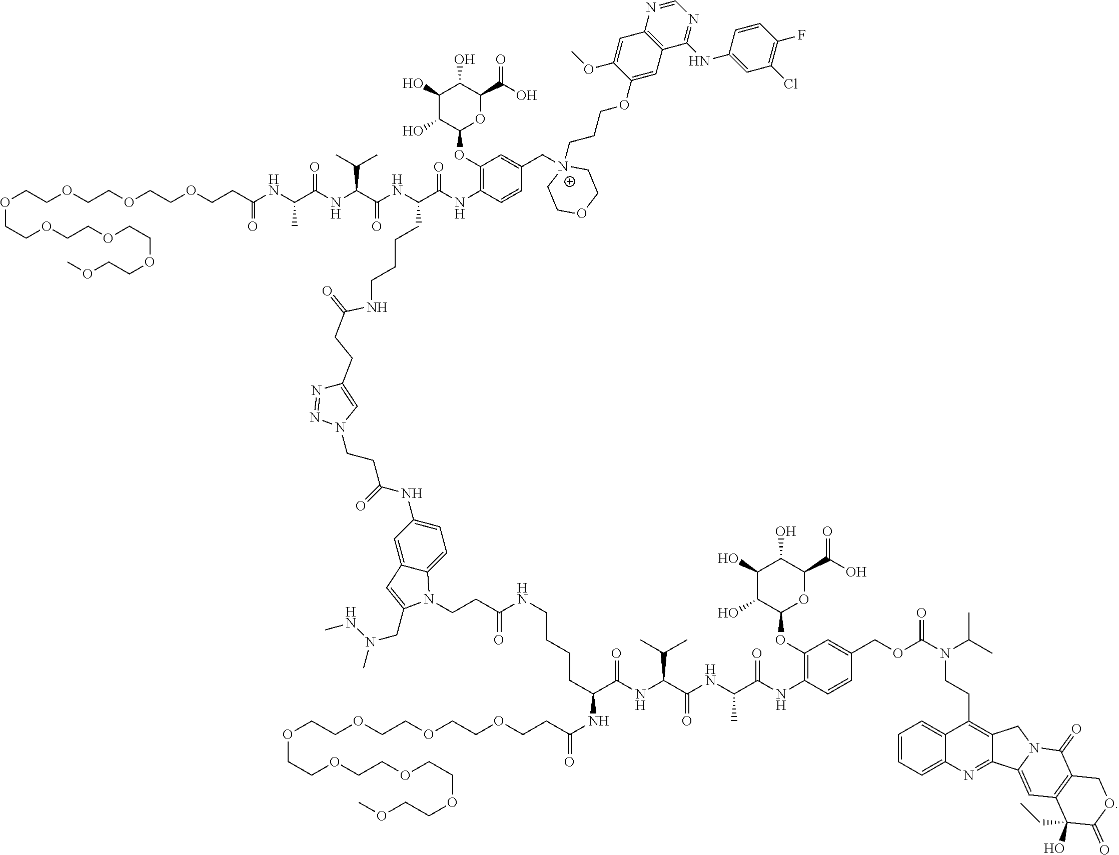 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CCCCNC(=O)CCn2c(CN(C)NC)cc3cc(NC(=O)CCn4cc(CCC(=O)NCCCC[C@H](NC(=O)[C@@H](NC(=O)[C@H](C)NC(=O)CCOCCOCCOCCOCCOCCOCCOCCOC)C(C)C)C(=O)Nc5ccc(C[N+]6(CCCOc7cc8c(Nc9ccc(F)c(Cl)c9)ncnc8cc7OC)CCOCC6)cc5O[C@@H]5O[C@H](C(=O)O)[C@@H](O)[C@H](O)[C@H]5O)nn4)ccc32)NC(=O)CCOCCOCCOCCOCCOCCOCCOCCOC)C(C)C)c(O[C@@H]2O[C@H](C(=O)O)[C@@H](O)[C@H](O)[C@H]2O)c1)C(C)C]

Aspects of the present disclosure include a pharmaceutical composition comprising a conjugate as described herein, and a pharmaceutically-acceptable excipient.

Aspects of the present disclosure include a method of administering a conjugate to a subject, where the method includes administering to a subject a conjugate as described herein.

Aspects of the present disclosure include a method of treating cancer, where the method includes administering to a subject a therapeutically effective amount of a conjugate as described herein, where the administering is effective to treat the cancer in the subject.

### BRIEF DESCRIPTION OF THE DRAWINGS

FIG. 1 shows a schematic drawing of the HIPS ligation for the synthesis of ADCs. Antibodies carrying aldehyde moieties are reacted with a Hydrazino-iso-Pictet-Spengler (HIPS) linker and payload to generate a site-specifically conjugated ADC with a stable azacarboline linkage.

FIG. 2 shows a schematic representation of branched HIPS ligation for the synthesis of ADCs. Antibodies carrying four aldehyde moieties are reacted with a branched HIPS linker to generate ADCs with drug-to-antibody (DAR) value of up to 8, according to embodiments of the present disclosure.

FIG. 3 shows a graph indicating Construct 15 CT-tagged polatuzumab conjugate yielded a DAR of 3.57 as determined by hydrophobic interaction chromatography (HIC), according to embodiments of the present disclosure.

FIG. 4 shows a graph indicating Construct 15 CT-tagged polatuzumab conjugate was 97.5% monomeric as determined by analytical size-exclusion chromatography (SEC), according to embodiments of the present disclosure.

FIG. 5 shows a graph indicating Construct 15 CH1-3/CT-tagged polatuzumab conjugate yielded a DAR of 7.19 as determined by polymeric reversed phase (PLRP), according to embodiments of the present disclosure.

FIG. 6 shows a graph indicating Construct 15 CH1-3/CT-tagged polatuzumab conjugate was 97.7% monomeric as determined by analytical SEC, according to embodiments of the present disclosure.

FIG. 7 shows a graph indicating Construct 15 LC-9/CT double-tagged polatuzumab conjugate yielded a DAR of 7.06 as determined by PLRP, according to embodiments of the present disclosure.

FIG. 8 shows a graph indicating Construct 15 LC-9/CT double-tagged polatuzumab conjugate was 99.1% monomeric as determined by analytical SEC, according to embodiments of the present disclosure.

FIG. 9 shows a graph indicating Construct 15 LC-9/CH2-3 double-tagged polatuzumab conjugate yielded a DAR of 7.36 as determined by PLRP, according to embodiments of the present disclosure.

FIG. 10 shows a graph indicating Construct 15 LC-9/CH2-3 double-tagged polatuzumab conjugate was 87.3% monomeric as determined by analytical SEC, according to embodiments of the present disclosure.

FIG. 11 shows a graph indicating Construct 15 CH1-5/CT double-tagged polatuzumab conjugate yielded a DAR of 5.78 as determined by PLRP, according to embodiments of the present disclosure.

FIG. 12 shows a graph indicating Construct 15 CH1-5/CT double-tagged polatuzumab conjugate was 99.7% monomeric as determined by analytical SEC, according to embodiments of the present disclosure.

FIG. 13 shows a graph indicating Construct 15 CH1-6/CT double-tagged polatuzumab conjugate yielded a DAR of 6.86 as determined by PLRP, according to embodiments of the present disclosure.

FIG. 14 shows a graph indicating Construct 15 CH1-6/CT double-tagged polatuzumab conjugate was 99.7% monomeric as determined by analytical SEC, according to embodiments of the present disclosure.

FIG. 15 shows a graph indicating Construct 15 CH1-5/CH2-3 double-tagged polatuzumab conjugate yielded a DAR of 7.06 as determined by PLRP, according to embodiments of the present disclosure.

FIG. 16 shows a graph indicating Construct 15 CH1-5/CH2-3 double-tagged polatuzumab conjugate was 92.8% monomeric as determined by analytical SEC, according to embodiments of the present disclosure.

FIG. 17 shows a graph indicating Construct 15 CH1-6/CH2-3 double-tagged polatuzumab conjugate yielded a DAR of 7.14 as determined by PLRP, according to embodiments of the present disclosure.

FIG. 18 shows a graph indicating Construct 15 CH1-6/CH2-3 double-tagged polatuzumab conjugate was 98.5% monomeric as determined by analytical SEC, according to embodiments of the present disclosure.

FIG. 19 shows a graph indicating Construct 22 CH1-3/CT double-tagged polatuzumab conjugate yielded a DAR of 3.87 as determined by PLRP, according to embodiments of the present disclosure.

FIG. 20 shows a graph indicating Construct 22 CH1-3/CT double-tagged polatuzumab conjugate was 93.6% monomeric as determined by analytical SEC, according to embodiments of the present disclosure.

FIG. 21 shows a graph indicating Construct 22 CT-tagged polatuzumab conjugate yielded a DAR of 1.91 as determined by HIC, according to embodiments of the present disclosure.

FIG. 22 shows a graph indicating Construct 22 CT-tagged polatuzumab conjugate is 95.0% monomeric was determined by analytical SEC, according to embodiments of the present disclosure.

FIG. 23 shows a graph indicating Construct 25 CH1-3/CT-tagged polatuzumab conjugate yielded a DAR of 4.66 as determined by PLRP, according to embodiments of the present disclosure.

FIG. 24 shows a graph indicating Construct 25 CH1-3/CT-tagged polatuzumab conjugate was 96.9% monomeric as determined by analytical SEC, according to embodiments of the present disclosure.

FIG. 25 shows a graph indicating Construct 25 CT-tagged polatuzumab conjugate yielded a DAR of 2.27 as determined by HIC, according to embodiments of the present disclosure.

FIG. 26 shows a graph indicating Construct 25 CT-tagged polatuzumab conjugate was 96.7% monomeric as determined by analytical SEC, according to embodiments of the present disclosure.

FIG. 27 shows a graph indicating Construct 18 CT-tagged polatuzumab conjugate yielded a DAR of 3.58 as determined by HIC, according to embodiments of the present disclosure.

FIG. 28 shows a graph indicating Construct 18 CT-tagged polatuzumab conjugate was 96.7% monomeric as determined by analytical SEC, according to embodiments of the present disclosure.

FIG. 29 shows a graph indicating Construct 15 CH1-3/CT-tagged anti-FITC conjugate yielded a DAR of 7.0 as determined by PLRP, according to embodiments of the present disclosure.

FIG. 30 shows a graph indicating Construct 15 CH1-3/CT-tagged anti-FITC conjugate was 98.1% monomeric as determined by analytical SEC, according to embodiments of the present disclosure.

FIG. 31 shows a graph indicating Construct 15 CH1-3/CT-tagged daclizumab conjugate yielded a DAR of 7.07 as determined by PLRP, according to embodiments of the present disclosure.

FIG. 32 shows a graph indicating Construct 15 CH1-3/CT-tagged daclizumab conjugate was 98.6% monomeric as determined by analytical SEC, according to embodiments of the present disclosure.

FIG. 33 shows a graph indicating Construct 15 CH1-3/CT-tagged anti-GPC-3 conjugate yielded a DAR of 6.86 as determined by PLRP, according to embodiments of the present disclosure.

FIG. 34 shows a graph indicating Construct 15 CH1-3/CT-tagged anti-GPC-3 conjugate was 98.2% monomeric as determined by analytical SEC, according to embodiments of the present disclosure.

FIG. 35 shows a graph indicating Construct 15 CH2-3/CT-tagged anti-FITC conjugate yielded a DAR of 4.60 as determined by PLRP, according to embodiments of the present disclosure.

FIG. 36 shows a graph indicating Construct 15 CH2-3/CT-tagged anti-FITC conjugate was 96.7% monomeric as determined by analytical SEC, according to embodiments of the present disclosure.

FIG. 37 shows a graph indicating Construct 15 CH1-3/CT-tagged alemtuzumab conjugate yielded a DAR of 7.07 as determined by PLRP, according to embodiments of the present disclosure.

FIG. 38 shows a graph indicating Construct 15 CH1-3/CT-tagged alemtuzumab conjugate was 98.0% monomeric as determined by analytical SEC, according to embodiments of the present disclosure.

FIG. 39 shows a graph indicating Construct 15 CH1-3/CT-tagged daclizumab conjugate yielded a DAR of 7.24 as determined by PLRP, according to embodiments of the present disclosure.

FIG. 40 shows a graph indicating Construct 15 CH1-3/CT-tagged daclizumab conjugate was 98.9% monomeric as determined by analytical SEC, according to embodiments of the present disclosure.

FIG. 41 shows a graph indicating Construct 15 CH1-3/CT-tagged anti-CD70 conjugate yielded a DAR of 6.97 as determined by PLRP, according to embodiments of the present disclosure.

FIG. 42 shows a graph indicating Construct 15 CH1-3/CT-tagged anti-CD70 conjugate was 99.4% monomeric as determined by analytical SEC, according to embodiments of the present disclosure.

FIG. 43 shows a graph indicating Construct 15 CH1-3/CT-tagged anti-nectin-4 conjugate yielded a DAR of 7.56 as determined by HIC, according to embodiments of the present disclosure.

FIG. 44 shows a graph indicating Construct 15 CH1-3/CT-tagged anti-nectin-4 conjugate was 99.5% monomeric as determined by analytical SEC, according to embodiments of the present disclosure.

FIG. 45 shows a graph indicating Construct 18 CH1-3/CT-tagged polatuzumab conjugate yielded a DAR of 6.57 as determined by PLRP, according to embodiments of the present disclosure.

FIG. 46 shows a graph indicating Construct 18 CH1-3/CT-tagged polatuzumab conjugate was 98.1% monomeric as determined by analytical SEC, according to embodiments of the present disclosure.

FIG. 47 shows a graph indicating Construct 18 CH1-3/CT-tagged anti-nectin-4 conjugate yielded a DAR of 6.1 as determined by PLRP, according to embodiments of the present disclosure.

FIG. 48 shows a graph indicating Construct 18 CH1-3/CT-tagged anti-nectin-4 conjugate was 96.6% monomeric as determined by analytical SEC, according to embodiments of the present disclosure.

FIG. 49 shows a graph indicating Construct 18 CH1-3/CT-tagged anti-FITC conjugate yielded a DAR of 6.16 as determined by PLRP, according to embodiments of the present disclosure.

FIG. 50 shows a graph indicating Construct 18 CH1-3/CT-tagged anti-FITC conjugate was 93.5% monomeric as determined by analytical SEC, according to embodiments of the present disclosure.

FIG. 51 shows a graph of in vitro cytotoxicity assays of free topoisomerase inhibitors in NCI-N87 gastric cancer cells.

FIG. 52 shows a graph of in vitro cytotoxicity assays of free topoisomerase inhibitors in Sk-Br-3 breast cancer cells.

FIG. 53 shows a graph of in vitro cytotoxicity assays of free topoisomerase inhibitors in Granta NHL cells.

FIG. 54 shows a graph of in vitro cytotoxicity assays of free topoisomerase inhibitors in MDA-MB-468 breast cancer cells.

FIG. 55 shows a graph of in vitro cytotoxicity assays of free topoisomerase inhibitors in MDA-PCa-2b prostate cancer cells.

FIG. 56 shows a graph of in vitro cytotoxicity assays in MDA-MB-468 breast cancer cells of a TROP-2 targeted ADC made using Compound 32, according to embodiments of the present disclosure.

FIG. 57 shows a graph of in vitro cytotoxicity assays in NCI-N87 gastric cancer cells of a HER2 targeted ADC made using Compound 32, according to embodiments of the present disclosure.

FIG. 58 shows a graph of in vitro cytotoxicity assays in SU-DHL-1 ALCL cells of a CD25 targeted ADC made using Compound 32, according to embodiments of the present disclosure.

FIG. 59 shows a graph of in vitro cytotoxicity assays in BxPC-3 pancreatic cancer cells of a TROP-2 targeted ADC made using Compound 32, according to embodiments of the present disclosure.

FIG. 60 shows a graph of in vitro cytotoxicity assays in NCI-N87 gastric cancer cells of a HER2 targeted ADC made using Compound 36, according to embodiments of the present disclosure.

FIG. 61 shows a graph of in vitro cytotoxicity assays in Sk-Br-3 breast cancer cells of a HER2 targeted ADC made using Compound 36, according to embodiments of the present disclosure.

FIG. 62 shows a schematic drawing of ELISA assays used to determine total antibody and ADC concentrations for pharmacokinetic (PK) sample analysis.

FIG. 63 shows a graph of concentration (μg/mL) vs. days post dose following a 0.9 mg/kg dose of trastuzumab antibody.

FIG. 64 shows a graph of concentration (μg/mL) vs. days post dose following a 0.9 mg/kg dose of polatuzumab antibody.

FIG. 65 shows a graph of concentration (μg/mL) vs. days post dose following a 0.9 mg/kg dose of a conventional HER2 topoisomerase inhibitor conjugated ADC bearing a protease-cleavable linker, according to embodiments of the present disclosure.

FIG. 66 shows a graph of concentration (μg/mL) vs. days post dose following a 0.9 mg/kg dose of CH1-3/CT-tagged trastuzumab conjugated to construct 32, according to embodiments of the present disclosure, according to embodiments of the present disclosure.

FIG. 67 shows a graph of concentration (μg/mL) vs. days post dose following a 0.9 mg/kg dose of CH1-3/CT-tagged trastuzumab conjugated to construct 36, according to embodiments of the present disclosure.

FIG. 68 shows a graph of concentration (μg/mL) vs. days post dose following a 0.9 mg/kg dose of CT-tagged polatuzumab conjugated to construct 15, according to embodiments of the present disclosure.

FIG. 69 shows a graph of concentration (μg/mL) vs. days post dose following a 0.9 mg/kg dose of CH2-3-tagged polatuzumab conjugated to construct 15, according to embodiments of the present disclosure.

FIG. 70 shows a graph of concentration (μg/mL) vs. days post dose following a 0.9 mg/kg dose of CH1-2-tagged polatuzumab conjugated to construct 15, according to embodiments of the present disclosure.

FIG. 71 shows a graph of mean tumor volume (mm³) vs. days, which indicates in vivo efficacy against an NCI-H292 xenograft of TROP-2 targeted ADCs carrying topoisomerase inhibitor payloads. n=8 mice/group; dosing is indicated by arrows, according to embodiments of the present disclosure.

FIG. 72 shows structures of dual-payload constructs carrying a MMAE payload and a second payload with an alternative mechanism of action (MOA), according to embodiments of the present disclosure.

FIG. 73 shows structures of dual-payload constructs carrying a belotecan payload and a second payload with an alternative MOA, according to embodiments of the present disclosure.

FIG. 74 shows a graph of mean tumor volume (mm³) vs. days, which indicates in vivo efficacy against an NCI-H292 xenograft of TROP-2 targeted ADCs carrying topoisomerase inhibitor payloads, according to embodiments of the present disclosure. n=7 mice/group. A single i.v. dose was delivered on Day 0.

FIG. 75 shows a graph of in vivo efficacy against an NCI-H₁₇₈₁ xenograft of nectin-4 targeted ADCs carrying topoisomerase inhibitor payloads, according to embodiments of the present disclosure. n=5 mice/group. A 5 mg/kg dose was delivered i.v. on Days 0 and 7.

FIG. 76 shows a graph of in vitro potency of HER2 targeted or isotype control ADCs carrying (36) or (175) as compared to free payload against NCI-N87 cells, according to embodiments of the present disclosure.

FIG. 77 shows a graph of in vitro potency of HER2 targeted or isotype control ADCs carrying (36) or (175) as compared to free payloads against SK-BR-3 cells, according to embodiments of the present disclosure.

FIG. 78 shows a graph of in vitro potency of TROP-2 targeted or isotype control ADCs carrying (36) or (80) as compared to (16) against MDA-MB-468 cells, according to embodiments of the present disclosure.

FIG. 79 shows a graph of in vitro potency of TROP-2 targeted or isotype control ADCs carrying (36) or (80) as compared to (16) against BxPC-3 cells, according to embodiments of the present disclosure.

FIG. 80 shows a graph of in vitro potency of HER2 targeted or isotype control ADCs carrying (99), (103), or (110) as compared to (16) against NCI-N87 cells, according to embodiments of the present disclosure.

FIG. 81 shows a graph of in vitro potency of HER2 targeted or isotype control ADCs carrying (36), (86), or (92) as compared to (16) against SK-BR-3 cells, according to embodiments of the present disclosure.

FIG. 82 shows a graph of in vitro potency of HER2 targeted or isotype control ADCs carrying (36), (86), or (92) as compared to (16) against NCI-N87 cells, according to embodiments of the present disclosure.

FIG. 83 shows a graph of in vitro potency of TROP-2 targeted or isotype control ADCs carrying (65), Trodelvy, or a CL2A-SN38 isotype control conjugate as compared to (1) or (2) against SK-BR-3 cells, according to embodiments of the present disclosure.

FIG. 84 shows a graph of in vitro potency of HER2 targeted or isotype control ADCs carrying (65), Enhertu, or a MC-GGFG-Dxd isotype control conjugate as compared to (2) against NCI-N87 cells, according to embodiments of the present disclosure.

FIG. 85 shows a graph of in vitro potency of TROP-2 targeted or isotype control ADCs carrying (36) or (113) as compared to (16) against MDA-MB-468 cells, according to embodiments of the present disclosure.

FIG. 86 shows a graph of in vitro potency of HER2 targeted or isotype control ADCs carrying (36), (136), or (142) as compared to (16) against NCI-N87 cells, according to embodiments of the present disclosure.

FIG. 87 shows a graph of in vitro potency of TROP-2 targeted or isotype control ADCs carrying (36), (127), or (131) as compared to (16) against MDA-MB-468 cells, according to embodiments of the present disclosure.

FIG. 88 shows a graph of in vitro potency of HER2 targeted or isotype control ADCs carrying (36), (127), or (131) as compared to (16) against NCI-N87 cells, according to embodiments of the present disclosure.

FIG. 89 shows a graph of in vitro potency of TROP-2 targeted or isotype control ADCs carrying (151), (147), or (131) as compared to (16) against NCI-N87 cells, according to embodiments of the present disclosure.

FIG. 90 shows a graph of in vitro potency of HER2 targeted or isotype control ADCs carrying (151), (147), or (131) as compared to (16) against SK-BR-3 cells, according to embodiments of the present disclosure.

FIG. 91 shows a graph indicating Compound 127 CH1-3/CT-tagged trastuzumab conjugate yielded a DAR of 7.15 as determined by PLRP.

FIG. 92 shows a graph indicating Compound 131 CH1-3/CT-tagged trastuzumab conjugate yielded a DAR of 6.80 as determined by PLRP.

FIG. 93 shows a graph indicating Compound 127 CH1-3/CT-tagged trastuzumab conjugate was 94.4% monomeric as determined by analytical SEC.

FIG. 94 shows a graph indicating Compound 131 CH1-3/CT-tagged trastuzumab conjugate was 93.6% monomeric as determined by analytical SEC.

FIG. 95 shows a graph indicating Compound 80 CH1-3/CT-tagged trastuzumab conjugate yielded a DAR of 5.86 as determined by PLRP.

FIG. 96 shows a graph indicating Compound 80 CH1-3/CT-tagged trastuzumab conjugate was 97.4% monomeric as determined by analytical SEC.

FIG. 97 shows a graph indicating Compound 80 CH1-3/CT-tagged sacituzumab conjugate yielded a DAR of 6.19 as determined by PLRP.

FIG. 98 shows a graph indicating Compound 80 CH1-3/CT-tagged sacituzumab conjugate was 97.1% monomeric as determined by analytical SEC.

FIG. 99 shows a graph indicating Compound 86 CH1-3/CT-tagged anti-FITC conjugate yielded a DAR of 5.46 as determined by PLRP.

FIG. 100 shows a graph indicating Compound 86 CH1-3/CT-tagged anti-FITC conjugate was 98.0% monomeric as determined by analytical SEC.

FIG. 101 shows a graph indicating Compound 92 CH1-3/CT-tagged anti-FITC conjugate yielded a DAR of 1.58 as determined by PLRP.

FIG. 102 shows a graph indicating Compound 92 CH1-3/CT-tagged anti-FITC conjugate was 96.1% monomeric as determined by analytical SEC.

FIG. 103 shows a graph indicating Compound 99 CH1-3/CT-tagged anti-FITC conjugate yielded a DAR of 3.07 as determined by PLRP.

FIG. 104 shows a graph indicating Compound 99 CH1-3/CT-tagged anti-FITC conjugate was 97.9% monomeric as determined by analytical SEC.

FIG. 105 shows a graph indicating Compound 103 CH1-3/CT-tagged trastuzumab conjugate yielded a DAR of 6.56 as determined by PLRP.

FIG. 106 shows a graph indicating Compound 103 CH1-3/CT-tagged trastuzumab conjugate was 97.3% monomeric as determined by analytical SEC.

FIG. 107 shows a graph indicating Compound 110 CH1-3/CT-tagged anti-FITC conjugate yielded a DAR of 5.66 as determined by PLRP.

FIG. 108 shows a graph indicating Compound 110 CH1-3/CT-tagged anti-FITC conjugate was 98.5% monomeric as determined by analytical SEC.

FIG. 109 shows a graph indicating Compound 113 CH1-3/CT-tagged sacituzumab conjugate yielded a DAR of 6.41 as determined by PLRP.

FIG. 110 shows a graph indicating Compound 113 CH1-3/CT-tagged sacituzumab conjugate was 97.4% monomeric as determined by analytical SEC.

FIG. 111 shows a graph indicating Compound 123 CH1-3/CT-tagged anti-FITC conjugate yielded a DAR of 5.56 as determined by PLRP.

FIG. 112 shows a graph indicating Compound 123 CH1-3/CT-tagged anti-FITC conjugate was 95.5% monomeric as determined by analytical SEC.

FIG. 113 shows a graph indicating Compound 123 CH1-3/CT-tagged sacituzumab conjugate yielded a DAR of 6.27 as determined by PLRP.

FIG. 114 shows a graph indicating Compound 123 CH1-3/CT-tagged sacituzumab conjugate was 98.0% monomeric as determined by analytical SEC.

FIG. 115 shows a graph indicating Compound 151 CH1-3/CT-tagged anti-FITC conjugate yielded a DAR of 5.67 as determined by PLRP.

FIG. 116 shows a graph indicating Compound 151 CH1-3/CT-tagged anti-FITC conjugate was 97.8% monomeric as determined by analytical SEC.

FIG. 117 shows a graph indicating Compound 147 CH1-3/CT-tagged anti-FITC conjugate yielded a DAR of 6.47 as determined by PLRP.

FIG. 118 shows a graph indicating Compound 147 CH1-3/CT-tagged anti-FITC conjugate was 96.4% monomeric as determined by analytical SEC.

FIG. 119 shows a graph indicating Compound 73 CH1-3/CT-tagged trastuzumab conjugate yielded a DAR of 5.41 as determined by PLRP.

FIG. 120 shows a graph indicating Compound 67 CH1-3/CT-tagged trastuzumab conjugate yielded a DAR of 4.02 as determined by PLRP.

FIG. 121 shows a graph indicating Compound 136 CH1-3/CT-tagged trastuzumab conjugate yielded a DAR of 7.26 as determined by PLRP.

FIG. 122 shows a graph indicating Compound 136 CH1-3/CT-tagged trastuzumab conjugate was 98.9% monomeric as determined by analytical SEC.

FIG. 123 shows a graph indicating Compound 142 CH1-3/CT-tagged trastuzumab conjugate yielded a DAR of 6.9 as determined by PLRP.

FIG. 124 shows a graph indicating Compound 175 CH1-3/CT-tagged anti-FITC conjugate yielded a DAR of 5.08 as determined by PLRP.

FIG. 125 shows a graph indicating Compound 175 CH1-3/CT-tagged anti-FITC conjugate was 93.0% monomeric as determined by analytical SEC.

FIG. 126 shows a graph indicating Compound 181 CT-tagged trastuzumab conjugate yielded a DAR of 3.45 as determined by HIC.

FIG. 127 shows a graph indicating Compound 181 CT-tagged trastuzumab conjugate was 97.6% monomeric as determined by analytical SEC.

FIG. 128 shows a graph indicating Compound 181 CT-tagged anti-MUC1 conjugate yielded a DAR of 3.59 as determined by HIC.

FIG. 129 shows a graph indicating Compound 181 CT-tagged anti-MUC1 conjugate was 99.0% monomeric as determined by analytical SEC.

FIG. 130 shows a graph indicating Compound 181 CH1/CT-tagged anti-MUC1 conjugate yielded a DAR of 2.13 as determined by PLRP.

FIG. 131 shows a graph indicating Compound 181 CH1/CT-tagged anti-MUC1 conjugate was 96.9% monomeric as determined by analytical SEC.

FIG. 132 shows a graph indicating Compound 181 CH1/CT-tagged trastuzumab conjugate yielded a DAR of 3.28 as determined by PLRP.

FIG. 133 shows a graph indicating Compound 181 CH1/CT-tagged trastuzumab conjugate was 92.2% monomeric as determined by analytical SEC.

FIG. 134 shows a graph indicating Compound 194 CT-tagged trastuzumab conjugate yielded a DAR of 3.04 as determined by HIC.

FIG. 135 shows a graph indicating Compound 194 CT-tagged trastuzumab conjugate was 97.2% monomeric as determined by analytical SEC.

FIG. 136 shows a graph indicating Compound 194 CT-tagged anti-MUC1 conjugate yielded a DAR of 3.01 as determined by HIC

FIG. 137 shows a graph indicating Compound 194 CT-tagged anti-MUC1 conjugate was 99.3% monomeric as determined by analytical SEC.

FIG. 138 shows a graph indicating Compound 194 CH1/CT-tagged anti-MUC1 conjugate yielded a DAR of 5.07 as determined by PLRP.

FIG. 139 shows a graph indicating Compound 194 CH1/CT-tagged anti-MUC1 conjugate was 97.0% monomeric as determined by analytical SEC.

FIG. 140 shows a graph indicating Compound 194 CH1/CT-tagged trastuzumab conjugate yielded a DAR of 6.77 as determined by PLRP.

FIG. 141 shows a graph indicating Compound 194 CH1/CT-tagged trastuzumab conjugate was 96.6% monomeric as determined by analytical SEC.

FIG. 142 shows a graph indicating Compound 200 CH1/CT-tagged trastuzumab conjugate yielded a DAR of 6.49 as determined by PLRP.

FIG. 143 shows a graph indicating Compound 200 CH1/CT-tagged trastuzumab conjugate was 94.2% monomeric as determined by analytical SEC.

FIG. 144 shows a graph indicating Compound 200 CH1/CT-tagged sacituzumab conjugate yielded a DAR of 6.19 as determined by PLRP.

FIG. 145 shows a graph indicating Compound 200 CH1/CT-tagged sacituzumab conjugate was 96.8% monomeric as determined by analytical SEC.

FIG. 146 shows a graph indicating Compound 200 CH1/CT-tagged anti-FITC conjugate yielded a DAR of 6.5 as determined by PLRP.

FIG. 147 shows a graph indicating Compound 200 CH1/CT-tagged anti-FITC conjugate is 94.4% monomeric as determined by analytical SEC.

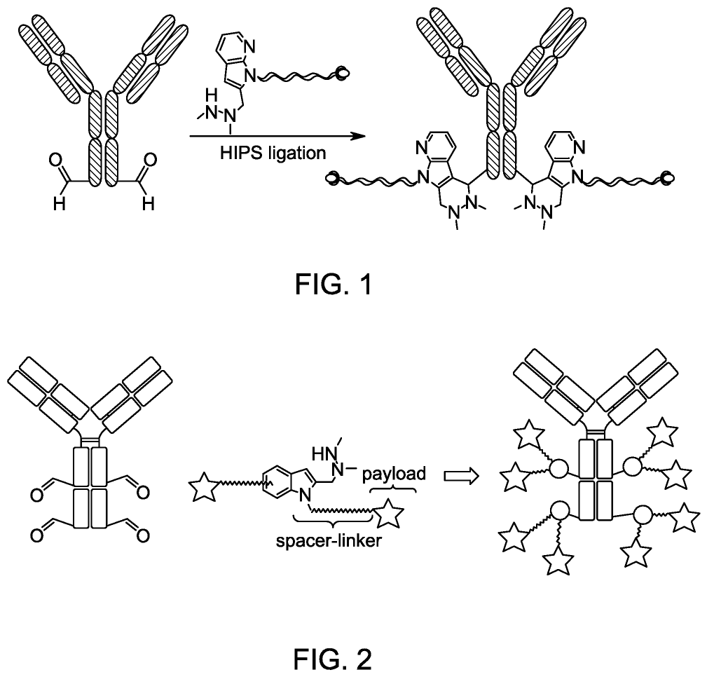

### DEFINITIONS

The following terms have the following meanings unless otherwise indicated. Any undefined terms have their art recognized meanings.

“Alkyl” refers to monovalent saturated aliphatic hydrocarbyl groups having from 1 to 10 carbon atoms and such as 1 to 6 carbon atoms, or 1 to 5, or 1 to 4, or 1 to 3 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain (except the C₁ carbon atom) have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR¹⁰—, —NR¹⁰C(O)—, —C(O)NR¹⁰— and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

The term “alkane” refers to alkyl group and alkylene group, as defined herein.

The term “alkylaminoalkyl”, “alkylaminoalkenyl” and “alkylaminoalkynyl” refers to the groups R′NHR″— where R′ is alkyl group as defined herein and R″ is alkylene, alkenylene or alkynylene group as defined herein.

The term “alkaryl” or “aralkyl” refers to the groups -alkylene-aryl and -substituted alkylene-aryl where alkylene, substituted alkylene and aryl are defined herein.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “alkoxyamino” refers to the group —NH-alkoxy, wherein alkoxy is defined herein.

The term “haloalkoxy” refers to the groups alkyl-O— wherein one or more hydrogen atoms on the alkyl group have been substituted with a halo group and include, by way of examples, groups such as trifluoromethoxy, and the like.

The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

The term “alkylthioalkoxy” refers to the group -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl and —SO₂-heteroaryl.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

The term “substituted alkynyl” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl, and —SO₂-heteroaryl.

“Alkynyloxy” refers to the group —O-alkynyl, wherein alkynyl is as defined herein. Alkynyloxy includes, by way of example, ethynyloxy, propynyloxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

“Acylamino” refers to the groups —NR²⁰C(O)alkyl, —NR²⁰C(O)substituted alkyl, N R²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²¹C(O)NR²²R²³ where R²¹, R²², and R²³ are independently selected from hydrogen, alkyl, aryl or cycloalkyl, or where two R groups are joined to form a heterocyclyl group.

The term “alkoxycarbonylamino” refers to the group —NRC(O)OR where each R is independently hydrogen, alkyl, substituted alkyl, aryl, heteroaryl, or heterocyclyl wherein alkyl, substituted alkyl, aryl, heteroaryl, and heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aminosulfonyl” refers to the group —SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R²¹ and R²² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group —N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O— alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O— substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkenyl” refers to non-aromatic cyclic alkyl groups of from 3 to 10 carbon atoms having single or multiple rings and having at least one double bond and preferably from 1 to 2 double bonds.

The term “substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —S0-heteroaryl, —SO₂-alkyl, —SO₂-substituted alkyl, —SO₂-aryl and —SO₂-heteroaryl.

“Cycloalkynyl” refers to non-aromatic cycloalkyl groups of from 5 to 10 carbon atoms having single or multiple rings and having at least one triple bond.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic. To satisfy valence requirements, any heteroatoms in such heteroaryl rings may or may not be bonded to H or a substituent group, e.g., an alkyl group or other substituent as described herein. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

The term “heteroaralkyl” refers to the groups -alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from nitrogen, sulfur, or oxygen, where, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties. To satisfy valence requirements, any heteroatoms in such heterocyclic rings may or may not be bonded to one or more H or one or more substituent group(s), e.g., an alkyl group or other substituent as described herein.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Heterocyclyloxy” refers to the group —O-heterocyclyl.

The term “heterocyclylthio” refers to the group heterocyclic-S—.

The term “heterocyclene” refers to the diradical group formed from a heterocycle, as defined herein.

The term “hydroxyamino” refers to the group —NHOH.

“Nitro” refers to the group —NO₂.

“Oxo” refers to the atom (═O).

“Sulfonyl” refers to the group —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cycloalkyl, —OSO₂-cycloalkenyl, —OSO₂-substituted cylcoalkenyl, —OSO₂-aryl, —OSO₂-substituted aryl, —OSO₂-heteroaryl, —OSO₂-substituted heteroaryl, —OSO₂-heterocyclic, and —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Sulfate” or “sulfate ester” refers the group —O—SO₂—OH, —O—SO₂—O-alkyl, —O—SO₂—O-substituted alkyl, —O—SO₂—O-alkenyl, —O—SO₂—O-substituted alkenyl, —O—SO₂—O-cycloalkyl, —O—SO₂—O-substituted cycloalkyl, —O—SO₂—O-cycloalkenyl, —O—SO₂—O-substituted cylcoalkenyl, —O—SO₂—O-aryl, —O—SO₂—O-substituted aryl, —O—SO₂—O-heteroaryl, —O—SO₂—O-substituted heteroaryl, —O—SO₂—O-heterocyclic, and —O—SO₂—O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

“Thiol” refers to the group —SH.

“Thioxo” or the term “thioketo” refers to the atom (═S).

“Alkylthio” or the term “thioalkoxy” refers to the group —S-alkyl, wherein alkyl is as defined herein. In certain embodiments, sulfur may be oxidized to —S(O)—. The sulfoxide may exist as one or more stereoisomers.

The term “substituted thioalkoxy” refers to the group —S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclyl-S— wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂ , O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

“Pharmaceutically effective amount” and “therapeutically effective amount” refer to an amount of a compound sufficient to treat a specified disorder or disease or one or more of its symptoms and/or to prevent the occurrence of the disease or disorder. In reference to tumorigenic proliferative disorders, a pharmaceutically or therapeutically effective amount comprises an amount sufficient to, among other things, cause the tumor to shrink or decrease the growth rate of the tumor.

“Patient” refers to human and non-human subjects, especially mammalian subjects.

The term “treating” or “treatment” as used herein means the treating or treatment of a disease or medical condition in a patient, such as a mammal (particularly a human) that includes: (a) preventing the disease or medical condition from occurring, such as, prophylactic treatment of a subject; (b) ameliorating the disease or medical condition, such as, eliminating or causing regression of the disease or medical condition in a patient; (c) suppressing the disease or medical condition, for example by, slowing or arresting the development of the disease or medical condition in a patient; or (d) alleviating a symptom of the disease or medical condition in a patient.

The terms “polypeptide,” “peptide,” and “protein” are used interchangeably herein to refer to a polymeric form of amino acids of any length. Unless specifically indicated otherwise, “polypeptide,” “peptide,” and “protein” can include genetically coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, proteins which contain at least one N-terminal methionine residue (e.g., to facilitate production in a recombinant host cell); immunologically tagged proteins; and the like. In certain embodiments, a polypeptide is an antibody.

“Native amino acid sequence” or “parent amino acid sequence” are used interchangeably herein to refer to the amino acid sequence of a polypeptide prior to modification to include at least one modified amino acid residue.

The terms “amino acid analog,” “unnatural amino acid,” and the like may be used interchangeably, and include amino acid-like compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or combinations thereof. For example, amino acid analogs may include α-hydroxy acids, and α-amino acids, and the like. Examples of amino acid analogs include, but are not limited to, sulfoalanine, and the like.

The terms “amino acid side chain” or “side chain of an amino acid” and the like may be used to refer to the substituent attached to the α-carbon of an amino acid residue, including natural amino acids, unnatural amino acids, and amino acid analogs. An amino acid side chain can also include an amino acid side chain as described in the context of the modified amino acids and/or conjugates described herein.

The term “carbohydrate” and the like may be used to refer to monomers units and/or polymers of monosaccharides, disaccharides, oligosaccharides, and polysaccharides. The term sugar may be used to refer to the smaller carbohydrates, such as monosaccharides, disaccharides. The term “carbohydrate derivative” includes compounds where one or more functional groups of a carbohydrate of interest are substituted (replaced by any convenient substituent), modified (converted to another group using any convenient chemistry) or absent (e.g., eliminated or replaced by H). A variety of carbohydrates and carbohydrate derivatives are available and may be adapted for use in the subject compounds and conjugates.

The term “glycoside” or “glycosyl” refers to a sugar molecule or group bound to a moiety via a glycosidic bond. For example, the moiety that the glycoside is bound to can be a cleavable linker as described herein. A glycosidic bond can link the glycoside to the other moiety through various types of bonds, such as, but not limited to, an O-glycosidic bond (an O-glycoside), an N-glycosidic bond (a glycosylamine), an S-glycosidic bond (a thioglycoside), or C-glycosidic bond (a C-glycoside or C-glycosyl). In some cases, glycosides can be cleaved from the moiety they are attached to, such as by chemically-mediated hydrolysis or enzymatically-mediated hydrolysis.

The term “antibody” is used in the broadest sense and includes monoclonal antibodies (including full length monoclonal antibodies), polyclonal antibodies, and multispecific antibodies (e.g., bispecific antibodies), humanized antibodies, single-chain antibodies, chimeric antibodies, antibody fragments (e.g., Fab fragments), and the like. An antibody is capable of binding a target antigen. (Janeway, C., Travers, P., Walport, M., Shlomchik (2001) Immuno Biology, 5th Ed., Garland Publishing, New York). A target antigen can have one or more binding sites, also called epitopes, recognized by complementarity determining regions (CDRs) formed by one or more variable regions of an antibody.

The term “natural antibody” refers to an antibody in which the heavy and light chains of the antibody have been made and paired by the immune system of a multi-cellular organism. Spleen, lymph nodes, bone marrow and serum are examples of tissues that produce natural antibodies. For example, the antibodies produced by the antibody producing cells isolated from a first animal immunized with an antigen are natural antibodies.

The term “humanized antibody” or “humanized immunoglobulin” refers to a non-human (e.g., mouse or rabbit) antibody containing one or more amino acids (in a framework region, a constant region or a CDR, for example) that have been substituted with a correspondingly positioned amino acid from a human antibody. In general, humanized antibodies produce a reduced immune response in a human host, as compared to a non-humanized version of the same antibody. Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332). In certain embodiments, framework substitutions are identified by modeling of the interactions of the CDR and framework residues to identify framework residues important for antigen binding and sequence comparison to identify unusual framework residues at particular positions (see, e.g., U.S. Pat. No. 5,585,089; Riechmann et al., Nature 332:323 (1988)). Additional methods for humanizing antibodies contemplated for use in the present invention are described in U.S. Pat. Nos. 5,750,078; 5,502,167; 5,705,154; 5,770,403; 5,698,417; 5,693,493; 5,558,864; 4,935,496; and 4,816,567, and PCT publications WO 98/45331 and WO 98/45332. In particular embodiments, a subject rabbit antibody may be humanized according to the methods set forth in US20040086979 and US20050033031. Accordingly, the antibodies described above may be humanized using methods that are well known in the art.

The term “chimeric antibodies” refer to antibodies whose light and heavy chain genes have been constructed, typically by genetic engineering, from antibody variable and constant region genes belonging to different species. For example, the variable segments of the genes from a mouse monoclonal antibody may be joined to human constant segments, such as gamma 1 and gamma 3. An example of a therapeutic chimeric antibody is a hybrid protein composed of the variable or antigen-binding domain from a mouse antibody and the constant or effector domain from a human antibody, although domains from other mammalian species may be used.

An immunoglobulin polypeptide immunoglobulin light or heavy chain variable region is composed of a framework region (FR) interrupted by three hypervariable regions, also called “complementarity determining regions” or “CDRs”. The extent of the framework region and CDRs have been defined (see, “Sequences of Proteins of Immunological Interest,” E. Kabat et al., U.S. Department of Health and Human Services, 1991). The framework region of an antibody, that is the combined framework regions of the constituent light and heavy chains, serves to position and align the CDRs. The CDRs are primarily responsible for binding to an epitope of an antigen.

A “parent Ig polypeptide” is a polypeptide comprising an amino acid sequence which lacks an aldehyde-tagged constant region as described herein. The parent polypeptide may comprise a native sequence constant region, or may comprise a constant region with pre-existing amino acid sequence modifications (such as additions, deletions and/or substitutions).

As used herein the term “isolated” is meant to describe a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound that is removed from its natural environment and is at least 60% free, at least 75% free, at least 80% free, at least 85% free, at least 90% free, at least 95% free, at least 98% free, or more than 98% free, from other components with which it is naturally associated.

The term “physiological conditions” is meant to encompass those conditions compatible with living cells, e.g., predominantly aqueous conditions of a temperature, pH, salinity, etc. that are compatible with living cells.

By “reactive partner” is meant a molecule or molecular moiety that specifically reacts with another reactive partner to produce a reaction product. Exemplary reactive partners include a cysteine or serine of a sulfatase motif and Formylglycine Generating Enzyme (FGE), which react to form a reaction product of a converted aldehyde tag containing a formylglycine (fGly) in lieu of cysteine or serine in the motif. Other exemplary reactive partners include an aldehyde of an fGly residue of a converted aldehyde tag (e.g., a reactive aldehyde group) and an “aldehyde-reactive reactive partner”, which comprises an aldehyde-reactive group and a moiety of interest, and which reacts to form a reaction product of a polypeptide having the moiety of interest conjugated to the polypeptide through the fGly residue.

“N-terminus” refers to the terminal amino acid residue of a polypeptide having a free amine group, which amine group in non-N-terminus amino acid residues normally forms part of the covalent backbone of the polypeptide.

“C-terminus” refers to the terminal amino acid residue of a polypeptide having a free carboxyl group, which carboxyl group in non-C-terminus amino acid residues normally forms part of the covalent backbone of the polypeptide.

By “internal site” as used in referenced to a polypeptide or an amino acid sequence of a polypeptide means a region of the polypeptide that is not at the N-terminus or at the C-terminus.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also specifically embraced by the present invention and are disclosed herein just as if each and every such sub-combination was individually and explicitly disclosed herein.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides antibody-drug conjugate structures, that include a branched HIPS linker. The disclosure also encompasses compounds and methods for production of such conjugates, as well as methods of using the same.

### Antibody-Drug Conjugates

The present disclosure provides a conjugate, e.g., an antibody-drug conjugate (ADC). By “conjugate” is meant a polypeptide (e.g., an antibody) covalently attached to two or more other moieties (e.g., a drugs or active agents). For example, an antibody-drug conjugate according to the present disclosure includes two or more drugs or active agents covalently attached to an antibody. In certain embodiments, the polypeptide (e.g., antibody) and the two or more drugs or active agents are bound to each other through one or more functional groups and covalent bonds. For example, the one or more functional groups and covalent bonds can include a branched linker as described herein.

In certain embodiments, the conjugate is a polypeptide conjugate, which includes a polypeptide (e.g., an antibody) conjugated to two or more other moieties. In certain embodiments, the two or more moieties conjugated to the polypeptide can each independently be any of a variety of moieties of interest such as, but not limited to, a drug, an active agent, a detectable label, a water-soluble polymer, or a moiety for immobilization of the polypeptide to a membrane or a surface. In certain embodiments, the conjugate is a drug conjugate, where a polypeptide is an antibody, thus providing an antibody-drug conjugate (ADC). For instance, the conjugate can be a drug conjugate, where a polypeptide is conjugated to two or more drugs or active agents. Various types of drugs or active agents may be used in the conjugates and are described in more detail below.

Moieties of interest (e.g., drugs or active agents) can be conjugated to the polypeptide (e.g., antibody) at any desired site of the polypeptide. Thus, the present disclosure provides, for example, a polypeptide having moieties conjugated at two or more sites on the polypeptide, such as a site at or near the C-terminus of the polypeptide, a position at or near the N-terminus of the polypeptide, and a position between the C-terminus and the N-terminus of the polypeptide (e.g., at an internal site of the polypeptide). Combinations of the above conjugation sites are also possible.

In certain embodiments, a conjugate of the present disclosure includes two (or more) drugs or active agents conjugated to an amino acid residue of a polypeptide at the α-carbon of an amino acid residue. Stated another way, a conjugate includes a polypeptide where the side chain of an amino acid residue in the polypeptide has been modified and attached to two (or more) drugs or active agents (e.g., attached to two drugs or active agents through a branched linker as described herein). For example, a conjugate includes a polypeptide where the α-carbon of an amino acid residue in the polypeptide has been modified and attached to two drugs or active agents (e.g., attached to two drugs or active agents through a branched linker as described herein).

Embodiments of the present disclosure include conjugates where a polypeptide is conjugated to two or more moieties, such as 3 moieties, 4 moieties, 5 moieties, 6 moieties, 7 moieties, 8 moieties, 9 moieties, 10 moieties, 11 moieties, 12 moieties, 13 moieties, 14 moieties, 15 moieties, 16 moieties, 17 moieties, 18 moieties, 19 moieties, or 20 or more moieties. The moieties may be conjugated to the polypeptide at multiple sites in the polypeptide. In some embodiments, two moieties may be conjugated to a single amino acid residue of the polypeptide. For instance, two moieties may be conjugated to the same amino acid residue of the polypeptide. In other embodiments, two moieties are conjugated to a first amino acid residue of the polypeptide and two other moieties are conjugated to a second amino acid residue of the polypeptide. For example, a polypeptide can be conjugated to first and second moieties at a first amino acid residue and conjugated to third and fourth moieties at a second amino acid residue, etc. In some cases, two or more amino acid residues in the polypeptide are each conjugated to a pair of moieties (i.e., two moieties), where each pair of moieties is conjugated to the polypeptide through a branched linker as described herein. In some cases, 1 amino acid residue in the polypeptide is conjugated to a pair of moieties through a branched linker as described herein. In other instances, 2 or more amino acid residues, such as 3, 4, 5, 6, 7, 8, 9, or 10 or more amino acid residues in the polypeptide are each conjugated to a pair of moieties through a branched linker as described herein.

The one or more amino acid residues of the polypeptide that are conjugated to the moieties of interest may be naturally occurring amino acids, unnatural amino acids, or combinations thereof. For instance, the conjugate may include moieties of interest (e.g., drugs or active agents) conjugated to a naturally occurring amino acid residue of the polypeptide. In other instances, the conjugate may include moieties of interest conjugated to an unnatural amino acid residue of the polypeptide. The moieties of interest may be conjugated to the polypeptide at a single natural or unnatural amino acid residue as described above. One or more natural or unnatural amino acid residues in the polypeptide may be conjugated to the moieties of interest as described herein. For example, two (or more) amino acid residues (e.g., natural or unnatural amino acid residues) in the polypeptide may each be conjugated to two moieties through a branched linker, such that multiple sites in the polypeptide are conjugated to the moieties of interest.

As described herein, a polypeptide may be conjugated to two or more moieties of interest. In certain embodiments, the moiety of interest is a payload, for instance, a chemical entity, such as a drug, an active agent, or a detectable label. For example, drugs (or active agents) may be conjugated to the polypeptide, or in other embodiments, detectable labels may be conjugated to the polypeptide. In other embodiments, combinations of different payloads may be conjugated to the poypeptide. Thus, for instance, embodiments of the present disclosure include, but are not limited to, the following: a conjugate of a polypeptide and two or more drugs; a conjugate of a polypeptide and two or more active agents; a conjugate of a polypeptide and two or more detectable labels; and combinations thereof.

In certain embodiments, the polypeptide (e.g., antibody) and the moieties of interest (e.g., drugs or active agents) are conjugated through a conjugation moiety. For example, the polypeptide and the moieties of interest may each be bound (e.g., covalently bonded) to the conjugation moiety, thus indirectly binding the polypeptide and the moieties of interest together through the conjugation moiety. In some cases, the conjugation moiety includes a hydrazinyl-indolyl or a hydrazinyl-pyrrolo-pyridinyl compound, or a derivative of a hydrazinyl-indolyl or a hydrazinyl-pyrrolo-pyridinyl compound. For instance, a general scheme for coupling moieties of interest to a polypeptide through a hydrazinyl-indolyl or a hydrazinyl-pyrrolo-pyridinyl conjugation moiety is shown in the general reaction scheme below. Hydrazinyl-indolyl and hydrazinyl-pyrrolo-pyridinyl conjugation moieties are also referred to herein as a hydrazino-iso-Pictet-Spengler (HIPS) conjugation moiety and an aza-hydrazino-iso-Pictet-Spengler (azaHIPS) conjugation moiety, respectively.

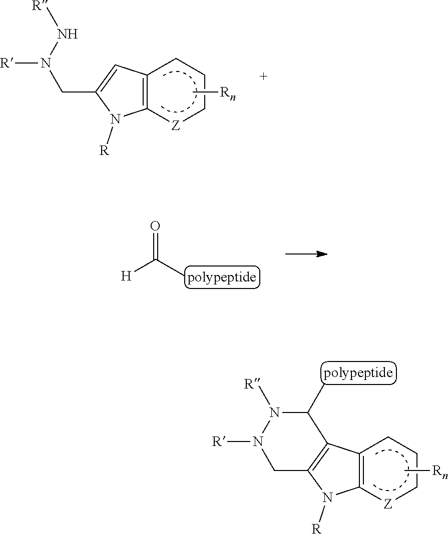

In the reaction scheme above, each R independently includes a moiety of interest (e.g., drug or active agent) that is conjugated to the polypeptide (e.g., conjugated to the polypeptide through a linker as described herein), where n is an integer from 1 to 4. As shown in the reaction scheme above, a conjugation moiety (e.g., a hydrazinyl-indolyl or a hydrazinyl-pyrrolo-pyridinyl conjugation moiety) is attached to two or more drugs or active agents, R. A polypeptide that includes a 2-formylglycine residue (fGly) is reacted with the conjugation moiety to produce a polypeptide conjugate, thus attaching the two or more drugs or active agents to the polypeptide through the conjugation moiety.

As described herein, the moieties can be any of a variety of moieties such as, but not limited to, chemical entities, such as detectable labels, or a drugs or active agents. R′ and R″ may each independently be any desired substituent, such as, but not limited to, hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. Z may be CR²¹, NR²², N, O or S, where R²¹ and R²² are each independently selected from any of the substituents described for R′ and R″ above.

Other hydrazinyl-indolyl or hydrazinyl-pyrrolo-pyridinyl conjugation moieties are also possible, as shown in the conjugates and compounds described herein. For example, the hydrazinyl-indolyl or hydrazinyl-pyrrolo-pyridinyl conjugation moieties may be attached (e.g., covalently attached) to two or more linkers. As such, embodiments of the present disclosure include a hydrazinyl-indolyl or hydrazinyl-pyrrolo-pyridinyl conjugation moiety attached to two or more drugs or active agents each through a corresponding linker. Thus, conjugates of the present disclosure may include two or more linkers, where each linker attaches a corresponding drug or active agent to the hydrazinyl-indolyl or hydrazinyl-pyrrolo-pyridinyl conjugation moiety. Accordingly, the hydrazinyl-indolyl or hydrazinyl-pyrrolo-pyridinyl conjugation moiety and two or more linkers may be viewed overall as a “branched linker”, where the hydrazinyl-indolyl or hydrazinyl-pyrrolo-pyridinyl conjugation moiety is attached to two of more “branches”, where each branch includes a linker attached to a drug or active agent.

Combinations of the same of different payloads may be conjugated to the poypeptide through the branched linker. In certain embodiments, the two payloads (e.g., drugs, active agents or detectable labels) attached to the branched linker are the same payload (e.g., drug, active agent or detectable label). For example, a first branch of a branched linker may be attached to a payload (e.g., drug, active agent or detectable label) and a second branch of the branched linker may be attached to the same payload (e.g., drug, active agent or detectable label) as the first branch.

In other embodiments, the two payloads (e.g., drugs, active agents or detectable labels) attached to the branched linker are different payloads (e.g., drugs, active agents or detectable labels). For example, a first branch of a branched linker may be attached to a first payload (e.g., a first drug, active agent or detectable label) and a second branch of the branched linker may be attached to a second payload (e.g., a second drug, active agent or detectable label) different from the first payload (e.g., the first drug, active agent or detectable label) attached to the first branch.

In some embodiments, where two different drugs or active agents are attached to the branched linker, the drugs or active agents may be selected from drugs and active agents that have a synergistic therapeutic effect. By “synergistic”, “synergism” or “synergy” is meant a therapeutic effect that is greater than the sum of the effects of the drugs or active agents taken separately. For example, in some instances, the use of two different drugs or active agents attached to the branched linker may provide a lower therapeutically effective concentration at which both payloads act, thereby increasing overall potency of the ADC.

In some embodiments, where two different drugs or active agents are attached to the branched linker, the drugs or active agents may be selected from drugs and active agents that provide an enhanced therapeutic benefit as compared to the use of the drugs or active agents separately, For example, the drugs or active agents may provide an increased effect on drug delivery of the ADC (e.g., some payloads, such as the iRGD peptide, can increase extravasation into tissues and augment tumor penetration).

In some embodiments, where two different drugs or active agents are attached to the branched linker, the drugs or active agents may be selected from drugs and active agents that use different mechanisms of action. In some cases, this may provide a decrease in tumor drug resistance by targeting multiple pathways. Examples of payload combinations can include, but are not limited to, cytotoxic drugs, immunomodulatory molecules to activate or inhibit immune cell populations, cytokines, hormones, chelating agents loaded with radioisotopes, and the like.

In some embodiments, where two different payloads are attached to the branched linker, the payloads may be selected from combinations of drugs or active agents and detectable labels. For example, a first payload may be a detectable labels that is used as an imaging agent or tracer to detect the location of the ADC in vivo, while a second payload may be a drug or active agent that provides a therapeutic activity.

Various embodiments of the linkers that may couple the hydrazinyl-indolyl or hydrazinyl-pyrrolo-pyridinyl conjugation moiety to the drugs or active agents are described in detail herein. For example, in some instances, the linker is a cleavable linker, such as a cleavable linker as described herein.

In certain embodiments, the polypeptide may be conjugated to two or more moieties of interest, where one or more amino acids of the polypeptide are modified before conjugation to the moieties of interest. Modification of one or more amino acids of the polypeptide may produce a polypeptide that contains one or more reactive groups suitable for conjugation to the moieties of interest. In some cases, the polypeptide may include one or more modified amino acid residues to provide one or more reactive groups suitable for conjugation to the moieties of interest (e.g., where two or more moieties are attached to a conjugation moiety, such as a hydrazinyl-indolyl or a hydrazinyl-pyrrolo-pyridinyl conjugation moiety as described above). For example, an amino acid of the polypeptide may be modified to include a reactive aldehyde group (e.g., a reactive aldehyde). A reactive aldehyde may be included in an “aldehyde tag” or “ald-tag”, which as used herein refers to an amino acid sequence derived from a sulfatase motif (e.g., L(C/S)TPSR) that has been converted by action of a formylglycine generating enzyme (FGE) to contain a 2-formylglycine residue (referred to herein as “fGly”). The fGly residue generated by an FGE may also be referred to as a “formylglycine”. Stated differently, the term “aldehyde tag” is used herein to refer to an amino acid sequence that includes a “converted” sulfatase motif (i.e., a sulfatase motif in which a cysteine or serine residue has been converted to fGly by action of an FGE, e.g., L(fGly)TPSR). A converted sulfatase motif may be produced from an amino acid sequence that includes an “unconverted” sulfatase motif (i.e., a sulfatase motif in which the cysteine or serine residue has not been converted to fGly by an FGE, but is capable of being converted, e.g., an unconverted sulfatase motif with the sequence: L(C/S)TPSR). By “conversion” as used in the context of action of a formylglycine generating enzyme (FGE) on a sulfatase motif refers to biochemical modification of a cysteine or serine residue in a sulfatase motif to a formylglycine (fGly) residue (e.g., Cys to fGly, or Ser to fGly). Additional aspects of aldehyde tags and uses thereof in site-specific protein modification are described in U.S. Pat. Nos. 7,985,783 and 8,729,232, the disclosures of each of which are incorporated herein by reference.

In some cases, to produce the conjugate, the polypeptide containing the fGly residue may be conjugated to the moieties of interest by reaction of the fGly with a compound (e.g., a compound containing a hydrazinyl-indolyl or a hydrazinyl-pyrrolo-pyridinyl conjugation moiety, as described above). For example, an fGly-containing polypeptide may be contacted with a reactive partner under conditions suitable to provide for conjugation of two or more drugs to the polypeptide. In some instances, the reactive partner may include a hydrazinyl-indolyl or a hydrazinyl-pyrrolo-pyridinyl conjugation moiety as described above. For example, two or more drugs or active agents may be attached to a hydrazinyl-indolyl or a hydrazinyl-pyrrolo-pyridinyl conjugation moiety. In some cases, the drugs or active agents are attached to a hydrazinyl-indolyl or a hydrazinyl-pyrrolo-pyridinyl conjugation moiety, such as covalently attached to a hydrazinyl-indolyl or a hydrazinyl-pyrrolo-pyridinyl, where each drug or active agent is attached through a corresponding linker to the hydrazinyl-indolyl or a hydrazinyl-pyrrolo-pyridinyl conjugation moiety.

In certain embodiments, a conjugate of the present disclosure includes a polypeptide (e.g., an antibody) having at least one amino acid residue that has been attached to two or more moieties of interest (e.g., drugs or active agents). In order to make the conjugate, an amino acid residue of the polypeptide may be modified and then coupled to two or more drugs or active agents attached to a hydrazinyl-indolyl or a hydrazinyl-pyrrolo-pyridinyl conjugation moiety as described above. In certain embodiments, an amino acid residue of the polypeptide (e.g., antibody) is a cysteine or serine residue that is modified to an fGly residue, as described above. In certain embodiments, the modified amino acid residue (e.g., fGly residue) is conjugated to two or more drugs or active agents containing a hydrazinyl-indolyl or a hydrazinyl-pyrrolo-pyridinyl conjugation moiety as described above to provide a conjugate of the present disclosure where the two or more drugs or active agents are conjugated to the polypeptide through the hydrazinyl-indolyl or hydrazinyl-pyrrolo-pyridinyl conjugation moiety. As used herein, the term fGly′ refers to the amino acid residue of the polypeptide (e.g., antibody) that is coupled to the moieties of interest (e.g., drugs or active agents).

In certain embodiments, the conjugate includes a polypeptide (e.g., an antibody) having at least one amino acid residue attached to a branched linker as described herein, which in turn is attached to two or more drugs or active agents. For instance, the conjugate may include a polypeptide (e.g., an antibody) having at least one amino acid residue (fGly′) that is conjugated to the moieties of interest (e.g., drugs or active agents) as described above.

Aspects of the present disclosure include a conjugate of formula (I):

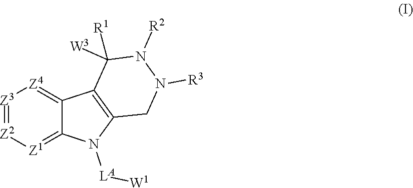

wherein:
   Z¹, Z², Z³ and Z⁴ are each independently selected from CR⁴, N and C-L^{B}-W², wherein at least one Z¹, Z², Z³ and Z⁴ is C-L^{B}-W²; R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl; R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
   each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
   L^{A} is a first linker;
   L^{B} is a second linker;
   W¹ is a first drug;
   W² is a second drug; and
   W³ is a polypeptide.

The substituents related to conjugates of formula (I) are described in more detail below.

In certain embodiments, Z¹, Z², Z³ and Z⁴ are each independently selected from CR⁴, N and C-L^{B}-W², wherein at least one Z¹, Z², Z³ and Z⁴ is C-L^{B}-W². In certain embodiments, Z¹ is CR⁴. In certain embodiments, Z¹ is N. In certain embodiments, Z¹ is C-L^{B}-W². In certain embodiments, Z² is CR⁴. In certain embodiments, Z² is N. In certain embodiments, Z² is C-L^{B}-W². In certain embodiments, Z³ is CR⁴. In certain embodiments, Z³ is N. In certain embodiments, Z³ is C-L^{B}-W². In certain embodiments, Z⁴ is CR⁴. In certain embodiments, Z⁴ is N. In certain embodiments, Z⁴ is C-L^{B}-W².

Combinations of various Z¹, Z², Z³ and Z⁴ are possible. For example, in some instances, Z¹ is C-L^{B}-W², Z² is CR⁴, Z³ is CR⁴, and Z⁴ is CR⁴. In some instances, Z¹ is CR⁴, Z² is C-L^{B}-W², Z³ is CR⁴, and Z⁴ is CR⁴. In some instances, Z¹ is CR⁴, Z² is CR⁴, Z³ is C-L^{B}-W², and Z⁴ is CR⁴. In some instances, Z¹ is CR⁴, Z² is CR⁴, Z³ is CR⁴, and Z⁴ is C-L^{B}-W².

In certain embodiments, R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, substituted heterocyclyl. In certain embodiments, R¹ is hydrogen. In certain embodiments, R¹ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹ is alkynyl or substituted alkynyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl.

In certain embodiments, R² is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R² is hydrogen. In certain embodiments, R² is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R² is methyl. In certain embodiments, R² is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R² is alkynyl or substituted alkynyl. In certain embodiments, R² is alkoxy or substituted alkoxy. In certain embodiments, R² is amino or substituted amino. In certain embodiments, R² is carboxyl or carboxyl ester. In certain embodiments, R² is acyl or acyloxy. In certain embodiments, R² is acyl amino or amino acyl. In certain embodiments, R² is alkylamide or substituted alkylamide. In certain embodiments, R² is sulfonyl. In certain embodiments, R² is thioalkoxy or substituted thioalkoxy. In certain embodiments, R² is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R² is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R² is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R² is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³ is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R³ is hydrogen. In certain embodiments, R³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³ is methyl. In certain embodiments, R³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³ is alkynyl or substituted alkynyl. In certain embodiments, R³ is alkoxy or substituted alkoxy. In certain embodiments, R³ is amino or substituted amino. In certain embodiments, R³ is carboxyl or carboxyl ester. In certain embodiments, R³ is acyl or acyloxy. In certain embodiments, R³ is acyl amino or amino acyl. In certain embodiments, R³ is alkylamide or substituted alkylamide. In certain embodiments, R³ is sulfonyl. In certain embodiments, R³ is thioalkoxy or substituted thioalkoxy. In certain embodiments, R³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiment, both R² and R³ are methyl.

In certain embodiments, R² and R³ are optionally cyclically linked to form a 5 or 6-membered heterocyclyl. In certain embodiments, R² and R³ are cyclically linked to form a 5 or 6-membered heterocyclyl. In certain embodiments, R² and R³ are cyclically linked to form a 5-membered heterocyclyl. In certain embodiments, R² and R³ are cyclically linked to form a 6-membered heterocyclyl.

In certain embodiments, each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

The various possibilities for each R⁴ are described in more detail as follows. In certain embodiments, R⁴ is hydrogen. In certain embodiments, each R⁴ is hydrogen. In certain embodiments, R⁴ is halogen, such as F, Cl, Br or I. In certain embodiments, R⁴ is F. In certain embodiments, R⁴ is Cl. In certain embodiments, R⁴ is Br. In certain embodiments, R⁴ is I. In certain embodiments, R⁴ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R⁴ is methyl. In certain embodiments, R⁴ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R⁴ is alkynyl or substituted alkynyl. In certain embodiments, R⁴ is alkoxy or substituted alkoxy. In certain embodiments, R⁴ is amino or substituted amino. In certain embodiments, R⁴ is carboxyl or carboxyl ester. In certain embodiments, R⁴ is acyl or acyloxy. In certain embodiments, R⁴ is acyl amino or amino acyl. In certain embodiments, R⁴ is alkylamide or substituted alkylamide. In certain embodiments, R⁴ is sulfonyl. In certain embodiments, R⁴ is thioalkoxy or substituted thioalkoxy. In certain embodiments, R⁴ is aryl or substituted aryl, such as C₅₋₈ aryl or C₅₋₈ substituted aryl, such as a C₅ aryl or C₅ substituted aryl, or a C₆ aryl or C₆ substituted aryl (e.g., phenyl or substituted phenyl). In certain embodiments, R⁴ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R⁴ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R⁴ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, L^{A} is a first linker. Examples of linkers that can be used in the conjugates of the present disclosure are described in more detail below.

In certain embodiments, L^{B} is a second linker. Examples of linkers that can be used in the conjugates of the present disclosure are described in more detail below.

In certain embodiments, W¹ is a first drug (or a first active agent). Examples of drugs and active agents that can be used in the conjugates of the present disclosure are described in more detail below.

In certain embodiments, W² is a second drug (or a second active agent). Examples of drugs and active agents that can be used in the conjugates of the present disclosure are described in more detail below.

In certain embodiments, W³ is a polypeptide (e.g., an antibody). In certain embodiments, W³ comprises one or more fGly′ residues as described herein. In certain embodiments, the polypeptide is attached to the rest of the conjugate through an fGly′ residue as described herein. Examples of polypeptides and antibodies that can be used in the conjugates of the present disclosure are described in more detail below.

In certain embodiments, the conjugate of formula (I) includes a first linker, L^{A}. The first linker, L^{A}, may be utilized to bind a first moiety of interest (e.g., a first drug or active agent) to a polypeptide (e.g., an antibody) through a conjugation moiety. The first linker, L^{A}, may be bound (e.g., covalently bonded) to the conjugation moiety (e.g., as described herein). For example, the first linker, L^{A}, may attach a hydrazinyl-indolyl or a hydrazinyl-pyrrolo-pyridinyl conjugation moiety to a first drug. The hydrazinyl-indolyl or hydrazinyl-pyrrolo-pyridinyl conjugation moiety may be used to conjugate the first linker, L^{A}, (and thus the first drug) to a polypeptide, such as an antibody.

For example, as shown in formula (I) above, L^{A} is attached to W³ through a conjugation moiety, and thus W³ is indirectly bonded to the linker L^{A} through the hydrazinyl-indolyl or a hydrazinyl-pyrrolo-pyridinyl conjugation moiety. As described above, W³ is a polypeptide (e.g., an antibody), and thus L^{A} is attached through the hydrazinyl-indolyl or a hydrazinyl-pyrrolo-pyridinyl conjugation moiety to the polypeptide (antibody), e.g., the linker L^{A} is indirectly bonded to the polypeptide (antibody) through the hydrazinyl-indolyl or a hydrazinyl-pyrrolo-pyridinyl conjugation moiety.

Any convenient linker may be utilized for the first linker L^{A} in the subject conjugates and compounds. In certain embodiments, the first linker L^{A} may include a group selected from alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl. In certain embodiments, the first linker L^{A} may include an alkyl or substituted alkyl group. In certain embodiments, the first linker L^{A} may include an alkenyl or substituted alkenyl group. In certain embodiments, the first linker L^{A} may include an alkynyl or substituted alkynyl group. In certain embodiments, the first linker L^{A} may include an alkoxy or substituted alkoxy group. In certain embodiments, the first linker L^{A} may include an amino or substituted amino group. In certain embodiments, the first linker L^{A} may include a carboxyl or carboxyl ester group. In certain embodiments, the first linker L^{A} may include an acyl amino group. In certain embodiments, the first linker L^{A} may include an alkylamide or substituted alkylamide group. In certain embodiments, the first linker L^{A} may include an aryl or substituted aryl group. In certain embodiments, the first linker L^{A} may include a heteroaryl or substituted heteroaryl group. In certain embodiments, the first linker L^{A} may include a cycloalkyl or substituted cycloalkyl group. In certain embodiments, the first linker L^{A} may include a heterocyclyl or substituted heterocyclyl group.

In certain embodiments, the first linker L^{A} may include a polymer. For example, the polymer may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In certain embodiments, the polymer is a polyalkylene glycol. In certain embodiments, the polymer is a polyethylene glycol. Other linkers are also possible, as shown in the conjugates and compounds described in more detail below.

In some embodiments, L^{A} is a first linker described by the formula:

-(L¹)ₐ-(L²)_{b}-(L³)_{c}-(L⁴)ₐ-(L⁵)ₑ-(L⁶)_{f}-,
wherein L¹, L², L³, L⁴, L⁵ and L⁶ are each independently a linker subunit, and a, b, c, d, e and f are each independently 0 or 1.

In certain embodiments, the sum of a, b, c, d, e and f is 0 to 6. In certain embodiments, the sum of a, b, c, d, e and f is 0. In certain embodiments, the sum of a, b, c, d, e and f is 1. In certain embodiments, the sum of a, b, c, d, e and f is 2. In certain embodiments, the sum of a, b, c, d, e and f is 3. In certain embodiments, the sum of a, b, c, d, e and f is 4. In certain embodiments, the sum of a, b, c, d, e and f is 5. In certain embodiments, the sum of a, b, c, d, e and f is 6. In certain embodiments, a, b, c, d, e and f are each 1. In certain embodiments, a, b, c, d and e are each 1 and f is 0. In certain embodiments, a, b, c and d are each 1 and e and f are each 0. In certain embodiments, a, b, and c are each 1 and d, e and f are each 0. In certain embodiments, a and b are each 1 and c, d, e and f are each 0. In certain embodiments, a is 1 and b, c, d, e and f are each 0.

In certain embodiments, the linker subunit L¹ is attached to the hydrazinyl-indolyl or a hydrazinyl-pyrrolo-pyridinyl conjugation moiety (e.g., as shown in formula (I) above). In certain embodiments, the linker subunit L², if present, is attached to the first drug or active agent W¹. In certain embodiments, the linker subunit L³, if present, is attached to the first drug or active agent W¹. In certain embodiments, the linker subunit L⁴, if present, is attached to the first drug or active agent W¹. In certain embodiments, the linker subunit L⁵, if present, is attached to the first drug or active agent W¹. In certain embodiments, the linker subunit L⁶, if present, is attached to the first drug or active agent W¹.

Any convenient linker subunits may be utilized in the first linker L^{A}. Linker subunits of interest include, but are not limited to, units of polymers such as polyethylene glycols, polyethylenes and polyacrylates, amino acid residue(s), carbohydrate-based polymers or carbohydrate residues and derivatives thereof, polynucleotides, alkyl groups, aryl groups, heterocyclic groups, combinations thereof, and substituted versions thereof. In some embodiments, each of L¹, L², L³, L⁴, L⁵ and L⁶ (if present) comprise one or more groups independently selected from a polyethylene glycol, a modified polyethylene glycol, an amino acid residue, an alkyl group, a substituted alkyl, an aryl group, a substituted aryl group, and a diamine (e.g., a linking group that includes an alkylene diamine).

In some embodiments, L¹ (if present) comprises a polyethylene glycol, a modified polyethylene glycol, an amino acid residue, an alkyl group, a substituted alkyl, an aryl group, a substituted aryl group, or a diamine. In some embodiments, L¹ comprises a polyethylene glycol. In some embodiments, L¹ comprises a modified polyethylene glycol. In some embodiments, L₁ comprises an amino acid residue. In some embodiments, L¹ comprises an alkyl group or a substituted alkyl. In some embodiments, L¹ comprises an aryl group or a substituted aryl group. In some embodiments, L¹ comprises a diamine (e.g., a linking group comprising an alkylene diamine).

In some embodiments, L² (if present) comprises a polyethylene glycol, a modified polyethylene glycol, an amino acid residue, an alkyl group, a substituted alkyl, an aryl group, a substituted aryl group, or a diamine. In some embodiments, L² comprises a polyethylene glycol. In some embodiments, L² comprises a modified polyethylene glycol. In some embodiments, L² comprises an amino acid residue. In some embodiments, L² comprises an alkyl group or a substituted alkyl. In some embodiments, L² comprises an aryl group or a substituted aryl group. In some embodiments, L² comprises a diamine (e.g., a linking group comprising an alkylene diamine).

In some embodiments, L³ (if present) comprises a polyethylene glycol, a modified polyethylene glycol, an amino acid residue, an alkyl group, a substituted alkyl, an aryl group, a substituted aryl group, or a diamine. In some embodiments, L³ comprises a polyethylene glycol. In some embodiments, L³ comprises a modified polyethylene glycol. In some embodiments, L³ comprises an amino acid residue. In some embodiments, L³ comprises an alkyl group or a substituted alkyl. In some embodiments, L³ comprises an aryl group or a substituted aryl group. In some embodiments, L³ comprises a diamine (e.g., a linking group comprising an alkylene diamine).

In some embodiments, L⁴ (if present) comprises a polyethylene glycol, a modified polyethylene glycol, an amino acid residue, an alkyl group, a substituted alkyl, an aryl group, a substituted aryl group, or a diamine. In some embodiments, L⁴ comprises a polyethylene glycol. In some embodiments, L⁴ comprises a modified polyethylene glycol. In some embodiments, L⁴ comprises an amino acid residue. In some embodiments, L⁴ comprises an alkyl group or a substituted alkyl. In some embodiments, L⁴ comprises an aryl group or a substituted aryl group. In some embodiments, L⁴ comprises a diamine (e.g., a linking group comprising an alkylene diamine).

In some embodiments, L⁵ (if present) comprises a polyethylene glycol, a modified polyethylene glycol, an amino acid residue, an alkyl group, a substituted alkyl, an aryl group, a substituted aryl group, or a diamine. In some embodiments, L⁵ comprises a polyethylene glycol. In some embodiments, L⁵ comprises a modified polyethylene glycol. In some embodiments, L⁵ comprises an amino acid residue. In some embodiments, L⁵ comprises an alkyl group or a substituted alkyl. In some embodiments, L⁵ comprises an aryl group or a substituted aryl group. In some embodiments, L⁵ comprises a diamine (e.g., a linking group comprising an alkylene diamine).

In some embodiments, L⁶ (if present) comprises a polyethylene glycol, a modified polyethylene glycol, an amino acid residue, an alkyl group, a substituted alkyl, an aryl group, a substituted aryl group, or a diamine. In some embodiments, L⁶ comprises a polyethylene glycol. In some embodiments, L⁶ comprises a modified polyethylene glycol. In some embodiments, L⁶ comprises an amino acid residue. In some embodiments, L⁶ comprises an alkyl group or a substituted alkyl. In some embodiments, L⁶ comprises an aryl group or a substituted aryl group. In some embodiments, L⁶ comprises a diamine (e.g., a linking group comprising an alkylene diamine).

In some embodiments, L^{A} is a first linker comprising -(L¹)ₐ-(L²)_{b}-(L³)_{c}-(L⁴)_{d}-(L⁵)ₑ-(L⁶)_{f}-, where:
(L¹)ₐ- is -(T¹-V¹)ₐ—;
(L²)_{b}- is -(T²-V²)_{b}—;
(L³)_{c}- is -(T³-V³)_{c}—;
(L⁴)_{d}- is -(T⁴-V⁴)_{d}—;
(L⁵)ₑ- is -(T⁵-V⁵)ₑ—; and
(L⁶)_{f}- is -(T⁶-V⁶)_{f}—,
wherein T¹, T², T³, T⁴, T⁵ and T⁶, if present, are tether groups; V¹, V², V³, V⁴, V⁵ and V⁶, if present, are covalent bonds or linking functional groups; and
a, b, c, d, e and f are each independently 0 or 1.

In certain embodiments, the sum of a, b, c, d, e and f is 0 to 6. In certain embodiments, the sum of a, b, c, d, e and f is 0. In certain embodiments, the sum of a, b, c, d, e and f is 1. In certain embodiments, the sum of a, b, c, d, e and f is 2. In certain embodiments, the sum of a, b, c, d, e and f is 3. In certain embodiments, the sum of a, b, c, d, e and f is 4. In certain embodiments, the sum of a, b, c, d, e and f is 5. In certain embodiments, the sum of a, b, c, d, e and f is 6. In certain embodiments, a, b, c, d, e and f are each 1. In certain embodiments, a, b, c, d and e are each 1 and f is 0. In certain embodiments, a, b, c and d are each 1 and e and f are each 0. In certain embodiments, a, b, and c are each 1 and d, e and f are each 0. In certain embodiments, a and b are each 1 and c, d, e and f are each 0. In certain embodiments, a is 1 and b, c, d, e and f are each 0.

As described above, in certain embodiments, L¹ is attached to the hydrazinyl-indolyl or a hydrazinyl-pyrrolo-pyridinyl conjugation moiety (e.g., as shown in formula (I) above). As such, in certain embodiments, T¹ is attached to the hydrazinyl-indolyl or a hydrazinyl-pyrrolo-pyridinyl conjugation moiety (e.g., as shown in formula (I) above). In certain embodiments, V¹ is attached to the first drug or active agent. In certain embodiments, L², if present, is attached to the first drug or active agent. As such, in certain embodiments, T², if present, is attached to the first drug or active agent, or V², if present, is attached to the first drug or active agent. In certain embodiments, L³, if present, is attached to the first drug or active agent. As such, in certain embodiments, T³, if present, is attached to the first drug or active agent, or V³, if present, is attached to the first drug or active agent. In certain embodiments, L⁴, if present, is attached to the first drug or active agent. As such, in certain embodiments, T⁴, if present, is attached to the first drug or active agent, or V⁴, if present, is attached to the first drug or active agent. In certain embodiments, L⁵, if present, is attached to the first drug or active agent. As such, in certain embodiments, T₅, if present, is attached to the first drug or active agent, or V⁵, if present, is attached to the first drug or active agent. In certain embodiments, L⁶, if present, is attached to the first drug or active agent. As such, in certain embodiments, T⁶, if present, is attached to the first drug or active agent, or V⁶, if present, is attached to the first drug or active agent.

In certain embodiments, the conjugate of formula (I) includes a second linker, L^{B}. The second linker, L^{B}, may be utilized to bind a second moiety of interest (e.g., a second drug or active agent) to a polypeptide (e.g., an antibody) through a conjugation moiety. The second linker, L^{B}, may be bound (e.g., covalently bonded) to the conjugation moiety (e.g., as described herein). For example, the second linker, L^{B}, may attach a hydrazinyl-indolyl or a hydrazinyl-pyrrolo-pyridinyl conjugation moiety to a second drug. The hydrazinyl-indolyl or hydrazinyl-pyrrolo-pyridinyl conjugation moiety may be used to conjugate the second linker, L^{B}, (and thus the second drug) to a polypeptide, such as an antibody.

For example, as shown in formula (I) above, L^{B} is attached to W³ through a conjugation moiety, and thus W³ is indirectly bonded to the second linker L^{B} through the hydrazinyl-indolyl or a hydrazinyl-pyrrolo-pyridinyl conjugation moiety. As described above, W³ is a polypeptide (e.g., an antibody), and thus L^{B} is attached through the hydrazinyl-indolyl or a hydrazinyl-pyrrolo-pyridinyl conjugation moiety to the polypeptide (antibody), e.g., the linker L^{B} is indirectly bonded to the polypeptide (antibody) through the hydrazinyl-indolyl or a hydrazinyl-pyrrolo-pyridinyl conjugation moiety.

Any convenient linker may be utilized for the second linker L^{B} in the subject conjugates and compounds. In certain embodiments, the second linker L^{B} may include a group selected from alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl. In certain embodiments, the second linker L^{B} may include an alkyl or substituted alkyl group. In certain embodiments, the second linker L^{B} may include an alkenyl or substituted alkenyl group. In certain embodiments, the second linker L^{B} may include an alkynyl or substituted alkynyl group. In certain embodiments, the second linker L^{B} may include an alkoxy or substituted alkoxy group. In certain embodiments, the second linker L^{B} may include an amino or substituted amino group. In certain embodiments, the second linker L^{B} may include a carboxyl or carboxyl ester group. In certain embodiments, the second linker L^{B} may include an acyl amino group. In certain embodiments, the second linker L^{B} may include an alkylamide or substituted alkylamide group. In certain embodiments, the second linker L^{B} may include an aryl or substituted aryl group. In certain embodiments, the second linker L^{B} may include a heteroaryl or substituted heteroaryl group. In certain embodiments, the second linker L^{B} may include a cycloalkyl or substituted cycloalkyl group. In certain embodiments, the second linker L^{B} may include a heterocyclyl or substituted heterocyclyl group.

In certain embodiments, the second linker L^{B} may include a polymer. For example, the polymer may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In certain embodiments, the polymer is a polyalkylene glycol. In certain embodiments, the polymer is a polyethylene glycol. Other linkers are also possible, as shown in the conjugates and compounds described in more detail below.

In some embodiments, L^{B} is a second linker described by the formula:

-(L⁷)_{g}-(L⁸)ₕ-(L⁹)ᵢ-(L¹⁰)ⱼ-(L¹¹)ₖ-(L¹²)-(L¹³)ₘ
wherein L⁷, L⁸, L⁹, L¹⁰, L¹¹, L¹² and L¹³ are each independently a linker subunit, and g, h, i, j, k, l and m are each independently 0 or 1.

In certain embodiments, the sum of g, h, i, j, k, l and m is 0 to 7. In certain embodiments, the sum of g, h, i, j, k, l and m is 0. In certain embodiments, the sum of g, h, i, j, k, l and m is 1. In certain embodiments, the sum of g, h, i, j, k, l and m is 2. In certain embodiments, the sum of g, h, i, j, k, l and m is 3. In certain embodiments, the sum of g, h, i, j, k, l and m is 4. In certain embodiments, the sum of g, h, i, j, k, l and m is 5. In certain embodiments, the sum of g, h, i, j, k, l and m is 6. In certain embodiments, the sum of g, h, i, j, k, l and m is 7. In certain embodiments, g, h, i, j, k, l and m are each 1. In certain embodiments, g, h, i, j, k and 1 are each 1 and m is 0. In certain embodiments, g, h, i, j and k are each 1 and l and m are each 0. In certain embodiments, g, h, i and j are each 1 and k, l and m are each 0. In certain embodiments, g, h, and i are each 1 and j, k, l and m are each 0. In certain embodiments, g and h are each 1 and i, j, k, 1 and m are each 0. In certain embodiments, g is 1 and h, i, j, k, l and m are each 0. In certain embodiments, g, h, i, j, k, l and m are each 0.

In certain embodiments, the linker subunit L⁷ is attached to the hydrazinyl-indolyl or a hydrazinyl-pyrrolo-pyridinyl conjugation moiety (e.g., as shown in formula (I) above). In certain embodiments, the linker subunit L⁸, if present, is attached to the second drug or active agent W². In certain embodiments, the linker subunit L⁹, if present, is attached to the second drug or active agent W². In certain embodiments, the linker subunit L¹⁰, if present, is attached to the second drug or active agent W². In certain embodiments, the linker subunit L¹¹, if present, is attached to the second drug or active agent W². In certain embodiments, the linker subunit L¹², if present, is attached to the second drug or active agent W². In certain embodiments, the linker subunit L¹³, if present, is attached to the second drug or active agent W².

Any convenient linker subunits may be utilized in the second linker L^{B}. Linker subunits of interest include, but are not limited to, units of polymers such as polyethylene glycols, polyethylenes and polyacrylates, amino acid residue(s), carbohydrate-based polymers or carbohydrate residues and derivatives thereof, polynucleotides, alkyl groups, aryl groups, heterocyclic groups, combinations thereof, and substituted versions thereof. In some embodiments, each of L⁷, L⁸, L⁹, L¹⁰, L¹¹, L¹² and L¹³ (if present) comprise one or more groups independently selected from a polyethylene glycol, a modified polyethylene glycol, an amino acid residue, an alkyl group, a substituted alkyl, an aryl group, a substituted aryl group, and a diamine (e.g., a linking group that includes an alkylene diamine).

In some embodiments, L⁷ (if present) comprises a polyethylene glycol, a modified polyethylene glycol, an amino acid residue, an alkyl group, a substituted alkyl, an aryl group, a substituted aryl group, or a diamine. In some embodiments, L⁷ comprises a polyethylene glycol. In some embodiments, L⁷ comprises a modified polyethylene glycol. In some embodiments, L⁷ comprises an amino acid residue. In some embodiments, L⁷ comprises an alkyl group or a substituted alkyl. In some embodiments, L⁷ comprises an aryl group or a substituted aryl group. In some embodiments, L⁷ comprises a diamine (e.g., a linking group comprising an alkylene diamine).

In some embodiments, L⁸ (if present) comprises a polyethylene glycol, a modified polyethylene glycol, an amino acid residue, an alkyl group, a substituted alkyl, an aryl group, a substituted aryl group, or a diamine. In some embodiments, L⁸ comprises a polyethylene glycol. In some embodiments, L⁸ comprises a modified polyethylene glycol. In some embodiments, L⁸ comprises an amino acid residue. In some embodiments, L⁸ comprises an alkyl group or a substituted alkyl. In some embodiments, L⁸ comprises an aryl group or a substituted aryl group. In some embodiments, L⁸ comprises a diamine (e.g., a linking group comprising an alkylene diamine).

In some embodiments, L⁹ (if present) comprises a polyethylene glycol, a modified polyethylene glycol, an amino acid residue, an alkyl group, a substituted alkyl, an aryl group, a substituted aryl group, or a diamine. In some embodiments, L⁹ comprises a polyethylene glycol. In some embodiments, L⁹ comprises a modified polyethylene glycol. In some embodiments, L⁹ comprises an amino acid residue. In some embodiments, L⁹ comprises an alkyl group or a substituted alkyl. In some embodiments, L⁹ comprises an aryl group or a substituted aryl group. In some embodiments, L⁹ comprises a diamine (e.g., a linking group comprising an alkylene diamine).

In some embodiments, L¹⁰ (if present) comprises a polyethylene glycol, a modified polyethylene glycol, an amino acid residue, an alkyl group, a substituted alkyl, an aryl group, a substituted aryl group, or a diamine. In some embodiments, L¹⁰ comprises a polyethylene glycol. In some embodiments, L¹⁰ comprises a modified polyethylene glycol. In some embodiments, L¹⁰ comprises an amino acid residue. In some embodiments, L¹⁰ comprises an alkyl group or a substituted alkyl. In some embodiments, L¹⁰ comprises an aryl group or a substituted aryl group. In some embodiments, L¹⁰ comprises a diamine (e.g., a linking group comprising an alkylene diamine).

In some embodiments, L¹¹ (if present) comprises a polyethylene glycol, a modified polyethylene glycol, an amino acid residue, an alkyl group, a substituted alkyl, an aryl group, a substituted aryl group, or a diamine. In some embodiments, L¹¹ comprises a polyethylene glycol. In some embodiments, L¹¹ comprises a modified polyethylene glycol. In some embodiments, L¹¹ comprises an amino acid residue. In some embodiments, L¹¹ comprises an alkyl group or a substituted alkyl. In some embodiments, L¹¹ comprises an aryl group or a substituted aryl group. In some embodiments, L¹¹ comprises a diamine (e.g., a linking group comprising an alkylene diamine).

In some embodiments, L¹² (if present) comprises a polyethylene glycol, a modified polyethylene glycol, an amino acid residue, an alkyl group, a substituted alkyl, an aryl group, a substituted aryl group, or a diamine. In some embodiments, L¹² comprises a polyethylene glycol. In some embodiments, L¹² comprises a modified polyethylene glycol. In some embodiments, L¹² comprises an amino acid residue. In some embodiments, L¹² comprises an alkyl group or a substituted alkyl. In some embodiments, L¹² comprises an aryl group or a substituted aryl group. In some embodiments, L¹² comprises a diamine (e.g., a linking group comprising an alkylene diamine).

In some embodiments, L¹³ (if present) comprises a polyethylene glycol, a modified polyethylene glycol, an amino acid residue, an alkyl group, a substituted alkyl, an aryl group, a substituted aryl group, or a diamine. In some embodiments, L¹³ comprises a polyethylene glycol. In some embodiments, L¹³ comprises a modified polyethylene glycol. In some embodiments, L¹³ comprises an amino acid residue. In some embodiments, L¹³ comprises an alkyl group or a substituted alkyl. In some embodiments, L¹³ comprises an aryl group or a substituted aryl group. In some embodiments, L¹³ comprises a diamine (e.g., a linking group comprising an alkylene diamine).

In some embodiments, L^{B} is a second linker comprising -(L⁷)_{g}-(L⁸)ₕ-(L⁹)ᵢ-(L¹⁰)ⱼ-(L¹¹)ₖ-(L¹²)ₗ-(L¹³)ₘ-, where:
(L⁷)_{g}- is -(T⁷-V⁷)_{g}—;
(L⁸)ₕ- is -(T⁸-V⁸)ₕ—;
(L⁹)ᵢ- is -(T⁹-V⁹)ᵢ—;
(L¹⁰)ⱼ- is -(T¹⁰-V¹⁰)ⱼ—;
(L¹¹)ₖ- is -(T¹¹-V¹¹)ₖ—;
(L¹²)ₗ- is -(T¹²-V¹²)ₗ—; and
(L³)ₘ- is -(T¹³-V¹³)ₘ—,
wherein T⁷, T⁸, T⁹, T¹⁰, T¹¹, T¹² and T¹³, if present, are tether groups;
V⁷, V⁸, V⁹, V¹⁰, V¹¹, V¹² and V¹³, if present, are covalent bonds or linking functional groups; and
g, h, i, j, k, l and m are each independently 0 or 1.

In certain embodiments, the sum of g, h, i, j, k, l and m is 0 to 7. In certain embodiments, the sum of g, h, i, j, k, l and m is 0. In certain embodiments, the sum of g, h, i, j, k, l and m is 1. In certain embodiments, the sum of g, h, i, j, k, l and m is 2. In certain embodiments, the sum of g, h, i, j, k, l and m is 3. In certain embodiments, the sum of g, h, i, j, k, l and m is 4. In certain embodiments, the sum of g, h, i, j, k, l and m is 5. In certain embodiments, the sum of g, h, i, j, k, l and m is 6. In certain embodiments, the sum of g, h, i, j, k, l and m is 7. In certain embodiments, g, h, i, j, k, l and m are each 1. In certain embodiments, g, h, i, j, k and 1 are each 1 and m is 0. In certain embodiments, g, h, i, j and k are each 1 and l and m are each 0. In certain embodiments, g, h, i and j are each 1 and k, l and m are each 0. In certain embodiments, g, h, and i are each 1 and j, k, l and m are each 0. In certain embodiments, g and h are each 1 and i, j, k, 1 and m are each 0. In certain embodiments, g is 1 and h, i, j, k, l and m are each 0. In certain embodiments, g, h, i, j, k, l and m are each 0.

As described above, in certain embodiments, L⁷ is attached to the hydrazinyl-indolyl or a hydrazinyl-pyrrolo-pyridinyl conjugation moiety (e.g., as shown in formula (I) above). As such, in certain embodiments, T⁷ is attached to the hydrazinyl-indolyl or a hydrazinyl-pyrrolo-pyridinyl conjugation moiety (e.g., as shown in formula (I) above). In certain embodiments, V⁷ is attached to the second drug or active agent. In certain embodiments, L⁸, if present, is attached to the second drug or active agent. As such, in certain embodiments, T⁸, if present, is attached to the second drug or active agent, or V⁸, if present, is attached to the second drug or active agent. In certain embodiments, L⁹, if present, is attached to the second drug or active agent. As such, in certain embodiments, T⁹, if present, is attached to the second drug or active agent, or V⁹, if present, is attached to the second drug or active agent. In certain embodiments, L¹⁰, if present, is attached to the second drug or active agent. As such, in certain embodiments, T¹⁰, if present, is attached to the second drug or active agent, or V10⁴, if present, is attached to the second drug or active agent. In certain embodiments, L¹¹, if present, is attached to the second drug or active agent. As such, in certain embodiments, T¹¹, if present, is attached to the second drug or active agent, or V¹¹, if present, is attached to the second drug or active agent. In certain embodiments, L¹², if present, is attached to the second drug or active agent. As such, in certain embodiments, T¹², if present, is attached to the second drug or active agent, or V¹², if present, is attached to the second drug or active agent. In certain embodiments, L¹³, if present, is attached to the second drug or active agent. As such, in certain embodiments, T¹³, if present, is attached to the second drug or active agent, or V¹³, if present, is attached to the second drug or active agent.

Regarding the tether groups, T¹, T², T³, T⁴, T₅, T⁶, T⁷, T⁸, T⁹, T¹⁰, T¹¹, T¹² and T¹³, any convenient tether groups may be utilized in the subject linkers. In some embodiments, T¹, T², T³, T⁴, T⁵, T⁶, T⁷, T⁸, T⁹, T¹⁰, T¹¹, T¹² and T¹³ each comprise one or more groups independently selected from a covalent bond, a (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, where each w is an integer from 1 to 20, each n is an integer from 1 to 30, each p is an integer from 1 to 20, and each x is an integer from 1 to 12.

In certain embodiments, the tether group (e.g., T¹, T², T³, T⁴, T⁵, T⁶, T⁷, T⁸, T⁹, T¹⁰, T¹¹, T¹² and/or T¹³) includes a (C₁-C₁₂)alkyl or a substituted (C₁-C₁₂)alkyl. In certain embodiments, (C₁-C₁₂)alkyl is a straight chain or branched alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, (C₁-C₁₂)alkyl may be an alkyl or substituted alkyl, such as C₁-C₁₂ alkyl, or C₁-C₁₀ alkyl, or C₁-C₆ alkyl, or C₁-C₃ alkyl. In some instances, (C₁-C₁₂)alkyl is a C₂-alkyl. For example, (C₁-C₁₂)alkyl may be an alkylene or substituted alkylene, such as C₁-C₁₂ alkylene, or C₁-C₁₀ alkylene, or C₁-C₆ alkylene, or C₁-C₃ alkylene. In some instances, (C₁-C₁₂)alkyl is a C₁-alkylene (e.g., CH₂). In some instances, (C₁-C₁₂)alkyl is a C₂-alkylene (e.g., CH₂CH₂). In some instances, (C₁-C₁₂)alkyl is a C₃-alkylene (e.g., CH₂CH₂CH₂).

In certain embodiments, substituted (C₁-C₁₂)alkyl is a straight chain or branched substituted alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, substituted (C₁-C₁₂)alkyl may be a substituted alkyl, such as substituted C₁-C₁₂ alkyl, or substituted C₁-C₁₀ alkyl, or substituted C₁-C₆ alkyl, or substituted C₁-C₃ alkyl. In some instances, substituted (C₁-C₁₂)alkyl is a substituted C₂-alkyl. For example, substituted (C₁-C₁₂)alkyl may be a substituted alkylene, such as substituted C₁-C₁₂ alkylene, or substituted C₁-C₁₀ alkylene, or substituted C₁-C₆ alkylene, or substituted C₁-C₃ alkylene. In some instances, substituted (C₁-C₁₂)alkyl is a substituted C₁-alkylene (e.g., C₁-alkylene substituted with —SO₃H). In some instances, substituted (C₁-C₁₂)alkyl is a substituted C₂-alkylene. In some instances, substituted (C₁-C₁₂)alkyl is a substituted C₃-alkylene. For example, substituted (C₁-C₁₂)alkyl may include C₁-C₁₂ alkylene (e.g., C₃-alkylene or C₅-alkylene) substituted with a (PEG)ₖ group as described herein (e.g., —CONH(PEG)ₖ, such as —CONH(PEG)₃ or —CONH(PEG)₅; or —NHCO(PEG)ₖ, such as —NHCO(PEG)₇), or may include C₁-C₁₂ alkylene (e.g., C₃-alkylene) substituted with a —CONHCH₂CH₂SO₃H group, or may include C₁-C₁₂ alkylene (e.g., C₅-alkylene) substituted with a —NHCOCH₂SO₃H group.

In certain embodiments, the tether group (e.g., T¹, T², T³, T⁴, T⁵, T⁶, T⁷, T⁸, T⁹, T¹⁰, T¹¹ T¹² and/or T¹³) includes an aryl, substituted aryl, heteroaryl, substituted heteroaryl, cycloalkyl, substituted cycloalkyl, heterocyclyl, or substituted heterocyclyl. In some instances, the tether group (e.g., T¹, T², T³, T⁴, T⁵, T⁶, T⁷, T⁸, T⁹, T¹⁰, T¹¹, T¹² and/or T¹³) includes an aryl or substituted aryl. For example, the aryl can be phenyl. In some cases, the substituted aryl is a substituted phenyl. The substituted phenyl can be substituted with one or more substituents selected from (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl. In some instances, the substituted aryl is a substituted phenyl, where the substituent includes a cleavable moiety as described herein (e.g., an enzymatically cleavable moiety, such as a glycoside or glycoside derivative).

In some instances, the tether group (e.g., T¹, T², T³, T⁴, T⁵, T⁶, T⁷, T⁸, T⁹, T¹⁰, T¹¹, T¹² and/or T¹³) includes a heteroaryl or substituted heteroaryl, such triazolyl (e.g., 1,2,3-triazolyl). In some instances, the tether group (e.g., T¹, T², T³, T⁴, T⁵, T⁶, T⁷, T⁸, T⁹, T¹⁰, T¹¹, T¹² and/or T¹³) includes a cycloalkyl or substituted cycloalkyl. In some instances, the tether group (e.g., T¹, T², T³, T⁴, T⁵, T⁶, T⁷, T⁸, T⁹, T¹⁰, T¹¹, T¹² and/or T¹³) includes a heterocyclyl or substituted heterocyclyl. In some instances, the substituent on the substituted heteroaryl, substituted cycloalkyl or substituted heterocyclyl includes a cleavable moiety as described herein (e.g., an enzymatically cleavable moiety, such as a glycoside or glycoside derivative).

In certain embodiments, the tether group (e.g., T¹, T², T³, T⁴, T⁵, T⁶, T⁷, T⁸, T⁹, T¹⁰, T¹¹, T¹² and/or T¹³) includes an ethylene diamine (EDA) moiety, e.g., an EDA containing tether group. In certain embodiments, (EDA)_{W} includes one or more EDA moieties, such as where w is an integer from 1 to 50, such as from 1 to 40, from 1 to 30, from 1 to 20, from 1 to 12 or from 1 to 6, such as 1, 2, 3, 4, 5 or 6). The linked ethylene diamine (EDA) moieties may optionally be substituted at one or more convenient positions with any convenient substituents, e.g., with an alkyl, a substituted alkyl, an acyl, a substituted acyl, an aryl or a substituted aryl. In certain embodiments, the EDA moiety is described by the structure:

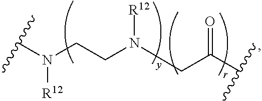

where y is an integer from 1 to 6, or is 0 or 1, and each R¹² is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, y is 1, 2, 3, 4, 5 or 6. In certain embodiments, y is 1 and r is 0. In certain embodiments, y is 1 and r is 1. In certain embodiments, y is 2 and r is 0. In certain embodiments, y is 2 and r is 1. In certain embodiments, each R¹² is independently selected from hydrogen, an alkyl, a substituted alkyl, an aryl and a substituted aryl. In certain embodiments, any two adjacent R¹² groups of the EDA may be cyclically linked, e.g., to form a piperazinyl ring. In certain embodiments, y is 1 and the two adjacent R¹² groups are an alkyl group, cyclically linked to form a piperazinyl ring. In certain embodiments, y is 1 and the adjacent R¹² groups are selected from hydrogen, an alkyl (e.g., methyl) and a substituted alkyl (e.g., lower alkyl-OH, such as ethyl-OH or propyl-OH).

In certain embodiments, the tether group (e.g., T¹, T², T³, T⁴, T₅, T⁶, T⁷, T⁸, T⁹, T¹⁰, T¹¹, T¹² and/or T¹³) includes a 4-amino-piperidine (4AP) moiety (also referred to herein as piperidin-4-amino, P4A). The 4AP moiety may optionally be substituted at one or more convenient positions with any convenient substituents, e.g., with an alkyl, a substituted alkyl, a polyethylene glycol moiety, an acyl, a substituted acyl, an aryl or a substituted aryl. In certain embodiments, the 4AP moiety is described by the structure:

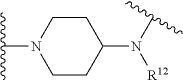

where R¹² is selected from hydrogen, alkyl, substituted alkyl, a polyethylene glycol moiety (e.g., a polyethylene glycol or a modified polyethylene glycol), alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹² is a polyethylene glycol moiety. In certain embodiments, R¹² is a carboxy modified polyethylene glycol.

In certain embodiments, R¹² includes a polyethylene glycol moiety described by the formula: (PEG)ₖ, which may be represented by the structure:

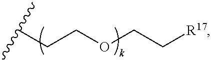

where k is an integer from 1 to 20, such as from 1 to 18, or from 1 to 16, or from 1 to 14, or from 1 to 12, or from 1 to 10, or from 1 to 8, or from 1 to 6, or from 1 to 4, or 1 or 2, such as 1, 2, 3, 4, 5, 6, 7, 8, 9, 10, 11, 12, 13, 14, 15, 16, 17, 18, 19 or 20. In some instances, k is 2. In certain embodiments, R¹⁷ is selected from OH, COOH, OR, or COOR, where R is selected from alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl. In certain embodiments, R¹⁷ is COOH. In certain embodiments, R¹⁷ is OH. In certain embodiments, R¹⁷ is OCH₃.

In certain embodiments, a tether group (e.g., T¹, T², T³, T⁴, T⁵, T⁶, T⁷, T⁸, T⁹, T¹⁰, T¹¹, T¹² and/or T¹³) includes (PEG)ₙ, where (PEG)ₙ is a polyethylene glycol or a modified polyethylene glycol linking unit. In certain embodiments, (PEG)ₙ is described by the structure:

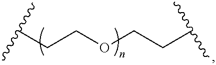

where n is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In some instances, n is 2. In some instances, n is 3. In some instances, n is 6. In some instances, n is 12.

In certain embodiments, a tether group (e.g., T¹, T², T³, T⁴, T⁵, T⁶, T⁷, T⁸, T⁹, T¹⁰, T¹¹, T¹² and/or T¹³) includes (AA)ₚ, where AA is an amino acid residue. Any convenient amino acids may be utilized. Amino acids of interest include but are not limited to, L- and D-amino acids, naturally occurring amino acids such as any of the 20 primary alpha-amino acids and beta-alanine, non-naturally occurring amino acids (e.g., amino acid analogs), such as a non-naturally occurring alpha-amino acid or a non-naturally occurring beta-amino acid, etc. In certain embodiments, p is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In certain embodiments, p is 1. In certain embodiments, p is 2.

In certain embodiments, a tether group (e.g., T¹, T², T³, T⁴, T⁵, T⁶, T⁷, T⁸, T⁹, T¹⁰, T¹¹, T¹² and/or T¹³) includes an amino acid analog. Amino acid analogs include compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as C₁ or Br, etc.), deletion of a group, substitution of a covalent bond (single bond for double bond, etc.), or combinations thereof. For example, amino acid analogs may include α-hydroxy acids, and α-amino acids, and the like. Examples of amino acid analogs include, but are not limited to, sulfoalanine, and the like.

In certain embodiments, a tether group (e.g., T¹, T², T³, T⁴, T⁵, T⁶, T⁷, T⁵, T⁹, T¹⁰, T¹¹, T¹² and/or T¹³) includes a moiety described by the formula —(CR¹³OH)ₓ—, where x is 0 or x is an integer from 1 to 50, such as from 1 to 40, from 1 to 30, from 1 to 20, from 1 to 12 or from 1 to 6, such as 1, 2, 3, 4, 5, 6, 7, 8, 9, 10, 11 or 12. In certain embodiments, x is 1. In certain embodiments, x is 2. In certain embodiments, R¹³ is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹³ is hydrogen. In certain embodiments, R¹³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹³ is alkynyl or substituted alkynyl. In certain embodiments, R¹³ is alkoxy or substituted alkoxy. In certain embodiments, R¹³ is amino or substituted amino. In certain embodiments, R¹³ is carboxyl or carboxyl ester. In certain embodiments, R¹³ is acyl or acyloxy. In certain embodiments, R¹³ is acyl amino or amino acyl. In certain embodiments, R¹³ is alkylamide or substituted alkylamide. In certain embodiments, R¹³ is sulfonyl. In certain embodiments, R¹³ is thioalkoxy or substituted thioalkoxy. In certain embodiments, R¹³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹³ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₈ heterocyclyl or C₃₋₅ substituted heterocyclyl.

In certain embodiments, R¹³ is selected from hydrogen, alkyl, substituted alkyl, aryl, and substituted aryl. In these embodiments, alkyl, substituted alkyl, aryl, and substituted aryl are as described above for R¹³.

In certain embodiments, the tether group (e.g., T¹, T², T³, T⁴, T⁵, T⁶, T⁷, T⁸, T⁹, T¹⁰, T¹¹, T¹² and/or T¹³) includes an acetal group, a disulfide, a hydrazine, or an ester. In some embodiments, the tether group includes an acetal group. In some embodiments, the tether group includes a hydrazine. In some embodiments, the tether group includes a disulfide. In some embodiments, the tether group includes an ester.

In certain embodiments, a tether group (e.g., T¹, T², T³, T⁴, T⁵, T⁶, T⁷, T⁸, T⁹, T¹⁰, T¹¹, T¹² and/or T¹³) includes a meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), or para-hydroxy-phenyl (PHP).

In some embodiments, a tether group includes a MABO group described by the following structure:

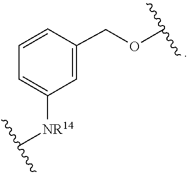

In some embodiments, a tether group includes a MABC group described by the following structure:

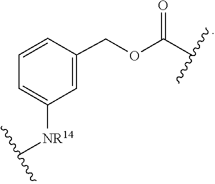

In some embodiments, a tether group includes a PABO group described by the following structure:

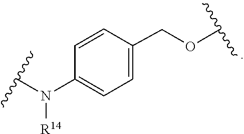

In some embodiments, a tether group includes a PABC group described by the following structure:

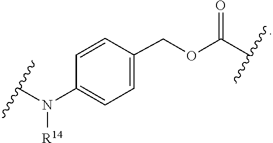

In some embodiments, a tether group includes a PAB group described by the following structure:

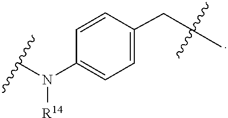

In some embodiments, a tether group includes a PABA group described by the following structure:

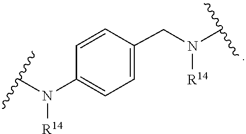

In some embodiments, a tether group includes a PAP group described by the following structure:

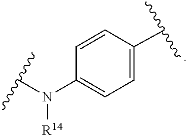

In some embodiments, a tether group includes a PHP group described by the following structure:

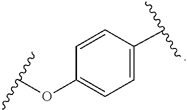 [SMILES: *Oc1ccc(*)cc1]

In certain embodiments, each R¹⁴ is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, R¹⁴ is hydrogen. In certain embodiments, each R¹⁴ is hydrogen. In certain embodiments, R¹⁴ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹⁴ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹⁴ is alkynyl or substituted alkynyl. In certain embodiments, R¹⁴ is alkoxy or substituted alkoxy. In certain embodiments, R¹⁴ is amino or substituted amino. In certain embodiments, R¹⁴ is carboxyl or carboxyl ester. In certain embodiments, R¹⁴ is acyl or acyloxy. In certain embodiments, R¹⁴ is acyl amino or amino acyl. In certain embodiments, R¹⁴ is alkylamide or substituted alkylamide. In certain embodiments, R¹⁴ is sulfonyl. In certain embodiments, R¹⁴ is thioalkoxy or substituted thioalkoxy. In certain embodiments, R¹⁴ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹⁴ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹⁴ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹⁴ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In some embodiments of the MABO, MABC, PABO, PABC, PAB, PABA, PAP, and PHP tether structures shown above, the phenyl ring may be substituted with one or more additional groups selected from halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, one or more of the tether groups T¹, T², T³, T⁴, T⁵, T⁶, T⁷, T⁸, T⁹, T¹⁰, T¹¹, T¹² and/or T¹³ is each optionally substituted with a glycoside or glycoside derivative. For example, in some instances, T¹, T², T³, T⁴, T⁵ and T⁶ are each optionally substituted with a glycoside. In some instances, T⁷, T⁸, T⁹, T¹⁰, T¹¹, T¹² and T¹³ are each optionally substituted with a glycoside. In certain embodiments, the glycoside or glycoside derivative is selected from a glucuronide, a galactoside, a glucoside, a mannoside, a fucoside, 0-GlcNAc, and O-GalNAc.

In certain embodiments, the MABO, MABC, PABO, PABC, PAB, PABA, PAP, and PHP tether structures shown above may be substituted with an one or more additional groups selected from a glycoside and a glycoside derivative. For example, in some embodiments of the MABO, MABC, PABO, PABC, PAB, PABA, PAP, and PHP tether structures shown above, the phenyl ring may be substituted with one or more additional groups selected from a glycoside and a glycoside derivative. In certain embodiments, the glycoside or glycoside derivative is selected from a glucuronide, a galactoside, a glucoside, a mannoside, a fucoside, O-GlcNAc, and 0-GalNAc.

For example, in some embodiments, the glycoside or glycoside derivative can be selected from the following structures:

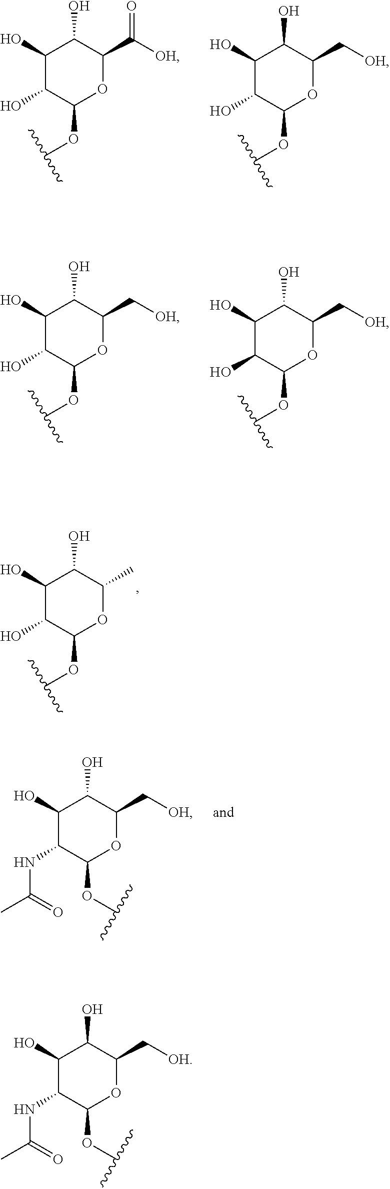

Regarding the linking functional groups, V¹, V², V³, V⁴, V⁵, V⁶, V⁷, V⁸, V⁹, V¹⁰, V¹¹, V¹² and V¹³ any convenient linking functional groups may be utilized in the subject linkers. Linking functional groups of interest include, but are not limited to, amino, carbonyl, amido, oxycarbonyl, carboxy, sulfonyl, sulfoxide, sulfonylamino, aminosulfonyl, thio, oxy, phospho, phosphoramidate, thiophosphoraidate, and the like. In some embodiments, V¹, V², V³, V⁴, V⁵, V⁶, V⁷, V⁸, V⁹, V¹⁰, V¹¹, V¹² and V¹³ are each independently selected from a covalent bond, —CO—, —NR¹⁵, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, where q is an integer from 1 to 6. In certain embodiments, q is an integer from 1 to 6 (e.g., 1, 2, 3, 4, 5 or 6). In certain embodiments, q is 1. In certain embodiments, q is 2. In certain embodiments, q is 3. In certain embodiments, q is 4. In certain embodiments, q is 5. In certain embodiments, q is 6.

In some embodiments, each R¹⁵ is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, R¹⁵ is hydrogen. In certain embodiments, each R¹⁵ is hydrogen. In certain embodiments, R¹⁵ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹⁵ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹⁵ is alkynyl or substituted alkynyl. In certain embodiments, R¹⁵ is alkoxy or substituted alkoxy. In certain embodiments, R¹⁵ is amino or substituted amino. In certain embodiments, R¹⁵ is carboxyl or carboxyl ester. In certain embodiments, R¹⁵ is acyl or acyloxy. In certain embodiments, R¹⁵ is acyl amino or amino acyl. In certain embodiments, R¹⁵ is alkylamide or substituted alkylamide. In certain embodiments, R¹⁵ is sulfonyl. In certain embodiments, R¹⁵ is thioalkoxy or substituted thioalkoxy. In certain embodiments, R¹⁵ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹⁵ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹⁵ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹⁵ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl. In these embodiments, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl are as described above for R¹⁵.

As described above, in some embodiments, L^{A} is a first linker comprising -(T¹-V¹)ₐ-(T²-V²)_{b}-(T³-V³)_{c}-(T⁴-V⁴)ₐ-(T⁵-V⁵)ₑ-(T⁶-V⁶)_{f}—, where a, b, c, d, e and f are each independently 0 or 1.

In some embodiments, in the first linker L^{A}:
T¹ is selected from a (C₁-C₁₂)alkyl and a substituted (C₁-C₁₂)alkyl;
T², T³, T⁴, T⁵ and T⁶ are each independently selected from (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PHP, an acetal group, a disulfide, a hydrazine, and an ester; and
V¹, V², V³, V⁴, V⁵ and V⁶ are each independently selected from a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein q is an integer from 1 to 6;
wherein:
(PEG)ₙ is

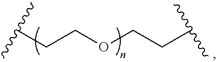

where n is an integer from 1 to 30;
EDA is an ethylene diamine moiety having the following structure:

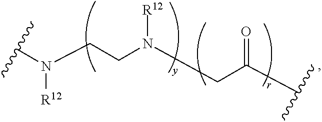

where y is an integer from 1 to 6 and r is 0 or 1;
4-amino-piperidine (4AP) is

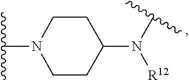

AA is an amino acid residue, where p is an integer from 1 to 20; and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring;
each R¹³ is independently selected from hydrogen, alkyl, substituted alkyl, aryl, and substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

In certain embodiments, T¹, T², T³, T⁴, T⁵ and T⁶ and V¹, V², V³, V⁴, V⁵ and V⁶ are selected from the following:
wherein:
   T¹ is (C₁-C₁₂)alkyl and V¹ is —CO—;
   T² is AA and V² is absent;
   T³ is PABC and V³ is absent; and
   d, e and f are each 0; or
wherein:
   T¹ is (C₁-C₁₂)alkyl and V¹ is —CONH—;
   T² is (PEG)ₙ and V² is —CO—;
   T³ is AA and V³ is absent;
   T⁴ is PABC and V⁴ is absent; and
   e and f are each 0; or
wherein:
   T¹ is (C₁-C₁₂)alkyl and V¹ is —CO—;
   T² is an amino acid analog and V² is —NH—;
   T³ is (PEG)ₙ and V³ is —CO—;
   T⁴ is AA and V⁴ is absent;
   T⁵ is PABC and V⁵ is absent; and
   f is 0; or
wherein:
   T¹ is (C₁-C₁₂)alkyl and V¹ is —CONH—;
   T² is (PEG)ₙ and V² is —CO—;
   T³ is AA and V³ is absent;
   T⁴ is PABC and V⁴ is absent; and
   e and f are each 0; or
wherein:
   T¹ is (C₁-C₁₂)alkyl and V¹ is —CONH—;
   T² is substituted (C₁-C₁₂)alkyl and V² is —CO—;
   T³ is AA and V³ is absent;
   T⁴ is PABC and V⁴ is absent; and
   e and f are each 0; or
wherein:
   T¹ is (C₁-C₁₂)alkyl and V¹ is —CONH—;
   T² is (PEG)ₙ and V² is —CO—;
   T³ is AA and V³ is absent;
   T⁴ is PABA and V⁴ is —CO—;
   T⁵ is (C₁-C₁₂)alkyl and V⁵ is absent; and
   f is 0; or
wherein:
   T¹ is (C₁-C₁₂)alkyl and V¹ is —CO—;
   T² is 4AP and V² is —CO—;
   T³ is (C₁-C₁₂)alkyl and V³ is —CO—;
   T⁴ is AA and V⁴ is absent;
   T⁵ is PABC and V⁵ is absent; and
   f is 0; or
wherein:
   T¹ is (C₁-C₁₂)alkyl and V¹ is —CO—;
   T² is 4AP and V² is —CO—;
   T³ is (C₁-C₁₂)alkyl and V³ is —O—;
   T⁴ is (C₁-C₁₂)alkyl and V⁴ is —CO—;
   T⁵ is AA and V⁵ is absent; and
   T⁶ is PABC and V⁶ is absent; or
wherein:
   T¹ is (C₁-C₁₂)alkyl and V¹ is —CO—;
   T² is an amino acid analog and V² is absent;
   T³ is AA and V³ is absent;
   T⁴ is PABC and V⁴ is absent; and
   e and f are each 0; or
wherein:
   T¹ is (C₁-C₁₂)alkyl and V¹ is —CONH—;
   T² is (PEG)ₙ and V² is —CONH—;
   T³ is substituted (C₁-C₁₂)alkyl and V³ is —CO—;
   T⁴ is AA and V⁴ is absent;
   T⁵ is PABC and V⁵ is absent; and
   f is 0; or
wherein:
   T¹ is (C₁-C₁₂)alkyl and V¹ is —CO—;
   T² is an AA and V² is —NH—;
   T³ is (PEG)ₙ and V³ is —CO—;
   T⁴ is AA and V⁴ is absent;
   T⁵ is PABC and V⁵ is absent; and
   f is 0; or
wherein:
   T¹ is (C₁-C₁₂)alkyl and V¹ is —CONH—;
   T² is (PEG)ₙ and V² is —CO—;
   T³ is AA and V³ is absent;
   T⁴ is PAP and V⁴ is —C(O)O—; and
   e and f are each 0; or
wherein:
   T¹ is (C₁-C₁₂)alkyl and V¹ is —CONH—;
   T² is substituted (C₁-C₁₂)alkyl and V² is —CO—;
   T³ is AA and V³ is absent;
   T⁴ is PABC and V⁴ is absent; and
   e and f are each 0; or
wherein:
   T¹ is (C₁-C₁₂)alkyl and V¹ is —CONH—;
   T² is substituted (C₁-C₁₂)alkyl and V² is —CO—;
   T³ is PABC and V³ is absent; and
   d, e and f are each 0.

In certain embodiments, the left-hand side of the above linker structure for the first linker L^{A} is attached to the hydrazinyl-indolyl or a hydrazinyl-pyrrolo-pyridinyl conjugation moiety, and the right-hand side of the above linker structure for the first linker L^{A} is attached to the first drug or active agent.

As described above, in some embodiments, L^{B} is a second linker comprising -(T⁷-V⁷)_{g}-(T⁸-V⁸)ₕ-(T⁹-V⁹)ᵢ-(T¹⁰-V¹⁰)ⱼ-(T¹¹-V¹¹)ₖ(T¹²-V¹²)ₗ-(T¹³-V¹³)ₘ—, where g, h, i, j, k, l and m are each independently 0 or 1.

In some embodiments, in the second linker L^{B}:
T⁷ is selected from a (C₁-C₁₂)alkyl and a substituted (C₁-C₁₂)alkyl;
T⁸, T⁹, T¹⁰, T¹¹, T¹² and T¹³ are each independently selected from (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PHP, an acetal group, a disulfide, a hydrazine, and an ester; and
V⁷, V⁸, V⁹, V¹⁰, V¹¹, V¹² and V¹³ are each independently selected from a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR″ ⁵SO₂— and —P(O)OH—, wherein q is an integer from 1 to 6;
wherein:
(PEG)ₙ is

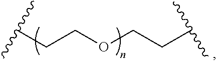

where n is an integer from 1 to 30;
EDA is an ethylene diamine moiety having the following structure:

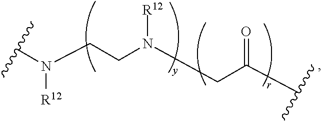

where y is an integer from 1 to 6 and r is 0 or 1;
4-amino-piperidine (4AP) is

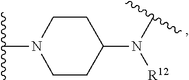

AA is an amino acid residue, where p is an integer from 1 to 20; and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring;
each R¹³ is independently selected from hydrogen, alkyl, substituted alkyl, aryl, and substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

Any convenient tether groups may be utilized for T⁷, T⁸, T⁹, T¹⁰, T¹¹, T¹² and T¹³. For example, any of the tether groups described above in relation to T¹, T², T³, T⁴, T⁵ and T⁶ may be used for the tether groups T⁷, T⁸, T⁹, T¹⁰, T¹¹, T¹² and T¹³.

Any convenient linking functional groups may be utilized for V⁷, V⁸, V⁹, V¹⁰, V¹¹, V¹² and V¹³. For example, any of the linking functional groups described above in relation to V¹, V², V³, V⁴, V⁵ and V⁶ may be used for the linking functional groups V⁷, V⁸, V⁹, V¹⁰, V¹¹, V¹² and V³.

In certain embodiments, each R¹³ is independently selected from hydrogen, alkyl, substituted alkyl, aryl, and substituted aryl. In these embodiments, alkyl, substituted alkyl, aryl, and substituted aryl are as described above for R¹³.

In certain embodiments, each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl. In these embodiments, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl are as described above for R¹⁵. In these embodiments, various possible substituents are as described above for R¹.

In certain embodiments of the second linker L^{B}, one or more of the tether groups T⁷, T⁸, T⁹, T¹⁰, T¹¹, T¹² and T¹³ is each optionally substituted with a glycoside or glycoside derivative. In certain embodiments, the glycoside or glycoside derivative is selected from a glucuronide, a galactoside, a glucoside, a mannoside, a fucoside, O-GlcNAc, and O-GalNAc.

In certain embodiments of the second linker L^{B}, the MABO, MABC, PABO, PABC, PAB, PABA, PAP, and PHP tether structures shown above may be substituted with an one or more additional groups selected from a glycoside and a glycoside derivative. For example, in some embodiments of the MABO, MABC, PABO, PABC, PAB, PABA, PAP, and PHP tether structures shown above, the phenyl ring may be substituted with one or more additional groups selected from a glycoside and a glycoside derivative. In certain embodiments, the glycoside or glycoside derivative is selected from a glucuronide, a galactoside, a glucoside, a mannoside, a fucoside, O-GlcNAc, and O-GalNAc.

In certain embodiments, T⁷, T⁸, T⁹, T¹⁰, T¹¹, T¹² and T¹³ and V⁷, V⁸, V⁹, V¹⁰, V¹¹, V¹² and V¹³ are selected from the following:
wherein:
   T⁷ is absent and V⁷ is —NHCO—;
   T⁸ is (C₁-C₁₂)alkyl and V⁸ is —CO—;
   T⁹ is AA and V⁹ is absent;
   T¹⁰ is PABC and V¹⁰ is absent; and
   k, l and m are each 0; or
wherein:
   T⁷ is absent and V⁷ is —NHCO—;
   T⁸ is (C₁-C₁₂)alkyl and V⁸ is —CONH—;
   T⁹ is (PEG)ₙ and V⁹ is —CO—;
   T¹⁰ is AA and V¹⁰ is absent; and
   T¹¹ is PABC and V¹¹ is absent; and
   l and m are each 0; or
wherein:
   T⁷ is absent and V⁷ is —NHCO—;
   T⁸ is (C₁-C₁₂)alkyl and V⁸ is —CO—;
   T⁹ is an amino acid analog and V⁹ is —NH—;
   T¹⁰ is (PEG)ₙ and V¹⁰ is —CO—;
   T¹¹ is AA and V¹¹ is absent;
   T¹² is PABC and V¹² is absent; and
   m is 0; or
wherein:
   T⁷ is absent and V⁷ is —NHCO—;
   T⁸ is (C₁-C₁₂)alkyl and V⁸ is —CONH—;
   T⁹ is (PEG)ₙ and V⁹ is —CO—;
   T¹⁰ is AA and V¹⁰ is absent;
   T¹¹ is PABC and V¹¹ is absent; and
   l and m are each 0; or
wherein:
   T⁷ is absent and V⁷ is —NHCO—;
   T⁸ is (C₁-C₁₂)alkyl and V⁸ is —CONH—;
   T⁹ is substituted (C₁-C₁₂)alkyl and V⁹ is —CO—;
   T¹⁰ is AA and V¹⁰ is absent;
   T¹¹ is PABC and V¹¹ is absent; and
   l and m are each 0; or
wherein:
   T⁷ is absent and V⁷ is —NHCO—;
   T⁸ is (C₁-C₁₂)alkyl and V⁸ is —CONH—;
   T⁹ is (PEG)ₙ and V⁹ is —CO—;
   T¹⁰ is AA and V¹⁰ is absent;
   T¹¹ is PABA and V¹¹ is —CO—;
   T¹² is (C₁-C₁₂)alkyl and V¹² is absent; and
   m is 0; or
wherein:
   T⁷ is absent and V⁷ is —NHCO—;
   T⁸ is (C₁-C₁₂)alkyl and V⁸ is —CO—;
   T⁹ is 4AP and V⁹ is —CO—;
   T¹⁰ is (C₁-C₁₂)alkyl and V¹⁰ is —CO—;
   T¹¹ is AA and V¹¹ is absent;
   T¹² is PABC and V¹² is absent; and
   m is 0; or
wherein:
   T⁷ is absent and V⁷ is —NHCO—;
   T⁸ is (C₁-C₁₂)alkyl and V⁸ is —CO—;
   T⁹ is 4AP and V⁹ is —CO—;
   T¹⁰ is (C₁-C₁₂)alkyl and V¹⁰ is —O—;
   T¹¹ is (C₁-C₁₂)alkyl and V¹¹ is —CO—;
   T¹² is AA and V¹² is absent; and
   T¹³ PABC and V¹³ is absent; or
wherein:
   T⁷ is absent and V⁷ is —NHCO—;
   T⁸ is (C₁-C₁₂)alkyl and V⁸ is —CO—;
   T⁹ is an amino acid analog and V⁹ is absent;
   T¹⁰ is AA and V¹⁰ is absent;
   T¹¹ is PABC and V¹¹ is absent; and
   l and m are each 0; or
wherein:
   T⁷ is absent and V⁷ is —NHCO—;
   T⁸ is (C₁-C₁₂)alkyl and V⁸ is —CONH—;
   T⁹ is (PEG)ₙ and V⁹ is —CONH—;
   T¹⁰ is substituted (C₁-C₁₂)alkyl and V¹⁰ is —CO—;
   T¹¹ is AA and V¹¹ is absent;
   T¹² is PABC and V¹² is absent; and
   m is 0; or
wherein:
   T⁷ is absent and V⁷ is —NHCO—;
   T⁸ is (C₁-C₁₂)alkyl and V⁸ is —CO—;
   T⁹ is AA and V⁹ is —NH—;
   T¹⁰ is (PEG)ₙ and V¹⁰ is —CO—;
   T¹¹ is AA and V¹¹ is absent;
   T¹² is PABC and V¹² is absent; and
   m is 0; or
wherein:
   T⁷ is absent and V⁷ is —NHCO—;
   T⁸ is (C₁-C₁₂)alkyl and V⁸ is —CONH—;
   T⁹ is (PEG)ₙ and V⁹ is —CO—;
   T¹⁰ is AA and V¹⁰ is absent;
   T¹¹ is PAP and V¹¹ is —C(O)O—; and
   l and m are each 0; or
wherein:
   T⁷ is absent and V⁷ is —NHCO—;
   T⁸ is (C₁-C₁₂)alkyl and V⁸ is —CO—;
   T⁹ is AA and V⁹ is absent;
   T¹⁰ is PABC and V¹⁰ is absent;
   T¹¹ is PAP and V¹¹ is —C(O)O—; and
   l and m are each 0; or
wherein:
   T⁷ is absent and V⁷ is —NHCO—;
   T⁸ is (C₁-C₁₂)alkyl and V⁸ is —CONH—;
   T⁹ is substituted (C₁-C₁₂)alkyl and V⁹ is —CO—;
   T¹⁰ is PABC and V¹⁰ is absent; and
   k, l and m are each 0; or
wherein:
   T⁷ is absent and V⁷ is —NHCO—;
   T⁸ is (C₁-C₁₂)alkyl and V⁸ is absent;
   T⁹ is heteroaryl and V⁹ is absent;
   T¹⁰ is (C₁-C₁₂)alkyl and V¹⁰ is —CONH—;
   T¹¹ is (PEG)ₙ and V¹¹ is —CO—; and
   l and m are each 0; or
wherein:
   T⁷ is absent and V⁷ is —NHCO—;
   T⁸ is (C₁-C₁₂)alkyl and V⁸ is absent;
   T⁹ is heteroaryl and V⁹ is absent;
   T¹⁰ is (C₁-C₁₂)alkyl and V¹⁰ is —CONH—;
   T¹¹ is substituted (C₁-C₁₂)alkyl and V¹¹ is —CO—;
   T¹² is AA and V¹² is absent; and
   T¹³ PAB and V¹³ is absent; or
wherein:
   T⁷ is absent and V⁷ is —NHCO—;
   T⁸ is (C₁-C₁₂)alkyl and V⁸ is absent;
   T⁹ is heteroaryl and V⁹ is absent;
   T¹⁰ is (C₁-C₁₂)alkyl and V¹⁰ is —CONH—;
   T¹¹ is substituted (C₁-C₁₂)alkyl and V¹¹ is —CO—;
   T¹² is AA and V¹² is absent; and
   T¹³ PABC and V¹³ is absent.

In certain embodiments, the left-hand side of the above linker structure for the second linker L^{B} is attached to the hydrazinyl-indolyl or a hydrazinyl-pyrrolo-pyridinyl conjugation moiety, and the right-hand side of the above linker structure for the second linker L^{B} is attached to the second drug or active agent.

In certain embodiments, the conjugate is an antibody-drug conjugate where the antibody and the drugs are linked together by linkers as described above. In some instances, the linker m (e.g., L^{A} and/or L^{B}) is a cleavable linker. A cleavable linker is a linker that includes one or more cleavable moieties, where the cleavable moiety includes one or more bonds that can dissociate under certain conditions, thus separating the cleavable linker into two or more separable portions. For example, the cleavable moiety may include one or more covalent bonds, which under certain conditions, can dissociate or break apart to separate the cleavable linker into two or more portions. As such the linkers that are included in an antibody-drug conjugate can be cleavable linkers, such that under appropriate conditions, the cleavable linker is cleaved to separate or release the drug from the antibody at a desired target site of action for the drug.

In some instances, a cleavable linker includes two cleavable moieties, such as a first cleavable moiety and a second cleavable moiety. The cleavable moieties can be configured such that cleavage of both cleavable moieties is needed in order to separate or release the drug from the antibody at a desired target site of action for the drug. For example, cleavage of a cleavable linker can be achieved by initially cleaving one of the two cleavable moieties and then cleaving the other of the two cleavable moieties. In certain embodiments, a cleavable linker includes a first cleavable moiety and a second cleavable moiety that hinders cleavage of the first cleavable moiety. By “hinders cleavage” is meant that the presence of an uncleaved second cleavable moiety reduces the likelihood or substantially inhibits the cleavage of the first cleavable moiety, thus substantially reducing the amount or preventing the cleavage of the cleavable linker. For instance, the presence of uncleaved second cleavable moiety can hinder cleavage of the first cleavable moiety. The hinderance of cleavage of the first cleavable moiety by the presence of the second cleavable moiety, in turn, substantially reduces the amount or prevents the release of the drug from the antibody. For example, the premature release of the drug from the antibody can be substantially reduced or prevented until the antibody-drug conjugate is at or near the desired target site of action for the drug.

In some cases, since the second cleavable moiety hinders cleavage of the first cleavable moiety, cleavage of the cleavable linker can be achieved by initially cleaving the second cleavable moiety and then cleaving the first cleavable moiety. Cleavage of the second cleavable moiety can reduce or eliminate the hinderance on the cleavage of the first cleavable moiety, thus allowing the first cleavable moiety to be cleaved. Cleavage of the first cleavable moiety can result in the cleavable linker dissociating or separating into two or more portions as described above to release the drug from the antibody-drug conjugate. In some instances, cleavage of the first cleavable moiety does not substantially occur in the presence of an uncleaved second cleavable moiety. By substantially is meant that about 10% or less cleavage of the first cleavable moiety occurs in the presence of an uncleaved second cleavable moiety, such as about 9% or less, or about 8% or less, or about 7% or less, or about 6% or less, or about 5% or less, or about 4% or less, or about 3% or less, or about 2% or less, or about 1% or less, or about 0.5% or less, or about 0.1% or less cleavage of the first cleavable moiety occurs in the presence of an uncleaved second cleavable moiety.

Stated another way, the second cleavable moiety can protect the first cleavable moiety from cleavage. For instance, the presence of uncleaved second cleavable moiety can protect the first cleavable moiety from cleavage, and thus substantially reduce or prevent premature release of the drug from the antibody until the antibody-drug conjugate is at or near the desired target site of action for the drug. As such, cleavage of the second cleavable moiety exposes the first cleavable moiety (e.g., deprotects the first cleavable moiety), thus allowing the first cleavable moiety to be cleaved, which results in cleavage of the cleavable linker, which, in turn, separates or releases the drug from the antibody at a desired target site of action for the drug as described above. In certain instances, cleavage of the second cleavable moiety exposes the first cleavable moiety to subsequent cleavage, but cleavage of the second cleavable moiety does not in and of itself result in cleavage of the cleavable linker (i.e., cleavage of the first cleavable moiety is still needed in order to cleave the cleavable linker).

The cleavable moieties included in the cleavable linker may each be an enzymatically cleavable moiety. For example, the first cleavable moiety can be a first enzymatically cleavable moiety and the second cleavable moiety can be a second enzymatically cleavable moiety. An enzymatically cleavable moiety is a cleavable moiety that can be separated into two or more portions as described above through the enzymatic action of an enzyme. The enzymatically cleavable moiety can be any cleavable moiety that can be cleaved through the enzymatic action of an enzyme, such as, but not limited to, an ester, a peptide, a glycoside, and the like. In some instances, the enzyme that cleaves the enzymatically cleavable moiety is present at a desired target site of action, such as the desired target site of action of the drug that is to be released from the antibody-drug conjugate. In some cases, the enzyme that cleaves the enzymatically cleavable moiety is not present in a significant amount in other areas, such as in whole blood, plasma or serum. As such, the cleavage of an enzymatically cleavable moiety can be controlled such that substantial cleavage occurs at the desired site of action, whereas cleavage does not significantly occur in other areas or before the antibody-drug conjugate reaches the desired site of action.

For example, as described herein, antibody-drug conjugates of the present disclosure can be used for the treatment of cancer, such as for the delivery of a cancer therapeutic drug to a desired site of action where the cancer cells are present. In some cases, enzymes, such as an esterase that cleaves ester bonds or a glycosidase that cleaves glycosidic bonds, can be a biomarker for cancer that is overexpressed in cancer cells. The overexpression, and thus localization, of certain enzymes in cancer can be used in the context of the enzymatically cleavable moieties included in the cleavable linkers of the antibody-drug conjugates of the present disclosure to specifically release the drug at the desired site of action (i.e., the site of the cancer (and overexpressed enzyme)). Thus, in some embodiments, the enzymatically cleavable moiety is a cleavable moiety (e.g., an ester or a glycoside) that can be cleaved by an enzyme that is overexpressed in cancer cells. For instance, the enzyme can be an esterase. As such, in some instances, the enzymatically cleavable moiety is a cleavable moiety (e.g., an ester) that can be cleaved by an esterase enzyme. In some instances, the enzyme can be a glycosidase. As such, in some instances, the enzymatically cleavable moiety is a cleavable moiety (e.g., a glycoside or glycoside derivative) that can be cleaved by a glycosidase enzyme.

In certain embodiments, the enzymatically cleavable moiety is an ester bond. For example, the first cleavable moiety described above (i.e., the cleavable moiety protected from premature cleavage by the second cleavable moiety) can include an ester. The presence of uncleaved second cleavable moiety can protect the first cleavable moiety (ester) from cleavage by an esterase enzyme, and thus substantially reduce or prevent premature release of the drug from the antibody until the antibody-drug conjugate is at or near the desired target site of action for the drug. In some instances, a portion of the linker adjacent to the first cleavable moiety is linked to or includes a substituent, where the substituent comprises the second cleavable moiety. In some instances, the second cleavable moiety includes a glycoside or glycoside derivative.

In some embodiments, the enzymatically cleavable moiety is sugar moiety, such as a glycoside (or glyosyl) or glycoside derivative. In some cases, the glycoside or glycoside derivative can facilitate an increase in the hydrophilicity of the cleavable linker as compared to a cleavable linker that does not include the glycoside or glycoside derivative. The glycoside or glycoside derivative can be any glycoside or glycoside derivative suitable for use in the cleavable linker and that can be cleaved through the enzymatic action of an enzyme. For example, the second cleavable moiety (i.e., the cleavable moiety that protects the first cleavable moiety from premature cleavage) can be a glycoside or glycoside derivative. For instance, in some embodiments, the first cleavable moiety includes an ester and the second cleavable moiety includes a glycoside or glycoside derivative. In certain embodiments, the second cleavable moiety is a glycoside or glycoside derivative selected from a glucuronide, a galactoside, a glucoside, a mannoside, a fucoside, O-GlcNAc, and O-GalNAc. In some instances, the second cleavable moiety is a glucuronide. In some instances, the second cleavable moiety is a galactoside. In some instances, the second cleavable moiety is a glucoside. In some instances, the second cleavable moiety is a mannoside. In some instances, the second cleavable moiety is a fucoside. In some instances, the second cleavable moiety is O-GlcNAc. In some instances, the second cleavable moiety is O-GalNAc.

The glycoside or glycoside derivative can be attached (covalently bonded) to the cleavable linker through a glycosidic bond. The glycosidic bond can link the glycoside or glycoside derivative to the cleavable linker through various types of bonds, such as, but not limited to, an O-glycosidic bond (an O-glycoside), an N-glycosidic bond (a glycosylamine), an S-glycosidic bond (a thioglycoside), or C-glycosidic bond (a C-glycoside or C-glycosyl). In some instances, the glycosidic bond is an O-glycosidic bond (an O-glycoside). In some cases, the glycoside or glycoside derivative can be cleaved from the cleavable linker it is attached to by an enzyme (e.g., through enzymatically-mediated hydrolysis of the glycosidic bond). A glycoside or glycoside derivative can be removed or cleaved from the cleavable linker by any convenient enzyme that is able to carry out the cleavage (hydrolysis) of the glycosidic bond that attaches the glycoside or glycoside derivative to the cleavable linker. An example of an enzyme that can be used to mediate the cleavage (hydrolysis) of the glycosidic bond that attaches the glycoside or glycoside derivative to the cleavable linker is a glycosidase, such as a glucuronidase, a galactosidase, a glucosidase, a mannosidase, a fucosidase, and the like. Other suitable enzymes may also be used to mediate the cleavage (hydrolysis) of the glycosidic bond that attaches the glycoside or glycoside derivative to the cleavable linker. In some cases, the enzyme used to mediate the cleavage (hydrolysis) of the glycosidic bond that attaches the glycoside or glycoside derivative to the cleavable linker is found at or near the desired site of action for the drug of the antibody-drug conjugate. For instance, the enzyme can be a lysosomal enzyme, such as a lysosomal glycosidase, found in cells at or near the desired site of action for the drug of the antibody-drug conjugate. In some cases, the enzyme is an enzyme found at or near the target site where the enzyme that mediates cleavage of the first cleavable moiety is found.

Examples of conjugates according to the present disclosure include, but are not limited to, the following structures:

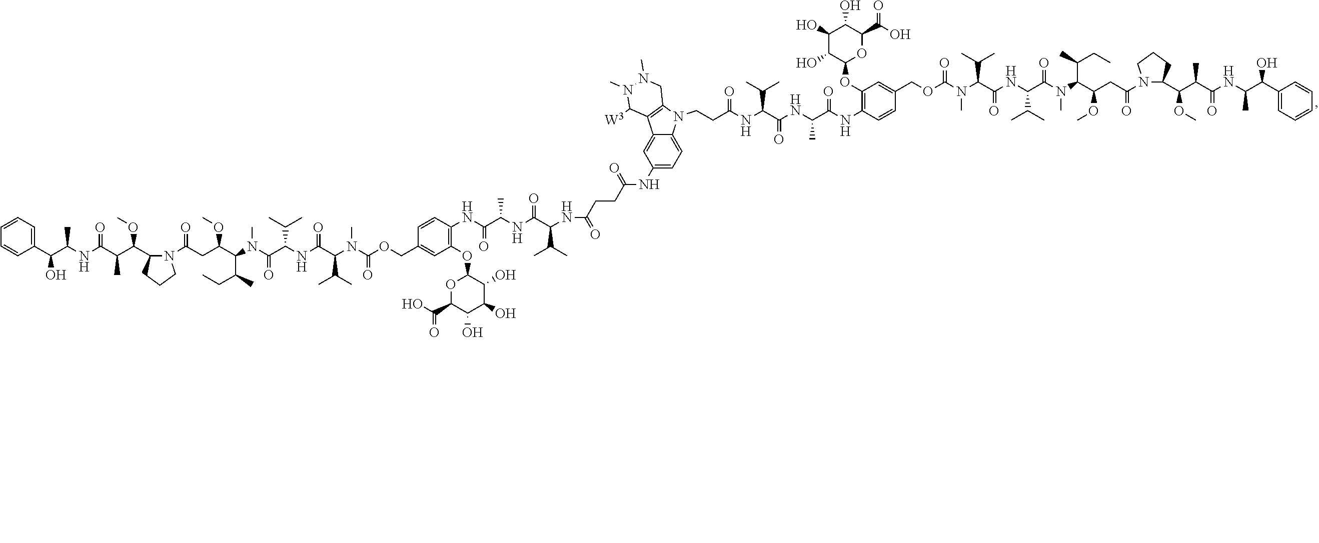

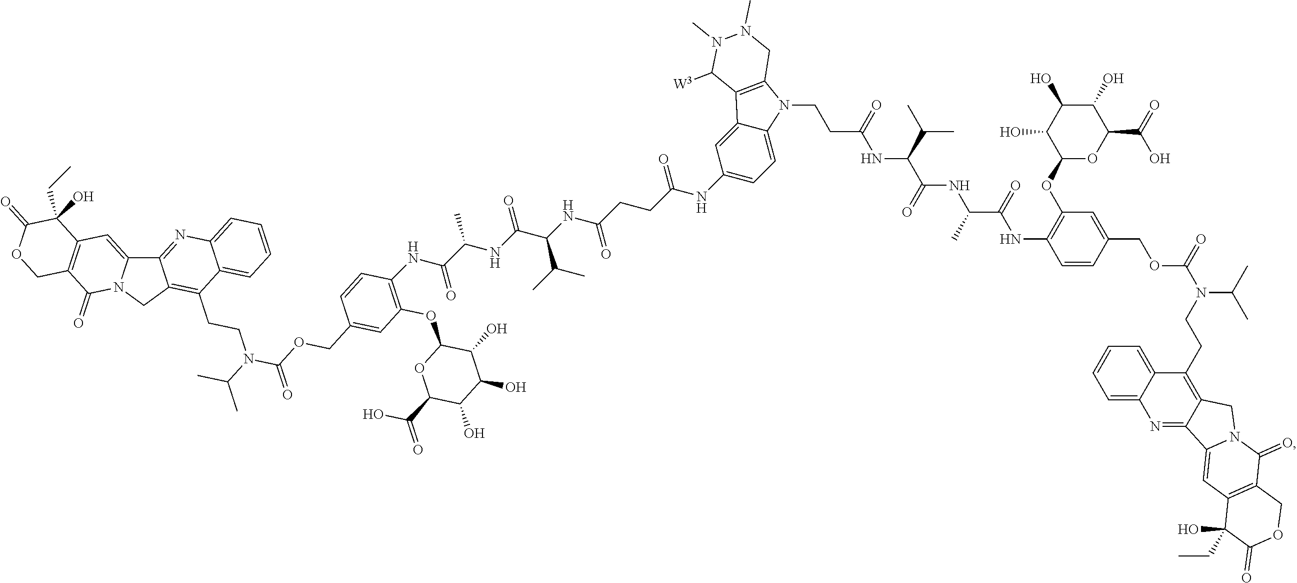

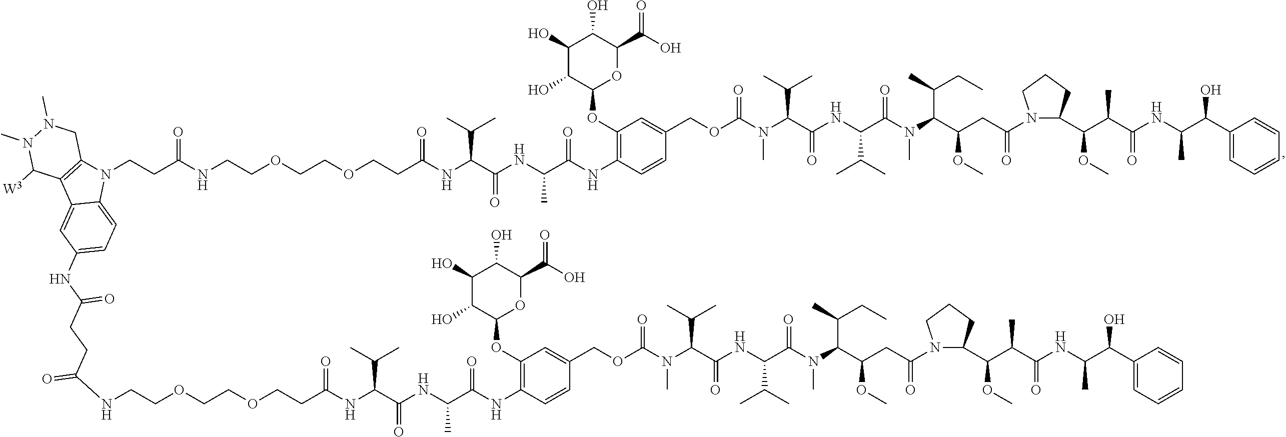

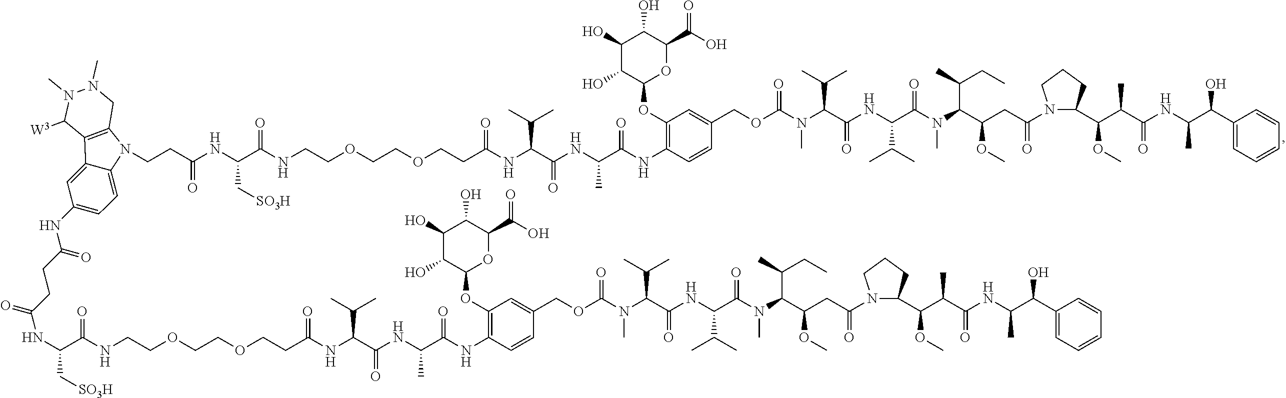

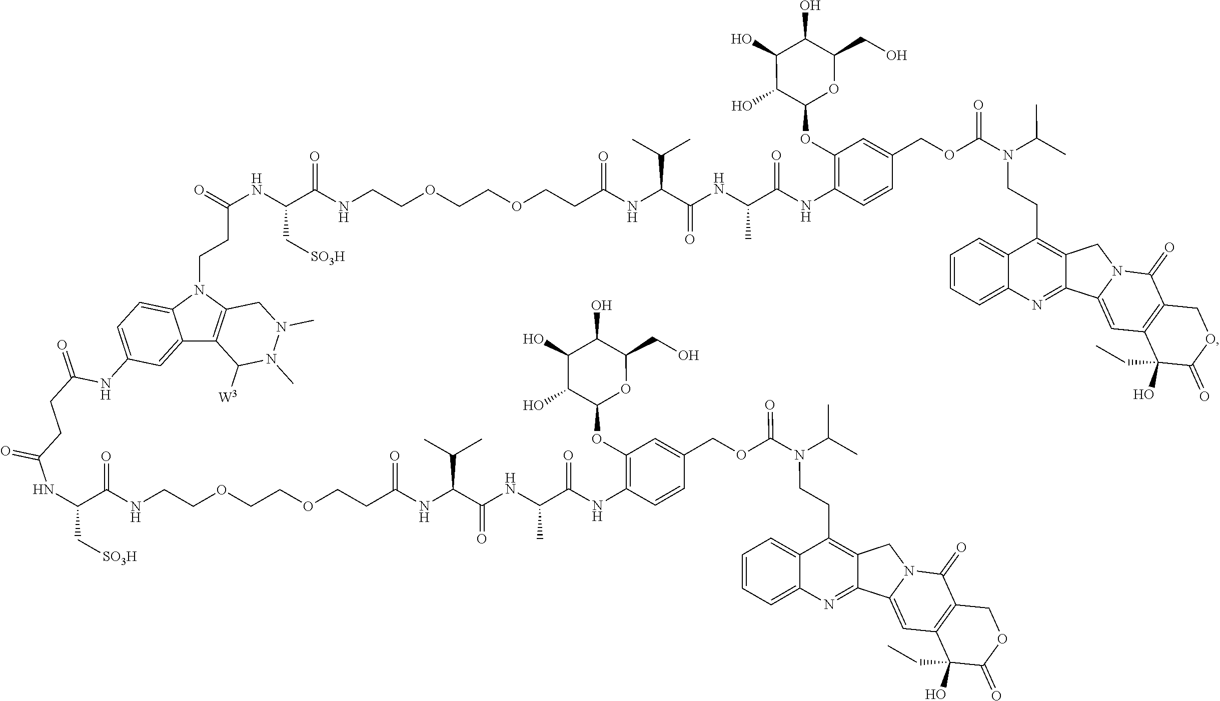

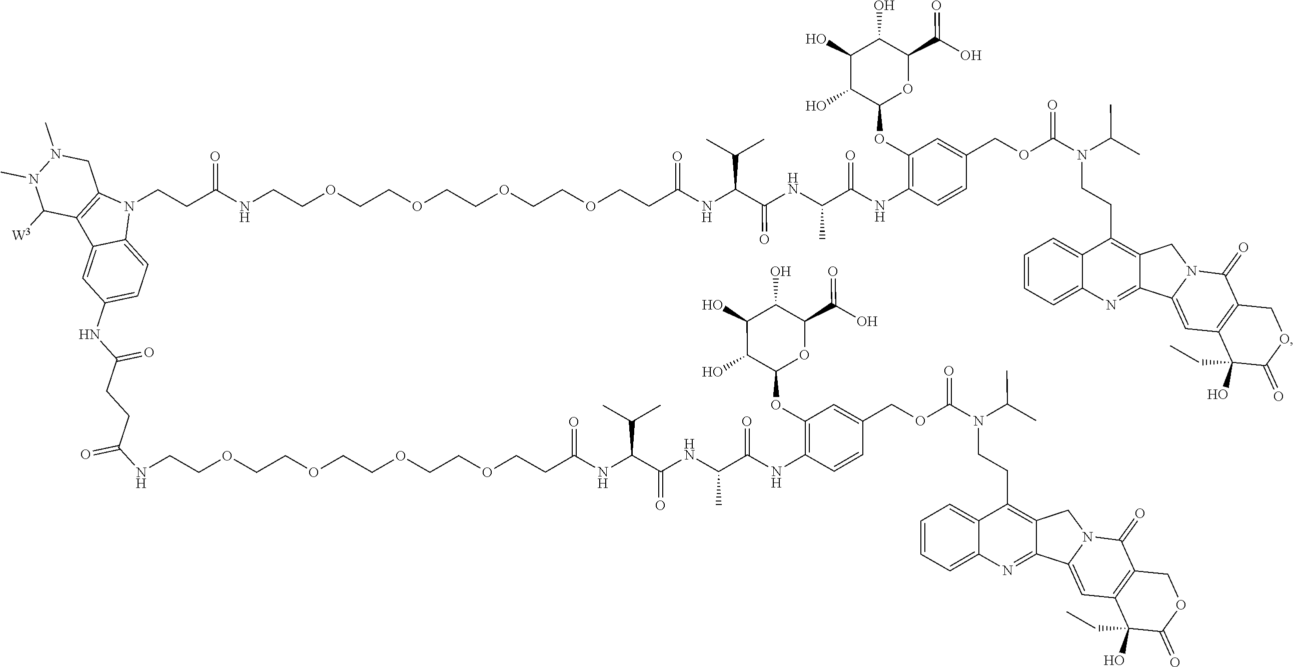

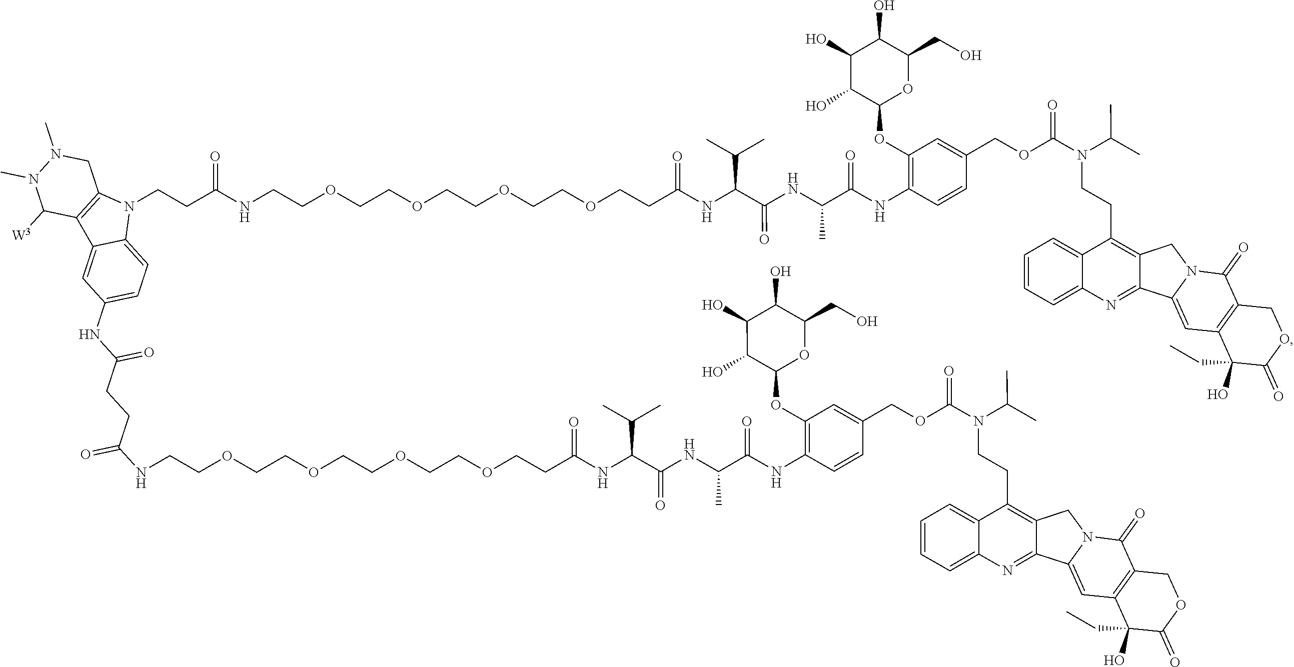

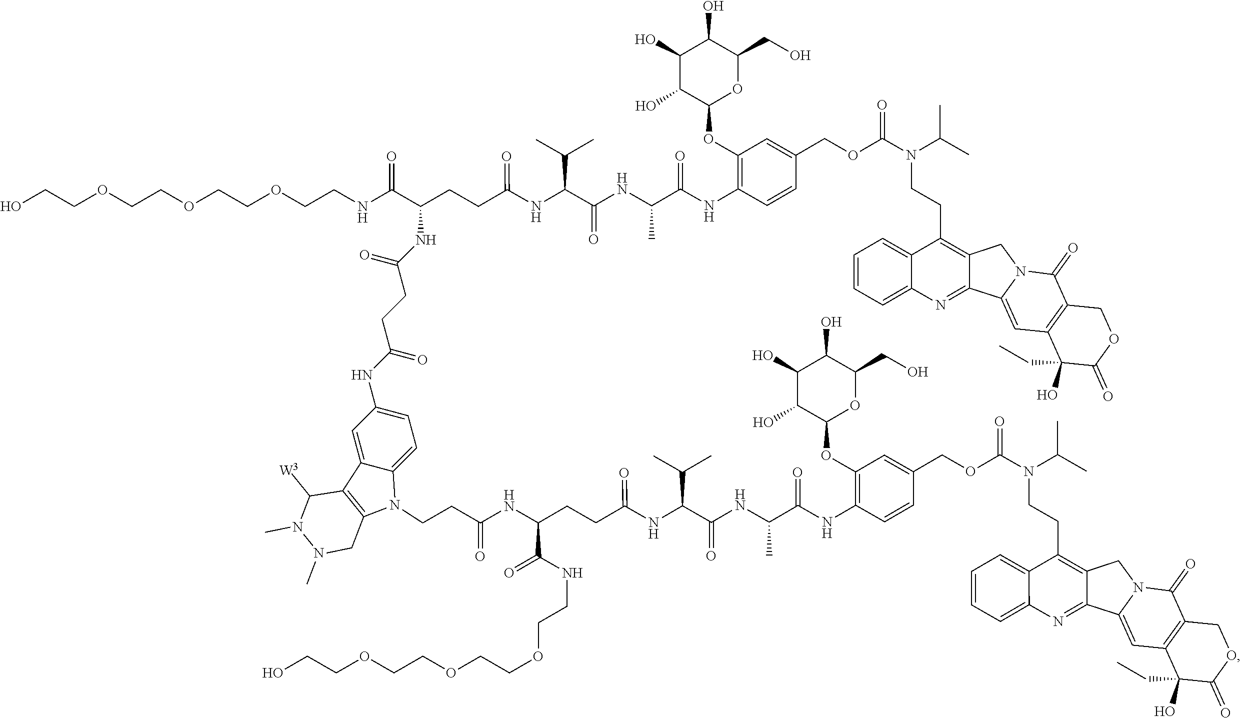

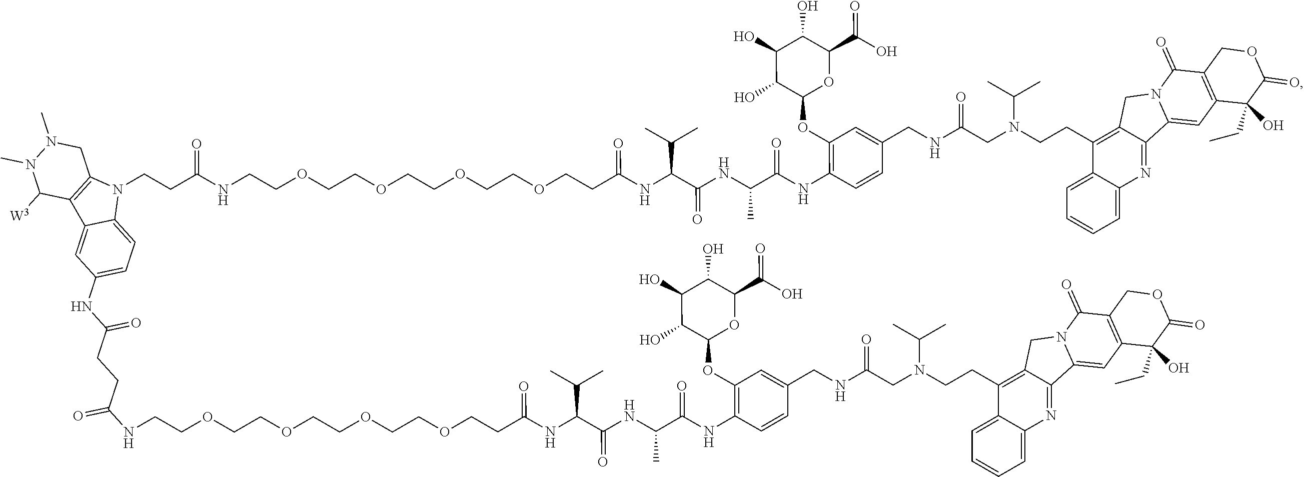

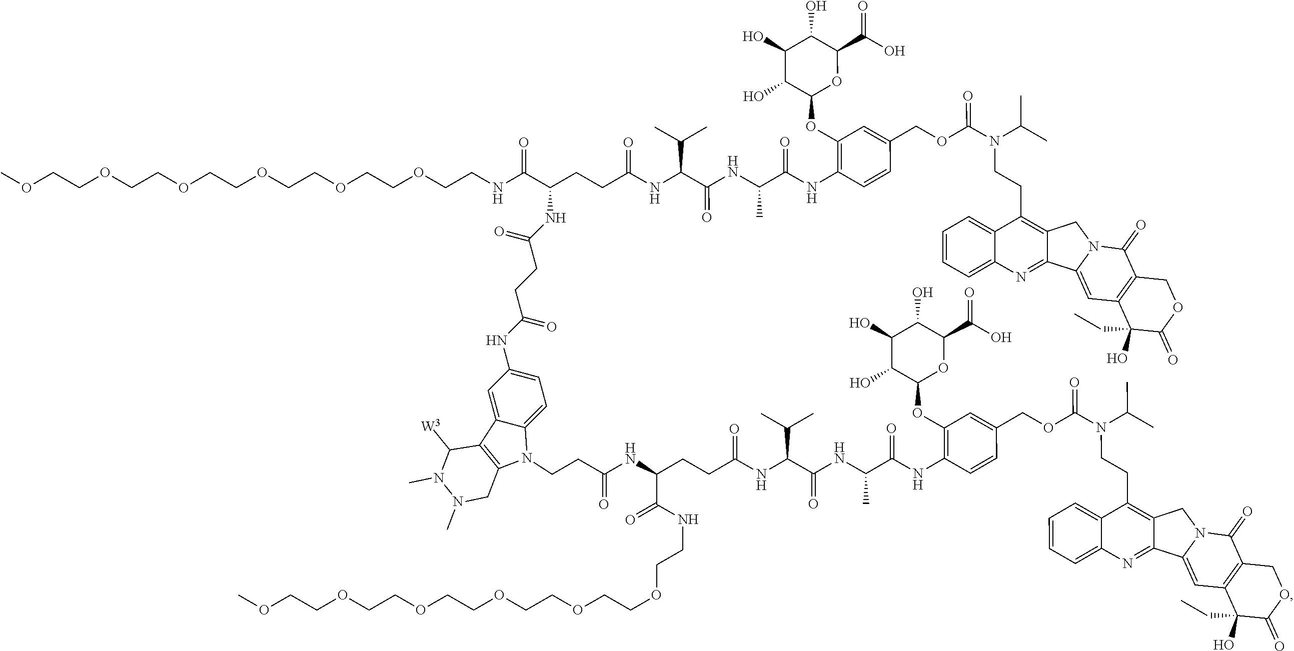

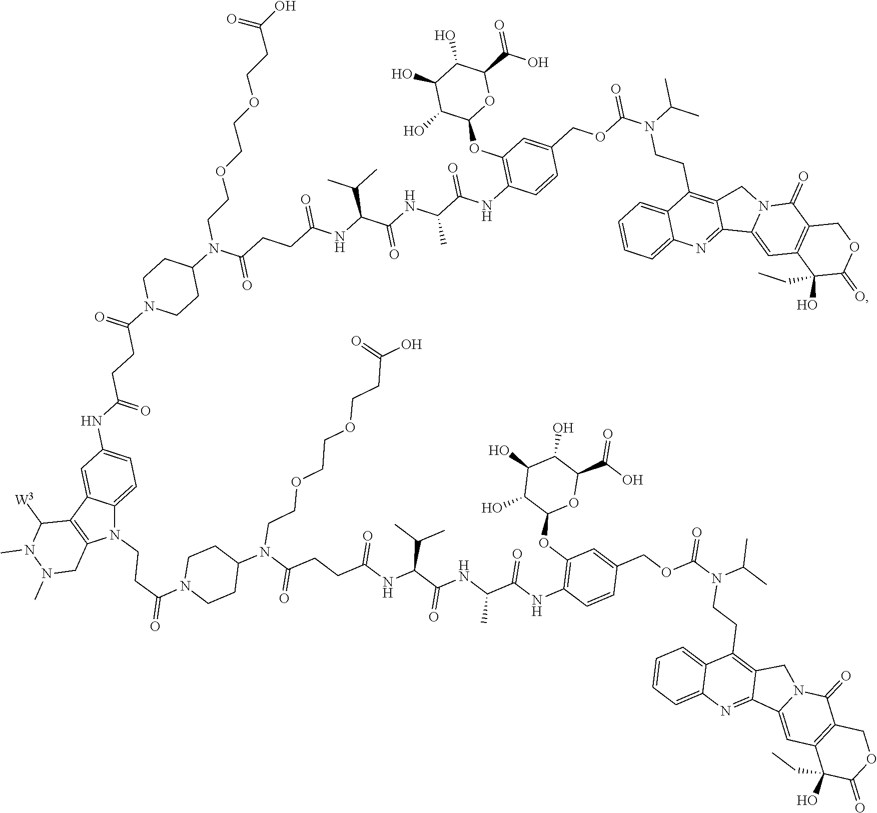

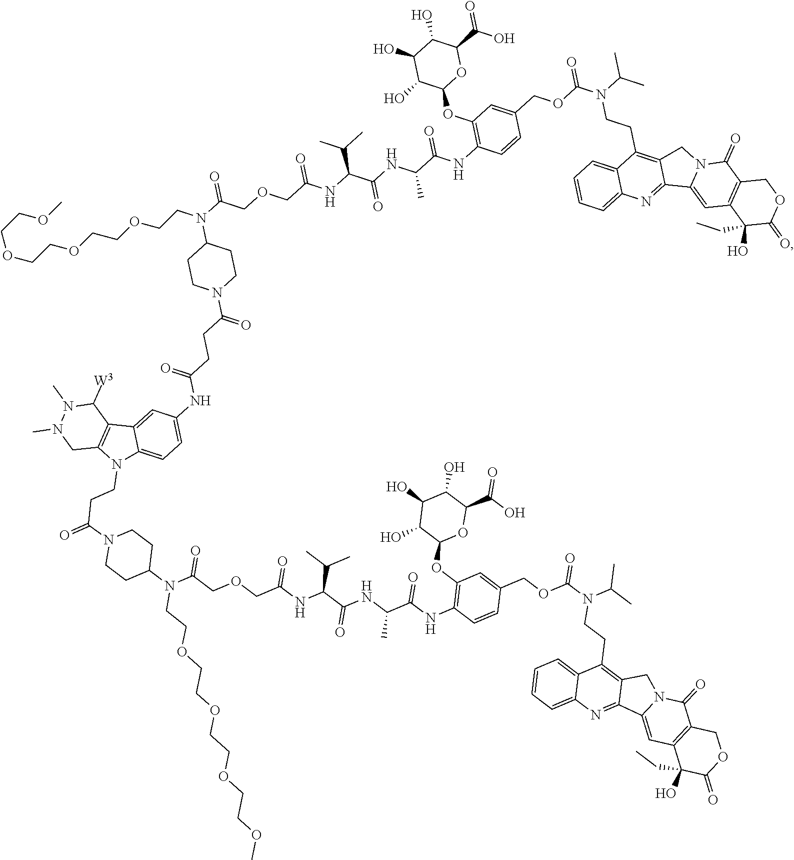

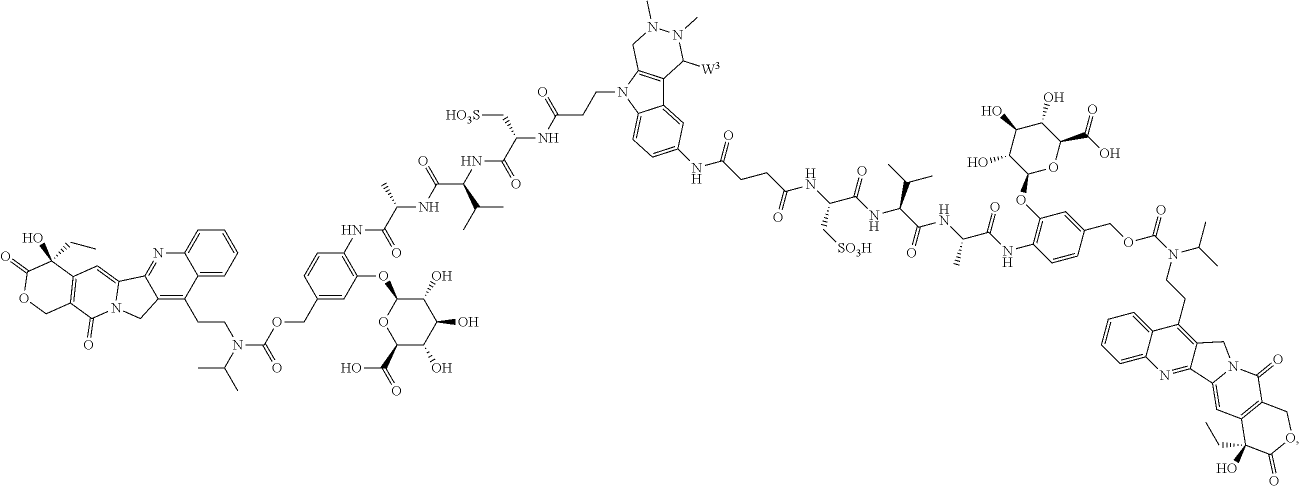

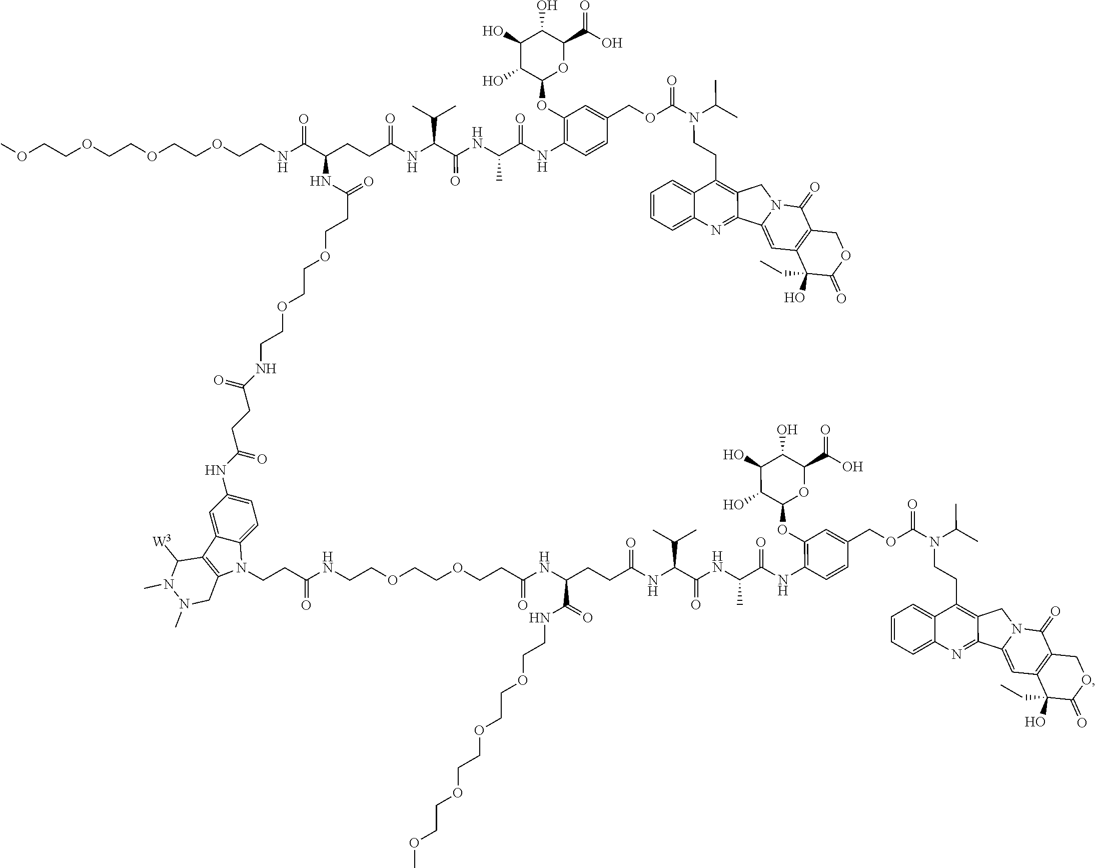

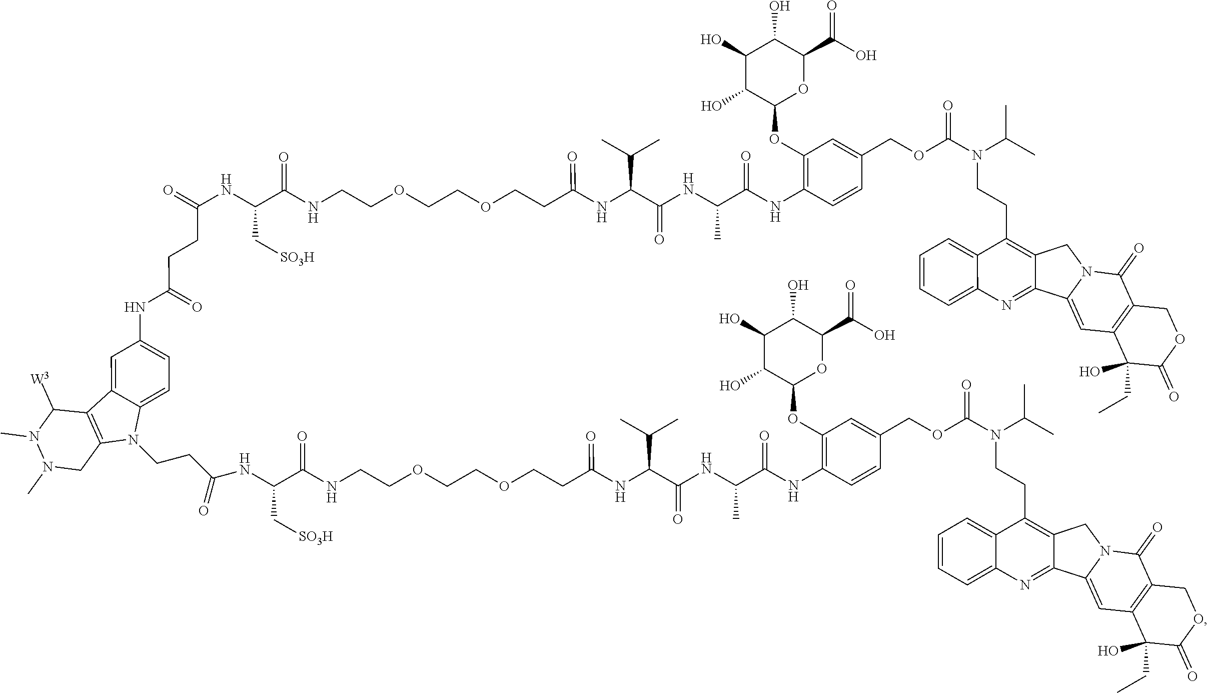

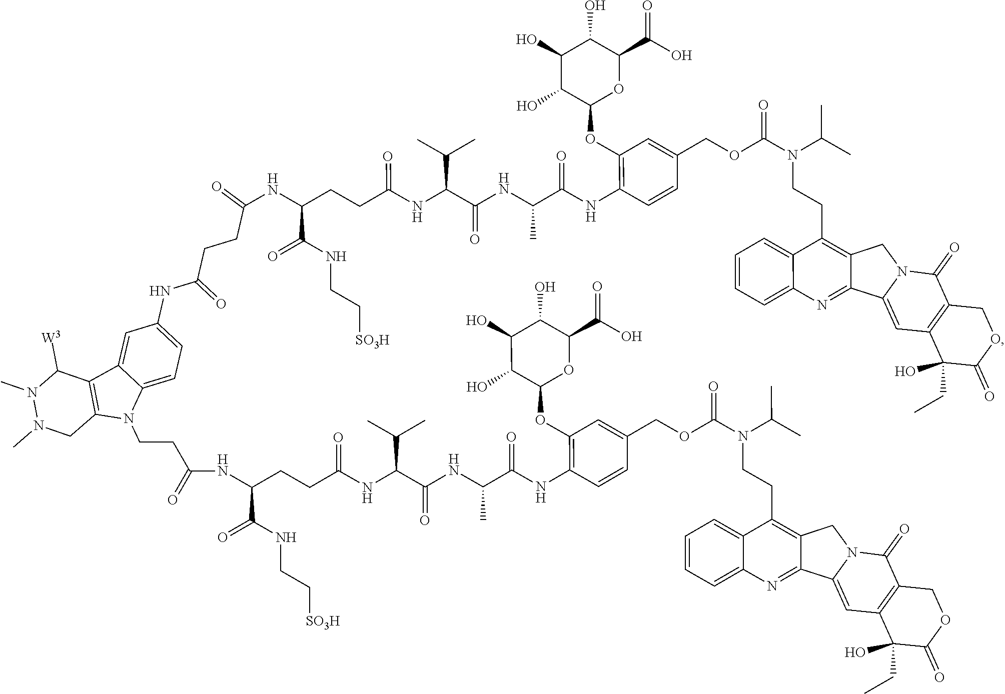

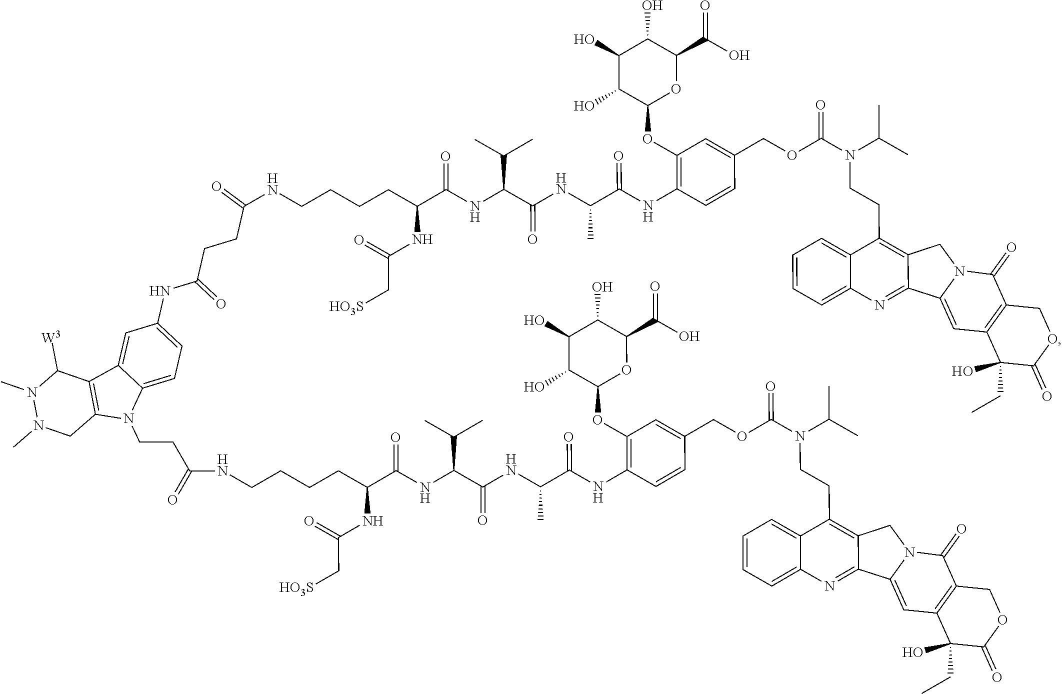

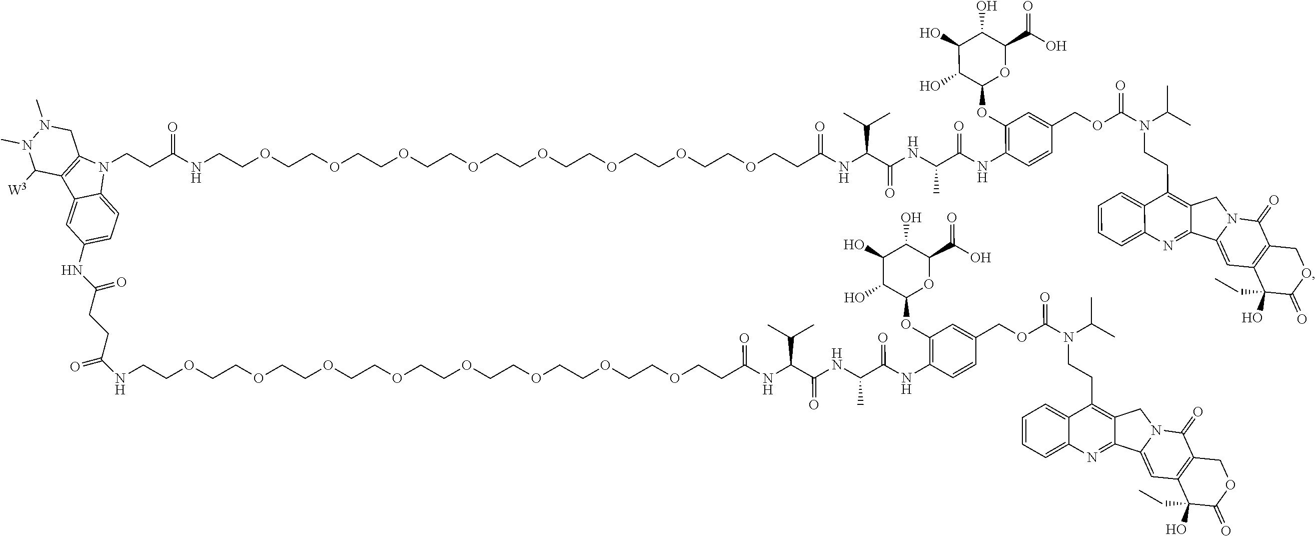

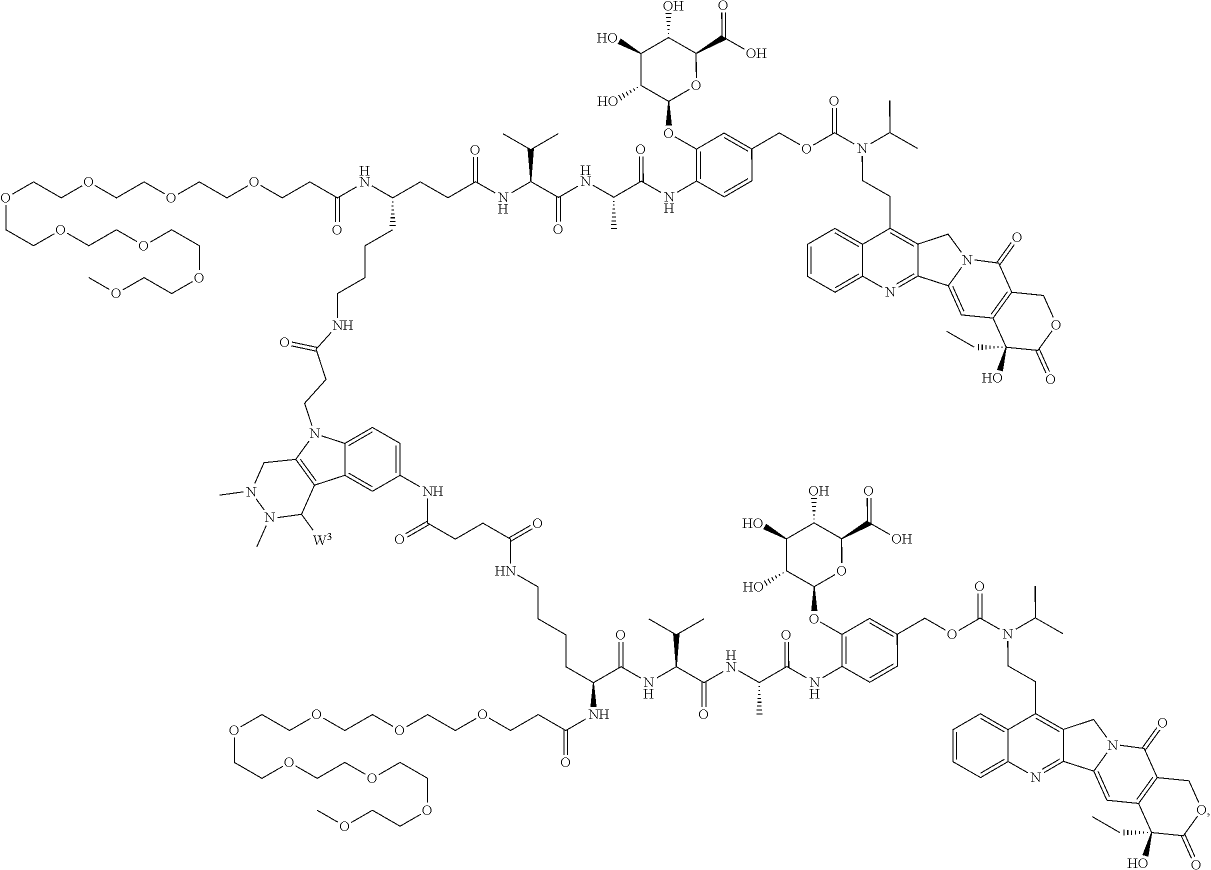

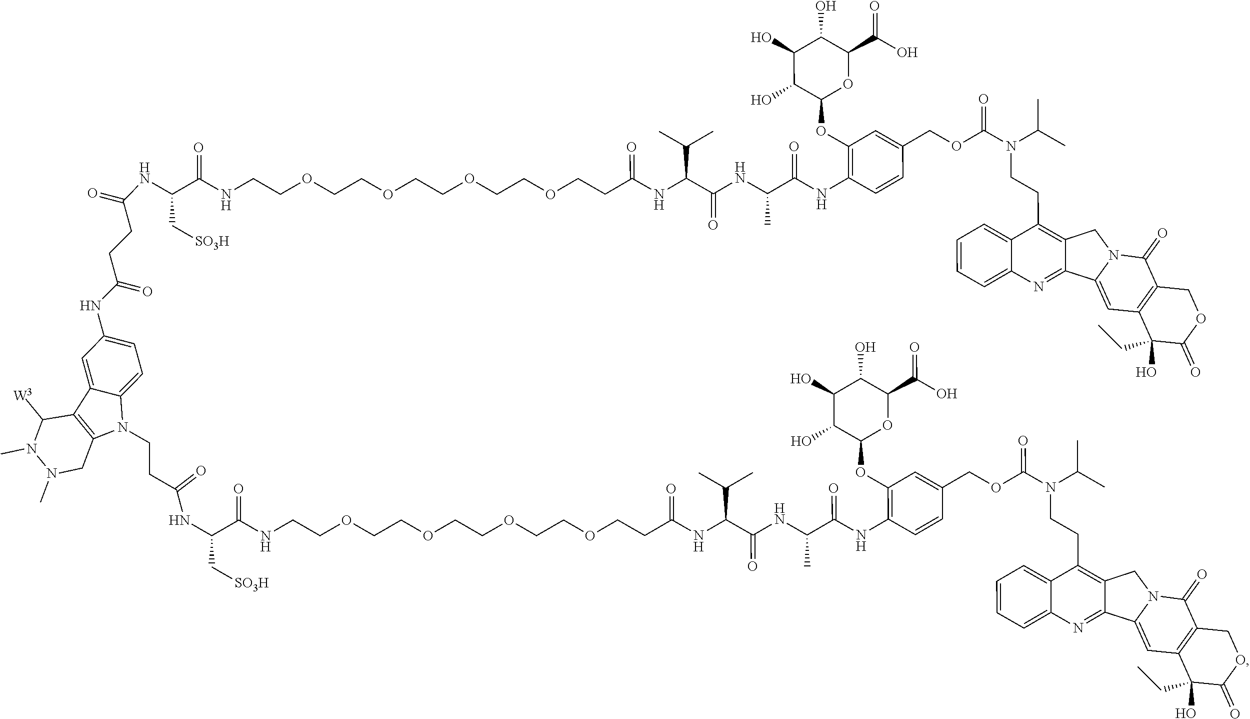

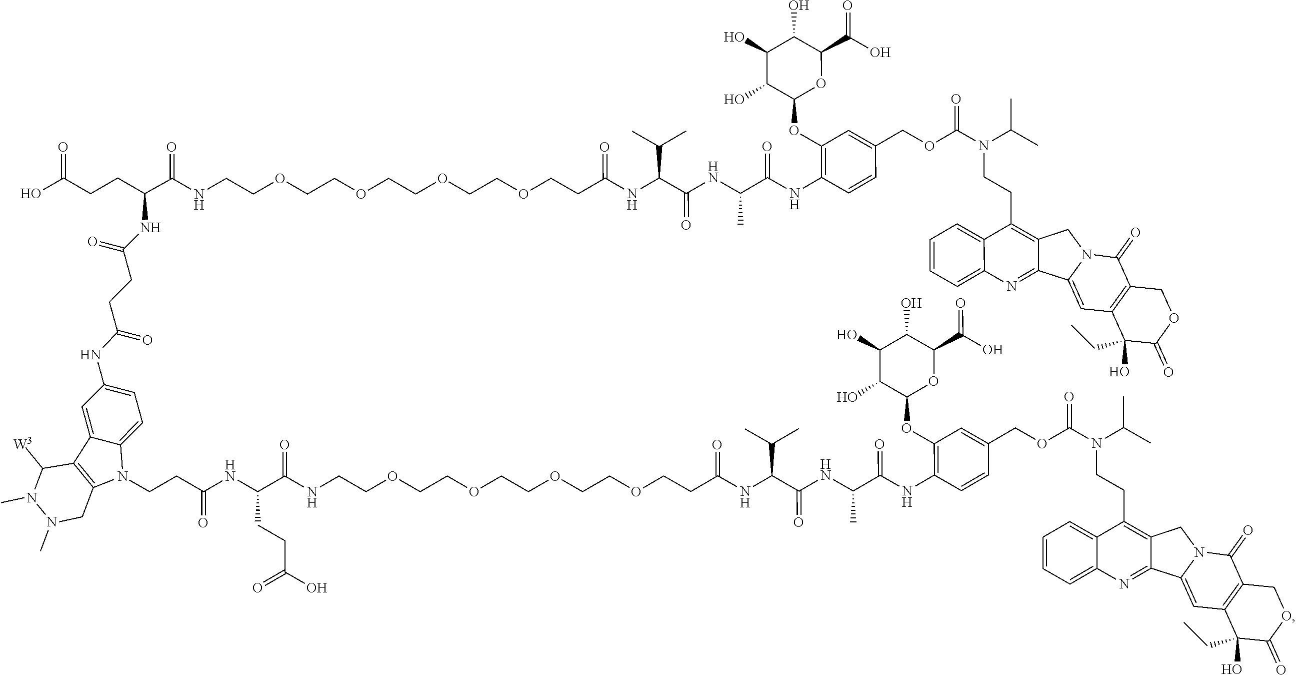

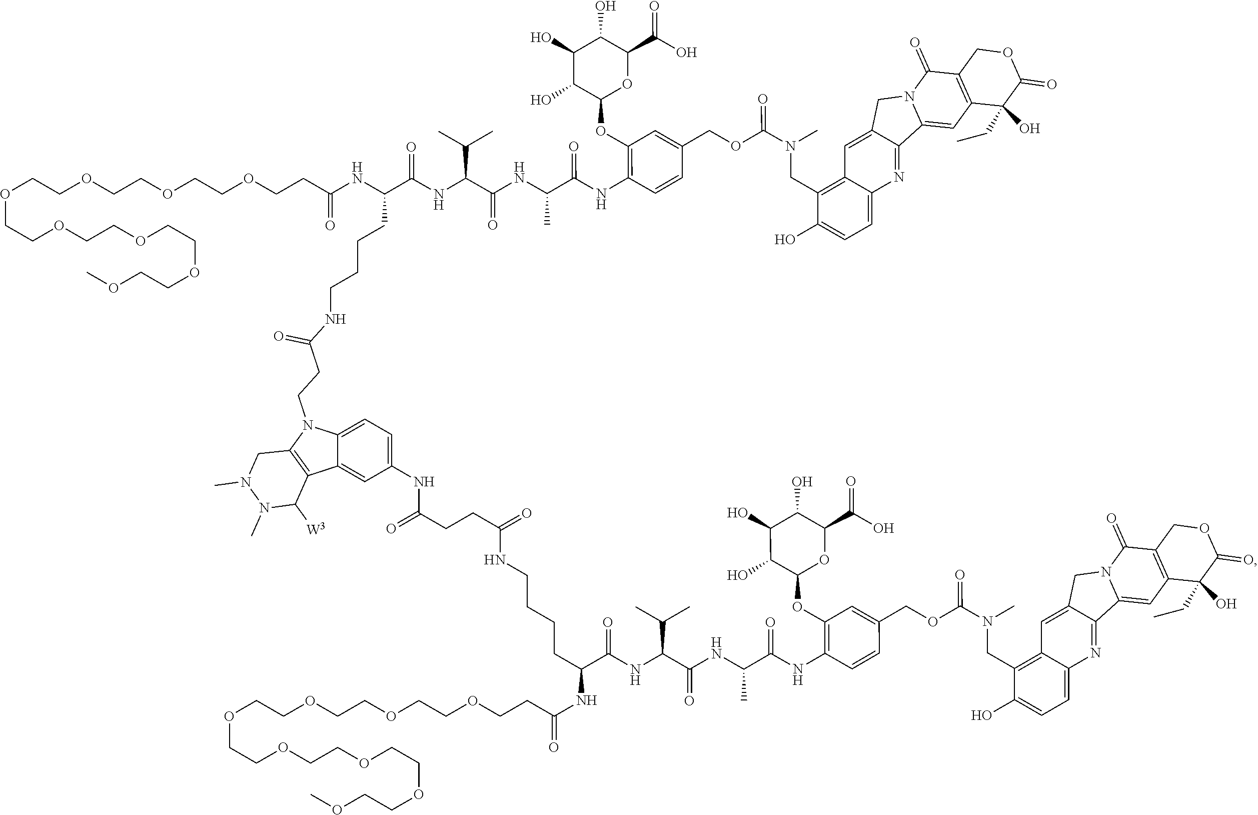

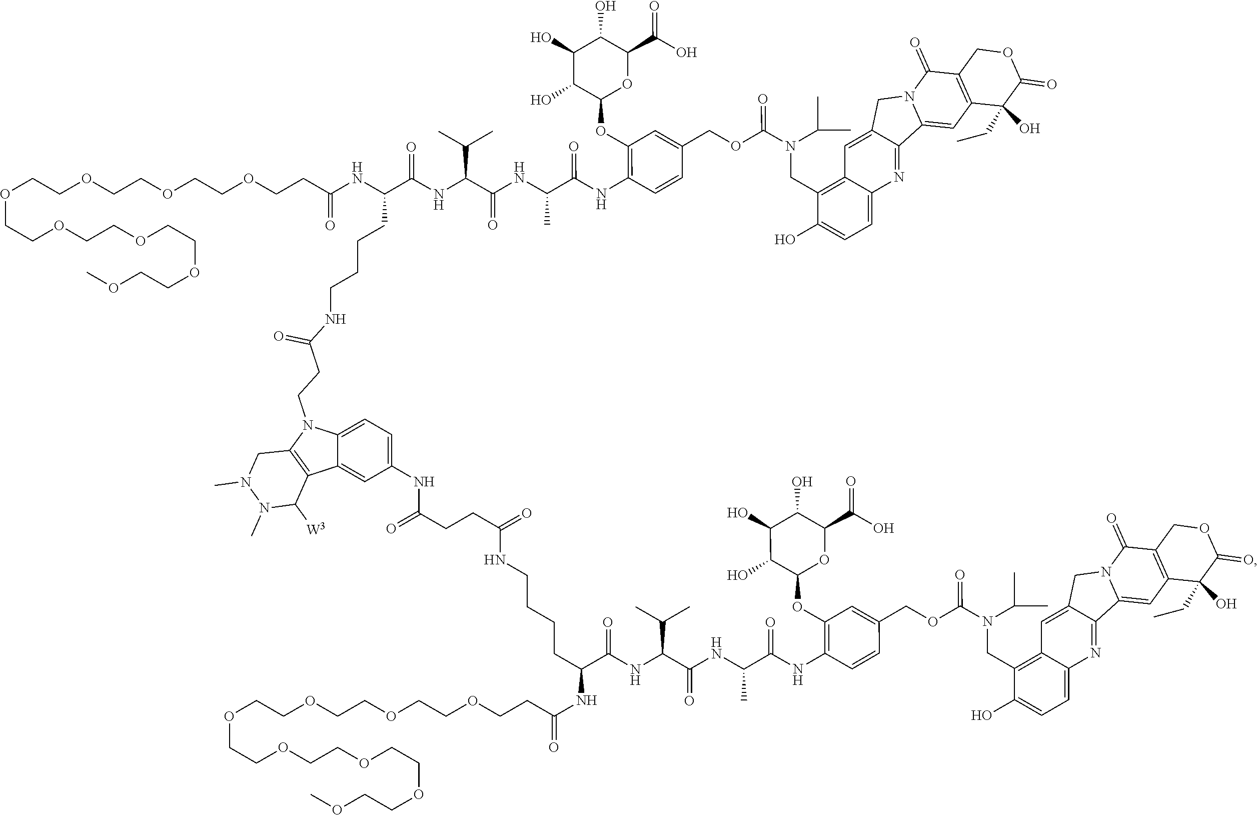

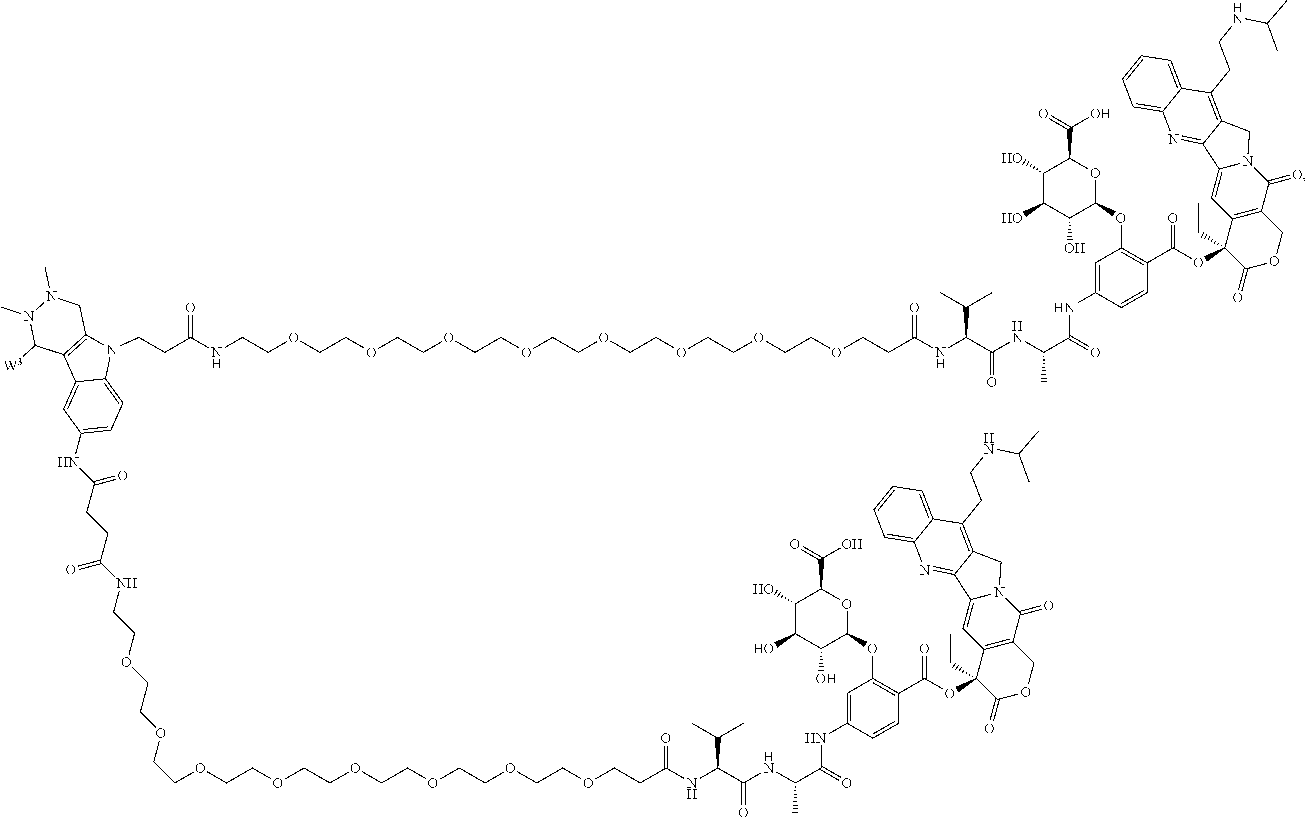

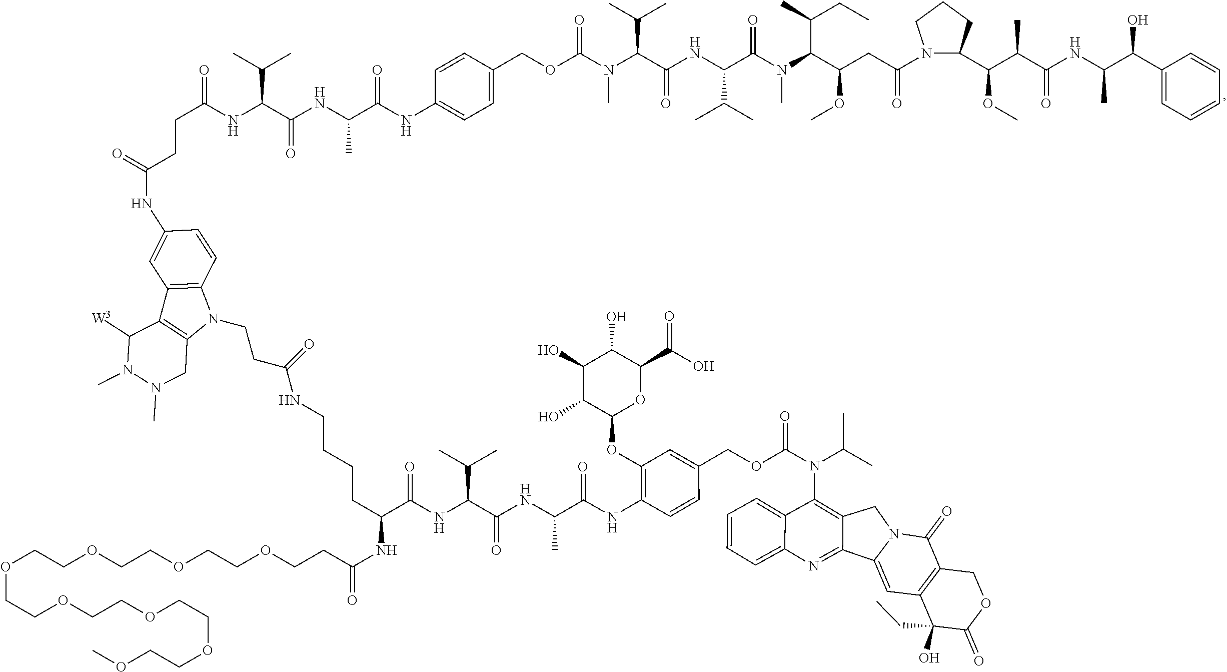

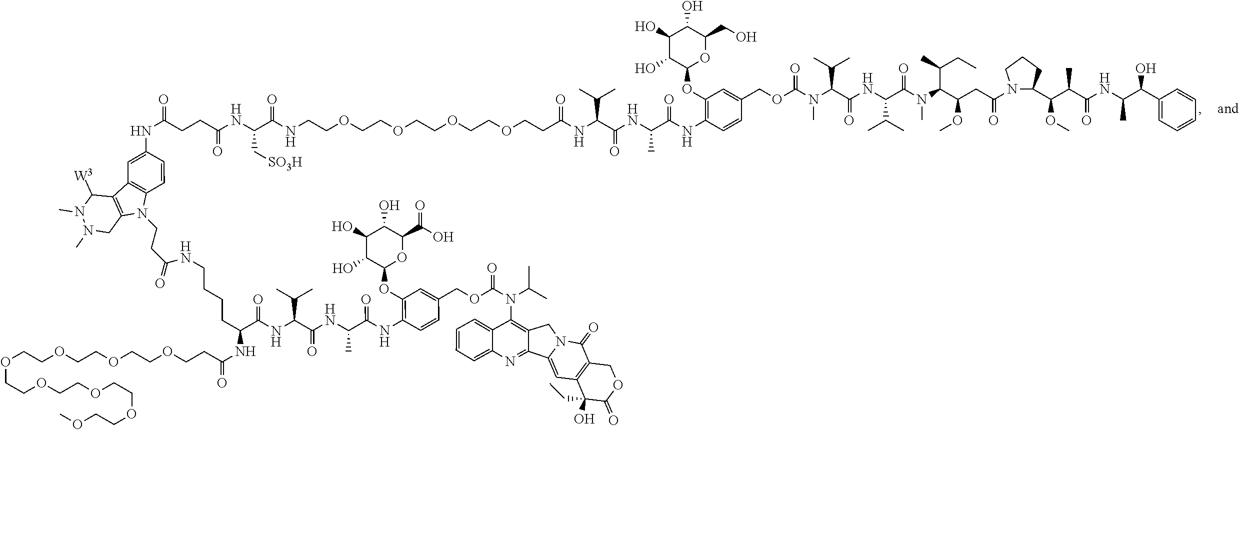

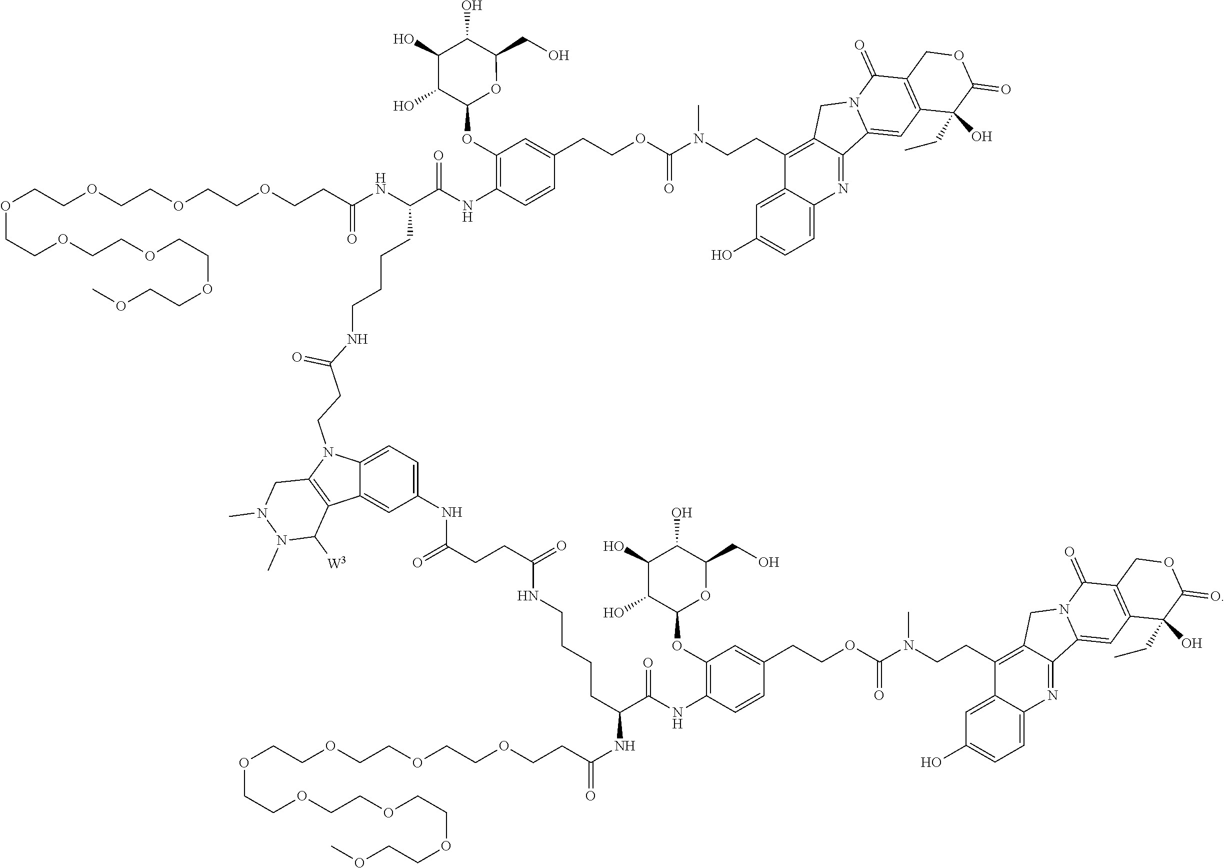

Examples of conjugates according to the present disclosure include, but are not limited to, the following structures:

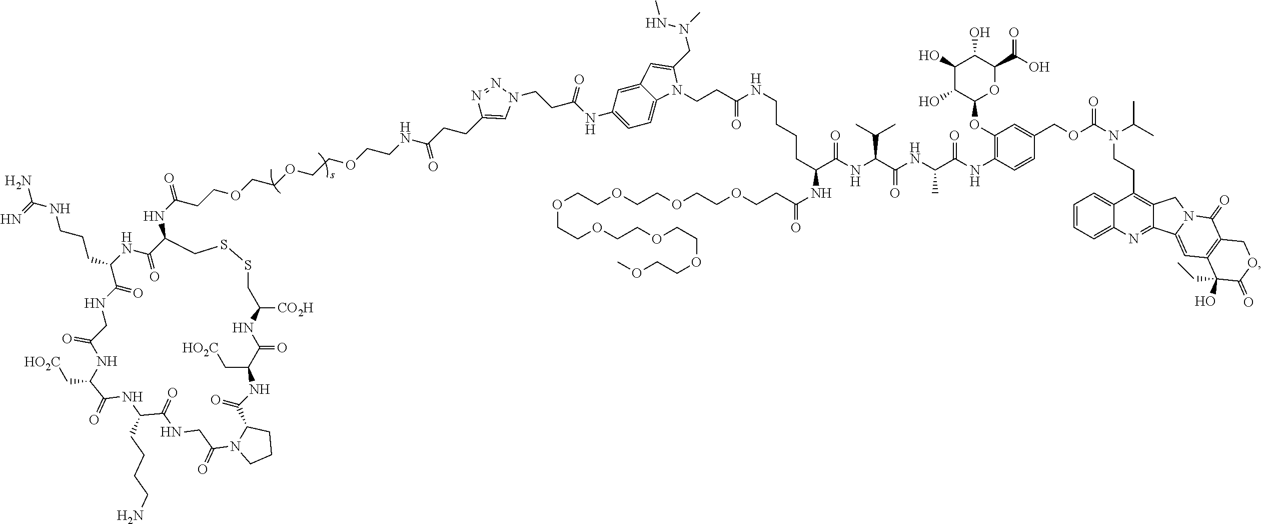

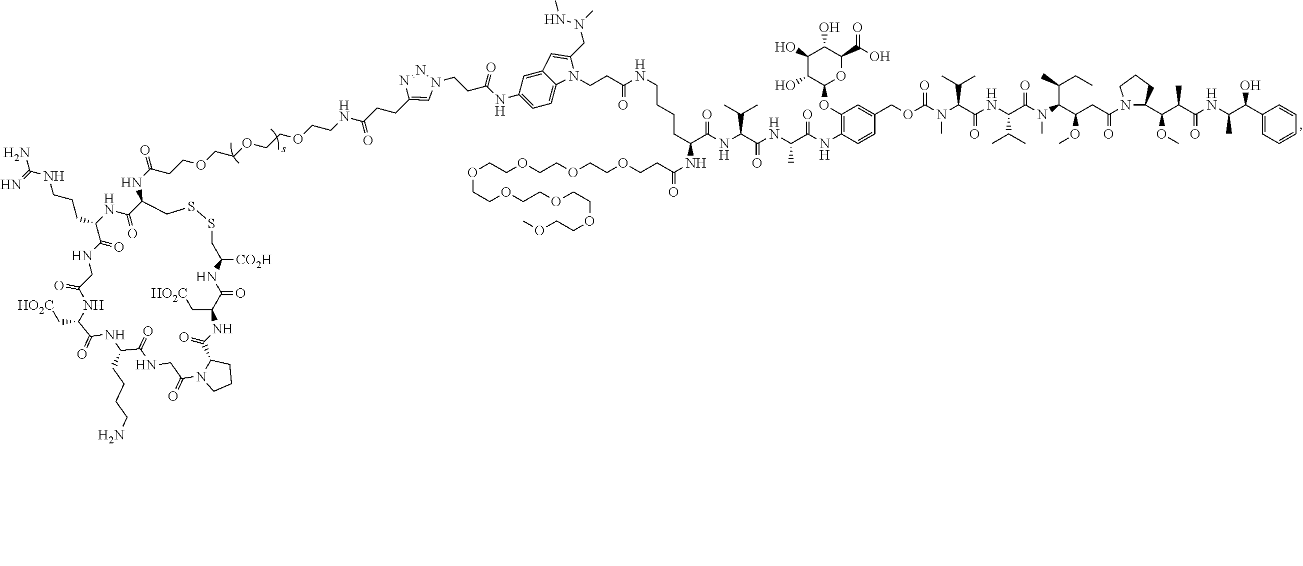

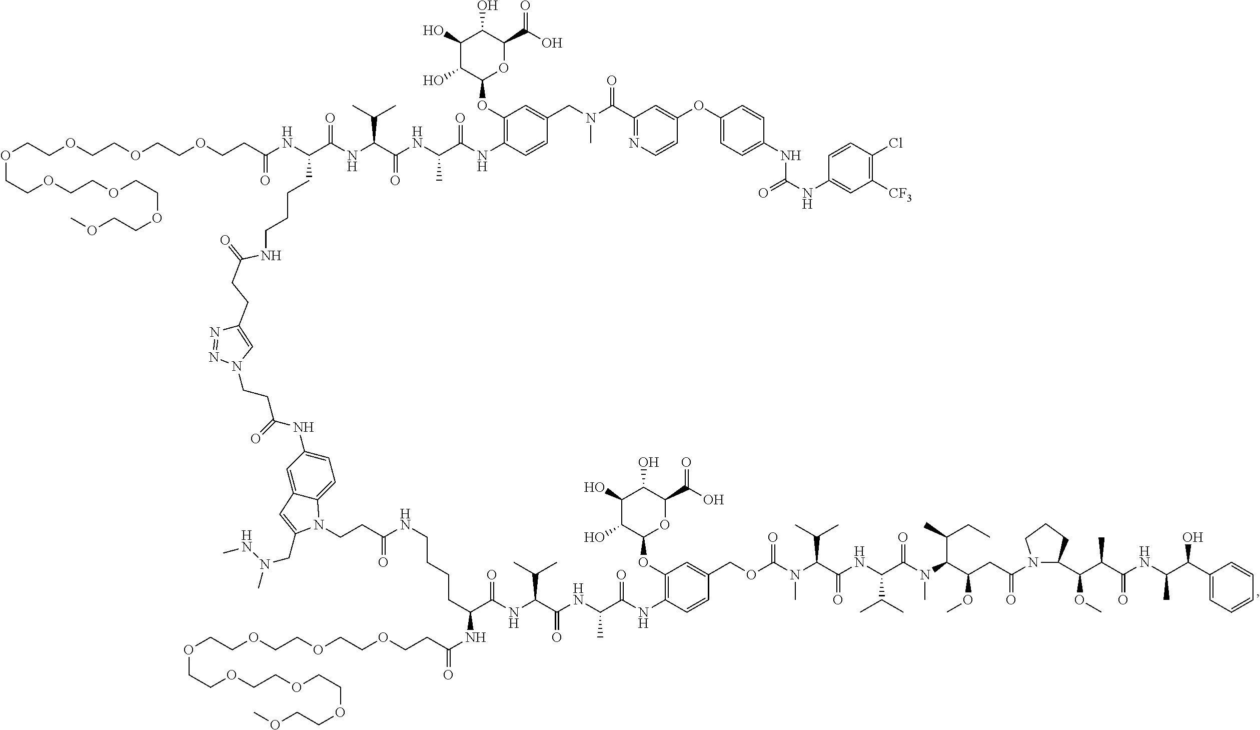

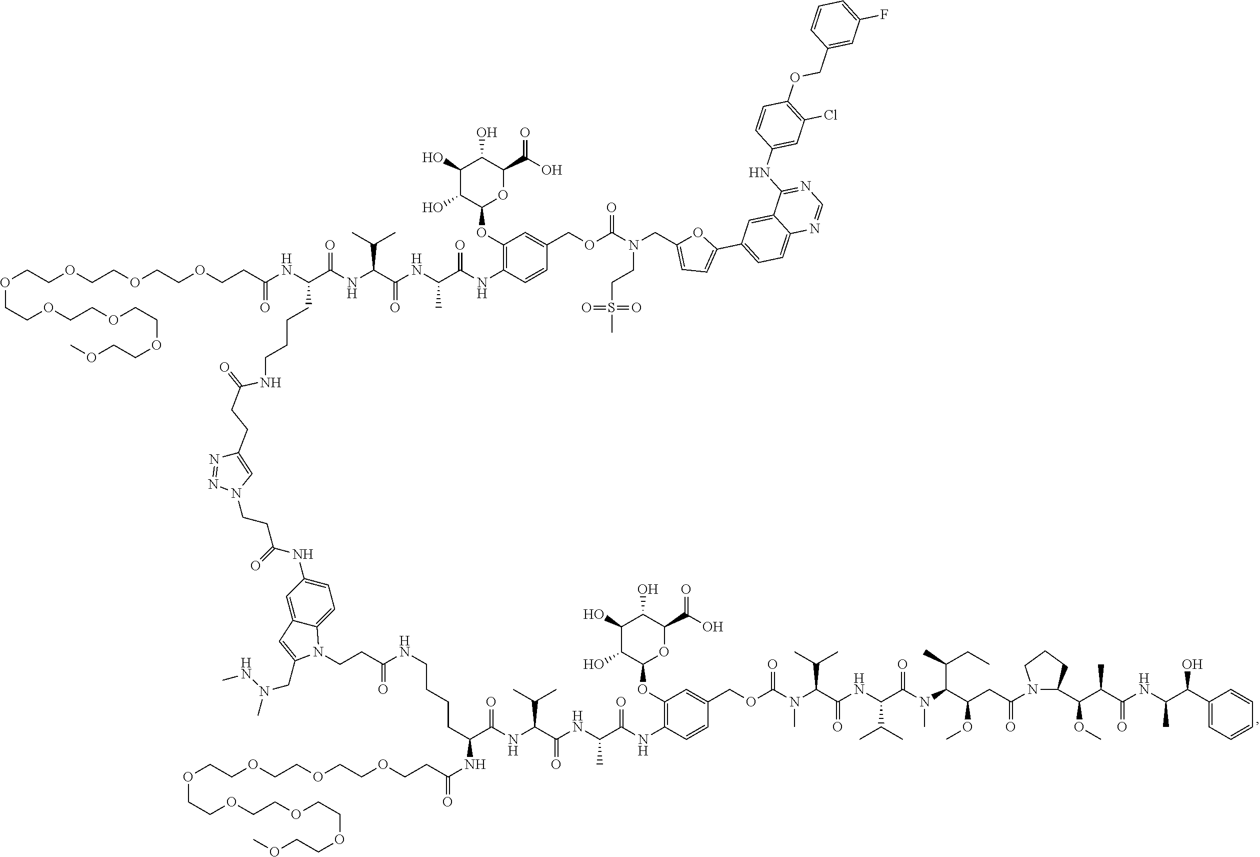

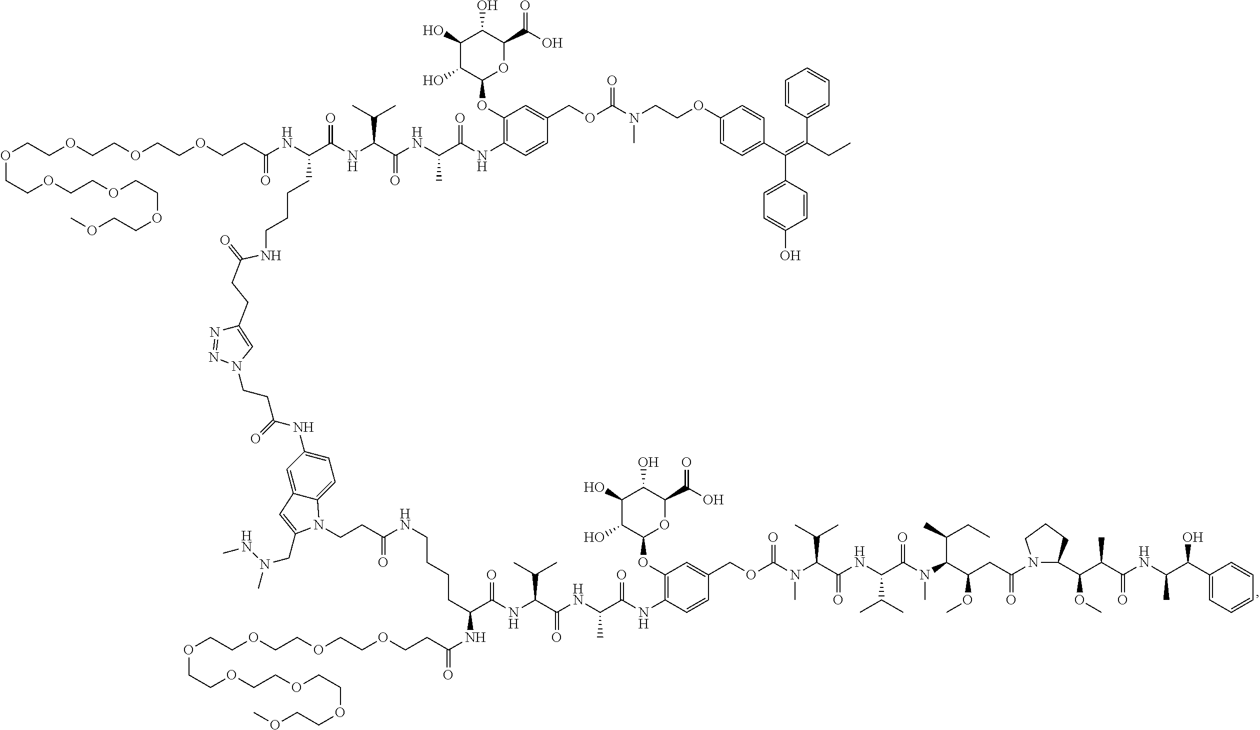

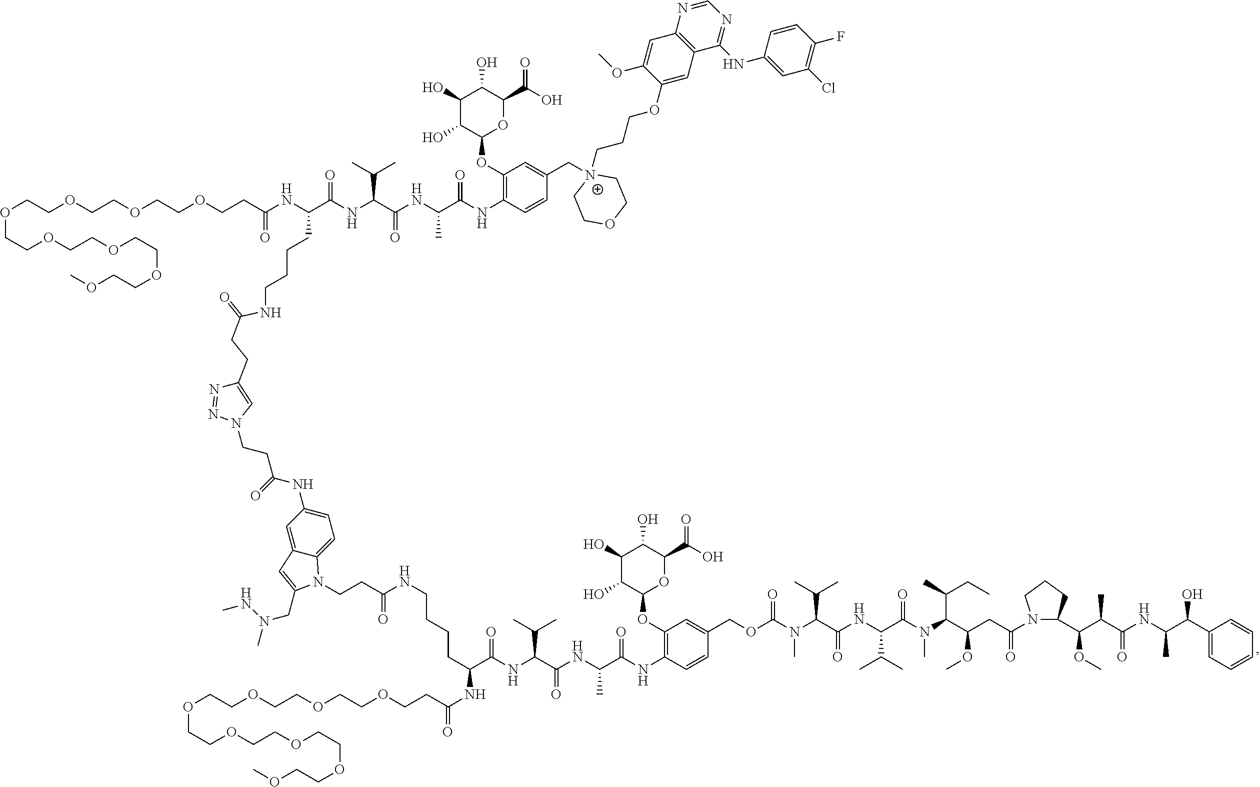

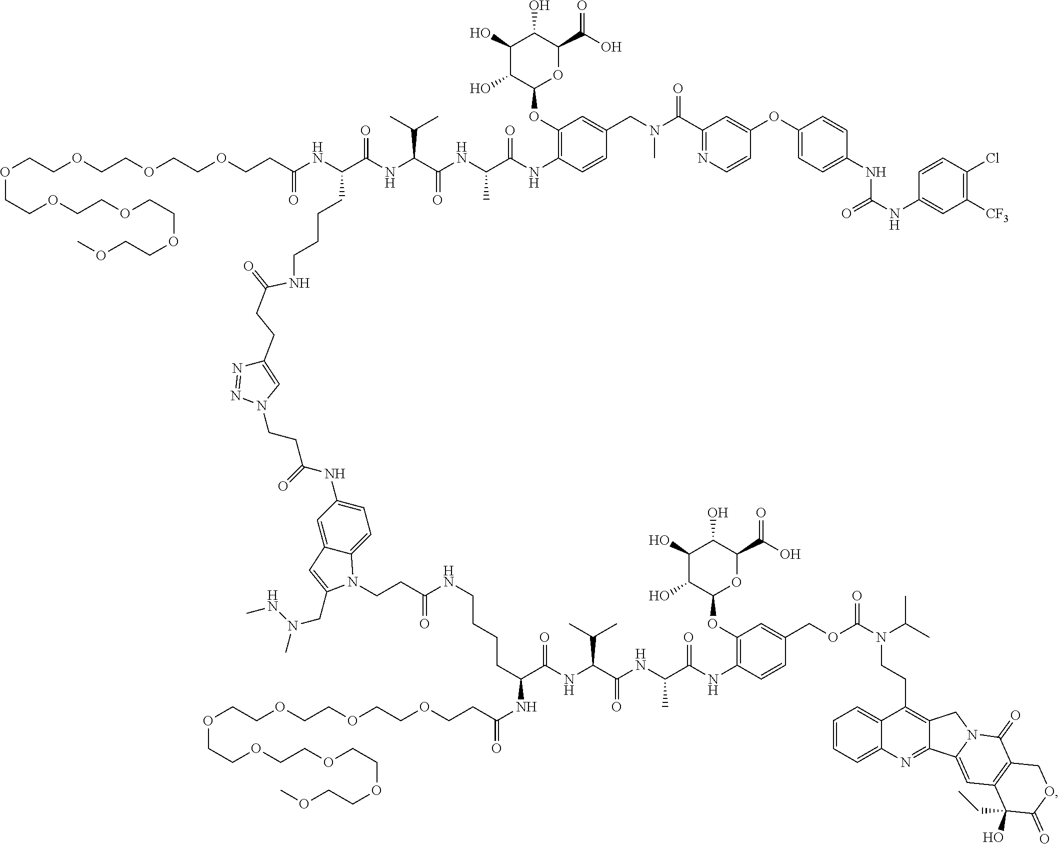 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CCCCNC(=O)CCn2c(CN(C)NC)cc3cc(NC(=O)CCn4cc(CCC(=O)NCCCC[C@H](NC(=O)CCOCCOCCOCCOCCOCCOCCOCCOC)C(=O)N[C@H](C(=O)N[C@@H](C)C(=O)Nc5ccc(CN(C)C(=O)c6cc(Oc7ccc(NC(=O)Nc8ccc(Cl)c(C(F)(F)F)c8)cc7)ccn6)cc5O[C@@H]5O[C@H](C(=O)O)[C@@H](O)[C@H](O)[C@H]5O)C(C)C)nn4)ccc32)NC(=O)CCOCCOCCOCCOCCOCCOCCOCCOC)C(C)C)c(O[C@@H]2O[C@H](C(=O)O)[C@@H](O)[C@H](O)[C@H]2O)c1)C(C)C]

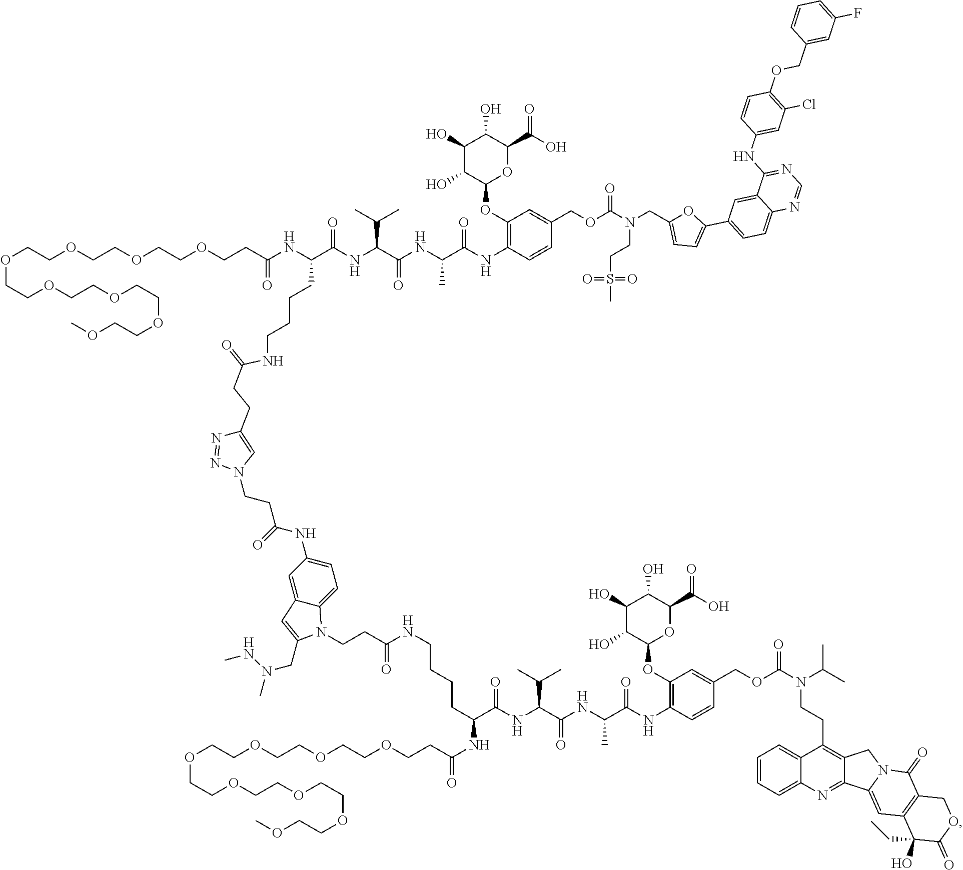 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CCCCNC(=O)CCn2c(CN(C)NC)cc3cc(NC(=O)CCn4cc(CCC(=O)NCCCC[C@H](NC(=O)CCOCCOCCOCCOCCOCCOCCOCCOC)C(=O)N[C@H](C(=O)N[C@@H](C)C(=O)Nc5ccc(COC(=O)N(CCS(C)(=O)=O)Cc6ccc(-c7ccc8ncnc(Nc9ccc(OCc%10cccc(F)c%10)c(Cl)c9)c8c7)o6)cc5O[C@@H]5O[C@H](C(=O)O)[C@@H](O)[C@H](O)[C@H]5O)C(C)C)nn4)ccc32)NC(=O)CCOCCOCCOCCOCCOCCOCCOCCOC)C(C)C)c(O[C@@H]2O[C@H](C(=O)O)[C@@H](O)[C@H](O)[C@H]2O)c1)C(C)C]

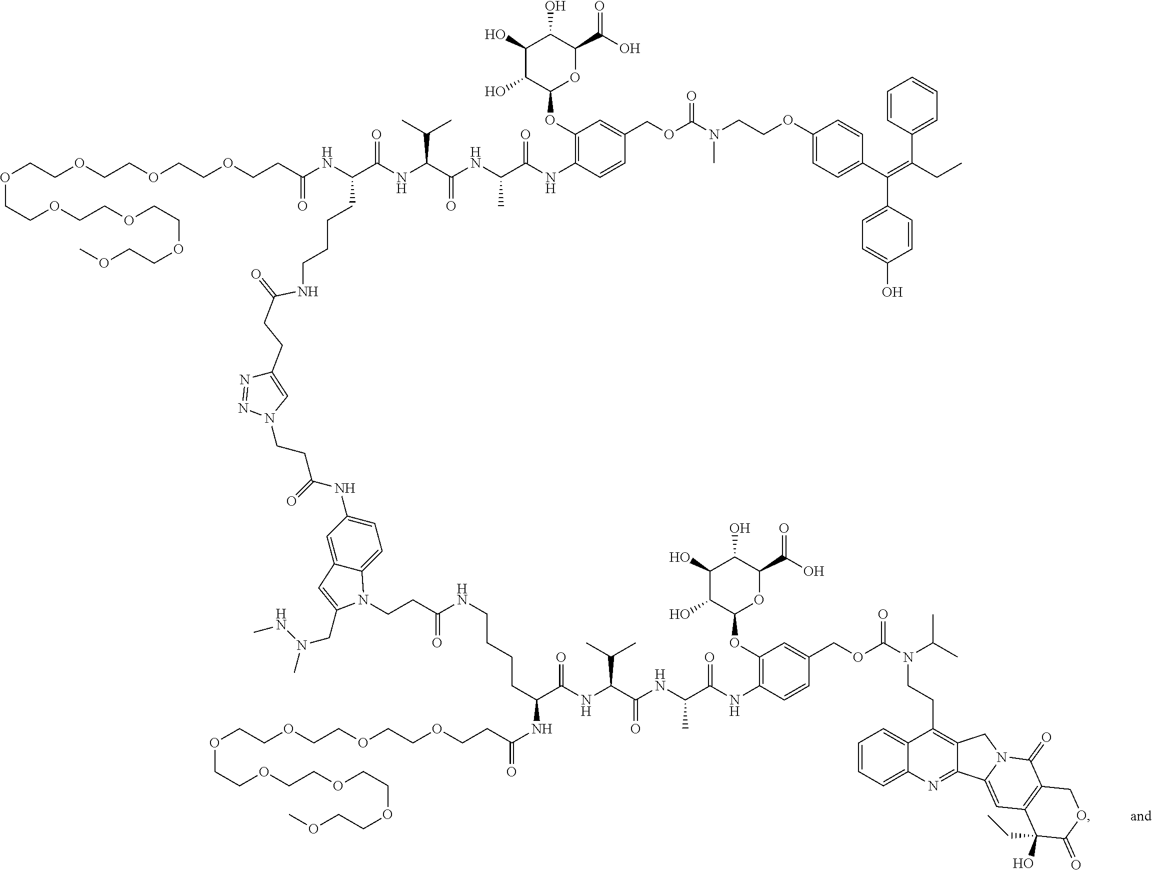 [SMILES: CC/C(=C(\c1ccc(O)cc1)c1ccc(OCCN(C)C(=O)OCc2ccc(NC(=O)[C@H](C)NC(=O)[C@@H](NC(=O)[C@H](CCCCNC(=O)CCc3cn(CCC(=O)Nc4ccc5c(c4)cc(CN(C)NC)n5CCC(=O)NCCCC[C@H](NC(=O)CCOCCOCCOCCOCCOCCOCCOCCOC)C(=O)N[C@H](C(=O)N[C@@H](C)C(=O)Nc4ccc(COC(=O)N(CCc5c6c(nc7ccccc57)-c5cc7c(c(=O)n5C6)COC(=O)[C@]7(O)CC)C(C)C)cc4O[C@@H]4O[C@H](C(=O)O)[C@@H](O)[C@H](O)[C@H]4O)C(C)C)nn3)NC(=O)CCOCCOCCOCCOCCOCCOCCOCCOC)C(C)C)c(O[C@@H]3O[C@H](C(=O)O)[C@@H](O)[C@H](O)[C@H]3O)c2)cc1)c1ccccc1 ; "and"]

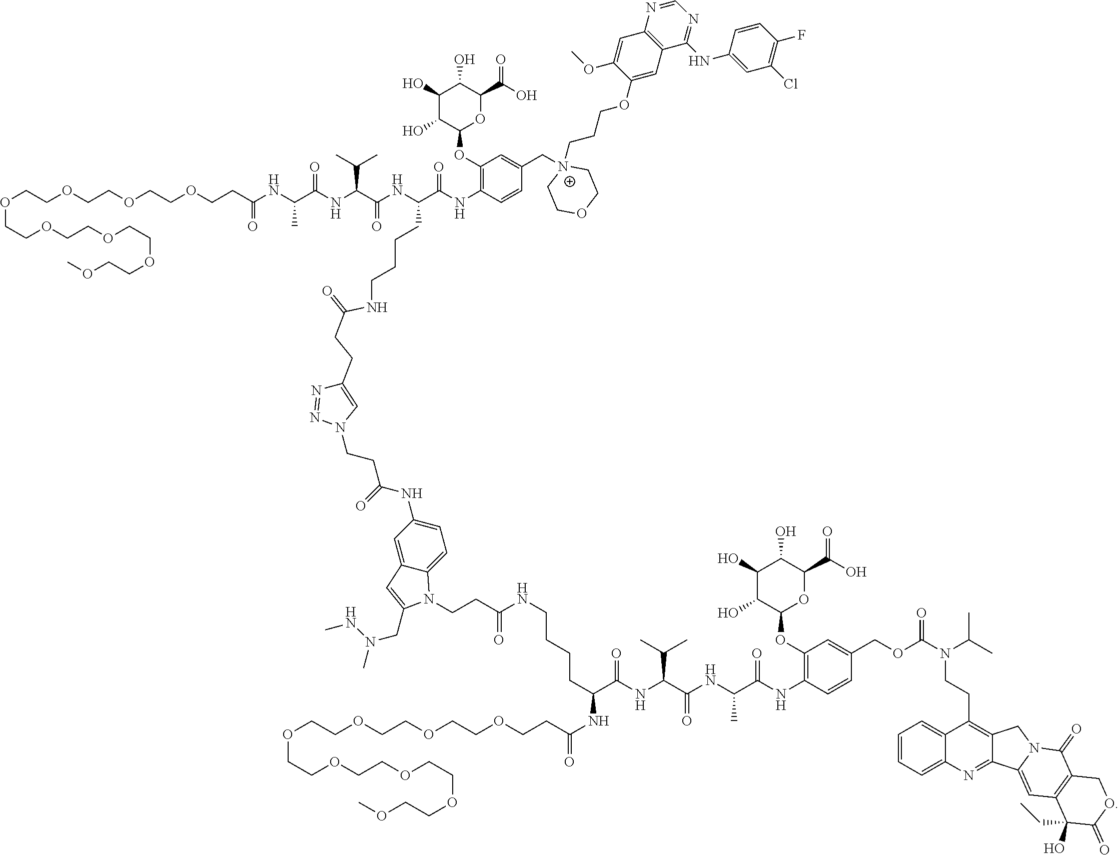 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CCCCNC(=O)CCn2c(CN(C)NC)cc3cc(NC(=O)CCn4cc(CCC(=O)NCCCC[C@H](NC(=O)[C@@H](NC(=O)[C@H](C)NC(=O)CCOCCOCCOCCOCCOCCOCCOCCOC)C(C)C)C(=O)Nc5ccc(C[N+]6(CCCOc7cc8c(Nc9ccc(F)c(Cl)c9)ncnc8cc7OC)CCOCC6)cc5O[C@@H]5O[C@H](C(=O)O)[C@@H](O)[C@H](O)[C@H]5O)nn4)ccc32)NC(=O)CCOCCOCCOCCOCCOCCOCCOCCOC)C(C)C)c(O[C@@H]2O[C@H](C(=O)O)[C@@H](O)[C@H](O)[C@H]2O)c1)C(C)C]

Any of the chemical entities, linkers and conjugation moieties set forth in the structures above may be adapted for use in the subject compounds and conjugates.

Additional disclosure related to hydrazinyl-indolyl and hydrazinyl-pyrrolo-pyridinyl compounds and methods for producing a conjugate is found in U.S. Pat. Nos. 9,310,374 and 9,493,413, the disclosures of each of which are incorporated herein by reference.

### Compounds Useful for Producing Conjugates

The present disclosure provides compounds useful for producing the conjugates described herein. In certain embodiments, the compound can be attached to two or more drugs or active agents and may also include a hydrazinyl-indolyl or hydrazinyl-pyrrolo-pyridinyl conjugation moiety useful for conjugation of the drugs or active agents to a polypeptide (e.g., an antibody). For example, the conjugation moiety in the compound may be conjugated to a polypeptide (e.g., antibody), thus indirectly binding the drugs or active agents and the polypeptide (antibody) together.

In certain embodiments, the compound is a compound of formula (II):

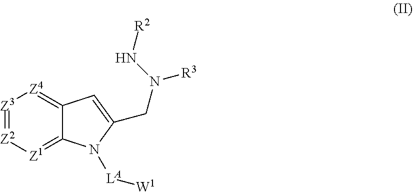

wherein:
   Z¹, Z², Z³ and Z⁴ are each independently selected from CR⁴, N and C-L^{B}-W², wherein at least one Z¹, Z², Z³ and Z⁴ is C-L^{B}-W²;
   R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
   each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
   L^{A} is a first linker;
   L^{B} is a second linker;
   W¹ is a first drug; and
   W² is a second drug.

Regarding compounds of formula (II), the substituents Z¹, Z², Z³, Z⁴, R², R³, R⁴, L^{A}, L^{B}, W¹, and W² are as described above in relation to the conjugates of formula (I). Similarly, regarding the first linker L^{A} and the second linker L^{B} of formula (II), the T¹, T², T³, T⁴, T⁵, T⁶, V¹, V², V³, V⁴, V⁵ and V⁶, and T⁷, T⁸, T⁹, T¹⁰, T¹¹, T¹², T¹³, V⁷, V⁸, V⁹, V¹⁰, V¹¹, V¹² and V¹³ substituents are as described above in relation to the conjugates of formula (I).

For example, in some instances, T¹, T², T³, T⁴, T⁵ and T⁶ and V¹, V², V³, V⁴, V⁵ and V⁶ are selected from the following:
wherein:
   T¹ is (C₁-C₁₂)alkyl and V¹ is —CO—;
   T² is AA and V² is absent;
   T³ is PABC and V³ is absent; and
   d, e and f are each 0; or
wherein:
   T¹ is (C₁-C₁₂)alkyl and V¹ is —CONH—;
   T² is (PEG)ₙ and V² is —CO—;
   T³ is AA and V³ is absent;
   T⁴ is PABC and V⁴ is absent; and
   e and f are each 0; or
wherein:
   T¹ is (C₁-C₁₂)alkyl and V¹ is —CO—;
   T² is an amino acid analog and V² is —NH—;
   T³ is (PEG)ₙ and V³ is —CO—;
   T⁴ is AA and V⁴ is absent;
   T⁵ is PABC and V⁵ is absent; and
   f is 0; or
wherein:
   T¹ is (C₁-C₁₂)alkyl and V¹ is —CONH—;
   T² is (PEG)ₙ and V² is —CO—;
   T³ is AA and V³ is absent;
   T⁴ is PABC and V⁴ is absent; and
   e and f are each 0; or
wherein:
   T¹ is (C₁-C₁₂)alkyl and V¹ is —CONH—;
   T² is substituted (C₁-C₁₂)alkyl and V² is —CO—;
   T³ is AA and V³ is absent;
   T⁴ is PABC and V⁴ is absent; and
   e and f are each 0; or
wherein:
   T¹ is (C₁-C₁₂)alkyl and V¹ is —CONH—;
   T² is (PEG)ₙ and V² is —CO—;
   T³ is AA and V³ is absent;
   T⁴ is PABA and V⁴ is —CO—;
   T⁵ is (C₁-C₁₂)alkyl and V⁵ is absent; and
   f is 0; or
wherein:
   T¹ is (C₁-C₁₂)alkyl and V¹ is —CO—;
   T² is 4AP and V² is —CO—;
   T³ is (C₁-C₁₂)alkyl and V³ is —CO—;
   T⁴ is AA and V⁴ is absent;
   T⁵ is PABC and V⁵ is absent; and
   f is 0; or
wherein:
   T¹ is (C₁-C₁₂)alkyl and V¹ is —CO—;
   T² is 4AP and V² is —CO—;
   T³ is (C₁-C₁₂)alkyl and V³ is —O—;
   T⁴ is (C₁-C₁₂)alkyl and V⁴ is —CO—;
   T⁵ is AA and V⁵ is absent; and
   T⁶ is PABC and V⁶ is absent; or
wherein:
   T¹ is (C₁-C₁₂)alkyl and V¹ is —CO—;
   T² is an amino acid analog and V² is absent;
   T³ is AA and V³ is absent;
   T⁴ is PABC and V⁴ is absent; and
   e and f are each 0; or
wherein:
   T¹ is (C₁-C₁₂)alkyl and V¹ is —CONH—;
   T² is (PEG)ₙ and V² is —CONH—;
   T³ is substituted (C₁-C₁₂)alkyl and V³ is —CO—;
   T⁴ is AA and V⁴ is absent;
   T⁵ is PABC and V⁵ is absent; and
   f is 0; or
wherein:
   T¹ is (C₁-C₁₂)alkyl and V¹ is —CO—;
   T² is an AA and V² is —NH—;
   T³ is (PEG)ₙ and V³ is —CO—;
   T⁴ is AA and V⁴ is absent;
   T⁵ is PABC and V⁵ is absent; and
   f is 0; or
wherein:
   T¹ is (C₁-C₁₂)alkyl and V¹ is —CONH—;
   T² is (PEG)ₙ and V² is —CO—;
   T³ is AA and V³ is absent;
   T⁴ is PAP and V⁴ is —C(O)O—; and
   e and f are each 0; or
wherein:
   T¹ is (C₁-C₁₂)alkyl and V¹ is —CONH—;
   T² is substituted (C₁-C₁₂)alkyl and V² is —CO—;
   T³ is AA and V³ is absent;
   T⁴ is PABC and V⁴ is absent; and
   e and f are each 0; or
wherein:
   T¹ is (C₁-C₁₂)alkyl and V¹ is —CONH—;
   T² is substituted (C₁-C₁₂)alkyl and V² is —CO—;
   T³ is PABC and V³ is absent; and
   d, e and f are each 0.

For example, in some instances, T⁷, T⁸, T⁹, T¹⁰, T¹¹, T¹² and T¹³ and V⁷, V⁸, V⁹, V¹⁰, V¹¹, V¹² and V¹³ are selected from the following:
wherein:
   T⁷ is absent and V⁷ is —NHCO—;
   T⁸ is (C₁-C₁₂)alkyl and V⁸ is —CO—;
   T⁹ is AA and V⁹ is absent;
   T¹⁰ is PABC and V¹⁰ is absent; and
   k, l and m are each 0; or
wherein:
   T⁷ is absent and V⁷ is —NHCO—;
   T⁸ is (C₁-C₁₂)alkyl and V⁸ is —CONH—;
   T⁹ is (PEG)ₙ and V⁹ is —CO—;
   T¹⁰ is AA and V¹⁰ is absent; and
   T¹¹ is PABC and V¹¹ is absent; and
   l and m are each 0; or
wherein:
   T⁷ is absent and V⁷ is —NHCO—;
   T⁸ is (C₁-C₁₂)alkyl and V⁸ is —CO—;
   T⁹ is an amino acid analog and V⁹ is —NH—;
   T¹⁰ is (PEG)ₙ and V¹⁰ is —CO—;
   T¹¹ is AA and V¹¹ is absent;
   T¹² is PABC and V¹² is absent; and
   m is 0; or
wherein:
   T⁷ is absent and V⁷ is —NHCO—;
   T⁸ is (C₁-C₁₂)alkyl and V⁸ is —CONH—;
   T⁹ is (PEG)ₙ and V⁹ is —CO—;
   T¹⁰ is AA and V¹⁰ is absent;
   T¹¹ is PABC and V¹¹ is absent; and
   l and m are each 0; or
wherein:
   T⁷ is absent and V⁷ is —NHCO—;
   T⁸ is (C₁-C₁₂)alkyl and V⁸ is —CONH—;
   T⁹ is substituted (C₁-C₁₂)alkyl and V⁹ is —CO—;
   T¹⁰ is AA and V¹⁰ is absent;
   T¹¹ is PABC and V¹¹ is absent; and
   l and m are each 0; or
wherein:
   T⁷ is absent and V⁷ is —NHCO—;
   T⁸ is (C₁-C₁₂)alkyl and V⁸ is —CONH—;
   T⁹ is (PEG)ₙ and V⁹ is —CO—;
   T¹⁰ is AA and V¹⁰ is absent;
   T¹¹ is PABA and V¹¹ is —CO—;
   T¹² is (C₁-C₁₂)alkyl and V¹² is absent; and
   m is 0; or
wherein:
   T⁷ is absent and V⁷ is —NHCO—;
   T⁸ is (C₁-C₁₂)alkyl and V⁸ is —CO—;
   T⁹ is 4AP and V⁹ is —CO—;
   T¹⁰ is (C₁-C₁₂)alkyl and V¹⁰ is —CO—;
   T¹¹ is AA and V¹¹ is absent;
   T¹² is PABC and V¹² is absent; and
   m is 0; or
wherein:
   T⁷ is absent and V⁷ is —NHCO—;
   T⁸ is (C₁-C₁₂)alkyl and V⁸ is —CO—;
   T⁹ is 4AP and V⁹ is —CO—;
   T¹⁰ is (C₁-C₁₂)alkyl and V¹⁰ is —O—;
   T¹¹ is (C₁-C₁₂)alkyl and V¹¹ is —CO—;
   T¹² is AA and V¹² is absent; and
   T¹³ PABC and V¹³ is absent; or
wherein:
   T⁷ is absent and V⁷ is —NHCO—;
   T⁸ is (C₁-C₁₂)alkyl and V⁸ is —CO—;
   T⁹ is an amino acid analog and V⁹ is absent;
   T¹⁰ is AA and V¹⁰ is absent;
   T¹¹ is PABC and V¹¹ is absent; and
   l and m are each 0; or
wherein:
   T⁷ is absent and V⁷ is —NHCO—;
   T⁸ is (C₁-C₁₂)alkyl and V⁸ is —CONH—;
   T⁹ is (PEG)ₙ and V⁹ is —CONH—;
   T¹⁰ is substituted (C₁-C₁₂)alkyl and V¹⁰ is —CO—;
   T¹¹ is AA and V¹¹ is absent;
   T¹² is PABC and V¹² is absent; and
   m is 0; or
wherein:
   T⁷ is absent and V⁷ is —NHCO—;
   T⁸ is (C₁-C₁₂)alkyl and V⁸ is —CO—;
   T⁹ is AA and V⁹ is —NH—;
   T¹⁰ is (PEG)ₙ and V¹⁰ is —CO—;
   T¹¹ is AA and V¹¹ is absent;
   T¹² is PABC and V¹² is absent; and
   m is 0; or
wherein:
   T⁷ is absent and V⁷ is —NHCO—;
   T⁸ is (C₁-C₁₂)alkyl and V⁸ is —CONH—;
   T⁹ is (PEG)ₙ and V⁹ is —CO—;
   T¹⁰ is AA and V¹⁰ is absent;
   T¹¹ is PAP and V¹¹ is —C(O)O—; and
   l and m are each 0; or
wherein:
   T⁷ is absent and V⁷ is —NHCO—;
   T⁸ is (C₁-C₁₂)alkyl and V⁸ is —CO—;
   T⁹ is AA and V⁹ is absent;
   T¹⁰ is PABC and V¹⁰ is absent;
   T¹¹ is PAP and V¹¹ is —C(O)O—; and
   l and m are each 0; or
wherein:
   T⁷ is absent and V⁷ is —NHCO—;
   T⁸ is (C₁-C₁₂)alkyl and V⁸ is —CONH—;
   T⁹ is substituted (C₁-C₁₂)alkyl and V⁹ is —CO—;
   T¹⁰ is PABC and V¹⁰ is absent; and
   k, l and m are each 0; or
wherein:
   T⁷ is absent and V⁷ is —NHCO—;
   T⁸ is (C₁-C₁₂)alkyl and V⁸ is absent;
   T⁹ is heteroaryl and V⁹ is absent;
   T¹⁰ is (C₁-C₁₂)alkyl and V¹⁰ is —CONH—;
   T¹¹ is (PEG)ₙ and V¹¹ is —CO—; and
   l and m are each 0; or
wherein:
   T⁷ is absent and V⁷ is —NHCO—;
   T⁸ is (C₁-C₁₂)alkyl and V⁸ is absent;
   T⁹ is heteroaryl and V⁹ is absent;
   T¹⁰ is (C₁-C₁₂)alkyl and V¹⁰ is —CONH—;
   T¹¹ is substituted (C₁-C₁₂)alkyl and V¹¹ is —CO—;
   T¹² is AA and V¹² is absent; and
   T¹³ PAB and V¹³ is absent; or
wherein:
   T⁷ is absent and V⁷ is —NHCO—;
   T⁸ is (C₁-C₁₂)alkyl and V⁸ is absent;
   T⁹ is heteroaryl and V⁹ is absent;
   T¹⁰ is (C₁-C₁₂)alkyl and V¹⁰ is —CONH—;
   T¹¹ is substituted (C₁-C₁₂)alkyl and V¹¹ is —CO—;
   T¹² is AA and V¹² is absent; and
   T¹³ PABC and V¹³ is absent.

Compounds of formula (II) can be used in conjugation reactions described herein, where two or more drugs or active agents attached to a hydrazinyl-indolyl or a hydrazinyl-pyrrolo-pyridinyl conjugation moiety is conjugated to a polypeptide (e.g., antibody) to form an antibody-drug conjugate.

Examples of compounds according to the present disclosure include, but are not limited to, the following structures:

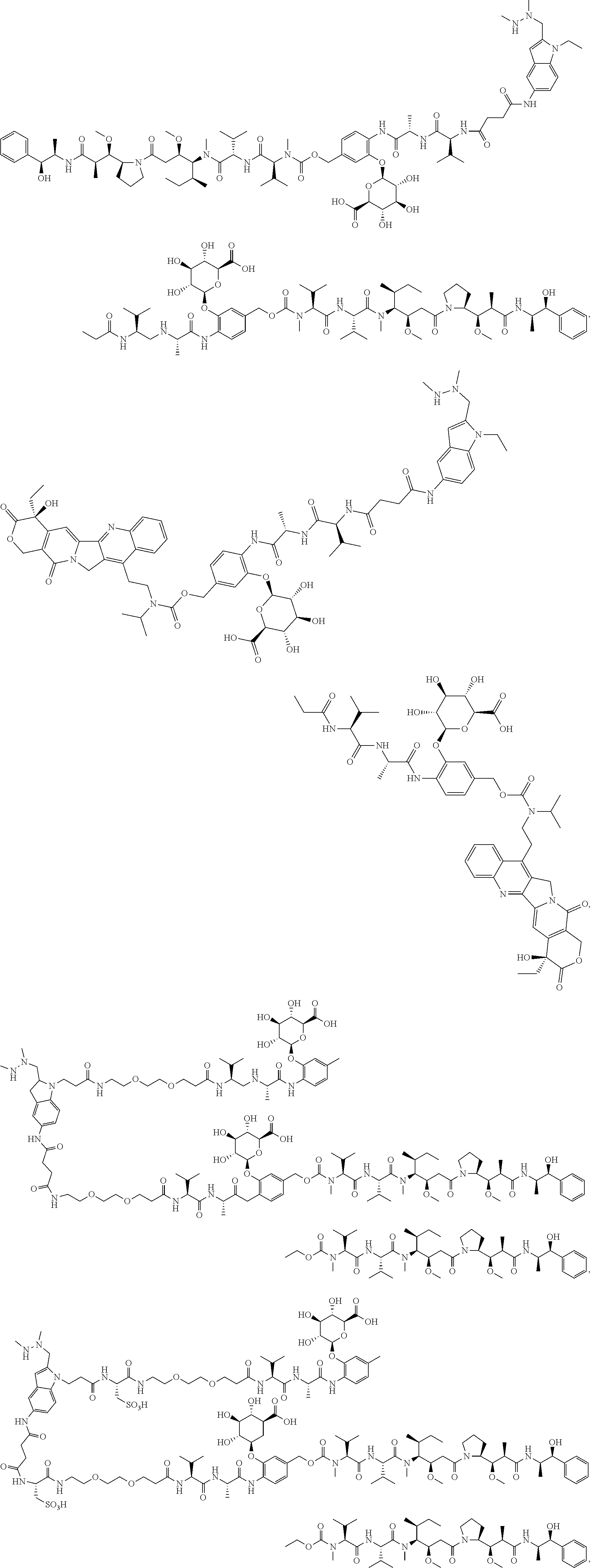

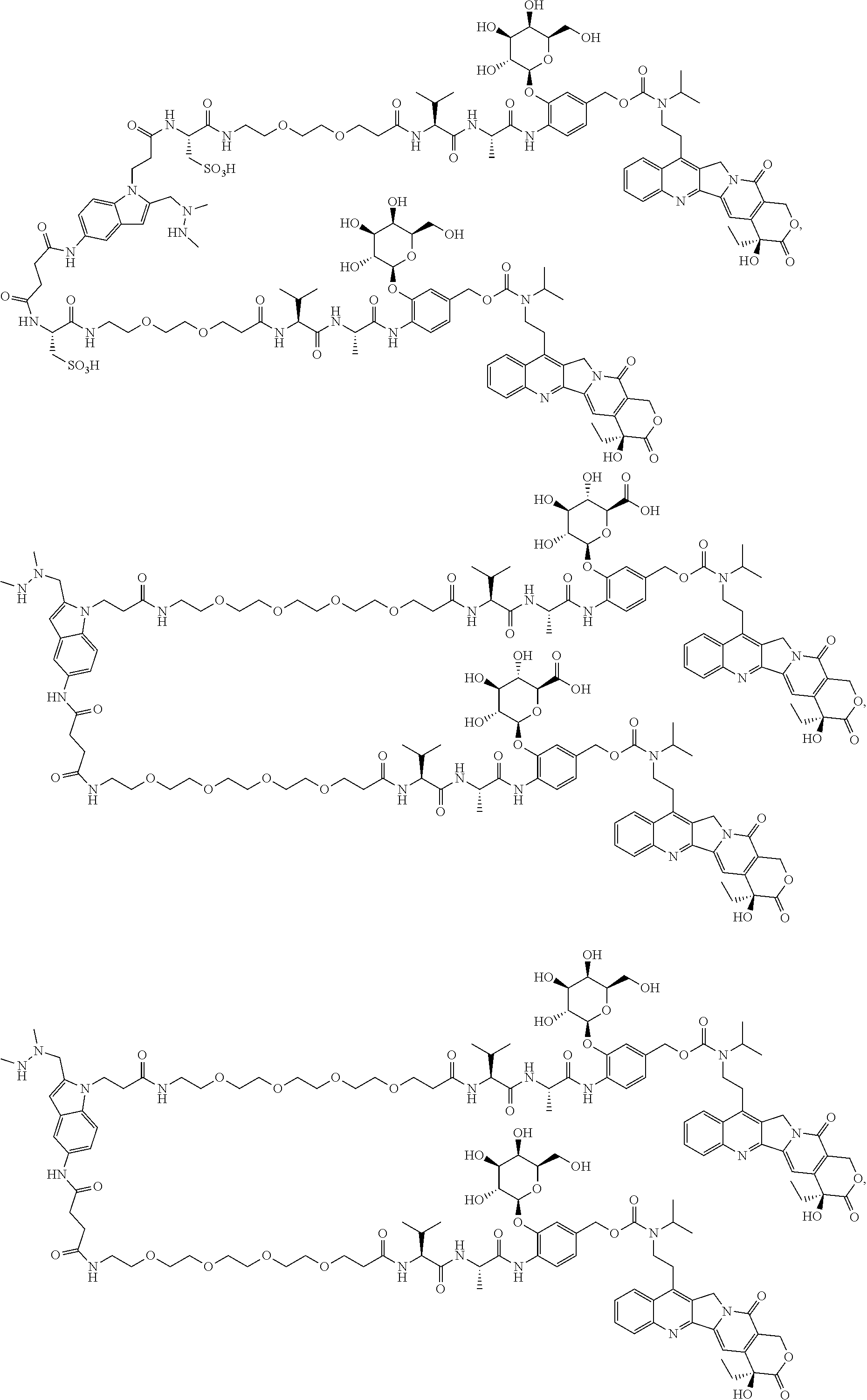

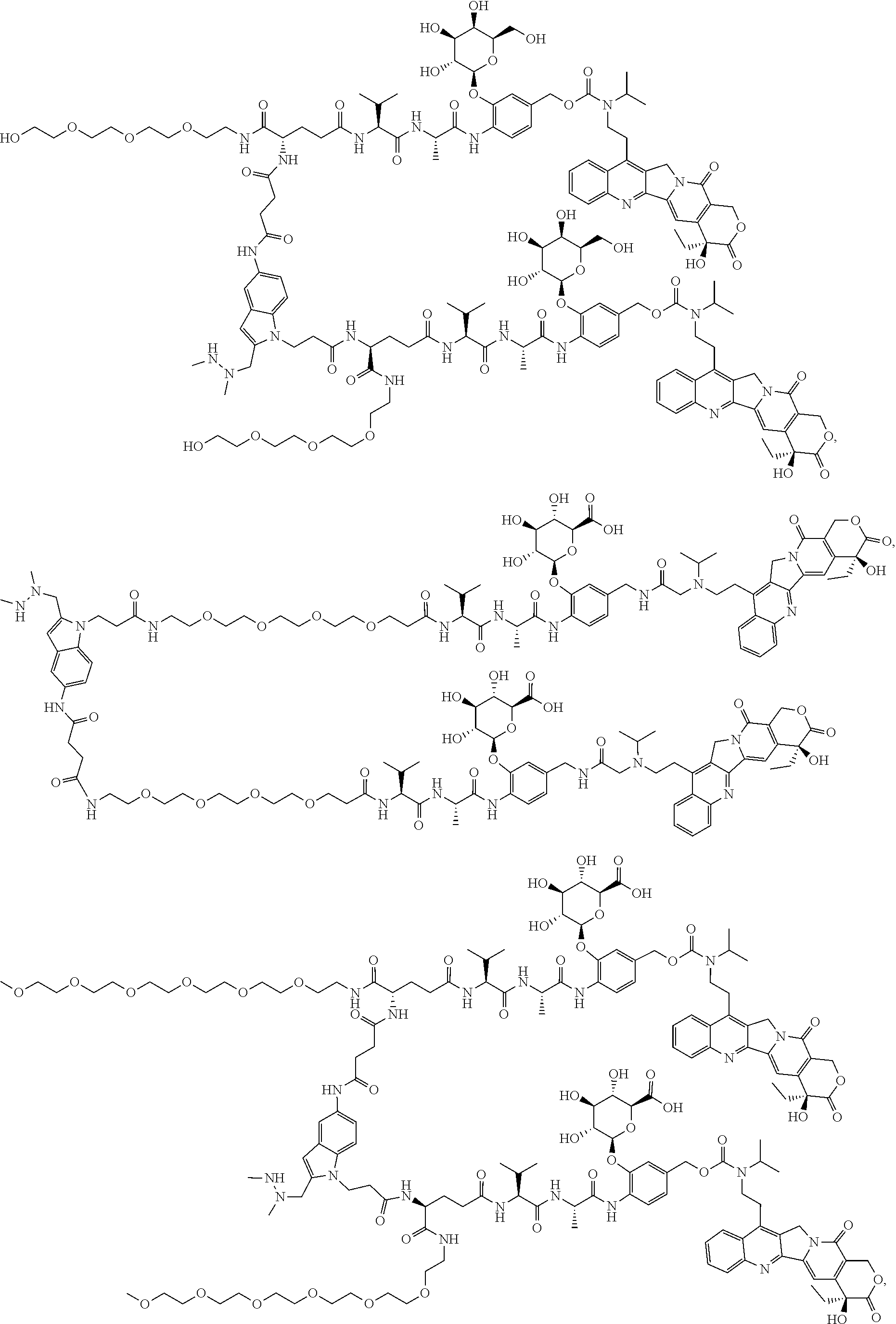

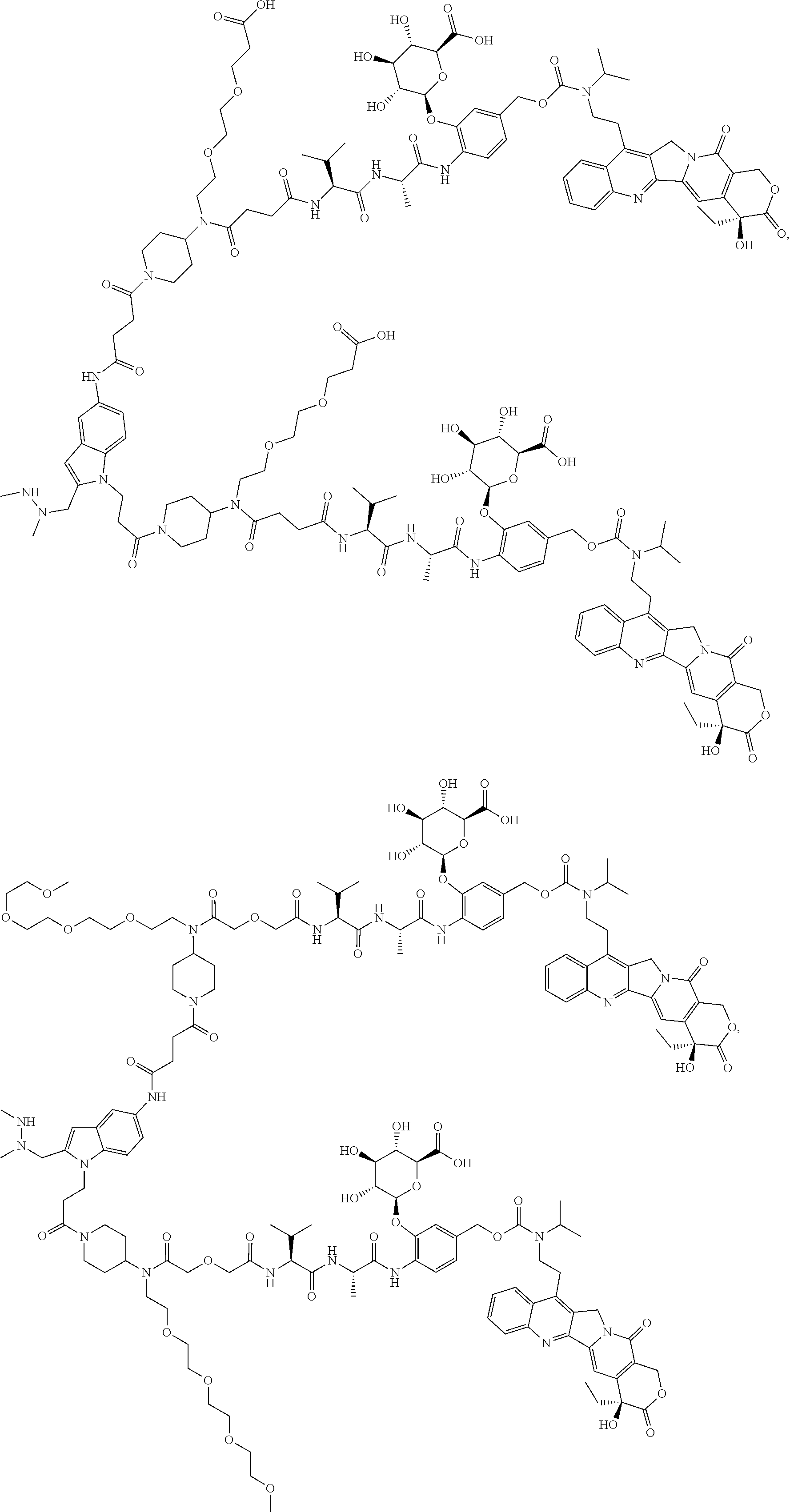

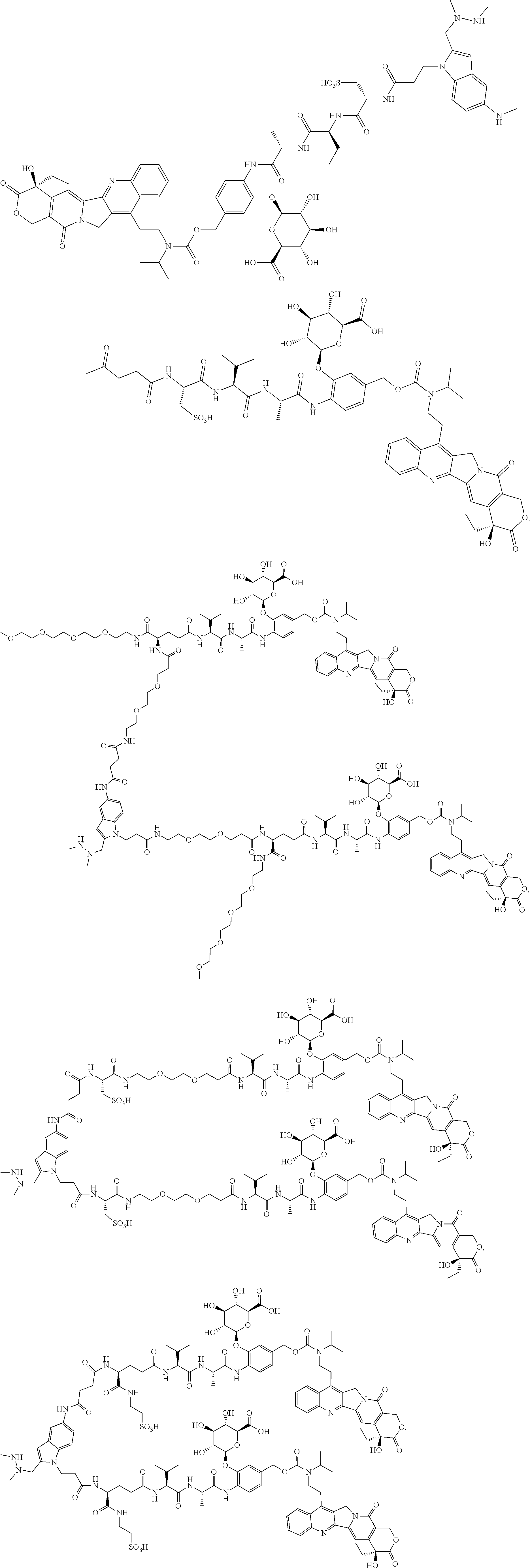

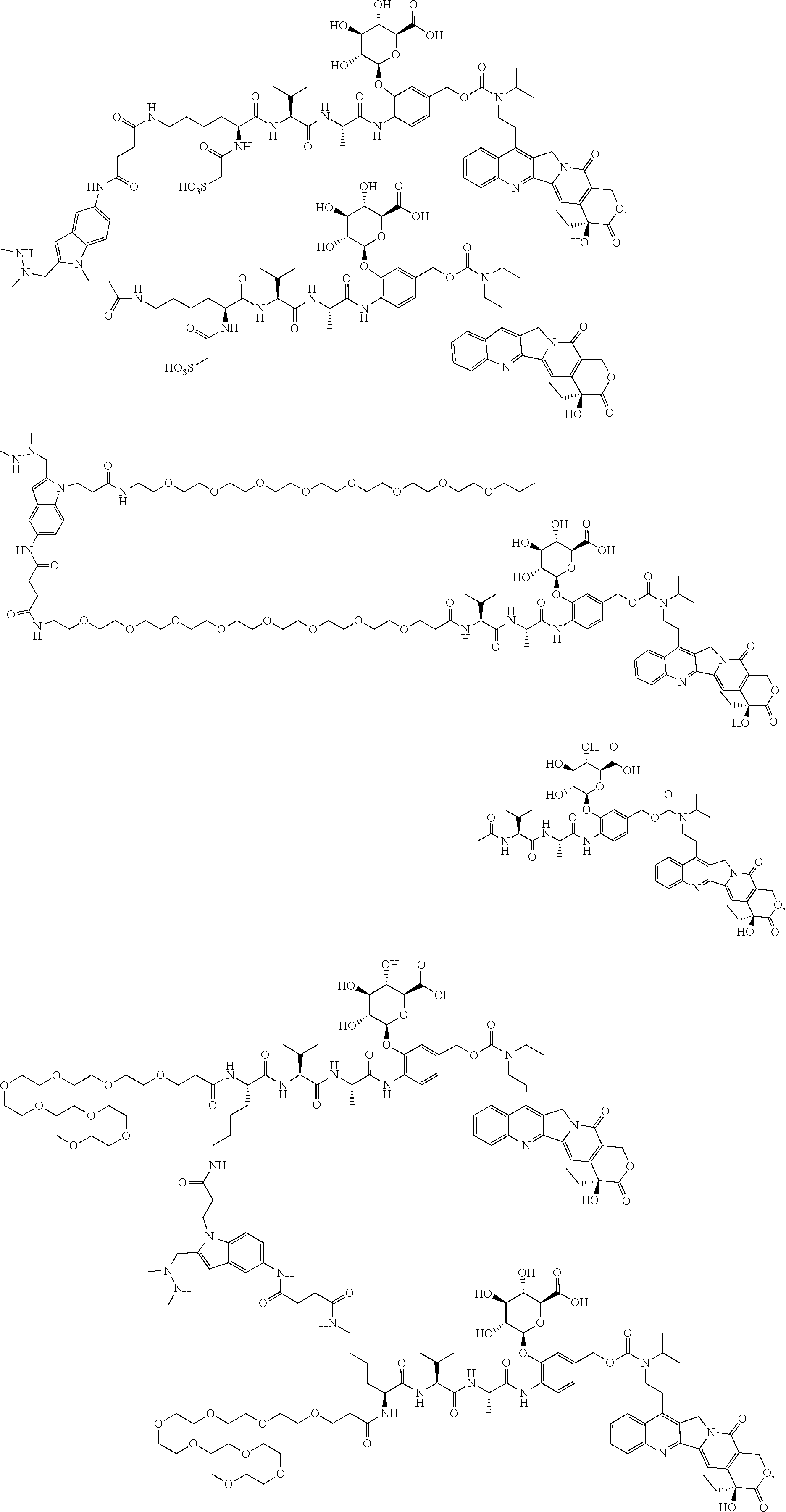

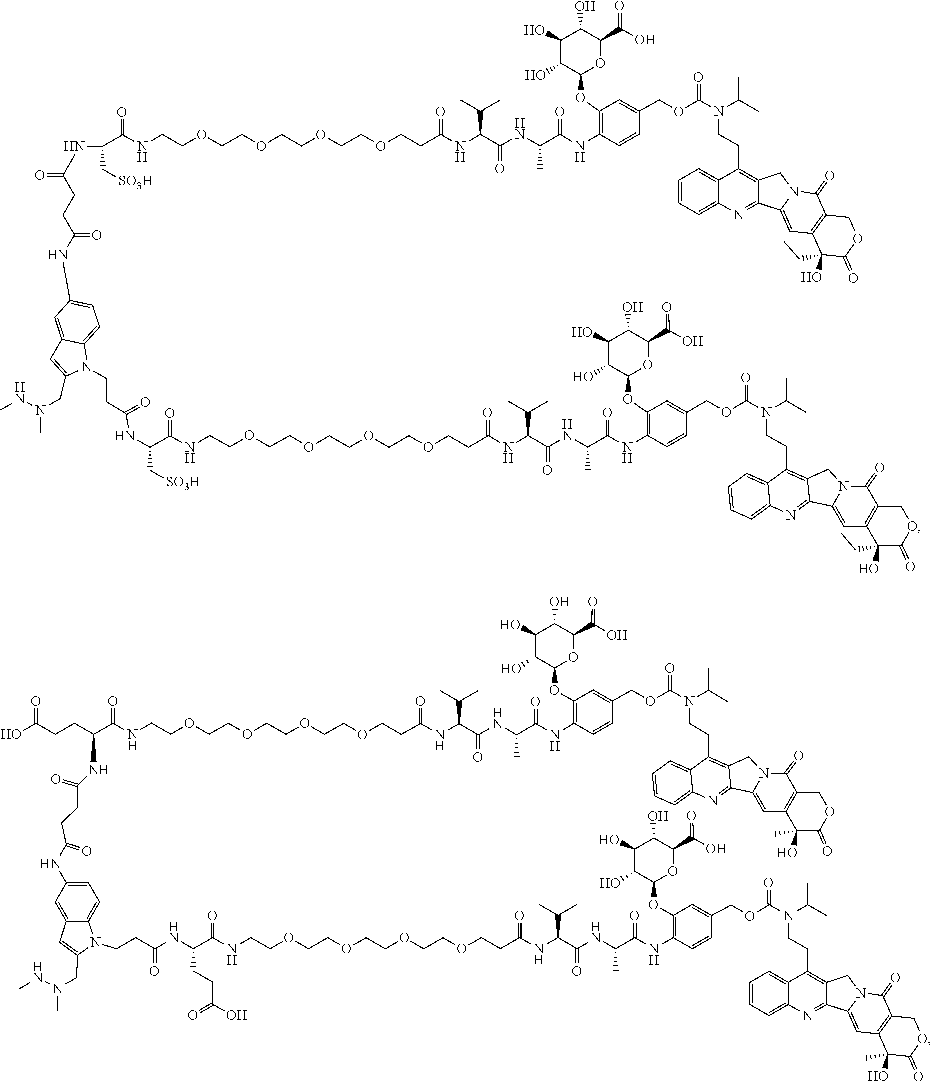

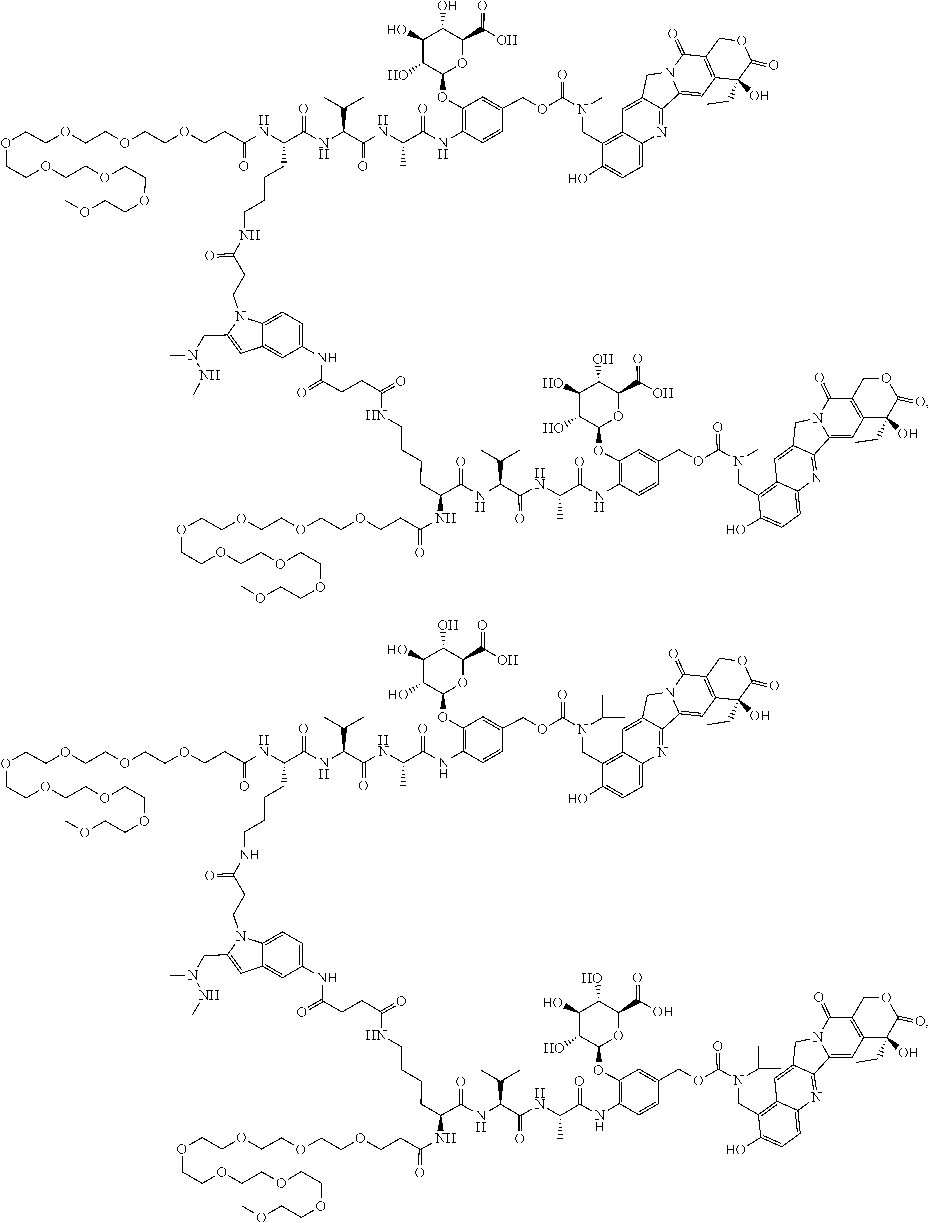

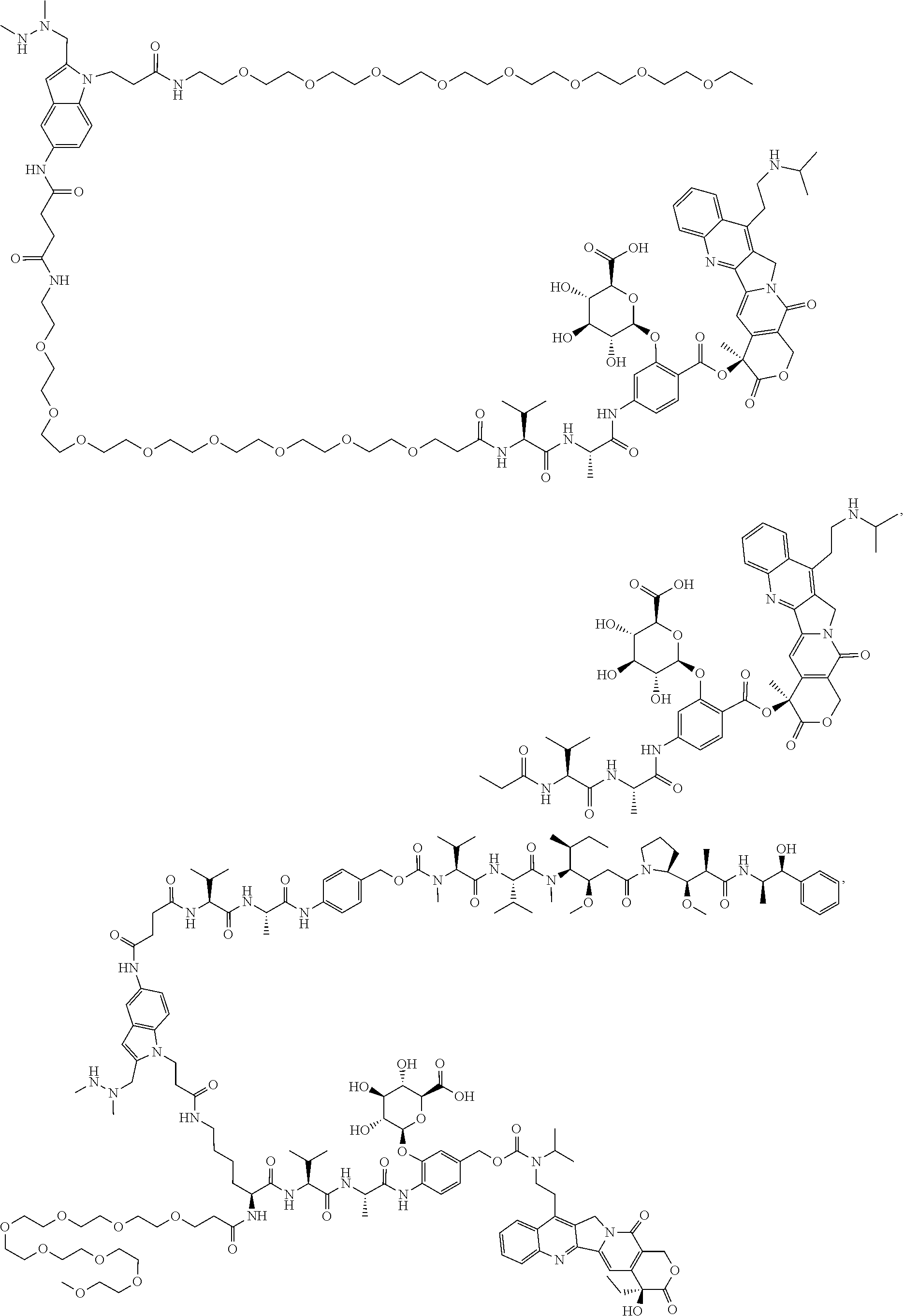

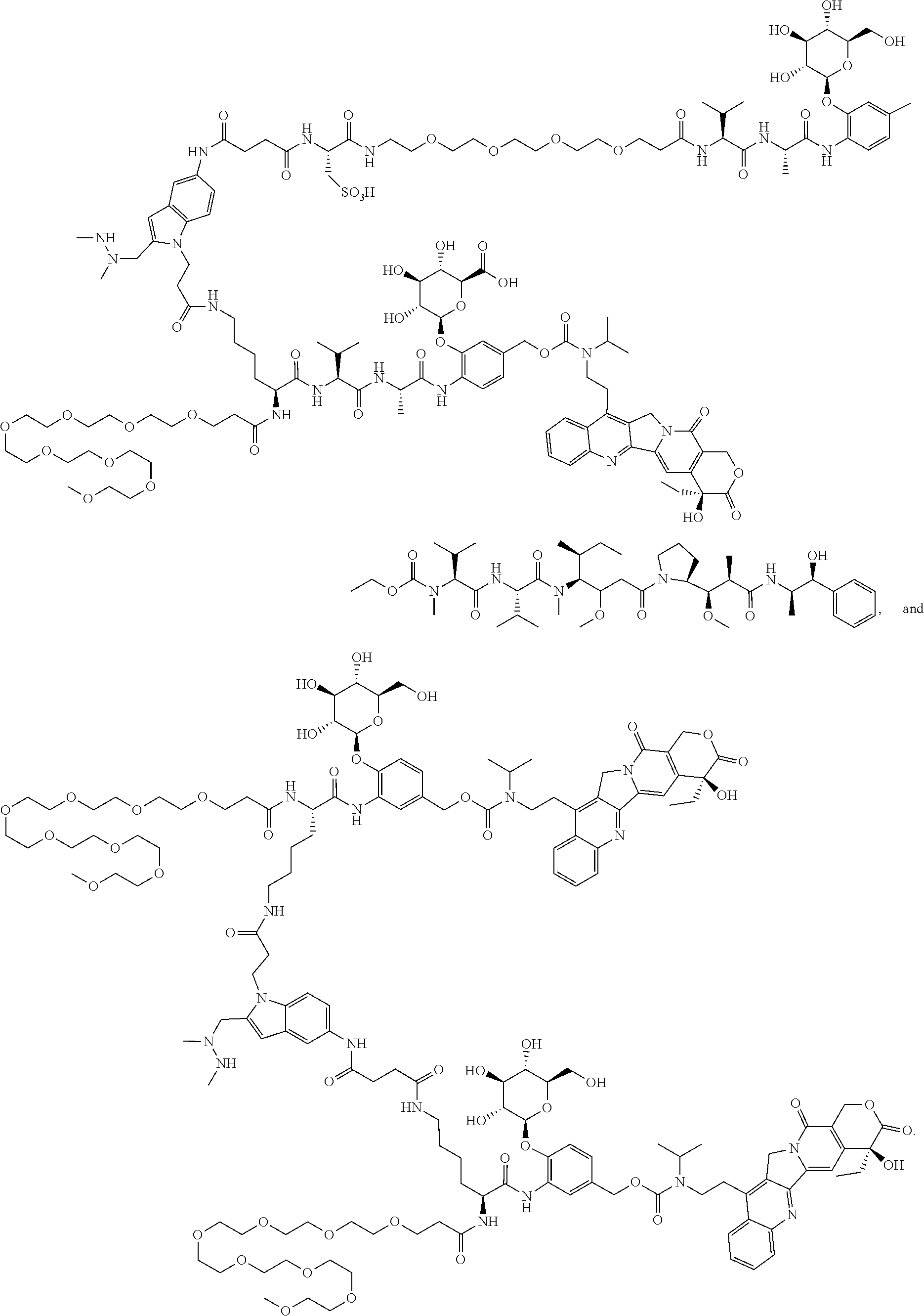

Examples of compounds according to the present disclosure include, but are not limited to, the following structures:

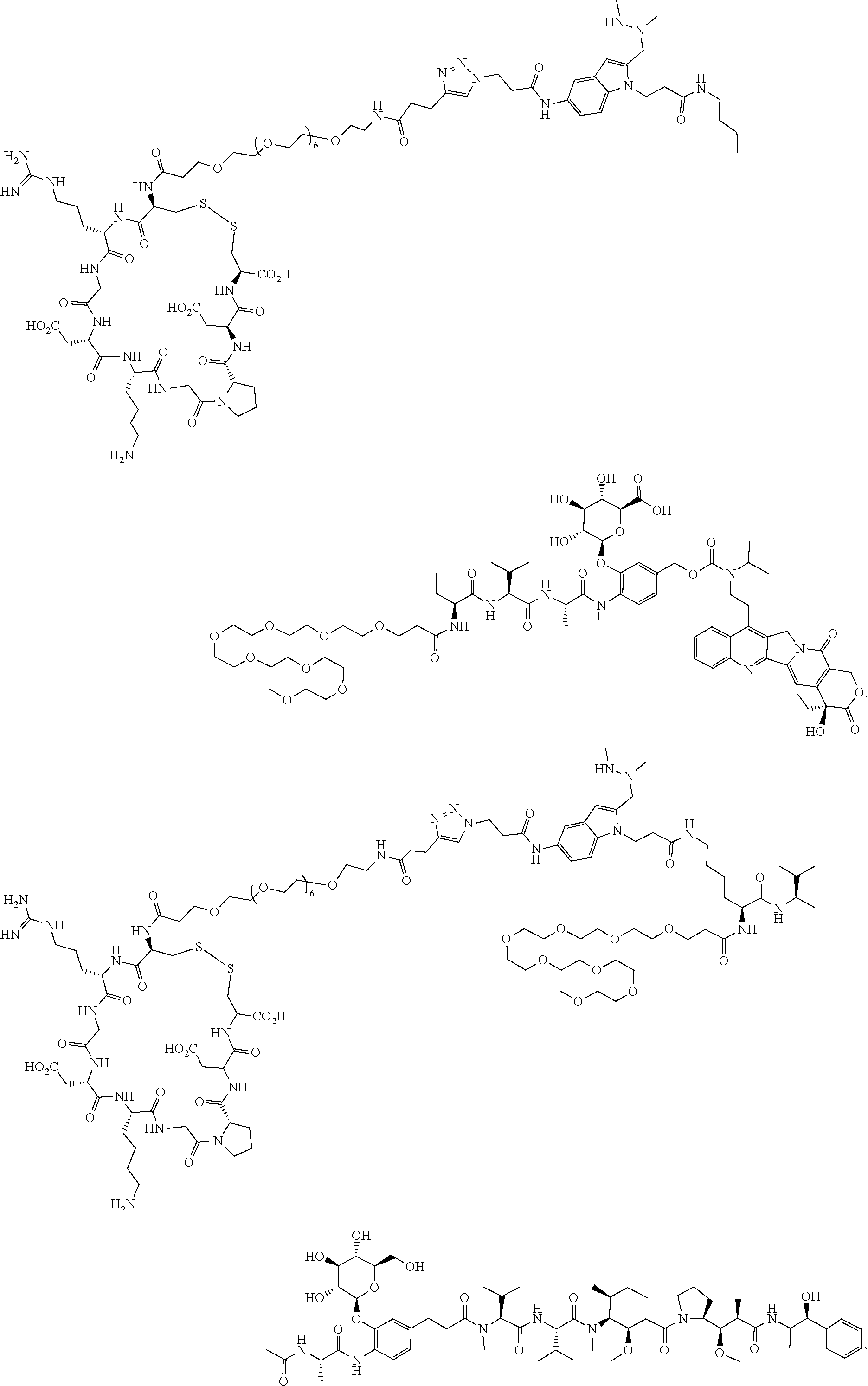

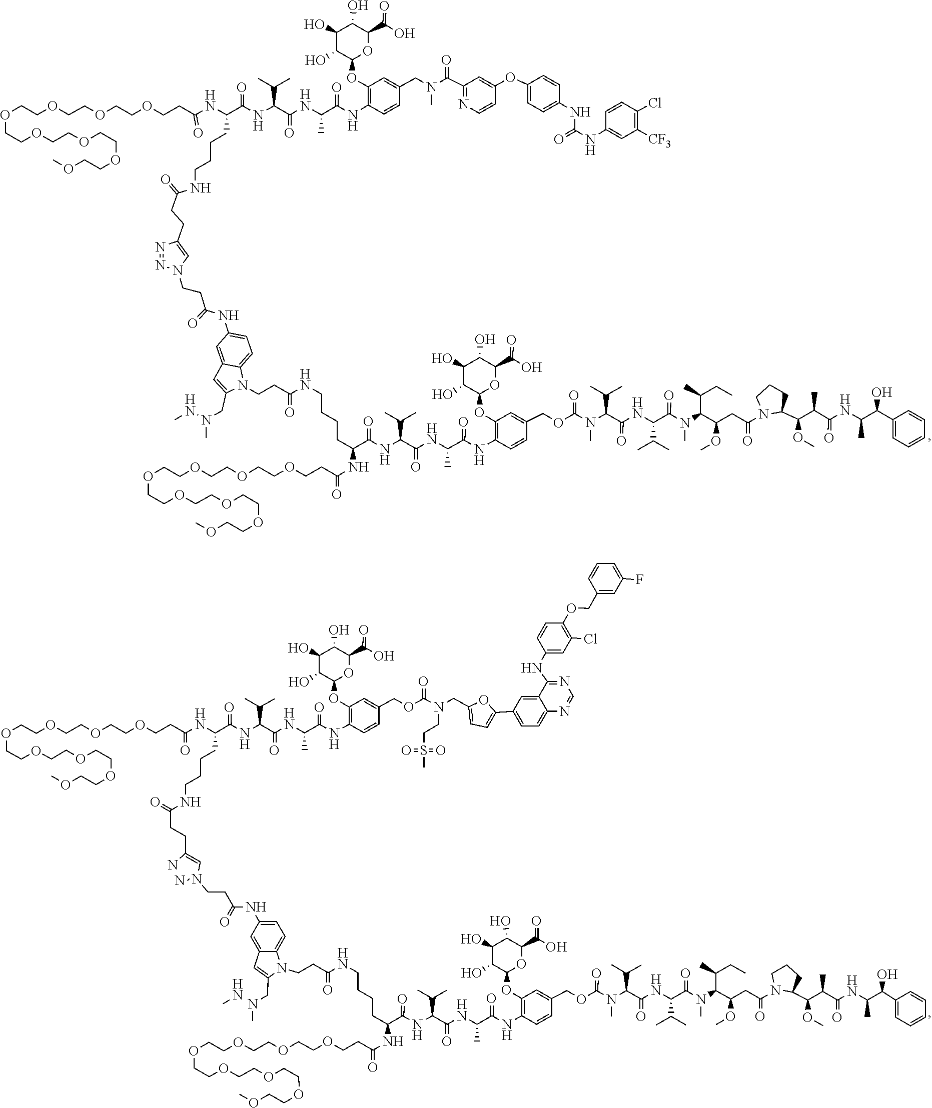

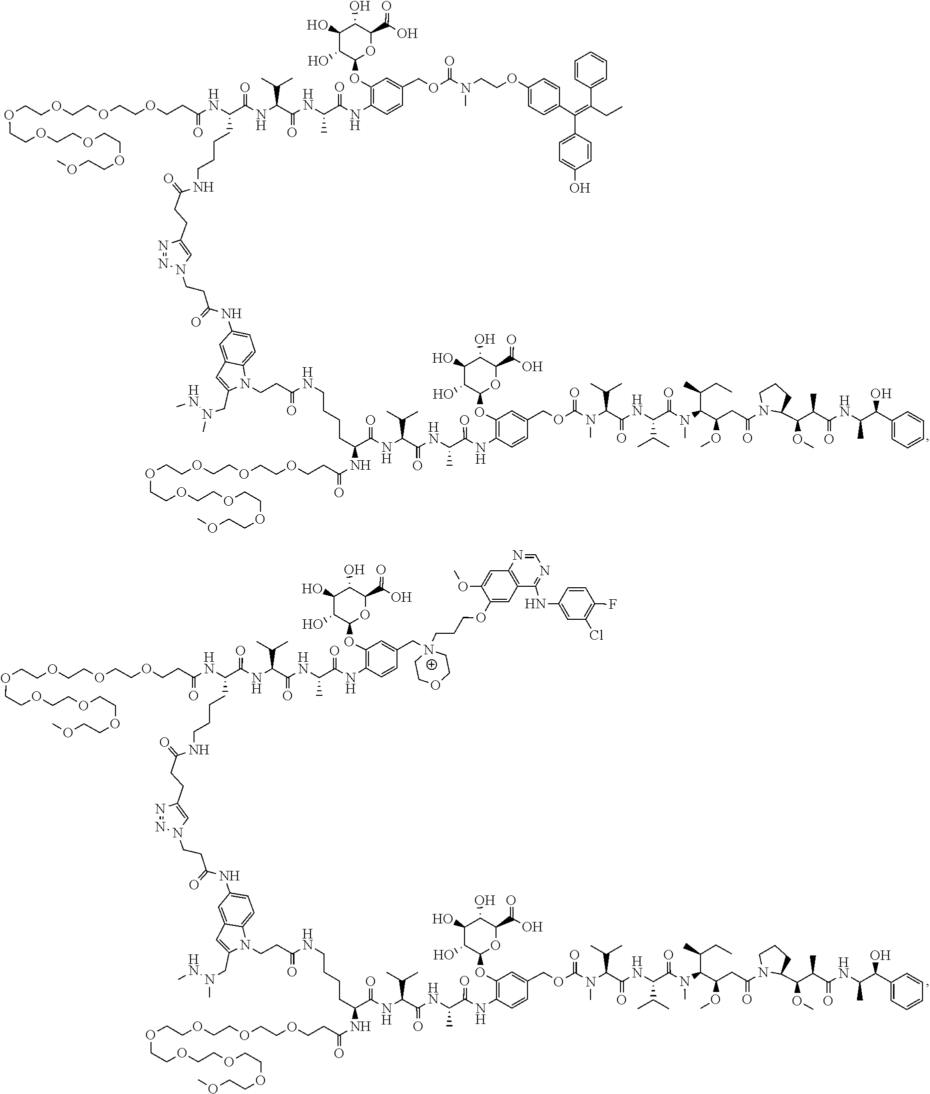

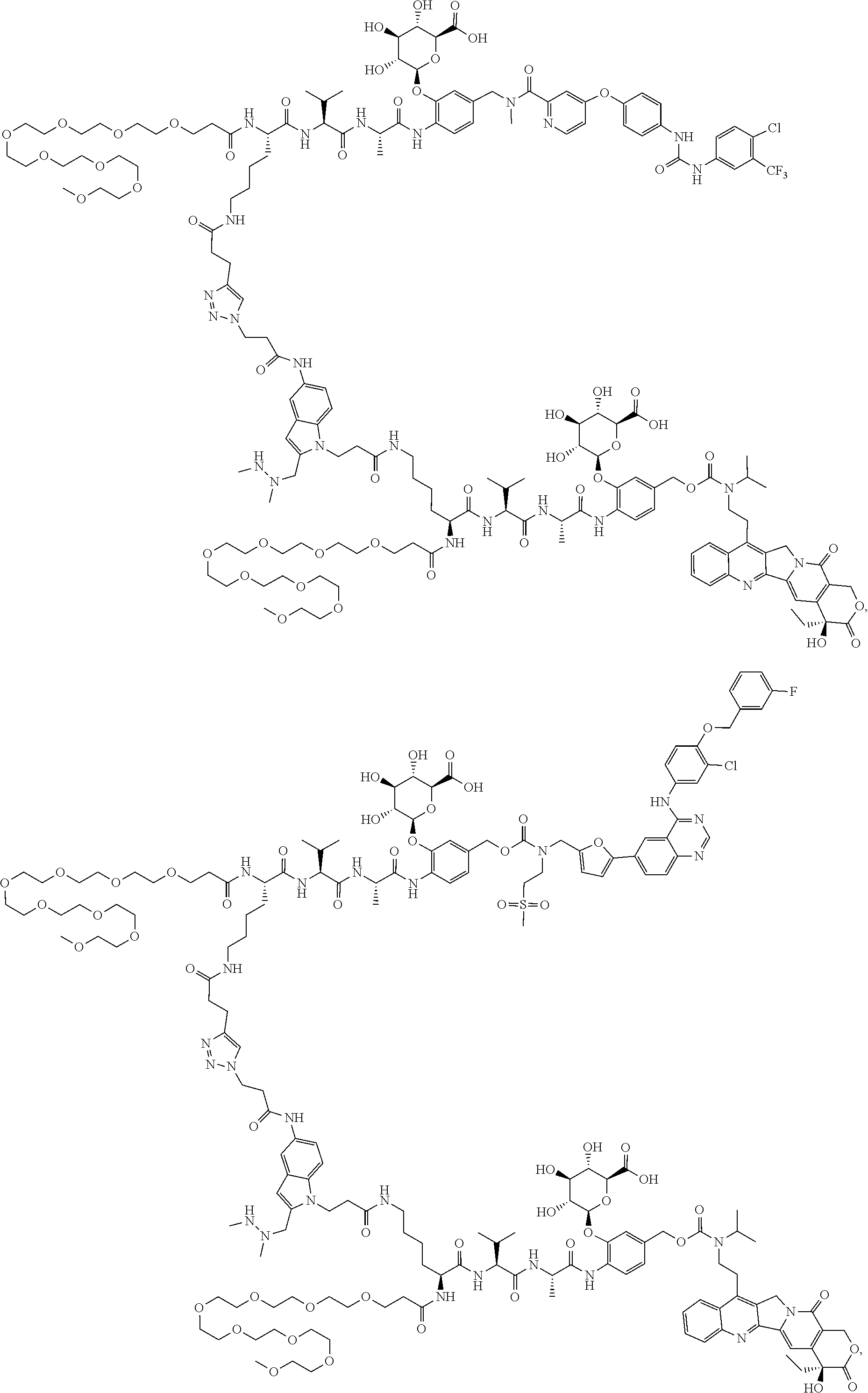

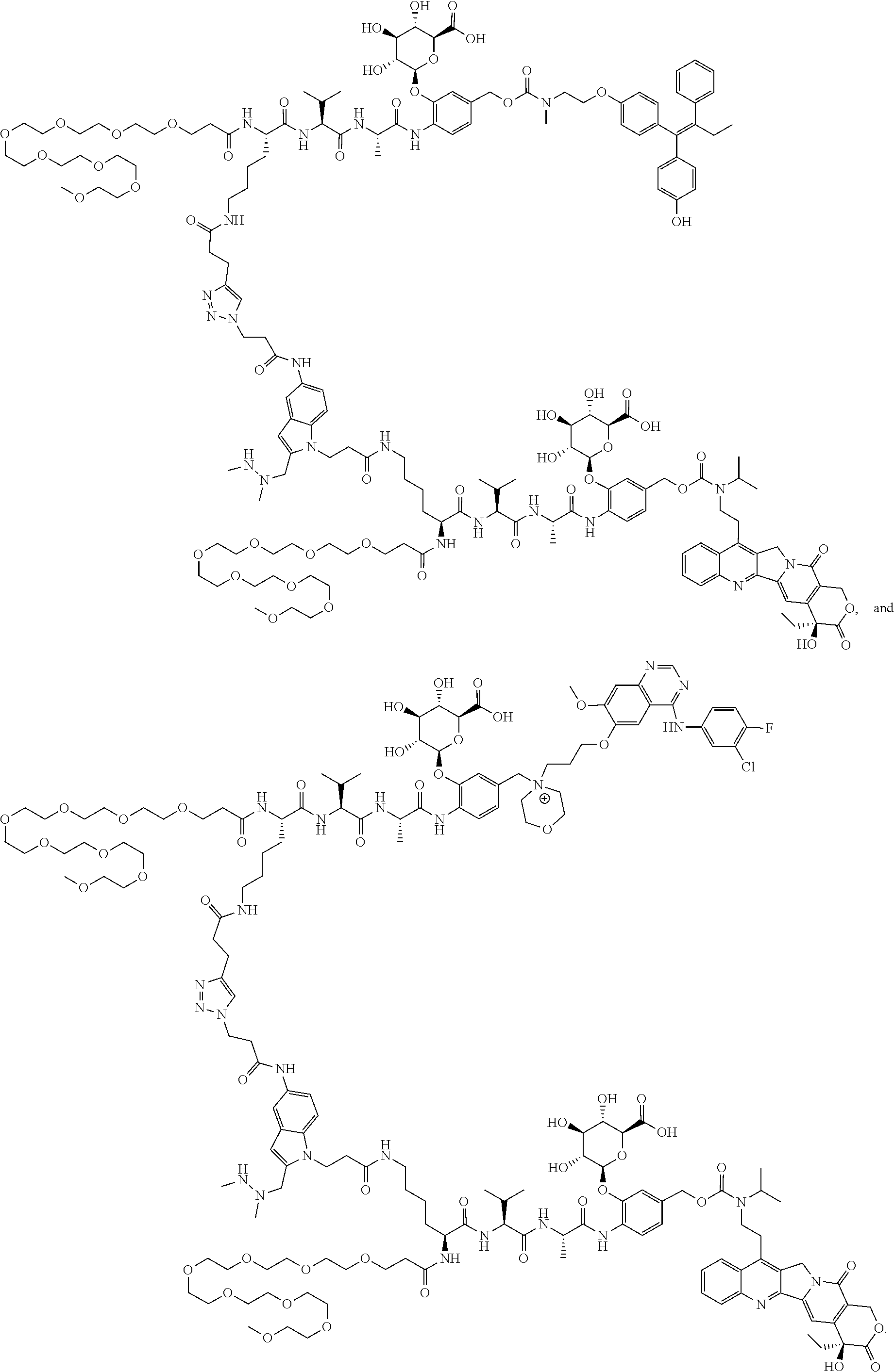

Any of the chemical entities, linkers and conjugation moieties set forth in the structures above may be adapted for use in the subject compounds and conjugates.

### Polypeptides and Antibodies

As noted above, a subject conjugate can comprise as substituent W³ a polypeptide (e.g., an antibody). The amino acid sequence of the polypeptide (antibody) can be modified to include a 2-formylglycine (fGly) residue. As used herein, amino acids may be referred to by their standard name, their standard three letter abbreviation and/or their standard one letter abbreviation, such as: Alanine or Ala or A; Cysteine or Cys or C; Aspartic acid or Asp or D; Glutamic acid or Glu or E; Phenylalanine or Phe or F; Glycine or Gly or G; Histidine or His or H; Isoleucine or Ile or I; Lysine or Lys or K; Leucine or Leu or L; Methionine or Met or M; Asparagine or Asn or N; Proline or Pro or P; Glutamine or Gln or Q; Arginine or Arg or R; Serine or Ser or S; Threonine or Thr or T; Valine or Val or V; Tryptophan or Trp or W; and Tyrosine or Tyr or Y.

In certain embodiments, the amino acid sequence of a polypeptide or an antibody is modified to include a sulfatase motif that contains a serine or cysteine residue that is capable of being converted (oxidized) to a 2-formylglycine (fGly) residue by action of a formylglycine generating enzyme (FGE) either in vivo (e.g., at the time of translation of an aldehyde tag-containing protein in a cell) or in vitro (e.g., by contacting an aldehyde tag-containing protein with an FGE in a cell-free system). Such sulfatase motifs may also be referred to herein as an FGE-modification site.

### Sulfatase Motifs

A minimal sulfatase motif of an aldehyde tag is usually 5 or 6 amino acid residues in length, usually no more than 6 amino acid residues in length. Sulfatase motifs provided in an Ig polypeptide are at least 5 or 6 amino acid residues, and can be, for example, from 5 to 16, 6-16, 5-15, 6-15, 5-14, 6-14, 5-13, 6-13, 5-12, 6-12, 5-11, 6-11, 5-10, 6-10, 5-9, 6-9, 5-8, or 6-8 amino acid residues in length, so as to define a sulfatase motif of less than 16, 15, 14, 13, 12, 11, 10, 9, 8, 7 or 6 amino acid residues in length.

In certain embodiments, polypeptides of interest include those where one or more amino acid residues, such as 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more amino acid residues have been inserted, deleted, substituted (replaced) relative to the native amino acid sequence to provide for a sequence of a sulfatase motif in the polypeptide. In certain embodiments, the polypeptide includes a modification (insertion, addition, deletion, and/or substitution/replacement) of less than 20, 19, 18, 17, 16, 15, 14, 13, 12, 11, 10, 9, 8, 7, 6, 5, 4, 3 or 2 amino acid residues of the amino acid sequence relative to the native amino acid sequence of the polypeptide. Where an amino acid sequence native to the polypeptide (e.g., antibody) contains one or more residues of the desired sulfatase motif, the total number of modifications of residues can be reduced, e.g., by site-specification modification (insertion, addition, deletion, substitution/replacement) of amino acid residues flanking the native amino acid residues to provide a sequence of the desired sulfatase motif. In certain embodiments, the extent of modification of the native amino acid sequence of the target antibody is minimized, so as to minimize the number of amino acid residues that are inserted, deleted, substituted (replaced), or added (e.g., to the N- or C-terminus). Minimizing the extent of amino acid sequence modification of the target antibody may minimize the impact such modifications may have upon antibody function and/or structure.

It should be noted that while aldehyde tags of particular interest are those comprising at least a minimal sulfatase motif (also referred to a “consensus sulfatase motif”), it will be readily appreciated that longer aldehyde tags are both contemplated and encompassed by the present disclosure and can find use in the compositions and methods of the present disclosure. Aldehyde tags can thus comprise a minimal sulfatase motif of 5 or 6 residues, or can be longer and comprise a minimal sulfatase motif which can be flanked at the N- and/or C-terminal sides of the motif by additional amino acid residues. Aldehyde tags of, for example, 5 or 6 amino acid residues are contemplated, as well as longer amino acid sequences of more than 5, 6, 7, 8, 9, 10, 11, 12, 13, 14, 15, 16, 17, 18, 19, 20 or more amino acid residues.

An aldehyde tag can be present at or near the C-terminus of an Ig heavy chain; e.g., an aldehyde tag can be present within 1, 2, 3, 4, 5, 6, 7, 8, 9, or 10 amino acids of the C-terminus of a native, wild-type Ig heavy chain. An aldehyde tag can be present within a CH1 domain of an Ig heavy chain. An aldehyde tag can be present within a CH₂ domain of an Ig heavy chain. An aldehyde tag can be present within a CH₃ domain of an Ig heavy chain. An aldehyde tag can be present in an Ig light chain constant region, e.g., in a kappa light chain constant region or a lambda light chain constant region.

In certain embodiments, the sulfatase motif used may be described by the formula:

X¹Z¹⁰X²Z²⁰X³Z³⁰ (I′)
where
Z¹⁰ is cysteine or serine (which can also be represented by (C/S));
Z²⁰ is either a proline or alanine residue (which can also be represented by (P/A));
Z³⁰ is a basic amino acid (e.g., arginine (R), and may be lysine (K) or histidine (H), e.g., lysine), or an aliphatic amino acid (alanine (A), glycine (G), leucine (L), valine (V), isoleucine (I), or proline (P), e.g., A, G, L, V, or I;
X¹ is present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M, S or V, with the proviso that when the sulfatase motif is at the N-terminus of the target polypeptide, X¹ is present; and
X² and X³ independently can be any amino acid, though usually an aliphatic amino acid, a polar, uncharged amino acid, or a sulfur containing amino acid (i.e., other than an aromatic amino acid or a charged amino acid), e.g., S, T, A, V, G or C, e.g., S, T, A, V or G.

The amino acid sequence of an antibody heavy and/or light chain can be modified to provide a sequence of at least 5 amino acids of the formula X¹Z¹⁰X²Z²⁰X³Z³⁰, where
Z¹⁰ is cysteine or serine;
Z²⁰ is a proline or alanine residue;
Z³⁰ is an aliphatic amino acid or a basic amino acid;
X¹ is present or absent and, when present, is any amino acid, with the proviso that when the heterologous sulfatase motif is at an N-terminus of the polypeptide, X¹ is present;
X² and X³ are each independently any amino acid.

The sulfatase motif is generally selected so as to be capable of conversion by a selected FGE, e.g., an FGE present in a host cell in which the aldehyde tagged polypeptide is expressed or an FGE which is to be contacted with the aldehyde tagged polypeptide in a cell-free in vitro method.

For example, where the FGE is a eukaryotic FGE (e.g., a mammalian FGE, including a human FGE), the sulfatase motif can be of the formula:

X¹CX²PX³Z³⁰ (I″)
where
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, S or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G, or C, e.g., S, T, A, V or G; and
Z³⁰ is a basic amino acid (e.g., arginine (R), and may be lysine (K) or histidine (H), e.g., lysine), or an aliphatic amino acid (alanine (A), glycine (G), leucine (L), valine (V), isoleucine (I), or proline (P), e.g., A, G, L, V, or I.

Specific examples of sulfatase motifs include LCTPSR (SEQ ID NO://), MCTPSR (SEQ ID NO://), VCTPSR (SEQ ID NO://), LCSPSR (SEQ ID NO://), LCAPSR (SEQ ID NO://), LCVPSR (SEQ ID NO://), LCGPSR (SEQ ID NO://), ICTPAR (SEQ ID NO://), LCTPSK (SEQ ID NO://), MCTPSK (SEQ ID NO://), VCTPSK (SEQ ID NO://), LCSPSK (SEQ ID NO://), LCAPSK (SEQ ID NO://), LCVPSK (SEQ ID NO://), LCGPSK (SEQ ID NO://), LCTPSA (SEQ ID NO://), ICTPAA (SEQ ID NO://), MCTPSA (SEQ ID NO://), VCTPSA (SEQ ID NO://), LCSPSA (SEQ ID NO://), LCAPSA (SEQ ID NO://), LCVPSA (SEQ ID NO://), and LCGPSA (SEQ ID NO://).

fGly-Containing Sequences

Upon action of FGE on the antibody heavy and/or light chain, the serine or the cysteine in the sulfatase motif is modified to fGly. Thus, the fGly-containing sulfatase motif can be of the formula:

X¹(fGly)X²Z²⁰X³Z³⁰ (I′″)
where
fGly is the formylglycine residue;
Z²⁰ is either a proline or alanine residue (which can also be represented by (P/A)); Z³⁰ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target polypeptide, X¹ is present; and
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G.

As described above, to produce the conjugate, the polypeptide containing the fGly residue may be conjugated to a drug or active agent by reaction of the fGly with a reactive moiety (e.g., a hydrazinyl-indolyl or a hydrazinyl-pyrrolo-pyridinyl conjugation moiety, as described above) of a linker attached to the drug or active agent to produce an fGly′-containing sulfatase motif. As used herein, the term fGly′ refers to the amino acid residue of the sulfatase motif that is coupled to the drug or active agent through a linker (e.g., a branched linker) as described herein. Thus, the fGly′-containing sulfatase motif can be of the formula:

X¹(fGly′)X²Z²⁰X³Z³⁰ (II)
where
   fGly′ is the amino acid residue coupled to the drug or active agent through a linker (e.g., a branched linker) as described herein;
   Z²⁰ is either a proline or alanine residue (which can also be represented by (P/A));
   Z³⁰ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
   X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target polypeptide, X¹ is present; and
   X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G.

### Site of Modification

As noted above, the amino acid sequence of an antibody is modified to include a sulfatase motif that contains a serine or cysteine residue that is capable of being converted (oxidized) to an fGly residue by action of an FGE either in vivo (e.g., at the time of translation of an aldehyde tag-containing protein in a cell) or in vitro (e.g., by contacting an aldehyde tag-containing protein with an FGE in a cell-free system). The antibody used to generate a conjugate of the present disclosure include at least an Ig constant region, e.g., an Ig heavy chain constant region (e.g., at least a CH1 domain; at least a CH1 and a CH2 domain; a CH1, a CH2, and a CH3 domain; or a CH1, a CH2, a CH3, and a CH4 domain), or an Ig light chain constant region. Such Ig polypeptides are referred to herein as “target Ig polypeptides” or “target antibodies”.

The site in an antibody into which a sulfatase motif is introduced can be any convenient site. As noted above, in some instances, the extent of modification of the native amino acid sequence of the target polypeptide is minimized, so as to minimize the number of amino acid residues that are inserted, deleted, substituted (replaced), and/or added (e.g., to the N- or C-terminus). Minimizing the extent of amino acid sequence modification of the target antibody may minimize the impact such modifications may have upon antibody function and/or structure.

An antibody heavy chain constant region can include Ig constant regions of any heavy chain isotype, non-naturally occurring Ig heavy chain constant regions (including consensus Ig heavy chain constant regions). An Ig constant region amino acid sequence can be modified to include an aldehyde tag, where the aldehyde tag is present in or adjacent a solvent-accessible loop region of the Ig constant region. An Ig constant region amino acid sequence can be modified by insertion and/or substitution of 1, 2, 3, 4, 5, 6, 7, 8, 9, 10, 11, 12, 13, 14, 15, or 16 amino acids, or more than 16 amino acids, to provide an amino acid sequence of a sulfatase motif as described above.

In some cases, an aldehyde-tagged antibody comprises an aldehyde-tagged Ig heavy chain constant region (e.g., at least a CH1 domain; at least a CH1 and a CH2 domain; a CH1, a CH2, and a CH3 domain; or a CH1, a CH2, a CH3, and a CH4 domain). The aldehyde-tagged Ig heavy chain constant region can include heavy chain constant region sequences of an IgA, IgM, IgD, IgE, IgG1, IgG2, IgG3, or IgG4 isotype heavy chain or any allotypic variant of same, e.g., human heavy chain constant region sequences or mouse heavy chain constant region sequences, a hybrid heavy chain constant region, a synthetic heavy chain constant region, or a consensus heavy chain constant region sequence, etc., modified to include at least one sulfatase motif that can be modified by an FGE to generate an fGly-modified Ig polypeptide. Allotypic variants of Ig heavy chains are known in the art. See, e.g., Jefferis and Lefranc (2009) MAbs 1:4.

In some cases, an aldehyde-tagged antibody comprises an aldehyde-tagged Ig light chain constant region. The aldehyde-tagged Ig light chain constant region can include constant region sequences of a kappa light chain, a lambda light chain, e.g., human kappa or lambda light chain constant regions, a hybrid light chain constant region, a synthetic light chain constant region, or a consensus light chain constant region sequence, etc., modified to include at least one sulfatase motif that can be modified by an FGE to generate an fGly-modified antibody. Exemplary constant regions include human gamma 1 and gamma 3 regions. With the exception of the sulfatase motif, a constant region may have a wild-type amino acid sequence, or it may have an amino acid sequence that is at least 70% identical (e.g., at least 80%, at least 90% or at least 95% identical) to a wild type amino acid sequence.

In some embodiments the sulfatase motif is at a position other than, or in addition to, the C-terminus of the Ig polypeptide heavy chain. As noted above, an isolated aldehyde-tagged antibody can comprise a heavy chain constant region amino acid sequence modified to include a sulfatase motif as described above, where the sulfatase motif is in or adjacent a surface-accessible loop region of the antibody heavy chain constant region.

A sulfatase motif can be provided within or adjacent one or more of these amino acid sequences of such modification sites of an Ig heavy chain. For example, an Ig heavy chain polypeptide amino acid sequence can be modified (e.g., where the modification includes one or more amino acid residue insertions, deletions, and/or substitutions) at one or more of these amino acid sequences to provide a sulfatase motif adjacent and N-terminal and/or adjacent and C-terminal to these modification sites. Alternatively or in addition, an Ig heavy chain polypeptide amino acid sequence can be modified (e.g., where the modification includes one or more amino acid residue insertions, deletions, and/or substitutions) at one or more of these amino acid sequences to provide a sulfatase motif between any two residues of the Ig heavy chain modifications sites. In some embodiments, an Ig heavy chain polypeptide amino acid sequence may be modified to include two motifs, which may be adjacent to one another, or which may be separated by one, two, three, four or more (e.g., from about 1 to about 25, from about 25 to about 50, or from about 50 to about 100, or more, amino acids. Alternatively or in addition, where a native amino acid sequence provides for one or more amino acid residues of a sulfatase motif sequence, selected amino acid residues of the modification sites of an Ig heavy chain polypeptide amino acid sequence can be modified (e.g., where the modification includes one or more amino acid residue insertions, deletions, and/or substitutions) so as to provide a sulfatase motif at the modification site.

An antibody used in an antibody-drug conjugate of the present disclosure can have any of a variety of antigen-binding specificities, including but not limited to, e.g., an antigen present on a cancer cell; an antigen present on an autoimmune cell; an antigen present on a pathogenic microorganism; an antigen present on a virus-infected cell (e.g., a human immunodeficiency virus-infected cell); an antigen present on a diseased cell; and the like. For example, an antibody conjugate can bind an antigen, where the antigen is present on the surface of the cell. An antibody conjugate of the present disclosure can bind antigen with a suitable binding affinity, e.g., from 5×10⁻⁶ M to 10⁻⁷ M, from 10⁻⁷ M to 5×10⁻⁷ M, from 5×10⁻⁷ M to 10⁻⁸ M, from 10⁻⁸ M to 5×10⁻⁸ M, from 5×10⁻⁸ M to 10⁻⁹ M, or a binding affinity greater than 10⁻⁹ M.

As non-limiting examples, a subject antibody conjugate can bind an antigen present on a cancer cell (e.g., a tumor-specific antigen; an antigen that is over-expressed on a cancer cell; etc.), and the conjugated moiety can be a drug, such as a cytotoxic compound (e.g., a cytotoxic small molecule, a cytotoxic synthetic peptide, etc.). For example, a subject antibody conjugate can be specific for an antigen on a cancer cell, where the conjugated moiety is a drug, such as a cytotoxic compound (e.g., a cytotoxic small molecule, a cytotoxic synthetic peptide, etc.).

As further non-limiting examples, a subject antibody conjugate can bind an antigen present on a cell infected with a virus (e.g., where the antigen is encoded by the virus; where the antigen is expressed on a cell type that is infected by a virus; etc.), and the conjugated moiety can be a drug, such as a viral fusion inhibitor. For example, a subject antibody conjugate can bind an antigen present on a cell infected with a virus, and the conjugated moiety can be a drug, such as a viral fusion inhibitor.

### Drugs for Conjugation to a Polypeptide

As noted above, a conjugate or a compound of the present disclosure can include as substituents W¹ and W² a drug or active agent. Any of a number of drugs are suitable for use, or can be modified to be rendered suitable for use, as a reactive partner to conjugate to an antibody. Examples of drugs include small molecule drugs and peptide drugs.

“Small molecule drug” as used herein refers to a compound, e.g., an organic compound, which exhibits a pharmaceutical activity of interest and which is generally of a molecular weight of 800 Da or less, or 2000 Da or less, but can encompass molecules of up to 5 kDa and can be as large as 10 kDa. A small inorganic molecule refers to a molecule containing no carbon atoms, while a small organic molecule refers to a compound containing at least one carbon atom.

For example, the drug or active agent can be a topoisomerase inhibitor (e.g., a topoisomerase I inhibitor), such as a camptothecine, or an analog or derivative thereof, or a pharmaceutically active camptothecine moiety and/or a portion thereof. A topoisomerase inhibitor (e.g., camptothecine, or analog or derivative thereof) conjugated to the polypeptide can be any of a variety of topoisomerase inhibitors, for example camptothecine or camptothecine moieties such as, but not limited to, camptothecine and analogs and derivatives thereof as described herein. Examples of drugs that find use in the conjugates and compounds described herein include, but are not limited to, a topoisomerase inhibitor, for example camptothecine or a camptothecine derivative, such as SN-38, Belotecan, Exatecan, 9-aminocamptothecin (9-AC), topotecan, des-Me-topotecan, derivatives thereof, and the like. Additional examples of topoisomerase inhibitors that find use in the present disclosure are described in PCT/US2022/012325, the disclosure of which is incorporated herein by reference.

In other embodiments, the drug or active agent can be a maytansine. “Maytansine”, “maytansine moiety”, “maytansine active agent moiety” and “maytansinoid” refer to a maytansine and analogs and derivatives thereof, and pharmaceutically active maytansine moieties and/or portions thereof. A maytansine conjugated to the polypeptide can be any of a variety of maytansinoid moieties such as, but not limited to, maytansine and analogs and derivatives thereof as described herein (e.g., deacylmaytansine).

In other instances, the drug or active agent can be an auristatin, or an analog or derivative thereof, or a pharmaceutically active auristatin moiety and/or a portion thereof. An auristatin conjugated to the polypeptide can be any of a variety of auristatin moieties such as, but not limited to, an auristatin and analogs and derivatives thereof as described herein. Examples of drugs that find use in the conjugates and compounds described herein include, but are not limited to an auristatin or an auristatin derivative, such as monomethyl auristatin D (MMAD), monomethyl auristatin E (MMAE), monomethyl auristatin F (MMAF), derivatives thereof, and the like.

In other cases, the drug or active agent can be a duocarmycin, or an analog or derivative thereof, or a pharmaceutically active duocarmycin moiety and/or a portion thereof. A duocarmycin conjugated to the polypeptide can be any of a variety of duocarmycin moieties such as, but not limited to, a duocarmycin and analogs and derivatives thereof as described herein. Examples of drugs that find use in the conjugates and compounds described herein include, but are not limited to a duocarmycin or a duocarmycin derivative, such as duocarmycin A, duocarmycin B1, duocarmycin B2, duocarmycin C₁, duocarmycin C₂, duocarmycin D, duocarmycin SA, and CC-1065, derivatives thereof, and the like. In some embodiments, the duocarmycin is a duocarmycin analog, such as, but not limited to, adozelesin, bizelesin, or carzelesin.

In certain embodiments, the drug is selected from a cytotoxin, a kinase inhibitor, a selective estrogen receptor modulator, an immunostimulatory agent, a toll-like receptor (TLR) agonist, an oligonucleotide, an aptamer, a cytokine, a steroid, and a peptide.

For example, a cytotoxin can include any compound that leads to cell death (e.g., necrosis or apoptosis) or a decrease in cell viability.

Kinase inhibitors can include, but are not limited to, Adavosertib, Afatinib, Axitinib, Bosutinib, Cetuximab, Cobimetinib, Crizotinib, Cabozantinib, Dacomitinib, Dasatinib, Entrectinib, Erdafitinib, Erlotinib, Fostamatinib, Gefitinib, Ibrutinib, Imatinib, Lapatinib, Lenvatinib, Mubritinib, Nilotinib, Pazopanib, Pegaptanib, Ruxolitinib, Sorafenib, Sunitinib, Tucatinib, Vandetanib, Vemurafenib, and the like.

For example, selective estrogen receptor modulators include, but are not limited to, Endoxifen, Tamoxifen, Afimoxifene, Toremifene, and the like.

Immunostimulatory agents can include, but are not limited to, vaccines (e.g., bacterial or viral vaccines), colony stimulating factors, interferons, interleukins, and the like. TLR agonists include, but are not limited to, imiquimod, resiquimod, and the like.

Oligonucleotide dugs include, but are not limited to, fomivirsen, pegaptanib, mipomersen, eteplirsen, defibrotide, nusinersen, golodirsen, viltolarsen, volanesorsen, inotersen, tofersen, tominersen, and the like.

Aptamer drugs include, but are not limited to, pegaptanib, AS1411, REG1, ARC1779, NU172, ARC1905, E10030, NOX-A12, NOX-E36, and the like.

Cytokines include, but are not limited to, Albinterferon Alfa-2B, Aldesleukin, ALT-801, Anakinra, Ancestim, Avotermin, Balugrastim, Bempegaldesleukin, Binetrakin, Cintredekin Besudotox, CTCE-0214, Darbepoetin alfa, Denileukin diftitox, Dulanermin, Edodekin alfa, Emfilermin, Epoetin delta, Erythropoietin, Human interleukin-2, Interferon alfa, Interferon alfa-2c, Interferon alfa-n1, Interferon alfa-n3, Interferon alfacon-1, Interferon beta-1a, Interferon beta-1b, Interferon gamma-1b, Interferon Kappa, Interleukin-1 alpha, Interleukin-10, Interleukin-7, Lenograstim, Leridistim, Lipegfilgrastim, Lorukafusp alfa, Maxy-G34, Methoxy polyethylene glycol-epoetin beta, Molgramostim, Muplestim, Nagrestipen, Oprelvekin, Pegfilgrastim, Pegilodecakin, Peginterferon alfa-2a, Peginterferon alfa-2b, Peginterferon beta-1a, Peginterferon lambda-1a, Recombinant CD40-ligand, Regramostim, Romiplostim, Sargramostim, Thrombopoietin, Tucotuzumab celmoleukin, Viral Macrophage-Inflammatory Protein, and the like.

Steroid drugs include, but are not limited to, prednisolone, betamethasone, dexamethasone, hydrocortisone, methylprednisolone, deflazacort, and the like.

“Peptide drug” as used herein refers to amino-acid containing polymeric compounds, and is meant to encompass naturally-occurring and non-naturally-occurring peptides, oligopeptides, cyclic peptides, polypeptides, and proteins, as well as peptide mimetics. The peptide drugs may be obtained by chemical synthesis or be produced from a genetically encoded source (e.g., recombinant source). Peptide drugs can range in molecular weight, and can be from 200 Da to 10 kDa or greater in molecular weight. Suitable peptides include, but are not limited to, cytotoxic peptides; angiogenic peptides; anti-angiogenic peptides; peptides that activate B cells; peptides that activate T cells; anti-viral peptides; peptides that inhibit viral fusion; peptides that increase production of one or more lymphocyte populations; anti-microbial peptides; growth factors; growth hormone-releasing factors; vasoactive peptides; anti-inflammatory peptides; peptides that regulate glucose metabolism; an anti-thrombotic peptide; an anti-nociceptive peptide; a vasodilator peptide; a platelet aggregation inhibitor; an analgesic; and the like.

Additional examples of drugs that find use in the conjugates and compounds described herein include, but are not limited to Tubulysin M, Calicheamicin, a STAT3 inhibitor, alpha-Amanitin, an aurora kinase inhibitor, belotecan, and an anthracycline.

Other examples of drugs include small molecule drugs, such as a cancer chemotherapeutic agent. For example, where the polypeptide is an antibody (or fragment thereof) that has specificity for a tumor cell, the antibody can be produced as described herein to include a modified amino acid, which can be subsequently conjugated to a cancer chemotherapeutic agent. Cancer chemotherapeutic agents include non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones. Peptidic compounds can also be used.

Suitable cancer chemotherapeutic agents include dolastatin and active analogs and derivatives thereof; and auristatin and active analogs and derivatives thereof (e.g., Monomethyl auristatin D (MMAD), monomethyl auristatin E (MMAE), monomethyl auristatin F (MMAF), and the like). See, e.g., WO 96/33212, WO 96/14856, and U.S. Pat. No. 6,323,315. For example, dolastatin 10 or auristatin PE can be included in an antibody-drug conjugate of the present disclosure. Suitable cancer chemotherapeutic agents also include maytansinoids and active analogs and derivatives thereof (see, e.g., EP 1391213; and Liu et al (1996) Proc. Natl. Acad. Sci. USA 93:8618-8623); duocarmycins and active analogs and derivatives thereof (e.g., including the synthetic analogues, KW-2189 and CB 1-TM1); and benzodiazepines and active analogs and derivatives thereof (e.g., pyrrolobenzodiazepine (PBD).

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation; therefore compounds that bind to the estrogen receptor are used to block this activity. Corticosteroids may inhibit T cell proliferation.

Other suitable chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

Taxanes are suitable for use. “Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Biological response modifiers suitable for use include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) IFN-α; (7) IFN-7; (8) colony-stimulating factors; and (9) inhibitors of angiogenesis.

Examples of drugs include small molecule drugs, such as a cancer chemotherapeutic agent. For example, where the polypeptide is an antibody (or fragment thereof) that has specificity for a tumor cell, the antibody can be produced as described herein to include a modified amino acid, which can be subsequently conjugated to a cancer chemotherapeutic agent, such as a microtubule affecting agent. In certain embodiments, the drug is a microtubule affecting agent that has antiproliferative activity, such as a maytansinoid.

Embodiments of the present disclosure include conjugates where an antibody is conjugated to two or more drug moieties, such as 3 drug moieties, 4 drug moieties, 5 drug moieties, 6 drug moieties, 7 drug moieties, 8 drug moieties, 9 drug moieties, 10 drug moieties, 11 drug moieties, 12 drug moieties, 13 drug moieties, 14 drug moieties, 15 drug moieties, 16 drug moieties, 17 drug moieties, 18 drug moieties, 19 drug moieties, or 20 or more drug moieties. The drug moieties may be conjugated to the antibody at one or more sites in the antibody, as described herein. In certain embodiments, the conjugates have an average drug-to-antibody ratio (DAR) (molar ratio) in the range of from 0.1 to 20, or from 0.5 to 20, or from 1 to 20, such as from 1 to 19, or from 1 to 18, or from 1 to 17, or from 1 to 16, or from 1 to 15, or from 1 to 14, or from 1 to 13, or from 1 to 12, or from 1 to 11, or from 1 to 10, or from 1 to 9, or from 1 to 8, or from 1 to 7, or from 1 to 6, or from 1 to 5, or from 1 to 4, or from 1 to 3, or from 1 to 2. In certain embodiments, the conjugates have an average DAR from 1 to 10, such as 1, 2, 3, 4, 5, 6, 7, 8, 9, or 10. In certain embodiments, the conjugates have an average DAR of 1 to 10. In certain embodiments, the conjugates have an average DAR of 1 to 5 (e.g., 4). In certain embodiments, the conjugates have an average DAR of 5 to 10 (e.g., 8). By average is meant the arithmetic mean.

In certain embodiments, the two drugs or active agents attached to the branched linker are the same drug or active agent. For example, a first branch of a branched linker may be attached to a drug or an active agent and a second branch of the branched linker may be attached to the same drug or the same active agent as the first branch. In other embodiments, the two drugs or active agents attached to the branched linker are different drugs or active agents. For example, a first branch of a branched linker may be attached to a first drug or a first active agent and a second branch of the branched linker may be attached to a second drug or a second active agent different from the first drug or the first active agent attached to the first branch.

In some embodiments, where two different drugs or active agents are attached to the branched linker, the drugs or active agents may be selected from drugs and active agents that have a synergistic therapeutic effect. For example, in some instances, the use of two different drugs or active agents attached to the branched linker may provide a lower therapeutically effective concentration at which both payloads act, thereby increasing overall potency of the ADC.

In some embodiments, where two different drugs or active agents are attached to the branched linker, the drugs or active agents may be selected from drugs and active agents that provide an enhanced therapeutic benefit as compared to the use of the drugs or active agents separately, For example, the drugs or active agents may provide an increased effect on drug delivery of the ADC (e.g., some payloads, such as the iRGD peptide, can increase extravasation into tissues and augment tumor penetration).

In some embodiments, where two different drugs or active agents are attached to the branched linker, the drugs or active agents may be selected from drugs and active agents that use different mechanisms of action. In some cases, this may provide a decrease in tumor drug resistance by targeting multiple pathways. Examples of payload combinations can include, but are not limited to, cytotoxic drugs, immunomodulatory molecules to activate or inhibit immune cell populations, cytokines, hormones, chelating agents loaded with radioisotopes, and the like.

In some embodiments, where two different drugs or active agents are attached to the branched linker, the two different drugs or active agents are a topoisomerase inhibitor (e.g., belotecan) as described herein and an auristatin (e.g., MMAE) as described herein. In some embodiments, where two different drugs or active agents are attached to the branched linker, the two different drugs or active agents are a topoisomerase inhibitor (e.g., belotecan) as described herein and an iRGD peptide as described herein. In some embodiments, where two different drugs or active agents are attached to the branched linker, the two different drugs or active agents are an auristatin (e.g., MMAE) as described herein and an iRGD peptide as described herein. In some embodiments, where two different drugs or active agents are attached to the branched linker, the two different drugs or active agents are an auristatin (e.g., MMAE) as described herein and a kinase inhibitor (e.g., Sorafenib, Lapatinib, Gefitinib, and the like) as described herein. In some embodiments, where two different drugs or active agents are attached to the branched linker, the two different drugs or active agents are a topoisomerase inhibitor (e.g., belotecan) as described herein and a kinase inhibitor (e.g., Sorafenib, Lapatinib, Gefitinib, and the like) as described herein. In some embodiments, where two different drugs or active agents are attached to the branched linker, the two different drugs or active agents are an auristatin (e.g., MMAE) as described herein and a selective estrogen receptor modulator (e.g., Endoxifen) as described herein. In some embodiments, where two different drugs or active agents are attached to the branched linker, the two different drugs or active agents are a topoisomerase inhibitor (e.g., belotecan) as described herein and a selective estrogen receptor modulator (e.g., Endoxifen) as described herein.

Drugs to be conjugated to a polypeptide may be modified to incorporate a reactive partner for reaction with the polypeptide. Where the drug is a peptide drug, the reactive moiety (e.g., aminooxy or hydrazide can be positioned at an N-terminal region, the N-terminus, a C-terminal region, the C-terminus, or at a position internal to the peptide. For example, an example of a method involves synthesizing a peptide drug having an aminooxy group. In this example, the peptide is synthesized from a Boc-protected precursor. An amino group of a peptide can react with a compound comprising a carboxylic acid group and oxy-N-Boc group. As an example, the amino group of the peptide reacts with 3-(2,5-dioxopyrrolidin-1-yloxy)propanoic acid. Other variations on the compound comprising a carboxylic acid group and oxy-N-protecting group can include different number of carbons in the alkylene linker and substituents on the alkylene linker. The reaction between the amino group of the peptide and the compound comprising a carboxylic acid group and oxy-N-protecting group occurs through standard peptide coupling chemistry. Examples of peptide coupling reagents that can be used include, but not limited to, DCC (dicyclohexylcarbodiimide), DIC (diisopropylcarbodiimide), di-p-toluoylcarbodiimide, BDP (1-benzotriazole diethylphosphate-1-cyclohexyl-3-(2-morpholinylethyl)carbodiimide), EDC (1-(3-dimethylaminopropyl-3-ethyl-carbodiimide hydrochloride), cyanuric fluoride, cyanuric chloride, TFFH (tetramethyl fluoroformamidinium hexafluorophosphosphate), DPPA (diphenylphosphorazidate), BOP (benzotriazol-1-yloxytris(dimethylamino)phosphonium hexafluorophosphate), HBTU (O-benzotriazol-1-yl-N,N,N′,N′-tetramethyluronium hexafluorophosphate), TBTU (O-benzotriazol-1-yl-N,N,N′,N′-tetramethyluronium tetrafluoroborate), TSTU (O-(N-succinimidyl)-N,N,N′,N′-tetramethyluronium tetrafluoroborate), HATU (N-[(dimethylamino)-1-H-1,2,3-triazolo[4,5,6]-pyridin-1-ylmethylene]-N-methylmethanaminium hexafluorophosphate N-oxide), BOP-Cl (bis(2-oxo-3-oxazolidinyl)phosphinic chloride), PyBOP ((1-H-1,2,3-benzotriazol-1-yloxy)-tris(pyrrolidino)phosphonium tetrafluorophopsphate), BrOP (bromotris(dimethylamino)phosphonium hexafluorophosphate), DEPBT (3-(diethoxyphosphoryloxy)-1,2,3-benzotriazin-4(3H)-one) PyBrOP (bromotris(pyrrolidino)phosphonium hexafluorophosphate). As a non-limiting example, HOBt and DIC can be used as peptide coupling reagents.

Deprotection to expose the amino-oxy functionality is performed on the peptide comprising an N-protecting group. Deprotection of the N-oxysuccinimide group, for example, occurs according to standard deprotection conditions for a cyclic amide group. Deprotecting conditions can be found in Greene and Wuts, Protective Groups in Organic Chemistry, 3rd Ed., 1999, John Wiley & Sons, NY and Harrison et al. Certain deprotection conditions include a hydrazine reagent, amino reagent, or sodium borohydride. Deprotection of a Boc protecting group can occur with TFA. Other reagents for deprotection include, but are not limited to, hydrazine, methylhydrazine, phenylhydrazine, sodium borohydride, and methylamine. The product and intermediates can be purified by conventional means, such as HPLC purification.

The ordinarily skilled artisan will appreciate that factors such as pH and steric hindrance (i.e., the accessibility of the amino acid residue to reaction with a reactive partner of interest) are of importance, Modifying reaction conditions to provide for optimal conjugation conditions is well within the skill of the ordinary artisan, and is routine in the art. Where conjugation is conducted with a polypeptide present in or on a living cell, the conditions are selected so as to be physiologically compatible. For example, the pH can be dropped temporarily for a time sufficient to allow for the reaction to occur but within a period tolerated by the cell (e.g., from about 30 min to 1 hour). Physiological conditions for conducting modification of polypeptides on a cell surface can be similar to those used in a ketone-azide reaction in modification of cells bearing cell-surface azides (see, e.g., U.S. Pat. No. 6,570,040).

Small molecule compounds containing, or modified to contain, an X-nucleophilic group that serves as a reactive partner with a compound or conjugate disclosed herein are also contemplated for use as drugs in the polypeptide-drug conjugates of the present disclosure. General methods are known in the art for chemical synthetic schemes and conditions useful for synthesizing a compound of interest (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

### Formulations

The conjugates of the present disclosure can be formulated in a variety of different ways. In general, where the conjugate is an antibody-drug conjugate, the conjugate is formulated in a manner compatible with the drug, the antibody, the condition to be treated, and the route of administration to be used.

In some embodiments, provided is a pharmaceutical composition that includes any of the conjugates of the present disclosure and a pharmaceutically-acceptable excipient.

The conjugate (e.g., antibody-drug conjugate) can be provided in any suitable form, e.g., in the form of a pharmaceutically acceptable salt, and can be formulated for any suitable route of administration, e.g., oral, topical or parenteral administration. Where the conjugate is provided as a liquid injectable (such as in those embodiments where they are administered intravenously or directly into a tissue), the conjugate can be provided as a ready-to-use dosage form, or as a reconstitutable storage-stable powder or liquid composed of pharmaceutically acceptable carriers and excipients.

Methods for formulating conjugates can be adapted from those readily available. For example, conjugates can be provided in a pharmaceutical composition comprising a therapeutically effective amount of a conjugate and a pharmaceutically acceptable carrier (e.g., saline). The pharmaceutical composition may optionally include other additives (e.g., buffers, stabilizers, preservatives, and the like). In some embodiments, the formulations are suitable for administration to a mammal, such as those that are suitable for administration to a human.

### Methods of Treatment

The antibody-drug conjugates of the present disclosure find use in treatment of a condition or disease in a subject that is amenable to treatment by administration of the parent drug (i.e., the drug prior to conjugation to the antibody).

In some embodiments, provided are methods that include administering to a subject an effective amount (e.g., a therapeutically effective amount) of any of the conjugates of the present disclosure.

In certain aspects, provided are methods of delivering a drug to a target site in a subject, the method including administering to the subject a pharmaceutical composition including any of the conjugates of the present disclosure, where the administering is effective to release a therapeutically effective amount of the drug from the conjugate at the target site in the subject. For example, as described herein, antibody-drug conjugates of the present disclosure can include a cleavable linker, such as an enzymatically cleavable linker that includes a first enzymatically cleavable moiety and a second enzymatically cleavable moiety. In some instances, the cleavable linker can be cleaved under appropriate conditions to separate or release the drug from the antibody at a desired target site of action for the drug. For example, the second cleavable linker, which protects the first cleavable linker from cleavage, may be cleaved in order to allow the first cleavable moiety to be cleaved, which results in cleavage of the cleavable linker into two or more portions, thus releasing the drug from the antibody-drug conjugate at a desired site of action.

In certain embodiments, the first cleavable moiety can be an enzymatically cleavable moiety. In some instances, the enzyme that facilitates cleavage of the first cleavable moiety is an enzyme that is administered to the subject to be treated (i.e., exogenous to the subject to be treated). For example, a first enzyme can be administered before, concurrently with, or after administration of an antibody-drug conjugate described herein.

In certain embodiments, the second cleavable moiety can be an enzymatically cleavable moiety. In some instances, the enzyme that facilitates cleavage of the second cleavable moiety is an enzyme that is administered to the subject to be treated (i.e., exogenous to the subject to be treated). For example, a second enzyme can be administered before, concurrently with, or after administration of an antibody-drug conjugate described herein. In certain embodiments, the first enzyme and the second enzyme are different enzymes.

In other instances, the first enzyme that facilitates cleavage of the first cleavable moiety is an enzyme that is present in the subject to be treated (i.e., endogenous to the subject to be treated). For instance, the first enzyme may be present at the desired site of action for the drug of the antibody-drug conjugate. The antibody of the antibody-drug conjugate may be specifically targeted to a desired site of action (e.g., may specifically bind to an antigen present at a desired site of action), where the desired site of action also includes the presence of the first enzyme. In some instances, the first enzyme is present in an overabundance at the desired site of action as compared to other areas in the body of the subject to be treated. For example, the first enzyme may be overexpressed at the desired site of action as compared to other areas in the body of the subject to be treated. In some instances, the first enzyme is present in an overabundance at the desired site of action due to localization of the first enzyme at a particular area or location. For instance, the first enzyme may be associated with a certain structure within the desired site of action, such as lysosomes. In some cases, the first enzyme is present in an overabundance in lysosomes as compared to other areas in the body of the subject. In some embodiments, the lysosomes that include the first enzyme, are found at a desired site of action for the drug of the antibody-drug conjugate, such as the site of a cancer or tumor that is to be treated with the drug. In certain embodiments, the first enzyme is an esterase.

In certain embodiments, the second enzyme that facilitates cleavage of the second cleavable moiety is an enzyme that is present in the subject to be treated (i.e., endogenous to the subject to be treated). For instance, the second enzyme may be present at the desired site of action for the drug of the antibody-drug conjugate. The antibody of the antibody-drug conjugate may be specifically targeted to a desired site of action (e.g., may specifically bind to an antigen present at a desired site of action), where the desired site of action also includes the presence of the second enzyme. In some instances, the second enzyme is present in an overabundance at the desired site of action as compared to other areas in the body of the subject to be treated. For example, the second enzyme may be overexpressed at the desired site of action as compared to other areas in the body of the subject to be treated. In some instances, the second enzyme is present in an overabundance at the desired site of action due to localization of the second enzyme at a particular area or location. For instance, the second enzyme may be associated with a certain structure within the desired site of action, such as lysosomes. In some cases, the second enzyme is present in an overabundance in lysosomes as compared to other areas in the body of the subject. In some embodiments, the lysosomes that include the second enzyme, are found at a desired site of action for the drug of the antibody-drug conjugate, such as the site of a cancer or tumor that is to be treated with the drug. In certain embodiments, the second enzyme is a glucuronidase, a galactosidase, a glucosidase, a mannosidase, a fucosidase, and the like.

Any suitable enzymes can be used for cleavage of the first cleavable moiety and the second cleavable moiety of the antibody-drug conjugates described herein. Other enzymes may also be suitable for use in cleavage of the first cleavable moiety and the second cleavable moiety of the antibody-drug conjugates described herein, such as but not limited to, enzymes from other vertebrates (e.g., primates, mice, rats, cats, pigs, quails, goats, dogs, etc.).

In certain embodiments, the antibody-drug conjugate is substantially stable under standard conditions. By substantially stable is meant that the cleavable linker of the antibody-drug conjugate does not undergo a significant amount of cleavage in the absence of a first enzyme and a second enzyme as described above. For example, as described above, the second cleavable moiety can protect the first cleavable moiety from being cleaved, and as such the cleavable linker of the antibody-drug conjugate does not undergo a significant amount of cleavage in the absence of a second enzyme as described above. For instance, the cleavable linker of the antibody-drug conjugate may be substantially stable such that 25% or less of the antibody-drug conjugate is cleaved in the absence of the first enzyme and/or second enzyme, such as 20% or less, or 15% or less, or 10% or less, or 5% or less, or 4% or less, or 3% or less, or 2% or less, or 1% or less. In some cases, the antibody-drug conjugate is substantially stable such that the cleavable linker of the antibody-drug conjugate does not undergo a significant amount of cleavage in the absence of the first enzyme and/or second enzyme, but can be cleaved when in the presence of the first enzyme and the second enzyme. For example, the antibody-drug conjugate can be substantially stable after administration to a subject. In some cases, the antibody-drug conjugate is substantially stable after administration to a subject, and then, when the antibody-drug conjugate is in the presence of the second enzyme at a desired site of action, the second cleavable moiety can be cleaved from the cleavable linker, thus exposing the first cleavable moiety to subsequent cleavage by the first enzyme, which in turn releases the drug at the desired site of action. In certain embodiments, after administration to a subject the antibody-drug conjugate is stable for an extended period of time in the absence of the first enzyme and/or second enzyme, such as 1 hr or more, or 2 hrs or more, or 3 hrs or more, or 4 hrs or more, or 5 hrs or more, or 6 hrs or more, or 7 hrs or more, or 8 hrs or more, or 9 hrs or more, or 10 hrs or more, or 15 hrs or more, or 20 hrs or more, or 24 hrs (1 day) or more, or 2 days or more, or 3 days or more, or 4 days or more, or 5 days or more, or 6 days or more, or 7 days (1 week) or more. In certain embodiments, the antibody-drug conjugate is stable at a range pH values for an extended period of time in the absence of the first enzyme and/or second enzyme, such as at a pH ranging from 2 to 10, or from 3 to 9, or from 4 to 8, or from 5 to 8, or from 6 to 8, or from 7 to 8.

As described above, the antibody-drug conjugates of the present disclosure find use in treatment of a condition or disease in a subject that is amenable to treatment by administration of the parent drug. By “treatment” is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g. terminated, such that the host no longer suffers from the condition, or at least the symptoms that characterize the condition. Thus treatment includes: (i) prevention, that is, reducing the risk of development of clinical symptoms, including causing the clinical symptoms not to develop, e.g., preventing disease progression to a harmful state; (ii) inhibition, that is, arresting the development or further development of clinical symptoms, e.g., mitigating or completely inhibiting an active disease; and/or (iii) relief, that is, causing the regression of clinical symptoms.

The subject to be treated can be one that is in need of therapy, where the subject to be treated is one amenable to treatment using the parent drug. Accordingly, a variety of subjects may be amenable to treatment using the antibody-drug conjugates disclosed herein. Generally, such subjects are “mammals”, with humans being of interest. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees and monkeys).

The amount of antibody-drug conjugate administered can be initially determined based on guidance of a dose and/or dosage regimen of the parent drug. In general, the antibody-drug conjugates can provide for targeted delivery and/or enhanced serum half-life of the bound drug, thus providing for at least one of reduced dose or reduced administrations in a dosage regimen. Thus, the antibody-drug conjugates can provide for reduced dose and/or reduced administration in a dosage regimen relative to the parent drug prior to being conjugated in an antibody-drug conjugate of the present disclosure.

Furthermore, as noted above, because the antibody-drug conjugates can provide for controlled stoichiometry of drug delivery, dosages of antibody-drug conjugates can be calculated based on the number of drug molecules provided on a per antibody-drug conjugate basis.

In some embodiments, multiple doses of an antibody-drug conjugate are administered. The frequency of administration of an antibody-drug conjugate can vary depending on any of a variety of factors, e.g., severity of the symptoms, condition of the subject, etc. For example, in some embodiments, an antibody-drug conjugate is administered once per month, twice per month, three times per month, every other week, once per week (qwk), twice per week, three times per week, four times per week, five times per week, six times per week, every other day, daily (qd/od), twice a day (bds/bid), or three times a day (tds/tid), etc.

### Methods of Treating Cancer

The present disclosure provides methods that include delivering a conjugate of the present disclosure to an individual having a cancer. The methods are useful for treating a wide variety of cancers, including, but not limited to breast, ovarian, colon, lung, stomach, and pancreatic cancer. In the context of cancer, the term “treating” includes one or more (e.g., each) of: reducing growth of a solid tumor, inhibiting replication of cancer cells, reducing overall tumor burden, and ameliorating one or more symptoms associated with a cancer.

Carcinomas that can be treated using a subject method include, but are not limited to, colon carcinoma, colorectal carcinoma, gastric carcinoma, lung carcinoma, including small cell carcinoma and non-small cell carcinoma of the lung, pancreatic carcinoma, breast carcinoma, ovarian carcinoma, prostate carcinoma, adenocarcinoma, cystadenocarcinoma, medullary carcinoma, renal cell carcinoma, ductal carcinoma in situ or bile duct carcinoma, choriocarcinoma, seminoma, embryonal carcinoma, cervical carcinoma, uterine carcinoma, testicular carcinoma, and epithelial carcinoma, etc.

In certain aspects, provided are methods of treating cancer in a subject, such methods including administering to the subject a therapeutically effective amount of a conjugate of the present disclosure, where the administering is effective to treat cancer in the subject. In some embodiments, the method of treating cancer includes administering to the subject a therapeutically effective amount of pharmaceutical composition including any of the conjugates of the present disclosure, where the administering is effective to treat cancer in the subject.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. By “average” is meant the arithmetic mean. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### General Synthetic Procedures

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any purification protocol known in the art, including chromatography, such as HPLC, preparative thin layer chromatography, flash column chromatography and ion exchange chromatography. Any suitable stationary phase can be used, including normal and reversed phases as well as ionic resins. In certain embodiments, the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4′ edition, Vol. 15/1, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

The subject compounds can be synthesized via a variety of different synthetic routes using commercially available starting materials and/or starting materials prepared by conventional synthetic methods. A variety of examples of synthetic routes that can be used to synthesize the compounds disclosed herein are described in the schemes below.

### Example 1

### Synthesis of Branched Linkers

### Materials and Methods

### General Information

Synthetic reagents were purchased from Sigma-Aldrich, Acros, AK Scientific, or other commercial sources and were used without purification. Anhydrous solvents were obtained from commercial sources in sealed bottles. Compounds 5, 12, 29 and 74 were obtained commercially from Shanghai Medicilon and used without purification. Cytotoxins belotecan 16 and MMAE 13 were obtained from commercial sources and used as received. In all cases, solvent was removed under reduced pressure with a Buchi Rotovapor R-114 equipped with a Buchi V-700 vacuum pump. Column chromatography was performed with a Biotage Isolera chromatography system. Preparative HPLC purifications were performed using Waters preparative HPLC unit equipped with Phenomenex Kinetex 5 m EVO C18 150×21.2 mm column. Low-resolution mass spectra (LRMS) were acquired on Agilent Technology 6120 Quadrupole LC/MS, equipped with Agilent 1260 Infinity HPLC system, G1314 variable wavelength detector, and Agilent Poroshell 120 SB C18, 4.6 mm×50 mm column at room temperature using 10-100% gradient of water and acetonitrile containing 0.1% formic acid. HPLCs were monitored at 254 or 205 nm.

### Synthesis of Branched HIPS Constructs

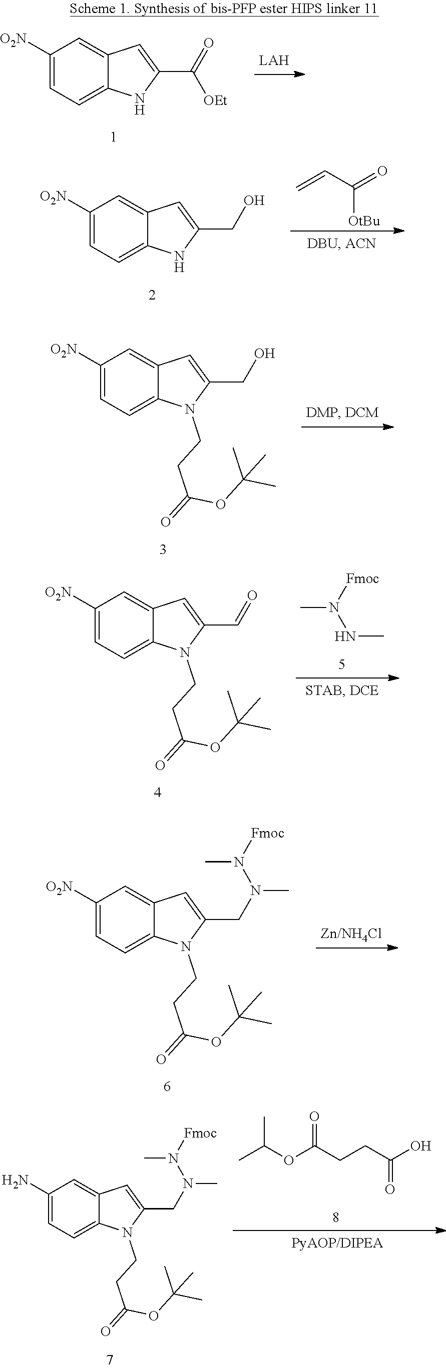

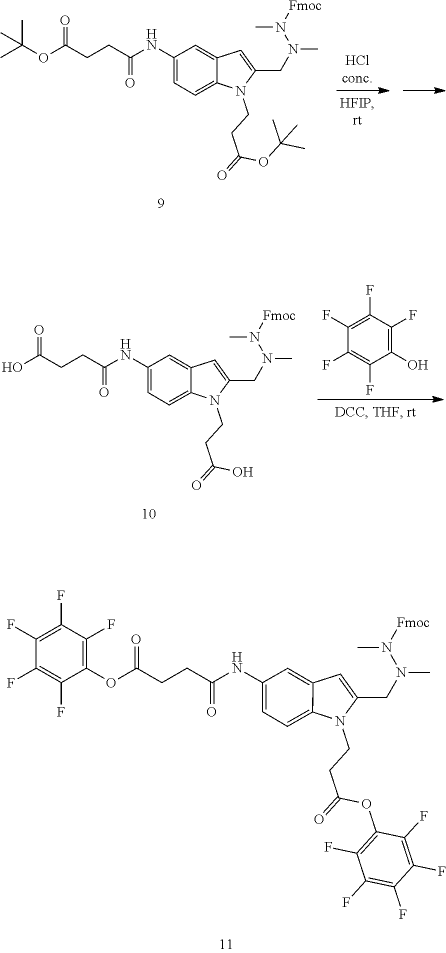

### Preparation of (5-nitro-1H-indol-2-yl)methanol (2)

To an oven-dried round-bottom flask were added ethyl 5-nitro-1H-indole-2-carboxylate (1, 234 mg, 1.0 mmol) and 5 mL of anhydrous THF. Solution was cooled down to 0° C. and treated with lithium aluminum hydride (57 mg, 1.5 mmol) in small portions over 5 minutes period with vigorous stirring. Reaction mixture was stirred for 30 minutes and quenched with 5 mL of saturated aqueous sodium bicarbonate solution, extracted with ethyl acetate (2×25 mL), washed with brine, and dried over sodium sulfate. After removal of solvent, the residue was purified on silica gel (10-50% v/v EtOAc/hexanes) to afford 115 mg (0.6 mmol, 60% yield) of product 2 as a yellow solid. LRMS (ESI): m/z 192.9 [M+H]⁺, Calcd for C₉H₈N₂O₃ m/z 193.1.

### Preparation of tert-butyl 3-(2-(hydroxymethyl)-5-nitro-1H-indol-1-yl)propanoate (3)

In an oven-dried round-bottom flask were combined 5-nitro-1H-indol-2-yl)methanol (2, 115 mg, 0.6 mmol), 5 mL of acetonitrile, 0.44 mL (3 mmol) of tert-butyl acrylate, and 90 μL of DBU (0.6 mmol). The resulting mixture was stirred at 80° C. for two hours, then diluted with 25 mL of ethyl acetate, washed sequentially with 10% aqueous citric acid solution (15 mL) and brine, dried over sodium sulfate. Solvents were removed under vacuum, and the residue was purified on silica gel (0-30% v/v EtOAc-hexane) to give 164 mg (0.51 mmol, 85% yield) of product 3 as a yellow solid. LRMS (ESI): m/z 320.9 [M+H]⁺, Calcd for C₁₆H₂₀N₂O₅ m/z 321.1.

### Preparation of tert-butyl 3-(2-formyl-5-nitro-1H-indol-1-yl)propanoate (4)

To a stirred solution of compound 3 (2.04 g, 6.37 mmol) in 35 mL of anhydrous DCM were added DMP (3.0 g, 7.0 mmol) in small portions over 5 minutes at room temperature. The resulting mixture was stirred for 30 minutes and quenched by addition of saturated sodium bicarbonate solution (20 mL). Aqueous layer was extracted with ethyl acetate (2×15 mL), combined organic layers were washed with brined and dried over sodium sulfate. After removal of solvents, the residue was purified on silica gel (0-25% v/v EtOAc-hexanes) to give 1.74 g (5.43 mmol, 85% yield) of aldehyde 4 as a yellow solid. LRMS (ESI): m/z 340.9 [M+Na]⁺, Calcd for C₁₆H₁₈N₂O₅ m/z 341.1.

### Preparation of (9H-fluoren-9-yl)methyl 2-((1-(3-(tert-butoxy)-3-oxopropyl)-5-nitro-1H-indol-2-yl)methyl)-1,2-dimethylhydrazine-1-carboxylate (6)

In an oven-dried round-bottom flask were combined aldehyde 4 (1.74 g, 5.43 mmol) and hydrazine 5 (1.54 g, 5.43 mmol) in 25 mL of anhydrous DCE at ambient temperature. The mixture was stirred for 10 minutes and treated with STAB (2.3 g, 10.8 mmol). Stirring continued for 3 hours, then reaction mixture was quenched with 20 mL of saturated sodium bicarbonate solution, extracted with EtOAc, washed with brine, and dried over sodium sulfate. After removal of solvents, the residue was purified on silica gel (0-25% v/v EtOAc-hexane) to obtain 1.71 g (2.93 mmol, 54% yield) of product 6 as a yellowish solid foam. LRMS (ESI): m/z 584.9 [M+H]⁺, Calcd for C₃₃H₃₆N₄O₆ m/z 585.3.

### Preparation of (9H-fluoren-9-yl)methyl 2-((5-amino-1-(3-(tert-butoxy)-3-oxopropyl)-1H-indol-2-yl)methyl)-1,2-dimethylhydrazine-1-carboxylate (7)

A solution of nitro compound 6 (116 mg, 0.2 mmol) in 1 mL of THF was combined with a solution of ammonium chloride (85 mg, 1.6 mmol) in 1 mL of water, and 0.5 mL of methanol at ambient temperature. The resulting mixture was treated with zinc powder (104 mg, 1.6 mmol) in several small portions. Reaction mixture was stirred vigorously for 2 hours, solids were filtered off, the residue was partitioned between saturated aqueous ammonium chloride (25 mL) and ethyl acetate (25 mL), aqueous layer was extracted with ethyl acetate (20 mL), combined organic layer was washed with brine, and dried over sodium sulfate. Removal of solvents under vacuum afforded 110 mg (0.2 mmol, quant. yield) of crude product 7 which was used further in synthesis without purification. LRMS (ESI): m/z 555.3 [M+H]⁺, Calcd for C₃₃H₃₈N₄O₄ m/z 555.3.

### Preparation of (9H-fluoren-9-yl)methyl 2-((1-(3-(tert-butoxy)-3-oxopropyl)-5-(4-(tert-butoxy)-4-oxobutanamido)-1H-indol-2-yl)methyl)-1,2-dimethylhydrazine-1-carboxylate (9)

To a mixture of crude compound 7 (110 mg, 0.2 mmol) and 4-(tert-butoxy)-4-oxobutanoic acid 8 (40 mg, 1.1 mmol) in 2 mL of DMF were added DIPEA (0.12 mL, 0.6 mmol), followed by PyAOP (110 mg, 0.2 mmol) at room temperature. After 30 minutes, reaction mixture was quenched by pouring into saturated aqueous ammonium chloride solution (15 mL), extracted with EtOAc (2×25 mL), washed with brine, dried over sodium sulfate. Solvents were removed in vacuum to give crude product 9 (120 mg, 0.17 mmol, 85% yield) as a dark oil, which was used further without purification. LRMS (ESI): m/z 733.4 [M+Na]⁺, Calcd for C₄₁H₅₀N₄O₇ m/z 733.4.

### Preparation of 4-((2-((2-(((9H-fluoren-9-yl)methoxy)carbonyl)-1,2-dimethylhydrazinyl)methyl)-1-(2-carboxyethyl)-1H-indol-5-yl)amino)-4-oxobutanoic acid (10)

Crude tert-Butyl ester 9 (300 mg, 0.42 mmol) was dissolved in 4.2 mL of hexafluoro isopropanol and treated with 0.42 mL of concentrated HCl at room temperature. Reaction mixture was stirred for 2 hours, solvent was removed under vacuum, and the residue was purified by reversed-phase flash chromatography (C18, 0-100% v/v CH3CN—H₂O with 0.05% TFA). Fractions containing product were lyophilized to give diacid 10 (143 mg, 0.24 mmol, 57% yield) as an off-white solid. LRMS (ESI): m/z 621.3 [M+Na]⁺, Calcd for C₃₃H₃₄N₄O₇ m/z 621.2.

### Preparation of (9H-fluoren-9-yl)methyl 1,2-dimethyl-2-((1-(3-oxo-3-(perfluorophenoxy)propyl)-5-(4-oxo-4-(perfluorophenoxy)butanamido)-1H-indol-2-yl)methyl)hydrazine-1-carboxylate (11)

To a mixture of diacid 10 (25 mg, 42 μmol) and pentafluorophenol (23 mg, 125 mol) in 2 mL of anhydrous THF were added DCC (17 mg, 84 μmol) at room temperature. Reaction mixture was stirred overnight, solids were filtered off, solvent was removed under reduced pressure, and the residue was purified by reversed-phase HPLC (C18, 5-95% v/v CH₃CN—H₂O with 0.05% TFA). Pure fractions were lyophilized to obtain bis-PFP ester 11 (19 mg, 20 μmol, 48% yield) as a pink solid. LRMS (ESI): m/z 931.2 [M+H]⁺, Calcd for C₄₅H₃₂F₁₀N₄O₇ m/z 931.2.

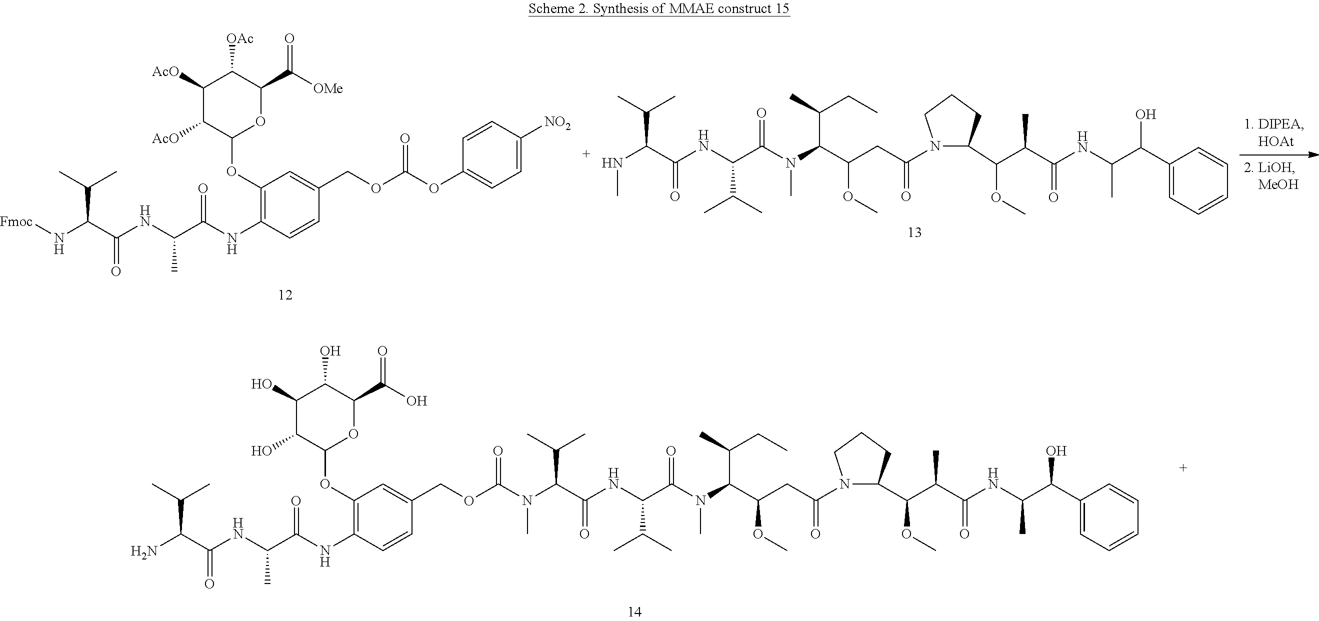

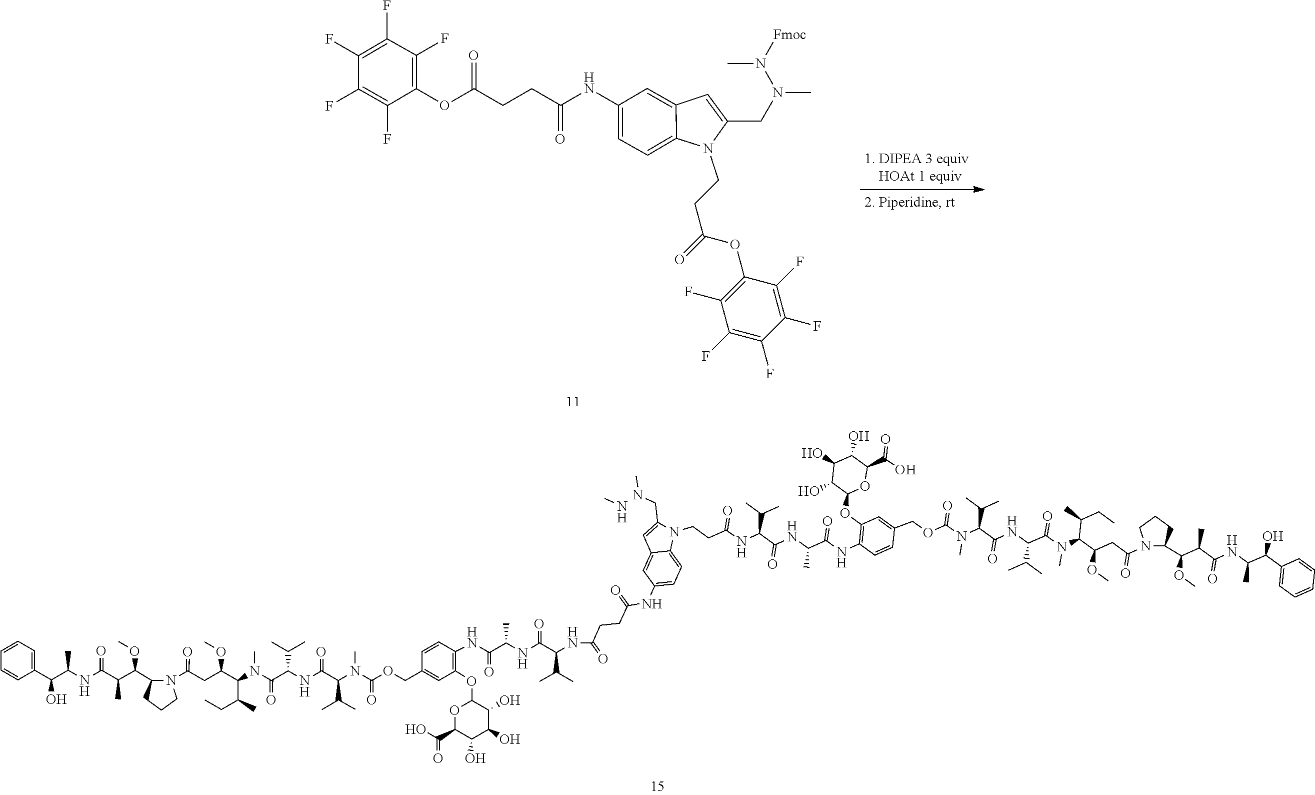

### Preparation of (2S,3S,4S,5R,6S)-6-(2-((S)-2-((S)-2-amino-3-methylbutanamido)propanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (14)

To an oven-dried 20 mL scintillation vial were added monomethyl auristatin E (MMAE, 13, 36 mg, 50 μmol) and 2 mL of anhydrous DMF, followed by 26 μL of DIPEA (150 mol) and 7 mg of HOAt (50 μmol). The resulting mixture was treated with PNP carbonate 12 (51 mg, 50 μmmol) as a solid in one portion at room temperature, stirred for 2 hours, and concentrated under vacuum to remove DMF. The residue was dissolved in 3 mL of methanol and slowly treated with 1 mL of 1M aqueous LiOH solution at 0° C. Reaction mixture was stirred for 15 minutes, then warmed up to room temperature and stirring continued for 3 hours, until hydrolysis was judged complete by LCMS analysis. Reaction mixture was quenched by addition of 1 mL of 1M HCl, concentrated under vacuum, and purified by reversed-phase HPLC (C18, 0-50% v/v CH3CN—H₂O with 0.05% TFA). Lyophilized pure fractions gave 35 mg of compound 14 (28 μmol, 57% yield) as a white powder. LRMS (ESI): m/z 1229.6 [M+H]⁺, Calcd for C₆₁H₉₆N₈O₁₈ m/z 1229.7.

### Preparation of (2S,3S,4S,5R,6S)-6-(2-((S)-2-((S)-2-(4-((1-(3-(((S)-1-(((S)-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((2S,3R,4S,5S,6S)-6-carboxy-3,4,5-trihydroxytetrahydro-2H-pyran-2-yl)oxy)phenyl)amino)-1-oxopropan-2-yl)amino)-3-methyl-1-oxobutan-2-yl)amino)-3-oxopropyl)-2-((1,2-dimethylhydrazinyl)methyl)-1H-indol-5-yl)amino)-4-oxobutanamido)-3-methylbutanamido)propanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (15)

To an oven-dried 20 mL scintillation vial were added amine 14 (34 mg, 28 μmol) and 2 mL of anhydrous DMA, followed by 15 μL of DIPEA (84 μmol) and 4 mg of HOAt (28 mol). The resulting mixture was treated with bis-PFP ester 11 (13 mg, 14 μmmol) as a solid in portion at room temperature, and let stir for 3 hours, until starting materials were fully consumed as judged by LCMS analysis. Reaction mixture was directly treated with 56 μL (0.56 mmol) of piperidine at room temperature, let stand for 20 minutes, and purified by reversed-phase HPLC (C18, 0-75% v/v CH3CN—H₂O with 0.05% TFA). Lyophilized pure fractions gave 21 mg of compound 15 (7.5 μmol, 54% yield) as a white powder. LRMS (ESI): m/z 1400.3 [M+2H]⁺⁺, Calcd for C₁₄₀H₂₁₂N₂₀O₃₉ m/z 1399.8.

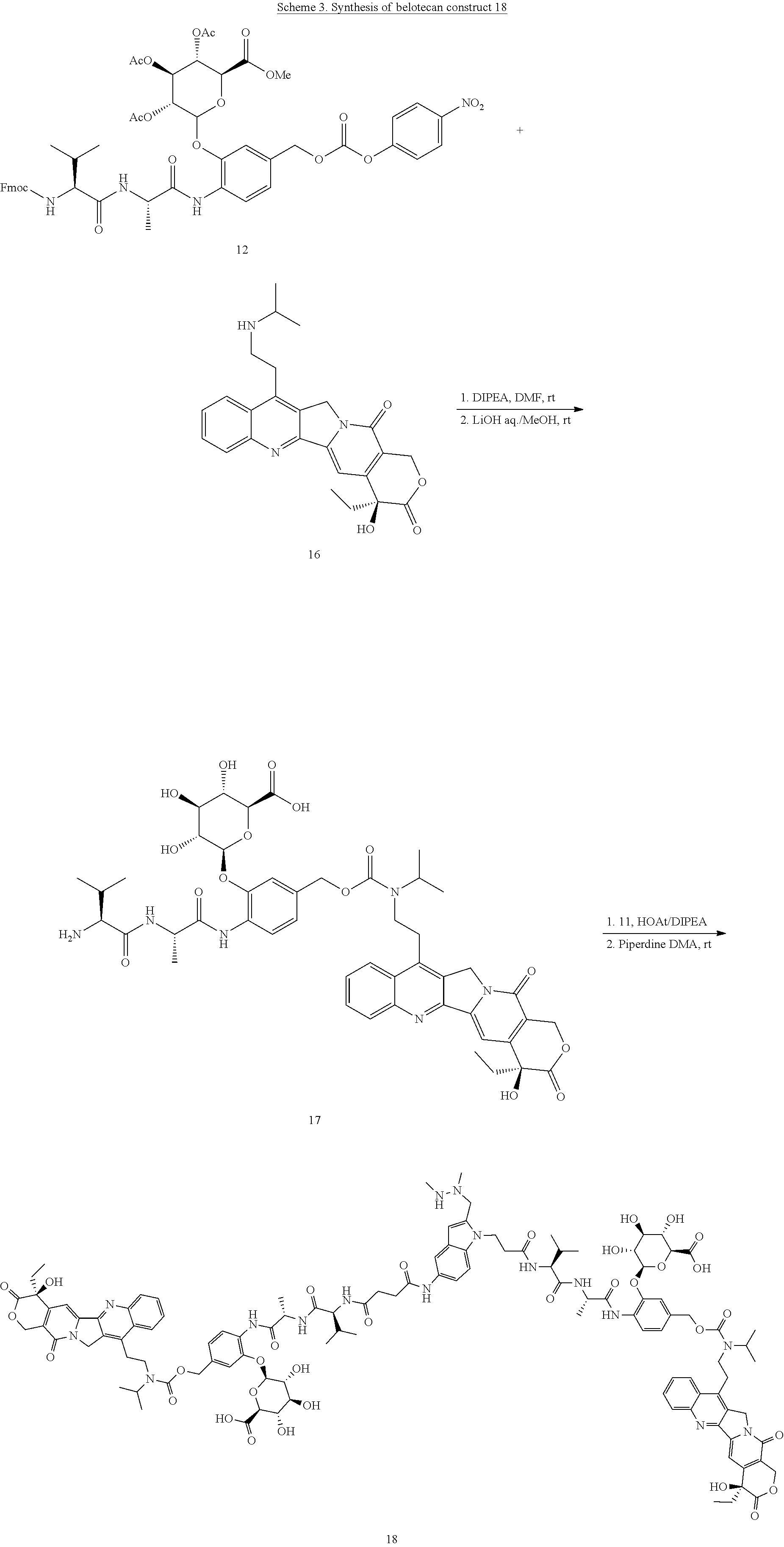

### Preparation of (2S,3S,4S,5R,6S)-6-(2-((S)-2-((S)-2-amino-3-methylbutanamido)propanamido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (17)

To a solution of belotecan 16 (HCl salt, 20 mg, 43 μmol) in 2 mL DMF were added 15 uL of DIPEA (86 μmol) and 6 mg of HOAt (43 μmol). The resulting mixture was combined with PNP carbonate 12 (43 mg, 43 μmol) at room temperature and stirred for one hour, then DMF was removed under vacuum. The residue was dissolved in 3 mL of MeOH and treated with 1 mL of 1M aqueous LiOH at ambient temperature. After 10 minutes, 1 mL of 1M aqueous HCl was added to the mixture, followed by 1 mL of 0.5M pH 4.7 acetate buffer. The resulting mixture was stirred for 30 minutes at room temperature and directly purified by reversed phase HPLC (C18 column, 0-50% v/v gradient of CH3CN/H₂O with 0.05% TFA). Solvent was removed under vacuum to give 17 mg (18 μmol, 43% yield) of compound 17 as a glassy yellow solid. LRMS (ESI): m/z 945.4 [M+H]⁺, Calcd for C₄₇H₅₆N₆O₁₅ m/z 945.4.

### Preparation of (2S,3S,4S,5R,6S)-6-(2-((S)-2-((S)-2-(4-((1-(3-(((S)-1-(((S)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1-oxopropan-2-yl)amino)-3-methyl-1-oxobutan-2-yl)amino)-3-oxopropyl)-2-((1,2-dimethylhydrazineyl)methyl)-1H-indol-5-yl)amino)-4-oxobutanamido)-3-methylbutanamido)propanamido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (18)

Compound 17 (25 mg, 26 μmol) was combined with bis-PFP ester 11 (11 mg, 12 μmol), 4 mg of HOAt (12 μmol), and 14 μL of DIPEA (78 μmol) in 2 mL of DMA at room temperature. After one hour, piperidine 24 μL, 0.24 mmol) was added to the reaction mixture. After 30 minutes, reaction mixture was directly purified by reversed phase HPLC (C18 column, 0-50% v/v gradient of CH3CN/H₂O with 0.05% TFA). Lyophilization of pure fractions gave 15 mg of compound 18 (7 μmol, 58% yield) as a yellowish powder. LRMS (ESI): m/z 1116.1 [M+2H]⁺⁺, Calcd for C₁₁₂H₁₃₂N₁₆O₃₃ m/z 1116.0.

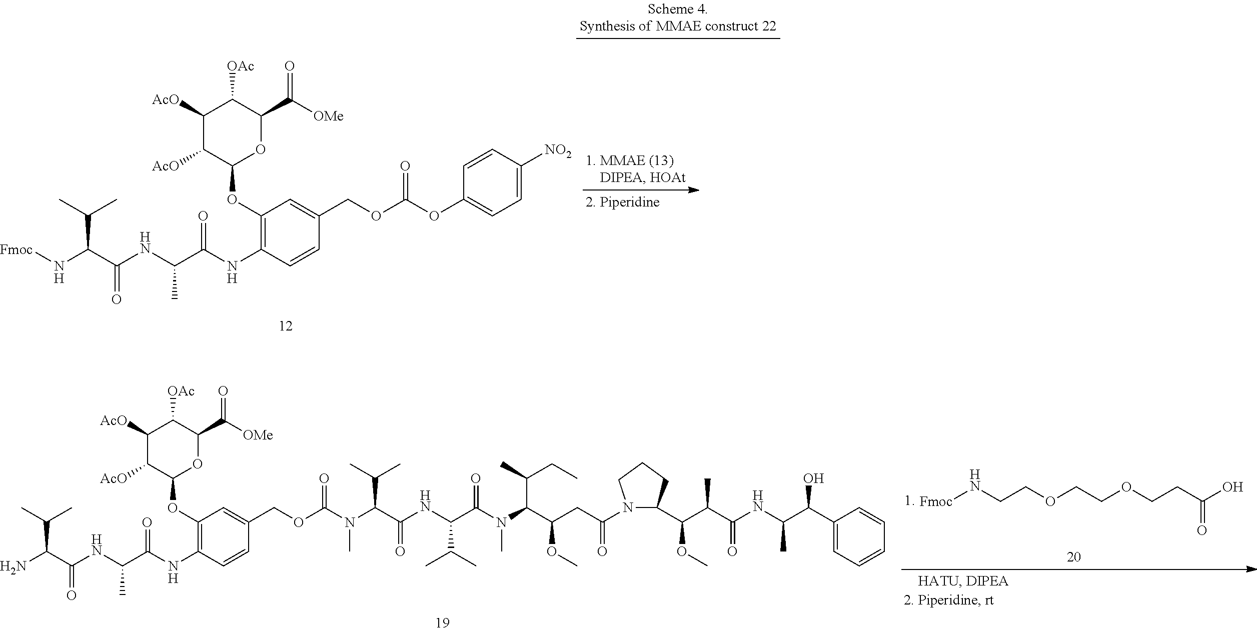

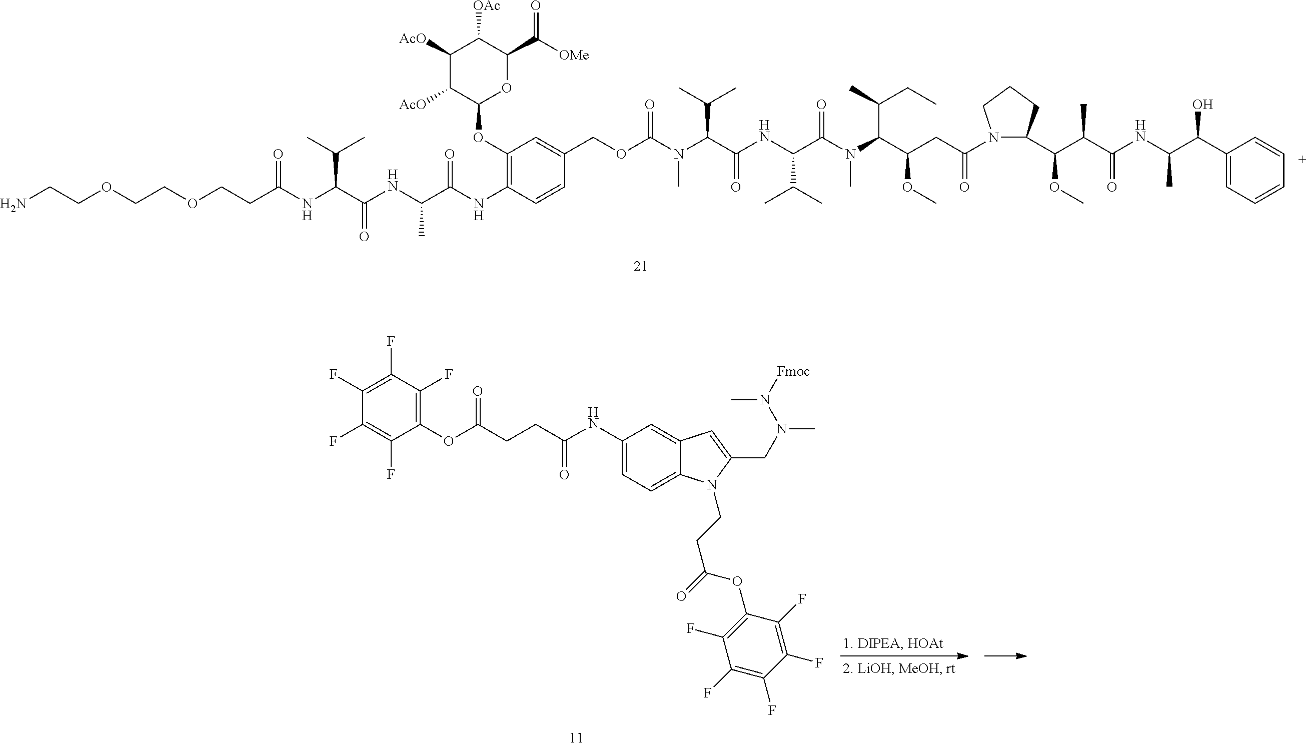

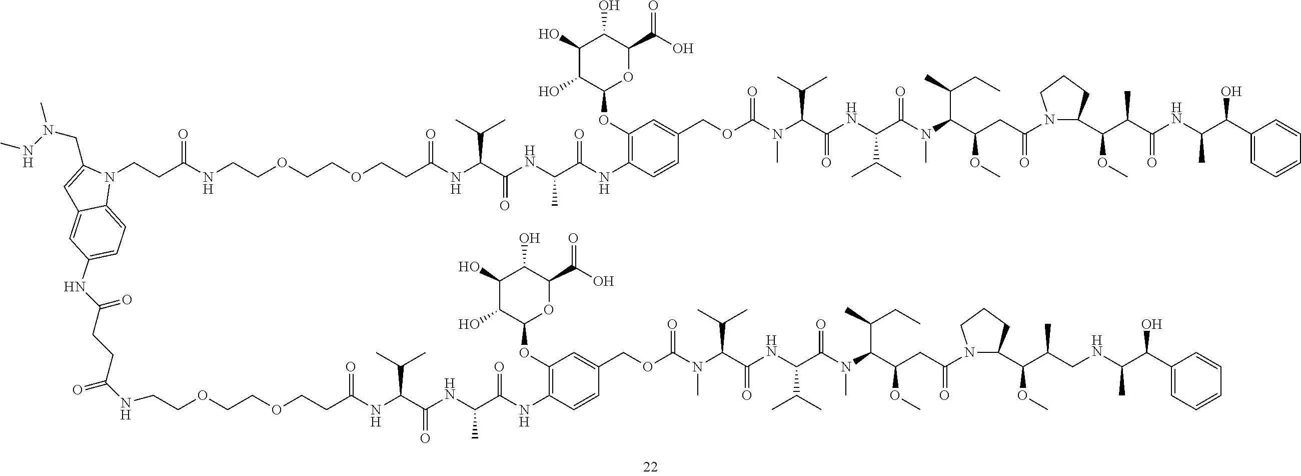

### Preparation of (2S,3R,4S,5S,6S)-2-(2-((2S,5S)-]5-amino-5-isopropyl-2-methyl-4,7-dioxo-10,13-dioxa-3,6-diazapentadecanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (21)

To a mixture of amine 19 (30 mg, 22 μmol), carboxylic acid 20 (10 mg, 25 μmol), and DIPEA (9 μL, 52 μmol) in 1 mL of DMF were added HATU (10 mg, 25 μmol) at room temperature. After one hour, reaction mixture was treated directly with piperidine (40 μL, 0.4 mmol), stirred for 20 minutes, and purified by reversed-phase HPLC (C18 column, 10-70% v/v gradient of CH₃CN/H₂O with 0.05% TFA). Fractions containing the desired product were lyophilized to give 10 mg (7 μmol, 32% yield) of compound 21 as a white powder. LRMS (ESI): m/z 1529.8 [M+H]⁺, Calcd for C₇₅H₁₁₇N₉O₂₄ m/z 1528.8.

### Preparation of (2S,3S,4S,5R,6S)-6-(2-((2S,5S)-19-(5-((2S,5S)-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((2S,3R,4S,5S,6S)-6-carboxy-3,4,5-trihydroxytetrahydro-2H-pyran-2-yl)oxy)phenyl)amino)-5-isopropyl-2-methyl-1,4,7,17-tetraoxo-10,13-dioxa-3,6,16-triazaicosan-20-amido)-2-((1,2-dimethylhydrazinyl)methyl)-1H-indol-1-yl)-5-isopropyl-2-methyl-4,7,17-trioxo-10,13-dioxa-3,6,16-triazanonadecanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (22)

To a mixture of compound 21 (3.6 mg, 2.4 μmol), DIPEA (2 μL, 12 μmol), and HOAt (0.3 mg, 2.4 μmol) in 1 mL of DMF were added bis-PFP ester 11 (1.1 mg, 1.2 μmol) at room temperature. After one hour, DMF was removed under vacuum, the residue was dissolved in 0.5 mL of methanol, and slowly treated with 0.75 mL of 1M aqueous LiOH solution at 0° C. Reaction mixture was stirred for 1 hour and purified by reversed-phase HPLC (C18 column, 10-65% v/v gradient of CH₃CN/H₂O with 0.05% TFA) to obtain 1.0 mg (0.3 μmol, 25% yield) of compound 22 as a white powder after lyophilization. LRMS (ESI): m/z 1559.4 [M+2H]⁺⁺, Calcd for C₁₅₄H₂₃₈N₂₂O₄₅ m/z 1558.9.

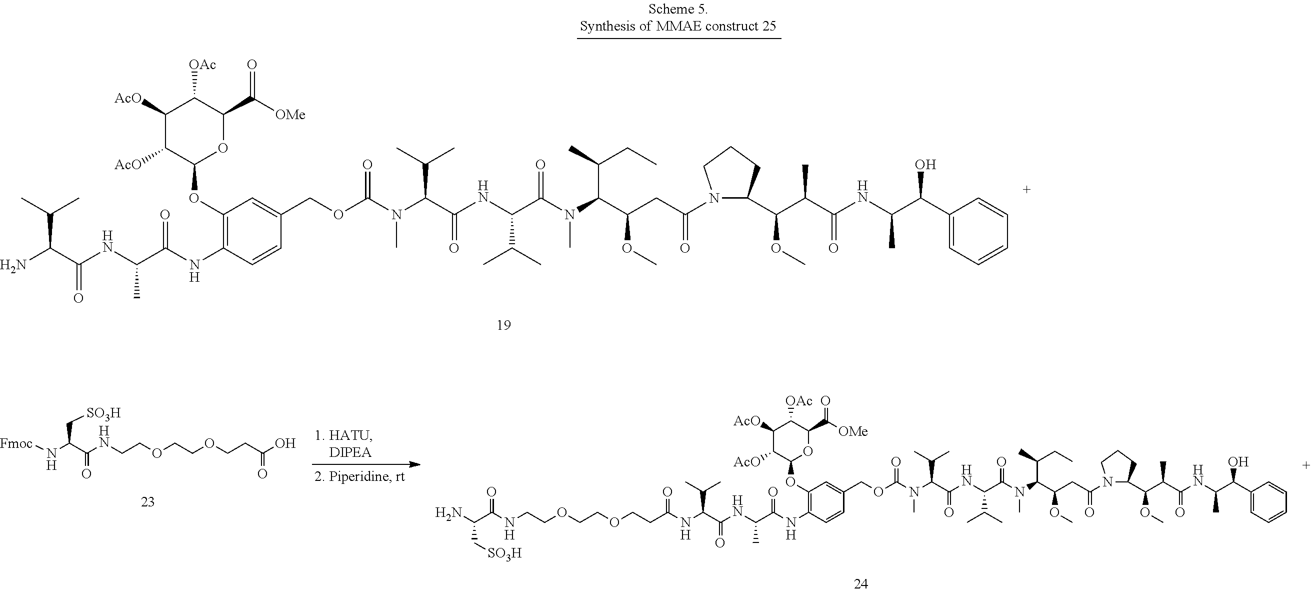

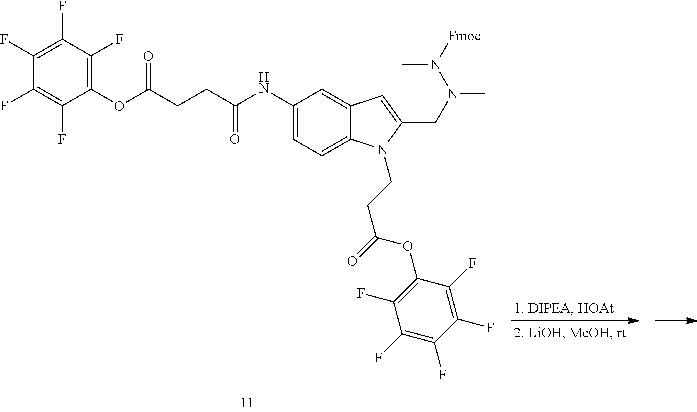

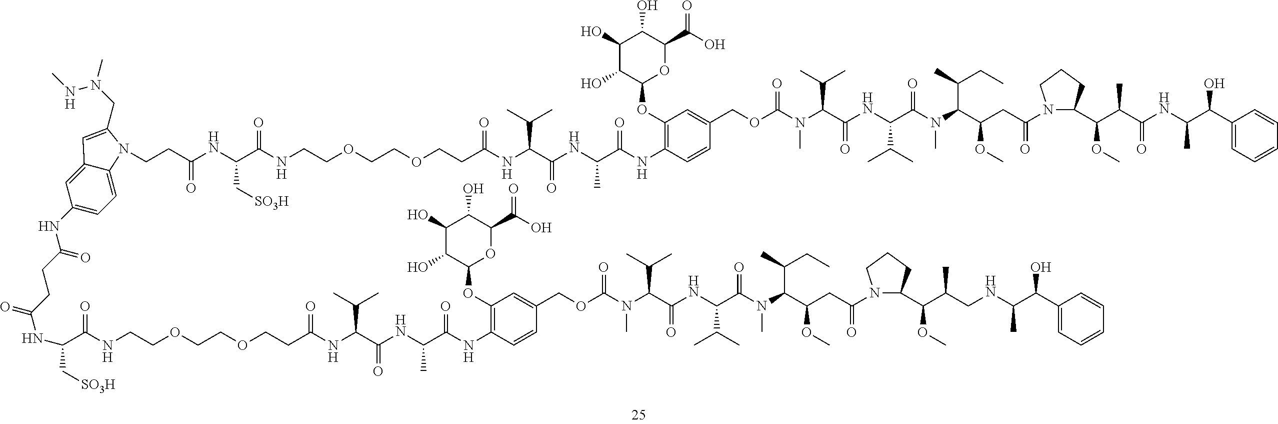

### Synthesis of (R)-1-(9H-fluoren-9-yl)-3,6-dioxo-5-(sulfomethyl)-2,10,13-trioxa-4,7-diazahexadecan-16-oic acid (23)

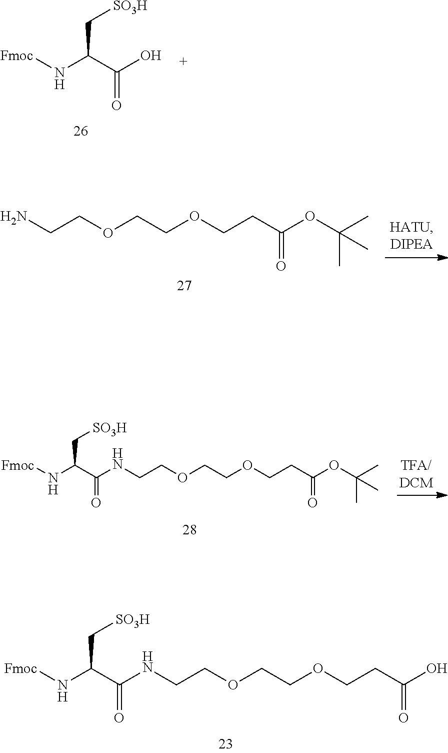

To a stirred mixture of Fmoc-L-cysteic acid 26 (200 mg, 0.51 mmol), amino-PEG2-COOtBu 27 (120 mg, 0.52 mmol), and DIPEA (0.27 mL, 1.5 mmol) in 3 mL of anhydrous DMF were added HATU (197 mg, 0.51 mmol) in one portion at room temperature. The resulting mixture was stirred for 2 hours and purified by reversed-phase chromatography (C18 column, 0-100% v/v gradient of CH₃CN/H₂O with 0.05% TFA) to give 280 mg (0.46 mmol, 91% yield) of tert-butyl ester 28. LRMS (ESI): m/z 629.2 [M+Na]⁺, Calcd for C₂₉H₁₃₈N₂O₁₀S m/z 629.2.

Compound 28 (280 mg, 0.46 mmol) was dissolved in 1 mL of DCM and treated with 0.3 mL of TFA at room temperature. After 30 minutes, solvents were removed under vacuum to give carboxylic acid 23 (230 mg, 0.42 mmol, 90% yield) as a colorless solid which was used further without purification. LRMS (ESI): m/z 551.2 [M+H]⁺, Calcd for C₂₅H₃₀N₂O₁₀S m/z 551.2.

### Preparation of (2S,5S,18R)-18-amino-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((2S,3R,4S,5S,6S)-3,4,5-triacetoxy-6-(methoxycarbonyl)tetrahydro-2H-pyran-2-yl)oxy)phenyl)amino)-5-isopropyl-2-methyl-1,4,7,17-tetraoxo-10,13-dioxa-3,6,16-triazanonadecane-19-sulfonic acid (24)

To a mixture of amine 19 (22 mg, 16 μmol), carboxylic acid 23 (11 mg, 19 μmol), and DIPEA (7 μL, 40 μmol) in 1 mL of DMF were added HATU (7 mg, 19 μmol) at room temperature. Reaction mixture was stirred for 1 hour, then directly treated with piperidine (40 μL, 0.4 mmol). After 30 minutes, reaction n mixture was purified by reversed-phase HPLC (C₁₈ column, 10-70% v/v gradient of CH₃CN/H₂O with 0.05% TFA) to give 18 mg (11 μmol, 68% yield) of compound 24 as a colorless solid. LRMS (ESI): m/z 1680.7 [M+H]⁺, Calcd for C₇₈H₁₂₂N₁₀O₂₈S m/z 1679.8.

### Preparation of MMAE construct 25

To a mixture of compound 24 (4 mg, 2.4 μmol), DIPEA (1.6 μL, 10 μmol), and HOAt (0.3 mg, 2.4 μmol) in 1 mL DMF were added bis-PFP ester 11 (1.1 mg, 1.2 μmol) in four portions over 5 minutes. The resulting mixture was stirred for 1 hour and concentrated under vacuum. The residue was dissolved in 0.5 mL of methanol, cooled down to 0° C., and treated with 0.75 mL of 1M aqueous LiOH solution. Reaction mixture was stirred at 0° C. for one hour, then purified by reversed-phase HPLC (C18, 10-70% v/v gradient of CH₃CN/H₂O with 10 mM ammonium formate). Pure fractions were combined and lyophilized to give 1.0 mg of compound 25 as a white powder (0.3 μmol, 25% yield). LRMS (ESI): m/z 1710.5 [M+2H]⁺⁺, Calcd for C₁₆₀H₂₄₈N₂₄O₅₃S₂ m/z 1709.8.

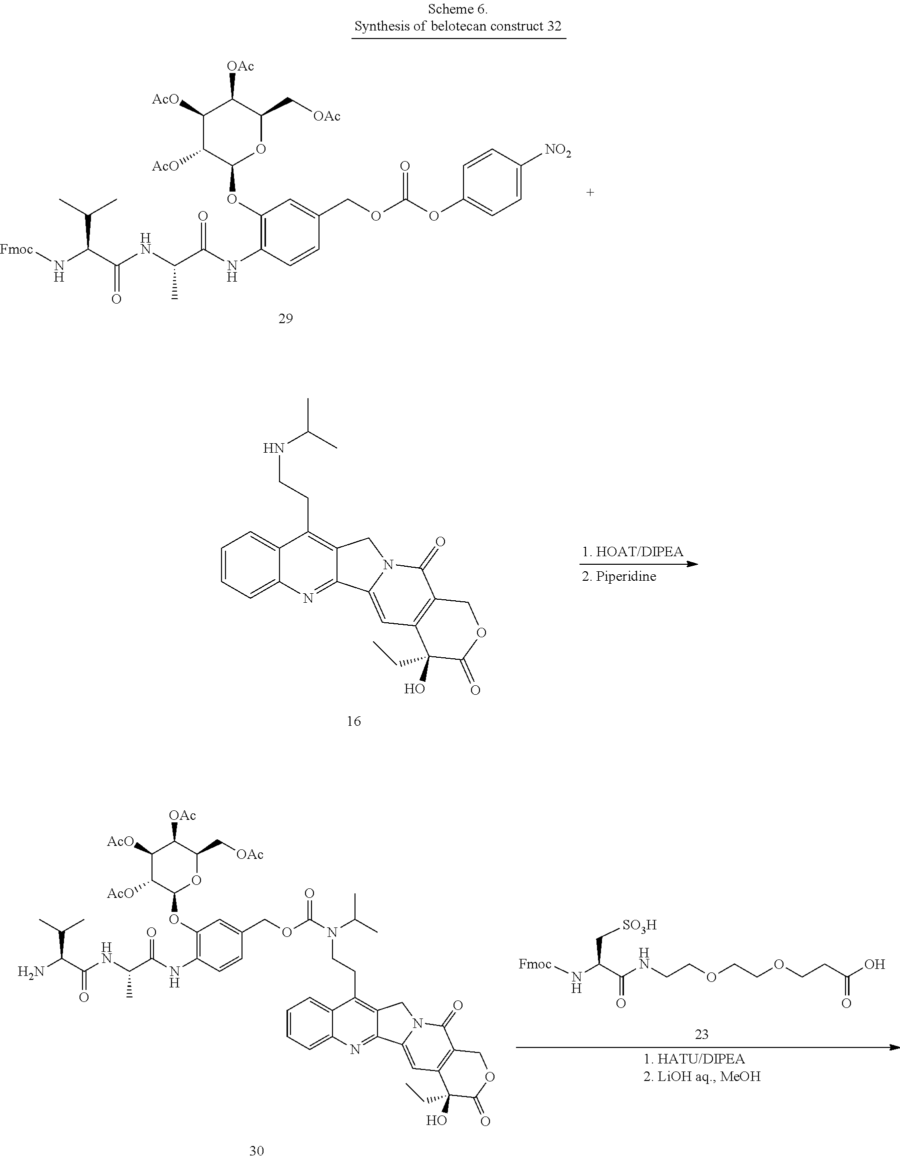

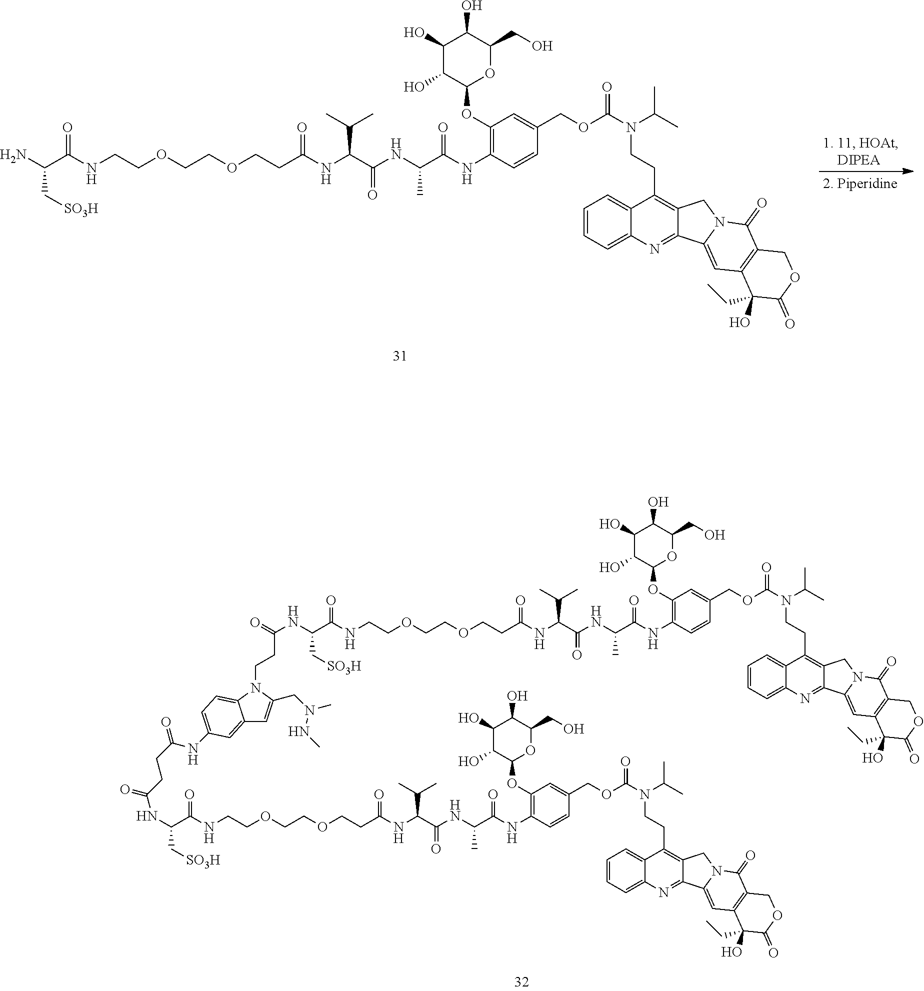

### Preparation of (2R,3S,4S,5R,6S)-2-(acetoxymethyl)-6-(2-((S)-2-((S)-2-amino-3-methylbutanamido)propanamido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)tetrahydro-2H-pyran-3,4,5-triyl triacetate (30)

To an oven-dried 20 mL scintillation vial were added Belotecan HCl (16, 48 mg, 102 mol) and 1.6 mL of anhydrous DMF, followed by 47 μL of DIPEA (269 μmol) and 13 mg of HOAt (96 μmol). The resulting mixture was treated with PNP carbonate 29 (104 mg, 101 mmol) as a solid in one portion at room temperature, stirred overnight. After starting material was consumed, 200 μL piperidine (2 mmol) was added. The mixture was stirred for 30 minutes and was monitored by LC-MS. The reaction mixture was purified by reversed-phase Biotage® (C18, 0-100% v/v CH3CN—H₂O with 0.05% TFA). Lyophilized pure fractions gave 100 mg of compound 30 (91 μmol, 90% yield) as a yellow powder. LRMS (ESI): m/z 1099.4 [M+H]⁺, Calcd for C₅₅H₆₇N₆O₁₈ m/z 1099.5.

### Preparation of (2S,5S,18R)-18-amino-1-((4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)-2-(((2S,3R,4S,5R,6R)-3,4,5-trihydroxy-6-(hydroxymethyl)tetrahydro-2H-pyran-2-yl)oxy)phenyl)amino)-5-isopropyl-2-methyl-1,4,7,17-tetraoxo-10,13-dioxa-3,6,16-triazanonadecane-19-sulfonic acid (31)

To an oven-dried 20 mL scintillation vial were added amine (30, 50 mg, 46 μmol) cysteic acid linker (23, 27 mg, 49 μmol) and 0.5 mL of anhydrous DMF, followed by 24 μL of DIPEA (138 μmol) and 18 mg of HATU (46 μmol). The resulting mixture was stirred at room temperature and was monitored by LC-MS. After starting material was consumed, the solution was concentrated under vacuum to remove DMF. The residue was dissolved in 1 mL of methanol and slowly treated with 1.5 mL of 1M aqueous LiOH solution at 0° C. Reaction mixture was stirred for 15 minutes, then warmed up to room temperature and stirring continued for 1 hours, until hydrolysis was judged complete by LCMS analysis. Reaction mixture was quenched by addition of 1 mL of 1M HCl, followed by 1 mL of 0.5M pH 4.7 acetate buffer, concentrated under vacuum, and purified by reversed-phase HPLC (C18, 0-75% v/v CH₃CN—H₂O with 0.05% TFA). Lyophilized pure fractions gave 39 mg of compound 31 (31 μmol, 68% yield) as a yellow powder. LRMS (ESI): m/z 1241.5 [M+H]⁺, Calcd for C₅₇H₇₇N₈O₂₁S m/z 1241.5.

### Preparation of (2S,5S,18R)-18-(4-((2-((1,2-dimethylhydrazineyl)methyl)-1-((2S,5S,18R)-1-((4-((((2-((S)-4-ethyl-4-hydroxy-3,14-dioxo-3,4,12,14-tetrahydro-1H-pyrano[3′,4′:6,7]indolizino[1,2-b]quinolin-1-yl)ethyl)(isopropyl)carbamoyl)oxy)methyl)-2-(((2S,3R,4S,5R,6R)-3,4,5-trihydroxy-6-(hydroxymethyl)tetrahydro-2H-pyran-2-yl)oxy)phenyl)amino)-5-isopropyl-2-methyl-1,4,7,17,20-pentaoxo-18-(sulfomethyl)-10,13-dioxa-3,6,16,19-tetraazadocosan-22-yl)-1H-indol-5-yl)amino)-4-oxobutanamido)-1-((4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)-2-(((2S,3R,4S,5R,6R)-3,4,5-trihydroxy-6-(hydroxymethyl)tetrahydro-2H-pyran-2-yl)oxy)phenyl)amino)-5-isopropyl-2-methyl-1,4,7,17-tetraoxo-10,13-dioxa-3,6,16-triazanonadecane-19-sulfonic acid (32)

Compound 31 (39 mg, 31 μmol) was combined with bis-PFP ester 11 (14.7 mg, 15.5 μmol), 5 mg of HOAt (31 μmol), and 17 μL of DIPEA (93 μmol) in 1 mL of DMF at room temperature. After one hour, piperidine 61 μL (0.62 mmol) was added to the reaction mixture. After 30 minutes, reaction mixture was directly purified by reversed phase HPLC (C18 column, 0-75% v/v gradient of CH₃CN/H₂O with 0.05% TFA). Lyophilization of pure fractions gave 29 mg of compound 32 (10.3 μmol, 66% yield) as a yellowish powder. LRMS (ESI): m/z 1412.1 [M+2H]⁺⁺, Calcd for C₁₃₂H₁₇₄N₂₀O₄₅S₂ m/z 1412.1.

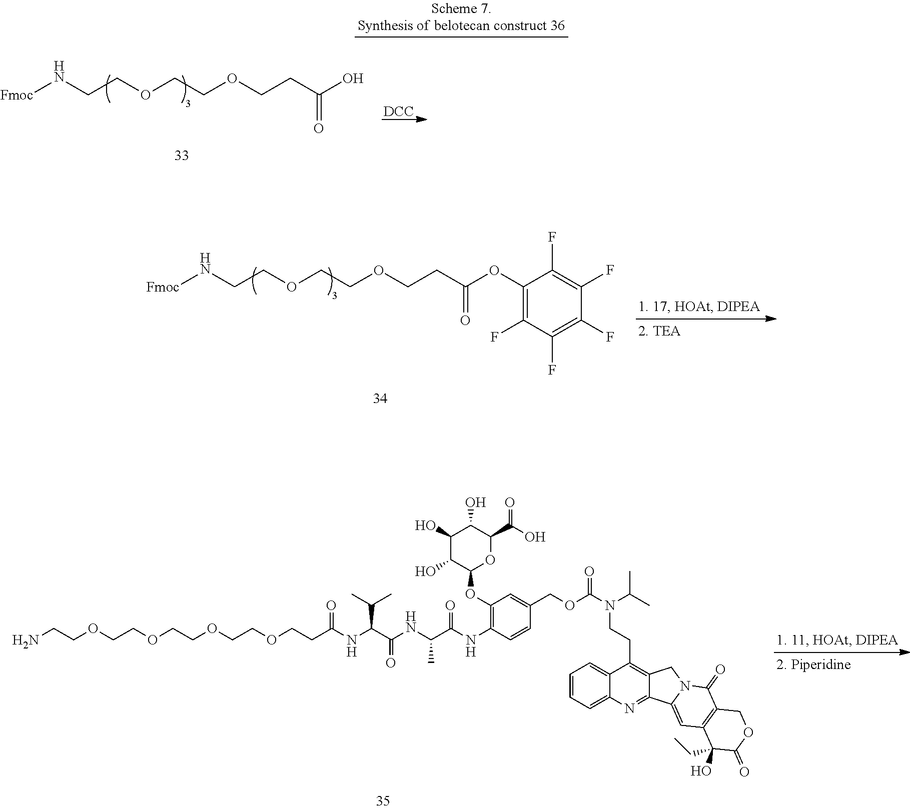

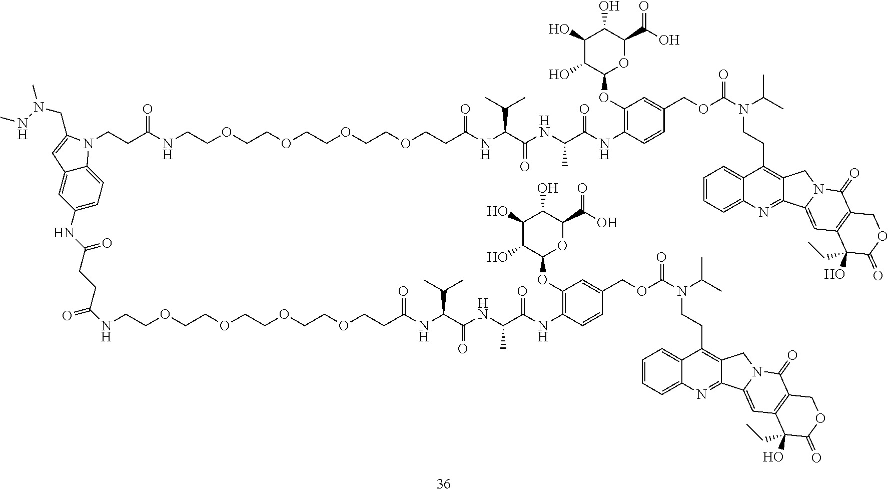 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCOCCOCCOCCOCCNC(=O)CCC(=O)Nc2ccc3c(c2)cc(CN(C)NC)n3CCC(=O)NCCOCCOCCOCCOCCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)c(O[C@@H]2O[C@H](C(=O)O)[C@@H](O)[C@H](O)[C@H]2O)c1)C(C)C]

### Preparation of perfluorophenyl 1-(9H-fluoren-9-yl)-3-oxo-2,7,10,13,16-pentaoxa-4-azanonadecan-19-oate (34)

In an oven-dried scintillation vial were combined 1-(9H-fluoren-9-yl)-3-oxo-2,7,10,13,16-pentaoxa-4-azanonadecan-19-oic acid (33, 487 mg, 1 mmol) and pentafluorophenol (368 mg, 2 mmol) in 5 mL of anhydrous THF. The resulting mixture was treated with DCC (247 mg, 1.2 mmol) in one portion at room temperature, and reaction mixture was stirred overnight. Precipitated solids were filtered off, solvents removed under vacuum, and the residue was purified by reversed-phase chromatography (C18 column, 10-100% v/v gradient of CH₃CN/H₂O with 0.05% TFA) to give 670 mg of PFP ester 34 (570 mg, 0.87 mmol, 87% yield) as a colorless oil. LRMS (ESI): m/z 654.2 [M+H]⁺, Calcd for C₃₂H₃₂F₅NO₈ m/z 654.2.

### Preparation of (2S,3S,4S,5R,6S)-6-(2-((17S,20S)-1-amino-17-isopropyl-20-methyl-15,18-dioxo-3,6,9,12-tetraoxa-16,19-diazahenicosan-21-amido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (35)

Compound 17 (262 mg, 0.22 mmol) was dissolved in 4 mL of DMF. To this solution were added DIPEA (105 μL, 0.66 mmol) and PFP ester 34 (181 mg, 0.22 mmol) as a solution in 0.5 mL of DMF, followed by HOAt (38 mg, 0.22 mmol). The resulting mixture was allowed to stand at room temperature for one hour, then treated directly with 4 mL of triethylamine. Reaction mixture was stirred for 5 hours, until Fmoc-deprotection was complete as judged by LCMS analysis. Reaction mixture was concentrated under vacuum and purified by reversed-phase chromatography (C18 column, 0-50% v/v gradient of CH₃CN/H₂O with 0.05% TFA) 185 mg (0.16 mmol, 73% yield) of compound 35 as a yellow powder. LRMS (ESI): m/z 1192.5 [M+H]⁺, Calcd for C₅₈H₇₇N₇O₂ m/z 1192.5.

### Preparation of (2S,3S,4S,5R,6S)-6-(2-((2S,5S)-25-(5-((2S,5S)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-5-isopropyl-2-methyl-1,4,7,23-tetraoxo-10,13,16,19-tetraoxa-3,6,22-triazahexacosan-26-amido)-2-((1,2-dimethylhydrazinyl)methyl)-1H-indol-1-yl)-5-isopropyl-2-methyl-4,7,23-trioxo-10,13,16,19-tetraoxa-3,6,22-triazapentacosanamido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (36)

Compound 35 (23 mg, 19 μmol) was dissolved in 2 mL of anhydrous DMA. To this solution were added DIPEA (10 μL, 57 μmol) and PFP ester 11 (8 mg, 8.6 μmol) as a solid in one portion at room temperature, followed by HOAt (2.6 mg, 19 μmol). The resulting mixture was allowed to stand at room temperature for one hour, then treated directly with 17 μL of piperidine (172 μmol). After 20 minutes, reaction mixture was purified by reversed-phase chromatography HPLC (C18 column, 0-50% v/v gradient of CH₃CN/H₂O with 0.05% TFA). Pure fractions were lyophilized to give 5.8 mg (2.1 μmol, 24% yield) of compound 36 as a yellow powder. LRMS (ESI): m/z 1363.1 [M+2H]⁺⁺, Calcd for C₁₃₄H₁₇₄N₁₈O₄₃ m/z 1362.6.

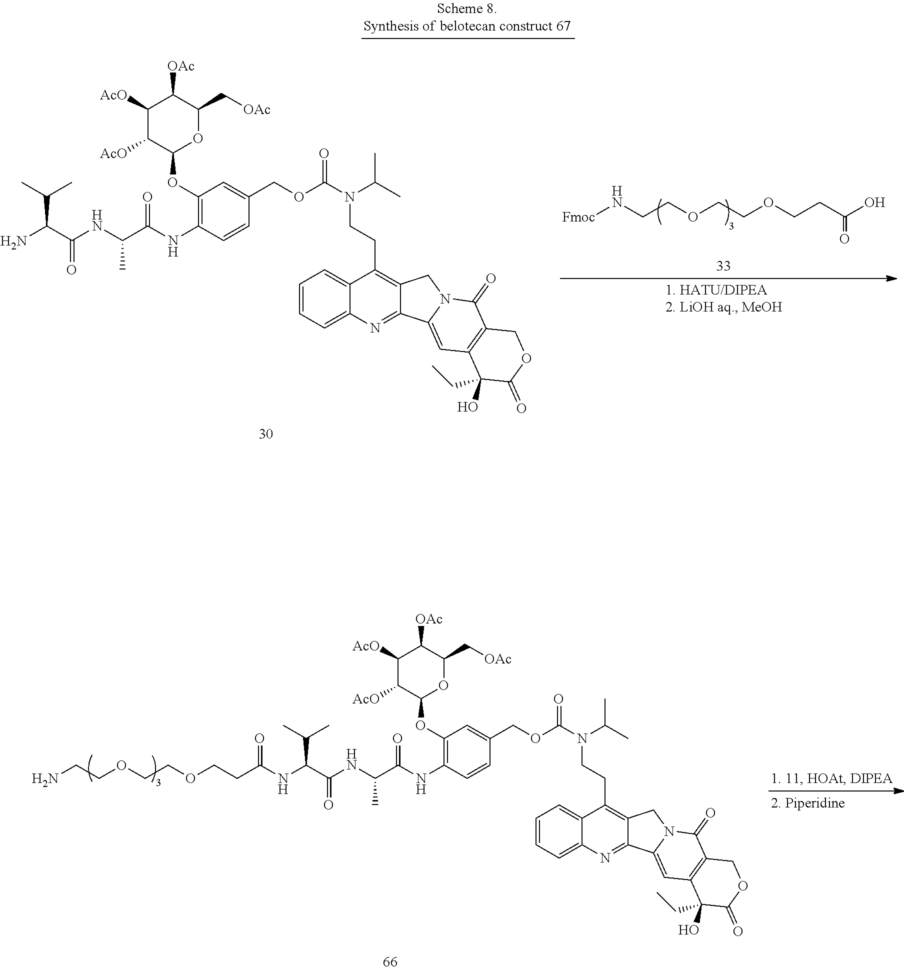

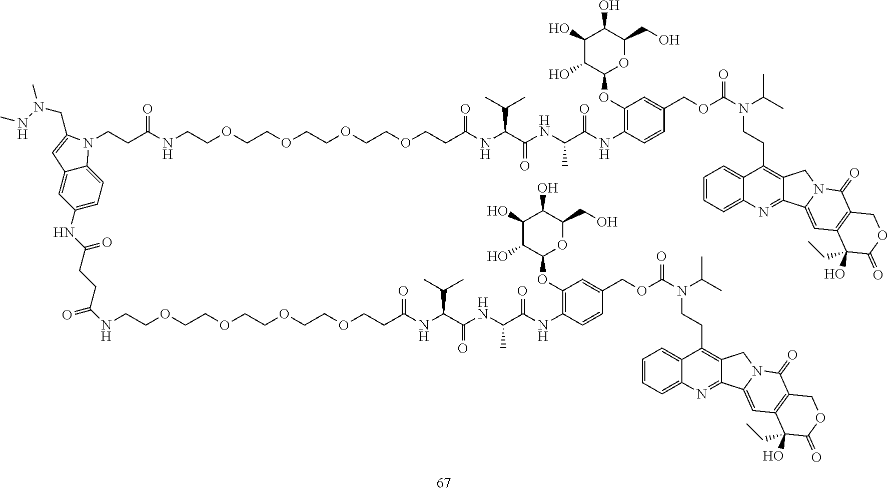 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCOCCOCCOCCOCCNC(=O)CCC(=O)Nc2ccc3c(c2)cc(CN(C)NC)n3CCC(=O)NCCOCCOCCOCCOCCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](CO)[C@H](O)[C@H](O)[C@H]2O)C(C)C)C(C)C)c(O[C@@H]2O[C@H](CO)[C@H](O)[C@H](O)[C@H]2O)c1)C(C)C]

### Preparation of 4-((17S,20S)-1-amino-17-isopropyl-20-methyl-15,18-dioxo-3,6,9,12-tetraoxa-16,19-diazahenicosan-21-amido)-3-(((2S,3R,4S,5R,6R)-3,4,5-trihydroxy-6-(hydroxymethyl)tetrahydro-2H-pyran-2-yl)oxy)benzyl (2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamate (66)

To a mixture of compounds 30 (30 mg, 27 μmol) and 33 (17 mg, 35 μmol) in DMF (0.5 mL) were added HATU (12 mg, 32 μmol), followed by DIPEA (14 μL, 82 μmol) at room temperature, and the resulting solution was stirred for 1 h. Solvent was removed under reduced pressure, and the residue was dissolved in MeOH (1 mL). To this solution was then added 1 M aqueous LiOH solution (1 mL) at 0° C., and the reaction mixture was allowed to slowly warm up to room temperature. After hydrolysis was judged complete by LCMS analysis, reaction mixture was quenched with pH 4.7 acetate buffer (1 mL). Solids were filtered off, filtrate was purified by reversed-phase prep HPLC (C18 column, 0-75% acetonitrile-water with 0.05% TFA). Pure fractions were collected and lyophilized to give product 66 as a yellow solid (19 mg, 16 μmol, 59% yield). LRMS (ESI): m/z 1178.5 [M+H]⁺, Calcd for C₅₈H₇₉N₇O₁₉ m/z: 1178.5.

### Preparation of 4-((2S,5S)-25-(2-((1,2-dimethylhydrazinyl)methyl)-5-((2S,5S)-1-((4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)-2-(((2S,3R,4S,5R,6R)-3,4,5-trihydroxy-6-(hydroxymethyl)tetrahydro-2H-pyran-2-yl)oxy)phenyl)amino)-5-isopropyl-2-methyl-1,4,7,23-tetraoxo-10,13,16,19-tetraoxa-3,6,22-triazahexacosan-26-amido)-1H-indol-1-yl)-5-isopropyl-2-methyl-4,7,23-trioxo-10,13,16,19-tetraoxa-3,6,22-triazapentacosanamido)-3-(((2S,3R,4S,5R,6R)-3,4,5-trihydroxy-6-(hydroxymethyl)tetrahydro-2H-pyran-2-yl)oxy)benzyl (2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamate (67)

To a solution of compound 66 (19 mg, 16 μmol) in DMF (0.5 mL) were added DIPEA (9 μL, 48 μmol and HOAt (3 mg, 21 μmol), followed by bis-PFP ester 11 (7.4 mg, 8 mol) at room temperature. The resulting mixture was stirred for 1 h, until coupling was judged complete by LCMS analysis. Piperidine (32 μL, 0.32 mmol) was then added directly to the reaction mixture at rt and stirring continued for 15 minutes. Reaction mixture was then purified by reversed-phase prep HPLC (C18 column, 0-70% acetonitrile-water with 0.05% TFA). Pure fractions were collected and lyophilized to obtain product 67 as a yellow solid (13 mg, 4.8 μmol, 60% yield). LRMS (ESI): m/z 1349.0 [M+2H]²⁺, Calcd for C₁₃4H₁₇₈N₁₈O₄₁ m/z: 1349.1.

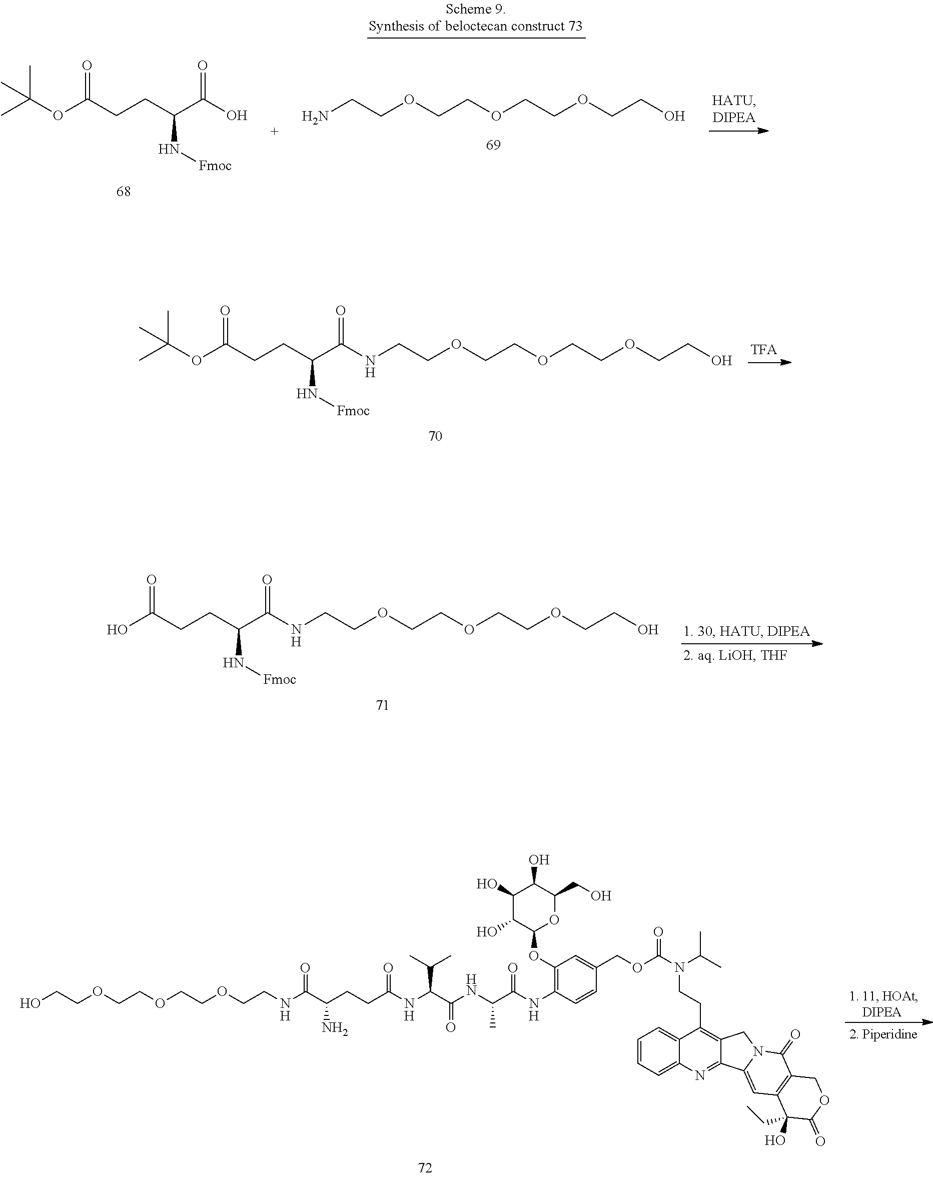

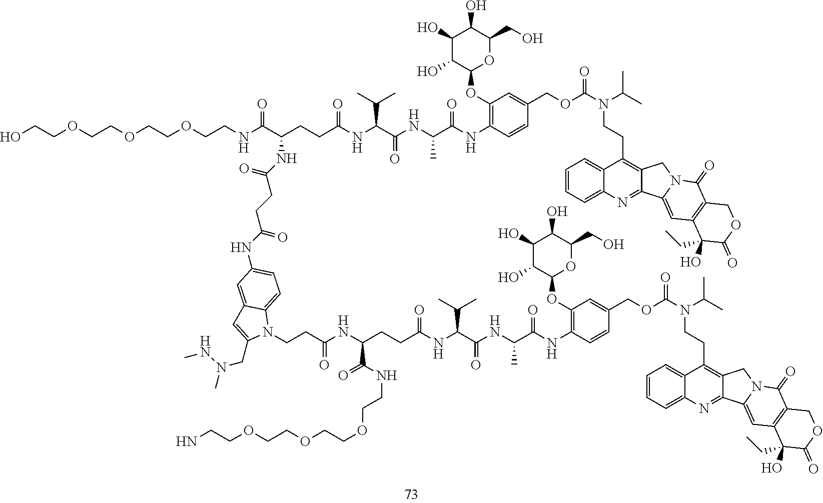 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CC[C@H](NC(=O)CCC(=O)Nc2ccc3c(c2)cc(CN(C)NC)n3CCC(=O)N[C@@H](CCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](CO)[C@H](O)[C@H](O)[C@H]2O)C(C)C)C(=O)NCCOCCOCCOCCN)C(=O)NCCOCCOCCOCCO)C(C)C)c(O[C@@H]2O[C@H](CO)[C@H](O)[C@H](O)[C@H]2O)c1)C(C)C]

### Preparation of tert-butyl (S)-14-((((9H-fluoren-9-yl)methoxy)carbonyl)amino)-1-hydroxy-13-oxo-3,6,9-trioxa-12-azaheptadecan-17-oate (70)

To a mixture of Fmoc-Glu(OtBu)-OH (68, 42 mg, 0.1 mmol) and amino-PEG4-OH (69, 19 mg, 0.1 mmol) in DMF (1 mL) were added HATU (38 mg, 0.1 mmol) and DIPEA (52 L, 0.3 mmol) at room temperature. Reaction mixture was stirred for 1 h and directly purified by reversed-phase chromatography (C18 column, 0-70% acetonitrile-water with 0.05% TFA). Pure fractions were lyophilized to give compound 70 as a white solid (50 mg, 0.83 mmol, 83% yield). LRMS (ESI): m/z 601.3 [M+H]⁺, Calcd for C₃₂H₄₄N₂O₉ m/z: 601.3.

### Preparation of (S)-14-((((9H-fluoren-9-yl)methoxy)carbonyl)amino)-1-hydroxy-13-oxo-3,6,9-trioxa-12-azaheptadecan-17-oic acid (71)

Compound 70 (50 mg, 83 μmol) was dissolved in TFA (2 mL) and stirred for 1 minute at room temperature. Solvent was removed under reduced pressure and the residue was purified by reversed-phase chromatography (C18 column, 0-75% acetonitrile-water with 0.05% TFA). Pure fractions were collected and lyophilized to obtain compound 71 as a white solid (35 mg, 83 μmol, 77% yield). LRMS (ESI): m/z 545.3 [M+H]⁺, Calcd for C₂₈H₃₆N₂O₉ m/z: 545.2.

### Preparation of 4-((14S,19S,22S)-14-amino-1-hydroxy-19-isopropyl-22-methyl-13,17,20-trioxo-3,6,9-trioxa-12,18,21-triazatricosan-23-amido)-3-(((2S,3R,4S,5R,6R)-3,4,5-trihydroxy-6-(hydroxymethyl)tetrahydro-2H-pyran-2-yl)oxy)benzyl (2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamate (72)

To a mixture of amine 30 (30 mg, 27 μmol) and carboxylic acid 71 (15 mg, 28 μmol) in DMF (0.5 mL) were added HATU (10 mg, 27 μmol), followed by DIPEA (14 μL, 82 μmol) at room temperature. Reaction mixture was stirred for 1 h until coupling was found complete by LCMS analysis. Solvent was removed under reduced pressure; the residue was dissolved in MeOH (1 mL) and treated with 1M aqueous LiOH solution (1 mL) at 0° C. Reaction mixture was allowed to slowly warm up to room temperature, stirred for additional 1 h, and quenched with pH 4.7 acetate buffer (1 mL). Solids were filtered off, and the clear filtrate was purified by reversed-phase prep HPLC (C18 column, 0-75% acetonitrile-water with 0.05% TFA). Pure fractions were combined and lyophilized to give 30 mg (24 μmol, 89% yield) of compound 72 as a yellow solid. LRMS (ESI): m/z 1235.5 [M+H]⁺, Calcd for C₆₀H₈₂N₈O₂ m/z: 1235.6.

### Preparation of 4-((14S,19S,22S)-14-(4-((2-((1,2-dimethylhydrazinyl)methyl)-1-((S)-14-(3-(((S)-1-(((S)-1-((4-((((2-((S)-4-ethyl-4-hydroxy-3,14-dioxo-3,4,12,14-tetrahydro-1H-pyrano[3′,4′:6,7]indolizino[1,2-b]quinolin-1-yl)ethyl)(isopropyl)carbamoyl)oxy)methyl)-2-(((2S,3R,4S,5R,6R)-3,4,5-trihydroxy-6-(hydroxymethyl)tetrahydro-2H-pyran-2-yl)oxy)phenyl)amino)-1-oxopropan-2-yl)amino)-3-methyl-1-oxobutan-2-yl)amino)-3-oxopropyl)-1-hydroxy-13,16-dioxo-3,6,9-trioxa-12,15-diazaoctadecan-18-yl)-1H-indol-5-yl)amino)-4-oxobutanamido)-1-hydroxy-19-isopropyl-22-methyl-13,17,20-trioxo-3,6,9-trioxa-12,18,21-triazatricosan-23-amido)-3-(((2S,3R,4S,5R,6R)-3,4,5-trihydroxy-6-(hydroxymethyl)tetrahydro-2H-pyran-2-yl)oxy)benzyl (2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamate (73)

To a solution of compound 72 (30 mg, 24 μmol) in DMF (1.0 mL) were added DIPEA (13 μL, 73 μmol) and HOAt (4.2 mg, 32 μmol), followed by bis-PFP ester 11 (11 mg, 12 mol) in one portion at room temperature. Reaction mixture was allowed to stand for 1 h until reaction was judged complete by LCMS analysis, and treated with piperidine (49 μL, 0.49 mmol) at room temperature. Reaction mixture was directly purified by reversed-phase prep HPLC (C₁₈ column, 0-70% acetonitrile-water with 0.05% TFA). Pure fractions were collected and lyophilized to give 24 mg of compound 73 as a yellow solid (8.5 μmol, 70% yield). LRMS (ESI): m/z 1406.3 [M+2H]²⁺, Calcd for C₁₃₈H₁₈₄N₂₀O₄₃ m/z: 1406.2.

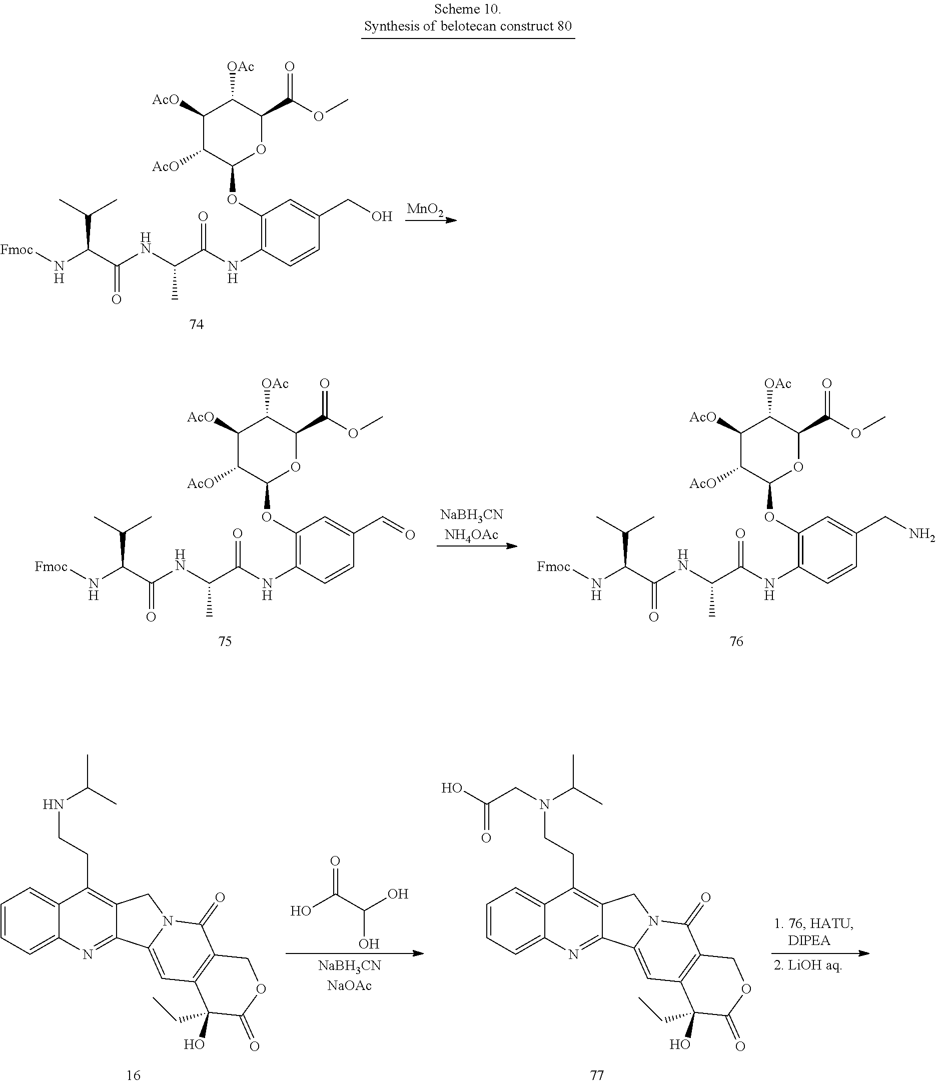

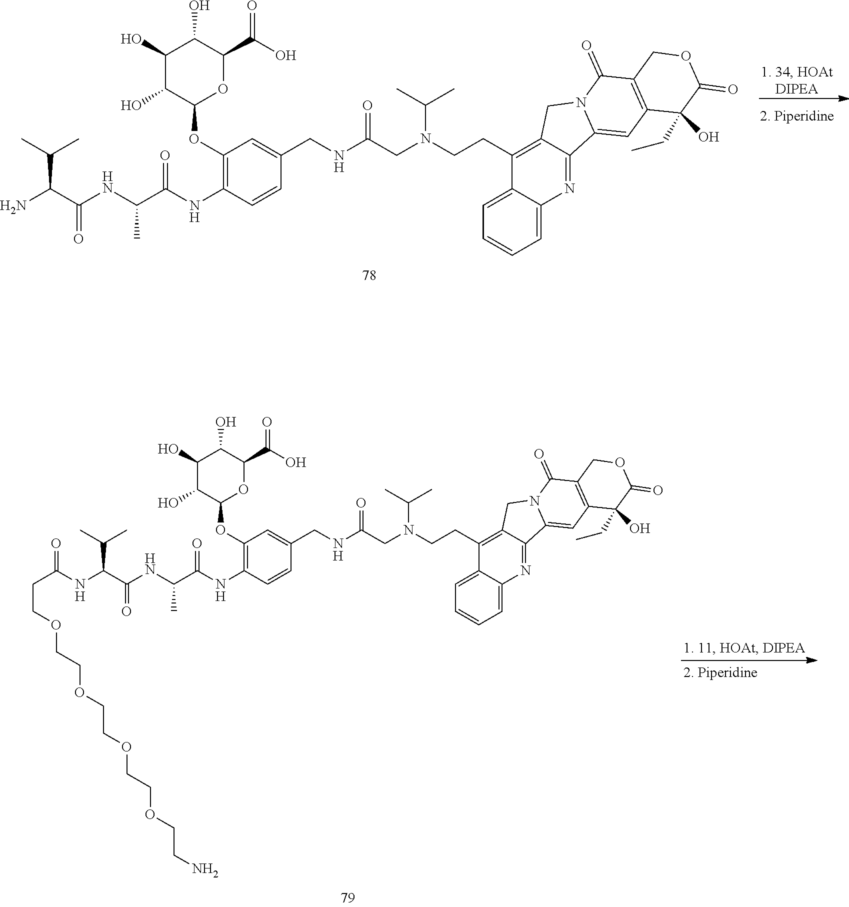

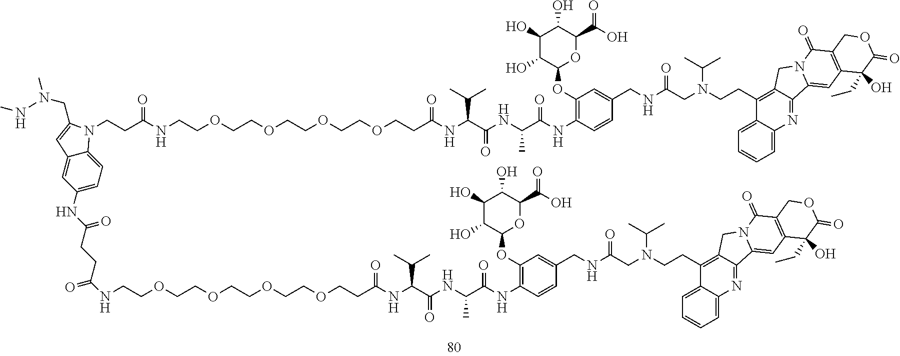 [SMILES: CC[C@@]1(O)C(=O)OCc2c1cc1n(c2=O)Cc2c-1nc1ccccc1c2CCN(CC(=O)NCc1ccc(NC(=O)[C@H](C)NC(=O)[C@@H](NC(=O)CCOCCOCCOCCOCCNC(=O)CCC(=O)Nc2ccc3c(c2)cc(CN(C)NC)n3CCC(=O)NCCOCCOCCOCCOCCC(=O)N[C@H](C(=O)N[C@@H](C)C(=O)Nc2ccc(CNC(=O)CN(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)c(O[C@@H]2O[C@H](C(=O)O)[C@@H](O)[C@H](O)[C@H]2O)c1)C(C)C]

### Preparation of (2S,3R,4S,5S,6S)-2-(2-((S)-2-((S)-2-((((9H-fluoren-9-yl)methoxy)carbonyl)amino)-3-methylbutanamido)propanamido)-5-formylphenoxy)-6-(methoxycarbonyl)tetrahydro-2H-pyran-3,4,5-triyl triacetate (75)

To a round-bottom flask with a stir bar were added alcohol 74 (0.075 g, 0.088 mmol) and anhydrous DCM (15 mL), followed by MnO₂ at ambient temperature in one portion (0.400 g, 4.6 mmol, activated by heating overnight in an oven @ 130° C.). Reaction mixture was allowed to stir for 90 minutes, until starting material was completely consumed as judged by TLC analysis. Reaction mixture was filtered through a pad of celite, eluted with DCM. Combined filtrates were concentrated and purified by silica gel chromatography (0-50% gradient of EtOAc-hexane) to give aldehyde 75 as a white solid (0.057 g, 0.068 mmol, 77% yield). LRMS (ESI): m/z 846.5 [M+H]⁺, Calcd for C₄₃H₄₇N₃O₁₅ m/z: 846.3.

### Preparation of (2S,3R,4S,5S,6S)-2-(2-((S)-2-((S)-2-((((9H-fluoren-9-yl)methoxy)carbonyl)amino)-3-methylbutanamido)propanamido)-5-(aminomethyl)phenoxy)-6-(methoxycarbonyl)tetrahydro-2H-pyran-3,4,5-triyl triacetate (76)

To an oven dried vial with a stir bar were added aldehyde 75 (0.100 g; 0.118 mmol) and anhydrous MeOH (10 mL), followed by oven-dried 4 A molecular sieves (˜1 g). The resulting mixture was allowed to stir for 10 min at room temperature. Anhydrous ammonium acetate (0.911 g; 11.8 mmol) was then added to the mixture and stirring continued for 1 h before the addition of sodium cyanoborohydride (0.038 g; 0.591 mmol) in one portion at room temperature. After stirring for additional 1 h, reaction mixture was filtered, concentrated under reduced pressure, and purified by silica gel chromatography (0-10% MeOH in DCM gradient) to give 0.043 g of amine product 76 (0.051 mmol, 43% yield). LRMS (ESI): m/z 847.4 [M+H]⁺, Calcd for C₄₃H₅₀N₄O₁₄ m/z: 847.3.

### Preparation of (S)-N-(2-(4-ethyl-4-hydroxy-3,14-dioxo-3,4,12,14-tetrahydro-1H-pyrano[3′,4′:6,7]indolizino[1,2-b]quinolin-11-yl)ethyl)-N-isopropylglycine (77)

Belotecan-HCl (16, 0.025 g; 0.057 mmol) was dissolved in DMF (0.25 mL) and diluted with MeOH (3.0 mL). The resulting solution was combined with glyoxylic acid (0.011 g; 0.115 mmol) and sodium acetate (0.033 g; 0.40 mmol) and stirred for 1 h at room temperature. Reaction mixture was then treated with sodium cyanoborohydride (0.025 g; 0.40 mmol), stirred overnight at room temperature, and quenched with 1 mL of 0.05% aqueous TFA. Solvents were removed in vacuum to leave crude oil, which was purified by reversed-phase prep HPLC (C₁₈ column, 5-55% acetonitrile-water/0.05% TFA). Fractions containing the desired product were collected and lyophilized to give 0.027 g (0.055 mmol, 96% yield) of compound 77 as a pale-yellow solid. LRMS (ESI): m/z 492.2 [M+H]⁺, Calcd for C₂₇H₂₉N₃O₆ m/z: 492.2.

### Preparation of (2S,3S,4S,5R,6S)-6-(2-((S)-2-((S)-2-amino-3-methylbutanamido)propanamido)-5-((2-((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)amino)acetamido)methyl)phenoxy)-3,4,5-trihydroxytetrahydro-2H-pyran-2-carboxylic acid (78)

To an oven-dried scintillation vial with a stir bar were added carboxylic acid 77 (0.018 g; 0.037 mmol) and anhydrous DMF (2 mL), followed by HATU (0.013 g; 0.034 mmol) and DIPEA (30 μL) at room temperature. The mixture was allowed to stir for 45 min and then combined with a mixture amine 76 (0.026 g, 0.030 mmol) and DIPEA (30 μL) in 2 mL of DMF. Reaction mixture was stirred for 1 h, quenched by addition of aqueous 1% TFA solution (15 mL), transferred to a separatory funnel, and extracted with EtOAc. Organic layer was washed with water and brine, and dried over Na₂SO₄. Removal of solvents under vacuum gave a crude yellow oily solid (0.048 g), which was dissolved in 5 mL of THF. This solution was cooled to 0° C. in an ice bath and slowly treated with chilled aqueous LiOH (1M, 2.0 mL). Reaction mixture was allowed to stir at 0° C. for 1 h, slowly warmed to room temperature, and quenched by adding aqueous HCl (1.0 M) to pH 4. The mixture was purified by reversed-phase prep HPLC (C18 column, 0-50% acetonitrile-water/0.05% TFA) to give 0.020 g of compound 78 (0.021 mmol, 70% yield) as an off-white solid. LRMS (ESI): m/z 959.1 [M+H]⁺, Calcd for C₄₈H₅₉N₇O₁₄ m/z: 958.4

### (2S,3S,4S,5R,6S)-6-(2-((17S,20S)-1-amino-17-isopropyl-20-methyl-15,18-dioxo-3,6,9,12-tetraoxa-16,19-diazahenicosan-21-amido)-5-((2-((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)amino)acetamido)methyl)phenoxy)-3,4,5-trihydroxytetrahydro-2H-pyran-2-carboxylic acid (79)

To a solution of amine 78 (0.020 g, 0.021 mmol) in anhydrous DMF (2 mL) were added PFP ester 34 (0.020 g, 0.031 mmol), HOAt (0.004 g; 0.031 mmol), and DIPEA (11 μL) at room temperature. Reaction mixture was allowed to stir for 45 min, then treated with piperidine (50 μL) and stirred for additional 20 min. The mixture was purified by reversed-phase prep HPLC (C18 column, 0-50% acetonitrile-water/0.05% TFA). Pure fractions were combined and lyophilized to obtain amine product 79 as a pale-yellow solid (0.015 g, 0.012 mmol, 57% yield). LRMS (ESI): m/z 1205.5 [M+H]⁺, Calcd for C₅₉H₈₀N₈O₁₉ m/z: 1205.6.

### (2S,3S,4S,5R,6S)-6-(2-((2S,5S)-25-(5-((2S,5S)-1-((2-(((2S,3R,4S,5S,6S)-6-carboxy-3,4,5-trihydroxytetrahydro-2H-pyran-2-yl)oxy)-4-((2-((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)amino)acetamido)methyl)phenyl)amino)-5-isopropyl-2-methyl-1,4,7,23-tetraoxo-10,13,16,19-tetraoxa-3,6,22-triazahexacosan-26-amido)-2-((1,2-dimethylhydrazinyl)methyl)-1H-indol-1-yl)-5-isopropyl-2-methyl-4,7,23-trioxo-10,13,16,19-tetraoxa-3,6,22-triazapentacosanamido)-5-((2-((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)amino)acetamido)methyl)phenoxy)-3,4,5-trihydroxytetrahydro-2H-pyran-2-carboxylic acid (80)

To a solution of amine 79 (15 mg; 12 μmol) in anhydrous DMF (2 mL) were added bis-PFP ester 11 (5.5 mg; 6 μmol), followed by HOAt (3.4 mg; 2.5 μmol) and DIPEA (22 μL) at room temperature. The resulting mixture was allowed to stir for 30 min, then 50 μL of piperidine was added, and stirring continued for 20 min. Reaction mixture was diluted with 0.05% TFA (1 mL) and purified by reversed-phase prep HPLC (C18 column, 0-50% acetonitrile-water/0.05% TFA). Pure fractions were collected and immediately subjected to lyophilization to give 5.2 mg of compound 80 as a yellow solid (1.9 μmol, 32% yield). LRMS (ESI): m/z 1376.2 [M+2H]²⁺, Calcd for C₁₃₆H₁₈₀N₂₀O₄₁ m/z: 1376.1.

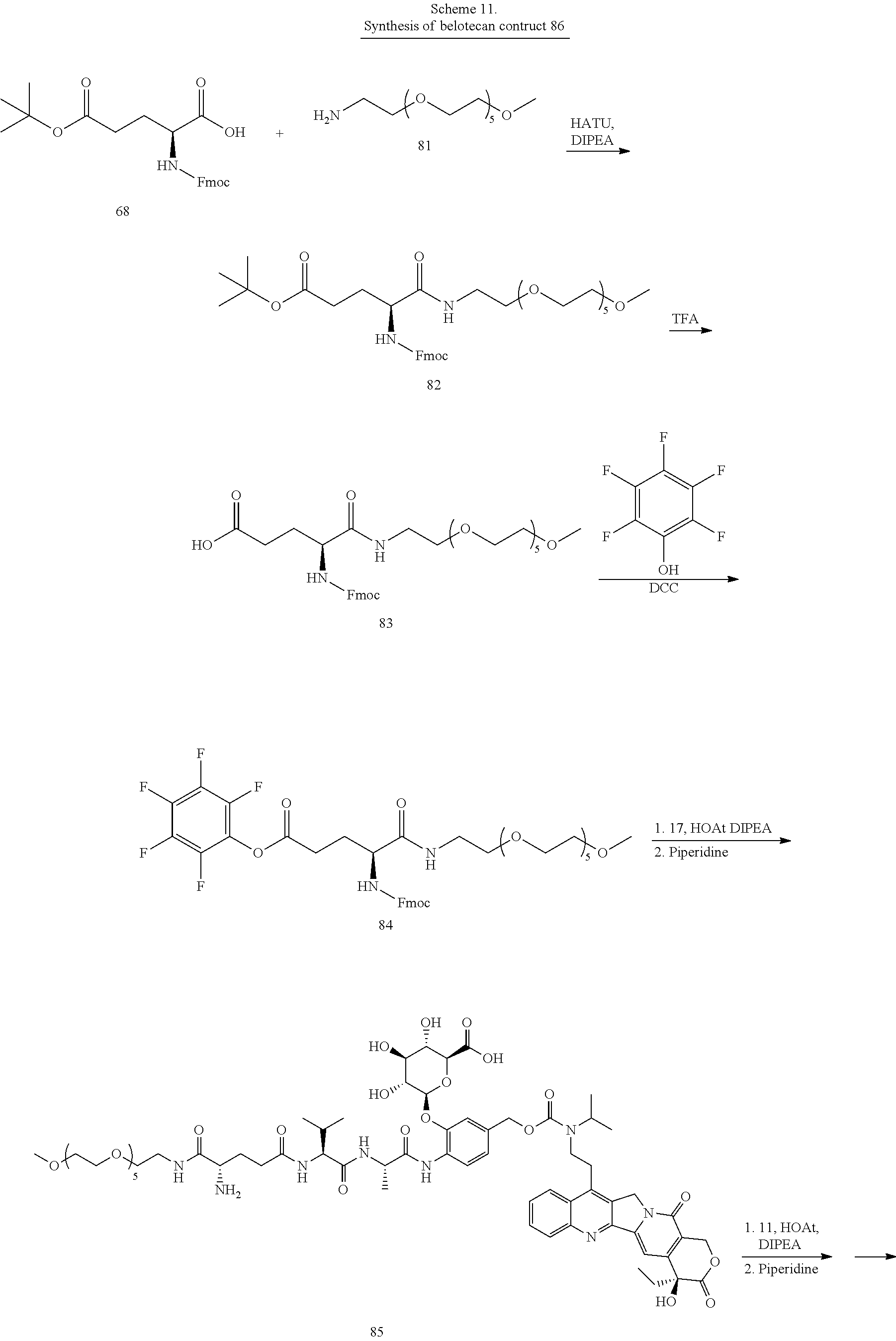

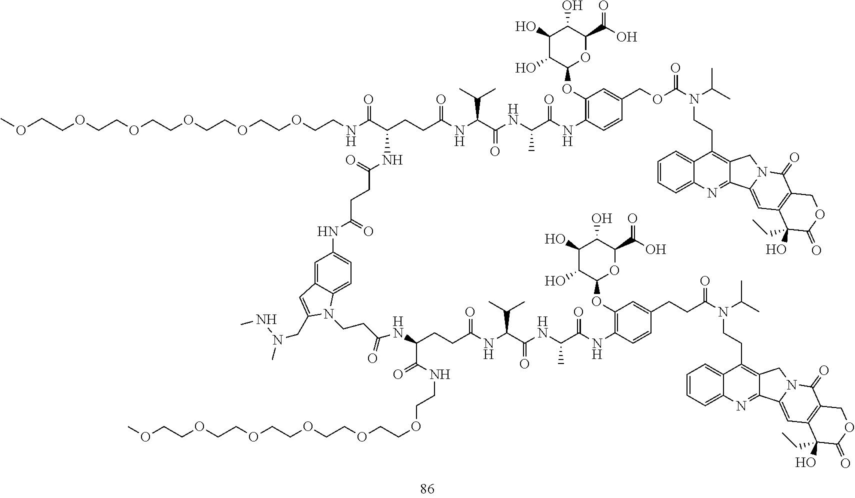 [SMILES: CC[C@@]1(O)C(=O)OCc2c1cc1n(c2=O)Cc2c-1nc1ccccc1c2CCN(C(=O)CCc1ccc(NC(=O)[C@H](C)NC(=O)[C@@H](NC(=O)CC[C@H](NC(=O)CCn2c(CN(C)NC)cc3cc(NC(=O)CCC(=O)N[C@@H](CCC(=O)N[C@H](C(=O)N[C@@H](C)C(=O)Nc4ccc(COC(=O)N(CCc5c6c(nc7ccccc57)-c5cc7c(c(=O)n5C6)COC(=O)[C@]7(O)CC)C(C)C)cc4O[C@@H]4O[C@H](C(=O)O)[C@@H](O)[C@H](O)[C@H]4O)C(C)C)C(=O)NCCOCCOCCOCCOCCOCCOC)ccc32)C(=O)NCCOCCOCCOCCOCCOCCOC)C(C)C)c(O[C@@H]2O[C@H](C(=O)O)[C@@H](O)[C@H](O)[C@H]2O)c1)C(C)C]

### Preparation of tert-butyl (S)-22-((((9H-fluoren-9-yl)methoxy)carbonyl)amino)-21-oxo-2,5,8,11,14,17-hexaoxa-20-azapentacosan-25-oate (82)

To a round bottom flask with a stir bar were added Fmoc-Glu(OtBu)-OH 68 (0.259 g; 0.609 mmol) and DMF (15 mL), followed by HATU (0.215 g; 0.558 mmol) and DIPEA (440 μL; 2.54 mmol) at room temperature. The resulting mixture was allowed to stir for 30 min, and combined with mPEG6-amine 81 (0.150 g, 0.507 mmol). After 1 h, reaction mixture was transferred to a separatory funnel, diluted with water (30 mL), and extracted with EtOAc (2×30 mL). Organic layer was washed with water and brine, dried over sodium sulfate. Solvents were removed in vacuum to give 0.50 g of crude product 82 as a colorless oil, which was used further without purification. LRMS (ESI): m/z 703.4 [M+H]⁺, Calcd for C₃₇H₅₄N₂O₁₁ m/z: 703.4.

### Preparation of (S)-22-((((9H-fluoren-9-yl)methoxy)carbonyl)amino)-21-oxo-2,5,8,11,14,17-hexaoxa-20-azapentacosan-25-oic acid (83)

Crude compound 82 (0.50 g) was dissolved in anhydrous DCM (5 mL) and treated with TFA (2 mL) at room temperature. Reaction mixture was allowed to stir for 2 h, then solvents were removed under reduced pressure, and the residue was dried under high vacuum overnight to give 0.50 g of crude carboxylic acid 83 as a colorless oil, which was used further without purification. LRMS (ESI): m/z 647.7 [M+H]⁺, Calcd for C₃₃H₄₆N₂O₁₁ m/z: 647.3.

### Preparation of perfluorophenyl (S)-22-((((9H-fluoren-9-yl)methoxy)carbonyl)amino)-21-oxo-2,5,8,11,14,17-hexaoxa-20-azapentacosan-25-oate (84)

To a stirred solution of crude carboxylic acid 83 (0.50 g) in anhydrous THF (20 mL) were added pentafluorophenol (1.42 g; 7.73 mmol), followed by DCC (0.32 g; 1.55 mmol) in one portion at room temperature. Reaction mixture was stirred overnight at room temperature, filtered, and concentrated under vacuum. The residue was then purified by silica gel chromatography (0-10% MeOH in DCM gradient) to give PFP-ester 84 as a colorless solid (0.43 g, 0.53 mmol, 87% yield over 3 steps). LRMS (ESI): m/z 813.7 [M+H]⁺, Calcd for C₃₉H₄₅F₅N₂O₁₁ m/z: 813.3.

### Preparation of (2S,3S,4S,5R,6S)-6-(2-((22S,27S,30S)-22-amino-27-isopropyl-30-methyl-21,25,28-trioxo-2,5,8,11,14,17-hexaoxa-20,26,29-triazahentriacontan-31-amido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (85)

To a solution of compound 17 (30 mg, 31 μmol) in anhydrous DMF (3 mL) were added PFP-ester 84 (31 mg, 38 μmol), followed by HOAt (1.5 mg, 47 μmol) and DIPEA (10 μL) at room temperature. Reaction mixture was stirred for 45 min, then directly treated with piperidine (50 μL). After 30 min, reaction mixture was quenched with aqueous 0.05% TFA (1 mL) and purified by reversed-phase prep HPLC (C18 column, 0-50% acetonitrile-water/0.05% TFA). Fractions containing the desired product were combined and lyophilized to yield 38 mg of amine 85 as a pale-yellow solid (28 μmol, 90% yield). LRMS (ESI): m/z 1351.6 [M+H]⁺, Calcd for C₆₅H₉₀N₈O₂₃ m/z: 1351.6.

### (2S,3S,4S,5R,6S)-6-(2-((22S,27S,30S)-22-(3-(5-((S)-22-(3-(((S)-1-(((S)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-1-yl)ethyl)(isopropyl)carbamoyl)oxy)methyl)phenyl)amino)-1-oxopropan-2-yl)amino)-3-methyl-1-oxobutan-2-yl)amino)-3-oxopropyl)-21,24-dioxo-2,5,8,11,14,17-hexaoxa-20,23-diazaheptacosan-27-amido)-2-((1,2-dimethylhydrazinyl)methyl)-1H-indol-1-yl)propanamido)-27-isopropyl-30-methyl-21,25,28-trioxo-2,5,8,11,14,17-hexaoxa-20,26,29-triazahentriacontan-31-amido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (86)

To a stirred solution of amine 85 (20 mg; 15 μmol) in 3 mL of anhydrous DMF were added bis-PFP ester 11 (6.8 mg; 7.3 μmol), followed by HOAt (2.5 mg; 18 μmol) and DIPEA (13 μL) at room temperature. Reaction mixture was stirred for 30 min and then treated directly with piperidine (50 μL). After 20 min, reaction mixture was purified by reversed-phase prep HPLC (C18 column, 0-50% acetonitrile-water/0.05% TFA). Pure fractions containing product were combined and lyophilized to yield 15 mg of compound 86 (5 μmol, 69% yield) as a pale-yellow solid. LRMS (ESI): m/z 1522.2 [M+2H]²⁺, Calcd for C₁₄₈H₂₀₀N₂₀O₄₉ m/z: 1522.2.

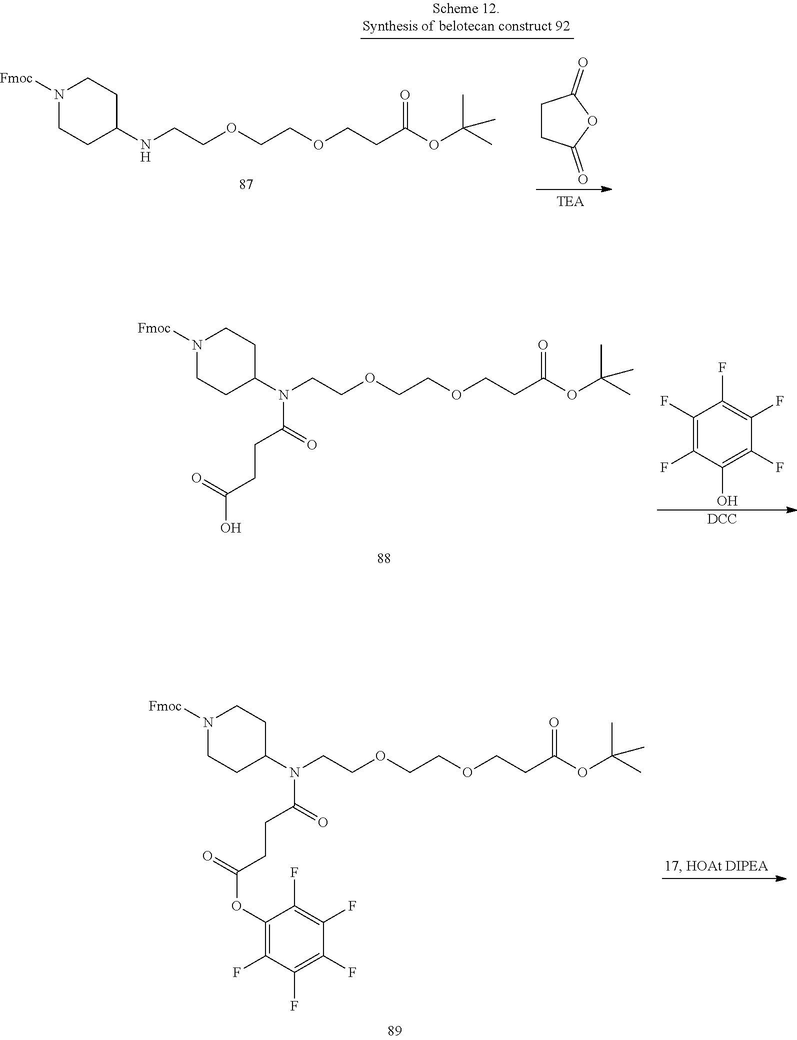

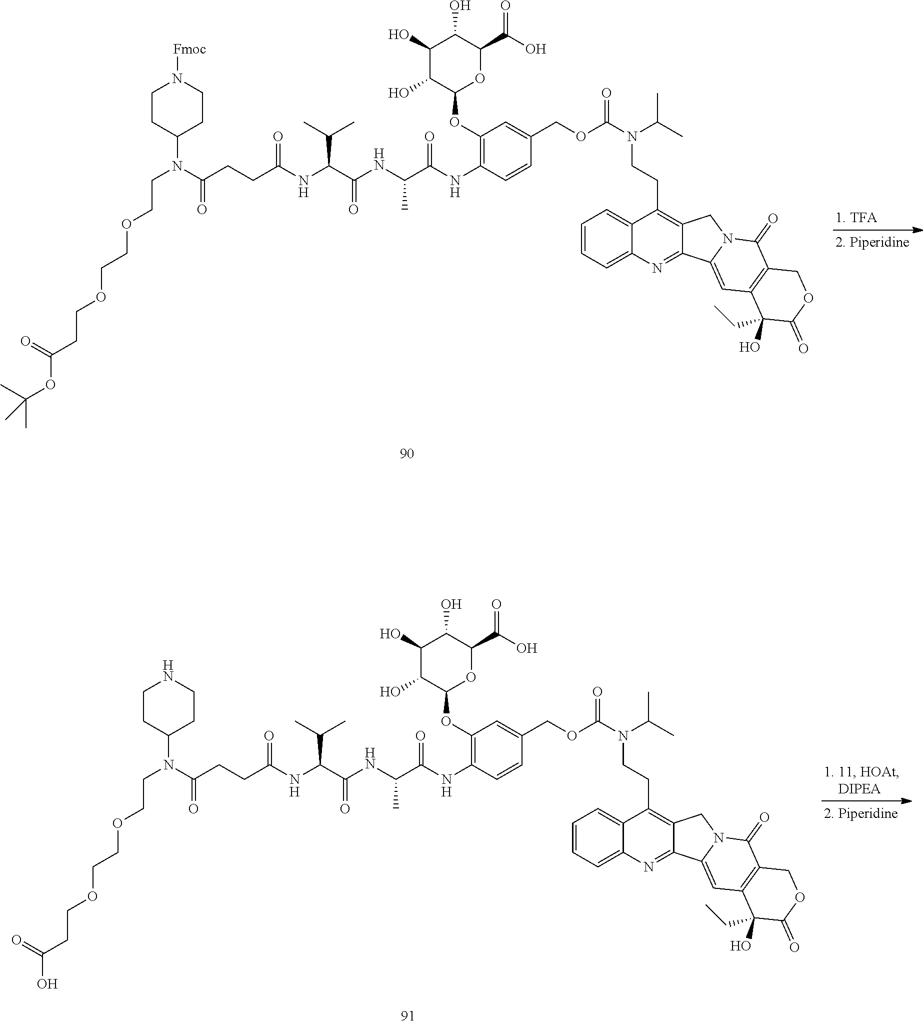

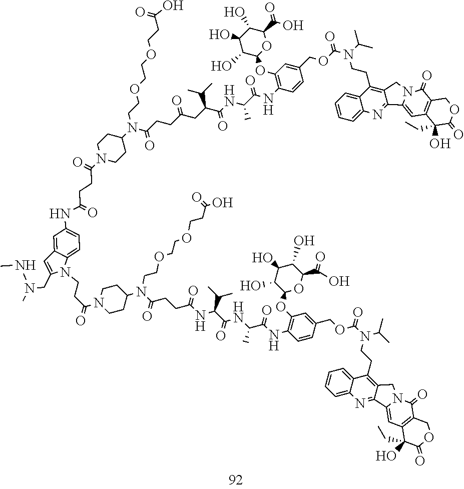 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](CC(=O)CCC(=O)N(CCOCCOCCC(=O)O)C2CCN(C(=O)CCC(=O)Nc3ccc4c(c3)cc(CN(C)NC)n4CCC(=O)N3CCC(N(CCOCCOCCC(=O)O)C(=O)CCC(=O)N[C@H](C(=O)N[C@@H](C)C(=O)Nc4ccc(COC(=O)N(CCc5c6c(nc7ccccc57)-c5cc7c(c(=O)n5C6)COC(=O)[C@]7(O)CC)C(C)C)cc4O[C@@H]4O[C@H](C(=O)O)[C@@H](O)[C@H](O)[C@H]4O)C(C)C)CC3)CC2)C(C)C)c(O[C@@H]2O[C@H](C(=O)O)[C@@H](O)[C@H](O)[C@H]2O)c1)C(C)C]

### Preparation of 13-(1-(((9H-fluoren-9-yl)methoxy)carbonyl)piperidin-4-yl)-2,2-dimethyl-4,14-dioxo-3,7,10-trioxa-13-azaheptadecan-17-oic acid (88)

To a stirred solution of compound 87 (100 mg, 186 μmol) in MeCN (2 mL) were added succinic anhydride (93 mg, 928 μmol) and triethylamine (129 μL, 928 μmol) at ambient temperature. Reaction mixture was stirred for 10 min and then directly purified by reversed-phase chromatography (C18 column, 0-50% acetonitrile-water/0.05% TFA). Pure fractions were collected and lyophilized to obtain compound 88 as a colorless oil (90 mg, 141 μmol, 76% yield).

### Preparation of (9H-fluoren-9-yl)methyl 4-(N-(2-(2-(3-(tert-butoxy)-3-oxopropoxy)ethoxy)ethyl)-4-oxo-4-(perfluorophenoxy)butanamido)piperidine-1-carboxylate (89)

To a mixture of carboxylic acid 88 (90 mg, 141 μmol) and pentafluorophenol (91 mg, 493 μmol) in 2 mL of anhydrous THF were added DCC (101 mg, 493 μmol) at room temperature. Reaction mixture was stirred overnight, solids were filtered off, solvent was removed under reduced pressure, and the residue was purified by silica gel chromatography (EtOAc-hexane, 0-50% gradient) to yield 42 mg of PFP-ester 89 (52 μmol, 37% yield) as an off-white solid.

### Preparation of (2S,3S,4S,5R,6S)-6-(2-((2S,5S)-11-(1-(((9H-fluoren-9-yl)methoxy)carbonyl)piperidin-4-yl)-5-isopropyl-2,22,22-trimethyl-4,7,10,20-tetraoxo-14,17,21-trioxa-3,6,11-triazatricosanamido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (90)

To a solution of compound 17 (25 mg, 26 μmol) in DMF (1.0 mL) were added DIPEA (14 μL, 73 μmol) and HOAt (5 mg, 35 μmol), followed by PFP-ester 89 (21 mg, 26 mol) at room temperature. Reaction mixture was stirred for 30 min and then directly purified by reversed-phase chromatography (C18, 0-100% v/v MeCN—H₂O with 0.05% TFA). Lyophilized pure fractions gave 38 mg of compound 90 (24 μmol, 92% yield) as a yellow powder. LRMS (ESI): m/z 1565.7 [M+H]⁺, Calcd for C₈₂H₁₀₀N₈O₂₃ m/z: 1565.7.

### Preparation of (2S,5S)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-5-isopropyl-2-methyl-1,4,7,10-tetraoxo-11-(piperidin-4-yl)-14,17-dioxa-3,6,11-triazaicosan-20-oic acid (91)

A solution of compound 90 (38 mg, 24 μmol) in TFA (2 mL) was stirred for one minute, then diluted with 2 mL of water-acetonitrile mixture (1:1 v/v) and lyophilized to give a white solid. The solid was dissolved in DMF (1 mL) and treated with piperidine (49 μL, 0.49 mmol) at room temperature. After 20 minutes, reaction mixture was purified by reversed-phase prep HPLC (C18, 0-70% v/v MeCN—H₂O with 0.05% TFA). Lyophilized pure fractions gave 10 mg of compound 91 (7 μmol, 34% yield) as a yellow powder. LRMS (ESI): m/z 1287.5 [M+H]⁺, Calcd for C₆₃H₈₂N₈O₂₁ m/z 1287.6.

### Preparation of (2S,3S,4S,5R,6S)-6-(2-((15S,18S)-1-carboxy-9-(1-(3-(5-(4-(4-(4-(((S)-1-(((S)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1-oxopropan-2-yl)amino)-3-methyl-1-oxobutan-2-yl)amino)-N-(2-(2-(2-carboxyethoxy)ethoxy)ethyl)-4-oxobutanamido)piperidin-1-yl)-4-oxobutanamido)-2-((1,2-dimethylhydrazinyl)methyl)-1H-indol-1-yl)propanoyl)piperidin-4-yl)-15-isopropyl-18-methyl-10,13,16-trioxo-3,6-dioxa-9,14,17-triazanonadecan-19-amido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (92)

To a solution of amine 91 (10 mg, 8 μmol) in anhydrous DMF (0.5 mL) were added DIPEA (2 μL, 12 μmol) and HOAt (0.7 mg, 5 μmol), followed by compound 11 (3.5 mg, 4 mol) in one portion at room temperature. Reaction mixture was stirred for 1 h and then directly treated with piperidine (8 μL, 160 μmol) at room temperature. After 20 minutes, reaction mixture was purified by reversed-phase prep HPLC (C18, 0-70% v/v MeCN—H₂O with 0.05% TFA). Lyophilized pure fractions gave 2.8 mg of compound 92 (1 μmol, 26% yield) as a yellow powder. LRMS (ESI): m/z 1458.2 [M+2H]²⁺, Calcd for C₁₄4H₁₈₄N₂₀O₄₅ m/z 1458.1.

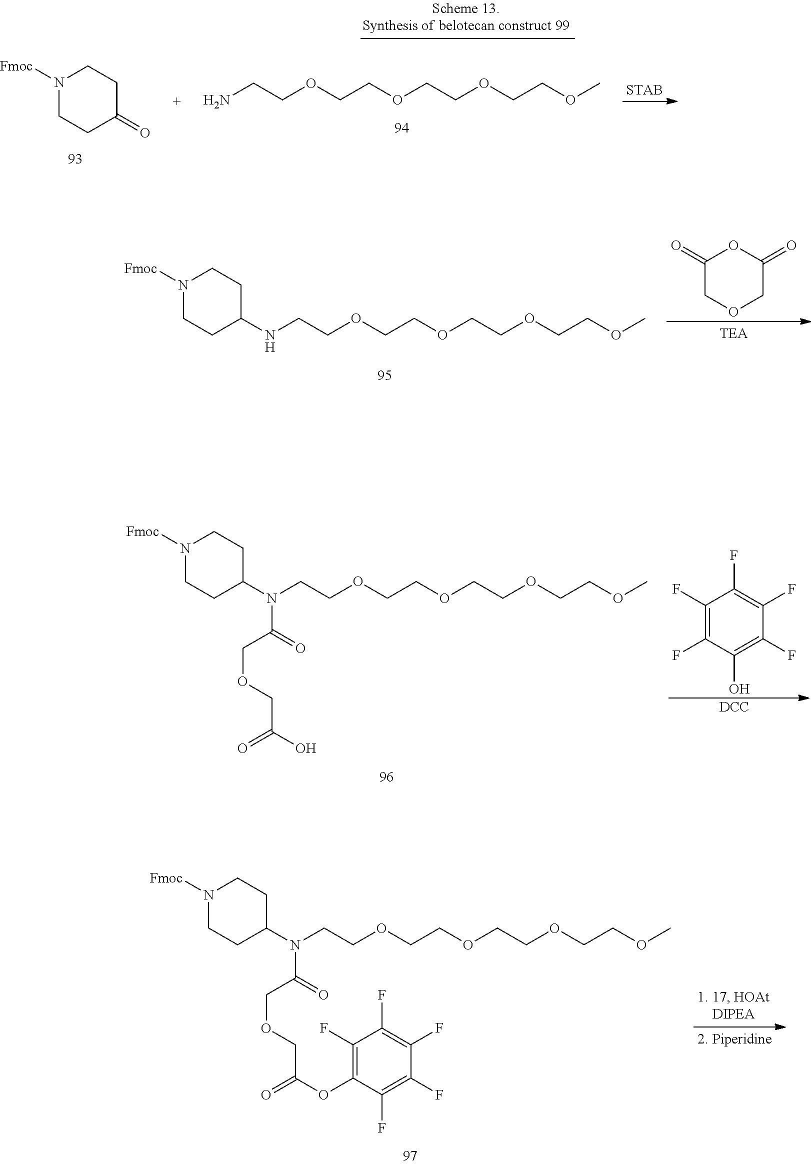

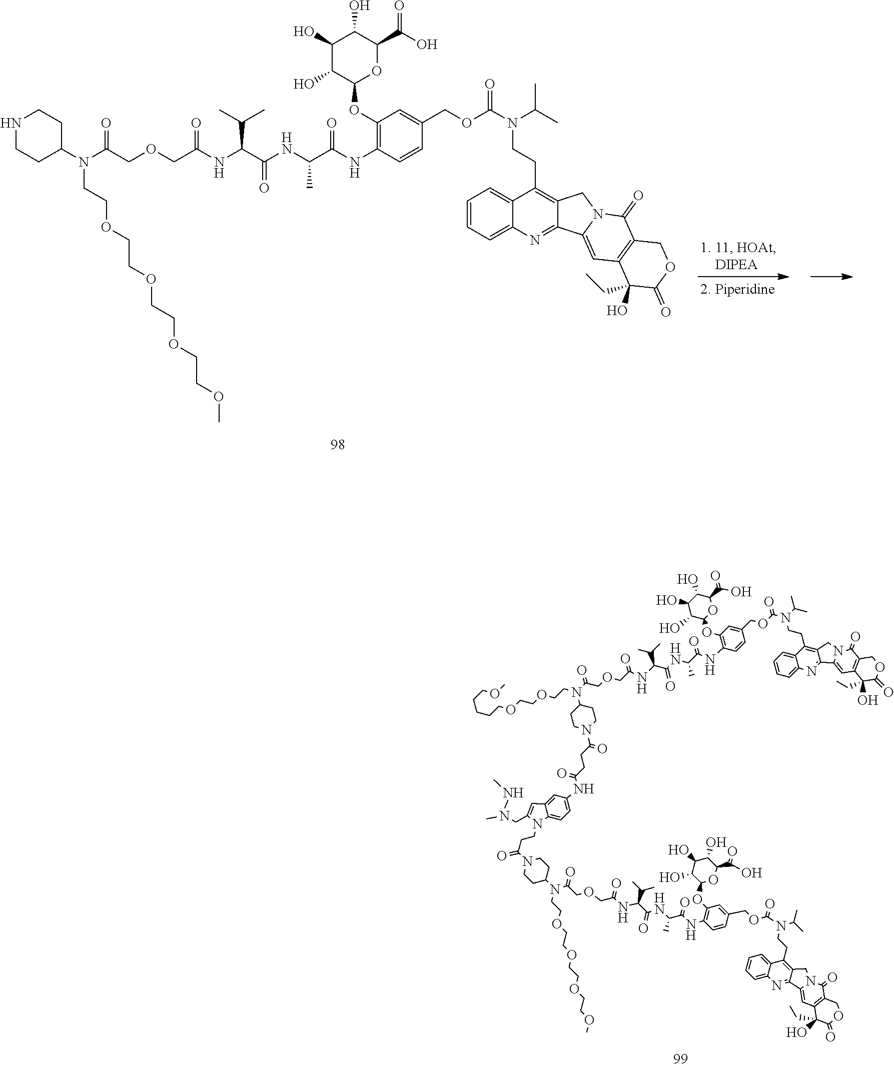

### Preparation of (9H-fluoren-9-yl)methyl 4-((2,5,8,11-tetraoxatridecan-13-yl)amino)piperidine-1-carboxylate (95)

A mixture of N-Fmoc-piperidone (93, 642 mg, 2 mmol) and mPEG4-amine (94, 414 mg, 2 mmol) in DCE (10 mL) was stirred for 30 mins at room temperature, and then treated with STAB (840 mg, 4 mmol) in small portions. The resulting mixture was allowed to stir for 2 h, quenched with sat. sodium bicarbonate solution (5 mL) and extracted with EtOAc (3×15 mL). Combined organic layers were washed with brine and dried over sodium sulfate. Solvents were removed in vacuum to give crude product 95 as colorless oil (900 mg), which was used further without purification.

### Preparation of 14-(1-(((9H-fluoren-9-yl)methoxy)carbonyl)piperidin-4-yl)-15-oxo-2,5,8,11,17-pentaoxa-14-azanonadecan-19-oic acid (96)

To a solution of crude compound 95 (900 mg) in anhydrous MeCN (10 mL) were added 1,4-dioxane-2,6-dione (1.0 g, 0.93 mmol) and triethylamine (0.85 mL, 0.93 mmol) at room temperature. Reaction mixture was stirred for 30 min and then directly purified by reversed-phase chromatography (C18, 0-70% v/v MeCN—H₂O with 0.05% TFA). Pure fractions were collected and lyophilized to obtain compound 96 as a colorless oil (260 mg, 0.45 mmol, 23% yield over 2 steps). LRMS (ESI): m/z 629.3 [M+H]⁺, Calcd for C₃₃H₄₄N₂O₁₀ m/z 629.3.

### Preparation of (9H-fluoren-9-yl)methyl 4-(2-(2-oxo-2-(perfluorophenoxy)ethoxy)-N-(2,5,8,11-tetraoxatridecan-13-yl)acetamido)piperidine-1-carboxylate (97)

To a mixture of acid 96 (260 mg, 0.41 mmol) and pentafluorophenol (264 mg, 1.23 mmol) in 2 mL of anhydrous THF were added DCC (253 mg, 1.23 mmol) at room temperature. Reaction mixture was stirred overnight, solids were filtered off, solvent was removed under reduced pressure, and the residue was purified by silica gel chromatography (EtOAc-hexane 0-50% v/v gradient) to give 163 mg of PFP-ester 97 (0.20 mmol, 50% yield) as a colorless oil. LRMS (ESI): m/z 795.3 [M+H]⁺, Calcd for C₃₉H₄₃F₅N₂O₁₀ m/z 795.3.

### Preparation of (2S,3S,4S,5R,6S)-6-(5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)-2-((21S,24S)-21-isopropyl-24-methyl-15,19,22-trioxo-14-(piperidin-4-yl)-2,5,8,11,17-pentaoxa-14,20,23-triazapentacosan-25-amido)phenoxy)-3,4,5-trihydroxytetrahydro-2H-pyran-2-carboxylic acid (98)

To a solution of compound 17 (25 mg, 26 μmol) in anhydrous DMF (1.0 mL) were added DIPEA (14 μL, 73 μmol) and HOAt (4.6 mg, 34 μmol) followed by PFP-ester 97 (21 mg, 26 μmol) at room temperature. Reaction mixture was stirred for 30 min, then piperidine (52 uL, 0.52 mmol) was added, and stirring continued for 20 minutes. Reaction mixture was purified directly by reversed-phase chromatography (C18, 0-100% v/v MeCN—H₂O with 0.05% TFA). Lyophilized pure fractions gave 22 mg of compound 98 (16 μmol, 62% yield) as a yellow powder. LRMS (ESI): m/z 1333.6 [M+H]+, Calcd for C₆₅H₈₈N₈O₂₂ m/z 1333.6.

### Preparation of (2S,3S,4S,5R,6S)-6-(2-((21S,24S)-14-(1-(4-((1-(3-(4-(2-(2-(((S)-1-(((S)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1-oxopropan-2-yl)amino)-3-methyl-1-oxobutan-2-yl)amino)-2-oxoethoxy)-N-(2,5,8,11-tetraoxatridecan-13-yl)acetamido)piperidin-1-yl)-3-oxopropyl)-2-((1,2-dimethylhydrazinyl)methyl)-1H-indol-5-yl)amino)-4-oxobutanoyl)piperidin-4-yl)-21-isopropyl-24-methyl-15,19,22-trioxo-2,5,8,11,17-pentaoxa-14,20,23-triazapentacosan-25-amido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (99)

To a solution of compound 98 (22 mg, 16 μmol) in anhydrous DMF (1.0 mL) were added DIPEA (4.3 μL, 24 μmol) and HOAt (1.4 mg, 11 μmol), followed by the addition of compound 11 (7 mg, 8 μmol) at room temperature. After 30 minutes, piperidine (16 μL, 0.16 mmol) was added in one shot at room temperature. Reaction mixture was stirred for 15 minutes and then directly purified by reversed-phase prep HPLC (C18, 0-70% v/v MeCN—H₂O with 0.05% TFA). Lyophilized pure fractions gave 15 mg of 99 (5 μmol, 63% yield) as a yellow powder. LRMS (ESI): m/z 1504.2 [M+2H]²⁺, Calcd for C₁₄₈H₁₉₆N₂₀O₄₇ m/z 1504.2.

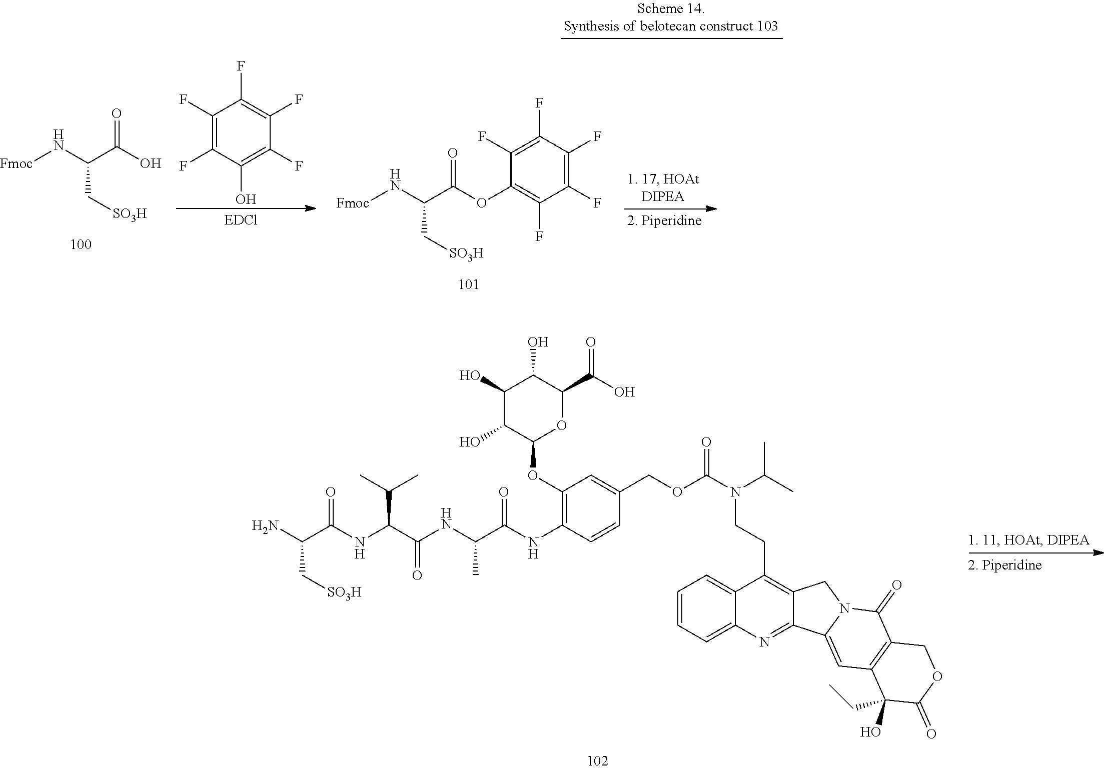

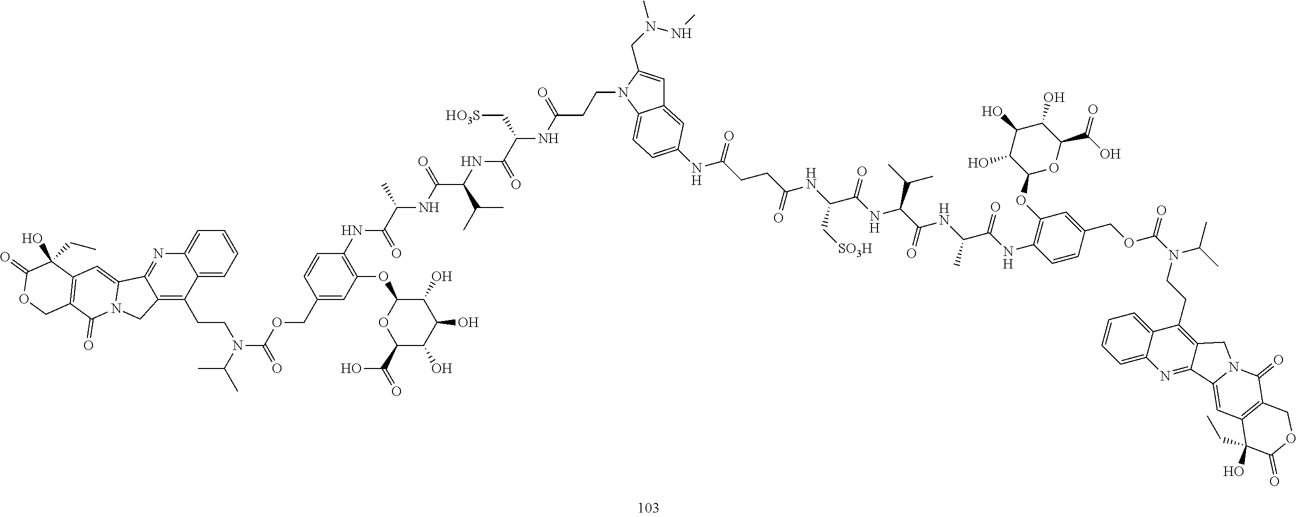 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CS(=O)(=O)O)NC(=O)CCC(=O)Nc2ccc3c(c2)cc(CN(C)NC)n3CCC(=O)N[C@@H](CS(=O)(=O)O)C(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)c(O[C@@H]2O[C@H](C(=O)O)[C@@H](O)[C@H](O)[C@H]2O)c1)C(C)C]

### Preparation of (R)-2-((((9H-fluoren-9-yl)methoxy)carbonyl)amino)-3-oxo-3-(perfluorophenoxy)propane-1-sulfonic acid (101)

To a stirred mixture of Fmoc-L-cysteic acid 100 (100 mg, 0.26 mmol) and pentafluorophenol (94 mg, 0.51 mmol) in 2 mL of anhydrous DMF were added EDCI-HCl (98 mg, 0.51 mmol) in one portion at room temperature. The resulting mixture was stirred overnight and then directly purified by reversed-phase chromatography (C18, 0-100% v/v MeCN—H₂O with 0.05% TFA). Pure fractions were concentrated under reduced pressure until solution became murky and lyophilized to give 122 mg of PFP-ester 101 (0.22 mmol, 85% yield) as an off-white solid. LRMS (ESI−): m/z 556.2 [M−H]⁻, Calcd for C₂₄H₁₆F₅NO₇S m/z 556.1.

### Preparation of (2S,3S,4S,5R,6S)-6-(2-((S)-2-((S)-2-((R)-2-amino-3-sulfopropanamido)-3-methylbutanamido)propanamido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (102)

To a mixture of compound 17 (30 mg, 32 μmol) and DIPEA (11 μL, 64 μmol) in 2 mL of anhydrous DMF were added PFP-ester 101 (18 mg, 32 μmol) at room temperature, followed by HOAt (4.5 mg, 32 μmol). The resulting mixture was allowed to stand at room temperature for 1 h and then treated with piperidine (63 μL, 0.63 mmol). After 20 minutes, reaction mixture was purified by reversed-phase prep HPLC (C18, 0-50% v/v MeCN—H₂O with 0.05% TFA). Pure fractions containing product were combined and lyophilized to give 12 mg of compound 102 (11 μmol, 34% yield) as a yellow solid.

### Preparation of (2S,3S,4S,5R,6S)-6-(2-((S)-2-((S)-2-((R)-2-(4-((1-(3-(((R)-1-(((S)-1-(((S)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1-oxopropan-2-yl)amino)-3-methyl-1-oxobutan-2-yl)amino)-1-oxo-3-sulfopropan-2-yl)amino)-3-oxopropyl)-2-((1,2-dimethylhydrazinyl)methyl)-1H-indol-5-yl)amino)-4-oxobutanamido)-3-sulfopropanamido)-3-methylbutanamido)propanamido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (103)

To a mixture of compound 102 (12 mg, 11 μmol) and DIPEA (4 μL, 22 μmol) in 2 mL of anhydrous DMF were added bis-PFP-ester 11 (4.5 mg, 5 μmol) at room temperature, followed by HOAt (1.5 mg, 11 μmol). The resulting mixture was allowed to stand at room temperature for 1 h and then treated with piperidine (22 μL, 0.22 mmol). After 20 minutes, reaction mixture was purified by reversed-phase prep HPLC (C18, 0-50% v/v MeCN-H₂O/10 mM ammonium formate). Pure fractions containing product were combined and lyophilized to give 7 mg of compound 103 (2.8 μmol, 56% yield) as a tan powder. LRMS (ESI): m/z 1266.5 [M+2H]²⁺, Calcd for C₁₁₈H₁₄₂N₁₈O₄₁S₂ m/z 1266.5.

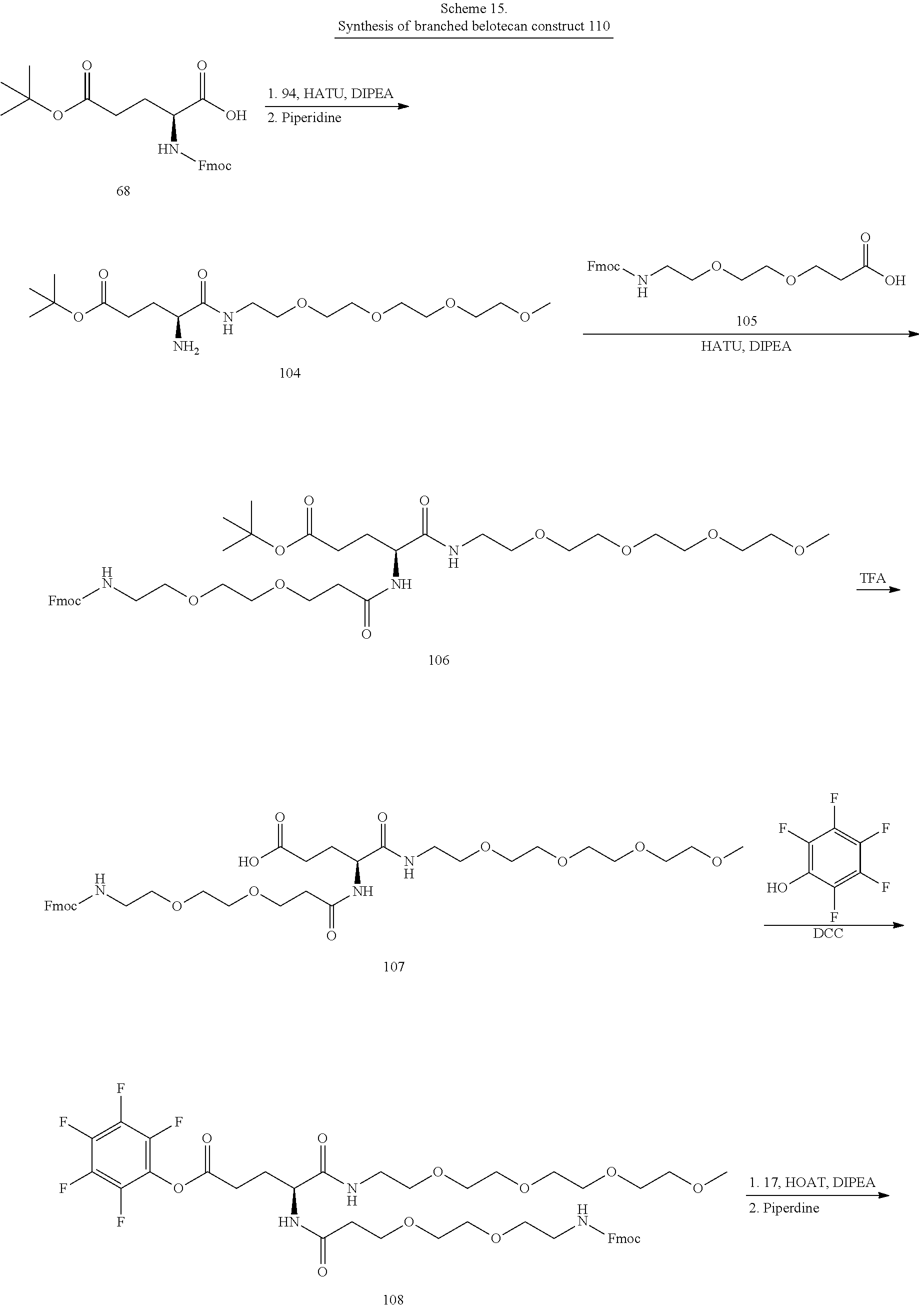

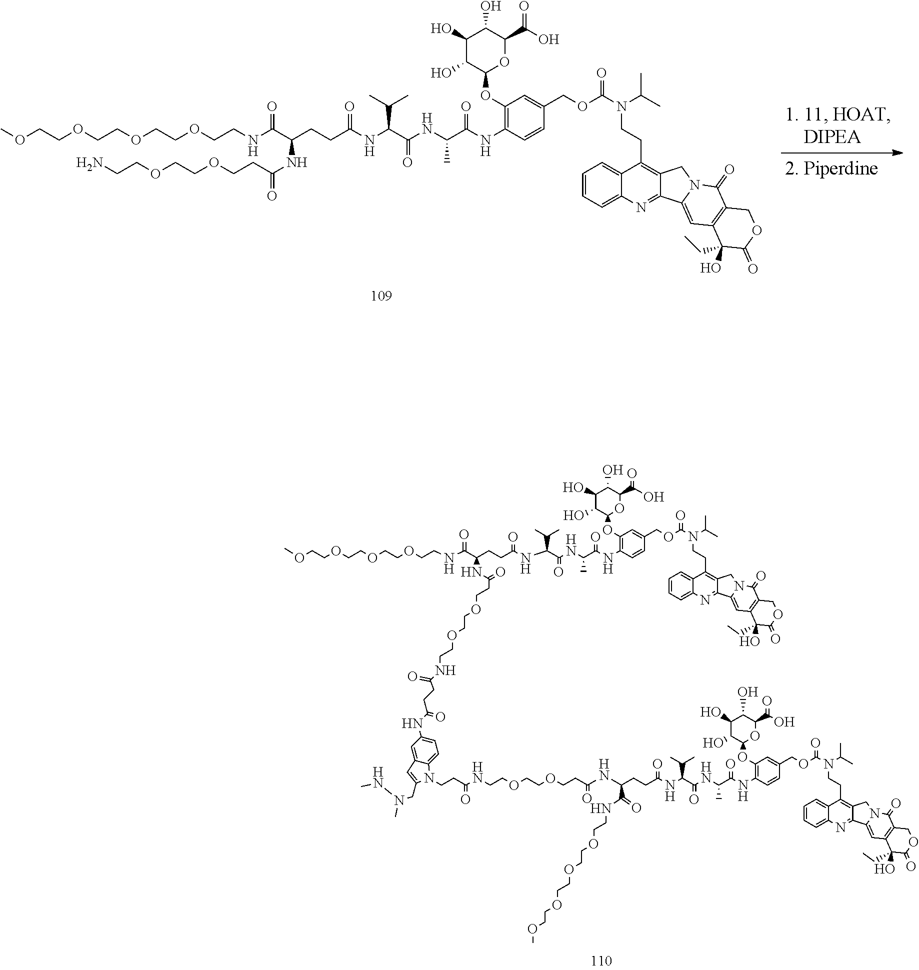

### Preparation of tert-butyl (S)-16-amino-15-oxo-2,5,8,11-tetraoxa-14-azanonadecan-19-oate (104)

To a solution of Fmoc-Glu-OtBu 68 (0.49 g, 1.2 mmol) in DMF (15 mL) were added HATU (0.42 g, 1.1 mmol) and DIPEA (1 mL) at room temperature. The resulting mixture was stirred for 45 min, then combined with mPEG4-amine 94 (0.20 g, 0.96 mmol) and stirred for 30 min at room temperature. Reaction was quenched by addition of 0.05% TFA in water (30 mL) and extracted with EtOAc (2×30 mL). Organic layer was washed with water and brine, dried over Na₂SO₄, filtered, and concentrated in vacuum to give crude oil. The crude was resuspended in acetonitrile (20 mL) and treated with piperidine (1.0 mL, 1 mmol) at room temperature. After 45 min, solvents were removed in vacuum to give crude oil, which was washed once with hexane (10 mL) and purified by reversed-phase chromatography (C18, 0-50% v/v MeCN—H₂O with 0.05% TFA). Pure fractions were combined and concentrated, followed by lyophilization to give amine 104 (0.23 g, 0.57 mmol, 58% yield) as an oily solid. LRMS (ESI): m/z 393.3 [M+H]⁺, Calcd for C₁₈H₃₆N₂O₇ m/z 393.3.

### Preparation of tert-butyl (S)-16-(1-(9H-fluoren-9-yl)-3-oxo-2,7,10-trioxa-4-azatridecan-13-amido)-15-oxo-2,5,8,11-tetraoxa-14-azanonadecan-19-oate (106)

To a solution of amine 104 (0.23 g; 0.57 mmol) in DMF (10 mL) were added carboxylic acid 105 (0.29 g; 0.72 mmol), HATU (0.27 g; 0.69 mmol), and DIPEA (0.50 mL, 2.9 mmol) at room temperature. Reaction mixture was allowed to stir for 2 h, then poured into 0.05% aqueous TFA (15 mL) and extracted with EtOAc (2×25 mL). Organic layer was washed with water and brine and dried over sodium sulfate. Solvents were removed under vacuum to afford crude compound 106 as an oil (0.50 g), which was use further without purification. LRMS (ESI): m/z 774.9 [M+H]⁺, Calcd for C₄₀H₅₉N₃O₁₂ m/z 774.4.

### Preparation of (S)-16-(1-(9H-fluoren-9-yl)-3-oxo-2,7,10-trioxa-4-azatridecan-13-amido)-15-oxo-2,5,8,11-tetraoxa-14-azanonadecan-19-oic acid (107)

To a solution of crude ester 106 (0.25 g, 0.32 mmol) in DCM (10 mL) were added TFA (4.0 mL), and the resulting solution was allowed to stir at room temperature for 6 h. Solvents were removed in vacuum to give 0.23 g (0.32 mmol, quant. yield) of crude compound 107 as an oil. LRMS (ESI): m/z 718.4 [M+H]⁺, Calcd for C₃₆H₅₁N₃O₁₂ m/z 718.4.

### Preparation of perfluorophenyl (S)-16-(1-(9H-fluoren-9-yl)-3-oxo-2,7,10-trioxa-4-azatridecan-13-amido)-15-oxo-2,5,8,11-tetraoxa-14-azanonadecan-19-oate (108)

To a solution of crude acid 107 (0.23 g; 0.32 mmol) in anhydrous THF (10 mL) were added DCC (0.33 g; 1.57 mmol) and pentafluoro phenol (0.29 g; 1.57 mmol) at room temperature. Reaction mixture was allowed to stir overnight at room temperature, then filtered, and concentrated under vacuum. The residue was purified by silica gel chromatography using 0-10% MeOH in DCM gradient to give 0.23 g of PFP-ester 106 a colorless oil (0.23 g, 0.26 mmol, 81% yield). LRMS (ESI): m/z 884.9 [M+H]⁺, Calcd for C₄₂H₅₀F₅N₃O₁₂ m/z 884.3.

### Preparation of (2S,3S,4S,5R,6S)-6-(2-((16R,21S,24S)-16-(3-(2-(2-aminoethoxy)ethoxy)propanamido)-21-isopropyl-24-methyl-15,19,22-trioxo-2,5,8,11-tetraoxa-14,20,23-triazapentacosan-25-amido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (109)

To a solution of amine 17 (10 mg; 10 μmol) in anhydrous DMF (2 mL) were added PFP-ester 106 (11.5 mg; 13 μmol), followed by HOAt (3 mg, 22 μmol) and DIPEA (10 μL) at room temperature. Reaction mixture was allowed to stir for 1 h, then piperidine (50 μL) was added to directly to the mixture and stirring continued for 30 mins. Reaction mixture was quenched by adding 2 mL of aqueous 0.05% TFA solution and purified by reversed-phase prep HPLC (C18, 0-50% v/v MeCN—H₂O with 0.05% TFA). Pure fractions were lyophilized to give 13 mg of compound 109 (9 μmol, 90% yield) as a pale-yellow solid. LRMS (ESI): m/z 1422.6 [M+H]⁺, Calcd for C₆₈H₉₅N₉O₂₄ m/z 1422.7.

### Preparation of (2S,3S,4S,5R,6S)-6-(2-((16S,21S,24S)-16-(3-(2-(2-(3-(5-((R)-16-(3-(((S)-1-(((S)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1-oxopropan-2-yl)amino)-3-methyl-1-oxobutan-2-yl)amino)-3-oxopropyl)-15,18,28-trioxo-2,5,8,11,21,24-hexaoxa-14,17,27-triazahentriacontan-31-amido)-2-((1,2-dimethylhydrazinyl)methyl)-1H-indol-1-yl)propanamido)ethoxy)ethoxy)propanamido)-21-isopropyl-24-methyl-15,19,22-trioxo-2,5,8,11-tetraoxa-14,20,23-triazapentacosan-25-amido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (110)

To a solution of amine 110 (13 mg, 9 μmol) in 2.5 mL of anhydrous DMF were added bis-PFP-ester 40 (4.2 mg, 4.5 μmol), followed by HOAt (2.4 mg, 18 μmol) and DIPEA (5 L). Reaction mixture was allowed to stir for 30 mins, then piperidine (50 μL) was added directly to the mixture and stirring continued for 30 min. Reaction mixture was purified by reversed-phase prep HPLC (C18, 0-50% v/v MeCN—H₂O with 0.05% TFA). Lyophilization of pure fractions gave 5 mg of compound 110 (1.6 μmol, 36% yield) as a pale-yellow solid. LRMS (ESI): m/z 1593.3 [M+2H]²⁺, Calcd for C₁₅₄H₂₁₀N₂₂O₅₁ m/z 1593.2.

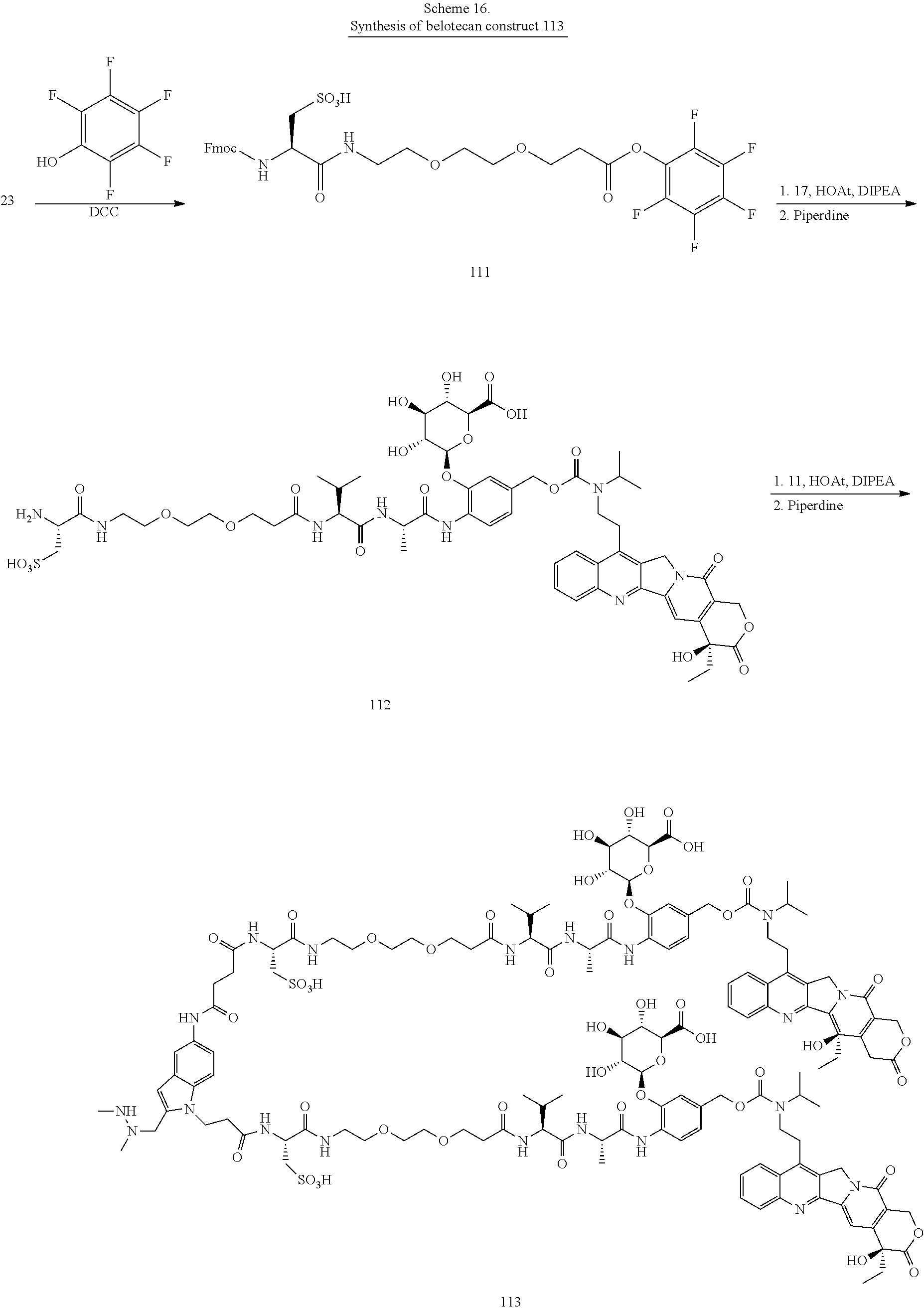

### Preparation of (R)-2-((((9H-fluoren-9-yl)methoxy)carbonyl)amino)-3-oxo-3-((2-(2-(3-oxo-3-(perfluorophenoxy)propoxy)ethoxy)ethyl)amino)propane-1-sulfonic acid (111)

To a mixture of compound 23 (136 mg, 0.25 mmol) and pentafluorophenol (136 mg, 0.75 mmol) in 2 mL of anhydrous THF were added DCC (155 mg, 0.75 mmol) at room temperature. Reaction mixture was stirred overnight, solids were filtered off, solvent was removed under reduced pressure, and the residue was purified by silica gel chromatography (EtOAc-hexane, 0-50% v/v gradient) to obtain PFP-ester 111 (27 mg, 38 μmol, 15% yield) as a colorless oil. LRMS (ESI): m/z 717.2 [M+H]⁺, Calcd for C₃₁H₂₉F₅N₂O₁₀S m/z 717.2.

### Preparation of (2S,3S,4S,5R,6S)-6-(2-((2S,5S,18R)-18-amino-5-isopropyl-2-methyl-4,7,17-trioxo-19-sulfo-10,13-dioxa-3, 6,16-triazanonadecanamido)-5-((((2-((S)-4-ethyl-4-hydroxy-3,14-dioxo-3, 4,12,14-tetrahydro-1H-pyrano[3, 4′:6, 7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (112)

To a stirred solution of compound 17 (25 mg, 26 μmol) in anhydrous DMF (1.0 mL) were added DIPEA (14 μL, 73 μmol) and HOAt (5 mg, 35 μmol), followed by PFP-ester 111 (19 mg, 27 μmol) in one portion at room temperature. Reaction mixture was stirred for 1 h, then purified by reversed-phase chromatography (C18, 0-100% v/v MeCN—H₂O with 0.05% TFA). Lyophilized pure fractions gave 9 mg of compound 112 (7 μmol, 26% yield) as a yellow powder. LRMS (ESI): m/z 1255.4 [M+H]⁺, Calcd for C₅₇H₇₄N₈O₂₂S m/z 1255.5.

### Preparation of (2S,3S,4S,5R,6S)-6-(2-((2S,5S,18R)-23-((1-((2S,5S,18R)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-5-isopropyl-2-methyl-1,4,7,17,20-pentaoxo-18-(sulfomethyl)-10,13-dioxa-3,6,16,19-tetraazadocosan-22-yl)-2-((1,2-dimethylhydrazinyl)methyl)-1H-indol-5-yl)amino)-5-isopropyl-2-methyl-4,7,17,20,23-pentaoxo-18-(sulfomethyl)-10,13-dioxa-3,6,16,19-tetraazatricosanamido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (113)

To a solution of compound 112 (9 mg, 7 μmol) in anhydrous DMF (1.0 mL) were added DIPEA (2 μL, 10 μmol) and HOAt (1.4 mg, 10 μmol), followed by bis-PFP ester 11 (3.0 mg, 3.5 μmol) in one portion at room temperature. Reaction mixture was stirred for 30 min, then treated with piperidine (7 μL, 70 μmol), let stir for 15 minutes, and then directly purified by reversed-phase prep HPLC (C18, 0-70% v/v MeCN—H₂O with 0.05% TFA). Lyophilized pure fractions gave 6 mg of compound 113 (2.0 μmol, 57% yield) as a yellow powder. LRMS (ESI): m/z 1426.1 [M+2H]²⁺, Calcd for C₁₃₂H₁₆₈N₂₀O₄₇S₂ m/z 1426.0.

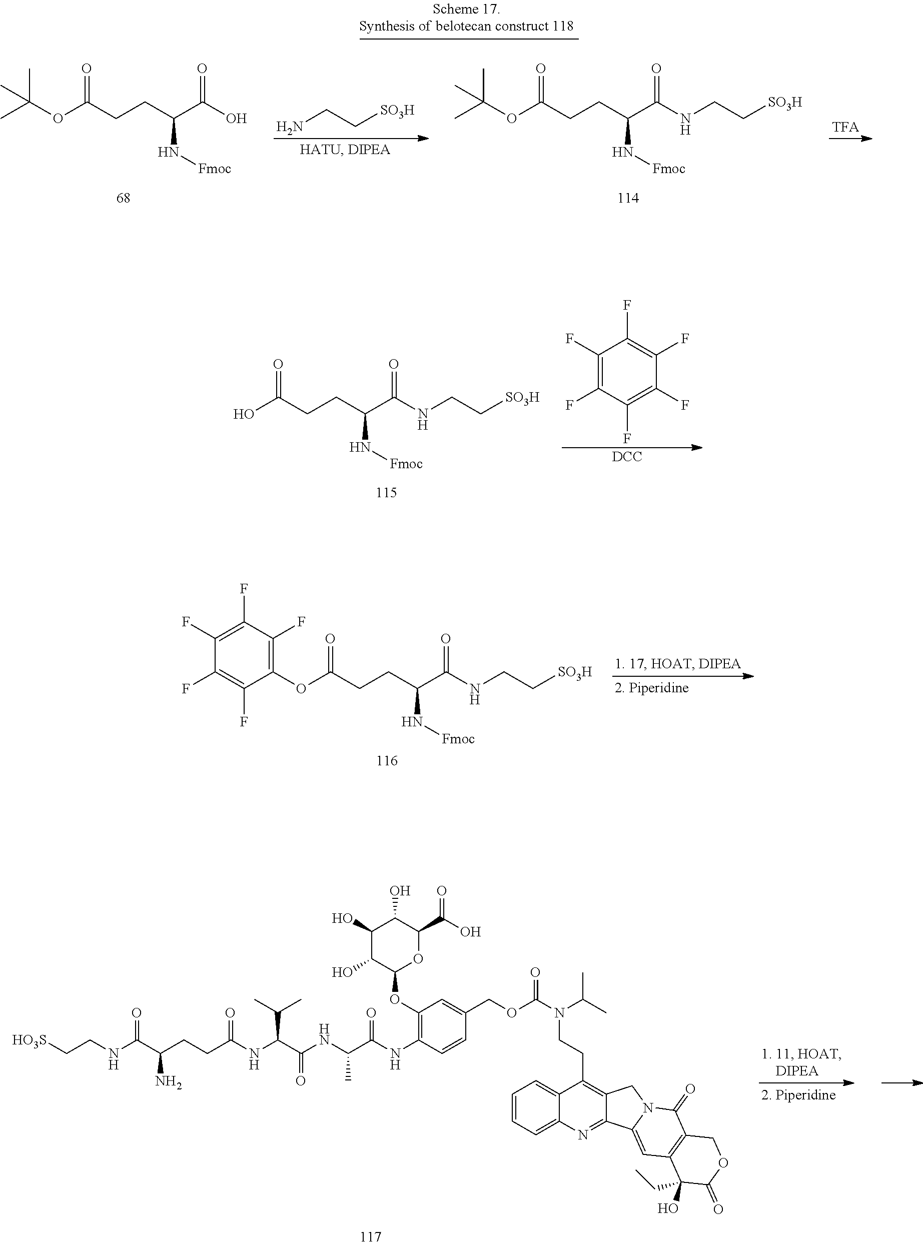

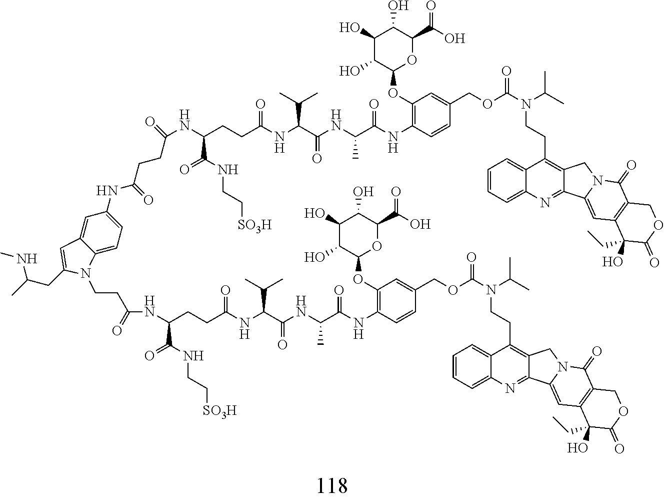 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CC[C@H](NC(=O)CCC(=O)Nc2ccc3c(c2)cc(CC(C)NC)n3CCC(=O)N[C@@H](CCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(=O)NCCS(=O)(=O)O)C(=O)NCCS(=O)(=O)O)C(C)C)c(O[C@@H]2O[C@H](C(=O)O)[C@@H](O)[C@H](O)[C@H]2O)c1)C(C)C]

### Preparation of (S)-2-(2-((((9H-fluoren-9-yl)methoxy)carbonyl)amino)-5-(tert-butoxy)-5-oxopentanamido)ethane-1-sulfonic acid (114)

To a 100 mL round bottom flask were added Fmoc-Glu(OtBu)-OH 68 (0.750 g, 1.77 mmol) and anhydrous DMF (20 mL), followed by HATU (1.02 g, 2.64 mmol), HOAt (0.250 g, 2.12 mmol), and DIPEA (500 μL) at room temperature. The resulting mixture was stirred for 45 min, then taurine (0.445 g, 3.53 mmol) was added, and the mixture was allowed to stir overnight. Reaction mixture was poured into water and extracted with DCM. Organic layer was washed with water, brine, dried over Na₂SO₄. Solvents were removed in vacuum to give crude compound 114 (1.4 g) as a white solid. LRMS (ESI-): m/z 531.2 [M−H]⁻, Calcd for C₂₆H₃₂N₂O₈S m/z 531.2.

### Preparation of (S)-4-((((9H-fluoren-9-yl)methoxy)carbonyl)amino)-5-oxo-5-((2-sulfoethyl)amino)pentanoic acid (115)

To a solution of crude compound 114 (1.4 g) in DCM (10 mL) were added TFA (5 mL) at room temperature. The reaction mixture was allowed to stir overnight, then solvents were removed in vacuum and the residue was purified by reversed-phase chromatography (C₁₈ column, 0-50% v/v MeCN—H₂O with 0.05% TFA) to give 0.74 g of product 115 as a white solid (1.6 mmol, 88% yield over 2 steps). LRMS (ESI−): m/z 475.1 [M−H]⁻, Calcd for C₂₂H₂₄N₂O₈S m/z 475.1.

### Preparation of (S)-2-(2-((((9H-fluoren-9-yl)methoxy)carbonyl)amino)-5-oxo-5-(perfluorophenoxy)pentanamido)ethane-1-sulfonic acid (116)

To a 100 mL round bottom flask with anhydrous THF (25 mL) were added carboxylic acid 115 (0.25 g, 0.53 mmol) and pentafluorophenol (0.49 g, 2.6 mmol), followed by DCC (0.83 g, 3.9 mmol) at room temperature. The resulting mixture was allowed to stir overnight at room temperature, then filtered, concentrated under vacuum, and purified by silica gel chromatography (0-10% MeOH in DCM gradient) to yield 0.18 g of PFP-ester 116 as a white solid (0.28 mmol, 53% yield). LRMS (ESI-): m/z 641.1 [M−H]⁻, Calcd for C₂₈H₂₃F₅N₂O₈S m/z 641.1.

### Preparation of (2S,3S,4S,5R,6S)-6-(2-((S)-2-((S)-2-((R)-4-amino-5-oxo-5-((2-sulfoethyl)amino)pentanamido)-3-methylbutanamido)propanamido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (117)

To a solution of PFP-ester 116 (30 mg, 48 μmol) in anhydrous DMF (3 mL) were added amine 17 (29 mg, 32 μmol), followed by HOAt (7.4 mg, 54 μmol) and DIPEA (30 μL) at room temperature. The resulting mixture was stirred for 45 min, then piperidine (50 μL) was added to the mixture and stirring continued for 30 min. Reaction mixture was purified directly by reversed-phase prep HPLC (C18 column, 0-50% v/v MeCN—H₂O with 0.05% TFA). Fractions containing product were concentrated and lyophilized to give 28 mg (24 μmol, 75% yield) of compound 117 as a bright yellow solid. LRMS (ESI−): m/z 1179.4 [M−H]⁻, Calcd for C₅₄H₆₈N₈O₂₀S m/z 1179.4.

### Preparation of (2S,3S,4S,5R,6S)-6-(2-((S)-2-((S)-2-((S)-4-(4-((1-(3-(((S)-5-(((S)-1-(((S)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1-oxopropan-2-yl)amino)-3-methyl-1-oxobutan-2-yl)amino)-1,5-dioxo-1-((2-sulfoethyl)amino)pentan-2-yl)amino)-3-oxopropyl)-2-((1,2-dimethylhydrazinyl)methyl)-1H-indol-5-yl)amino)-4-oxobutanamido)-5-oxo-5-((2-sulfoethyl)amino)pentanamido)-3-methylbutanamido)propanamido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (118)

To a solution of compound 117 (28 mg, 24 μmol) in 2 mL of anhydrous DMF were added bis-PFP-ester 11 (11 mg, 11.6 μmol), followed by HOAt (39 mg, 28 μmol) and DIPEA (21 μL). The resulting mixture was stirred for 45 min, then treated with piperidine (50 μL), stirred for additional 30 min, and purified by reversed-phase prep HPLC (C18 column, 0-50% v/v MeCN—H₂O with 0.05% TFA). Fractions containing the desired product were collected and lyophilized to give 15 mg of compound 118 as a pale-yellow solid (5.5 μmol, 47% yield). LRMS (ESI−): m/z 1350.0 [M−2H]²⁻, Calcd for C₁₂₆H₁₅₆N₂₀O₄₃S₂ m/z 1350.0.

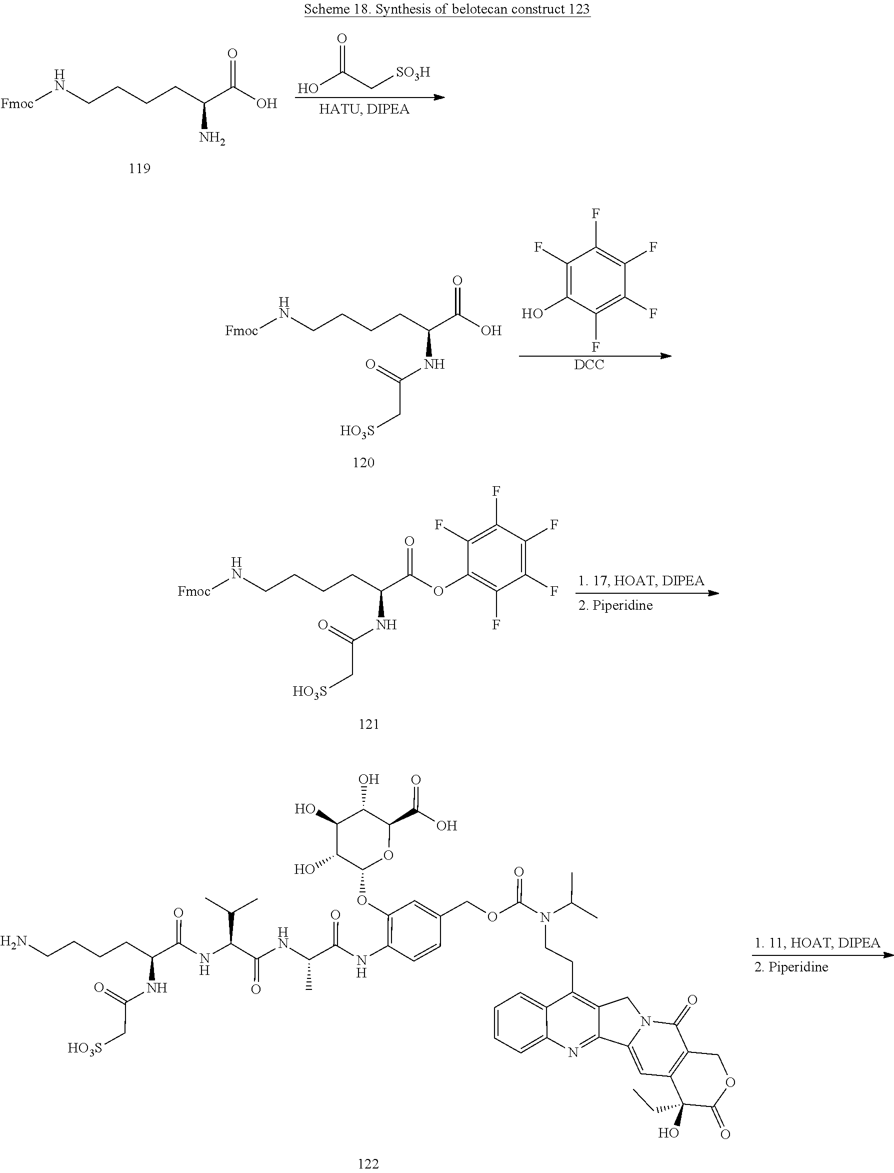

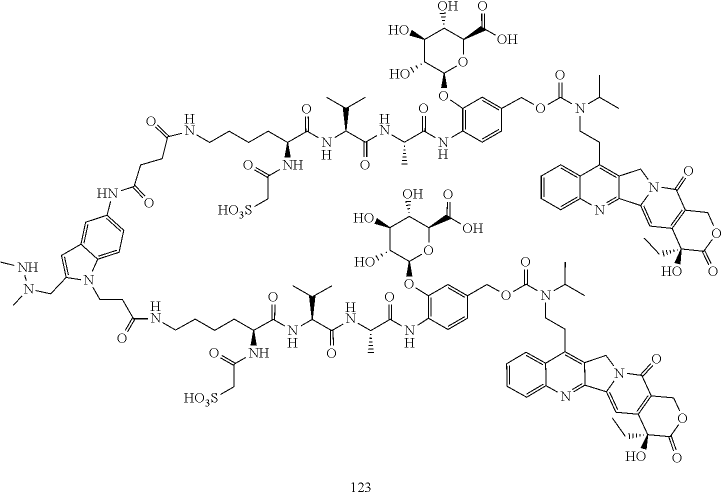 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CCCCNC(=O)CCC(=O)Nc2ccc3c(c2)cc(CN(C)NC)n3CCC(=O)NCCCC[C@H](NC(=O)CS(=O)(=O)O)C(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)NC(=O)CS(=O)(=O)O)C(C)C)c(O[C@@H]2O[C@H](C(=O)O)[C@@H](O)[C@H](O)[C@H]2O)c1)C(C)C]

### Preparation of N⁶-(((9H-fluoren-9-yl)methoxy)carbonyl)-N²-(2-sulfoacetyl)-L-lysine (120)

To a solution of 2-sulfoacetic acid (280 mg, 2.0 mmol) in DMF (3 mL) were added HATU (760 mg, 2.0 mmol) and DIPEA (695 μL, 4.0 mmol) at room temperature. After stirring this mixture for 30 minutes, amino acid 119 (330 mg, 0.90 mmol) was added, and stirring continued for one hour. Reaction mixture was directly purified by reversed phase HPLC using C18 column (H₂O/CH₃CN with 0.05% TFA, 90:10 to 0:100 v/v). Fractions containing the desired compound were pooled and lyophilized to yield compound 120 (280 mg, 0.57 mmol, 63% yield). LRMS (ESI): m/z 491.2 [M+H]⁺, Calcd for C₂₃H₂₆N₂O₈S m/z 491.1.

### Preparation of (S)-2-((6-((((9H-fluoren-9-yl)methoxy)carbonyl)amino)-1-oxo-1-(perfluorophenoxy)hexan-2-yl)amino)-2-oxoethane-1-sulfonic acid (121)

To a stirred mixture of carboxylic acid 120 (280 mg, 0.76 mmol) and pentafluorophenol (315 mg, 1.7 mmol) in DCM (5 mL) was added DCC (35 mg, 1.7 mmol) at room temperature. After stirring for one hour, reaction mixture was filtered, concentrated, and purified by reversed phased chromatography on C18 column (H₂O/CH₃CN with 0.05% TFA, 90:10 to 0:100 v/v) to afford compound 121 as a white solid (80 mg, 0.12 mmol, 16% yield). LRMS (ESI): m/z 657.1 [M+H]⁺, Calcd for C₂₉H₂₅F₅N₂O₈S m/z 657.1.

### Preparation of (2S,3S,4S,5R,6S)-6-(2-((S)-2-((S)-2-((S)-6-amino-2-(2-sulfoacetamido)hexanamido)-3-methylbutanamido)propanamido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (122)

To a mixture of amine 17 (10 mg, 11 μmol) and PFP-ester 121 (15 mg, 23 μmol) in anhydrous DMF (0.5 mL) were added DIPEA (4.4 μL, 26 μmol) at room temperature. After stirring overnight, piperidine (50 μL) was added to the reaction mixture. After stirring for 15 minutes at room temperature, reaction mixture was directly purified by reversed phase prep HPLC using C18 column (H₂O/CH₃CN with 0.05% TFA, 90:10 to 45:55 v/v). Fractions containing the desired compound were pooled and lyophilized to yield compound 122 (7 mg, 5.9 μmol, 54% yield). LRMS (ESI): m/z 1195.5 [M+H]⁺, Calcd for C₅₅H₇₀N₈O₂₀S m/z 1195.4.

### Preparation of (2S,3S,4S,5R,6S)-6-(2-((S)-2-((S)-2-((S)-6-(4-((1-(3-(((S)-6-(((S)-1-(((S)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1-oxopropan-2-yl)amino)-3-methyl-1-oxobutan-2-yl)amino)-6-oxo-5-(2-sulfoacetamido)hexyl)amino)-3-oxopropyl)-2-((1,2-dimethylhydrazineyl)methyl)-1H-indol-5-yl)amino)-4-oxobutanamido)-2-(2-sulfoacetamido)hexanamido)-3-methylbutanamido)propanamido)-5-((((2-((S)-4-ethyl-4-hydroxy-3,14-dioxo-3,4,12,14-tetrahydro-1H-pyrano[3′,4′:6,7]indolizino[1,2-b]quinolin-1 1-yl)ethyl)(isopropyl)carbamoyl)oxy)methyl)phenoxy)-3,4,5-trihydroxytetrahydro-2H-pyran-2-carboxylic acid (123)

To a stirred mixture of amine 122 (7 mg, 6 μmol) and bis-PFP-ester 11 (2.6 mg, 2.8 μmol) in DMF (0.5 mL) were added DIPEA (5 μL, 26 μmol) at room temperature. The resulting mixture was stirred for 2 hours, then piperidine (50 μL) was added to the mixture. After stirring for 15 minutes at room temperature, the reaction mixture was directly purified by reversed phase prep HPLC using C18 column (H₂O/CH₃CN with 0.05% TFA, 90:10 to 45:55 v/v). Fractions containing the desired compound were pooled and lyophilized to yield compound 123 (3 mg, 1 μmol, 36% yield). LRMS (ESI): m/z 1365.5 [M+H]²⁺, Calcd for C₁₂₈H₁₆₀N₂₀O₄₃S₂ m/z 1365.5.

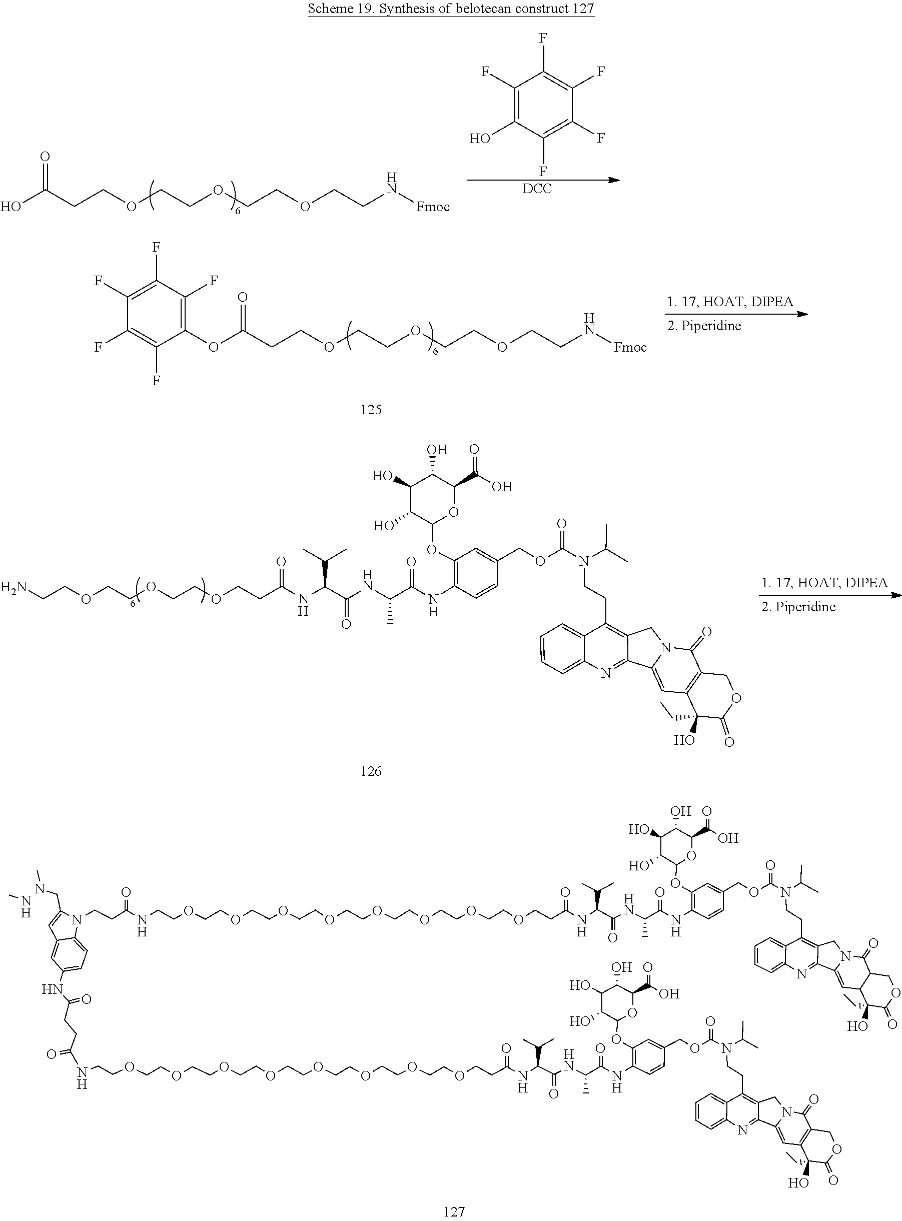

### Preparation of perfluorophenyl 1-(9H-fluoren-9-yl)-3-oxo-2,7,10,13,16,19,22,25,28-nonaoxa-4-azahentriacontan-31-oate (125)

To a stirred mixture of carboxylic acid 124 (100 mg, 0.15 mmol) and pentafluorophenol (140 mg, 0.75 mmol) in anhydrous THF (2 mL) were added DCC (37 mg, 0.18 mmol) in one portion at room temperature. The resulting mixture was stirred overnight, filtered, and concentrated under vacuum. The residue was purified by reversed-phase chromatography (C18 column, 0-70% v/v MeCN—H₂O with 0.05% TFA) to afford 120 mg of compound 125 (0.14 mmol, 93% yield) as a clear colorless oil. LRMS (ESI): m/z 830.3 [M+H]⁺, Calcd for C₄₀H₄₈F₅NO₁₂ m/z 830.3.

### Preparation of (2S,3S,4S,5R,6S)-6-(2-((29S,32S)-1-amino-29-isopropyl-32-methyl-27,30-dioxo-3,6,9,12,15,18,21,24-octaoxa-28,31-diazatritriacontan-33-amido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (126)

A solution of amine 17 (55 mg, 58 μmol) in 2 mL of anhydrous DMF was treated with DIPEA (20 μL, 0.12 mmol) and HOAt (8 mg, 58 μmol), and then combined with PFP-ester 125 (48 mg, 58 μmol) in DMF (1 mL) at room temperature. The resulting mixture was stirred for 30 minutes, then piperidine (115 μL, 115 μmol) was added to the mixture. After 20 minutes, reaction mixture was purified by reversed-phase prep HPLC (C18 column, 0-50% v/v MeCN—H₂O with 0.05% TFA). Pure fractions containing product were combined and lyophilized to give 49 mg of compound 126 as a yellowish solid (36 μmol, 62% yield). LRMS (ESI): m/z 1368.6 [M+H]⁺, Calcd for C₆₆H₉₃N₇O₂₄ m/z 1368.6.

### Preparation of (2S,3S,4S,5R,6S)-6-(2-((2S,5S)-38-((1-((2S,5S)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-5-isopropyl-2-methyl-1,4,7,35-tetraoxo-10,13,16,19,22,25,28,31-octaoxa-3,6,34-triazaheptatriacontan-37-yl)-2-((1,2-dimethylhydrazineyl)methyl)-1H-indol-5-yl)amino)-5-isopropyl-2-methyl-4,7,35,38-tetraoxo-10,13,16,19,22,25,28,31-octaoxa-3,6,34-triazaoctatriacontanamido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (127)

To a mixture of compound 126 (49 mg, 36 μmol) and DIPEA (13 μL, 72 μmol) in 2 mL of DMA were added bis-PFP-ester 11 (14.6 mg, 16 μmol) in one portion at room temperature, followed by HOAt (5 mg, 36 μmol). The resulting mixture was stirred at room temperature for 30 minutes, then piperidine (21 μL), was added, and stirring continued for 20 minutes. Reaction mixture was directly purified by reversed-phase prep HPLC (C18 column, 0-50% v/v MeCN—H₂O with 0.05% TFA). Lyophilized fractions gave 32 mg of compound 127 (10 μmol, 63% yield) as a yellow powder. LRMS (ESI): m/z 1539.3 [M+H]²⁺, Calcd for C₁₅₀H₂₀₆N₁₈O₅ m/z 1538.7.

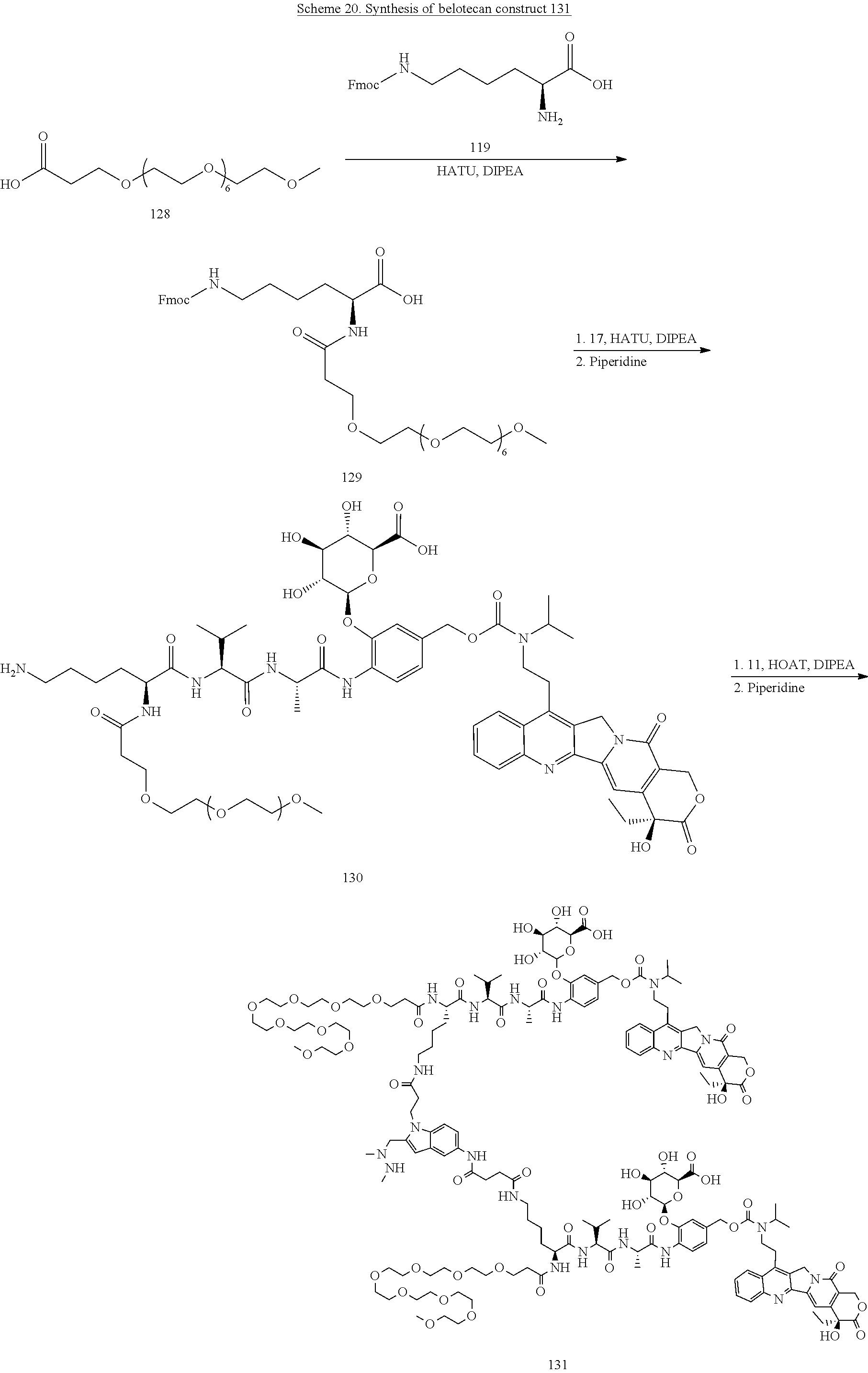

### Preparation of N⁶-(((9H-fluoren-9-yl)methoxy)carbonyl)-N²-(3-(2-(2-methoxyethoxy)ethoxy)propanoyl)-L-lysine (129)

To a solution of mPEG8-acid 128 (100 mg, 0.24 mmol) in 2 mL of anhydrous DMF were added DIPEA (0.13 mL, 0.72 mmol) and HATU (93 mg, 0.24 mmol) at room temperature. The resulting mixture was stirred for one hour, then Lys(Fmoc)-OH 119 (89 mg, 0.24 mmol) was added to the mixture, and stirring continued for one hour. Reaction mixture was directly purified by reversed-phase chromatography HPLC (C18, 0-70% v/v MeCN—H₂O with 0.05% TFA) to give 120 mg of compound 129 (0.16 mmol, 67% yield) as a colorless oil. LRMS (ESI): m/z 763.4 [M+H]⁺, Calcd for C₃₉H₅₈N₂O₁₃ m/z 763.4.

### Preparation of (2S,3S,4S,5R,6S)-6-(2-((28S,31S,34S)-28-(4-aminobutyl)-31-isopropyl-34-methyl-26,29,32-trioxo-2,5,8,11,14,17,20,23-octaoxa-27,30,33-triazapentatriacontan-35-amido)-5-((((2-((S)-4-ethyl-4-hydroxy-3,14-dioxo-3, 4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (130)

To a solution of carboxylic acid 129 (45 mg, 59 μmol) in 3 mL of anhydrous DMF were added DIPEA (21 μL, 120 μmol) and HATU (22 mg, 59 μmol) at room temperature. The resulting mixture was stirred for 20 minutes and combined with amine 17 (55 mg, 58 μmol) in 1 mL of DMF. Reaction mixture was stirred for 30 minutes, then piperidine (115 μL, 1.2 mmol) was added to the mixture at room temperature. After 20 minutes, reaction mixture was directly purified by reversed phase prep HPLC (C18, 0-50% v/v MeCN—H₂O with 0.05% TFA). Lyophilization of pure fractions afforded 34 mg (23 μmol, 40% yield) of compound 130 as a yellow powder. LRMS (ESI): m/z 1467.7 [M+H]⁺, Calcd for C₇₁H₁₀₂N₈O₂₅ m/z 1467.7.

### Preparation of (2S,3S,4S,5R,6S)-6-(2-((28S,31S,34S)-28-(4-(3-(5-((S)-28-(((S)-1-(((S)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1-oxopropan-2-yl)amino)-3-methyl-1-oxobutan-2-yl)carbamoyl)-26,34-dioxo-2,5,8,11,14,17,20,23-octaoxa-27,33-diazaheptatriacontan-37-amido)-2-((1,2-dimethylhydrazineyl)methyl)-1H-indol-1-yl)propanamido)butyl)-31-isopropyl-34-methyl-26,29,32-trioxo-2,5,8,11,14,17,20,23-octaoxa-27,30,33-triazapentatriacontan-35-amido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (131)

To a mixture of compound 130 (34 mg, 23 μmol) and DIPEA (8 μL, 46 μmol) in 2 mL of DMA were added bis-PFP ester 11 (9.4 mg, 10.5 μmol), followed by HOAt (3 mg, 23 μmol) at room temperature. The resulting mixture was allowed to stand for 30 minutes at room temperature, then piperidine (21 μL, 0.21 mmol) was added to the mixture at room temperature. After 20 minutes, reaction mixture was directly purified by reversed phase prep HPLC (C18, 0-50% v/v MeCN—H₂O with 0.05% TFA). Pure fractions were combined and lyophilized to afford compound 131 as a yellow solid (23 mg, 7 μmol, 67% yield). LRMS (ESI): m/z 1638.3 [M+H]²⁺, Calcd for C₁₆₀H₂₂₄N₂₀O₅₃ m/z 1638.8.

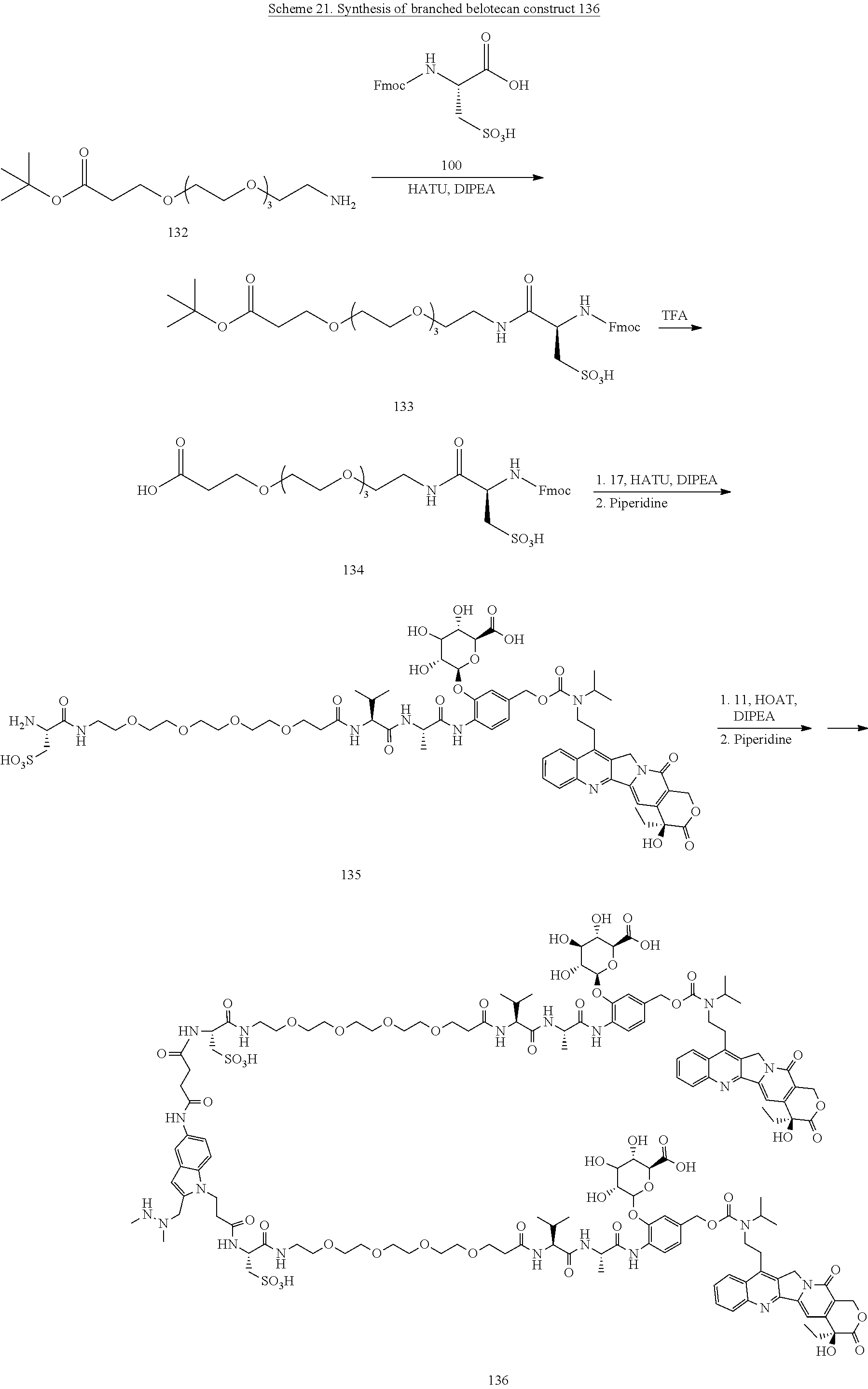

### Preparation of (R)-21-((((9H-fluoren-9-yl)methoxy)carbonyl)amino)-2,2-dimethyl-4,20-dioxo-3,7,10,13,16-pentaoxa-19-azadocosane-22-sulfonic acid (133)

To a mixture of Fmoc-L-cysteic acid 100 (391 mg, 1.0 mmol) and amine 132 (321 mg, 1.0 mmol) in anhydrous DMF (2 mL) were added HATU (400 mg, 1.05 mmol) and DIPEA (0.52 mL, 3 mmol). Reaction mixture was stirred for one hour, and then directly purified by reversed phase chromatography (C18, 0-50% v/v MeCN—H₂O with 0.05% TFA) to obtain compound 133 as a colorless oil (500 mg, 0.72 mmol, 72% yield). LRMS (ESI−): m/z 693.3 [M−H]⁻, Calcd for C₃₃H₄₆N₂O₁₂S m/z 693.3.

### Preparation of (R)-1-(9H-fluoren-9-yl)-3,6-dioxo-5-(sulfomethyl)-2,10,13,16,19-pentaoxa-4,7-diazadocosan-22-oic acid (134)

To a solution of compound 133 (100 mg, 0.14 mmol) in DCM (2 mL) were added TFA (2 mL) at ambient temperature. Reaction mixture was stirred for 10 minutes, then solvents were removed under vacuum, and the residue was purified by reversed phase chromatography (C18, 0-75% v/v MeCN—H₂O with 0.05% TFA) to give compound 134 as a colorless oil (80 mg, 0.12 mmol, 86% yield). LRMS (ESI−): m/z 637.2 [M−H]⁻, Calcd for C₂₉H₃₈N₂O₁₂S m/z 637.2.

### Preparation of (2S,3S,4S,5R,6S)-6-(2-((2S,5S,24R)-24-amino-5-isopropyl-2-methyl-4,7,23-trioxo-25-sulfo-10,13,16,19-tetraoxa-3,6,22-triazapentacosanamido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (135)

To a solution of compound 134 (9 mg, 14 μmol) in anhydrous DMF (1.0 mL) were added DIPEA (7.4 μL, 42 μmol) and HATU (5 mg, 13 μmol) at room temperature. The resulting mixture was stirred for 30 minutes, and then combined with compound 46 (14 mg, 15 μmol) at room temperature. After one hour, piperidine (30 μL) was added to the reaction mixture, and stirring continued for 20 minutes. Reaction mixture was purified by reversed-phase prep HPLC (C18, 0-70% v/v MeCN—H₂O with 0.05% TFA). Lyophilized pure fractions gave 13 mg of compound 135 (10 μmol, 68% yield) as a yellow powder. LRMS (ESI): m/z 1343.5 [M+H]⁺, Calcd for C₆₁H₈₂N₈O₂₄S m/z 1343.5.

### Preparation of (2S,3S,4S,5R,6S)-6-(2-((2S,5S,24R)-28-(5-((2S,5S,24R)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-5-isopropyl-2-methyl-1,4,7,23,26-pentaoxo-24-(sulfomethyl)-10,13,16,19-tetraoxa-3,6,22,25-tetraazanonacosan-29-amido)-2-((1,2-dimethylhydrazinyl)methyl)-1H-indol-1-yl)-5-isopropyl-2-methyl-4,7,23,26-tetraoxo-24-(sulfomethyl)-10,13,16,19-tetraoxa-3,6,22,25-tetraazaoctacosanamido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (136)

To a solution of compound 135 (13 mg, 10 μmol) in anhydrous DMF (0.5 mL) were added DIPEA (5 μL, 15 μmol) and HOAt (2 mg, 15 μmol), followed by bis-PFP ester 11 (4.3 mg, 5 μmol) at room temperature. After 30 minutes, reaction was judged complete by LCMS analysis, and piperidine (10 μL, 97 μmol) was added directly to the mixture in one shot at room temperature. After 15 minutes, reaction mixture was purified by reversed phase prep HPLC (C18, 0-70% v/v MeCN—H₂O with 0.05% TFA). Lyophilized pure fractions gave 7.4 mg of compound 136 (2.4 μmol, 57% yield) as a yellow powder. LRMS (ESI): m/z 1514.2.1 [M+2H]²⁺, Calcd for C₁₄₀H₁₈₄N₂₀O₅₁S₂ m/z 1514.1.

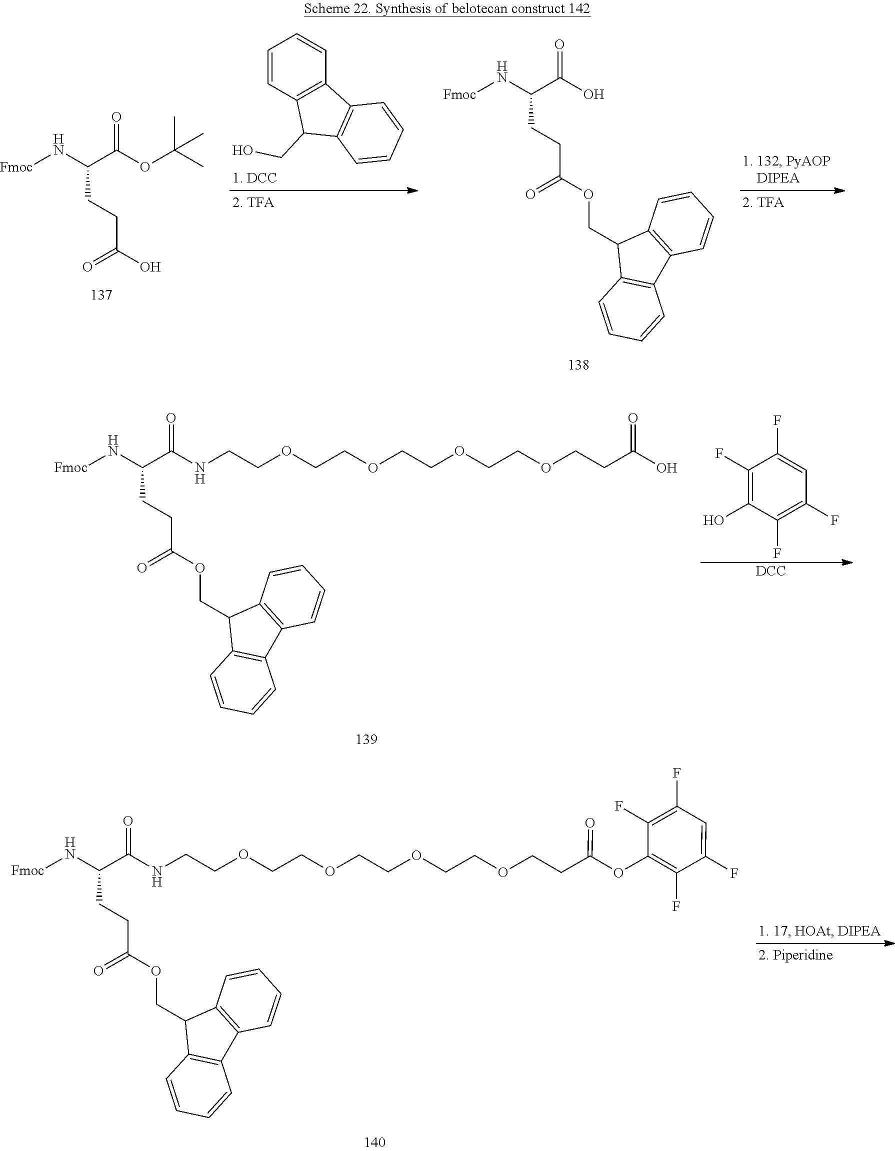

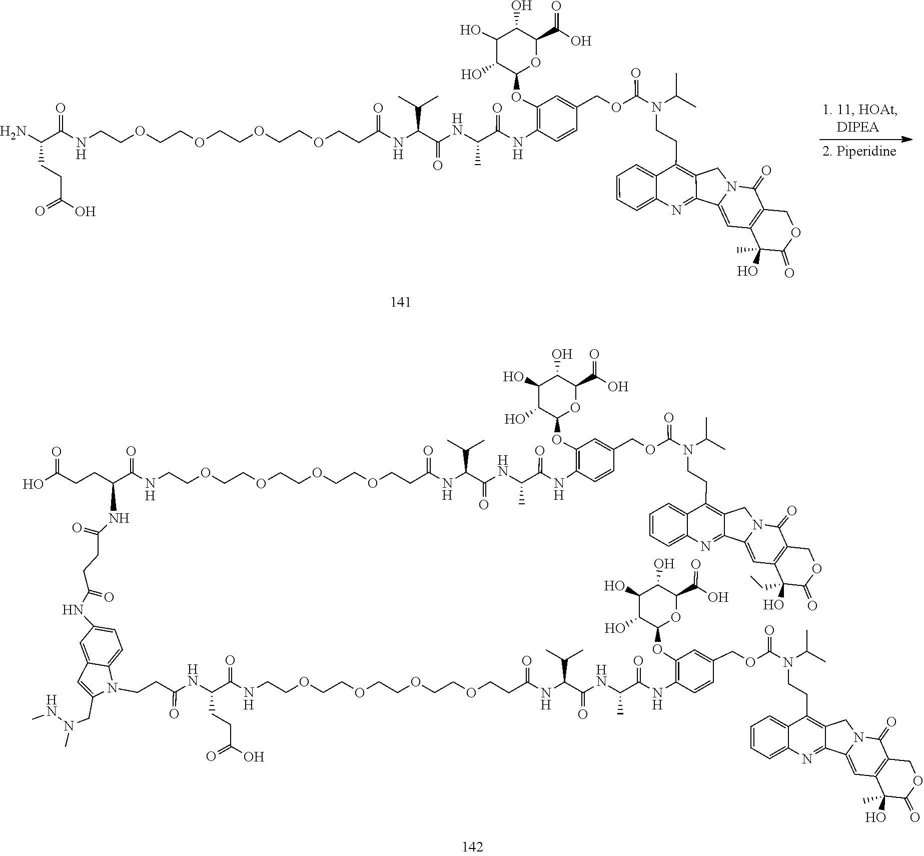

### Preparation of (S)-5-((9H-fluoren-9-yl)methoxy)-2-((((9H-fluoren-9-yl)methoxy)carbonyl)amino)-5-oxopentanoic acid (138)

To a mixture of Fmoc-Glu-OtBu 137 (426 mg, 1 mmol) and (9H-fluoren-9-yl)methanol (216 mg, 1.1 mmol) in 5 mL of anhydrous THF were added DCC (247 mg, 1.2 mmol) in one portion at room temperature. The resulting mixture was stirred overnight, filtered, and concentrated under vacuum. The residue was dissolved in DCM-TFA mixture (1:1 v/v, 6 mL) and let stand at room temperature for 30 minutes. Solvents were removed under vacuum, the residue was dissolved in 40 mL of EtOAc, washed with sat. ammonium chloride, water, and brine, dried over sodium sulfate, and purified by silica gel chromatography (0-25% v/v EtOAc-hexane) to afford 230 mg of Fmoc-Glu(OFm)-OH 138 as a colorless solid (0.42 mmol, 42% yield). LRMS (ESI): m/z 548.2 [M+H]⁺, Calcd for C₃₄H₂₉NO₆ m/z 548.2.

### Preparation of (S)-5-(3-((9H-fluoren-9-yl)methoxy)-3-oxopropyl)-1-(9H-fluoren-9-yl)-3,6-dioxo-2,10,13,16,19-pentaoxa-4,7-diazadocosan-22-oic acid (139)

To a mixture of Fmoc-Glu(OFm)-OH 138 (230 mg, 0.42 mmol) and amino-PEG4-OtBu 132 (162 mg, 0.46 mmol) in 2 mL of DMF were added DIPEA (0.22 mL, 1.26 mmol), followed by PyAOP (240 mg, 0.42 mmol) at room temperature. Reaction mixture was stirred for 30 minutes, then poured into sat. ammonium chloride solution and extracted with EtOAc. Organic layer was washed with brine, and dried over sodium sulfate. After removal of solvents in vacuum, the residue was reconstituted in DCM-TFA mixture (1:1 v/v, 4 mL) at room temperature and stirred for 15 minutes, then solvents were removed in vacuum and the residue was purified by reversed phase chromatography (C18, 0-70% v/v MeCN—H₂O with 0.05% TFA) to give 306 mg of compound 139 as a clear colorless oil (0.39 mmol, 92% yield). LRMS (ESI): m/z 795.3 [M+H]⁺, Calcd for C₄₅H₅₀N₂O₁₁ m/z 795.3.

### Preparation of 21-((9H-fluoren-9-yl)methyl) 1-(2,3,5,6-tetrafluorophenyl) (S)-18-((((9H-fluoren-9-yl)methoxy)carbonyl)amino)-17-oxo-4,7,10,13-tetraoxa-16-azahenicosanedioate (140)

To a mixture of compound 139 (145 mg, 0.18 mmol) and 2,3,5,6,-tatrafluorophenol (61 mg, 0.36 mmol) in 2 mL of THF were added DCC (45 mg, 0.36 mmol) in one portion at room temperature. The resulting mixture was stirred overnight, filtered, concentrated under vacuum and purified by reversed phase chromatography (C18, 0-80% v/v MeCN—H₂O with 0.05% TFA) to give 84 mg of TFP-ester 140 as a colorless oil (0.09 mmol, 50% yield). LRMS (ESI): m/z 965.3 [M+Na]⁺, Calcd for C₅₁H₅₀F₄N₂O₁₁ m/z 965.3.

### Preparation of (2S,5S,24S)-24-amino-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-1]-yl)ethyl)(isopropyl)carbamoyl)oxy)methyl)phenyl)amino)-5-isopropyl-2-methyl-1,4,7,23-tetraoxo-10,13,16,19-tetraoxa-3,6,22-triazaheptacosan-27-oic acid (141)

To a solution of compound 17 (19 mg, 20 μmol) in 3 mL of anhydrous DMF were added DIPEA (9 μL, 60 μmol) and HOAt (2.7 mg, 20 μmol), followed by TFP ester 140 (19 mg, mol) in one portion at room temperature. Reaction mixture was stirred for 30 minutes, monitored by LCMS analysis. After reaction was judged complete, piperidine (40 μL) was added to the mixture, and stirring continued for 20 minutes. Reaction mixture was then purified by reversed-phase prep HPLC (C18, 0-50% v/v MeCN—H₂O with 0.05% TFA). Pure fractions were lyophilized to afford 14.6 mg of compound 141 as a yellow powder (11 μmol, 55% yield). LRMS (ESI): m/z 1321.5 [M+H]⁺, Calcd for C₆₃H₈₄N₈O₂₃ m/z 1321.6.

### Preparation of (2S,3S,4S,5R,6S)-6-(2-((2S,5S,24S)-28-(5-((2S,5S,24S)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-24-(2-carboxyethyl)-5-isopropyl-2-methyl-1,4,7,23,26-pentaoxo-10,13,16,19-tetraoxa-3,6,22,25-tetraazanonacosan-29-amido)-2-((1,2-dimethylhydrazinyl)methyl)-1H-indol-1-yl)-24-(2-carboxyethyl)-5-isopropyl-2-methyl-4,7,23,26-tetraoxo-10,13,16,19-tetraoxa-3,6,22,25-tetraazaoctacosanamido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (142)

To a solution of compound 141 (14.6 mg, 11 μmol) in 2 mL of DMA were added DIPEA (6 μL, 33 μmol) and HOAt (1.5 mg, 11 μmol), followed by bis-PFP ester 11 (4.5 mg, 5 mol) in one portion at room temperature. The resulting mixture was allowed to stand at room temperature for 30 minutes, then piperidine (10 μL) was added directly to the mixture. After 20 minutes, reaction mixture was purified by reversed-phase prep HPLC (C18, 0-50% v/v MeCN—H₂O with 0.05% TFA). Pure fractions were combined and lyophilized to give 6 mg (2 μmol, 40% yield) of compound 142 as a yellow solid. LRMS (ESI): m/z 1491.2 [M+2H]²⁺, Calcd for C₁₄₄H₁₈₈N₂₀O₄₉ m/z 1491.6.

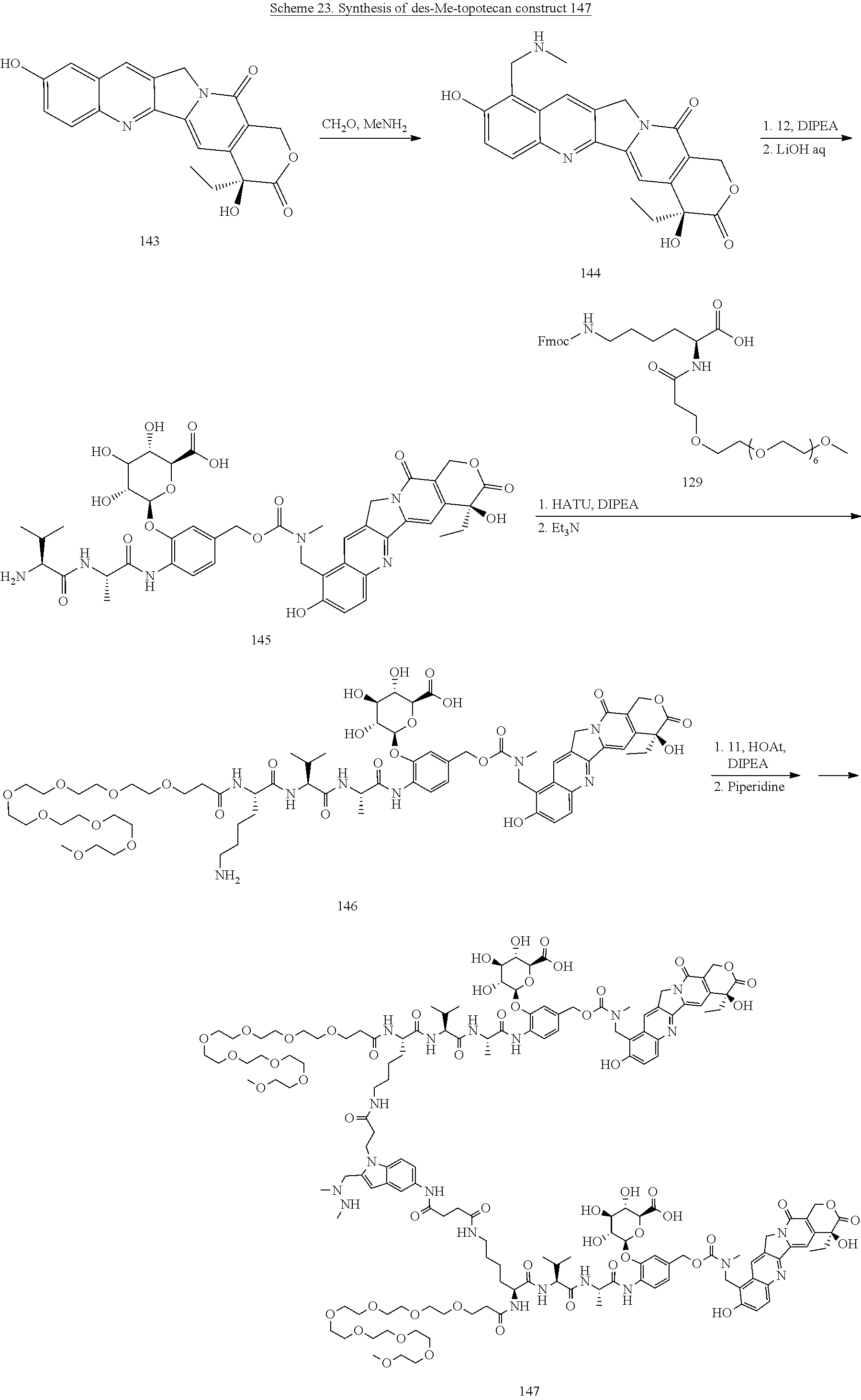

### Preparation of (S)-4-ethyl-4,9-dihydroxy-10-((methylamino)methyl)-1,12-dihydro-14H-pyrano[3,4′:6,7]indolizino[1,2-b]quinoline-3,14(4H)-dione (144)

To a solution of 10-hydroxycamtothecin 143 (500 mg, 1.37 mmol) in acetic acid (30 mL) and EtOH (15 mL) were added formaldehyde (1 mL, 37 wt % in H₂O) and MeNH₂ (1 mL, 40% w/w water solution). Reaction mixture was allowed to stir overnight at room temperature, then concentrated under reduced pressure. The residue was purified by reversed phase chromatography (C18, 0-70% v/v MeCN—H₂O with 0.05% TFA). Pure fractions were collected and lyophilized to obtain des-Me-topotecan 144 as a light-yellow solid (150 mg, 0.46 mmol, 27% yield). LRMS (ESI): m/z 408.2 [M+H]⁺, Calcd for C₂₂H₂₁N₃O₅ m/z 408.2.

### Preparation of (2S,3S,4S,5R,6S)-6-(2-((S)-2-((S)-2-amino-3-methylbutanamido)propanamido)-5-((((((S)-4-ethyl-4,9-dihydroxy-3,14-dioxo-3,4,12,14-tetrahydro-1H-pyrano[3′,4′:6,7]indolizino[1,2-b]quinolin-10-yl)methyl)(methyl)carbamoyl)oxy)methyl)phenoxy)-3,4,5-trihydroxytetrahydro-2H-pyran-2-carboxylic acid (145)

To a stirred solution of des-Me-topotecan 144 (25 mg, 61 μmol) in DMF (1.5 mL) were added HOAt (8.5 mg, 62 μmol) and DIPEA (30 μL, 184 μmol) at room temperature. The resulting mixture was then treated with PNP-carbonate 12 (58 mg, 62 μmol) in one portion at room temperature. Reaction mixture was stirred overnight until all the starting materials were consumed as judged by LCMS analysis. Reaction mixture was poured into 10 mL of water, and the resulting precipitate was collected and dissolved in THF (2 mL). The THF solution was then treated with aq. LiOH (1 mL, 1M) slowly at 0° C. and stirred for 30 min. Reaction mixture was allowed to slowly warm to room temperature and stirred for an additional hour, quenched by adding 1M aq. HCl to pH ˜4, filtered, and purified by reversed-phase prep HPLC (C18, 0-70% v/v MeCN—H₂O with 0.05% TFA). Pure fractions were collected and lyophilized to obtain compound 145 as a yellow solid (25 mg, 27 μmol, 44% yield). LRMS (ESI): m/z 919.3 [M+H]⁺, Calcd for C₄₄H₅₀N₆O₁₆ m/z 919.3.

### Preparation of (2S,3S,4S,5R,6S)-6-(2-((28S,31S,34S)-28-(4-aminobutyl)-31-isopropyl-34-methyl-26,29,32-trioxo-2,5,8,11,14,17,20,23-octaoxa-27,30,33-triazapentatriacontan-35-amido)-5-((((((S)-4-ethyl-4,9-dihydroxy-3,14-dioxo-3,4,12,14-tetrahydro-1H-pyrano[3′,4′:6,7]indolizino[1,2-b]quinolin-10-yl)methyl)(methyl)carbamoyl)oxy)methyl)phenoxy)-3,4,5-trihydroxytetrahydro-2H-pyran-2-carboxylic acid (146)

To a solution of compound 128 (21 mg, 27.5 μmol) in DMF (2 mL) were added HATU (10 mg, 31 μmol) and DIPEA (14 μL, 82 μmol) at room temperature. The resulting mixture was stirred for one hour, then compound 145 (25 mg, 27 μmol) was added to the mixture, and stirring continued for 1 h, until coupling was judged complete by LCMS analysis. Next, reaction mixture was treated with triethylamine (0.4 mL) and stirred at room temperature for 5 h. Reaction mixture was purified by reversed phase prep HPLC (C18, 0-70% v/v MeCN—H₂O with 0.05% TFA). Pure fractions were collected and lyophilized to obtain compound 146 as a yellow solid (26 mg, 18 μmol, 67% yield). LRMS (ESI): m/z 1441.6 [M+H]⁺, Calcd for C₆₈H₉₆N₈O₂₆ m/z 1441.6.

### Preparation of (2S,3S,4S,5R,6S)-6-(2-((28S,31S,34S)-28-(4-(3-(5-((S)-28-(((S)-1-(((S)-1-((2-(((2S,3R,4S,5S,6S)-6-carboxy-3,4,5-trihydroxytetrahydro-2H-pyran-2-yl)oxy)-4-((((((S)-4-ethyl-4,9-dihydroxy-3,14-dioxo-3,4,12,14-tetrahydro-1H-pyrano[3′,4′:6,7]indolizino[1,2-b]quinolin-10-yl)methyl)(methyl)carbamoyl)oxy)methyl)phenyl)amino)-1-oxopropan-2-yl)amino)-3-methyl-1-oxobutan-2-yl)carbamoyl)-26,34-dioxo-2,5,8,11,14,17,20,23-octaoxa-27,33-diazaheptatriacontan-37-amido)-2-((1,2-dimethylhydrazinyl)methyl)-1H-indol-1-yl)propanamido)butyl)-31-isopropyl-34-methyl-26,29,32-trioxo-2,5,8,11,14,17,20,23-octaoxa-27,30,33-triazapentatriacontan-35-amido)-5-((((((S)-4-ethyl-4,9-dihydroxy-3,14-dioxo-3,4,12,14-tetrahydro-1H-pyrano[3′,4′:6,7]indolizino[1,2-b]quinolin-10-yl)methyl)(methyl)carbamoyl)oxy)methyl)phenoxy)-3,4,5-trihydroxytetrahydro-2H-pyran-2-carboxylic acid (147)

To a solution of compound 146 (26 mg, 18 μmol) in DMF (1.5 mL) were added DIPEA (10 μL, 55 μmol) and HOAt (7 mg, 23 μmol), followed by bis-PFP ester 11 (8.4 mg, 9 mol) in one portion at room temperature. Reaction mixture was stirred for 30 minutes until coupling was judged complete by LCMS analysis, then diethylamine (37 μL, 0.36 mmol) was added to the mixture and stirring continued for 2 hours. Reaction mixture was purified by reversed phase prep HPLC (C18, 0-70% v/v MeCN—H₂O with 0.05% TFA). Pure fractions were collected and lyophilized to give compound 147 as a yellow solid (18 mg, 6 μmol, 67% yield). LRMS (ESI): m/z 1612.2 [M+2H]²⁺, Calcd for C₁₅₄H₂₁₂N₂₀O₅₅ m/z 1612.2.

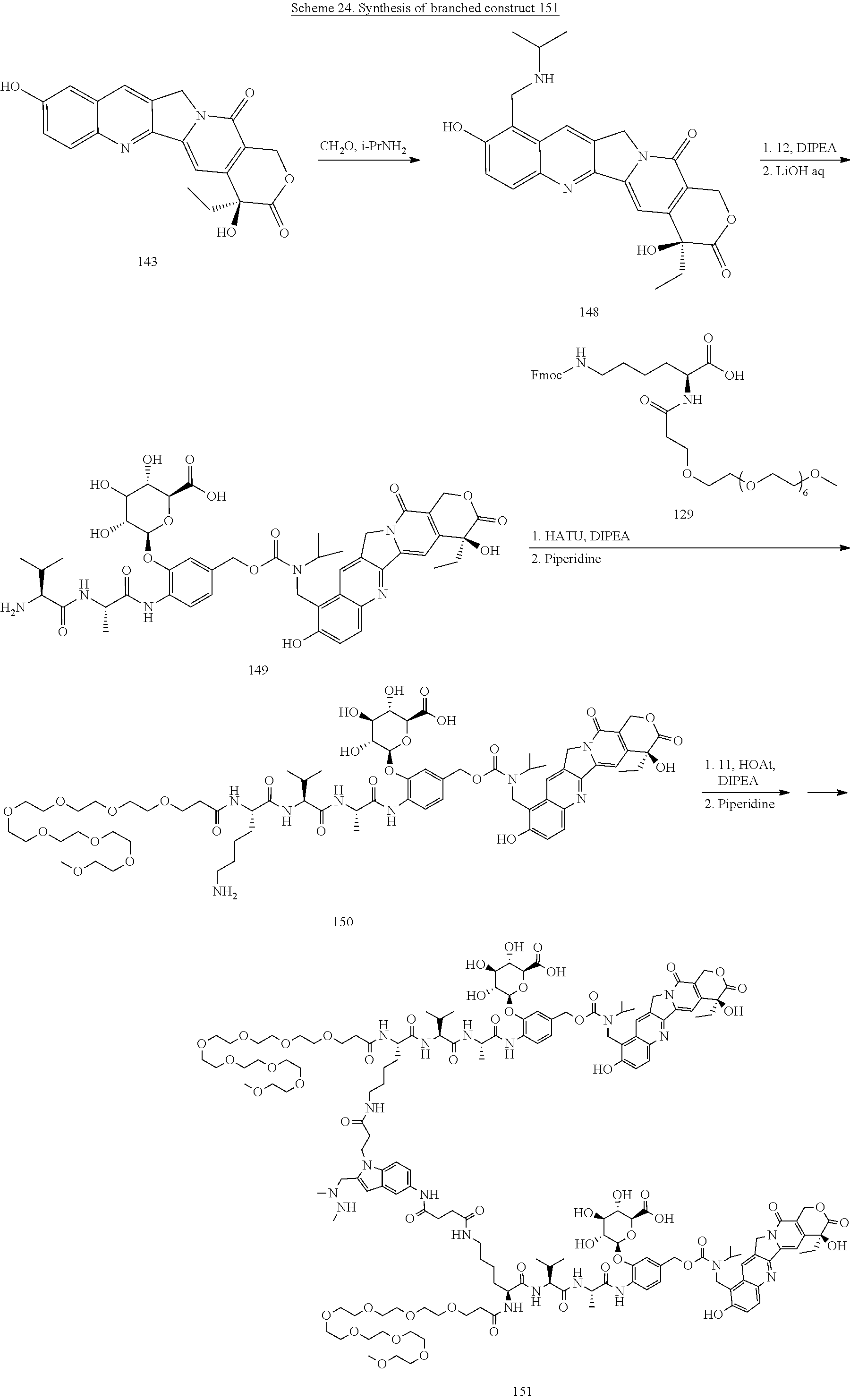

### Preparation of(S)-4-ethyl-4,9-dihydroxy-10-((isopropylamino)methyl)-1,12-dihydro-14H-pyrano[3′,4′:6,7]indolizino[1,2-b]quinoline-3,14(4H)-dione (148)

To a solution of 10-hydroxycamptothecin (500 mg, 1.37 mmol) in HOAc (30 mL) and EtOH (15 mL) were added formaldehyde (1 mL, 37 wt % in H₂O) and i-PrNH₂ (150 μL, 1.83 mmol) at room temperature. Reaction mixture was stirred overnight and then concentrated in vacuum. The residue was purified by reversed phase chromatography (C18, 0-70% v/v MeCN—H₂O with 0.05% TFA). Pure fractions were collected and lyophilized to obtain compound 148 as an orange solid (200 mg, 0.46 mmol, 36% yield). LRMS (ESI): m/z 436.2 [M+H]⁺, Calcd for C₂₄H₂₅N₃O₅ m/z 436.2.

### Preparation of (2S,3S,4S,5R,6S)-6-(2-((S)-2-((S)-2-amino-3-methylbutanamido)propanamido)-5-((((((S)-4-ethyl-4,9-dihydroxy-3,14-dioxo-3,4,12,14-tetrahydro-1H-pyrano[3′,4′:6,7]indolizino[1,2-b]quinolin-10-yl)methyl)(isopropyl)carbamoyl)oxy)methyl)phenoxy)-3,4,5-trihydroxytetrahydro-2H-pyran-2-carboxylic acid (149)

To a solution of compound 148 (50 mg, 115 μmol) in DMF (3 mL) were added HOAt (16 mg, 115 μmol) and DIPEA (60 μL, 344 μmol) at room temperature. The resulting mixture was treated with PNP-carbonate 12 (116 mg, 115 μmol) and stirred at room temperature overnight until all starting materials were consumed as judged by HPLC analysis. Reaction mixture was then diluted with water (10 mL), the resulting precipitate was collected and dissolved in THF (3 mL). The THF solution was then treated with aq. LiOH (1 mL, 1M) in at 0° C., stirred for 30 min, warmed up to room temperature, and stirred for 1 h. Reaction mixture was purified by reversed phase prep HPLC (C18, 0-70% v/v MeCN—H₂O with 0.05% TFA). Pure fractions were combined and lyophilized to give compound 149 as a yellow solid (31 mg, 33 μmol, 29% yield). LRMS (ESI): m/z 947.4 [M+H]⁺, Calcd for C₄₆H₅₄N₆O₁₆ m/z 947.4.

### Preparation of (2S,3S,4S,5R,6S)-6-(2-((28S,31S,34S)-28-(4-aminobutyl)-31-isopropyl-34-methyl-26,29,32-trioxo-2,5,8,11,14,17,20,23-octaoxa-27,30,33-triazapentatriacontan-35-amido)-5-((((((S)-4-ethyl-4,9-dihydroxy-3,14-dioxo-3,4,12,14-tetrahydro-1H-pyrano[3′,4′:6,7]indolizino[1,2-b]quinolin-10-yl)methyl)(isopropyl)carbamoyl)oxy)methyl)phenoxy)-3,4,5-trihydroxytetrahydro-2H-pyran-2-carboxylic acid (150)

To a stirred solution of carboxylic acid 129 (31 mg, 41 μmol) in anhydrous DMF (2 mL) were added HATU (15 mg, 36 μmol) and DIPEA (17 μL, 94 μmol) at room temperature. The resulting mixture was stirred for 1 h, then compound 149 (31 mg, 33 μmol) was added to the mixture, and stirring continued for 1 h. Next, reaction mixture was directly treated with piperidine (62 μL, 0.63 mmol) at room temperature, stirred for 20 minutes, and purified by reversed-phase prep HPLC (C18, 0-70% v/v MeCN—H₂O with 0.05% TFA). Pure fractions were collected and lyophilized to afford compound 150 as a yellow solid (26 mg, 18 μmol, 55% yield). LRMS (ESI): m/z 1469.7 [M+H]⁺, Calcd for C₇₀H₁₀₀N₈O₂₆ m/z 1469.7.

### Preparation of (2S,3S,4S,5R,6S)-6-(2-((28S,31S,34S)-28-(4-(3-(5-((S)-28-(((S)-1-(((S)-1-((2-(((2S,3R,4S,5S,6S)-6-carboxy-3,4,5-trihydroxytetrahydro-2H-pyran-2-yl)oxy)-4-((((((S)-4-ethyl-4,9-dihydroxy-3,14-dioxo-3,4,12,14-tetrahydro-1H-pyrano[3′,4′:6,7]indolizino[1,2-b]quinolin-10-yl)methyl)(isopropyl)carbamoyl)oxy)methyl)phenyl)amino)-1-oxopropan-2-yl)amino)-3-methyl-1-oxobutan-2-yl)carbamoyl)-26,34-dioxo-2,5,8,11,14,17,20,23-octaoxa-27,33-diazaheptatriacontan-37-amido)-2-((1,2-dimethylhydrazinyl)methyl)-1H-indol-1-yl)propanamido)butyl)-31-isopropyl-34-methyl-26,29,32-trioxo-2,5,8,11,14,17,20,23-octaoxa-27,30,33-triazapentatriacontan-35-amido)-5-((((((S)-4-ethyl-4,9-dihydroxy-3,14-dioxo-3,4,12,14-tetrahydro-1H-pyrano[3′,4′:6,7]indolizino[1,2-b]quinolin-10-yl)methyl)(isopropyl)carbamoyl)oxy)methyl)phenoxy)-3,4,5-trihydroxytetrahydro-2H-pyran-2-carboxylic acid (151)

To a solution of compound 150 (27 mg, 18 μmol) in DMF (1.5 mL) were added DIPEA (10 μL, 55 μmol) and HOAt (8 mg, 24 μmol) at room temperature, followed by the addition of bis-PFP ester 11 (8 mg, 9 μmol) in one portion. The resulting mixture was stirred for 30 minutes, then piperidine (36 μL, 0.36 mmol) was added to the mixture at room temperature. After 20 minutes, reaction mixture was purified by reversed-phase prep HPLC (C18, 0-70% v/v MeCN—H₂O with 0.05% TFA). Pure fractions were collected and lyophilized to obtain compound 151 as a yellow solid (19 mg, 5.8 μmol, 64% yield). LRMS (ESI): m/z 1640.4 [M+2H]²⁺, Calcd for C₁₅₈H₂₂₀N₂₀O₅₅ m/z: 1640.3.

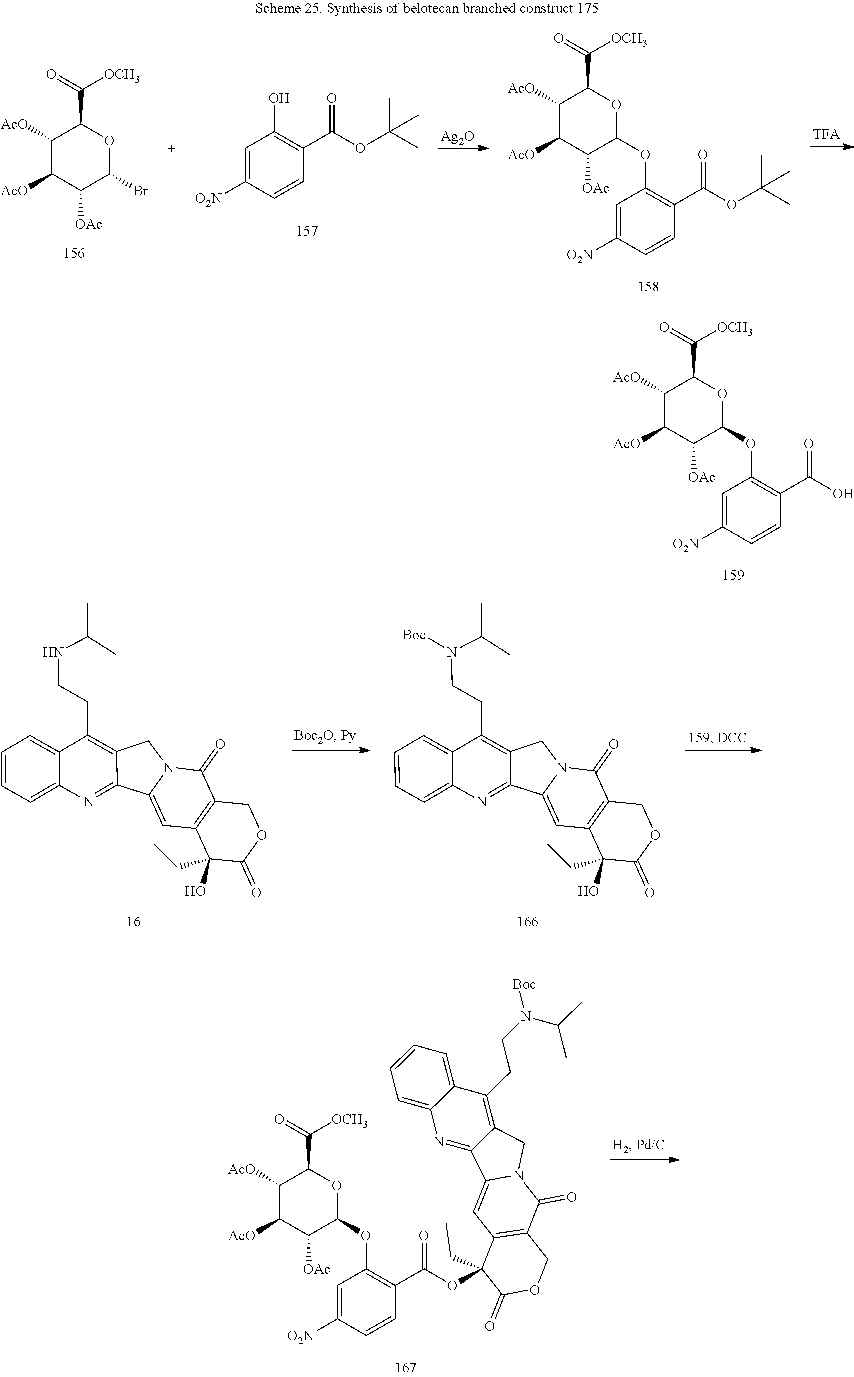

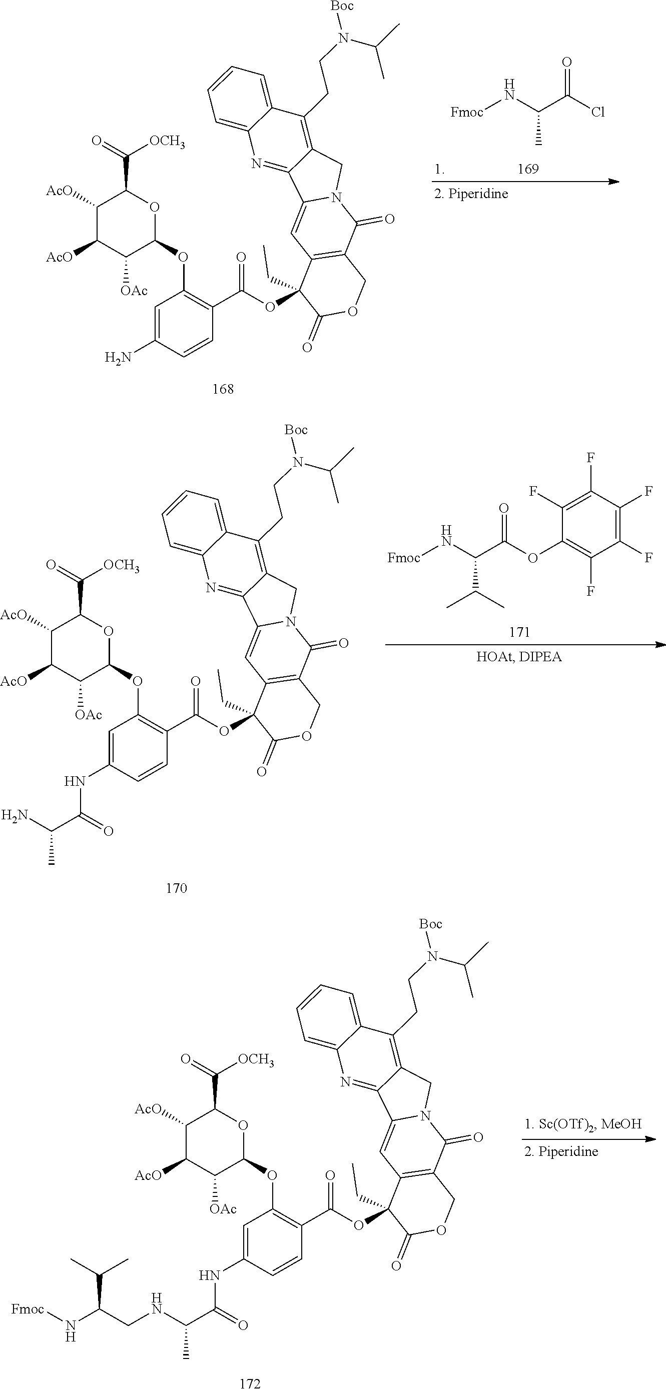

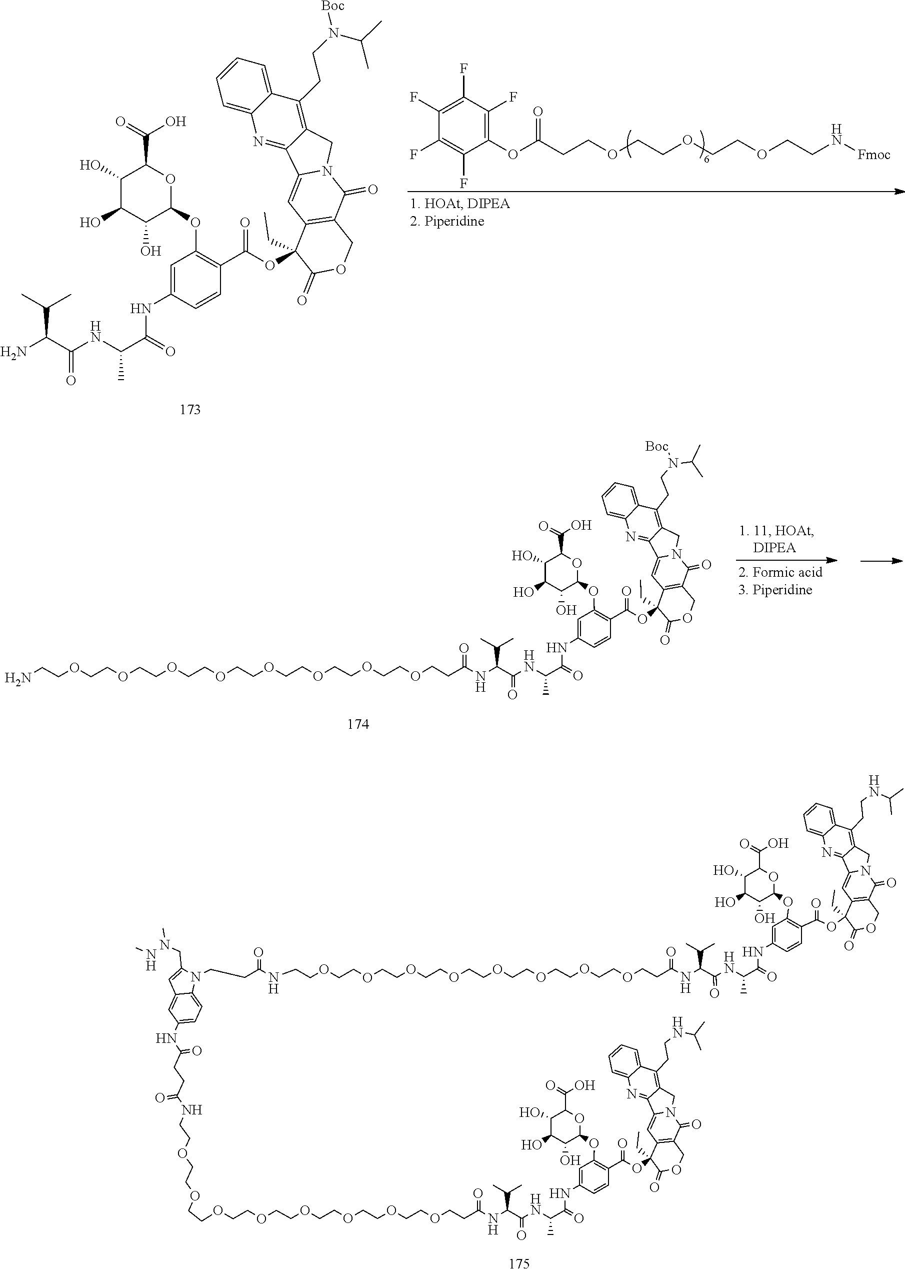

### Preparation of (2S,3R,4S,5S,6S)-2-(2-(tert-butoxycarbonyl)-5-nitrophenoxy)-6-(methoxycarbonyl)tetrahydro-2H-pyran-3,4,5-triyl triacetate (158)

To a mixture of tert-butyl 2-hydroxy-4-nitrobenzoate 157 (1.57 g, 6.6 mmol) and bromide 156 (2.37 g, 6.0 mmol) in 25 mL of acetonitrile were added silver(I) oxide (1.53 g, 6.6 mmol). The resulting mixture was stirred overnight in the dark, then filtered through a pad of silica gel, eluting with ethyl acetate, and concentrated under vacuum. The residue was purified by silica gel chromatography (0-10% EtOAc-hexane) to give 2.3 g of compound 158 as a white solid (4.1 mmol, 68% yield). LRMS (ESI): m/z 578.2 [M+Na]⁺, Calcd for C₂₄H₂₉NO₁₄ m/z 578.2.

### Preparation of 4-nitro-2-(((2S,3R,4S,5S,6S)-3,4,5-triacetoxy-6-(methoxycarbonyl)tetrahydro-2H-pyran-2-yl)oxy)benzoic acid (159)

Compound 158 (180 mg, 0.32 mmol) was dissolved in 4 mL of DCM-TFA mixture (1:1 v/v) at room temperature. The resulting solution was allowed to stand for 30 minutes, then solvents were removed under vacuum, and the residue was purified by silica gel chromatography (0-5% MeOH-DCM) to give 160 mg of carboxylic acid 159 (0.32 mmol, quant. yield) as a pink foamy solid. LRMS (ESI): m/z 522.1 [M+Na]⁺, Calcd for C₂₄H₂₉NO₁₄ m/z 522.1.

### Preparation of tert-butyl (S)-(2-(4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamate (166)

To a mixture of belotecan 2 (50 mg, 0.11 mmol) and Boc₂O (12 mg, 0.23 mmol) in dichloromethane (2 mL) were added DIPEA (40 μL, 0.23 mmol) at room temperature. After stirring for 6 hours, the reaction mixture was directly purified by silica gel chromatography (DCM-MeOH, 100:0 to 95:5 v/v) to yield compound 166 (44 mg, 0.08 mmol, 73% yield) as an off-white solid. LRMS (ESI): m/z 534.3 [M+H]⁺, Calcd for C₃₀H₃₅N₃O₆ m/z 534.3.

### Preparation of (2S,3R,4S,5S,6S)-2-(5-amino-2-((((S)-11-(2-((tert-butoxycarbonyl)(isopropyl)amino)ethyl)-4-ethyl-3,14-dioxo-3,4,12,14-tetrahydro-1H-pyrano[3′,4′:6,7]indolizino[1,2-b]quinolin-4-yl)oxy)carbonyl)phenoxy)-6-(methoxycarbonyl)tetrahydro-2H-pyran-3,4,5-triyl triacetate (168)

To a solution of carboxylic acid 159 (240 mg, 480 μmol) in dichloromethane (1 mL) and DMF (0.5 mL) were added Boc-protected belotecan 166 (100 mg, 190 μmol), followed by DCC (6 mg, 29 μmol) and DMAP (3 mg, 25 μmol) at 0° C. After 1 h, reaction mixture was allowed to warm to room temperature and stirred overnight. The mixture was briefly purified by passing through a silica gel pad (0-6% MeOH-DCM as an eluent) to give crude compound 167, which was dissolved in EtOAc (2 mL) and combined with Pd/C (10 wt %, 20 mg) and triethylamine (20 μL, 220 μmol). Reaction flask was then evacuated and filled with hydrogen gas from a balloon, in three repeating cycles. Reaction mixture was vigorously stirred for 48 h at room temperature with H₂ balloon attached, then filtered through a pad of celite. The filtrate was concentrated under vacuum and purified by silica gel chromatography (0-5% MeOH-DCM) to yield compound 168 (60 mg, 61 μmol, 33% yield) as a yellow solid. LRMS (ESI): m/z 985.4 [M+H]⁺, Calcd for C₅₀H₅₆N₄O₁₇ m/z 985.4.

### Preparation of (2S,3R,4S,5S,6S)-2-(5-((S)-2-((S)-2-((((9H-fluoren-9-yl)methoxy)carbonyl)amino)-3-methylbutanamido)propanamido)-2-((((S)-11-(2-((tert-butoxycarbonyl)(isopropyl)amino)ethyl)-4-ethyl-3,14-dioxo-3,4,12,14-tetrahydro-1H-pyrano[3′,4′:6,7]indolizino[1,2-b]quinolin-4-yl)oxy)carbonyl)phenoxy)-6-(methoxycarbonyl)tetrahydro-2H-pyran-3,4,5-triyl triacetate (172)

To a mixture of amine 168 (60 mg, 61 μmol) and Fmoc-Ala-Cl 169 (20 mg, 61 μmol) in DMF (1 mL) were added DIPEA (22 μL, 120 μmol) at room temperature. Reaction mixture was stirred for 1 h, then DMF (0.5 mL) and piperidine (50 μL) were added to the mixture. After 30 minutes, the reaction was semi-purified by silica gel chromatography with a gradient of 0 to 5% MeOH in DCM to give crude compound 170. Next, a solution of 170 in 1 mL of acetonitrile was treated with Fmoc-Val-OPFP 171 (62 mg, 120 μmol) and DIPEA (22 μL, 120 μmol) at room temperature. After stirring for 20 minutes, reaction mixture was purified by silica gel chromatography (MeOH-DCM 0-5% gradient) to yield compound 172 (70 mg, 51 μmol, 83% yield) as a yellow solid. LRMS (ESI): m/z 1377.5 [M+H]⁺, Calcd for C₇₃H₈₀N₆O₂₁ m/z 1377.5.

### Preparation of (2S,3S,4S,5R,6S)-6-(5-((S)-2-((S)-2-amino-3-methylbutanamido)propanamido)-2-((((S)-11-(2-((tert-butoxycarbonyl)(isopropyl)amino)ethyl)-4-ethyl-3,14-dioxo-3,4,12,14-tetrahydro-1H-pyrano[3′,4′:6,7]indolizino[1,2-b]quinolin-4-yl)oxy)carbonyl)phenoxy)-3,4,5-trihydroxytetrahydro-2H-pyran-2-carboxylic acid (173)

To a solution of compound 172 (70 mg, 51 μmol) in a MeOH—H₂O mixture (4:1 v/v, 1 mL) were added Sc(OTf)₃ (640 mg, 1.3 mmol) at room temperature. Reaction mixture was stirred for two days, then concentrated, and reconstituted in DMF-piperidine mixture (10:1 v/v, 1.1 mL). Reaction mixture was stirred for 1 hour and purified by reversed-phase chromatography on C18 column (H₂O/CH₃CN with 0.05% TFA, 90:10 to 20:80 v/v) to compound 173 (5 mg, 5 μmol, 10% yield). LRMS (ESI): m/z 1015.4 [M+H]⁺, Calcd for C₅₁H₆₂N₆O₁₆ m/z 1015.4.

### Preparation of (2S,3S,4S,5R,6S)-6-(5-((29S,32S)-1-amino-29-isopropyl-32-methyl-27,30-dioxo-3,6,9,12,15,18,21,24-octaoxa-28,31-diazatritriacontan-33-amido)-2-((((S)-11-(2-((tert-butoxycarbonyl)(isopropyl)amino)ethyl)-4-ethyl-3,14-dioxo-3,4,12,14-tetrahydro-1H-pyrano[3′,4′:6,7]indolizino[1,2-b]quinolin-4-yl)oxy)carbonyl)phenoxy)-3,4,5-trihydroxytetrahydro-2H-pyran-2-carboxylic acid (174)

To a mixture of amine 174 (5 mg, 5 μmol) and PFP ester 125 (12 mg, 6 μmol) in DMF (0.5 mL) were added DIPEA (5 μL, 29 μmol) at room temperature. Reaction mixture was stirred for 1 hour, then DMF (0.5 mL) and piperidine (50 μL) were added to the mixture. After stirring for 15 minutes at room temperature, the reaction mixture was directly purified by reversed phase prep HPLC using C18 column (H₂O/CH₃CN with 0.05% TFA, 90:10 to 30:70 v/v). Fractions containing the desired compound were pooled and lyophilized to yield compound 174 (2 mg, 1.4 μmol, 28% yield). LRMS (ESI): m/z 1438.7 [M+H]⁺, Calcd for C₇₀H₉₉N₇O₂₅ m/z 1438.7.

### Preparation of (2S,3S,4S,5R,6S)-6-(5-((2S,5S)-38-((1-((2S,5S)-1-((3-(((2S,3R,4S,5S,6S)-6-carboxy-3,4,5-trihydroxytetrahydro-2H-pyran-2-yl)oxy)-4-((((S)-4-ethyl-11-(2-(isopropylamino)ethyl)-3,14-dioxo-3,4,12,14-tetrahydro-1H-pyrano[3′,4′:6,7]indolizino[1,2-b]quinolin-4-yl)oxy)carbonyl)phenyl)amino)-5-isopropyl-2-methyl-1,4,7,35-tetraoxo-10,13,16,19,22,25,28,31-octaoxa-3,6,34-triazaheptatriacontan-37-yl)-2-((1,2-dimethylhydrazineyl)methyl)-1H-indol-5-yl)amino)-5-isopropyl-2-methyl-4,7,35,38-tetraoxo-10,13,16,19,22,25,28,31-octaoxa-3,6,34-triazaoctatriacontanamido)-2-((((S)-4-ethyl-11-(2-(isopropylamino)ethyl)-3,14-dioxo-3,4,12,14-tetrahydro-1H-pyrano[3′,4′:6,7]indolizino[1,2-b]quinolin-4-yl)oxy)carbonyl)phenoxy)-3,4,5-trihydroxytetrahydro-2H-pyran-2-carboxylic acid (175)

To a stirred mixture of amine 174 (2 mg, 1.4 μmol) and bis-PFP-ester 11 (0.8 mg, 0.7 μmol) in DMF (1 mL) were added DIPEA (0.5 μL, 2.8 μmol) at room temperature. After 2 hours, reaction mixture was concentrated and then reconstituted in formic acid (1 mL) at room temperature. After 30 minutes, formic acid was removed in vacuum, and the residue was reconstituted in DMF (1 mL) and piperidine (50 μL). After stirring for 15 minutes at room temperature, the reaction mixture was directly purified by reversed phase HPLC using C₁₈ column (H₂O/CH₃CN with 0.05% TFA, 90:10 to 35:65 v/v). Fractions containing the desired compound were pooled and lyophilized to yield compound 175 (0.7 mg, 0.2 μmol, 33% yield) as a yellow powder. LRMS (ESI): m/z 1509.2 [M+2H]²⁺, Calcd for C₁₄₈H₂₀₂N₁₈O₄₉ m/z 1508.7.

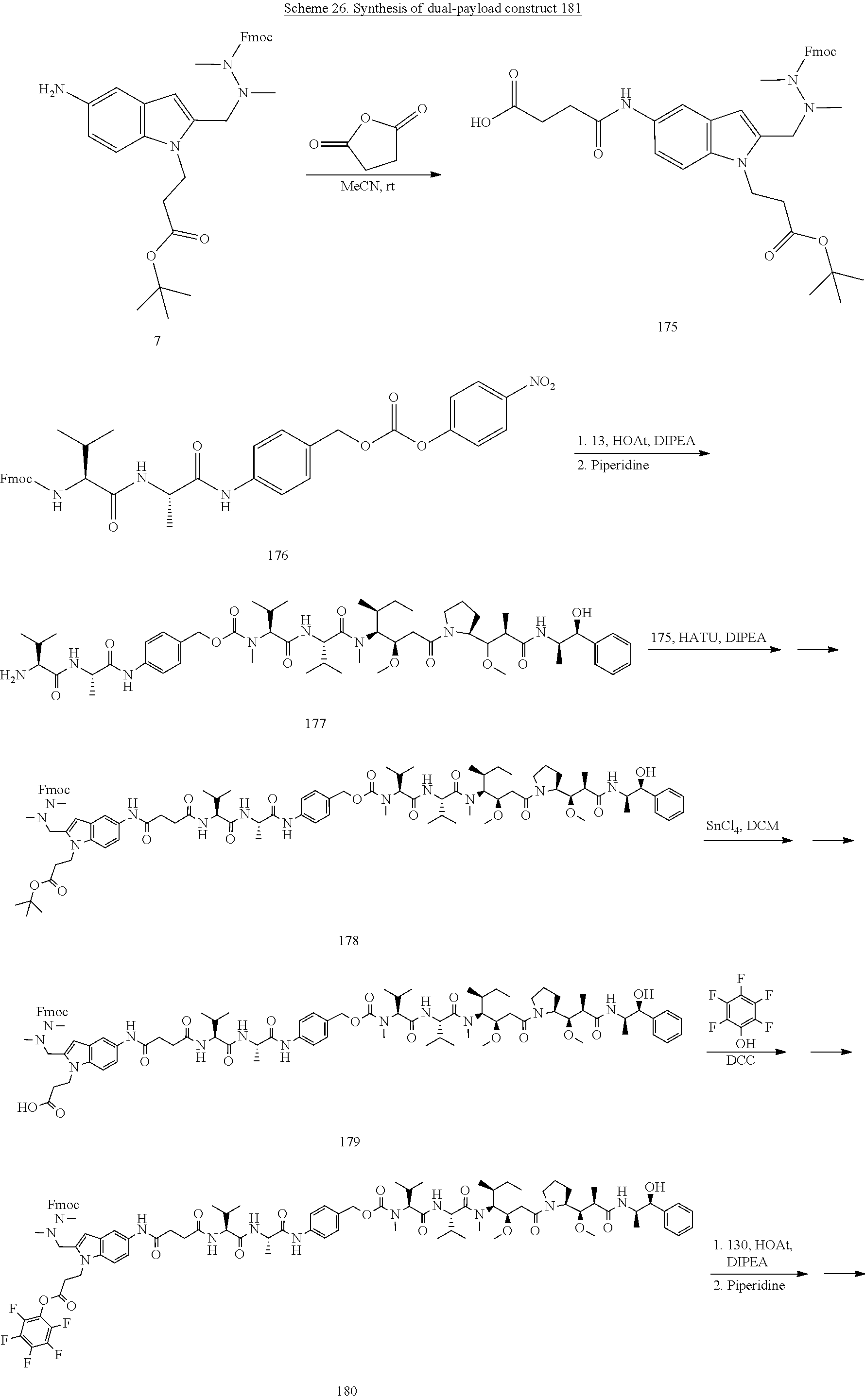

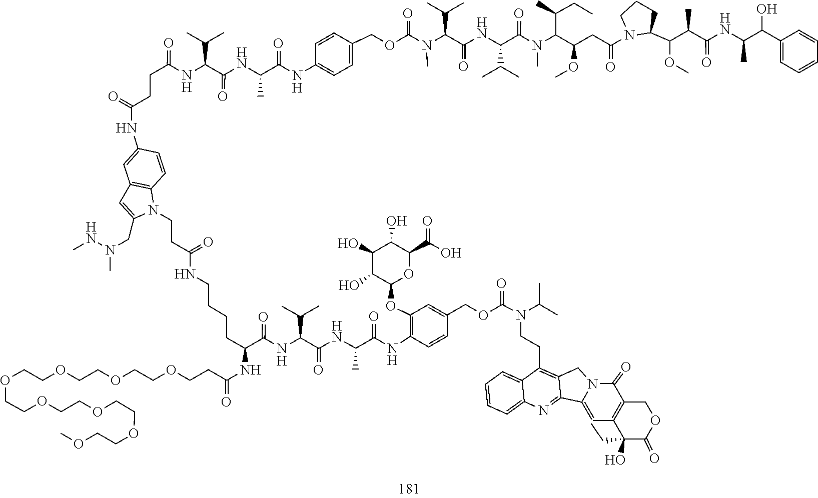 [SMILES: CC[C@H](C)C([C@@H](CC(=O)N1CCC[C@H]1C(OC)[C@@H](C)C(=O)N[C@H](C)C(O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCC(=O)Nc2ccc3c(c2)cc(CN(C)NC)n3CCC(=O)NCCCC[C@H](NC(=O)CCOCCOCCOCCOCCOCCOCCOCCOC)C(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)cc1)C(C)C]

### Preparation of 4-((2-((2-(((9H-fluoren-9-yl)methoxy)carbonyl)-1,2-dimethylhydrazineyl)methyl)-1-(3-(tert-butoxy)-3-oxopropyl)-1H-indol-5-yl)amino)-4-oxobutanoic acid (175)

To a solution of free amine (450 mg, 0.81 mmol) in acetonitrile (5 mL) were added succinic anhydride (405 mg, 4.1 mmol) at room temperature. Reaction mixture was allowed to stir for 1 h until judged complete by LCMS analysis and directly purified by reversed phase chromatography (C18, 0-100% CH₃CN—H₂O with 0.05% TFA). Pure fractions were collected and lyophilized to obtain compound 175 as a brown solid (397 mg, 0.61 mmol, 75% yield). LRMS (ESI): m/z 655.3 [M+H]⁺, Calcd for C₃₇H₄₂N₄O₇ m/z 655.3.

### Preparation of 4-((S)-2-((S)-2-amino-3-methylbutanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (177)

To a mixture of PNP-carbonate 176 (100 mg, 0.15 mmol) and MMAE (106 mg, 147 L) in anhydrous DMF (1 mL) were added HOAt (20 mg, 0.15 mmol) and DIPEA (77 μL, 0.44 mmol). Reaction mixture was stirred overnight at room temperature until all the starting materials were consumed. Piperidine (290 μL, 2.94 mmol) was then added to the reaction mixture. After 30 minutes, reaction mixture was quenched with 1M HCl to slightly acidic pH. The mixture was filtered and purified by reversed phase chromatography (C18, 0-70% CH₃CN—H₂O with 0.05% TFA). The pure fractions were collected and lyophilized to obtain compound 177 as a yellow solid (113 mg, 109 μmol, 74% yield). LRMS (ESI): m/z 1037.7 [M+H]⁺, Calcd for C₅₅H₈₈N₈O₁₁ m/z 1037.7.

### Preparation of (9H-fluoren-9-yl)methyl 2-((1-(3-(tert-butoxy)-3-oxopropyl)-5-(4-(((S)-1-(((S)-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1- methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxopropan-2-yl)amino)-3-methyl-1-oxobutan-2-yl)amino)-4-oxobutanamido)-1H-indol-2-yl)methyl)-1,2-dimethylhydrazine-1-carboxylate (178)

To a mixture of compound 175 (63 mg, 96 μmol) and 177 (100 mg, 96 μmol) in DMF (2 mL) were added HATU (36 mg, 96 μmol) and DIPEA (50 μL, 288 μmol) and the resulting solution was stirred for 1 h at room temperature. Reaction mixture was purified by reversed phase chromatography (C18, 0-100% CH₃CN—H₂O with 0.05% TFA). Pure fractions were collected and lyophilized to obtain compound 178 as a yellow solid (160 mg, 94 μmol, 98% yield). LRMS (ESI): m/z 1696.7 [M+Na]⁺, Calcd for C₉₂H₁₂₈N₁₂O₁₇ m/z: 1696.9.

### Preparation of 3-(2-((2-(((9H-fluoren-9-yl)methoxy)carbonyl)-1,2-dimethylhydrazineyl)methyl)-5-(4-(((S)-1-(((S)-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin--yl)-2-oxoethyl)-5,8-diisopropyl-4,10-dimethyl-3,6,9-trioxo-2,13-dioxa-4,7,10-triazatetradecyl)phenyl)amino)-1-oxopropan-2-yl)amino)-3-methyl-1-oxobutan-2-yl)amino)-4-oxobutanamido)-1H-indol-1-yl)propanoic acid (179)

To a solution of compound 178 (100 mg, 60 μmol) in anhydrous DCM (1 mL) were added SnCl₄ solution (0.6 mL, 1M in DCM) at ambient temperature. After 30 minutes, reaction mixture was quenched with water (1 mL). The product was extracted with EtOAc (10 mL). Organic layer was washed with brine and dried over sodium sulfate. After solvents were removed in vacuum, the crude residue was purified by reversed phase chromatography (C18, 0-100% CH₃CN—H₂O with 0.05% TFA). Pure fractions were collected and lyophilized to obtain compound 179 as a white solid (40 mg, 25 μmol, 42% yield). LRMS (ESI): m/z 1617.9 [M+H]⁺, Calcd for C₈₈H₁₂₀N₁₂O₁₇ m/z 1617.9.

### Preparation of (9H-fluoren-9-yl)methyl 2-((5-(4-(((S)-1-(((S)-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxopropan-2-yl)amino)-3-methyl-1-oxobutan-2-yl)amino)-4-oxobutanamido)-1-(3-oxo-3-(perfluorophenoxy)propyl)-1H-indol-2-yl)methyl)-1,2-dimethylhydrazine-1-carboxylate (180)

To a solution of compound 179 (12 mg, 7.4 μmol) in anhydrous THF (2 mL) were added DCC (200 mg, 0.97 mmol) and pentafluoro phenol (200 mg, 1.1 mmol) at room temperature. The resulting mixture was stirred overnight, filtered, and concentrated under vacuum. The residue was purified by reversed phase chromatography (C18, 0-100% CH₃CN—H₂O with 0.05% TFA). Pure fractions were combined and lyophilized to give compound 180 as a white solid (9 mg, 68% yield). LRMS (ESI): m/z 1783.9 [M+H]⁺, Calcd for C₉₄H₁₁₉F₅N₁₂O₁₇ m/z 1783.9.

### Preparation of (2S,3S,4S,5R,6S)-6-(2-((28S,31S,34S)-28-(4-(3-(5-(4-(((S)-1-(((S)-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxopropan-2-yl)amino)-3-methyl-1-oxobutan-2-yl)amino)-4-oxobutanamido)-2-((1,2-dimethylhydrazineyl)methyl)-1H-indol-1-yl)propanamido)butyl)-31-isopropyl-34-methyl-26,29,32-trioxo-2,5,8,11,14,17,20,23-octaoxa-27,30,33-triazapentatriacontan-35-amido)-5-((((2-((S)-4-ethyl-4-hydroxy-3,14-dioxo-3,4,12,14-tetrahydro-1H-pyrano[3′,4′:6,7]indolizino[1,2-b]quinolin-ii-yl)ethyl)(isopropyl)carbamoyl)oxy)methyl)phenoxy)-3,4,5-trihydroxytetrahydro-2H-pyran-2-carboxylic acid (181)

To a stirred solution of compound 130 (10 mg, 6.8 μmol) in DMF (1 mL) were added HOAt (1 mg, 6.8 μmol) and DIPEA (3.5 μL, 20 μmol), followed by PFP-ester 180 (9 mg, 5.0 mol) at room temperature. Reaction mixture was stirred for 1 h, then piperidine (10 μL, 100 mol) was added and stirring continued at room temperature for 30 mins. Reaction mixture was quenched with 1M HCl until slightly acidic. The mixture was then purified by reversed phase chromatography (C18, 0-70% CH₃CN—H₂O with 0.05% TFA). Pure fractions were collected and lyophilized to give compound 181 as a white solid (9 mg, 3 μmol, 60% yield). LRMS (ESI): m/z 1423.4 [M+2H]²⁺, Calcd for C₁₄₄H₂₁₀N₂₀O₃₉ m/z 1423.3.

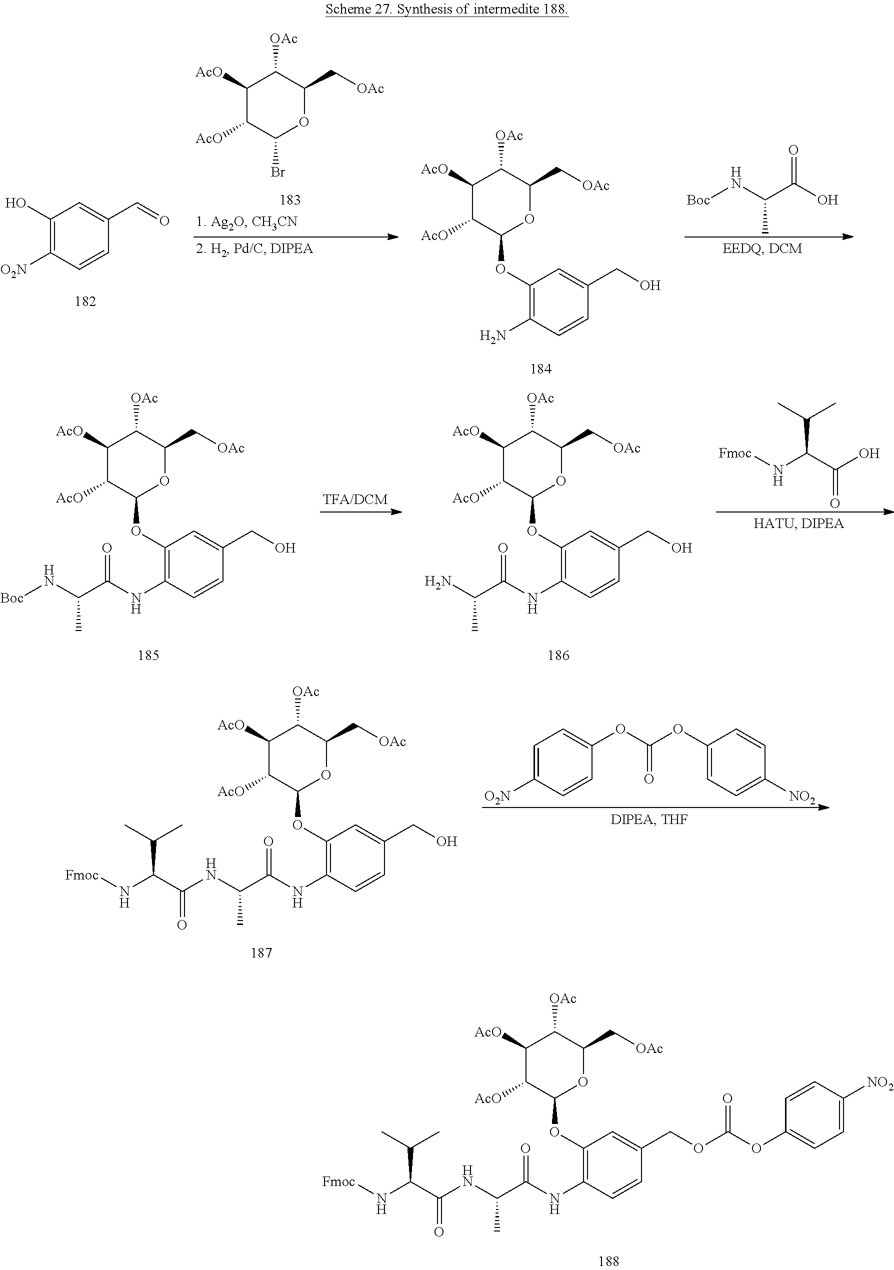

### Preparation of (2R,3R,4S,5R,6S)-2-(acetoxymethyl)-6-(2-amino-5-(hydroxymethyl)phenoxy)tetrahydro-2H-pyran-3,4,5-triyl triacetate (184)

To a mixture of compound 182 (5.0 g, 29.9 mmol, 1.7 eq.) and compound 183 (7.23 g, 17.6 mmol, 1 eq.) in anhydrous acetonitrile (100 mL) were added silver(I) oxide (15.6 g, 87.9 mmol, 5 eq.). The mixture was stirred at 25° C. in the dark for 24 hours under nitrogen. Reaction mixture was diluted with EtOAc (100 mL), filtered and concentrated under reduced pressure. The residue was purified by silica gel chromatography (0-30% hexane-EtOAc) to intermediate aldehyde.

To a mixture of intermediate aldehyde (5.61 g, 11.3 mmol) and triethylamine (2.5 mL) in EtOAc (80 mL) were added palladium on carbon (10 wt. %, 800 mg, 0.75 mmol) in one portion. The reaction mixture was stirred at 25° C. for 24 h under H₂ atmosphere. The solids were filtered off, and the resulting filtrate was concentrated to give 5.2 g (11.1 mmol, 98% yield) of product 184 as a white solid, which was used into next step without further purification.

### Preparation of (2R,3R,4S,5R,6S)-2-(acetoxymethyl)-6-(2-((S)-2-((tert-butoxycarbonyl)amino)propanamido)-5-(hydroxymethyl)phenoxy)tetrahydro-2H-pyran-3,4,5-triyl triacetate (185)

A mixture of compound 184 (5.20 g, 11.1 mmol), Boc-L-Ala-OH (1.75 g, 9.25 mmol) and EEDQ (2.3 g, 9.25 mmol) in anhydrous DCM (40 mL) and MeOH (4 mL) was stirred at room temperature in the dark for 1 h. The reaction mixture was concentrated to give 5.5 g of crude product 185 as a yellow solid. The crude product was used in the next step without further purification.

### Preparation of (2R,3R,4S,5R,6S)-2-(acetoxymethyl)-6-(2-((S)-2-aminopropanamido)-5-(hydroxymethyl) phenoxy)tetrahydro-2H-pyran-3,4,5-triyl triacetate (186)

Crude compound 185 (5.5 g, 8.6 mmol) was dissolved in TFA (23 mL). The resulting solution was stirred at room temperature for 10 min and concentrated in vacuum. The residue was purified by reversed-phase chromatography (C18 column, 0-75% acetonitrile-water with 0.05% TFA). Pure fractions were combined and concentrated to give 4.0 g of product amine 186 as a yellow oil (7.5 mmol, 66% yield over 3 steps).

### Preparation of (2S,3R,4S,5R,6R)-2-(2-((S)-2-((S)-2-((((9H-fluoren-9-yl)methoxy)carbonyl)amino)-3-methylbutanamido)propanamido)-5-(hydroxymethyl)phenoxy)-6-(acetoxymethyl)tetrahydro-2H-pyran-3,4,5-triyl triacetate (187)

To a mixture of Fmoc-L-valine (3.1 g, 9.2 mmol) and DIPEA (3.9 mL, 22.2 mmol) in anhydrous DMF (20 mL) were added HATU (3.5 g, 9.2 mmol) in one portion at room temperature. The resulting solution was stirred at room temperature for 30 min and then combined with amine 186 (4.0 g, 7.5 mmol). Reaction mixture was stirred for 16 hours and concentrated in vacuum. The residue was purified by silica gel chromatography (hexane:EtOAc, 0-100%) to give compound 187 (4.5 g, 5.2 mmol, 70% yield) as a white solid.

### Preparation of (2S,3R,4S,5R,6R)-2-(2-((S)-2-((S)-2-((((9H-fluoren-9-yl)methoxy)carbonyl)amino)-3-methylbutanamido)propanamido)-5-((((4-nitrophenoxy)carbonyl)oxy)methyl)phenoxy)-6-(acetoxymethyl)tetrahydro-2H-pyran-3,4,5-triyl triacetate (188)

To a mixture of alcohol 187 (4.5 g, 5.2 mmol) and DIPEA (4.5 mL, 26.1 mmol, 5 equiv) in anhydrous THF (20 mL) were added bis(4-nitrophenyl) carbonate (7.9 g, 26.1 mmol, 5 equiv). The resulting mixture was stirred at room temperature for 24 h and concentrated under. The residue was purified by reversed-phase chromatography (acetonitrile-water 0-70% with 0.05% TFA) to give 4-nitrophenyl carbonate product 188 as a white solid (3.9 g, 73% yield). LRMS (ESI): m/z 1027.3 [M+H]⁺, Calcd for C₅₁H₅₄N₄O₁₉ m/z 1027.3.

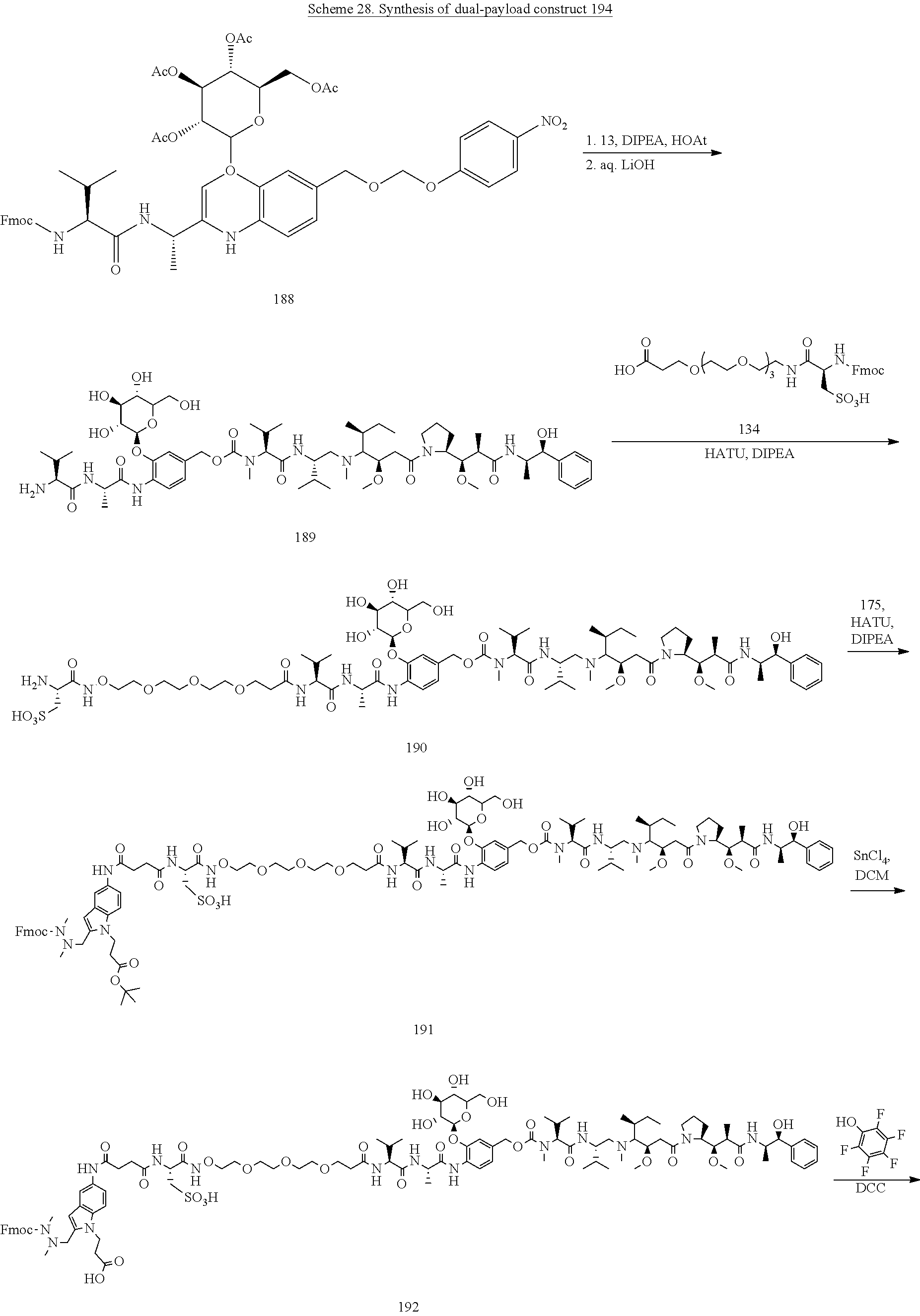

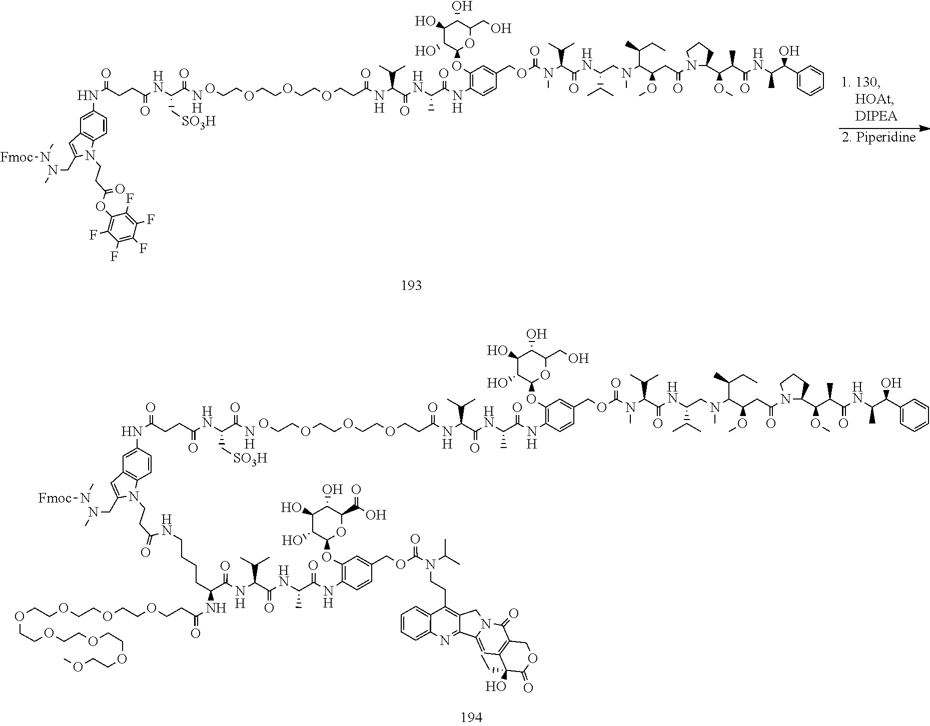

### Preparation of 4-((S)-2-((S)-2-amino-3-methylbutanamido)propanamido)-3-(((2S,3R,4S,5S,6R)-3,4,5-trihydroxy-6-(hydroxymethyl)tetrahydro-2H-pyran-2-yl)oxy)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (189)

To a mixture of MMAE (13, 50 mg, 70 μmol) and PNP carbonate 188 (72 mg, 70 μL) in anhydrous DMF (1 mL) were added HOAt (10 mg, 74 μmol) and DIPEA (36 μL, 210 μmol) at room temperature. The resulting mixture was stirred overnight and concentrated under vacuum. The residue was redissolved in THF (2 mL) and treated with aqueous LiOH solution (1M, 1 mL) at 0° C. Reaction mixture was stirred for 1 h, then allowed to warm up to room temperature and stirred for 1 h until hydrolysis was judged complete by HPLC analysis. Reaction mixture was quenched with 1M HCl, filtered, and purified by reversed-phase chromatography (C18 column, 0-70% acetonitrile-water with 0.05% TFA). Pure fractions were collected and lyophilized to give compound 189 as a yellow solid (76 mg, 90% yield). LRMS (ESI): m/z 1215.7 [M+H]⁺, Calcd for C₆₁H₉₈N₈O₁₇ m/z 1215.7.

### Preparation of (2S,5S,24R)-24-amino-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((2S,3R,4S,5S,6R)-3,4,5-trihydroxy-6-(hydroxymethyl)tetrahydro-2H-pyran-2-yl)oxy)phenyl)amino)-5-isopropyl-2-methyl-1,4,7,23-tetraoxo-10,13,16,19-tetraoxa-3,6,22-triazapentacosane-25-sulfonic acid (190)

To a solution of carboxylic acid 134 (50 mg, 78 μmol) in DMF (1 mL) were added HATU (30 mg, 78 μmol) and DIPEA (41 μL, 234 μmol) at room temperature. The resulting mixture was stirred for 20 min and then combined with compound 189 (95 mg, 78 μmol). After 1 h, piperidine (156 uL, 1.56 mmol) was added directly to the reaction mixture. After 30 min, reaction mixture was quenched with 1M HCl until slightly acidic, filtered, and purified by reversed-phase prep HPLC (C18 column, 0-70% acetonitrile-water with 0.05% TFA). Pure fractions were combined and lyophilized to give product 190 as a white solid (100 mg, 79% yield). LRMS (ESI): m/z 1613.8 [M+Na]⁺, Calcd for C₇₅H₁₂₄N₁₀O₂₆S m/z 1613.8.

### Preparation of (2S,5S,24R)-24-(4-((2-((2-(((9H-fluoren-9-yl)methoxy)carbonyl)-1,2-dimethylhydrazineyl)methyl)-1-(3-(tert-butoxy)-3-oxopropyl)-1H-indol-5-yl)amino)-4-oxobutanamido)-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((2S,3R,4S,5S,6R)-3,4,5-trihydroxy-6-(hydroxymethyl)tetrahydro-2H-pyran-2-yl)oxy)phenyl)amino)-5-isopropyl-2-methyl-1,4,7,23-tetraoxo-10,13,16,19-tetraoxa-3,6,22-triazapentacosane-25-sulfonic acid (191)

To a mixture of carboxylic acid 175 (20 mg, 30 μmol) and compound 190 (50 mg, 31 mol) in anhydrous DMF (2 mL) were added HATU (12 mg, 31 μmol) and DIPEA (16 μL, 288 mol). Reaction mixture was stirred for 1 h at room temperature and then directly purified by reversed-phase prep HPLC (C18 column, 0-70% acetonitrile-water with 0.05% TFA). Pure fractions were collected and lyophilized to give compound 191 as a white solid (20 mg, 29% yield). LRMS (ESI): m/z 1126.1 [M+2H]²⁺, Calcd for C₁₁₂H₁₆₄N₁₄O₃₂S m/z 1126.1.

### Preparation of 3-(2-((2-(((9H-fluoren-9-yl)methoxy)carbonyl)-1,2-dimethylhydrazineyl)methyl)-5-((2S,5S,24R)-1-((4-((5S,8S,11S,12R)-11-((S)-sec-butyl)-12-(2-((S)-2-((1R,2R)-3-(((i S,2R)-1-hydroxy-1-phenylpropan-2-yl)amino)-1-methoxy-2-methyl-3-oxopropyl)pyrrolidin--yl)-2-oxoethyl)-5,8-diisopropyl-4,10-dimethyl-3,6,9-trioxo-2,13-dioxa-4,7,10-triazatetradecyl)-2-(((2S,3R,4S,5S,6R)-3,4,5-trihydroxy-6-(hydroxymethyl)tetrahydro-2H-pyran-2-yl)oxy)phenyl)amino)-5-isopropyl-2-methyl-1,4,7,23,26-pentaoxo-24-(sulfomethyl)-10,13,16,19-tetraoxa-3,6,22,25-tetraazanonacosan-29-amido)-1H-indol-1-yl)propanoic acid (192)

To a solution of compound 191 (20 mg, 9 μmol) in DCM (1 mL) was added SnCl₄ solution (2 mL, 1M in DCM) at room temperature. After 30 minutes, reaction mixture was quenched with water (1 mL) and extracted with EtOAc (10 mL). Organic layer was washed with brine, dried over sodium sulfate, and concentrated under reduced pressure. The residue was purified by reversed-phase prep HPLC (C18 column, 0-70% acetonitrile-water with 0.05% TFA). Pure fractions were pooled and lyophilized to obtain compound 192 as a white solid (10 mg, 52% yield). LRMS (ESI): m/z 1098.1 [M+2H]²⁺, Calcd for C₁₀₈H₁₅₆N₁₄O₃₂S m/z 1098.0.

### Preparation of (2S,5S,24R)-24-(4-((2-((2-(((9H-fluoren-9-yl)methoxy)carbonyl)-1,2-dimethylhydrazineyl)methyl)-1-(3-oxo-3-(perfluorophenoxy)propyl)-1H-indol-5-yl)amino)-4-oxobutanamido)-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((2S,3R,4S,5S,6R)-3,4,5-trihydroxy-6-(hydroxymethyl)tetrahydro-2H-pyran-2-yl)oxy)phenyl)amino)-5-isopropyl-2-methyl-1,4,7,23-tetraoxo-10,13,16,19-tetraoxa-3,6,22-triazapentacosane-25-sulfonic acid (193)

To a solution of compound 192 (10 mg, 4.6 μmol) in THF (1 mL) were added DCC (94 mg, 460 μmol) and pentafluorophenol (85 mg, 460 mmol) at room temperature. Reaction mixture was stirred overnight, filtered, and purified by reversed-phase chromatography (C₁₈ column, 0-100% acetonitrile-water with 0.05% TFA). Pure fractions were collected and lyophilized to obtain PFP ester 193 as a white solid (8 mg, 74% yield). LRMS (ESI): m/z 1181.1 [M+2H]²⁺, Calcd for C₁₁₄H₁₅₅F₅N₁₄O₃₂S m/z 1181.0.

### Preparation of (2S,3S,4S,5R,6S)-6-(2-((28S,31S,34S)-28-(4-(3-(5-((2S,5S,24R)-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((2S,3R,4S,5S,6R)-3,4,5-trihydroxy-6-(hydroxymethyl)tetrahydro-2H-pyran-2-yl)oxy)phenyl)amino)-5-isopropyl-2-methyl-1,4,7,23,26-pentaoxo-24-(sulfomethyl)-10,13,16,19-tetraoxa-3,6,22,25-tetraazanonacosan-29-amido)-2-((1,2-dimethylhydrazineyl)methyl)-1H-indol-1-yl)propanamido)butyl)-31-isopropyl-34-methyl-26,29,32-trioxo-2,5,8,11,14,17,20,23-octaoxa-27,30,33-triazapentatriacontan-35-amido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (194)

To a solution of compound 130 (8 mg, 5.5 μmol) in DMF (1 mL) were added HOAt (1 mg, 7.4 mmol) and DIPEA (2.8 μL, 17 μmol), followed by PFP-ester 193 (12 mg, 5.0 μmol) at room temperature. The resulting mixture was stirred for 1 h, then piperidine was added and stirring continued for 30 mins. Reaction mixture was quenched by addition of aqueous 1M HCl until slightly acidic, then purified by reversed-phase prep HPLC (C18 column, 0-70% acetonitrile-water with 0.05% TFA). Pure fractions were collected and lyophilized to obtain compound 194 as a white solid (4 mg, 24% yield). LRMS (ESI−): m/z 1709.5 [M−2H]²⁻, Calcd for C₁₆₄H₂₄₆N₂₂O₅₄S m/z 1709.3.

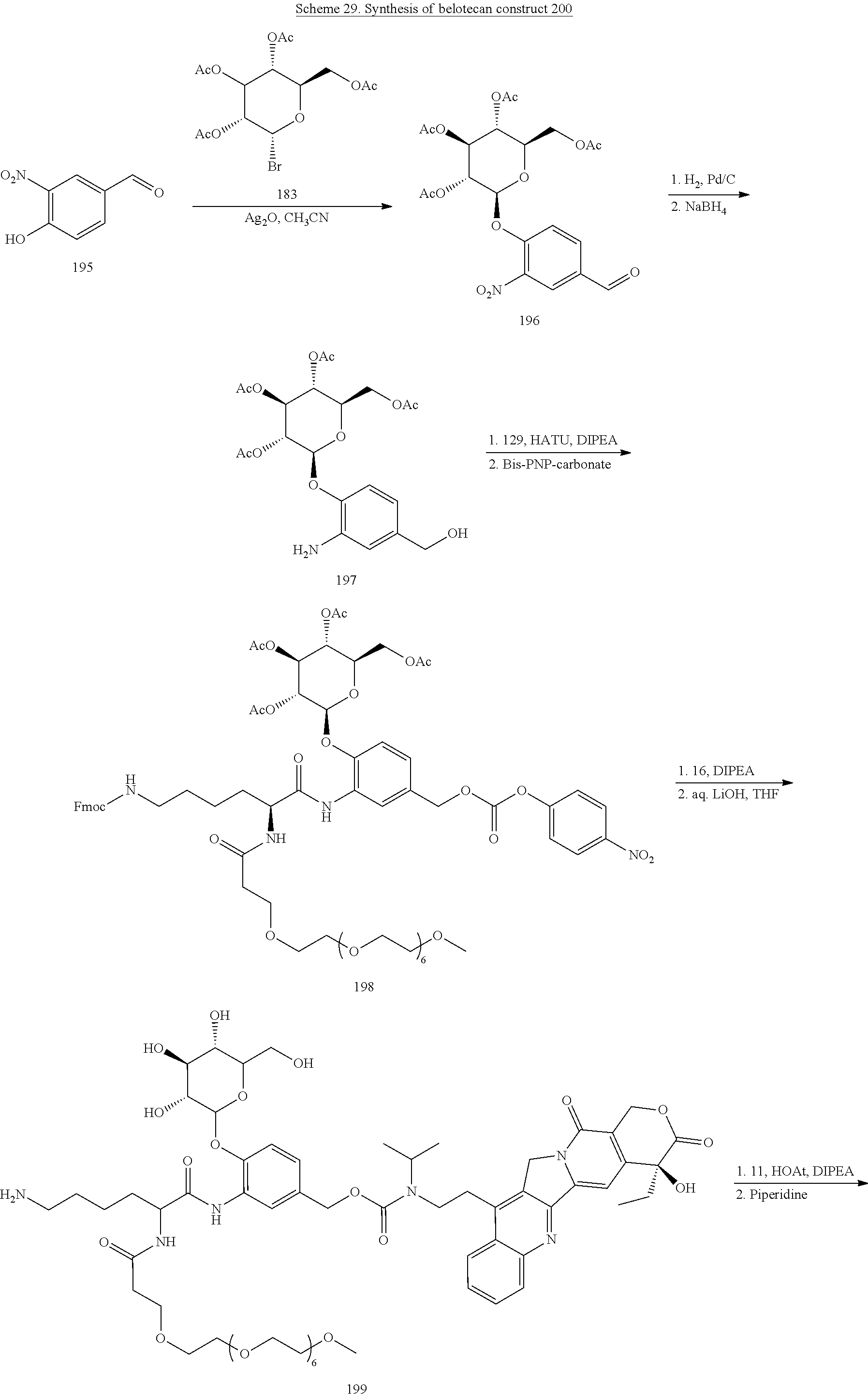

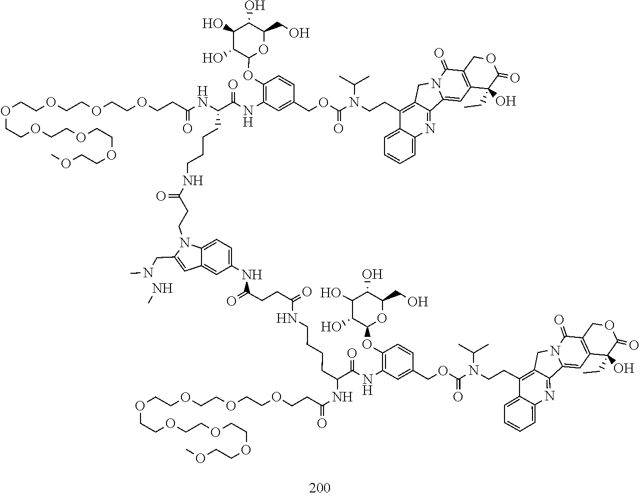

### Preparation of (2R,3R,4S,5R,6S)-2-(acetoxymethyl)-6-(4-formyl-2-nitrophenoxy)tetrahydro-2H-pyran-3,4,5-triyl triacetate (196)

To a mixture of nitrobenzaldehyde 195 (0.59 g, 3.5 mmol) and bromide 183 (2.0 g, 4.7 mmol) in 40 mL of acetonitrile were added silver(I) oxide (2.5 g, 10.8 mmol) at room temperature. Reaction mixture was stirred for 24 h in the dark, filtered through a plug of silica gel and concentrated under vacuum. The residue was purified by silica gel chromatography (10-90% EtOAc-hexane) to afford compound 196 (1.5 g, 3.0 mmol, 86% yield) as a light yellowish solid. LRMS (ESI): m/z 520.2 [M+Na]-, Calcd for C₂₁H₂₃NO₁₃ m/z 520.1.

### Preparation of (2R,3R,4S,5R,6S)-2-(acetoxymethyl)-6-(2-amino-4-(hydroxymethyl)phenoxy)tetrahydro-2H-pyran-3,4,5-triyl triacetate (197)

To a mixture of nitro compound 196 (1.43 g, 2.9 mmol) in 5 mL of ethyl acetate were added palladium on carbon (10% wt., 80 mg) and 80 μL of triethylamine. Air was removed from the reaction flask and hydrogen balloon was connected. Reaction mixture was stirred overnight at room temperature, then filtered through a pad of celite and concentrated under vacuum. The residue was reconstituted in 4 mL of anhydrous chloroform and 1 mL of isopropanol. To this solution were added 120 mg of silica gel and 100 mg (2.6 mmol) of sodium borohydride at 0° C. Reaction mixture was allowed to warm up to room temperature, stirred for 2 h, and quenched with acetic acid (0.3 mL, 5 mmol). Solvents were removed in vacuum and the residue was purified by silica gel chromatography (0-10% MeOH/DCM) to afford 1.28 g of compound 197 (93% yield). LRMS (ESI): m/z 470.2 [M+H]⁺, Calcd for C₂₁H₂₇NO₁₁ m/z 470.2.

### Preparation of (2S,3R,4S,5R,6R)-2-(2-((S)-28-(4-((((9H-fluoren-9-yl)methoxy)carbonyl)amino)butyl)-26-oxo-2,5,8,11,14,17,20,23-octaoxa-27-azanonacosan-29-amido)-4-((((4-nitrophenoxy)carbonyl)oxy)methyl)phenoxy)-6-(acetoxymethyl)tetrahydro-2H-pyran-3,4,5-triyl triacetate (198)

To a mixture of carboxylic acid 129 (80 mg, 105 μmol) and compound 197 (40 mg, 85 μmol) in 1 mL of anhydrous DMF were added DIPEA (40 μL) and HATU (40 mg, 105 μmol) at room temperature. Reaction mixture was stirred overnight, concentrated under reduced pressure, and passed through a silica gel column (0-10% MeOH/DCM as eluent). The obtained semi-purified intermediate was dissolved in 1 mL DCM and treated with DIPEA (40 μL) and bis-PNP carbonate (32 mg, 105 μmol) at room temperature. Reaction mixture was stirred overnight and then directly purified by silica gel chromatography (0-10% MeOH/DCM) to afford 50 mg of compound 198 (36 μmol, 42% yield). LRMS (ESI): m/z 1379.5 [M+H]⁺, Calcd for C₆₇H₈₆N₄O₂₇ m/z 1379.6.

### Preparation of 3-((S)-28-(4-aminobutyl)-26-oxo-2,5,8,11,14,17,20,23-octaoxa-27-azanonacosan-29-amido)-4-(((2S,3R,4S,5S,6R)-3,4,5-trihydroxy-6-(hydroxymethyl)tetrahydro-2H-pyran-2-yl)oxy)benzyl (2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamate (199)

To a solution of belotecan 16 (HCl salt, 10 mg, 21 μmol) in 1 mL of anhydrous DMF were added DIPEA (10 μL) and HOAt (1 mg), followed by PNP carbonate 198 (25 mg, 18 mol) at room temperature. Reaction mixture was stirred for 2 days, then concentrated under vacuum. The residue was dissolved in 1 mL of THF, cooled to 0° C., and treated with 1M aqueous LiOH (1 mL). Reaction mixture was allowed to warm up to room temperature, stirred for 3 hours, and then purified by reversed-phase prep HPLC (10-55% CH₃CN—H₂O with 0.05% TFA) to afford 5 mg (4 μmol, 22% yield) of compound 199 as a yellow solid. LRMS (ESI): m/z 1283.6 [M+H]⁺, Calcd for C₆₃H₉₀N₆O₂₂ m/z 1283.6.

### Preparation of 3-((S)-28-(4-(3-(2-((1,2-dimethylhydrazinyl)methyl)-5-((S)-28-((5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)-2-(((2S,3R,4S,5S,6R)-3,4,5-trihydroxy-6-(hydroxymethyl)tetrahydro-2H-pyran-2-yl)oxy)phenyl)carbamoyl)-26,34-dioxo-2,5,8,11,14,17,20,23-octaoxa-27,33-diazaheptatriacontan-37-amido)-1H-indol-1-yl)propanamido)butyl)-26-oxo-2,5,8,11,14,17,20,23-octaoxa-27-azanonacosan-29-amido)-4-(((2S,3R,4S,5S,6R)-3,4,5-trihydroxy-6-(hydroxymethyl)tetrahydro-2H-pyran-2-yl)oxy)benzyl (2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamate (200)

To a solution of compound 199 (8 mg, 6 μmol) in 1 mL of anhydrous DMF were added DIPEA (6 μL) and HOAt (1 mg), followed by bis-PFP ester 11 (3 mg, 3 μmol) in portions over 30 minutes at room temperature. After 1 hour, piperidine (100 μL) was added directly to the reaction mixture. After 20 minutes, reaction mixture was purified by reversed phase prep HPLC (10-65% CH₃CN—H₂O with 0.05% TFA). Pure fractions were pooled and lyophilized to give 2 mg (0.7 μmol, 23% yield) of compound 200 as a yellowish solid. LRMS (ESI): m/z 1454.2 [M+2H]²⁺, Calcd for C₁₄₄H₂₀₀N₁₆O₄₇ m/z 1454.2.

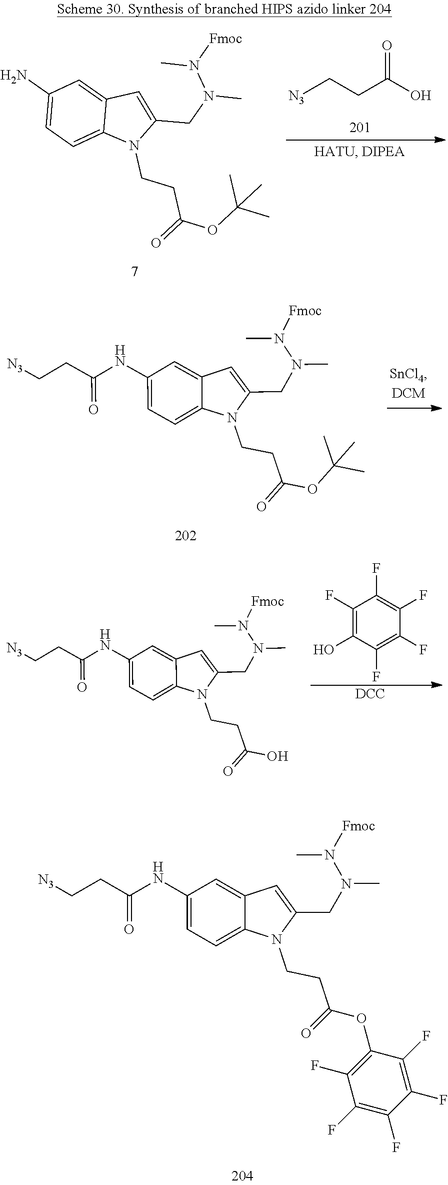

### Preparation of (9H-fluoren-9-yl)methyl 2-((5-(3-azidopropanamido)-1-(3-(tert-butoxy)-3-oxopropyl)-1H-indol-2-yl)methyl)-1,2-dimethylhydrazine-1-carboxylate (202)

To a mixture of compound 7 (200 mg, 0.36 mmol) and azide 201 (42 mg, 0.36 mmol) in 2 mL of anhydrous DMF were added HATU (137 mg, 0.36 mmol) and DIPEA (0.19 mL) at room temperature. The resulting mixture was stirred for 2 h until reaction was judged complete by HPLC analysis. The mixture was purified by reversed-phase chromatography (0-70% CH₃CN—H₂O with 0.05% TFA). Solvents were removed in vacuum to afford compound 202 as a brown oil (210 mg, 0.32 mmol, 89% yield). LRMS (ESI): m/z 652.3 [M+H]⁺, Calcd for C₃₆H₄₁N₇O₅ m/z 652.3.

### Preparation of 3-(2-((2-(((9H fluoren-9-yl)methoxy)carbonyl)-1,2-dimethylhydrazineyl)methyl)-5-(3-azidopropanamido)-1H-indol-1-yl)propanoic acid (203)

To a solution of compound 202 (210 mg, 0.32 mmol) in 1 mL of anhydrous DCM were added solution of tin(IV) chloride (1 mL, 1M in DCM) at 0° C. Reaction mixture was allowed to warm up to room temperature, stirred for 30 minutes, and then quenched with water (5 mL) and extracted with ethyl acetate. Organic layer was dried over sodium sulfate, solvents removed in vacuum to give 180 mg of compound 203 (0.30 mmol, 94% yield) as a brown oil, which was used further without purification. LRMS (ESI): m/z 596.3 [M+H]⁺, Calcd for C₃₂H₃₃N₇O₅ m/z 596.3.

### Preparation of (9H-fluoren-9-yl)methyl 2-((5-(3-azidopropanamido)-1-(3-oxo-3-(perfluorophenoxy)propyl)-1H-indol-2-yl)methyl)-1,2-dimethylhydrazine-1-carboxylate (204)

To a mixture of carboxylic acid 203 (180 mg, 0.30 mmol) and pentafluorophenol (555 mg, 3 mmol) in 4 mL of anhydrous THF were added DCC (622 mg, 3 mmol) at room temperature. Reaction mixture was stirred overnight, filtered, and concentrated under reduced pressure. The residue was purified by silica gel chromatography (0-40% EtOAc-hexane) to give 150 mg of compound 204 as a brownish solid (0.20 mmol, 67% yield). LRMS (ESI): m/z 762.2 [M+H]⁺, Calcd for C₃₈H₃₂F₅N₇O₅ m/z 762.2.

### Example 2

### Synthesis of Dual-Payload Constructs Utilizing Branched Orthogonal HIPS Linker 204

By having two orthogonal reactive groups (i.e. activated ester and azide), the HIPS linker 204 serves as a modular scaffold to construct a variety of dual-payload drug-linkers for ADCs. This approach allows ADCs to include various chemical entities ranging from biologically active small molecule payloads (cytotoxins, kinase inhibitors, immuno-stimulants, etc.) to more molecularly complex sub-units such as peptides, oligonucleotides, fragment antibodies, etc. Thus, for example, therapeutically relevant iRGD peptide 206 is effectively combined with Topoisomerase I inhibitor (belotecan) using branched linker 204 as shown in Scheme 31. First, the activated ester in 204 reacts with the belotecan linker 130 to form intermediate 205. Separately, N-terminus of protected iRGD peptide is further functionalized to attach a PEG spacer and alkyne moiety and to furnish subunit 209. Finally, copper-catalyzed azide-alkyne cycloaddition (CuAAC) is used to create a stable permanent 1,2,3-triazole linkage between the two large subunits 205 and 209. Activation of the HIPS moiety by Fmoc-deprotection produces construct 210, which upon conjugation with an antibody produces a dual-payload conjugate containing both iRGD peptide and a small-molecule inhibitor (belotecan). Using the same approach, iRGD/MMAE construct 211 is prepared.

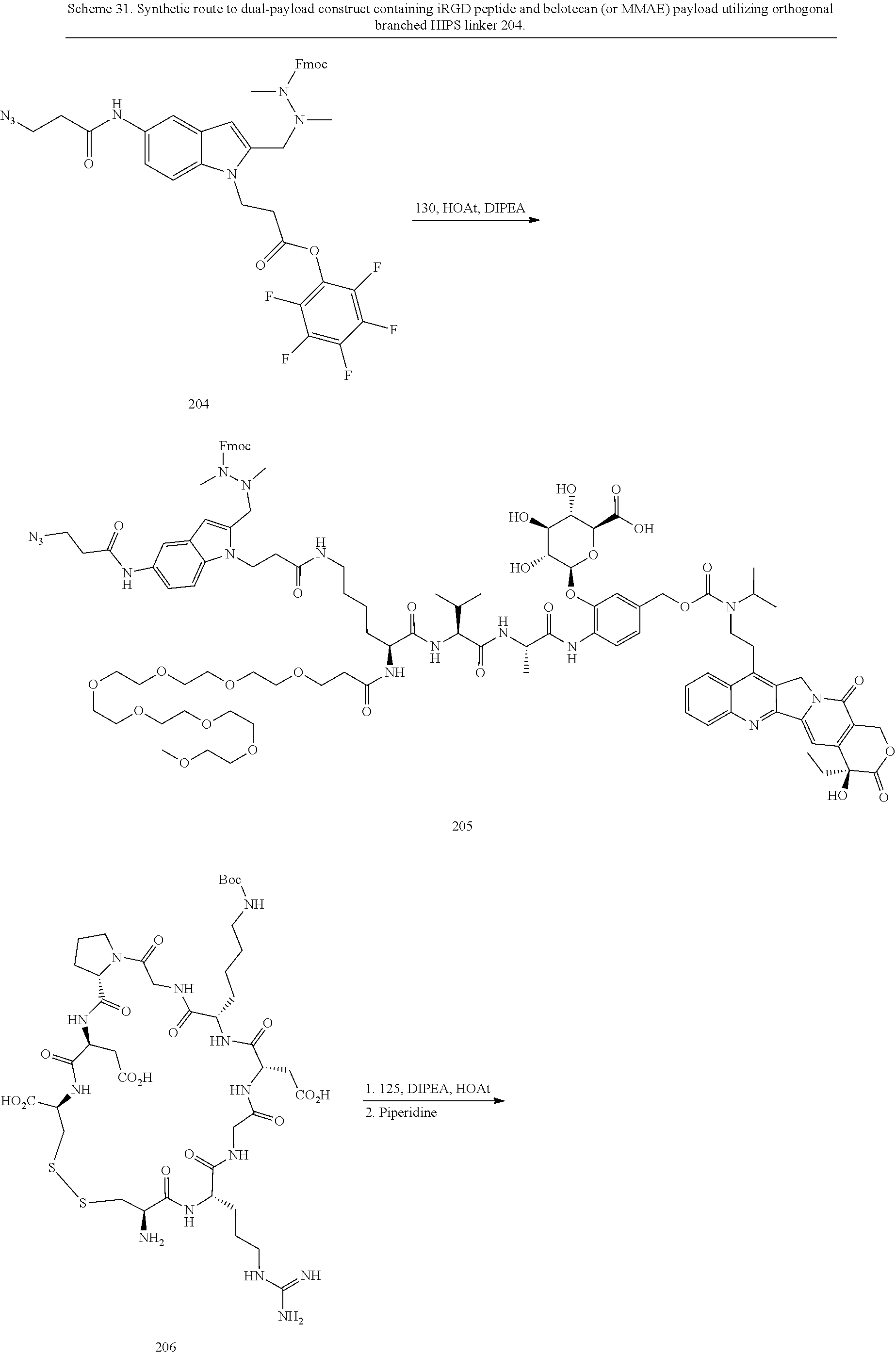

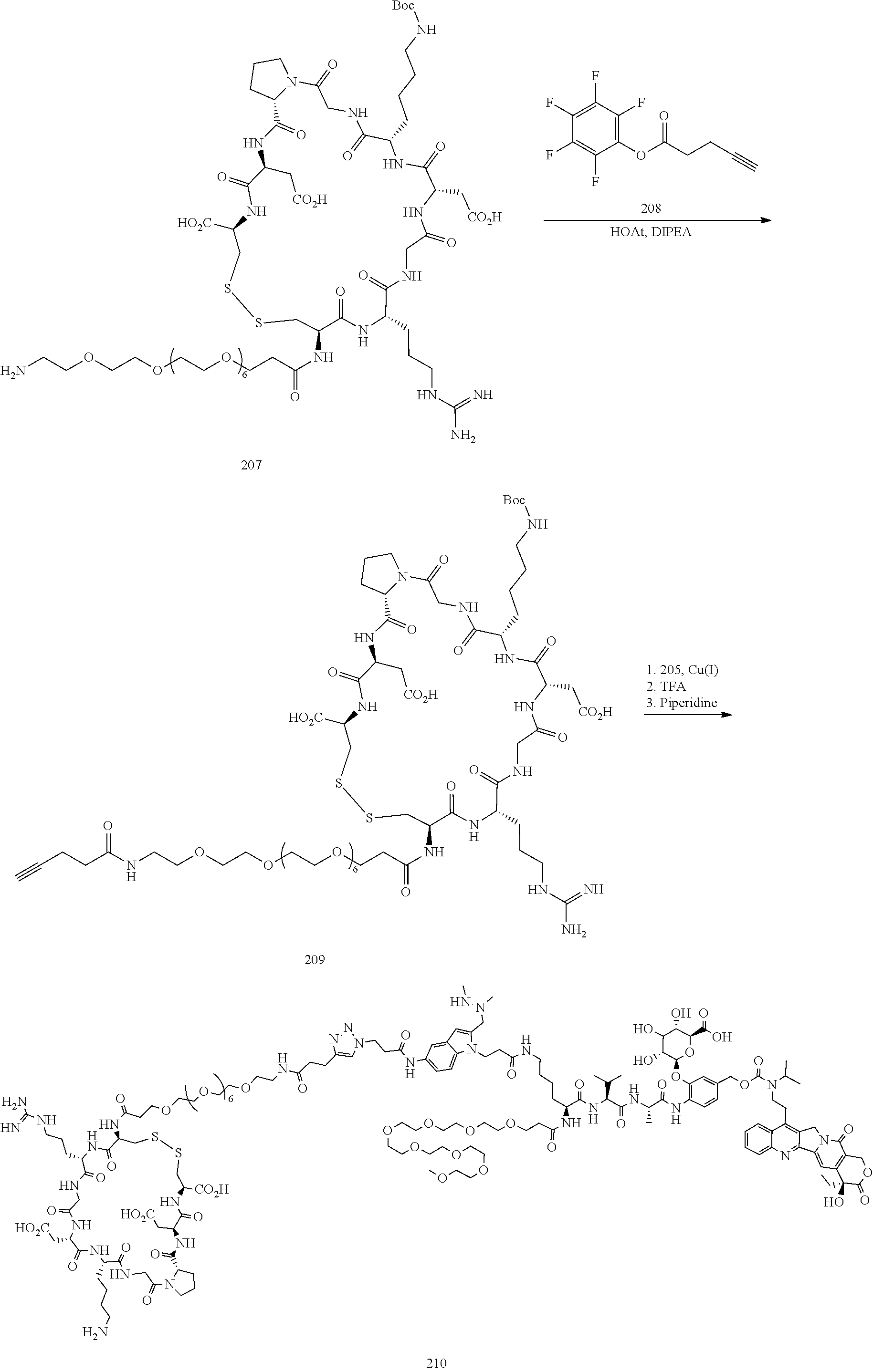

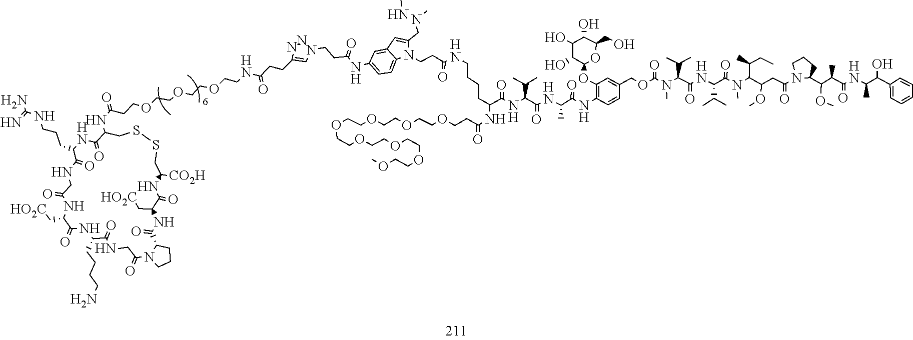

### Example 3

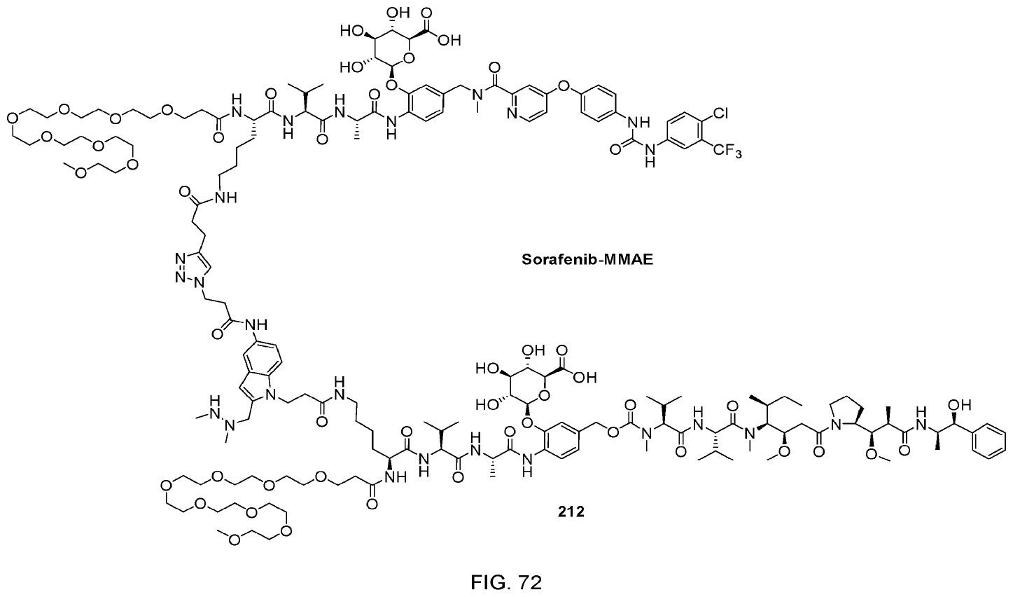

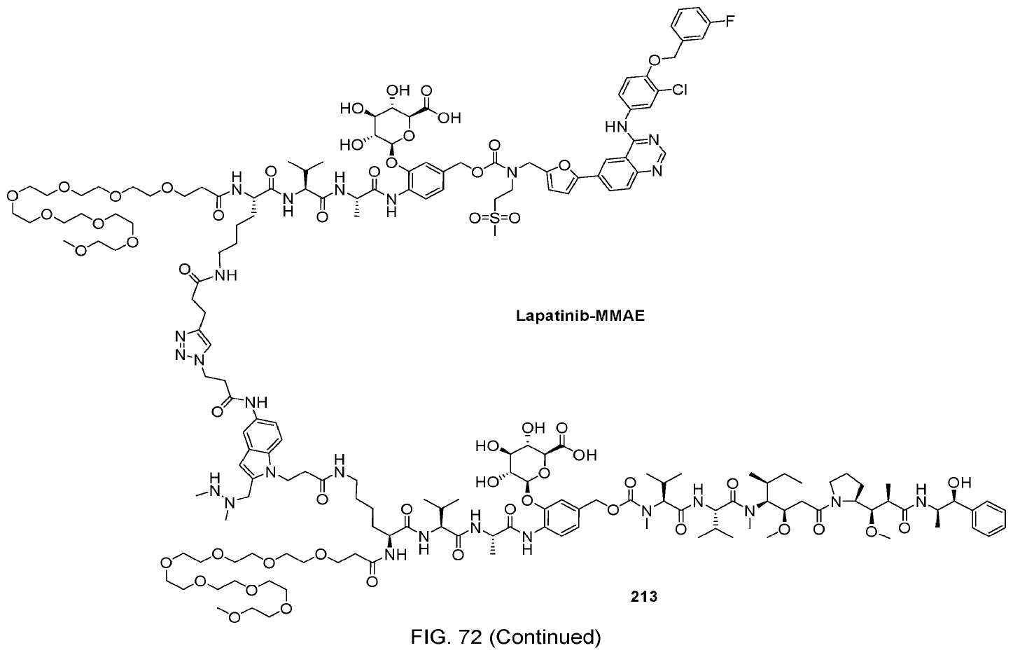

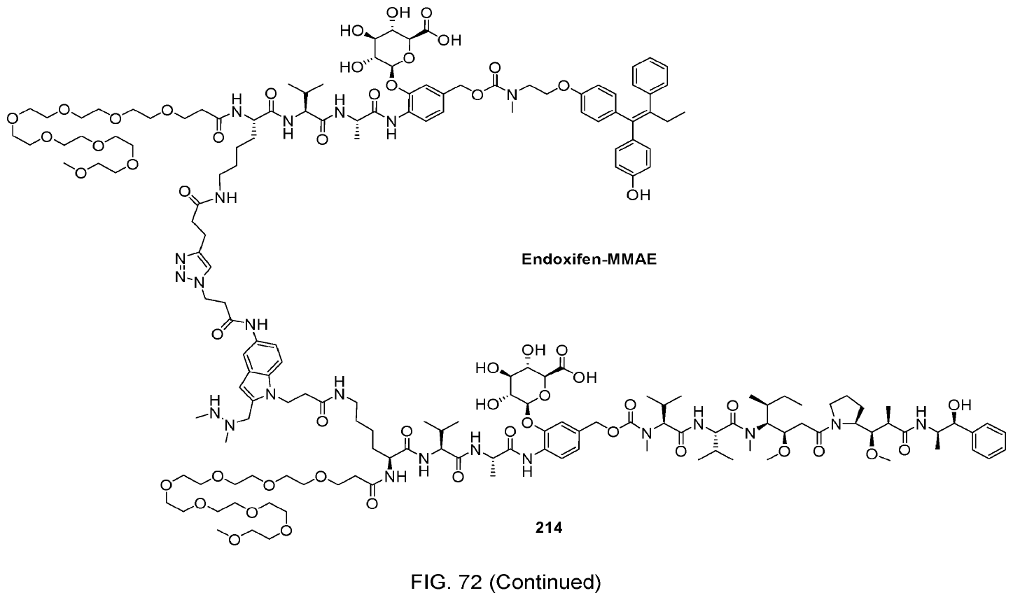

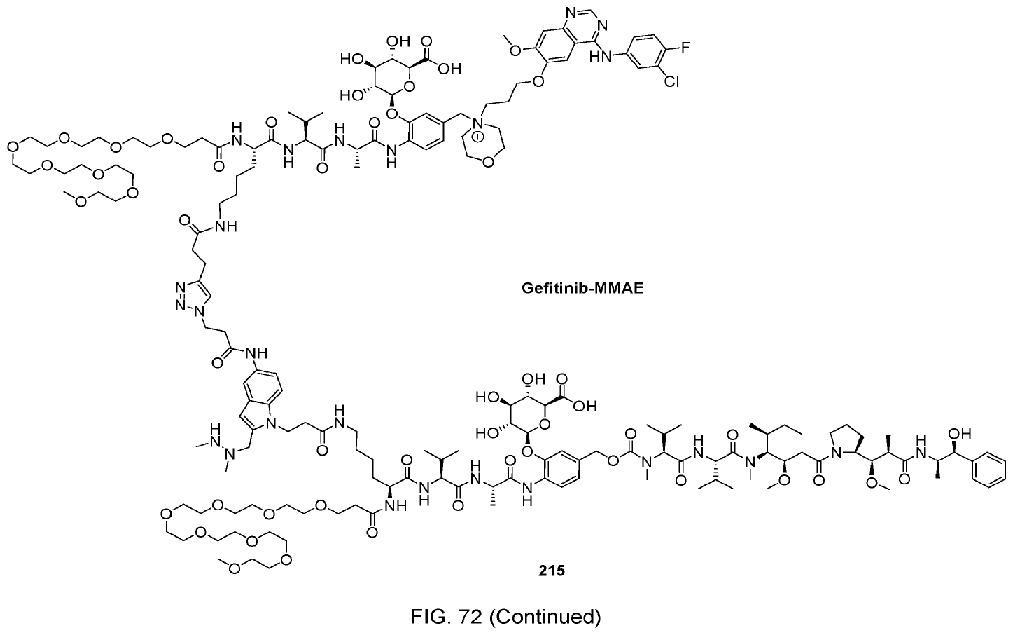

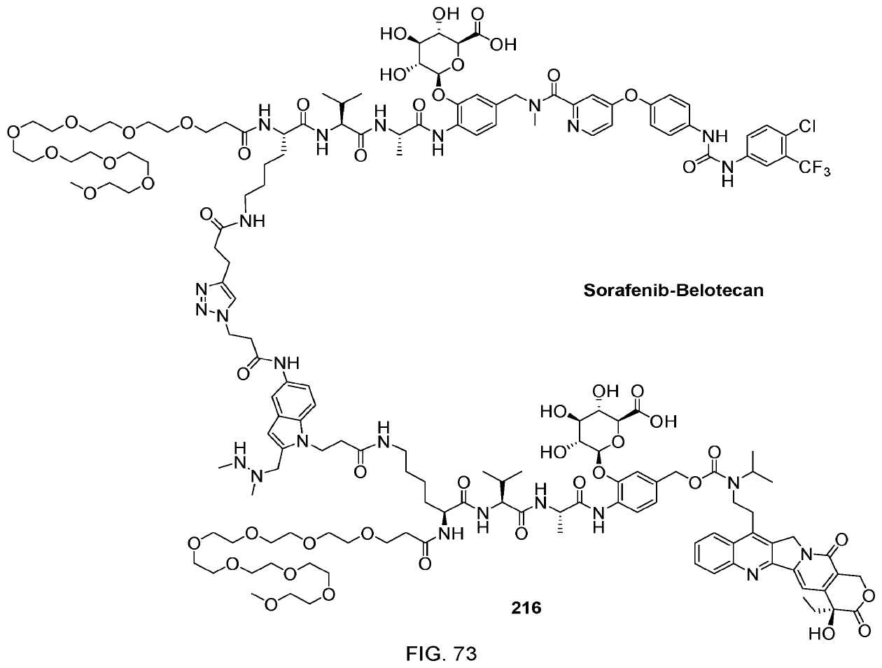

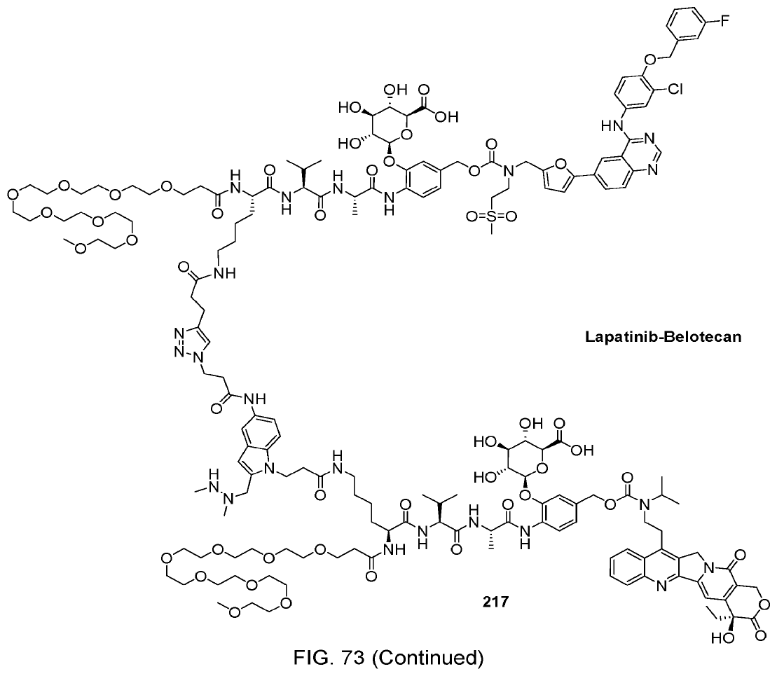

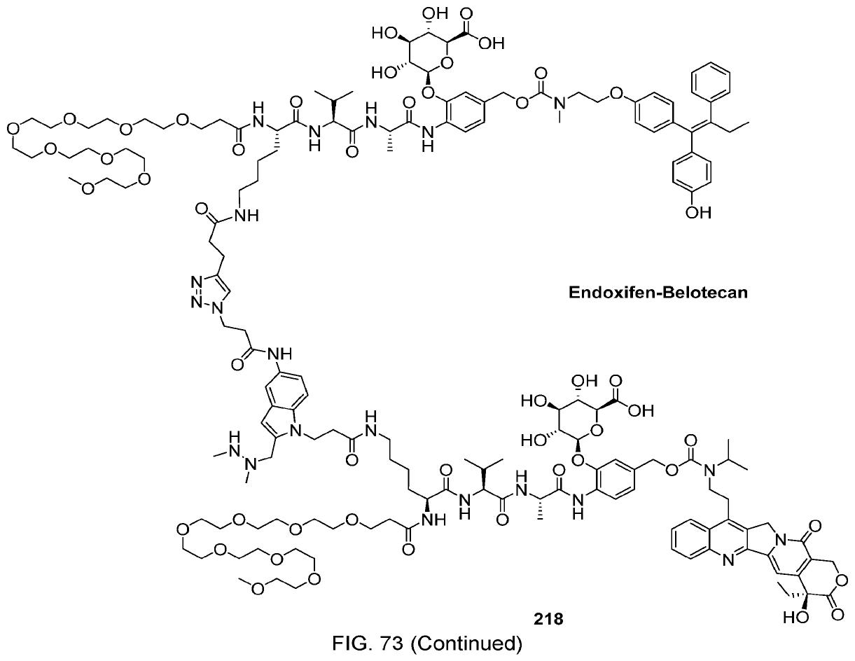

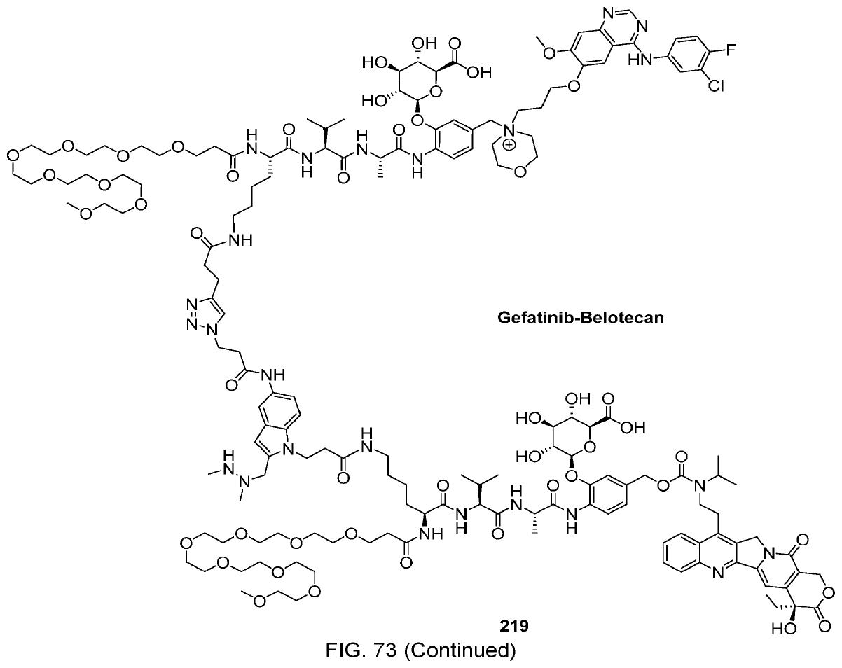

Similar to Example 2 described above, branched HIPS linker 204 is used to build dual-payload constructs which combine two different payloads, each with a different mechanism of action (MOA). ADCs prepared using such dual-payload constructs may offer superior therapeutic activity compared to ADCs carrying the same payload due to synergistic effects of their coaction within the same tumor microenvironment. Examples of such dual payload constructs are summarized in FIG. 72 and FIG. 73.

### Example 4

### Synthesis and Use of Alternative Branched Bis-HIPS Linkers

Branched HIPS linker bis-PFP ester 11 shown in Scheme 1 provided quick access to constructs carrying the same payload at positions 1 and 5 of the indole ring. Using alternative bis-HIPS linkers carrying payloads at different relative positions with respect to the indole ring, provides additional opportunities to finely tune the drug-linker construct architecture, improving conjugation efficiency as well as the biophysical properties of the resulting antibody-drug conjugate. A synthetic approach towards such alternative branched linkers is shown in Scheme 32. Double-alkylation of 6-oxy NH-indole 222 with bromoacetate gives two modifiable chemical handles at positions 1 and 6. Installation of the dimethylhydrazine moiety, removal of protecting groups, and bis-PFP-esterification affords branched linker 227. A similar synthetic route leads to the 7-oxy-derivative 228.

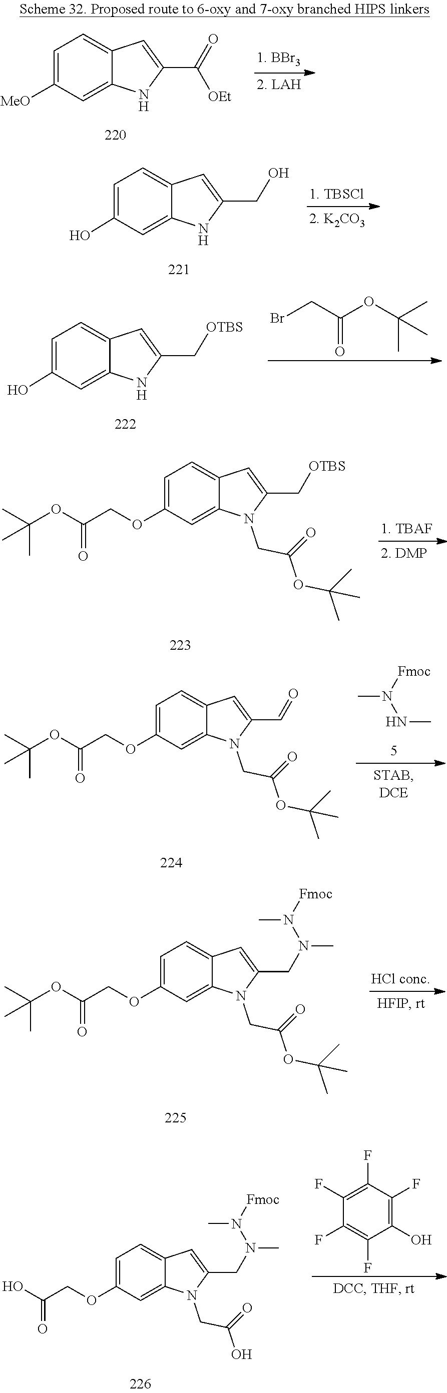

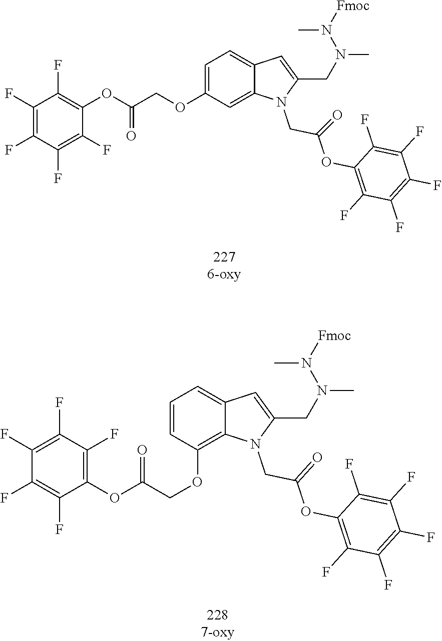

### Example 5

### Bioconjugation, Purification, and HPLC Analytics

Methods: Antibodies (15 mg/mL) bearing one or two aldehyde tags (single or double-tagged constructs) were conjugated to linker-payloads at 1.1 or 1.7 mM, respectively. Reactions proceeded for 72 h at 37° C. in 20 mM sodium citrate, 50 mM NaCl pH 5.5 (20/50 buffer) containing 0.85-2.5% DMA. In some cases, Triton-X-100 was added to 0.25% to improve linker-payload solubility. After conjugation, free drug was removed using a 30 kD MWCO 0.5 mL Amicon spin concentrator. Samples were added to the spin concentrator, centrifuged at 15,000×g for 7 min, then diluted with 450 μL 20 mM sodium citrate, 50 mM NaCl pH 5.5 and centrifuged again. The process was repeated 10 times. To determine the DAR of the final product, ADCs were examined by analytical chromatography using HIC (Tosoh #14947) or PLRP-RP (Agilent PL1912-1802 1000 A, 8 um, 50×2.1 mm) columns. HIC analysis used mobile phase A: 1.5 M ammonium sulfate, 25 mM sodium phosphate pH 7.0, and mobile phase B: 25% isopropanol, 18.75 mM sodium phosphate pH 7.0. PLRP analysis used mobile phase A: 0.1% trifluoroacetic acid in water, and mobile phase B: 0.1% trifluoroacetic acid in acetonitrile. Prior to PLRP analysis, sample was denatured with the addition of 50 mM DTT, 4 M guanidine HCl (final concentrations) and heating at 37° C. for 30 min. To determine aggregation, samples were analyzed using analytical size exclusion chromatography (SEC; Tosoh #08541) with a mobile phase of 300 mM NaCl, 25 mM sodium phosphate pH 6.8 with 5% isopropanol.

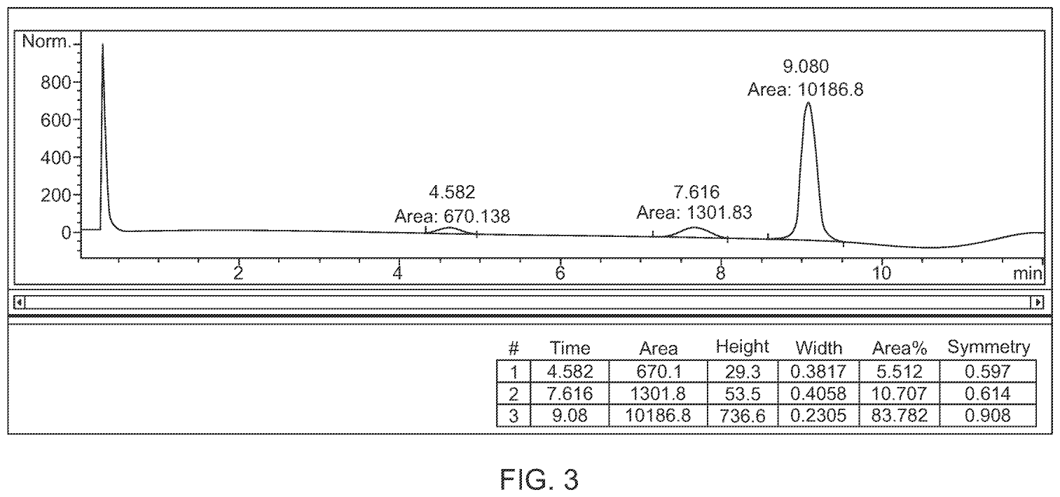

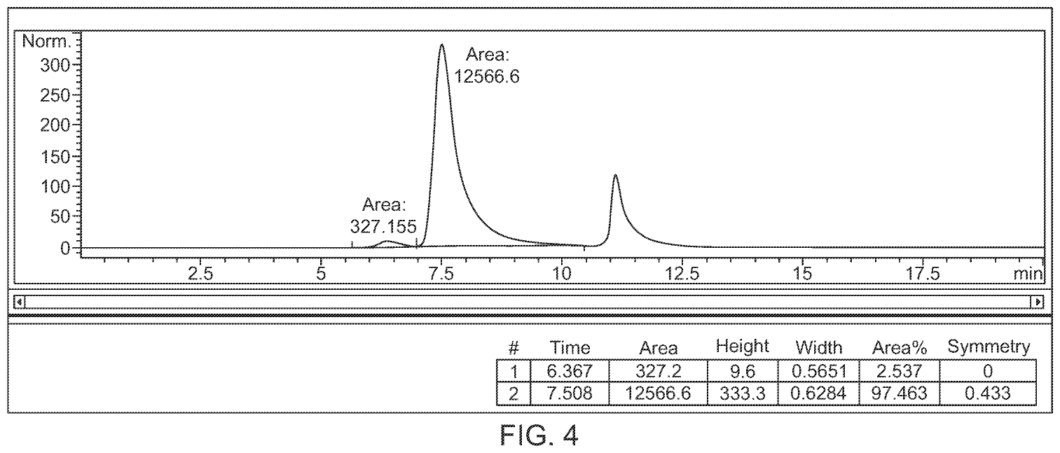

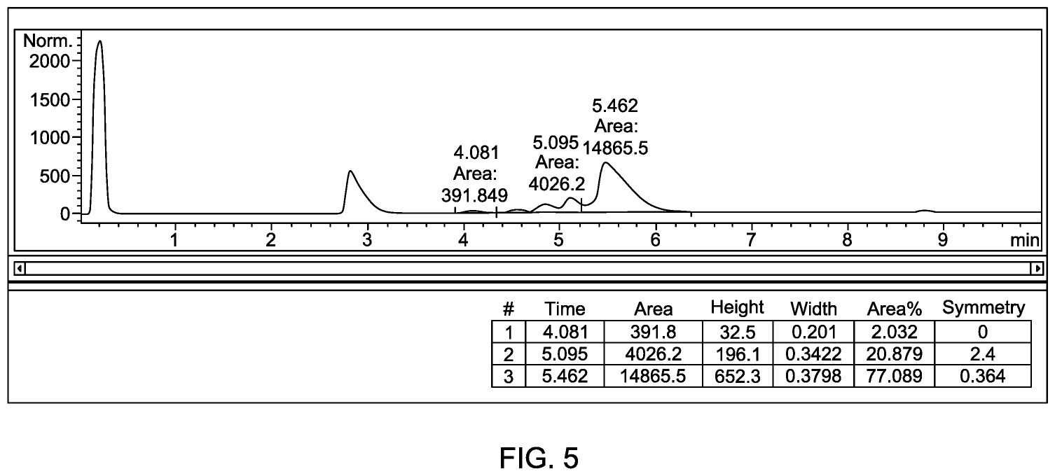

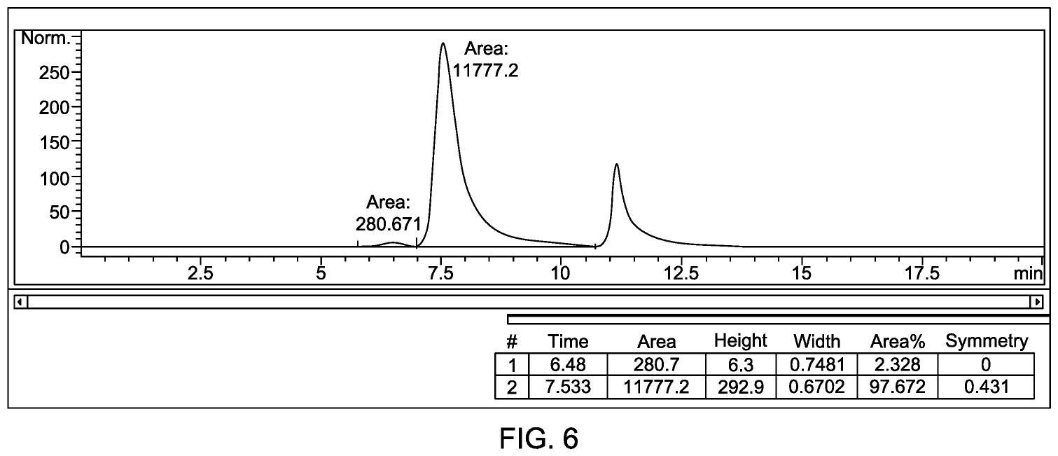

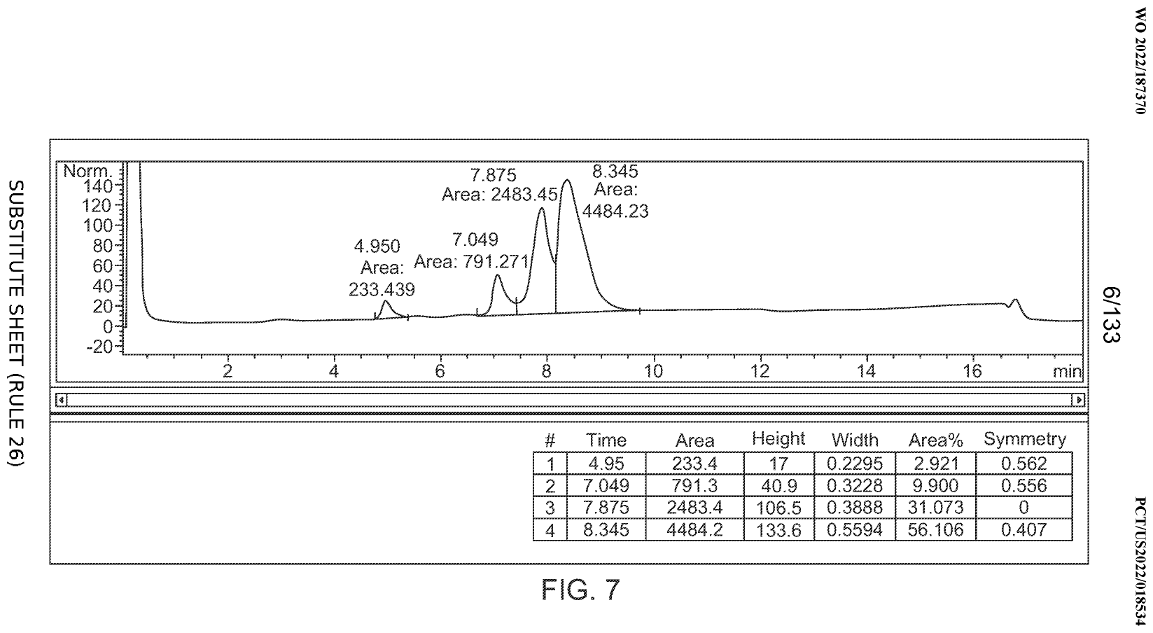

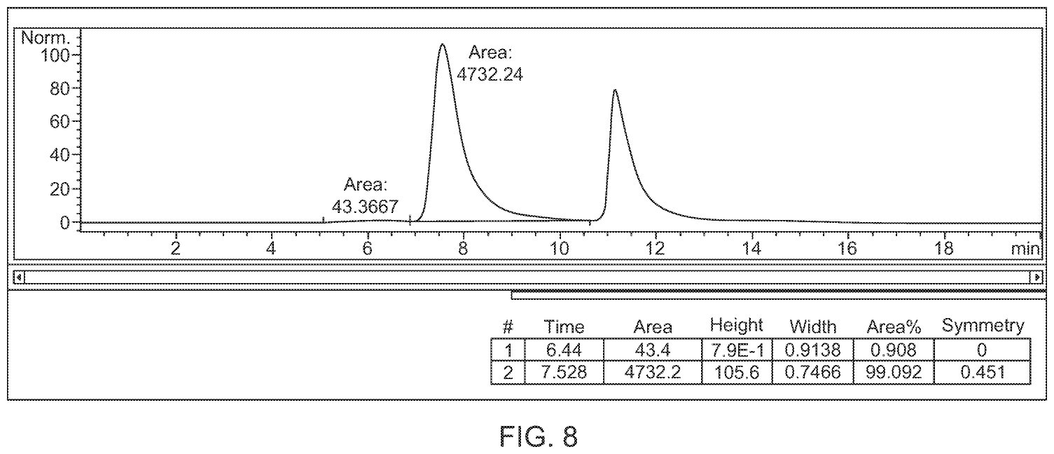

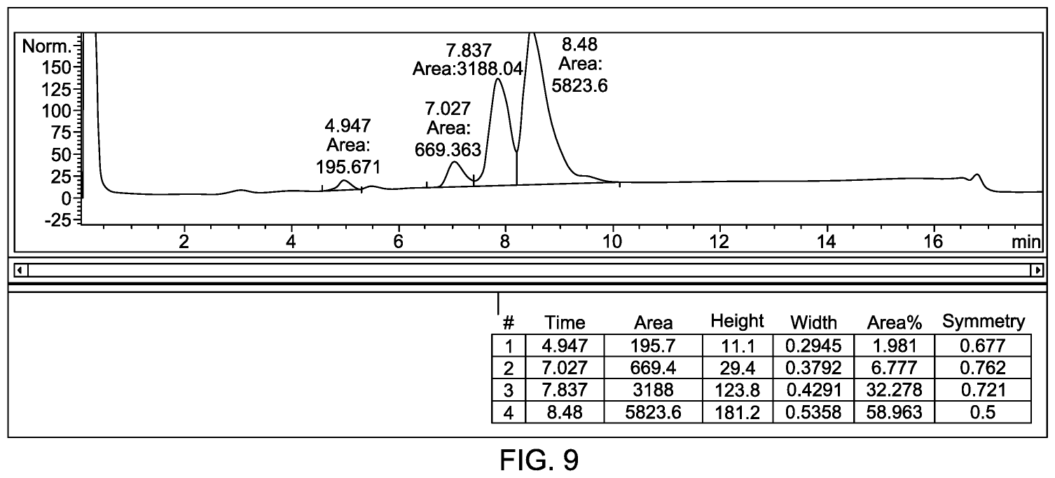

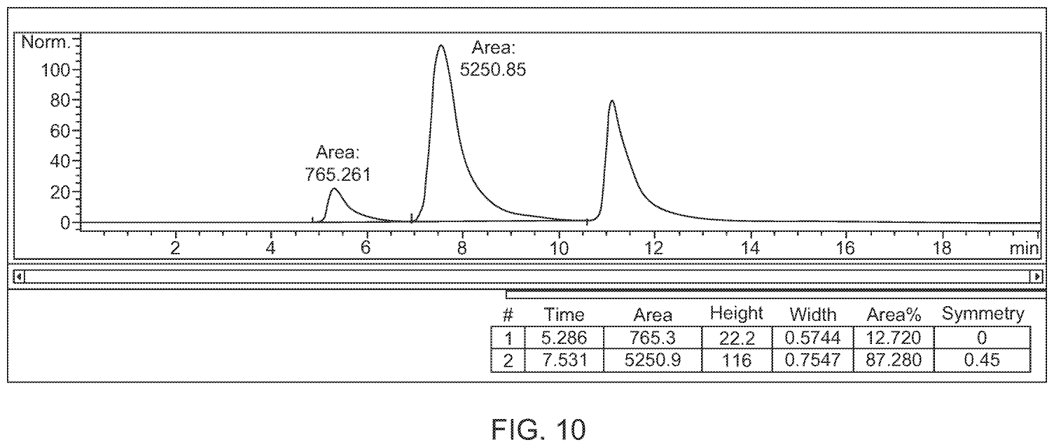

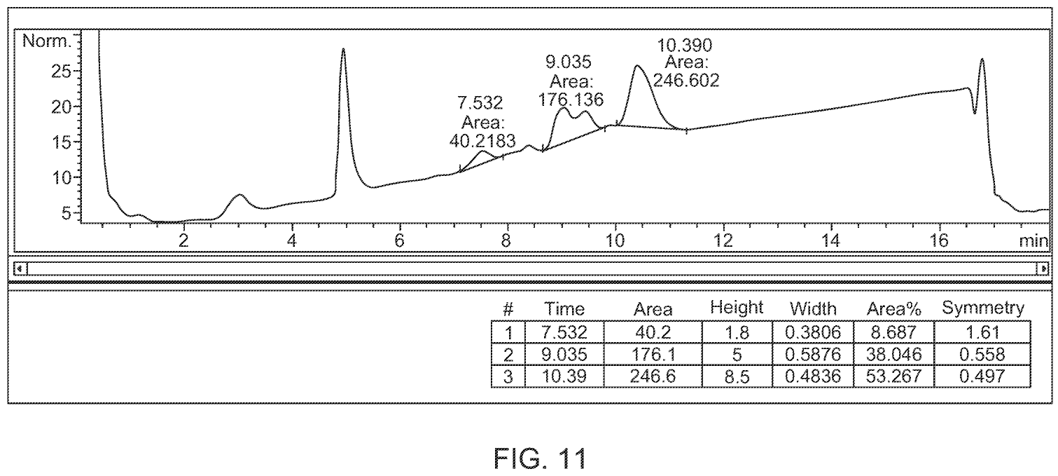

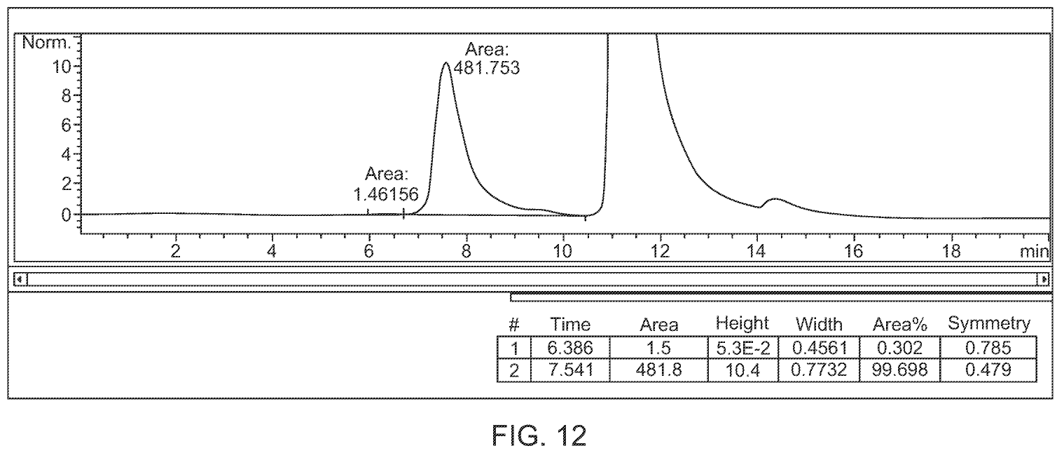

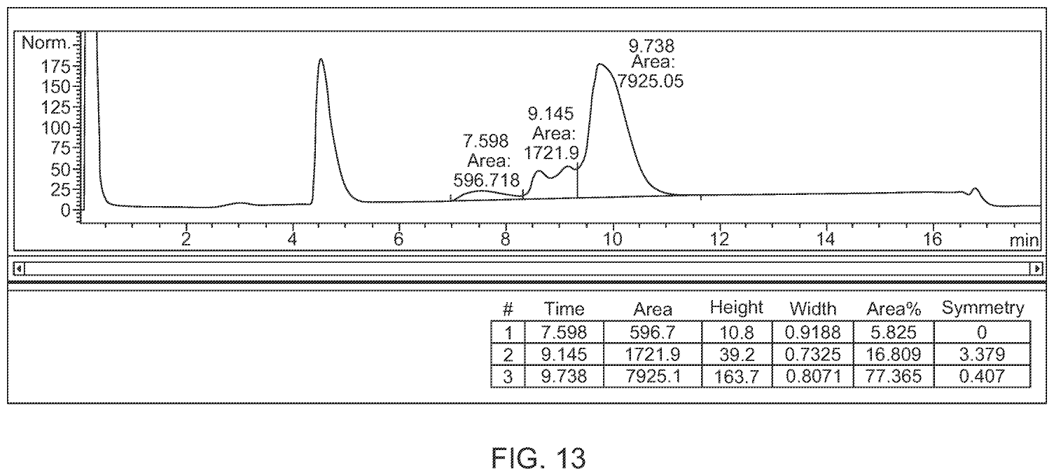

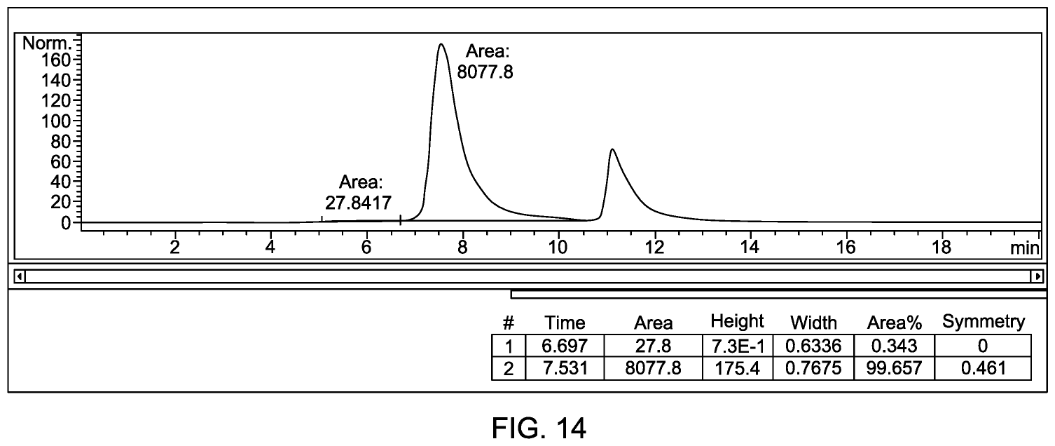

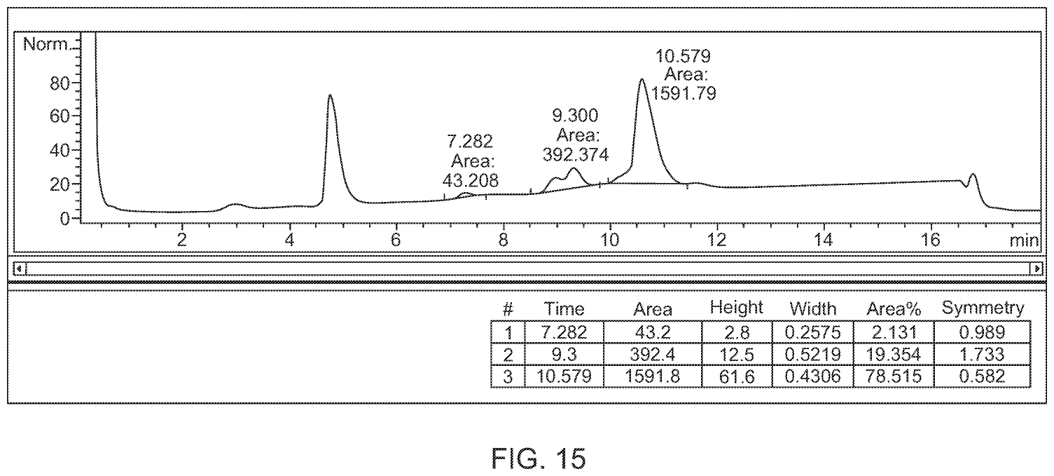

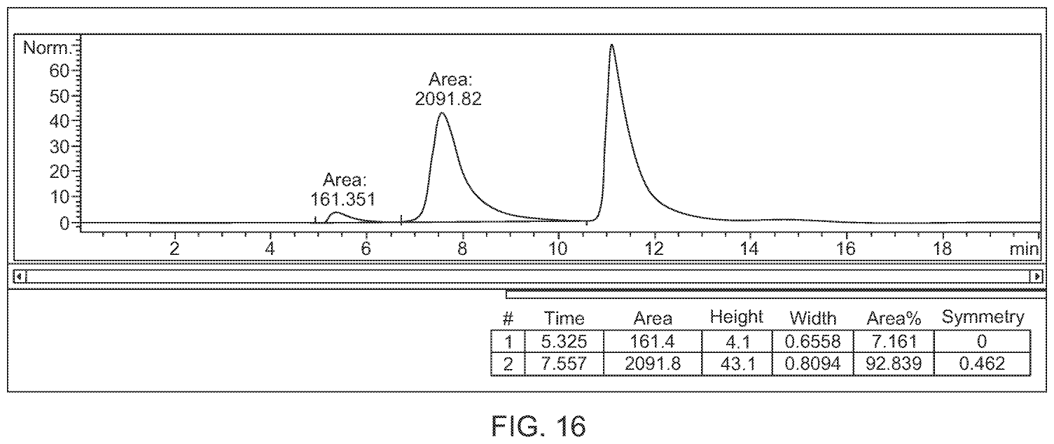

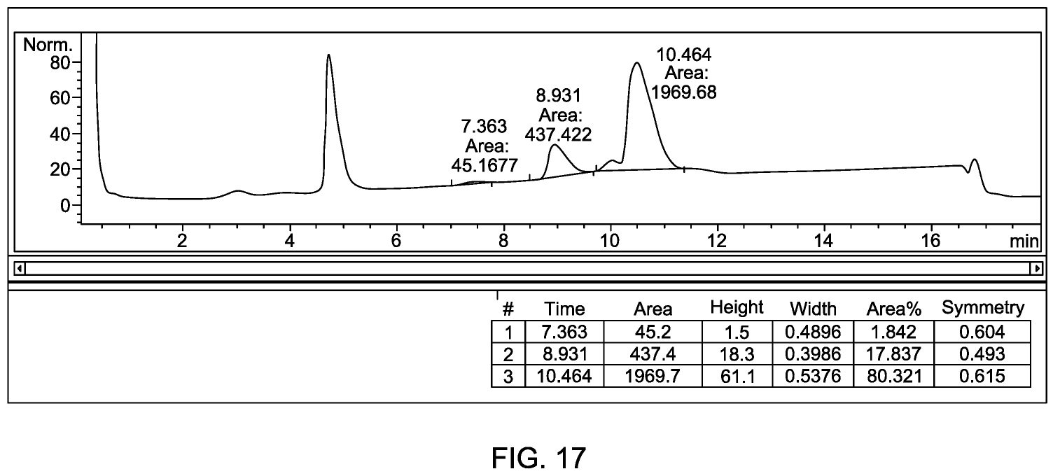

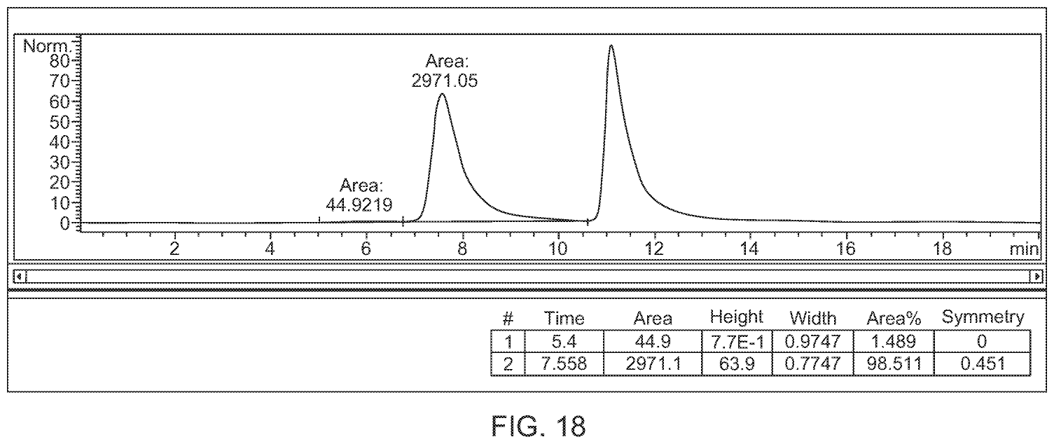

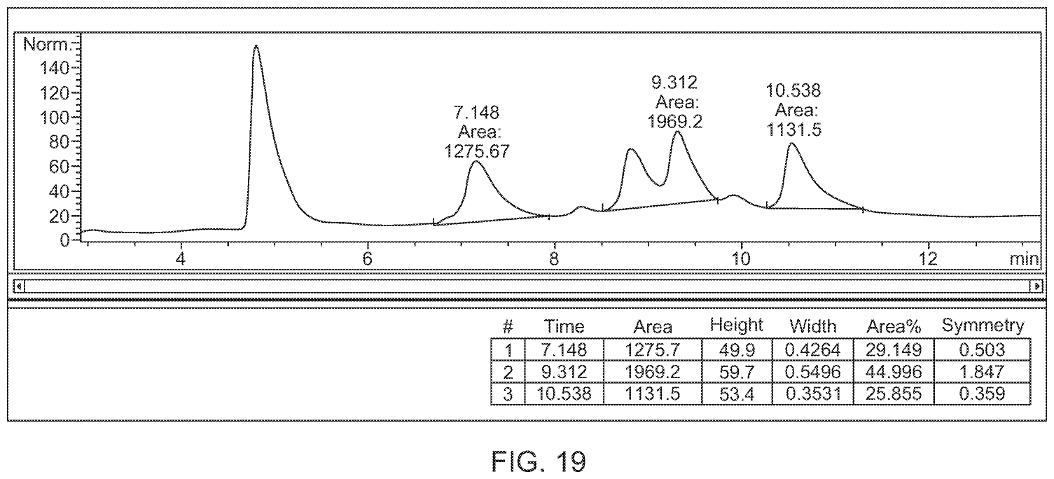

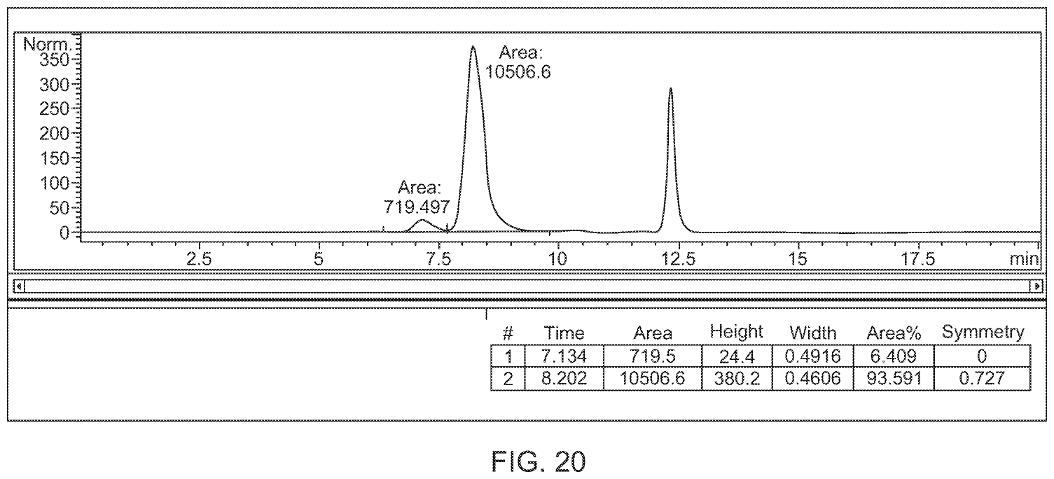

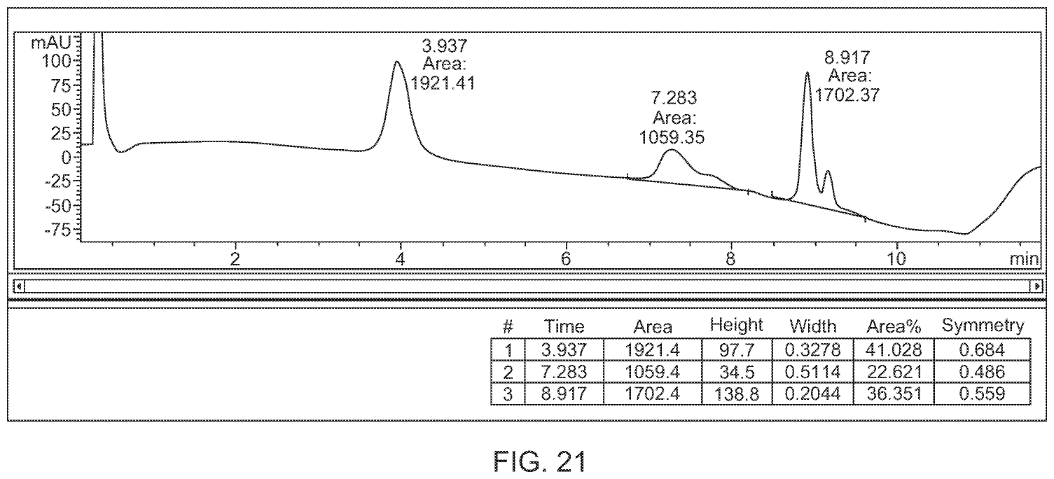

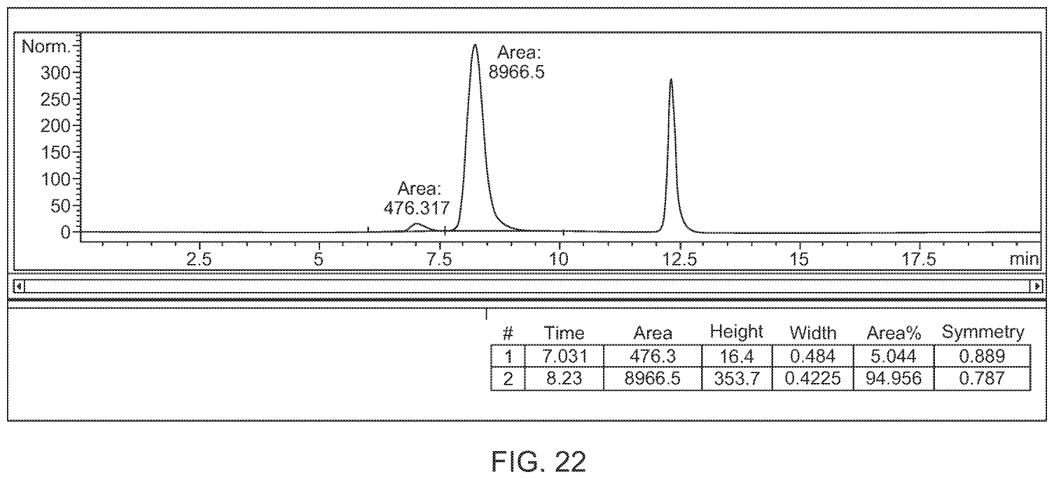

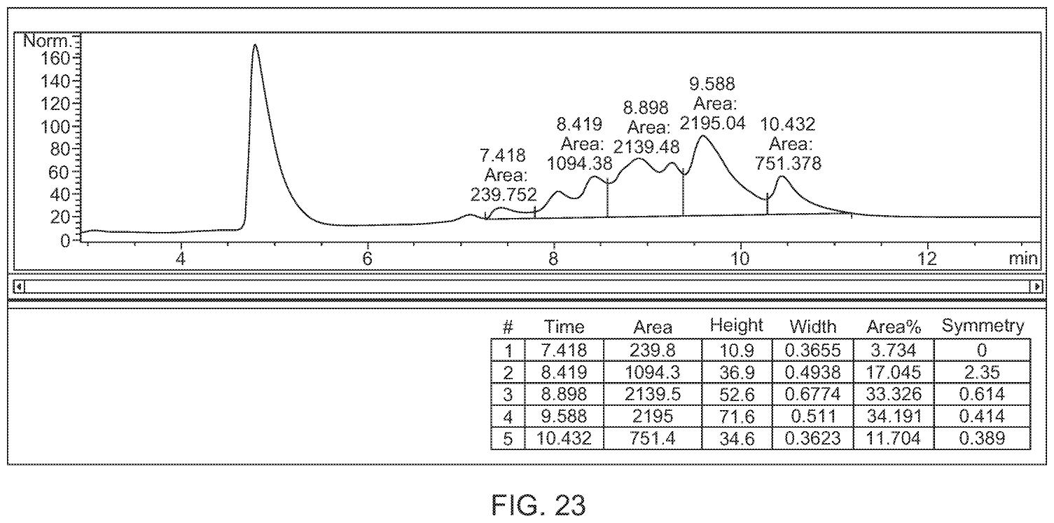

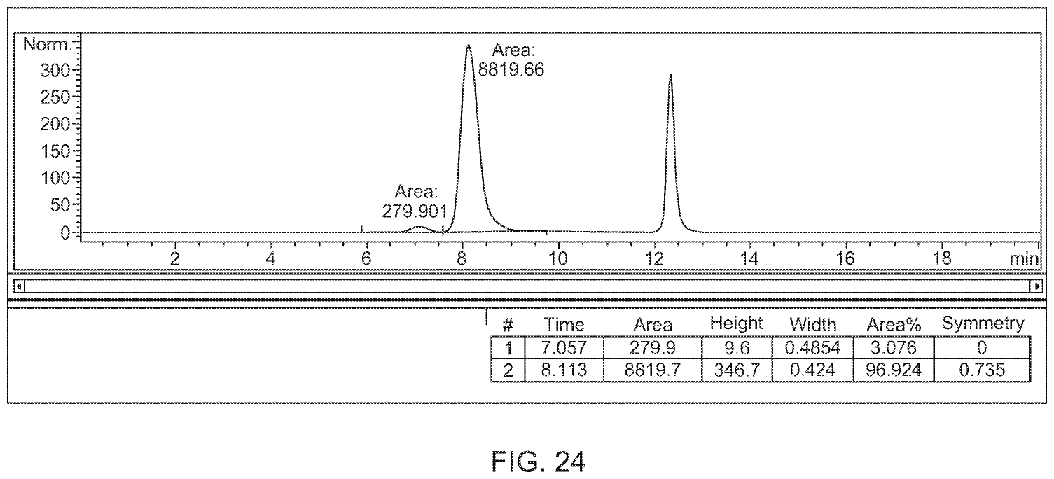

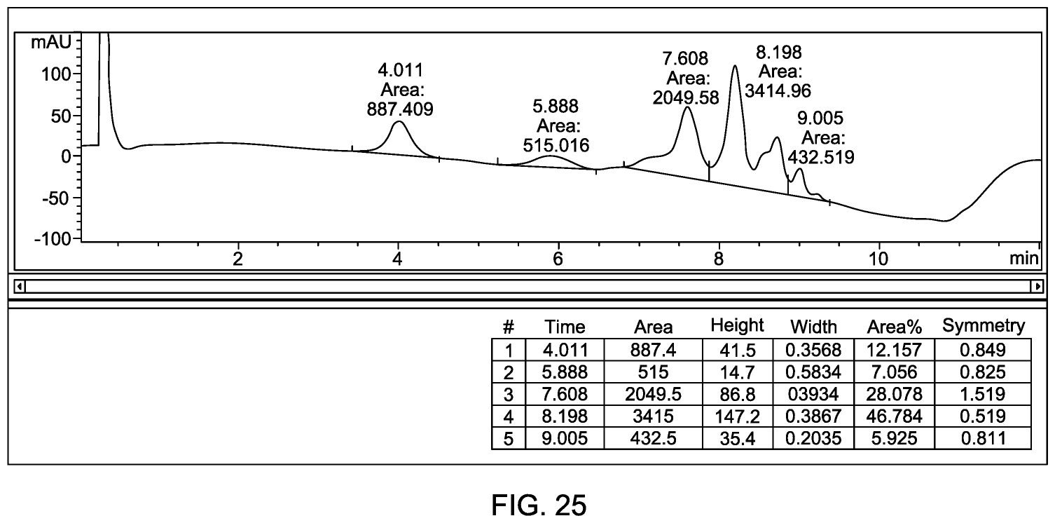

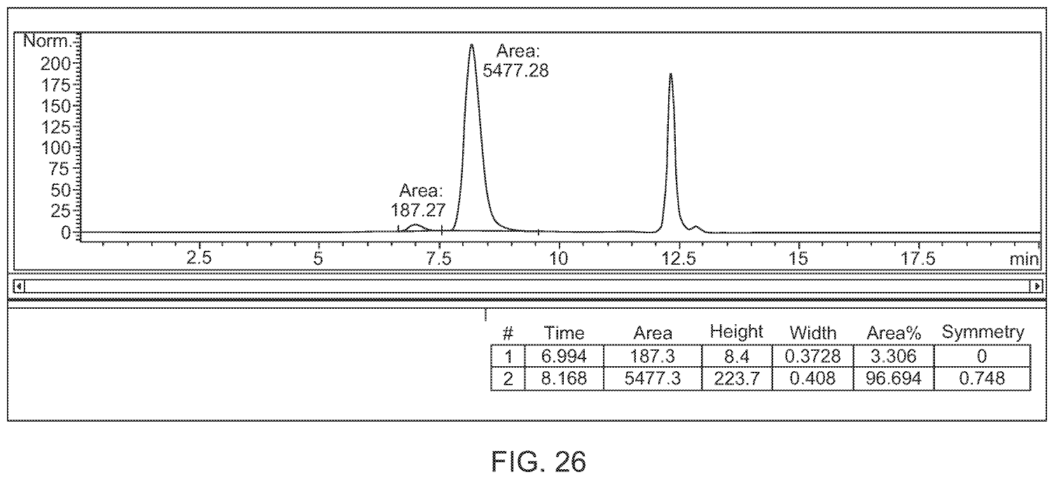

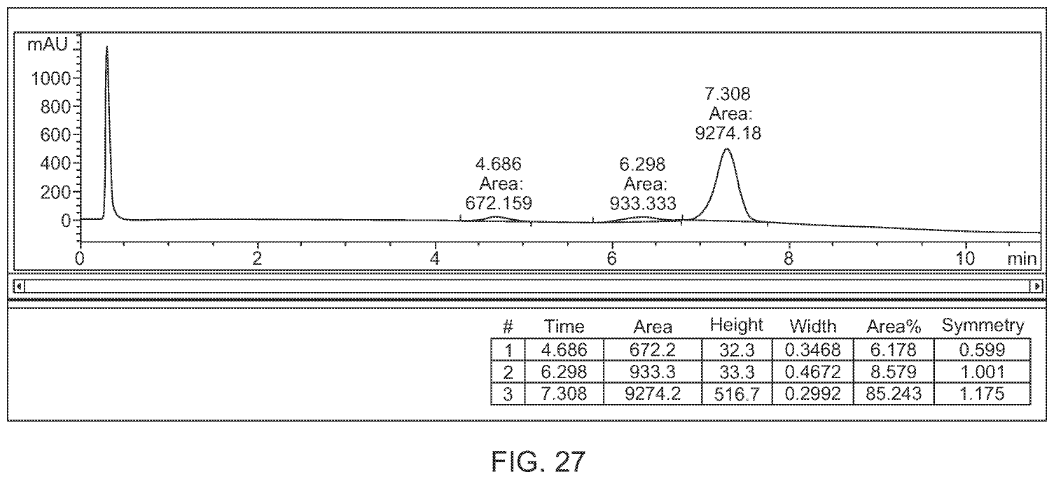

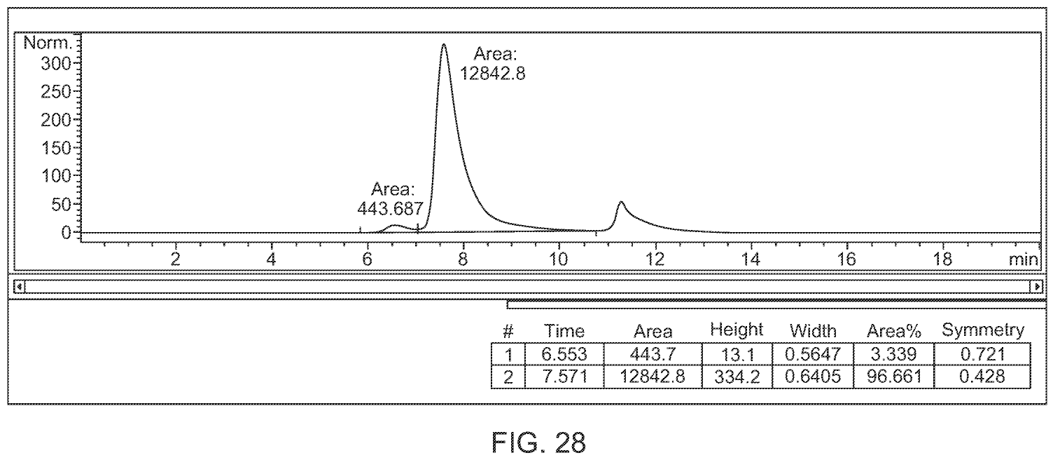

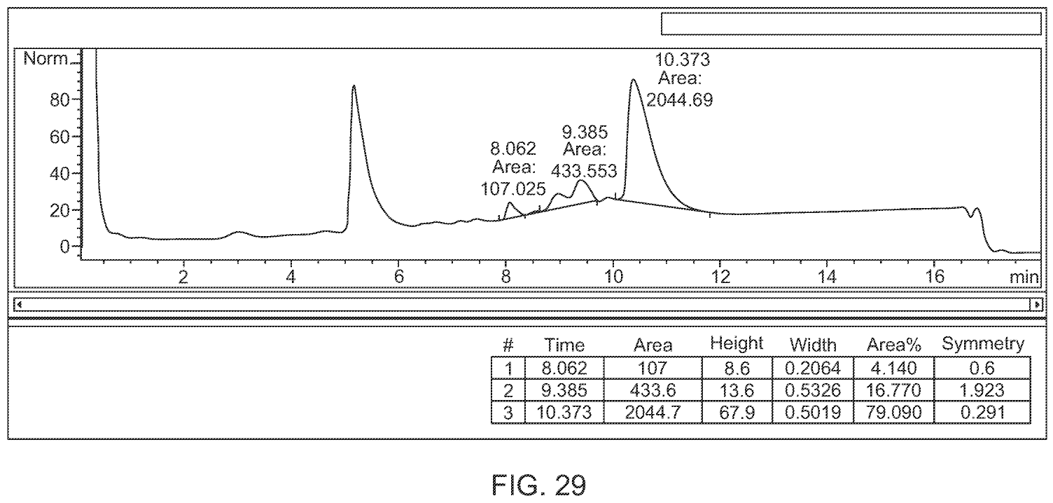

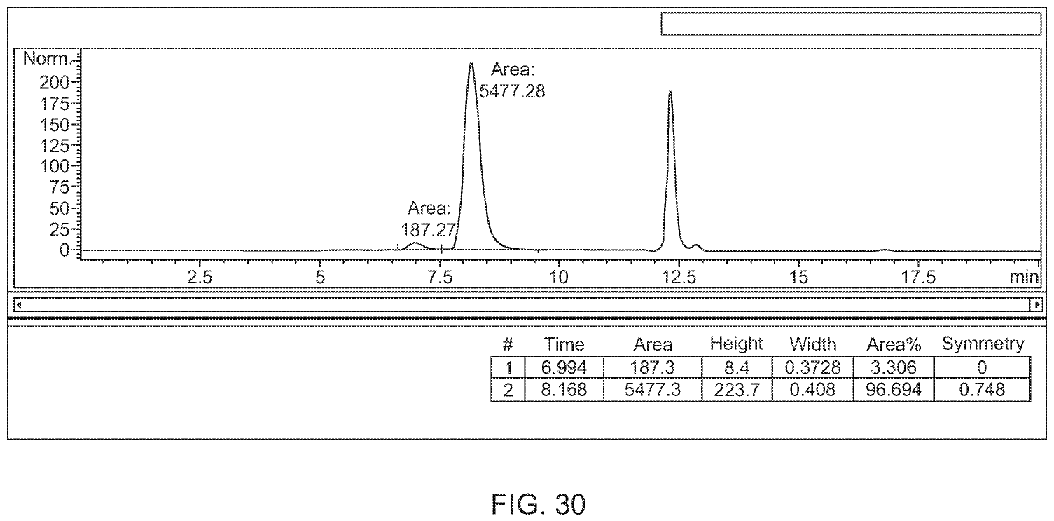

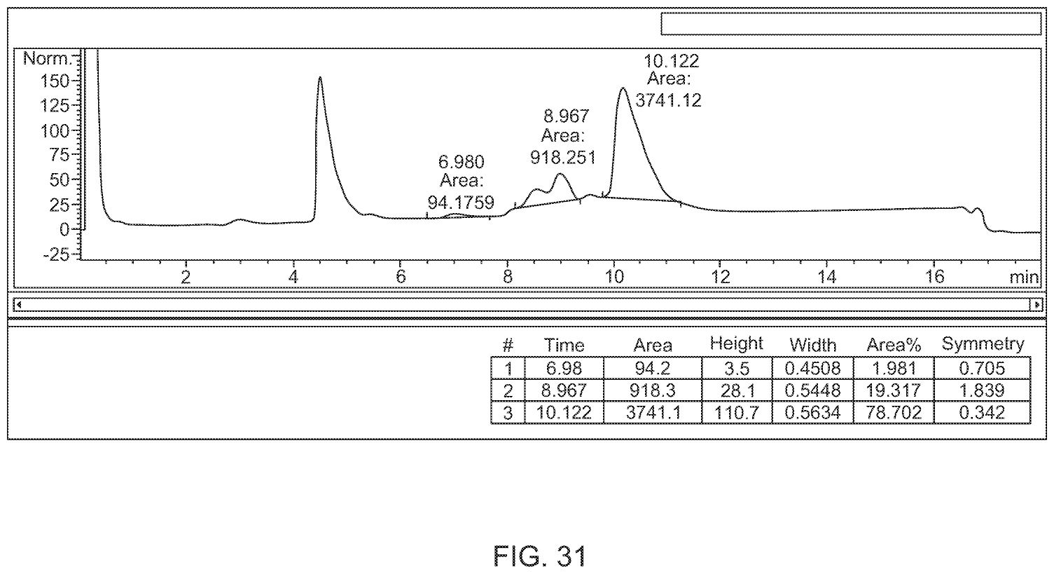

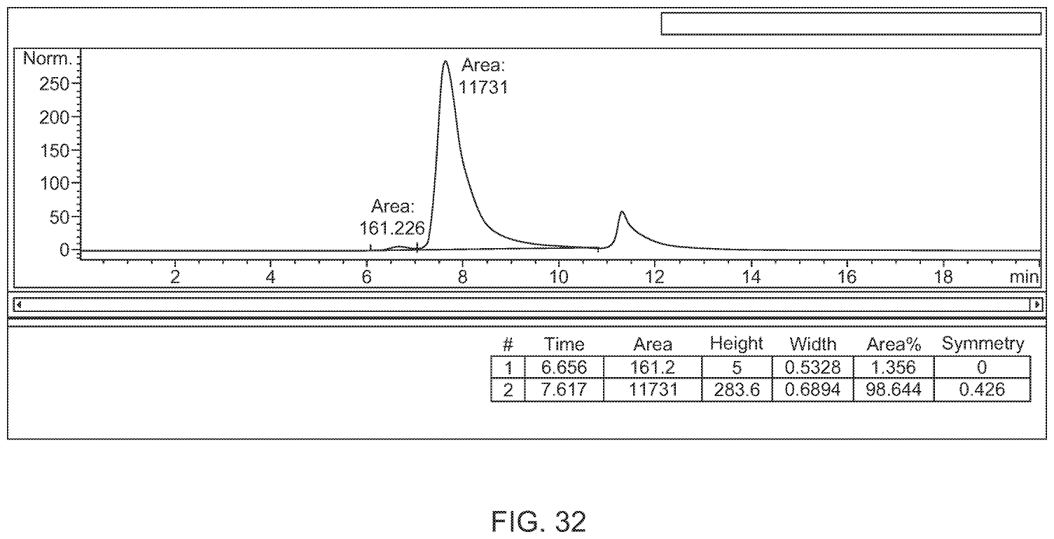

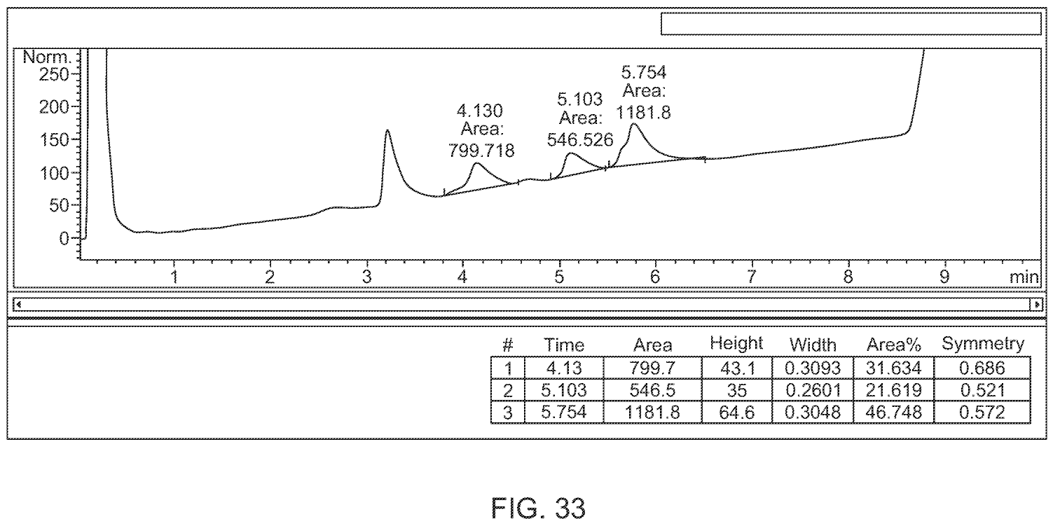

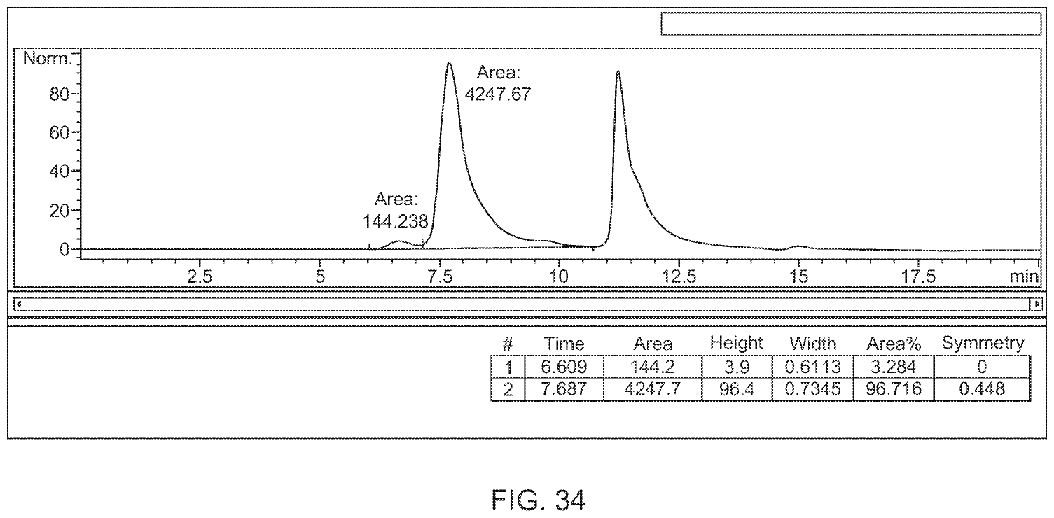

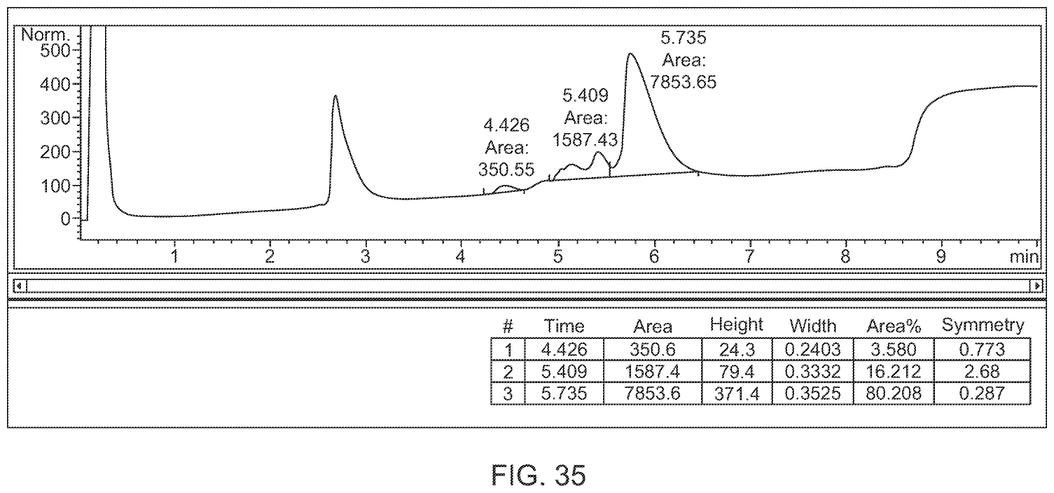

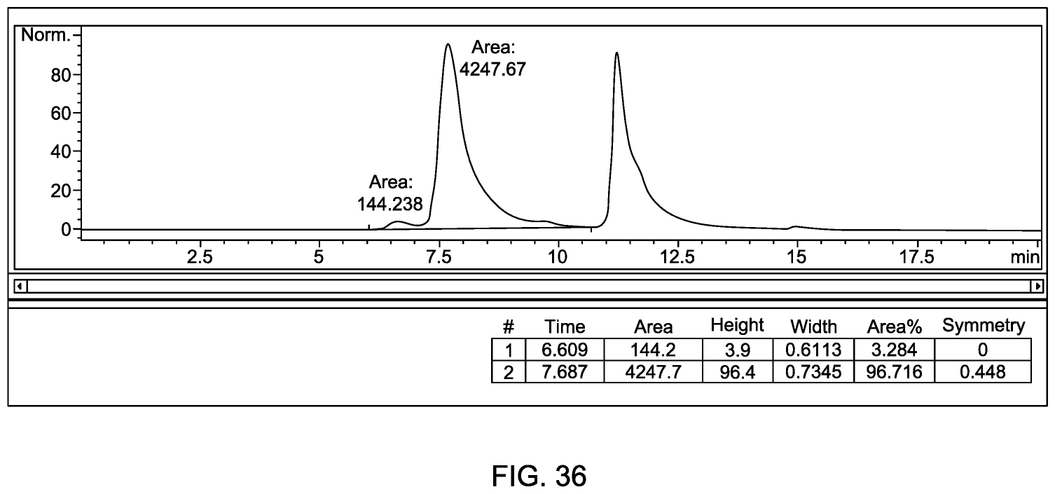

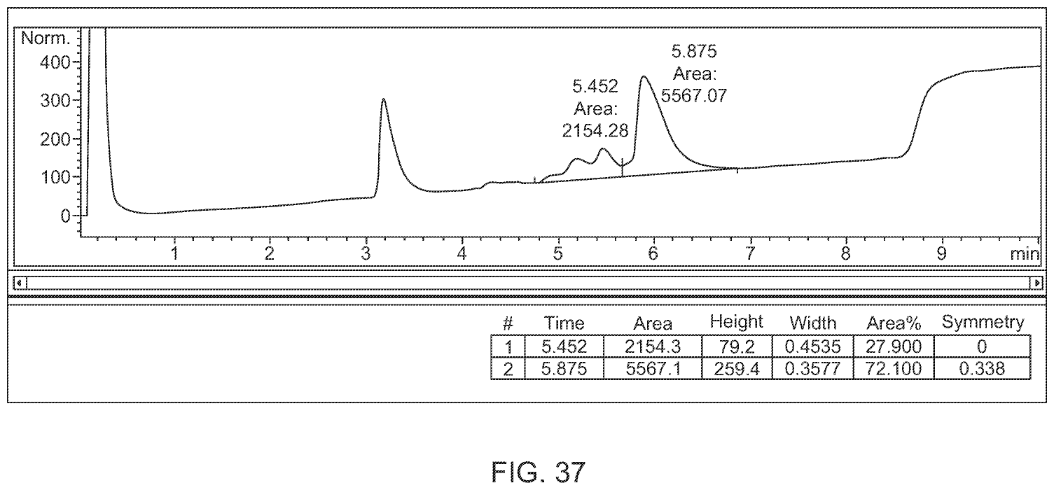

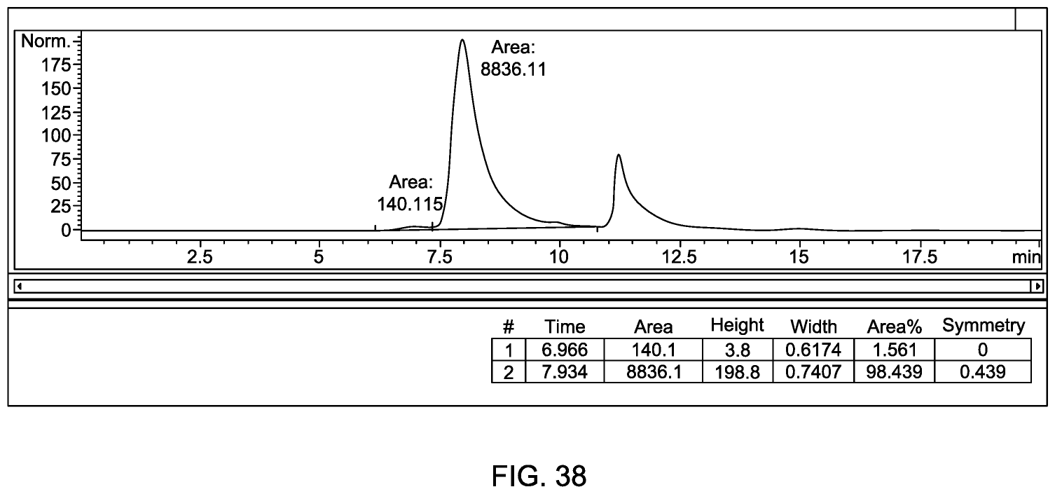

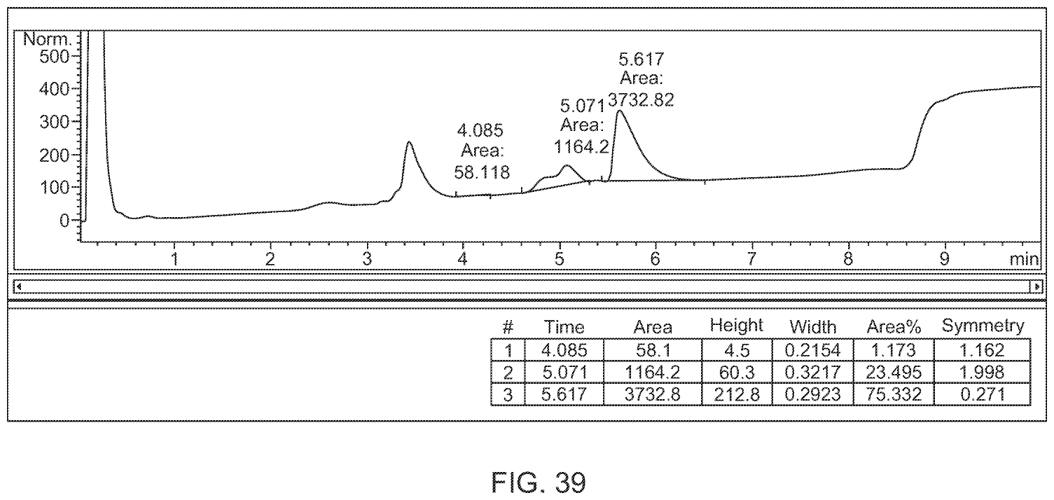

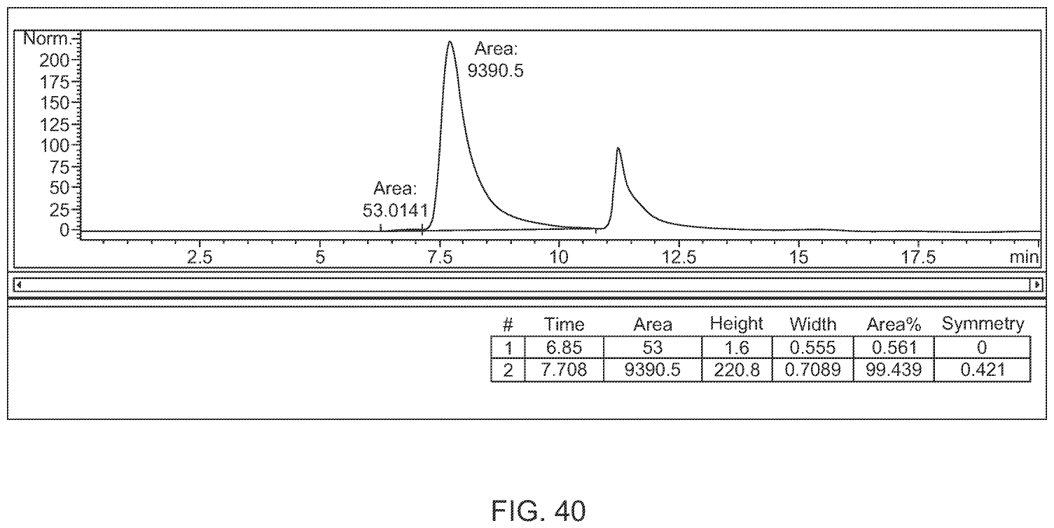

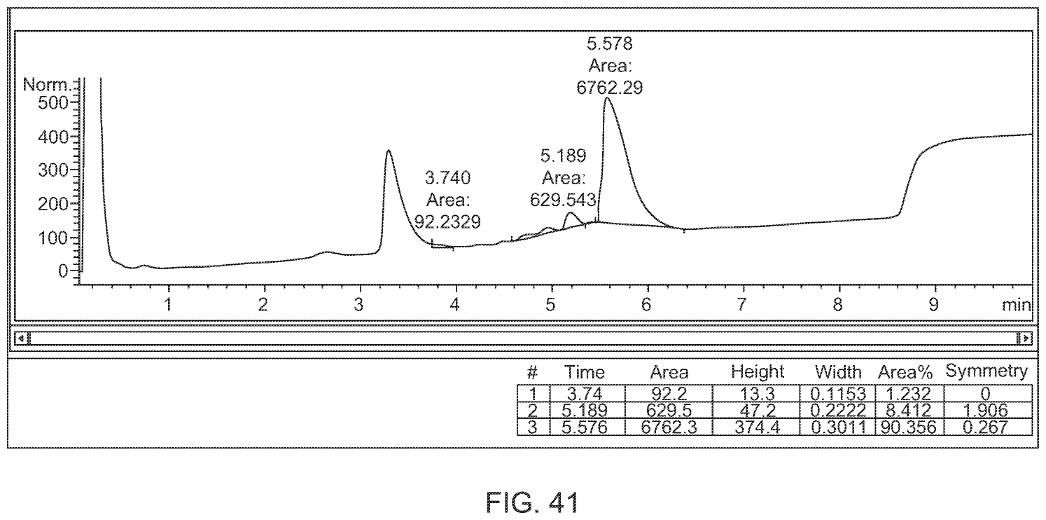

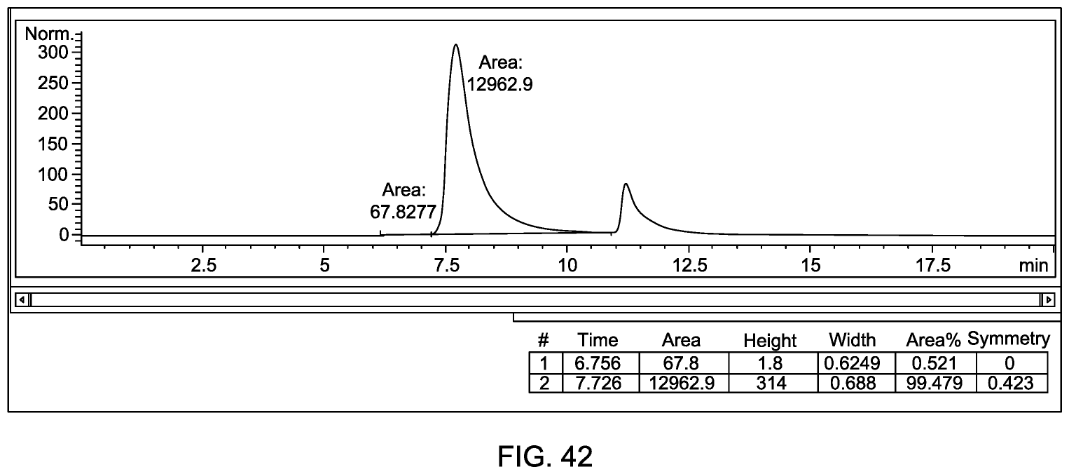

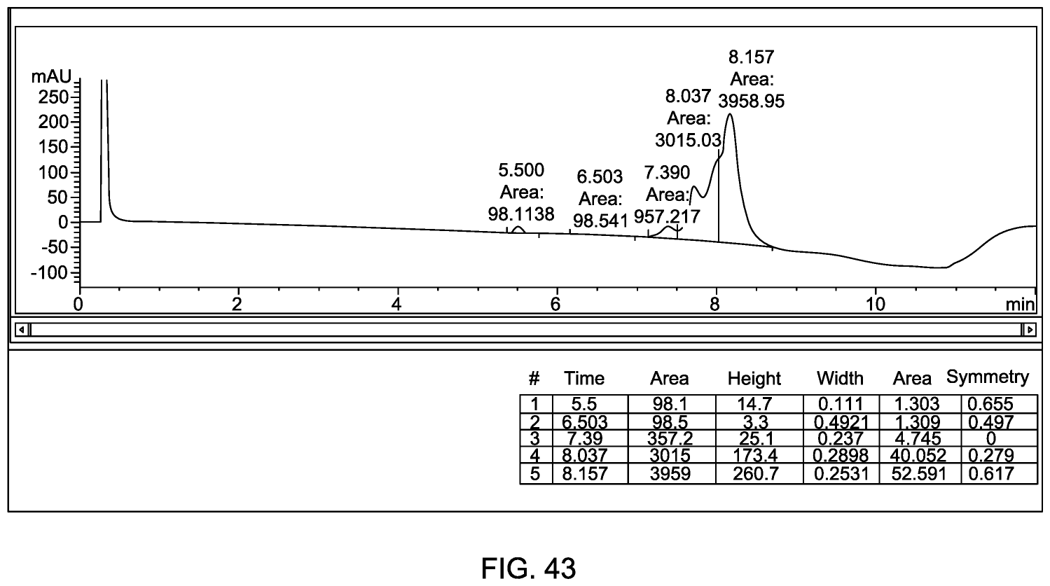

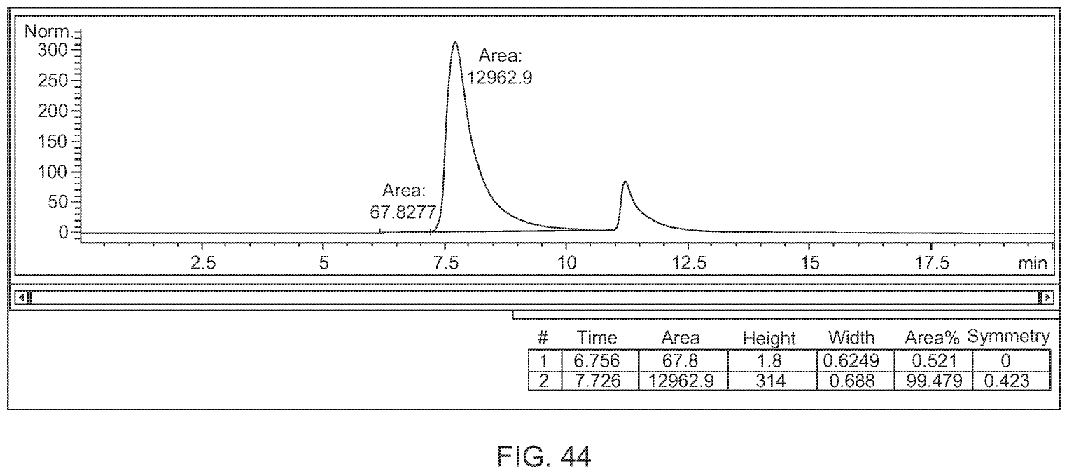

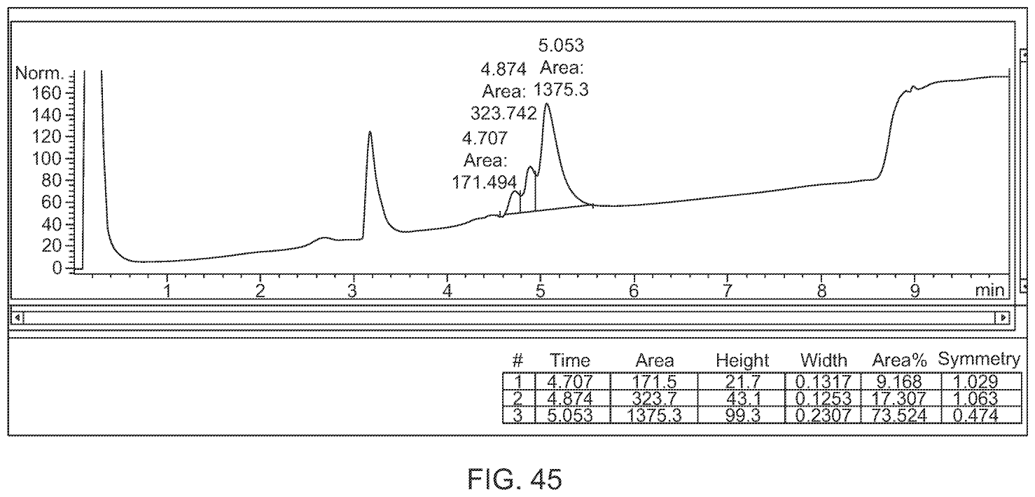

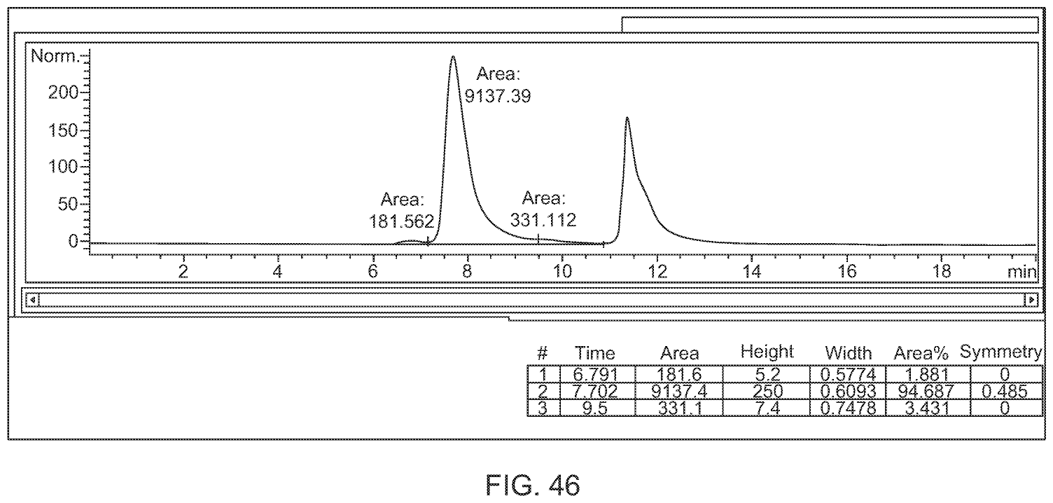

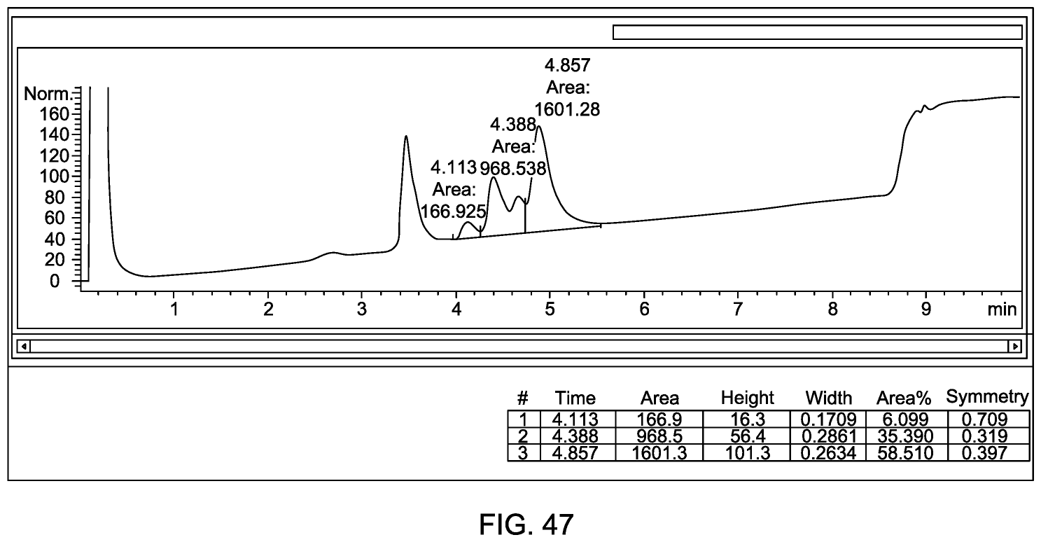

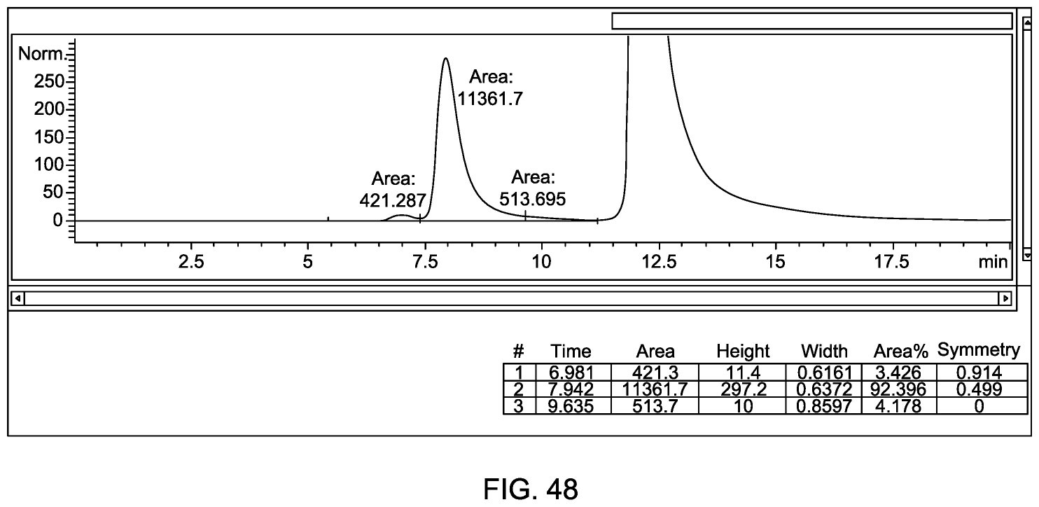

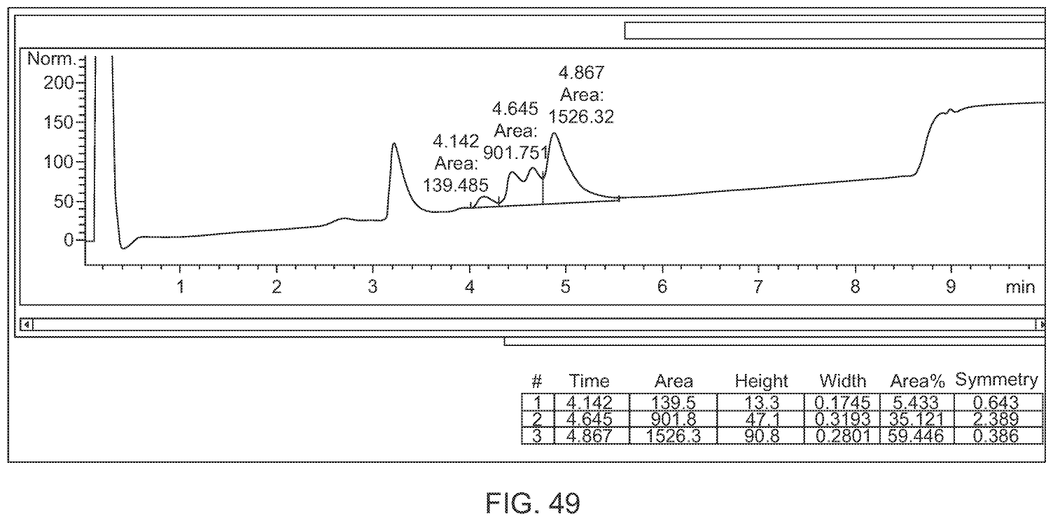

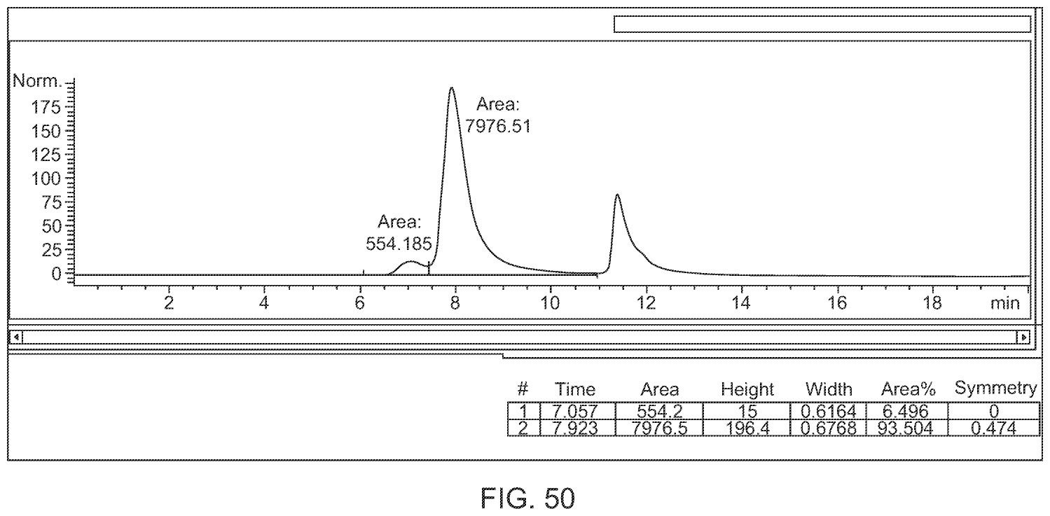

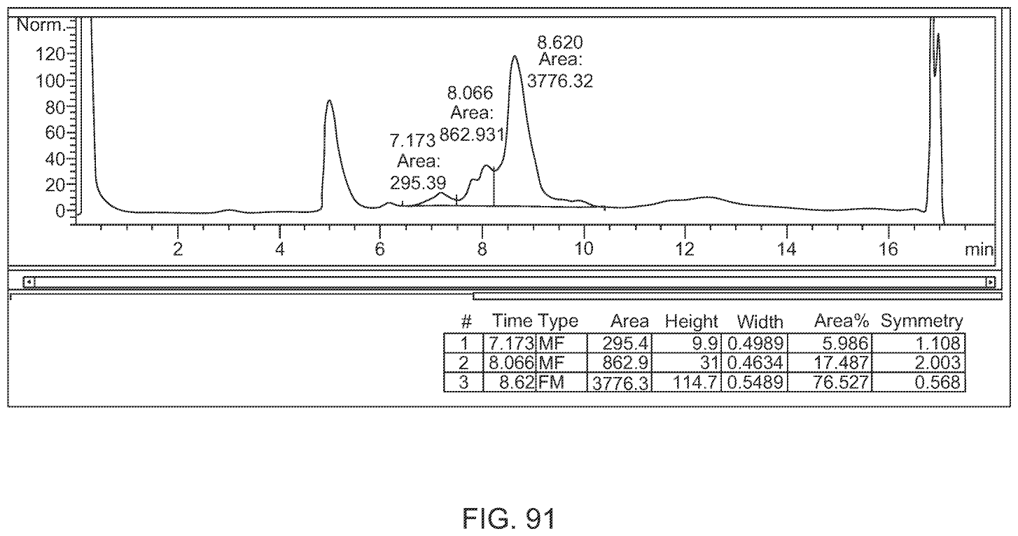

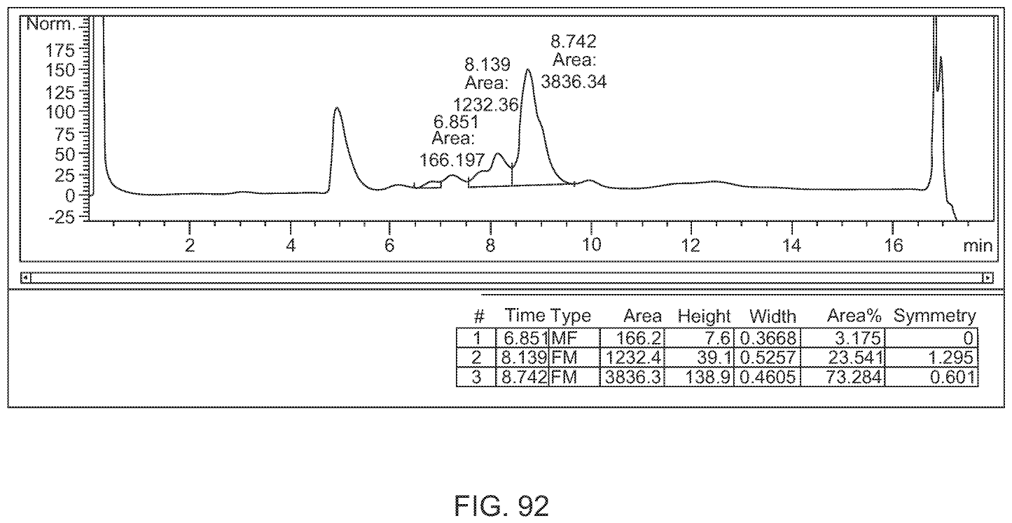

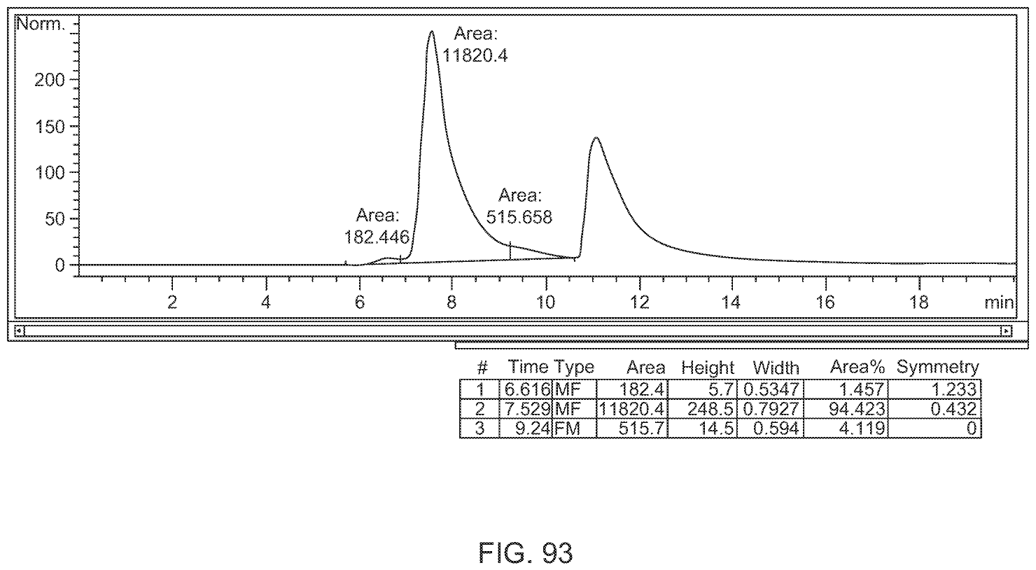

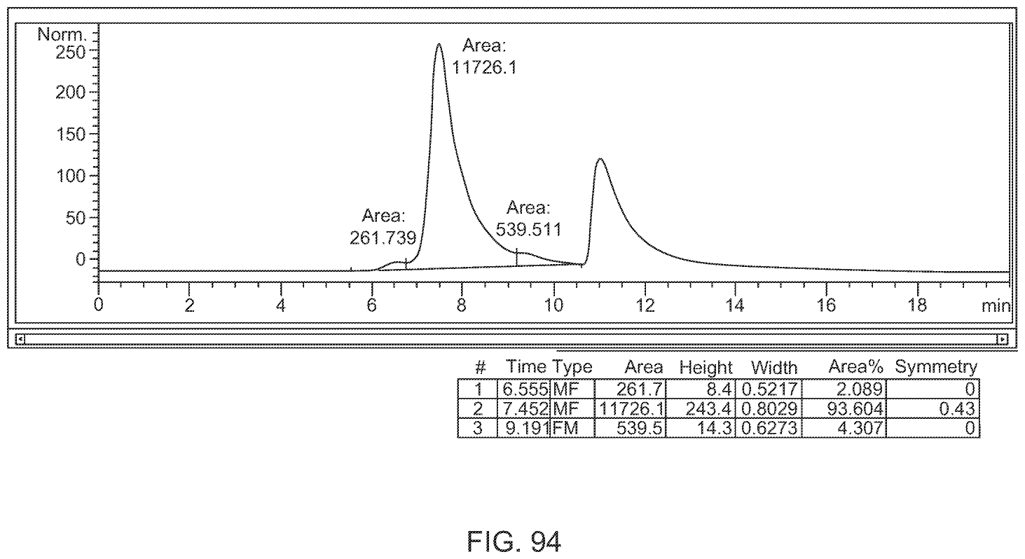

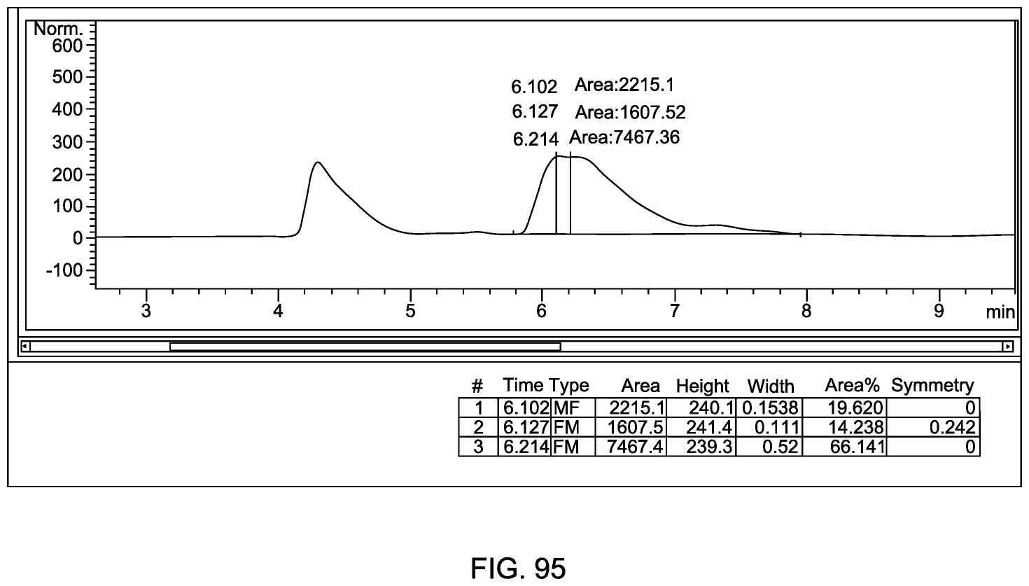

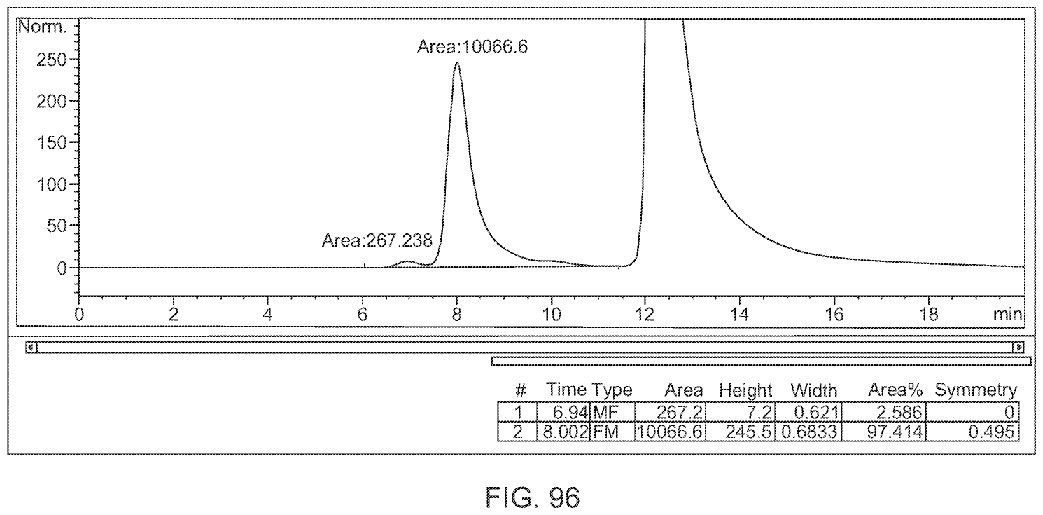

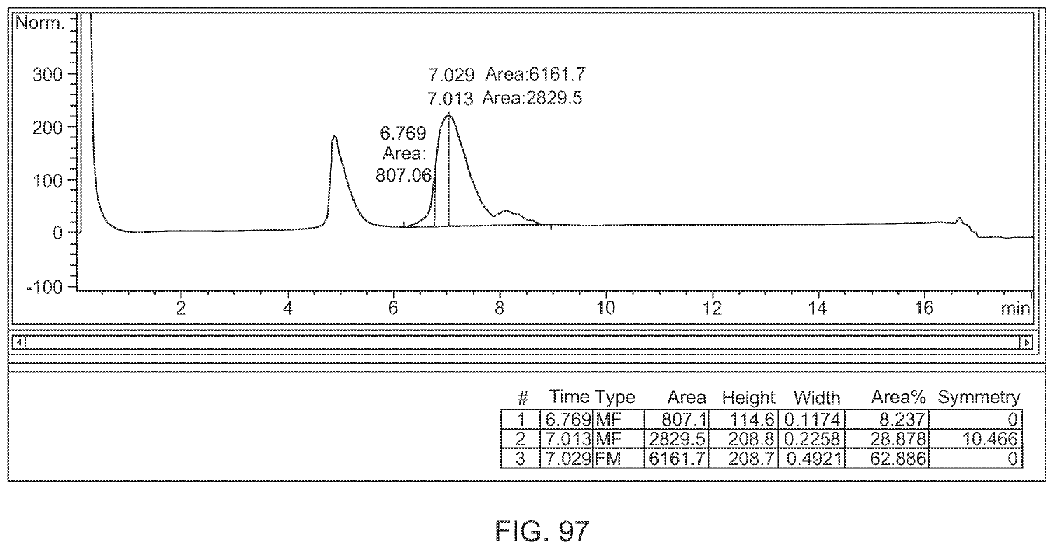

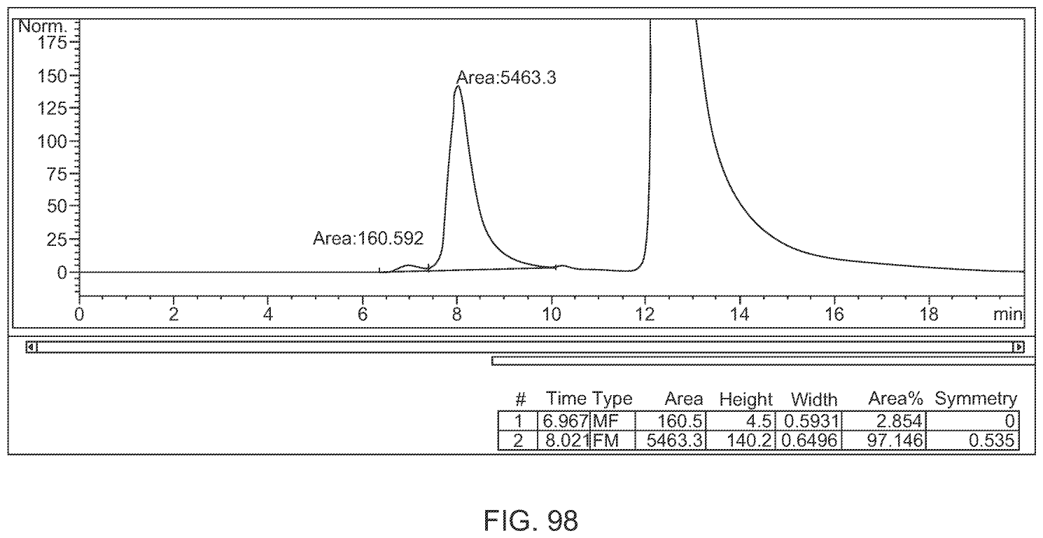

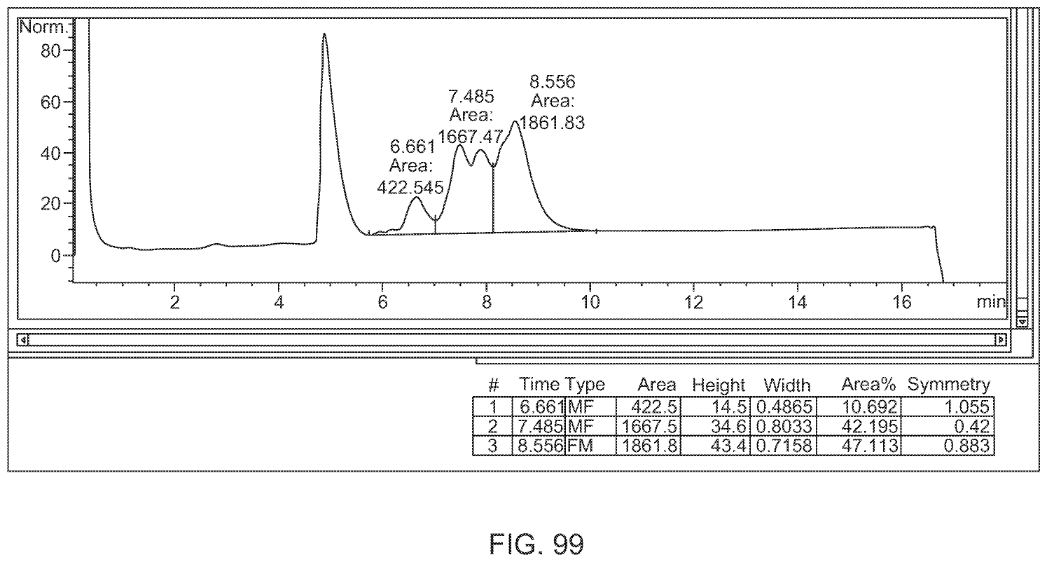

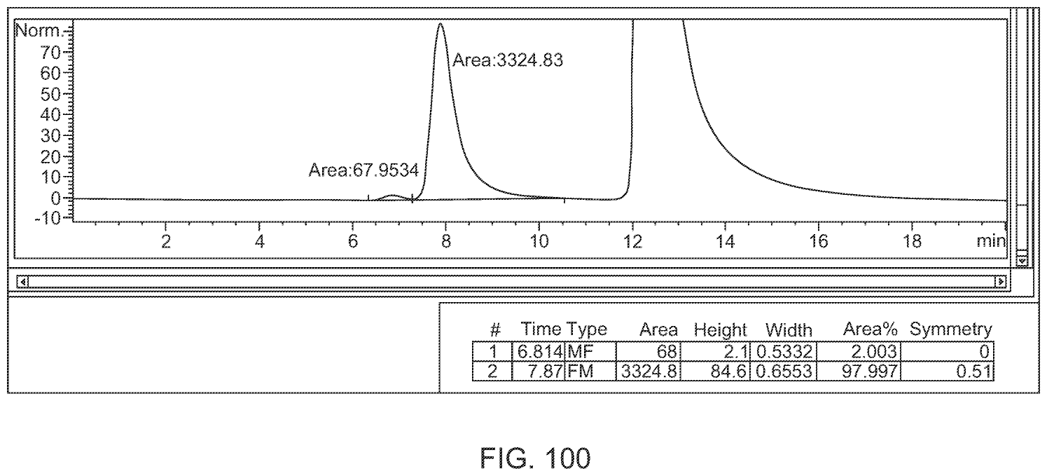

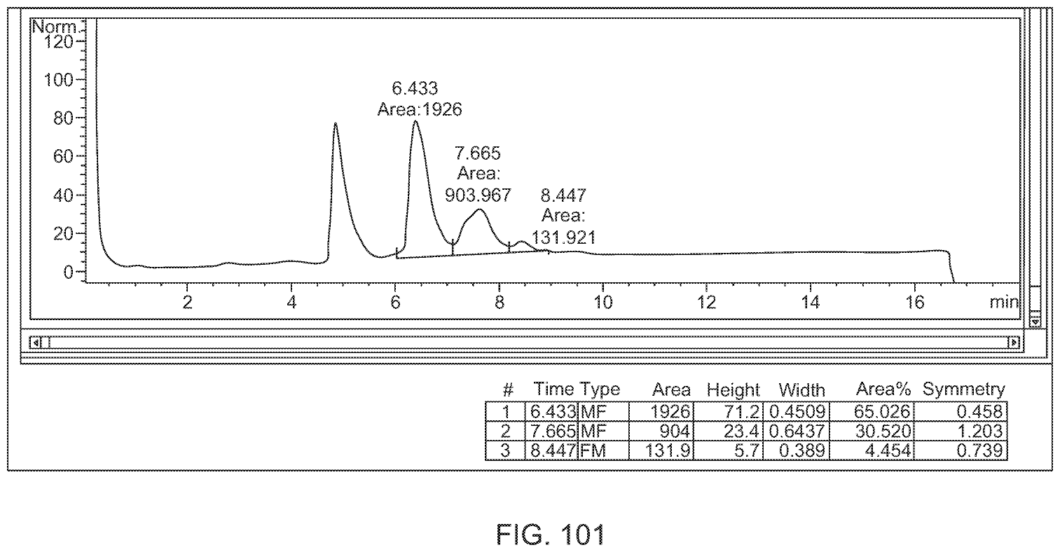

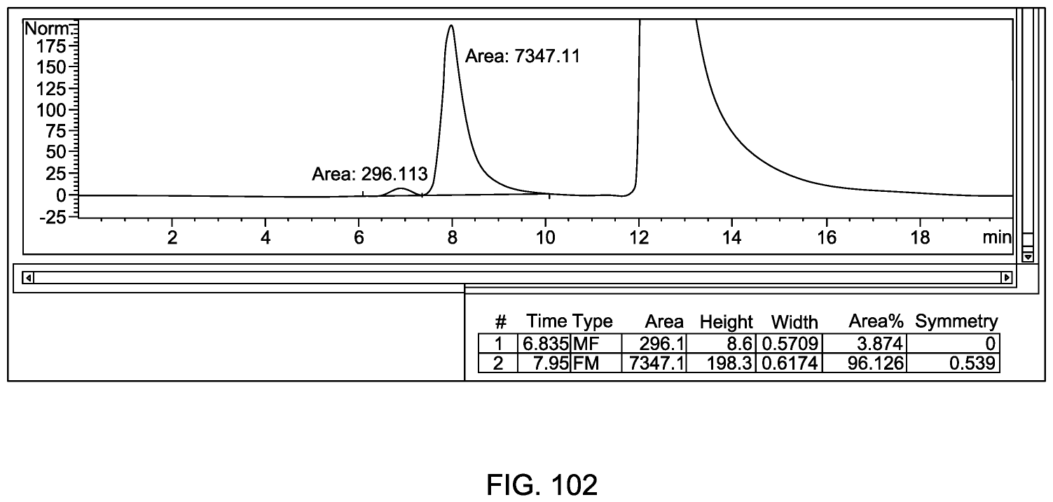

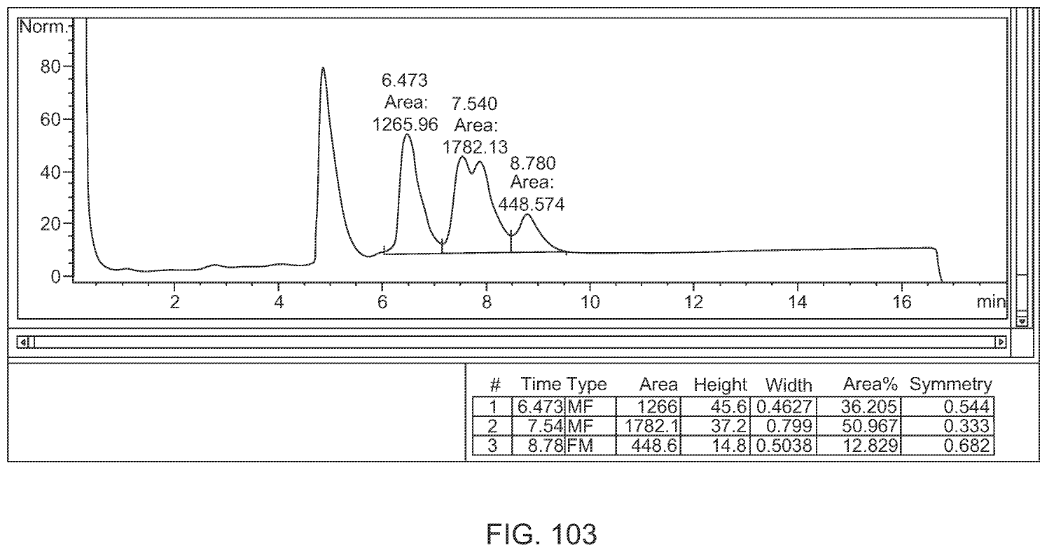

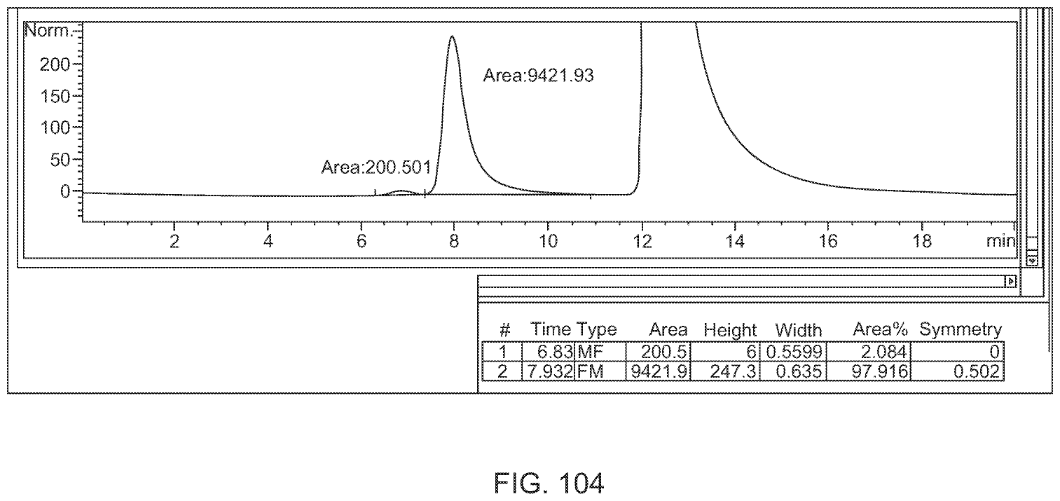

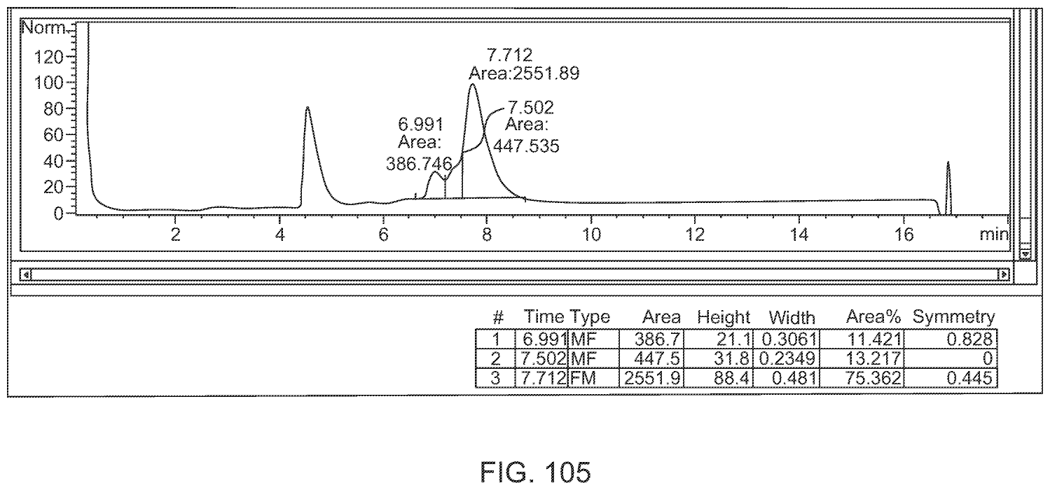

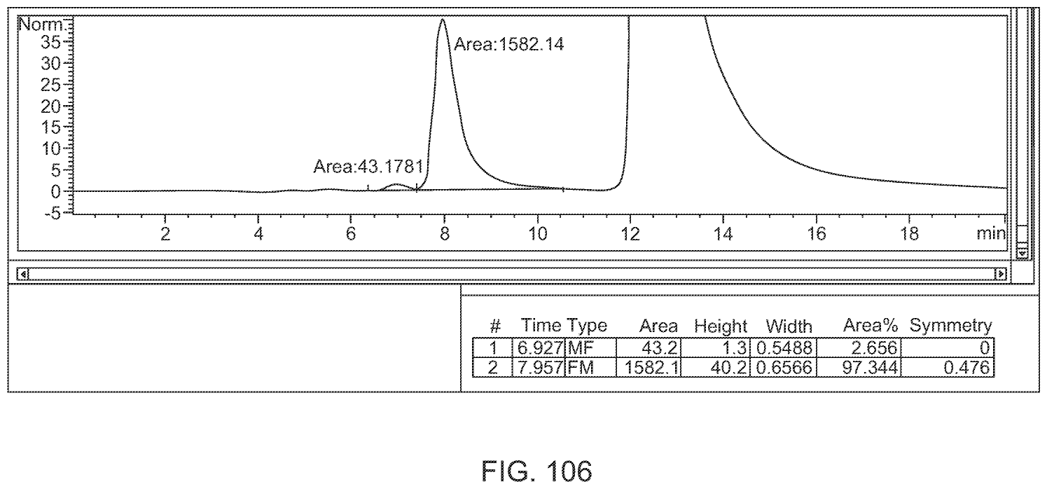

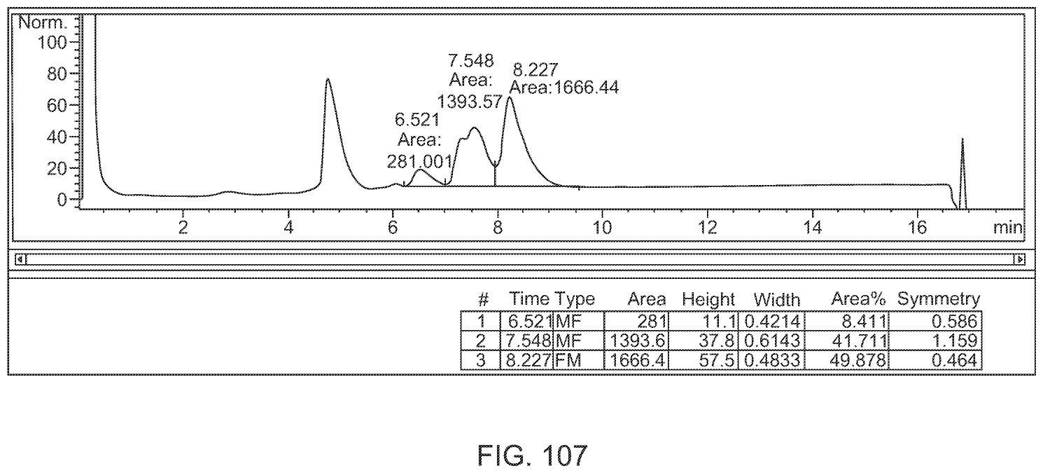

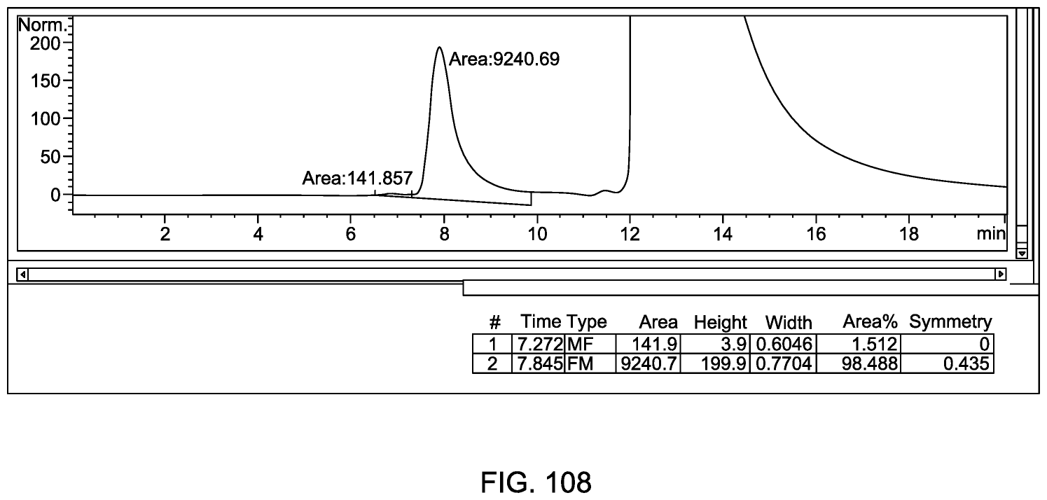

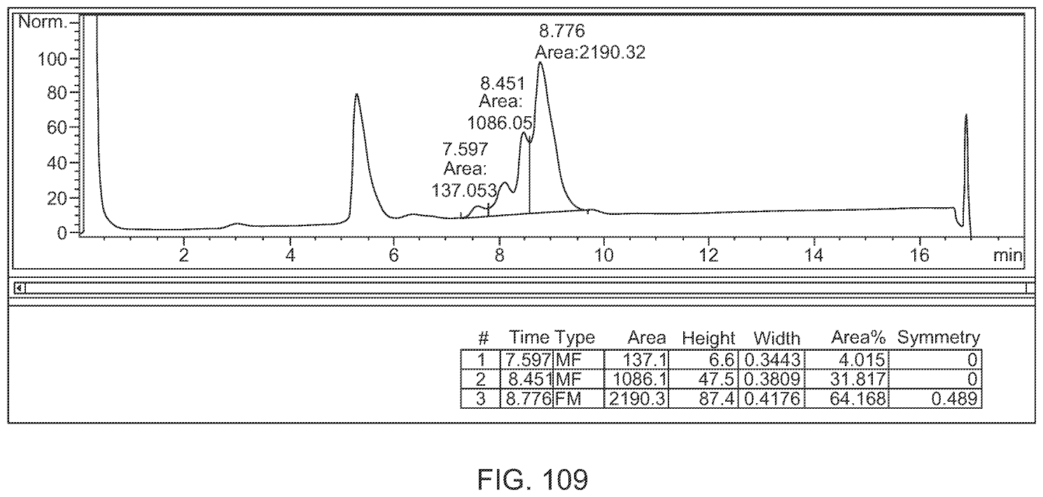

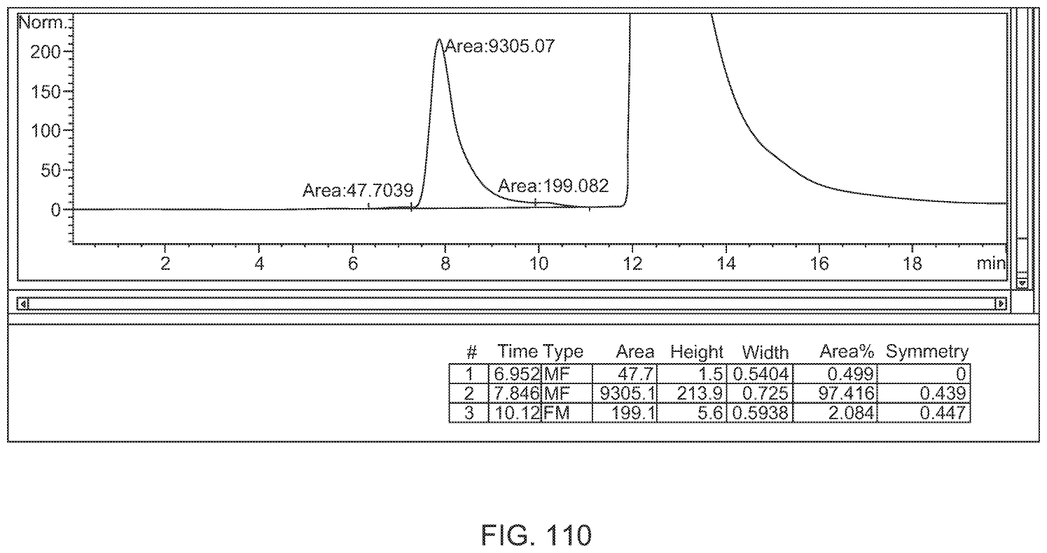

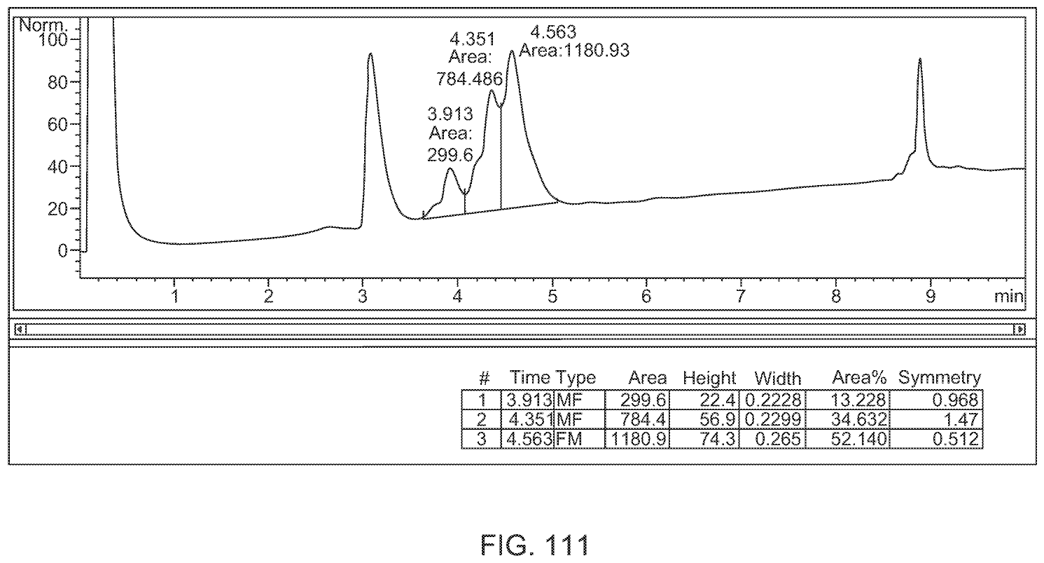

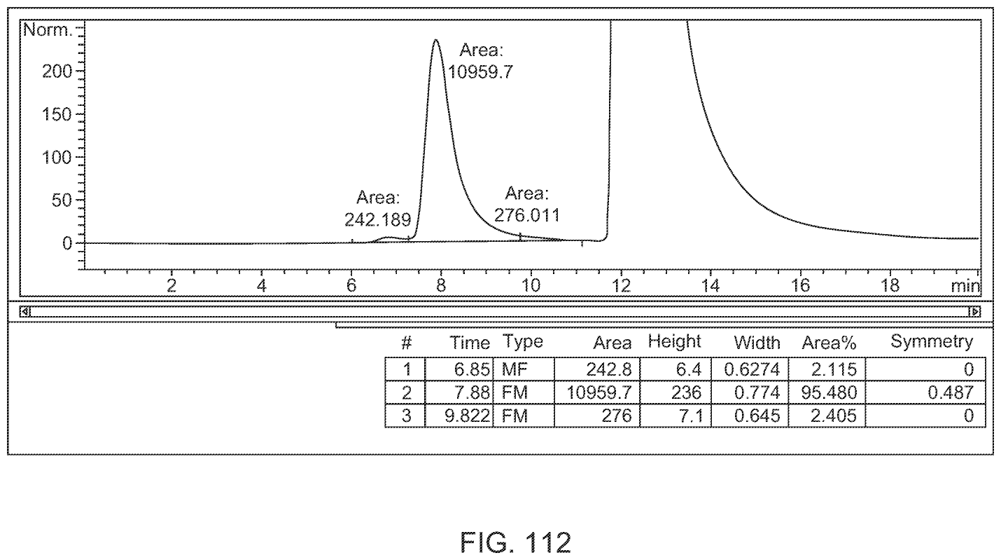

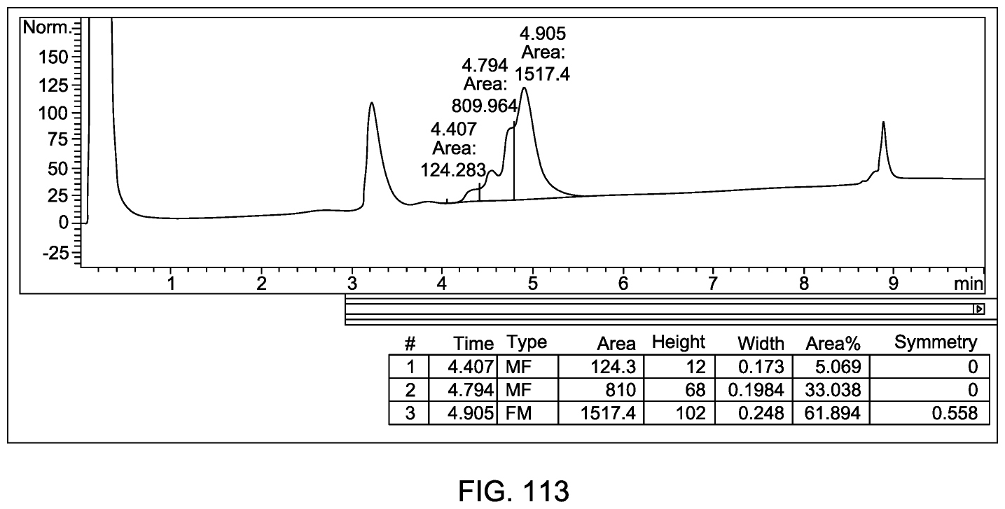

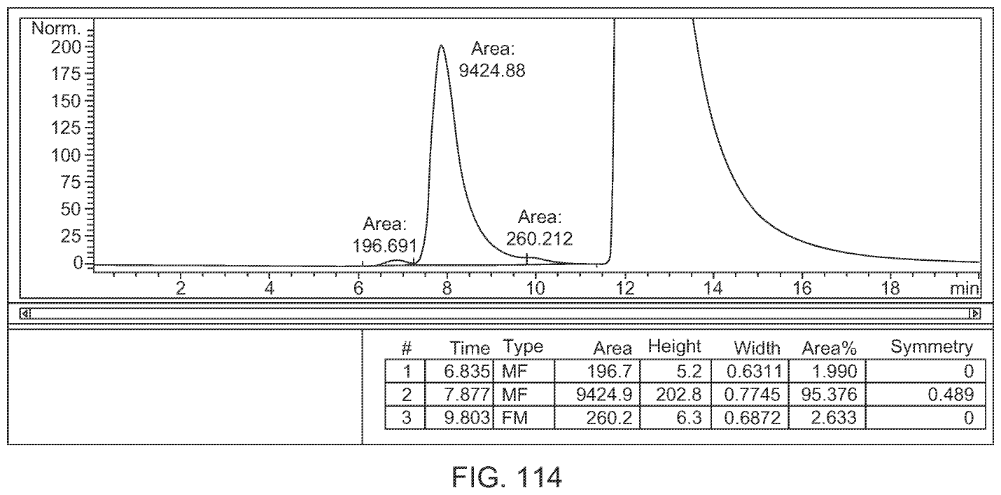

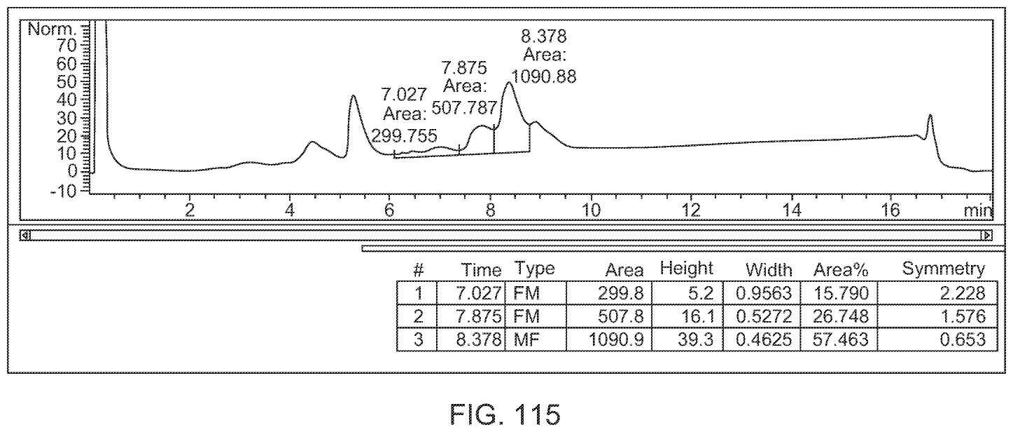

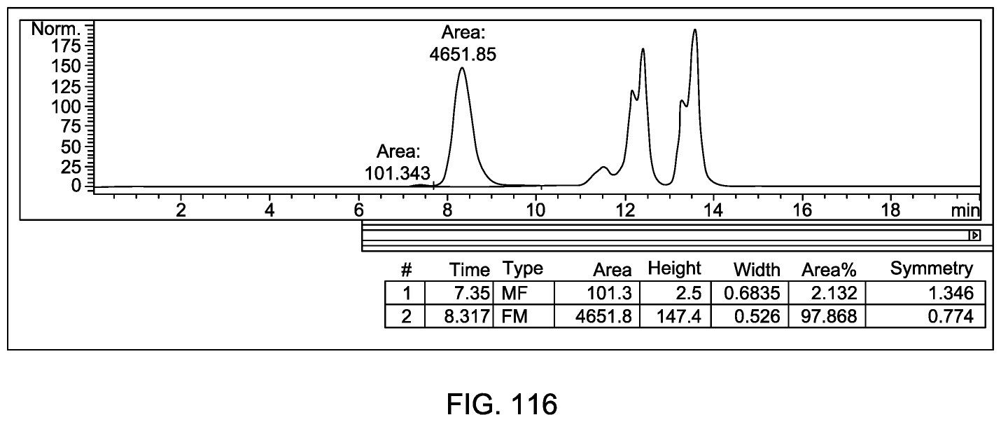

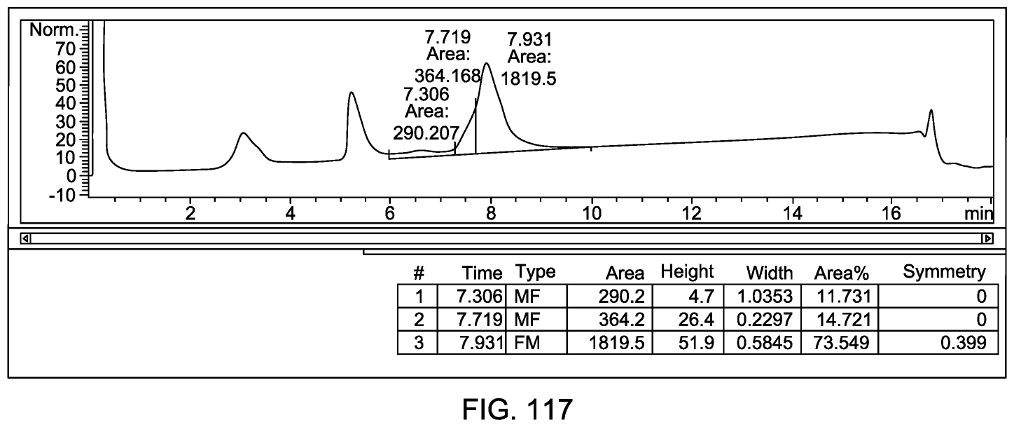

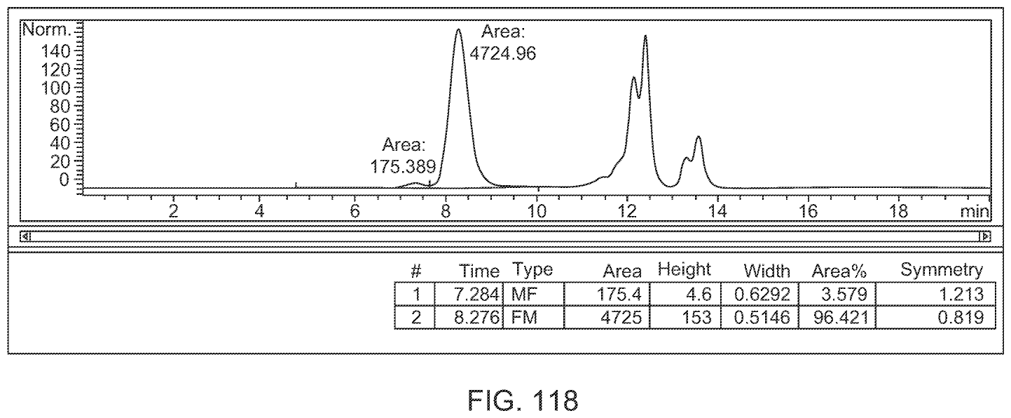

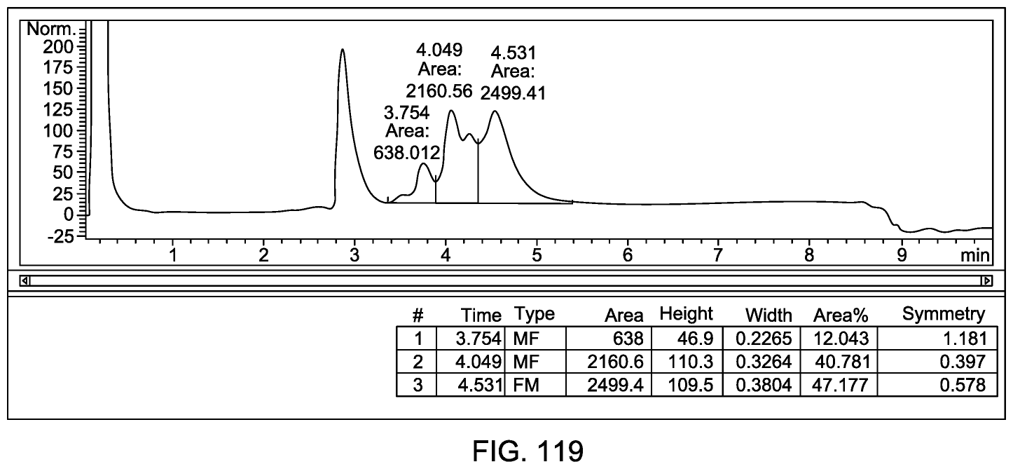

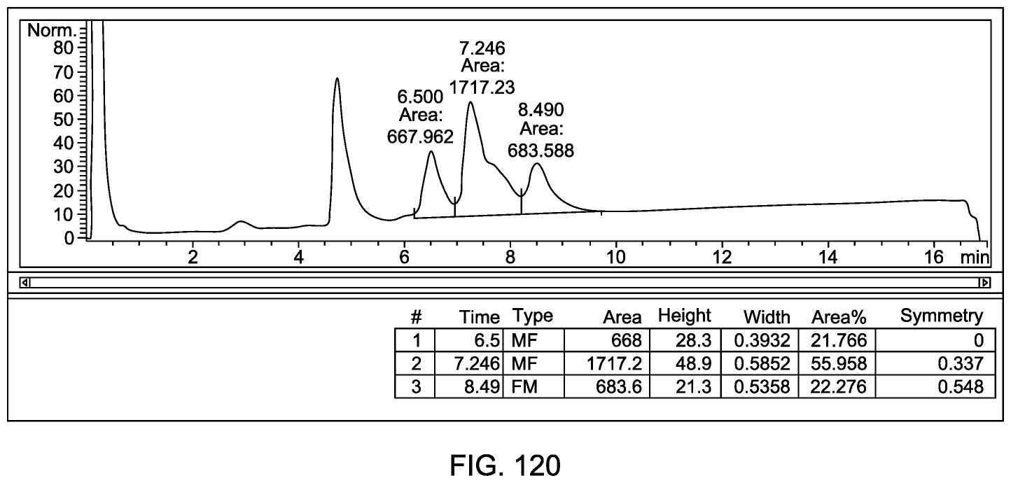

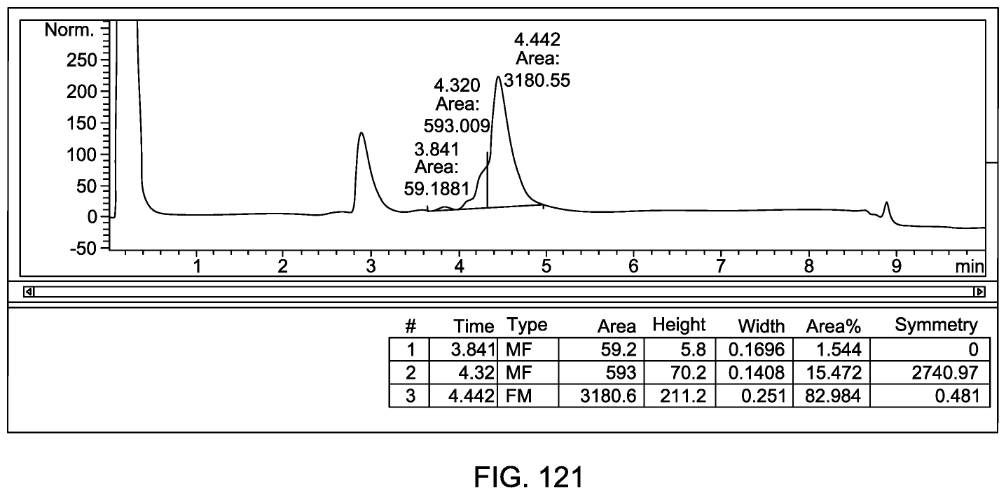

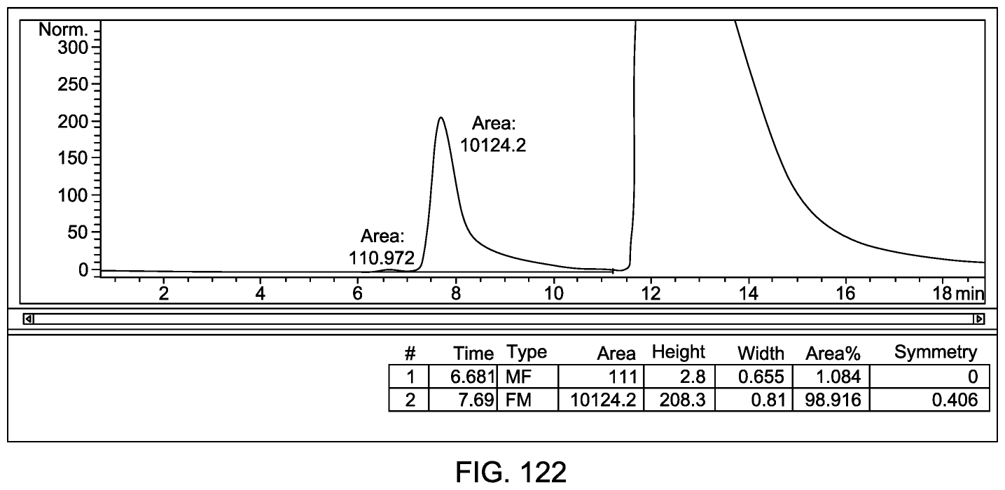

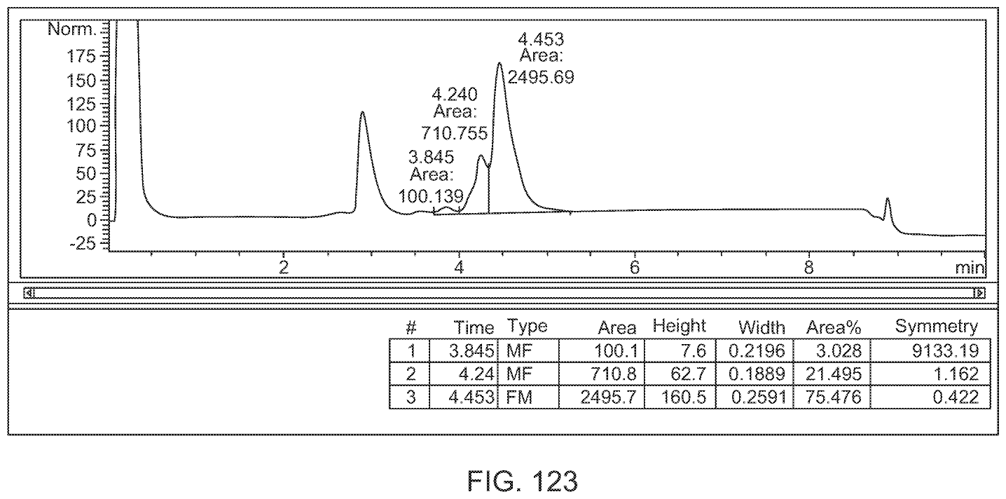

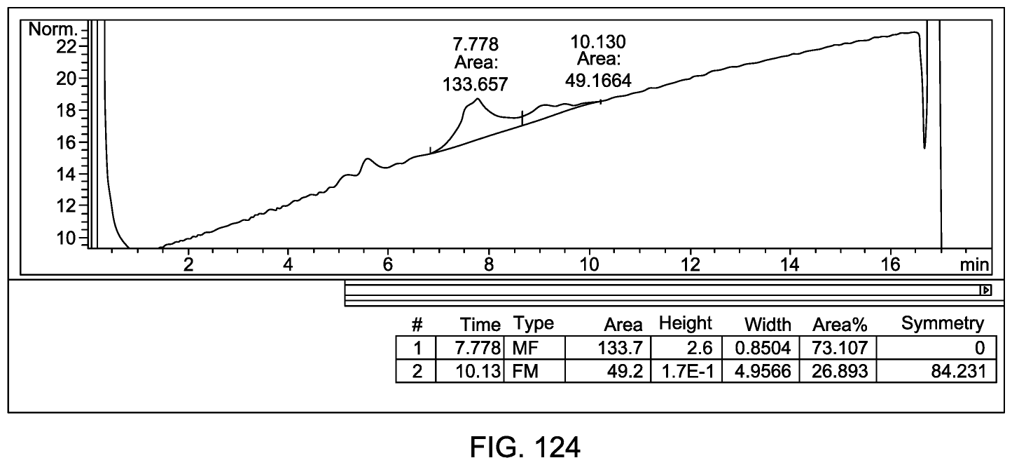

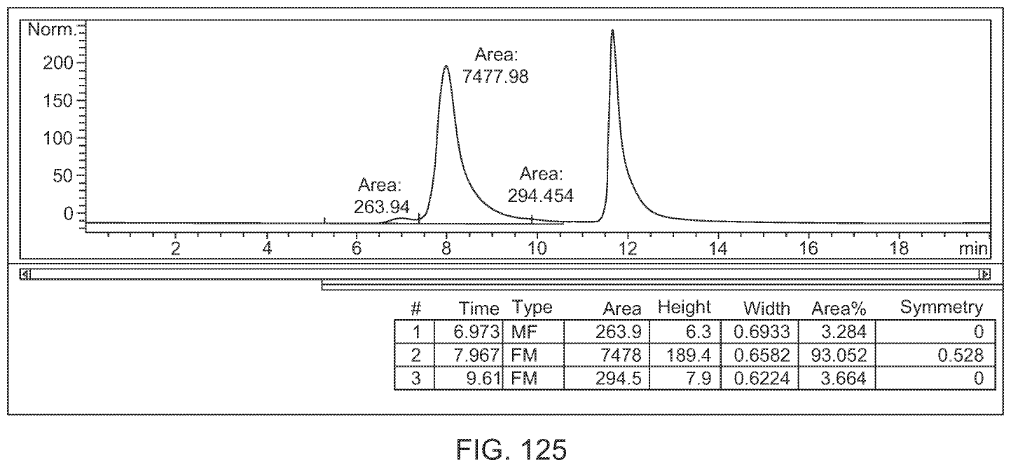

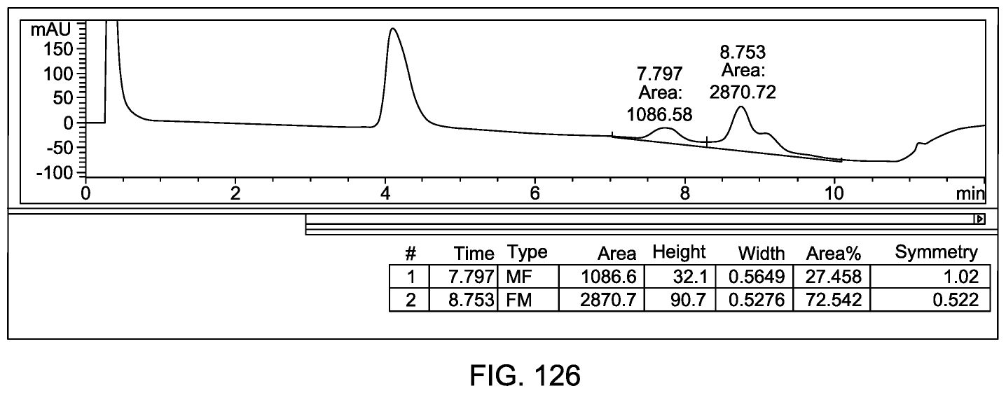

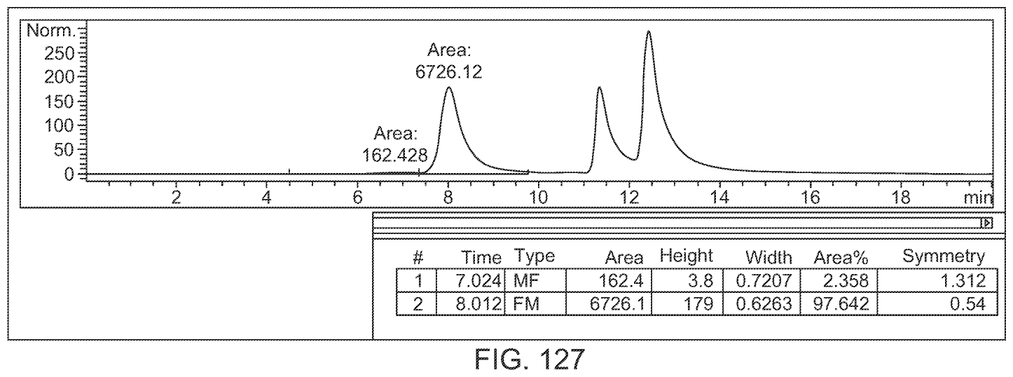

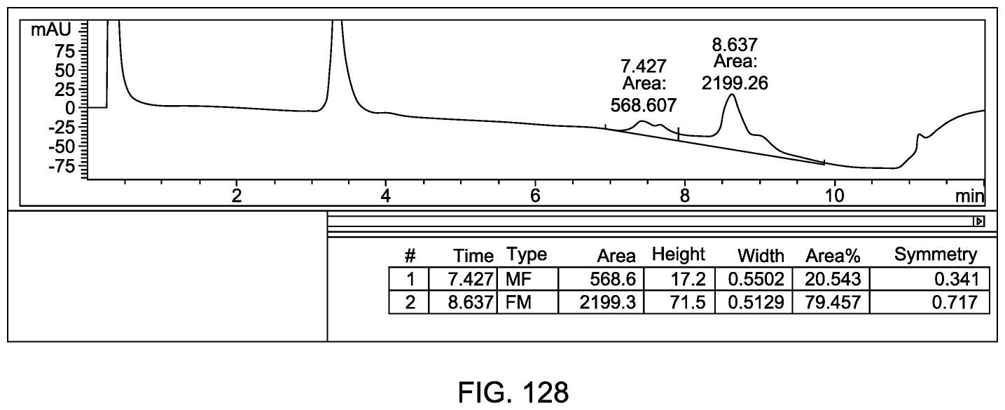

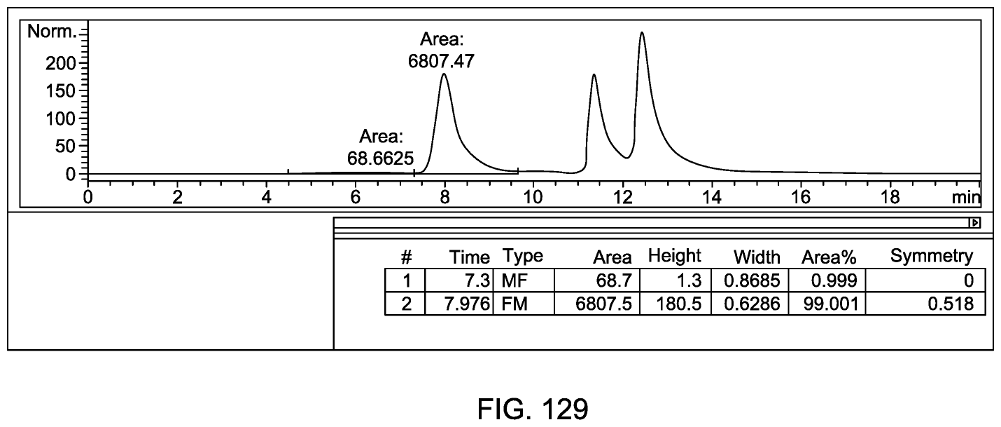

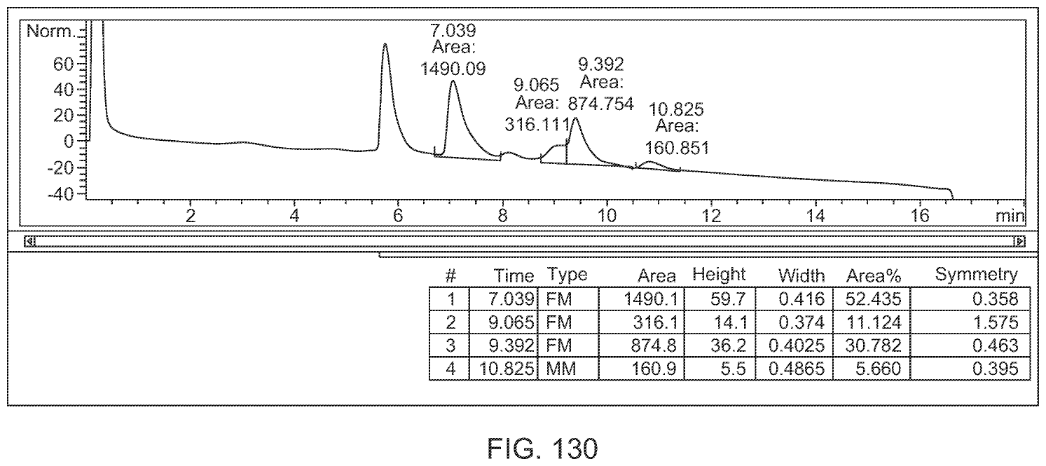

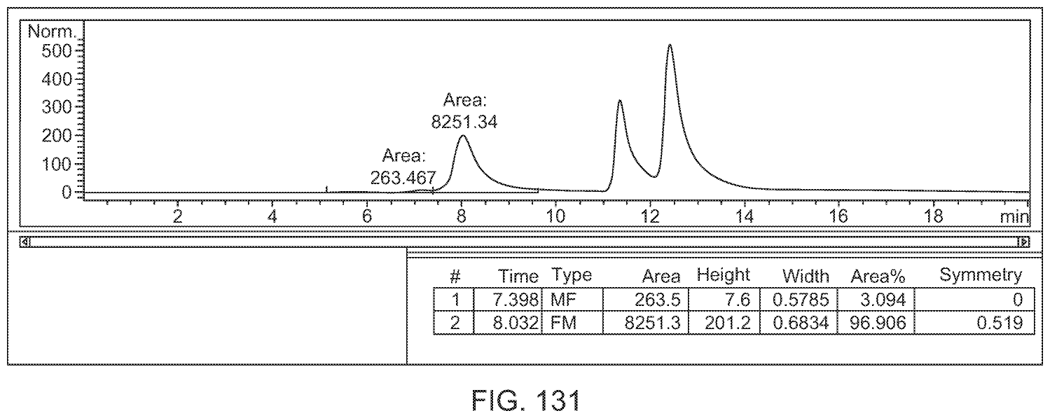

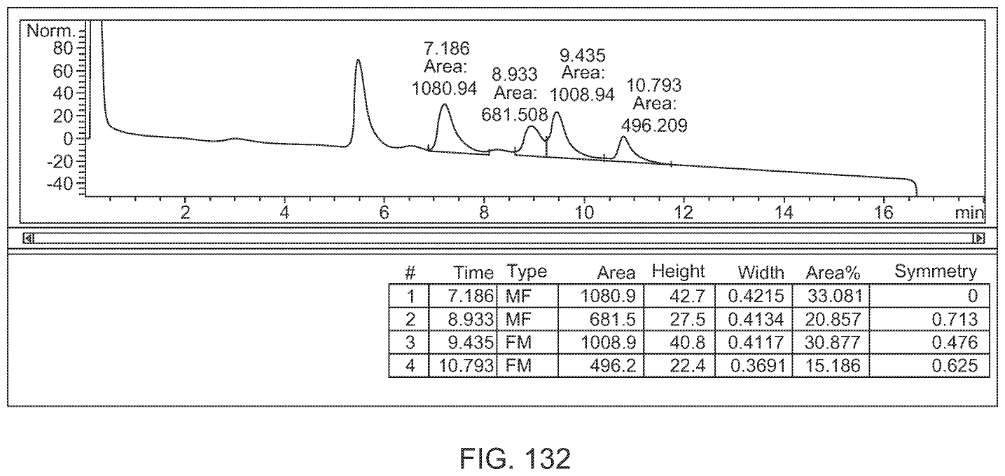

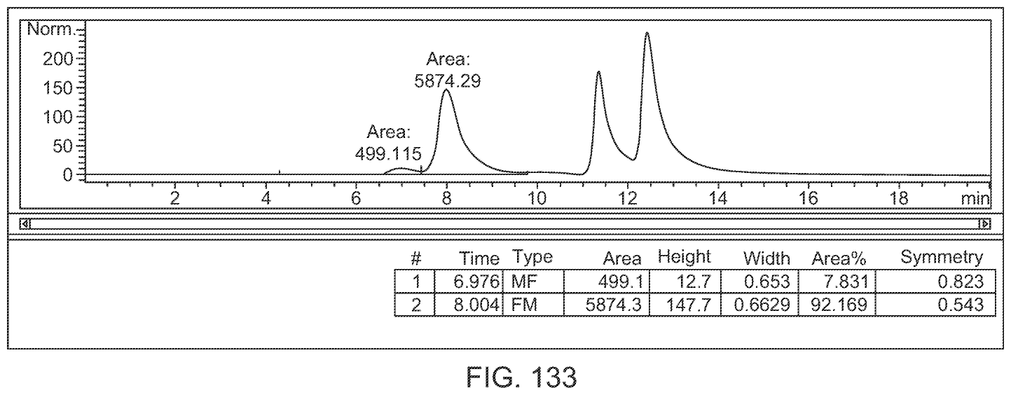

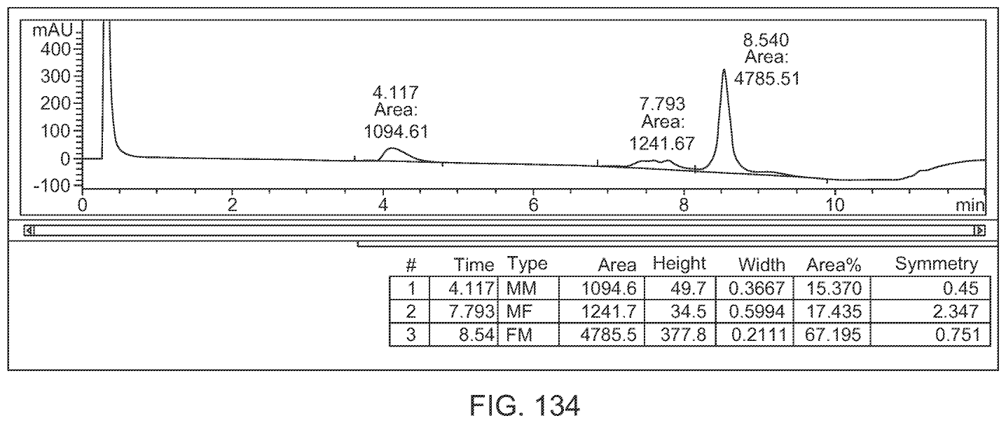

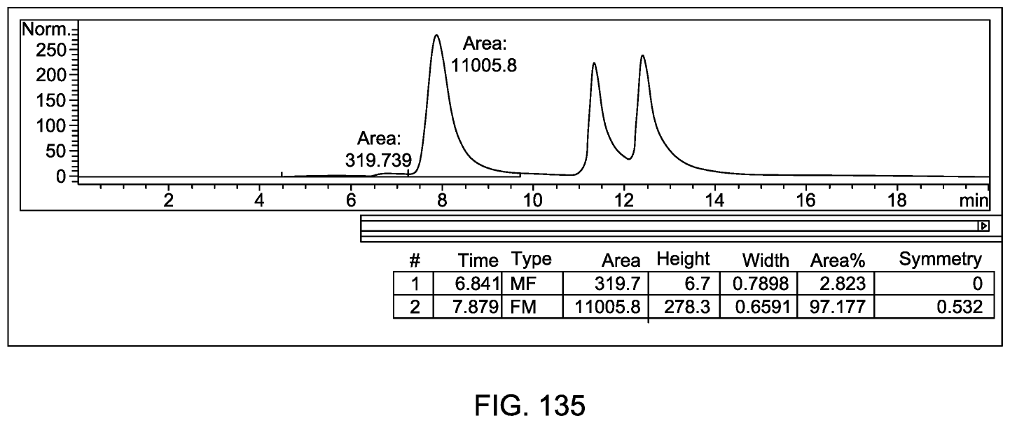

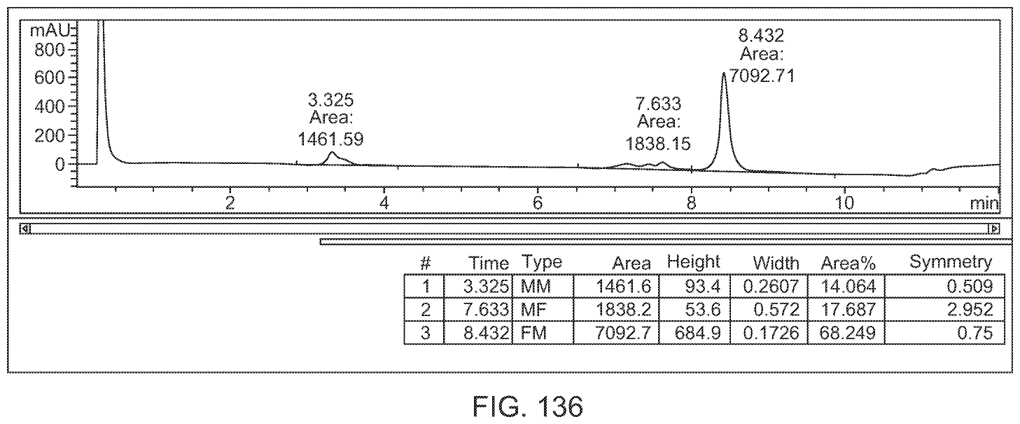

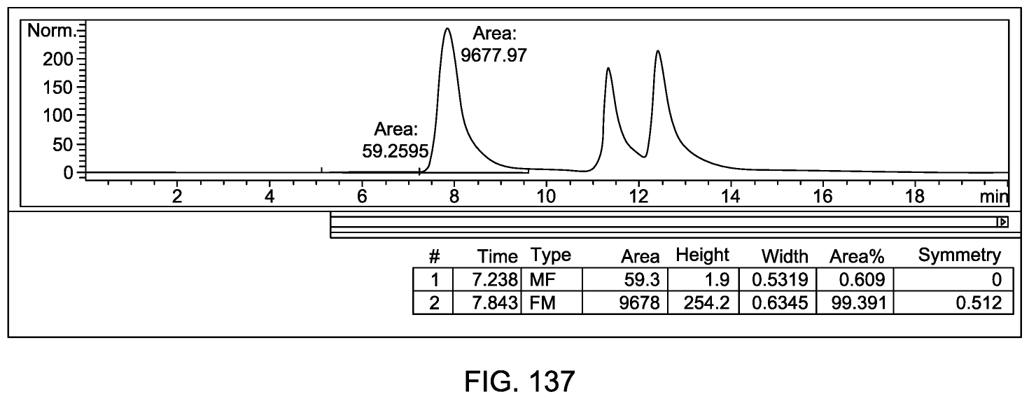

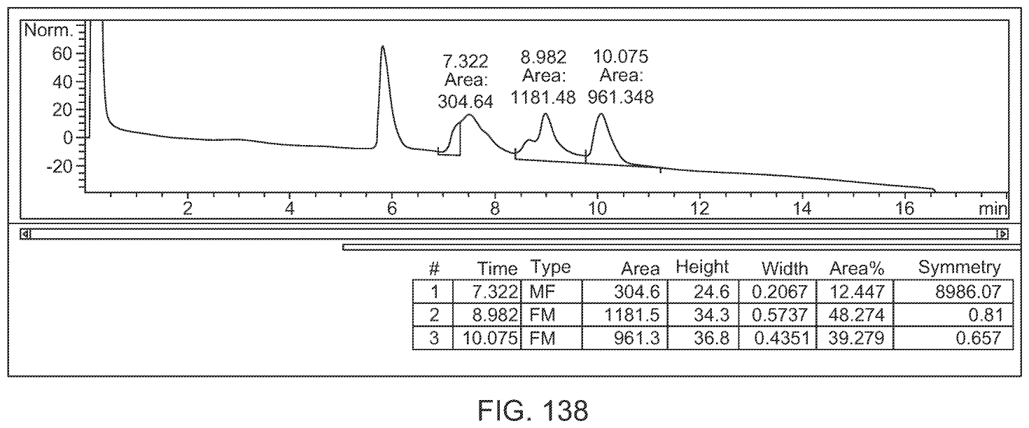

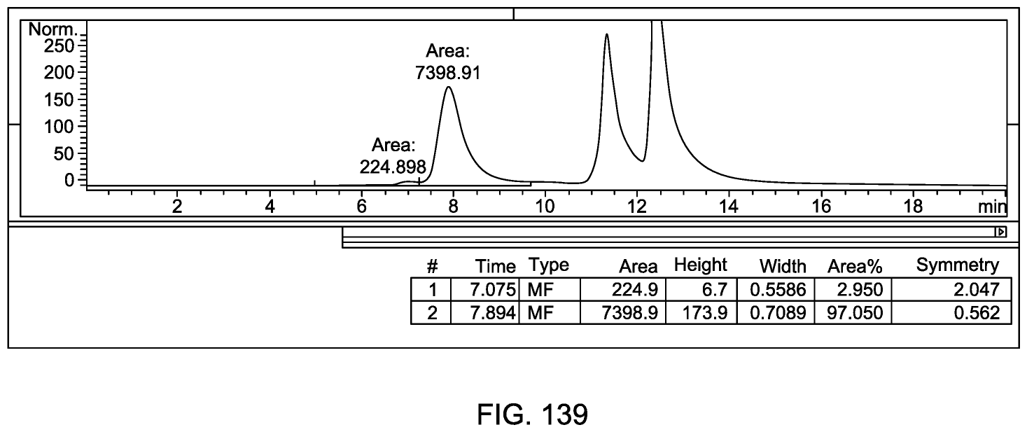

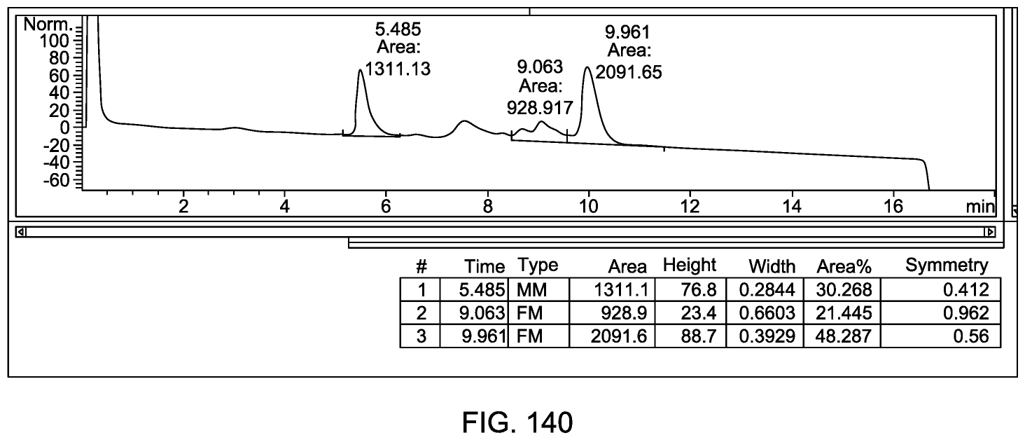

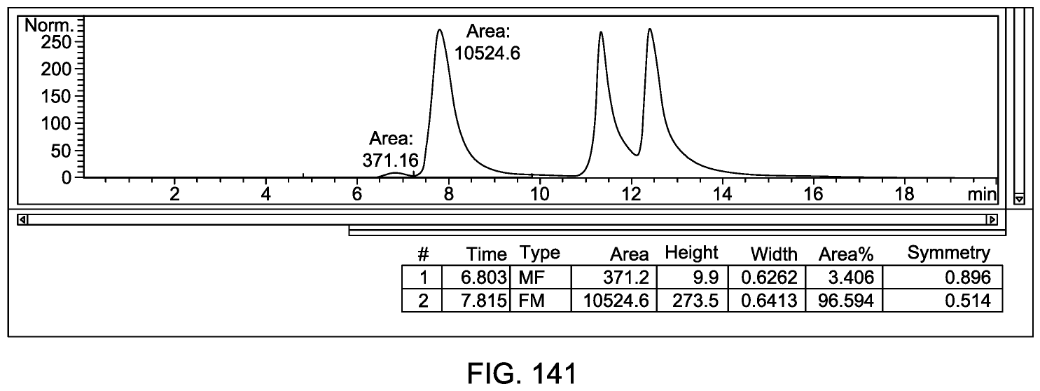

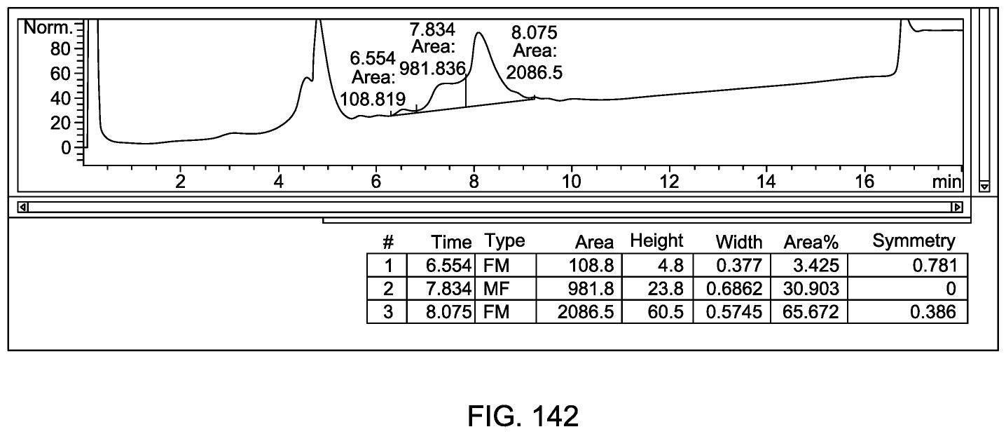

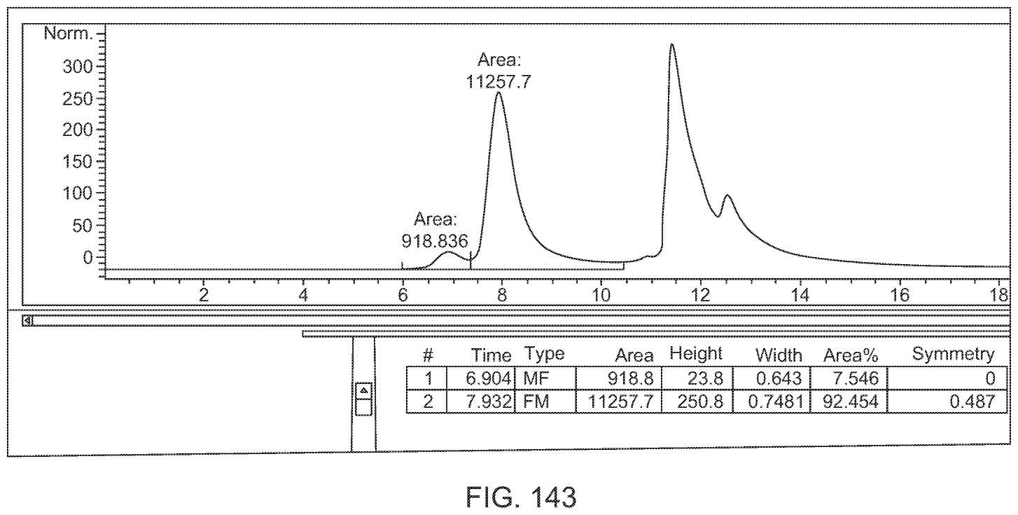

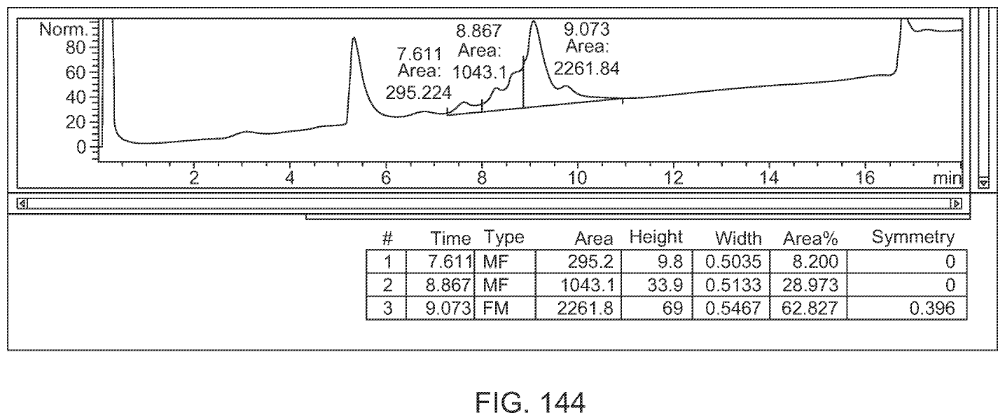

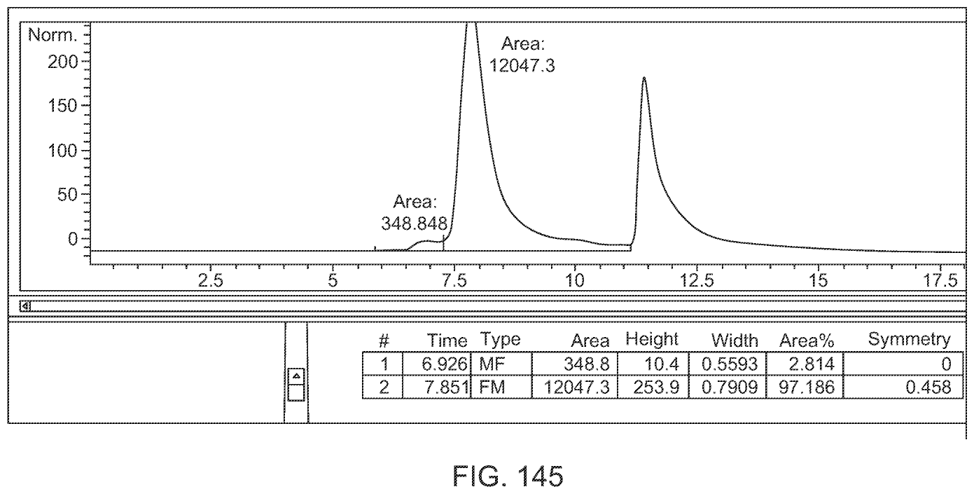

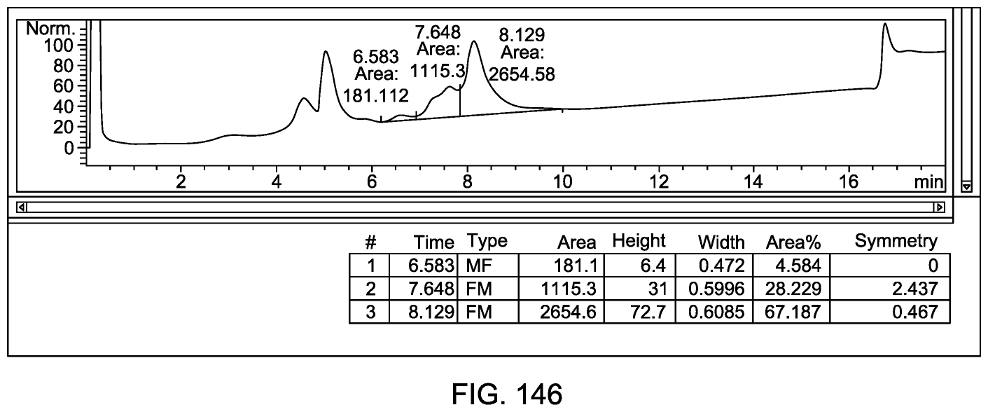

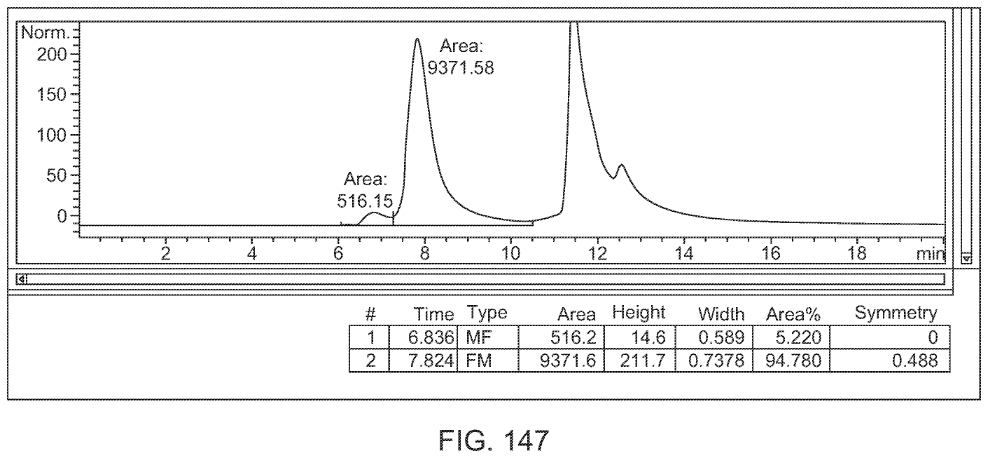

Results of the assays and corresponding DAR values are shown in FIGS. 3-50 and FIGS. 91-147.

Results of conjugation of Compounds 18, 32 and 36 to 10 different antibodies are shown in Table 1 below. Table 1 shows drug-to-antibody (DAR) ratios and % high-molecular weight species (% HMW).

TABLE 1

| Antibody | DAR* | % HMW** | DAR | % HMW | DAR | % HMW |
|---|---|---|---|---|---|---|
| Target | Compound 18 | Compound 18 | Compound 36 | Compound 36 | Compound 32 | Compound 32 |
| Target 1 | 6.82 | 1.8 | n.d. | n.d. | n.d. | n.d. |
| Target 2 | 6.16 | 6.5 | 6.78 | 4.1 | 6.84 | 2.3 |
| Target 3 | 4.31 | 4.3 | 6.33 | 6.1 | 6.88 | 2.2 |
| Target 4 | 6.97 | 0.6 | n.d. | n.d. | n.d. | n.d. |
| Target 5 | 5.45 | 0.8 | 6.88 | 3.3 | 6.93 | 3.2 |
| Target 6 | 7.07 | 2.0 | n.d. | n.d. | n.d. | n.d. |
| Target 7 | 6.88 | 1.6 | n.d. | n.d. | n.d. | n.d. |
| Target 8 | 6.1 | 3.4 | 6.55 | 3.5 | 7.38 | 1.4 |
| Target 9 | 6.84 | 1.5 | 6.32 | 2.2 | 6.99 | 3.0 |
| Target 10 | 5.49 | 2.7 | 6.33 | 6.2 | 7.06 | 2.0 |

*DAR, drug-to-antibody ratio
**HMW, high-molecular weight species

### Example 6

In vitro Cytotoxicity Assays

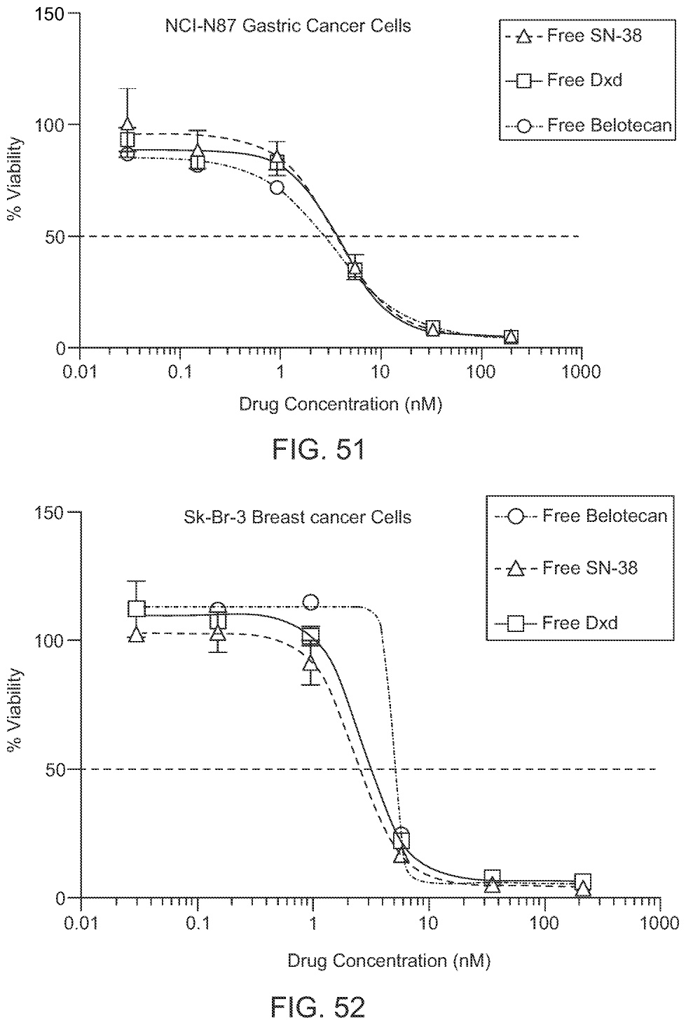

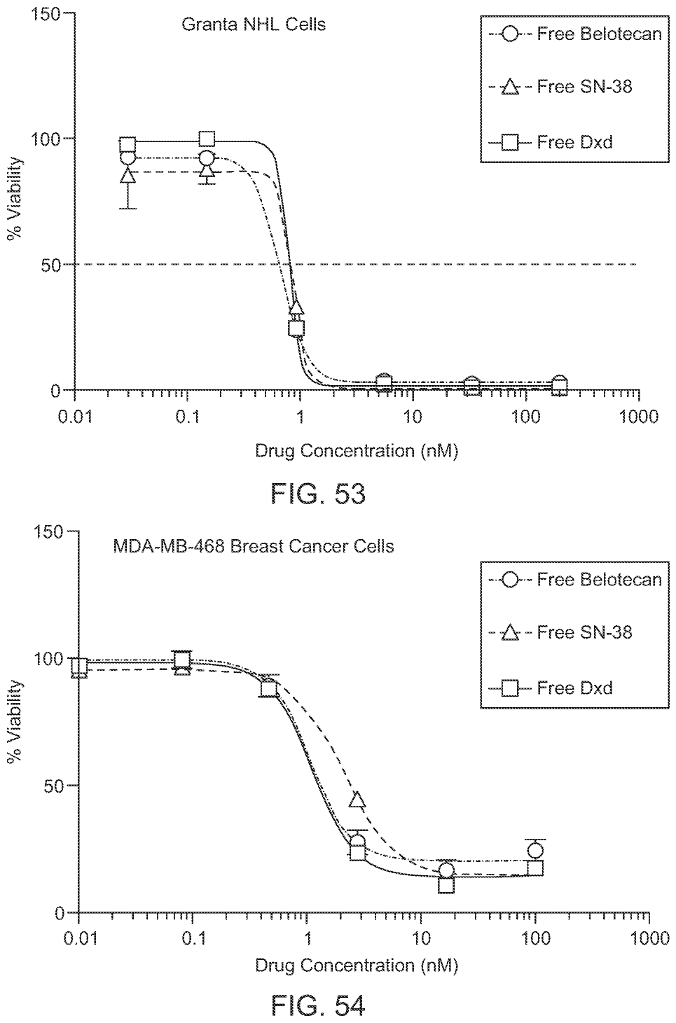

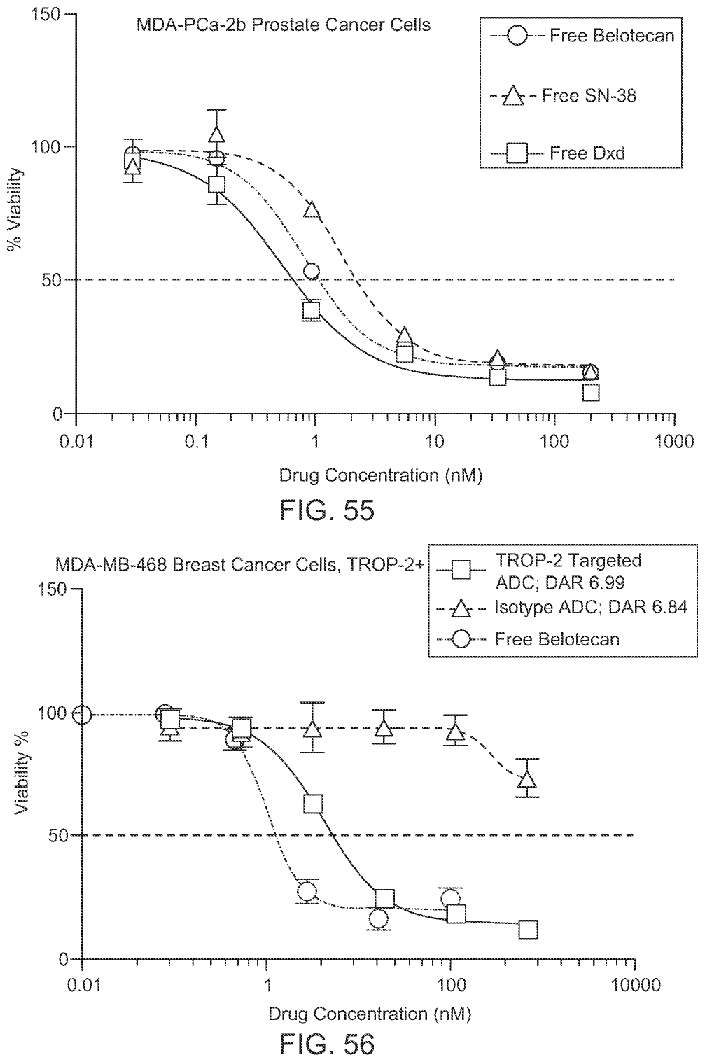

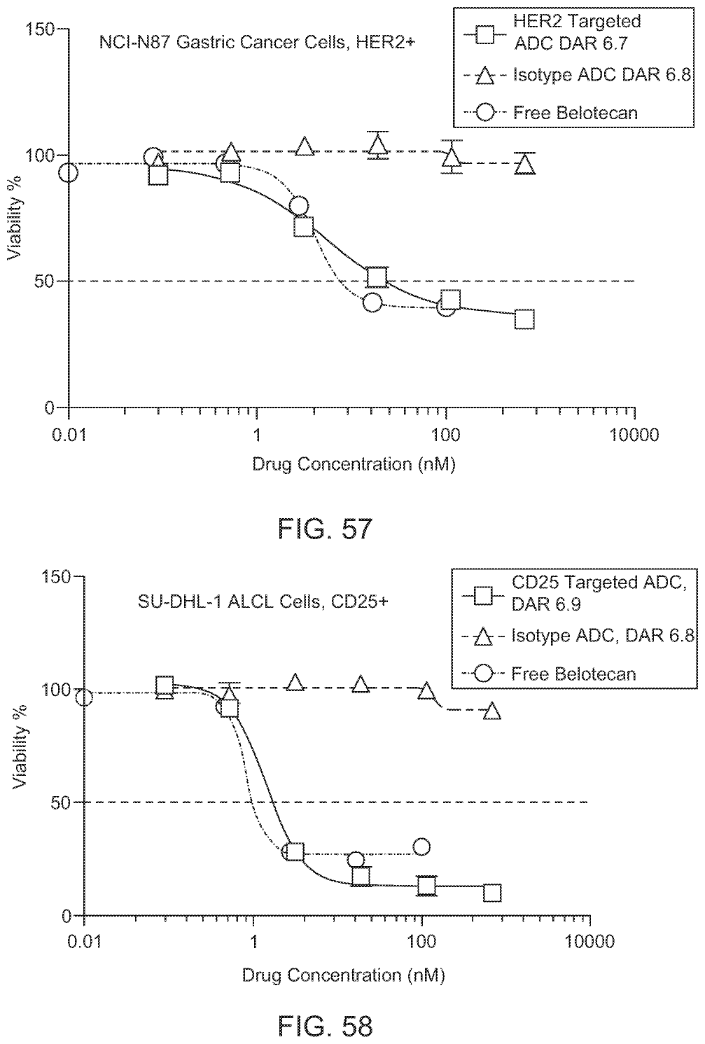

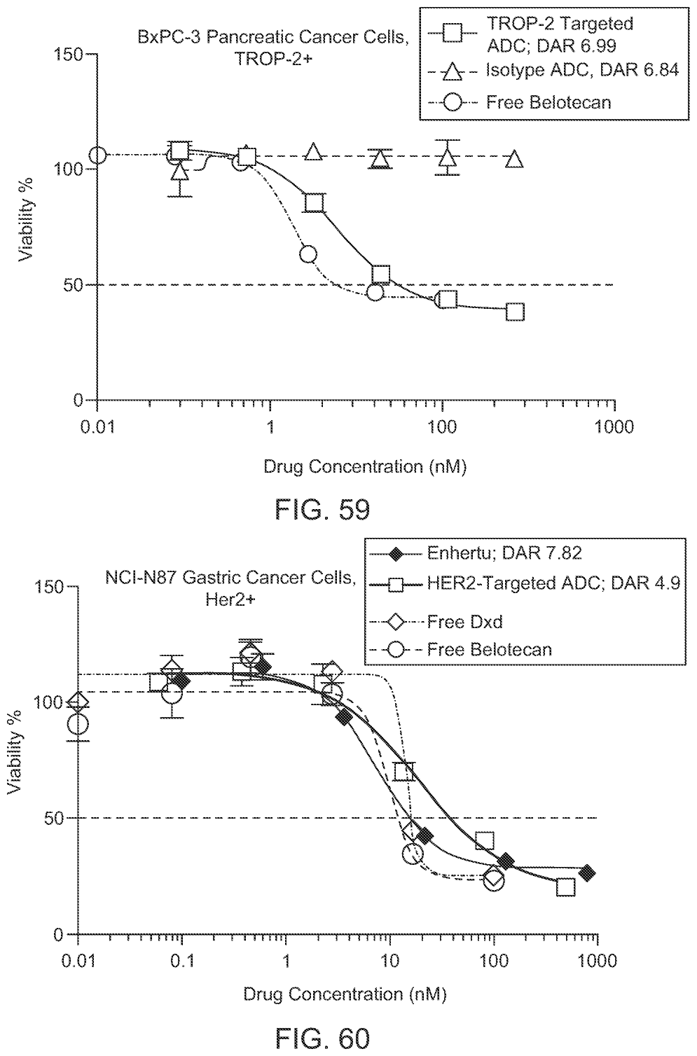

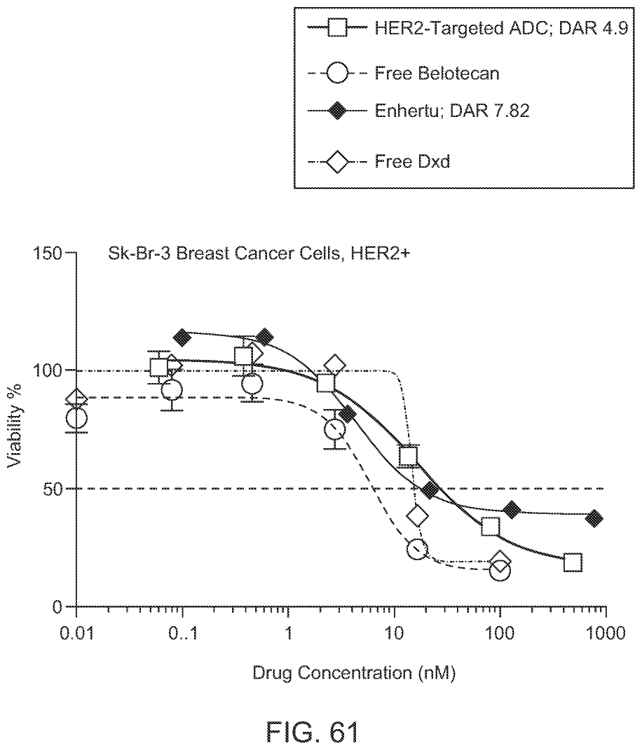

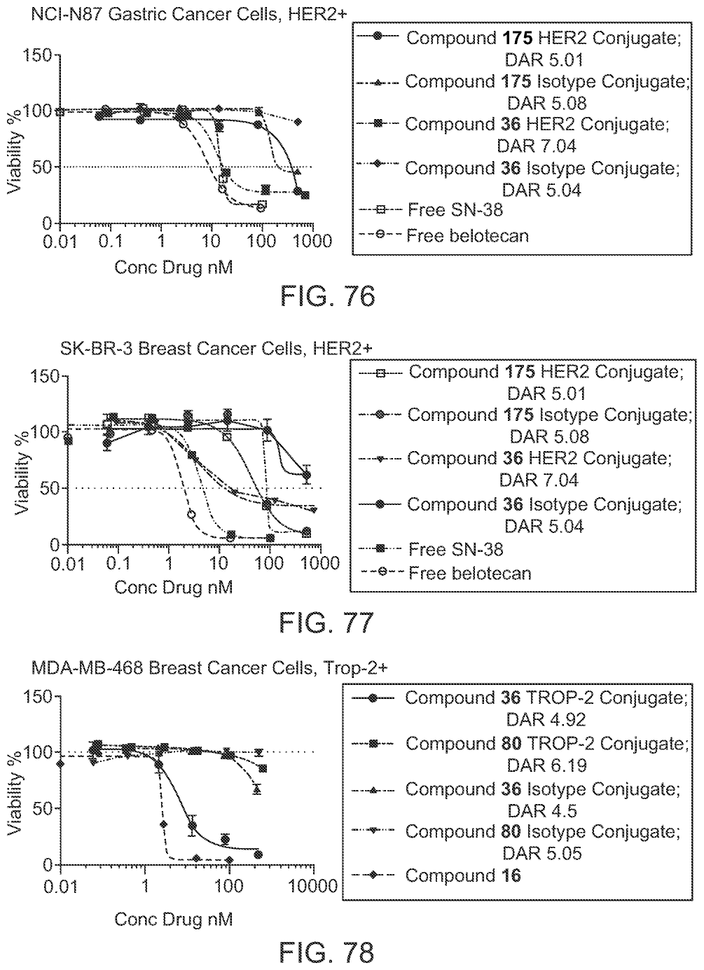

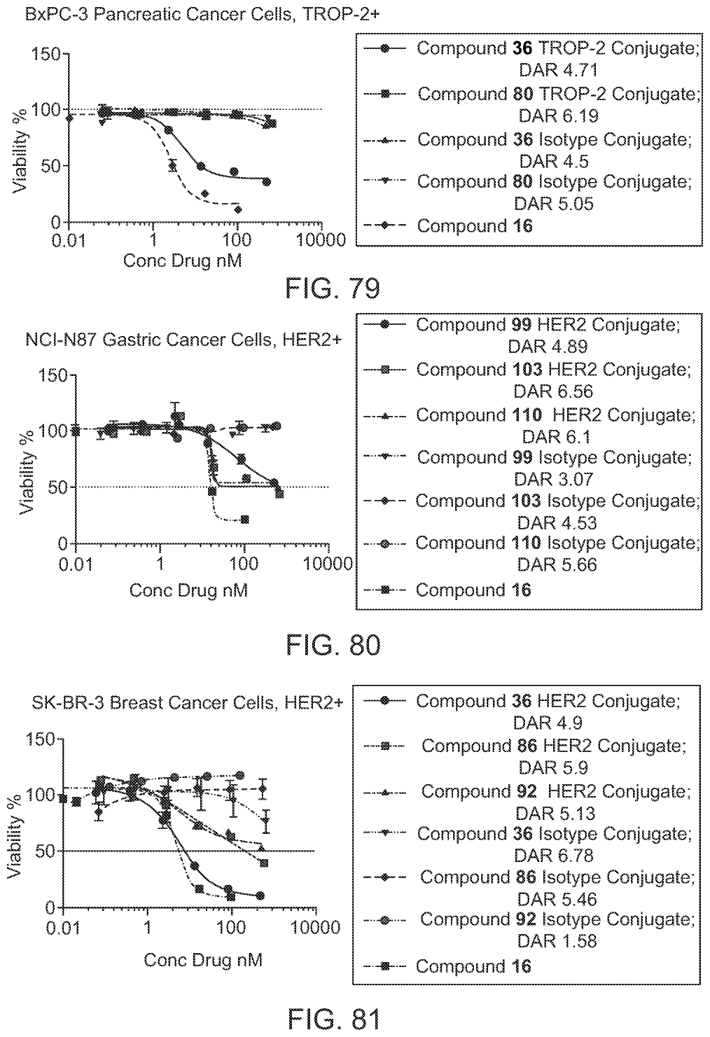

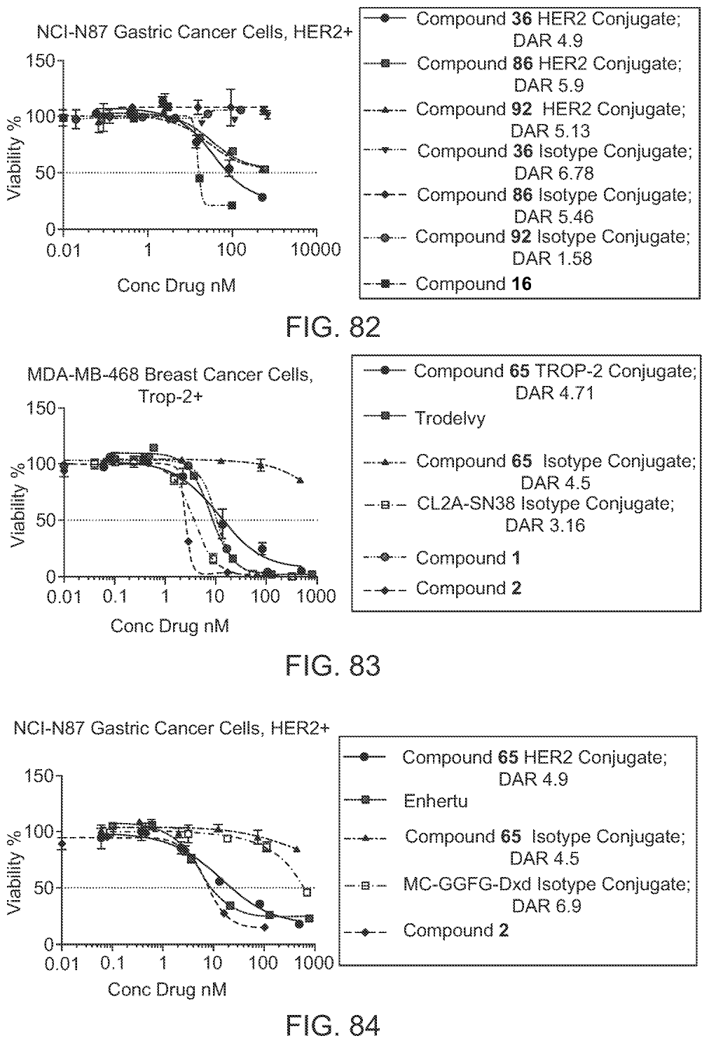

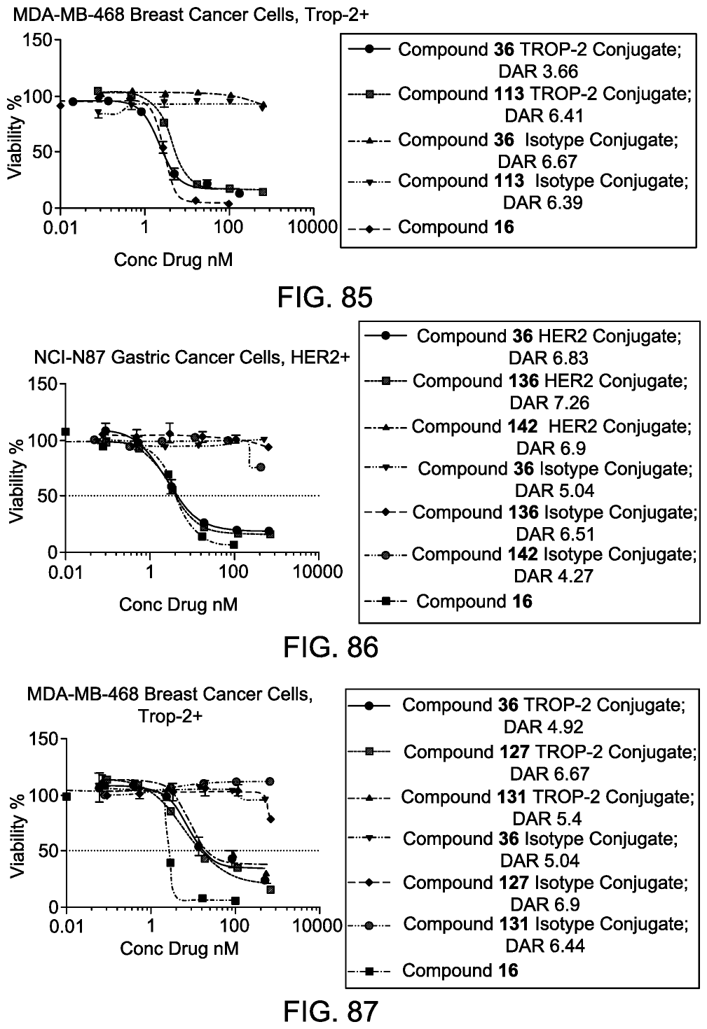

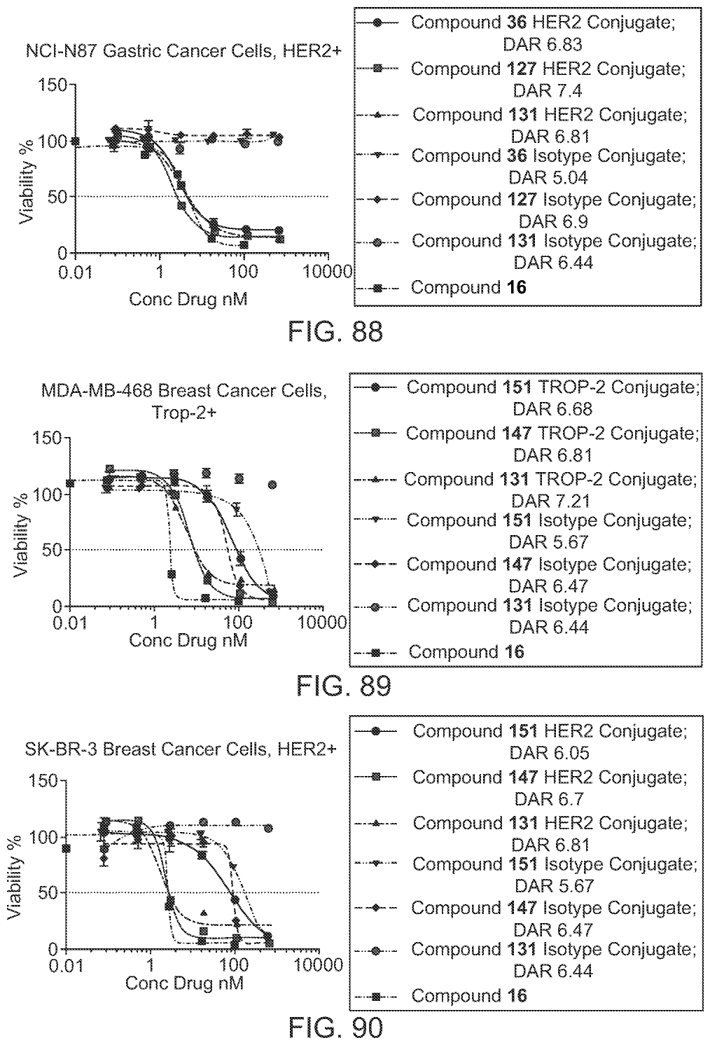

Cell lines were plated in 96-well plates (Costar 3610) at a density of 5×104 cells/well in 100 μL of growth media. The next day, cells were treated with 20 μL of test compounds serially-diluted in media. After incubation at 37° C. with 5% CO₂ for 5 days, viability was measured using the Promega CellTiter Glo® reagent according to the manufacturer's recommendations. GI50 curves were calculated in GraphPad Prism normalized to the payload concentration. Graphs of the cytotoxicity assays (% viability vs. drug concentration (nM)) are shown in FIGS. 51-61 and FIGS. 76-90.

### Example 7

### Rat Pharmacokinetic (PK) Study

Male Sprague-Dawley rats (3 per group) were dosed intravenously with a single 0.9 mg/kg bolus of test article. K2EDTA-stabilized plasma was collected at 1 h, 8 h and 24 h, and 2, 4, 6, 8, 10, and 14 days post-dose.

### PK Sample Analysis

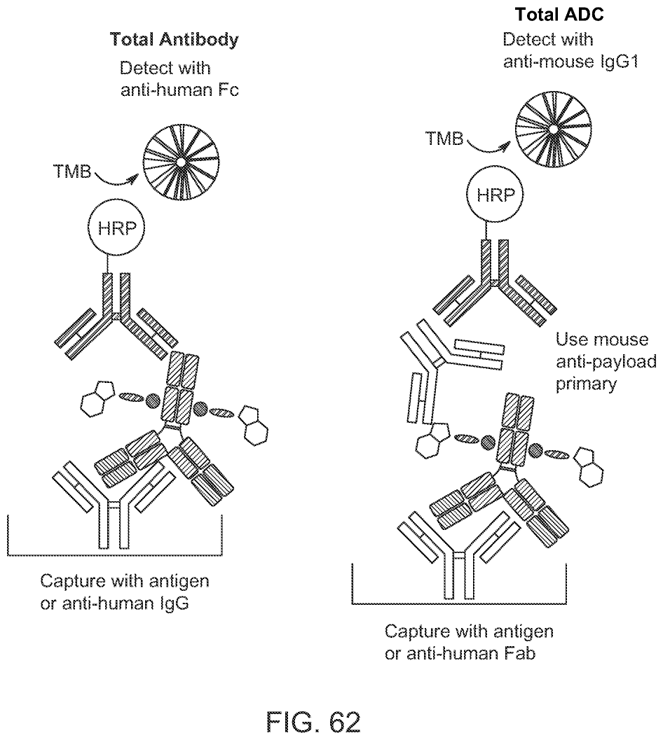

Total antibody and total ADC concentrations were quantified by ELISA as diagrammed in FIG. 62. For total antibody, conjugates were captured with an anti-human IgG-specific antibody and detected with an HRP-conjugated anti-human Fc-specific antibody. For total ADC, conjugates were captured with an anti-human Fab-specific antibody and detected with a mouse anti-payload primary antibody, followed by an HRP-conjugated anti-mouse IgG-subclass 1-specific secondary antibody. Bound secondary antibody was detected using Ultra TMB One-Step ELISA substrate (Thermo Fisher). After quenching the reaction with sulfuric acid, signals were read by taking the absorbance at 450 nm on a Molecular Devices Spectra Max M5 plate reader equipped with SoftMax Pro software. Data were analyzed using GraphPad Prism and Microsoft Excel software.

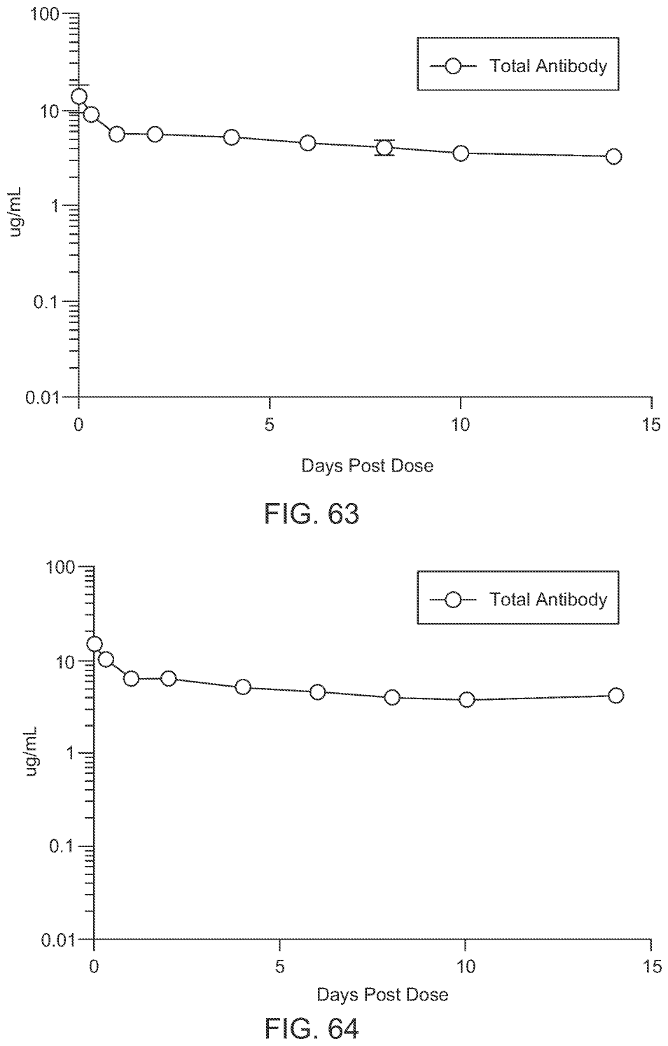

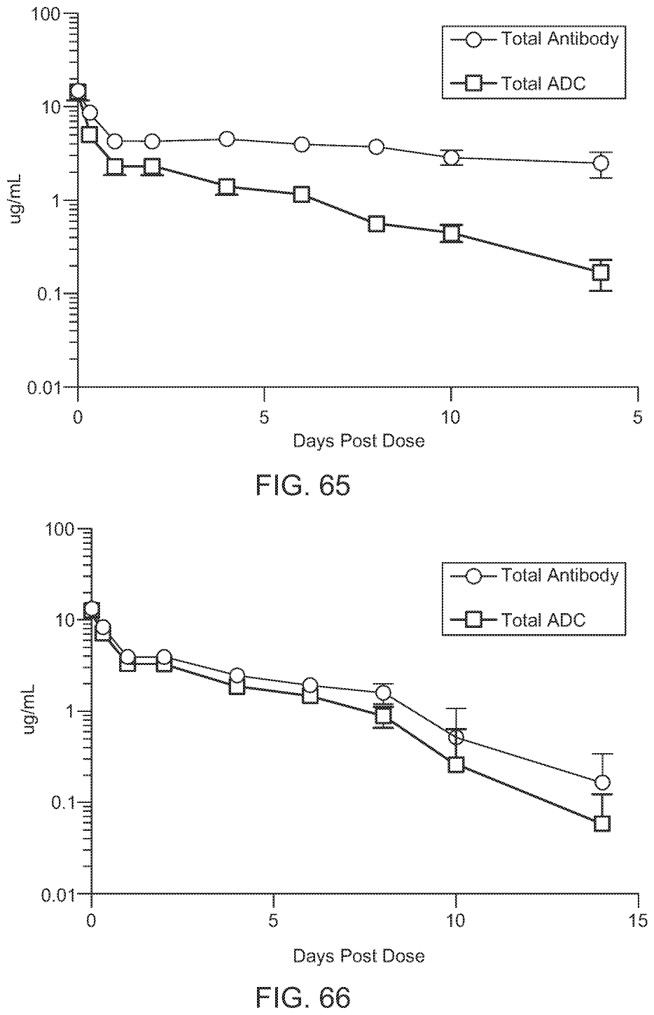

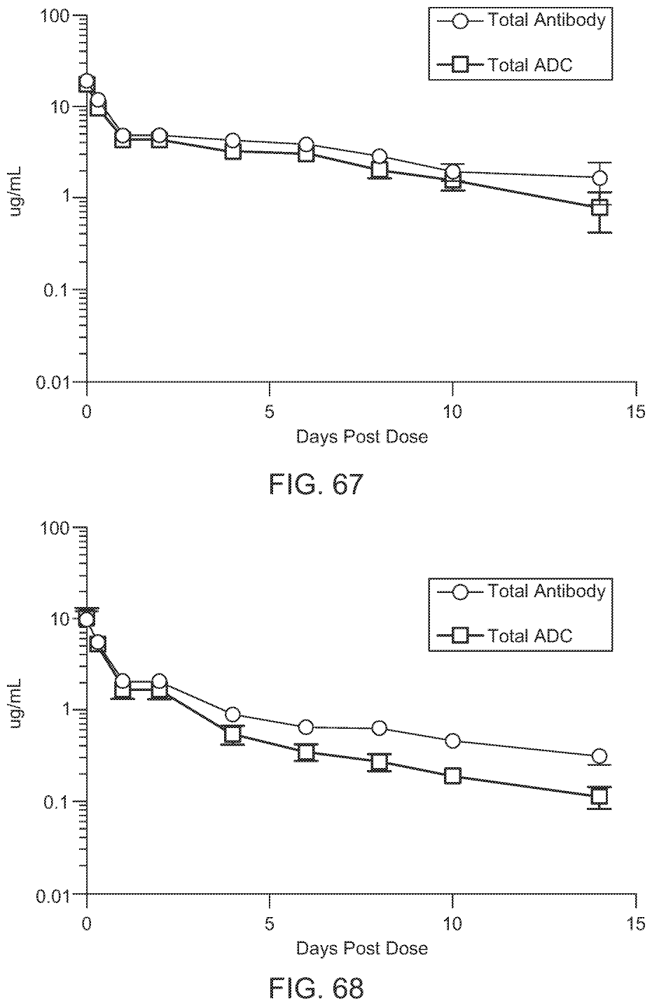

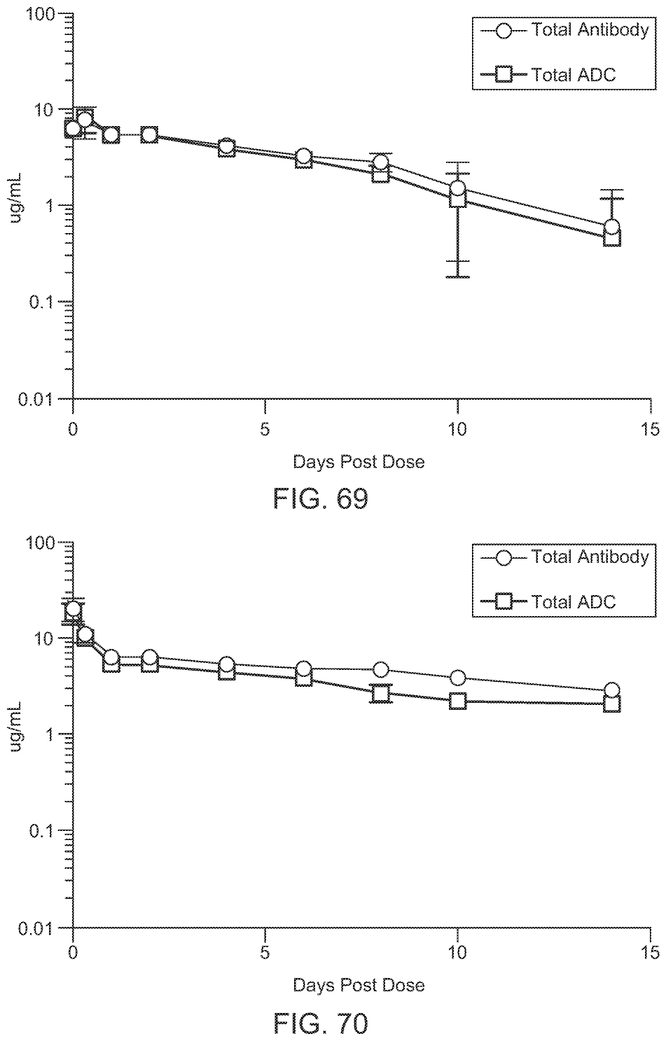

The results of the PK sample analysis are shown in FIGS. 63-70. FIG. 63 shows a graph of concentration (μg/mL) vs. days post dose following a 0.9 mg/kg dose of trastuzumab antibody. FIG. 64 shows a graph of concentration (μg/mL) vs. days post dose following a 0.9 mg/kg dose of polatuzumab antibody. FIG. 65 shows a graph of concentration (μg/mL) vs. days post dose following a 0.9 mg/kg dose of a conventional HER2 topoisomerase inhibitor conjugated ADC bearing a protease-cleavable linker. FIG. 66 shows a graph of concentration (μg/mL) vs. days post dose following a 0.9 mg/kg dose of CH1-3/CT-tagged trastuzumab conjugated to construct 32. FIG. 67 shows a graph of concentration (μg/mL) vs. days post dose following a 0.9 mg/kg dose of CH1-3/CT-tagged trastuzumab conjugated to construct 36. FIG. 68 shows a graph of concentration (μg/mL) vs. days post dose following a 0.9 mg/kg dose of CT-tagged polatuzumab conjugated to construct 15. FIG. 69 shows a graph of concentration (μg/mL) vs. days post dose following a 0.9 mg/kg dose of CH2-3-tagged polatuzumab conjugated to construct 15. FIG. 70 shows a graph of concentration (μg/mL) vs. days post dose following a 0.9 mg/kg dose of CH₁-2-tagged polatuzumab conjugated to construct 15.

### Example 8

### Xenograft Studies

### Study 1: NCI-H292 Xenograft

Methods: Female SCID Beige mice (8/group) were inoculated subcutaneously with 5 million NCI-H292 cells in PBS. Treatment began when the tumors reached an average of 121 mm³ (Day 1). For the Study 1 treatment, animals were dosed intravenously with vehicle alone, Trodelvy, DS-1062, or with conjugate 3485, a TROP-2 targeted ADC including two tag sites conjugated to compound 36 (with a DAR of 6.85). ADCs were dosed at either 10 mg/kg on Days 0, 7, and 21 (Trodelvy) or at 6 mg/kg on Days 0 and 21 (DS-1062 and conjugate 3485). The animals were monitored twice weekly for body weight and tumor size. Animals were euthanized when tumors reached 2000 mm³ or body weight loss exceeded 15%.

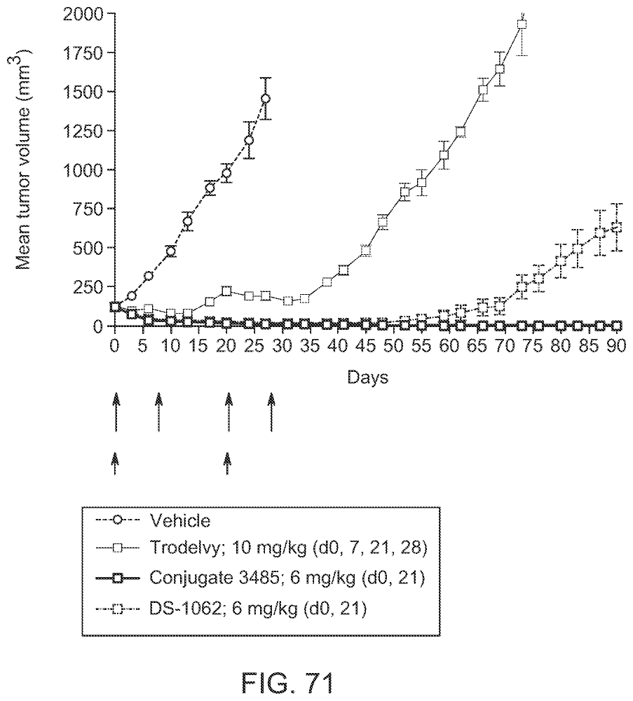

Results are shown in FIG. 71, which shows a graph of mean tumor volume (mm³) vs. days and indicates in vivo efficacy against the NCI-H292 xenograft of TROP-2 targeted ADCs carrying topoisomerase inhibitor payloads. n=8 mice/group; dosing is indicated by arrows.

### Study 2: NCI-H292 Xenograft

Methods: Female SCID Beige mice (7/group) were inoculated subcutaneously with 5 million NCI-H292 cells in PBS. Treatment began when the tumors reached an average of 121 mm³ (Day 1). For the Study 2 treatment, animals were dosed intravenously with vehicle alone, DS-1062, or with conjugates 3485, 3789, or 3790, and TROP-2 targeted ADCs including two tag sites conjugates to compounds 36, 127, or 131, respectively. The animals were monitored twice weekly for body weight and tumor size. Animals were euthanized when tumors reached 2000 mm³ or body weight loss exceeded 15%.

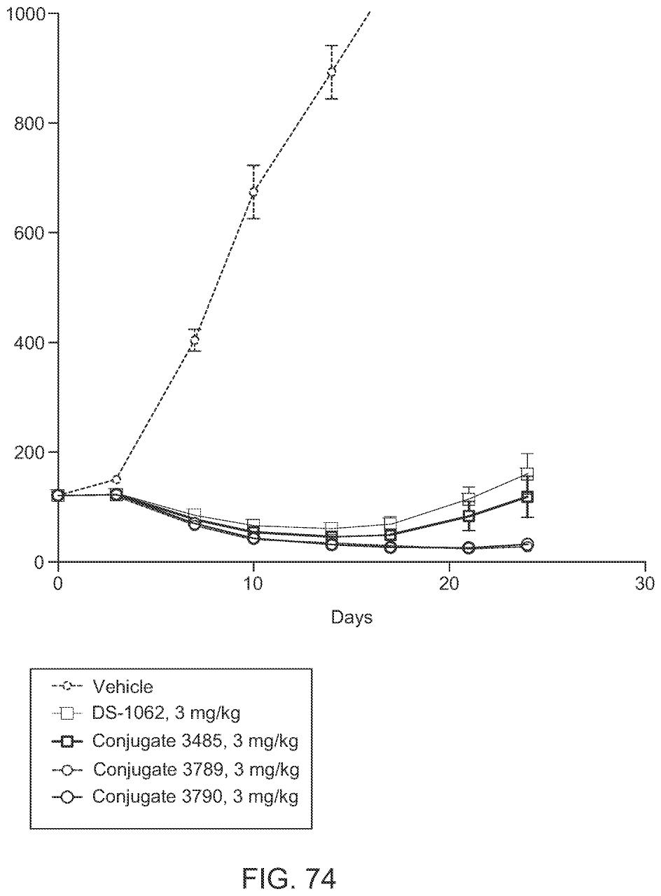

Results are shown in FIG. 74, which shows a graph of mean tumor volume (mm³) vs. days and indicates in vivo efficacy against an NCI-H292 xenograft of TROP-2 targeted ADCs carrying topoisomerase inhibitor payloads. n=7 mice/group. A single i.v. dose was delivered on Day 0.

### Study 3: NCI-H1781 Xenograft

Methods: Female BALB/c nude mice (5/group) were inoculated subcutaneously with 20 million NCI-H1781 cells in PBS. Treatment began when the tumors reached an average of 222 mm³ (Day 1). Animals were dosed intravenously with vehicle alone, a nectin-4 Compound 36 conjugate with a DAR of 6.8, or a nectin-4 mc-GGFG-Dxd conjugate with a DAR of 3.7. ADCs were dosed intravenously at 5 mg/kg on Days 0 and 7. The animals were monitored twice weekly for body weight and tumor size. Animals were euthanized when tumors reached 2000 mm³ or body weight loss exceeded 15%.

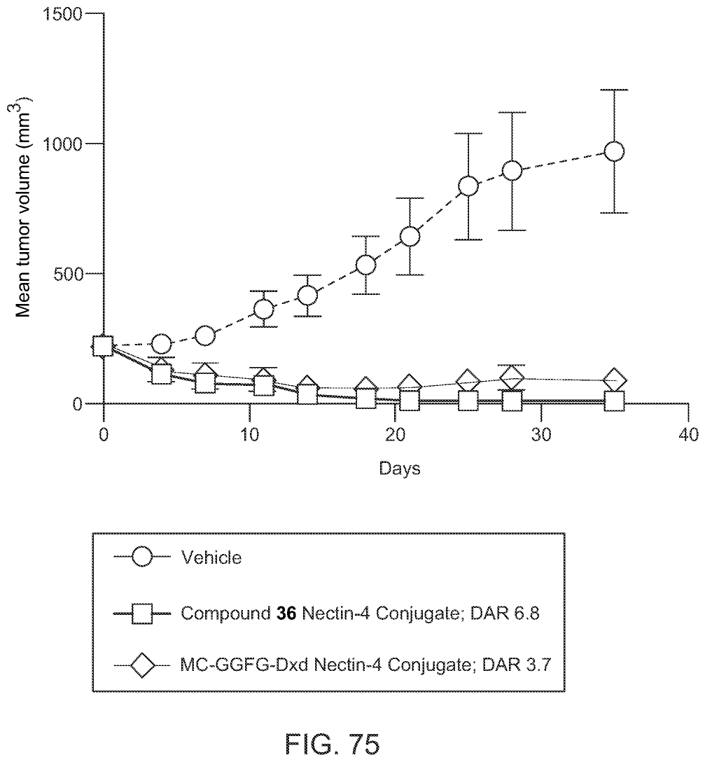

Results are shown in FIG. 75, which shows a graph of in vivo efficacy against the NCI-H1781 xenograft of nectin-4 targeted ADCs carrying topoisomerase inhibitor payloads. n=5 mice/group. A 5 mg/kg dose was delivered i.v. on Days 0 and 7.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A conjugate of formula (I): 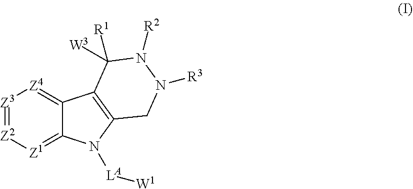
wherein:
Z¹, Z², Z³ and Z⁴ are each independently selected from CR⁴, N and C-L^{B}-W², wherein at least one Z¹, Z², Z³ and Z⁴ is C-L^{B}-W²;
R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L^{A} is a first linker;
L^{B} is a second linker;
W¹ is a first drug;
W² is a second drug; and
W³ is a polypeptide.

2. The conjugate of claim 1, wherein Z¹ is CR⁴.

3. The conjugate of claim 1, wherein Z¹ is N.

4. The conjugate of claim 1, wherein Z³ is C-L^{B}-W².

5. The conjugate of any of claims 1-4, wherein L^{A} comprises:
-(T¹-V¹)ₐ-(T²-V²)_{b}-(T³-V³)_{c}-(T⁴-V⁴)_{d}-(T⁵-V⁵)ₑ-(T⁶-V⁶)_{f}-,
wherein
a, b, c, d, e and f are each independently 0 or 1;
T¹, T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

6. The conjugate of claim 5, wherein:
T¹ is selected from a (C₁-C₁₂)alkyl and a substituted (C₁-C₁₂)alkyl;
T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PHP, an acetal group, a hydrazine, and an ester; and
V¹, V², V¹, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂—, and —P(O)OH—;
wherein:
(PEG)ₙ is 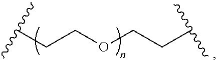
where n is an integer from 1 to 30;
EDA is an ethylene diamine moiety having the following structure: 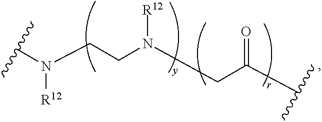
where y is an integer from 1 to 6 and r is 0 or 1;
4-amino-piperidine (4AP) is 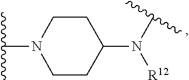
and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring.

7. The conjugate of any of claims 5-6, wherein T¹, T², T³, T⁴, T⁵ and T⁶ are each optionally substituted with a glycoside.

8. The conjugate of any of claims 5-6, wherein MABO, MABC, PABO, PABC, PAB, PABA, PAP and PHP are each optionally substituted with a glycoside.

9. The conjugate of any of claims 7-8, wherein the glycoside is selected from a glucuronide, a galactoside, a glucoside, a mannoside, a fucoside, O-GlcNAc, and O-GalNAc.

10. The conjugate of any of claims 5-9,
wherein:
T¹ is (C₁-C₁₂)alkyl and V¹ is —CO—;
T² is AA and V² is absent;
T³ is PABC and V³ is absent; and
d, e and f are each 0; or
wherein:
T¹ is (C₁-C₁₂)alkyl and V¹ is —CONH—;
T² is (PEG)ₙ and V² is —CO—;
T³ is AA and V³ is absent;
T⁴ is PABC and V⁴ is absent; and
e and f are each 0; or
wherein:
T¹ is (C₁-C₁₂)alkyl and V¹ is —CO—;
T² is an amino acid analog and V² is —NH—;
T³ is (PEG)ₙ and V³ is —CO—;
T⁴ is AA and V⁴ is absent;
T⁵ is PABC and V⁵ is absent; and
f is 0; or
wherein:
T¹ is (C₁-C₁₂)alkyl and V¹ is —CONH—;
T² is (PEG)ₙ and V² is —CO—;
T³ is AA and V³ is absent;
T⁴ is PABC and V⁴ is absent; and
e and f are each 0; or
wherein:
T¹ is (C₁-C₁₂)alkyl and V¹ is —CONH—;
T² is substituted (C₁-C₁₂)alkyl and V² is —CO—;
T³ is AA and V³ is absent;
T⁴ is PABC and V⁴ is absent; and
e and f are each 0; or
wherein:
T¹ is (C₁-C₁₂)alkyl and V¹ is —CONH—;
T² is (PEG)ₙ and V² is —CO—;
T³ is AA and V³ is absent;
T⁴ is PABA and V⁴ is —CO—;
T⁵ is (C₁-C₁₂)alkyl and V⁵ is absent; and
f is 0; or
wherein:
T¹ is (C₁-C₁₂)alkyl and V¹ is —CO—;
T² is 4AP and V² is —CO—;
T³ is (C₁-C₁₂)alkyl and V³ is —CO—;
T⁴ is AA and V⁴ is absent;
T⁵ is PABC and V⁵ is absent; and
f is 0; or
wherein:
T¹ is (C₁-C₁₂)alkyl and V¹ is —CO—;
T² is 4AP and V² is —CO—;
T³ is (C₁-C₁₂)alkyl and V³ is —O—;
T⁴ is (C₁-C₁₂)alkyl and V⁴ is —CO—;
T⁵ is AA and V⁵ is absent; and
T⁶ is PABC and V⁶ is absent; or
wherein:
T¹ is (C₁-C₁₂)alkyl and V¹ is —CO—;
T² is an amino acid analog and V² is absent;
T³ is AA and V³ is absent;
T⁴ is PABC and V⁴ is absent; and
e and f are each 0; or
wherein:
T¹ is (C₁-C₁₂)alkyl and V¹ is —CONH—;
T² is (PEG)ₙ and V² is —CONH—;
T³ is substituted (C₁-C₁₂)alkyl and V³ is —CO—;
T⁴ is AA and V⁴ is absent;
T⁵ is PABC and V⁵ is absent; and
f is 0; or
wherein:
T¹ is (C₁-C₁₂)alkyl and V¹ is —CO—;
T² is an AA and V² is —NH—;
T³ is (PEG)ₙ and V³ is —CO—;
T⁴ is AA and V⁴ is absent;
T⁵ is PABC and V⁵ is absent; and
f is 0; or
wherein:
T¹ is (C₁-C₁₂)alkyl and V¹ is —CONH—;
T² is (PEG)ₙ and V² is —CO—;
T³ is AA and V³ is absent;
T⁴ is PAP and V⁴ is —C(O)O—; and
e and f are each 0; or
wherein:
T¹ is (C₁-C₁₂)alkyl and V¹ is —CONH—;
T² is substituted (C₁-C₁₂)alkyl and V² is —CO—;
T³ is AA and V³ is absent;
T⁴ is PABC and V⁴ is absent; and
e and f are each 0; or
wherein:
T¹ is (C₁-C₁₂)alkyl and V¹ is —CONH—;
T² is substituted (C₁-C₁₂)alkyl and V² is —CO—;
T³ is PABC and V³ is absent; and
d, e and f are each 0.

11. The conjugate of any of claims 1-10, wherein L^{B} comprises:
-(T⁷-V⁷)_{g}-(T⁸-V⁸)ₕ-(T⁹-V⁹)ᵢ-(T¹⁰-V¹⁰)ⱼ-(T¹¹-V¹¹)ₖ-(T¹²-V¹²)ₗ-(T¹³-V¹³)ₘ,
wherein
g, h, i, j, k, l and m are each independently 0 or 1;
T⁷, T⁸, T⁹, T¹⁰, T¹¹, T¹² and T¹³ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V⁷, V⁸, V⁹, V¹⁰, V¹¹, V¹² and V¹³ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

12. The conjugate of claim 11, wherein T⁷, T⁸, T⁹, T¹⁰, T¹¹, T¹² and T¹³ are each optionally substituted with a glycoside.

13. The conjugate of claim 11, wherein MABO, MABC, PABO, PABC, PAB, PABA, PAP and PHP are each optionally substituted with a glycoside.

14. The conjugate of any of claims 12-13, wherein the glycoside is selected from a glucuronide, a galactoside, a glucoside, a mannoside, a fucoside, O-GlcNAc, and O-GalNAc.

15. The conjugate of any of claims 11-14,
wherein:
T⁷ is absent and V⁷ is —NHCO—;
T⁸ is (C₁-C₁₂)alkyl and V⁸ is —CO—;
T⁹ is AA and V⁹ is absent;
T¹⁰ is PABC and V¹⁰ is absent; and
k, l and m are each 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is AA and V¹⁰ is absent; and
T¹¹ is PABC and V¹¹ is absent; and
l and m are each 0; or
wherein:
T⁷ is absent and V⁷ is —NHCO—;
T⁸ is (C₁-C₁₂)alkyl and V⁸ is —CO—;
T⁹ is an amino acid analog and V⁹ is —NH—;
T¹⁰ is (PEG)ₙ and V¹⁰ is —CO—;
T¹¹ is AA and V¹¹ is absent;
T¹² is PABC and V¹² is absent; and
m is 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is AA and V¹⁰ is absent;
T¹¹ is PABC and V¹¹ is absent; and
l and m are each 0; or
wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is AA and V¹⁰ is absent;
T¹¹ is PABC and V¹¹ is absent; and
l and m are each 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is AA and V¹⁰ is absent;
T¹¹ is PABA and V¹¹ is —CO—;
T¹² is (C₁-C₁₂)alkyl and V¹² is absent; and
m is 0; or
wherein:
T⁷ is absent and V⁷ is —NHCO—;
T⁸ is (C₁-C₁₂)alkyl and V⁸ is —CO—;
T⁹ is 4AP and V⁹ is —CO—;
T¹⁰ is (C₁-C₁₂)alkyl and V¹⁰ is —CO—;
T¹¹ is AA and V¹¹ is absent;
T¹² is PABC and V¹² is absent; and
m is 0; or
wherein:
T⁷ is absent and V⁷ is —NHCO—;
T⁸ is (C₁-C₁₂)alkyl and V⁸ is —CO—;
T⁹ is 4AP and V⁹ is —CO—;
T¹⁰ is (C₁-C₁₂)alkyl and V¹⁰ is —O—;
T¹¹ is (C₁-C₁₂)alkyl and V¹¹ is —CO—;
T¹² is AA and V¹² is absent; and
T¹³ PABC and V¹³ is absent; or
wherein:
T⁷ is absent and V⁷ is —NHCO—;
T⁸ is (C₁-C₁₂)alkyl and V⁸ is —CO—;
T⁹ is an amino acid analog and V⁹ is absent;
T¹⁰ is AA and V¹⁰ is absent;
T¹¹ is PABC and V¹¹ is absent; and
l and m are each 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CONH—;
T¹⁰ is substituted (C₁-C₁₂)alkyl and V¹⁰ is —CO—;
T¹¹ is AA and V¹¹ is absent;
T¹² is PABC and V¹² is absent; and
m is 0; or
wherein:
T⁷ is absent and V⁷ is —NHCO—;
T⁸ is (C₁-C₁₂)alkyl and V⁸ is —CO—;
T⁹ is AA and V⁹ is —NH—;
T¹⁰ is (PEG)ₙ and V¹⁰ is —CO—;
T¹¹ is AA and V¹¹ is absent;
T¹² is PABC and V¹² is absent; and
m is 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is AA and V¹⁰ is absent;
T¹¹ is PAP and V¹¹ is —C(O)O—; and
l and m are each 0; or
wherein:
T⁷ is absent and V⁷ is —NHCO—;
T⁸ is (C₁-C₁₂)alkyl and V⁸ is —CO—;
T⁹ is AA and V⁹ is absent;
T¹⁰ is PABC and V¹⁰ is absent;
T¹¹ is PAP and V¹¹ is —C(O)O—; and
l and m are each 0; or
wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is PABC and V¹⁰ is absent; and
k, l and m are each 0; or
wherein:
T⁷ is absent and V⁷ is —NHCO—;
T⁸ is (C₁-C₁₂)alkyl and V⁸ is absent;
T⁹ is heteroaryl and V⁹ is absent;
T¹⁰ is (C₁-C₁₂)alkyl and V¹⁰ is —CONH—;
T¹¹ is (PEG)ₙ and V¹¹ is —CO—; and
l and m are each 0; or
wherein:
T⁷ is absent and V⁷ is —NHCO—;
T⁸ is (C₁-C₁₂)alkyl and V⁸ is absent;
T⁹ is heteroaryl and V⁹ is absent;
T¹⁰ is (C₁-C₁₂)alkyl and V¹⁰ is —CONH—;
T¹¹ is substituted (C₁-C₁₂)alkyl and V¹¹ is —CO—;
T¹² is AA and V¹² is absent; and
T¹³ PAB and V¹³ is absent; or
wherein:
T⁷ is absent and V⁷ is —NHCO—;
T⁸ is (C₁-C₁₂)alkyl and V⁸ is absent;
T⁹ is heteroaryl and V⁹ is absent;
T¹⁰ is (C₁-C₁₂)alkyl and V¹⁰ is —CONH—;
T¹¹ is substituted (C₁-C₁₂)alkyl and V¹¹ is —CO—;
T¹² is AA and V¹² is absent; and
T¹³ PABC and V¹³ is absent.

16. The conjugate of any of claims 1-15, wherein the conjugate is selected from:

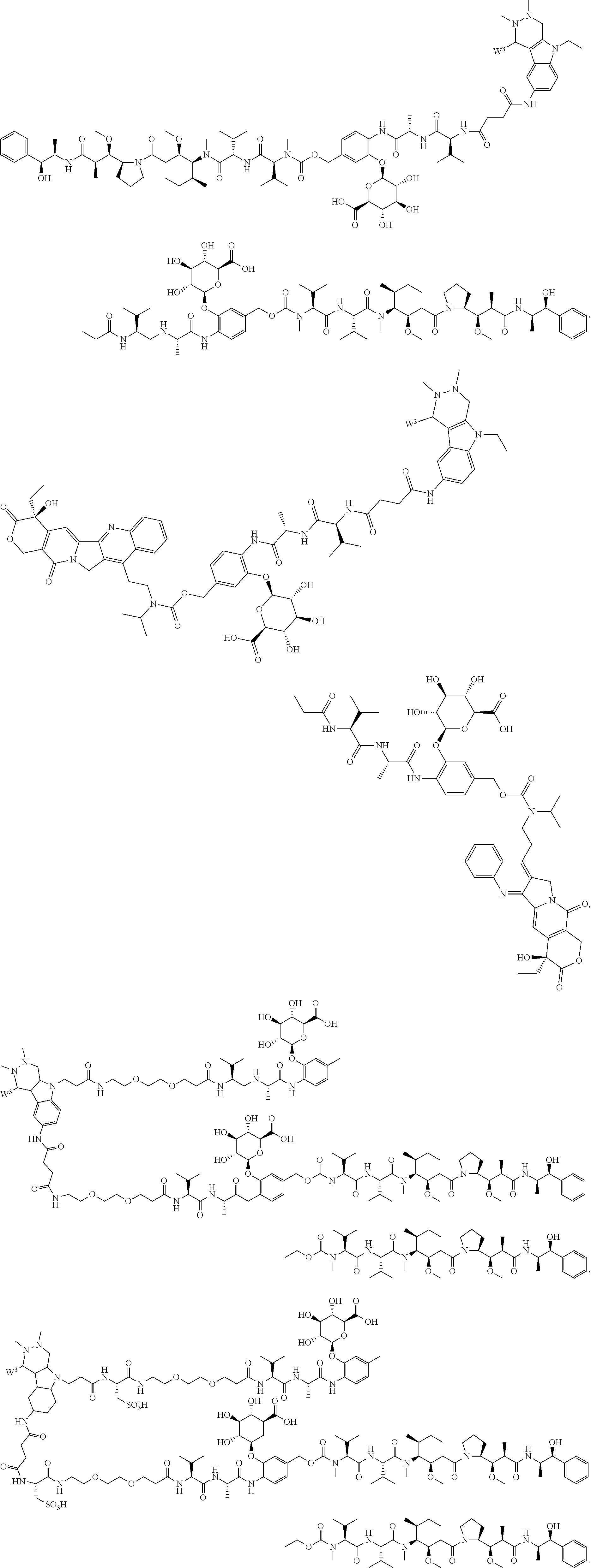

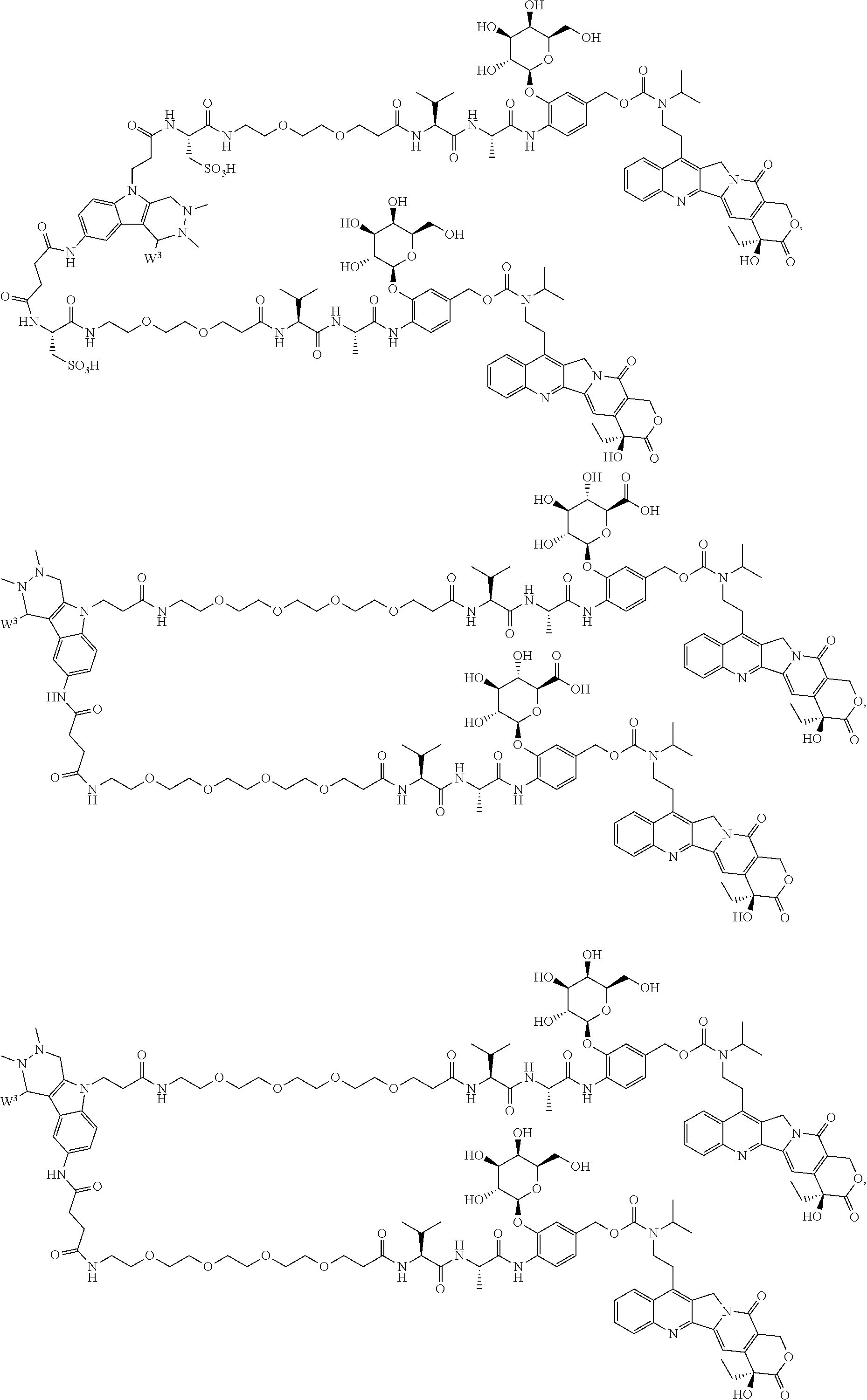

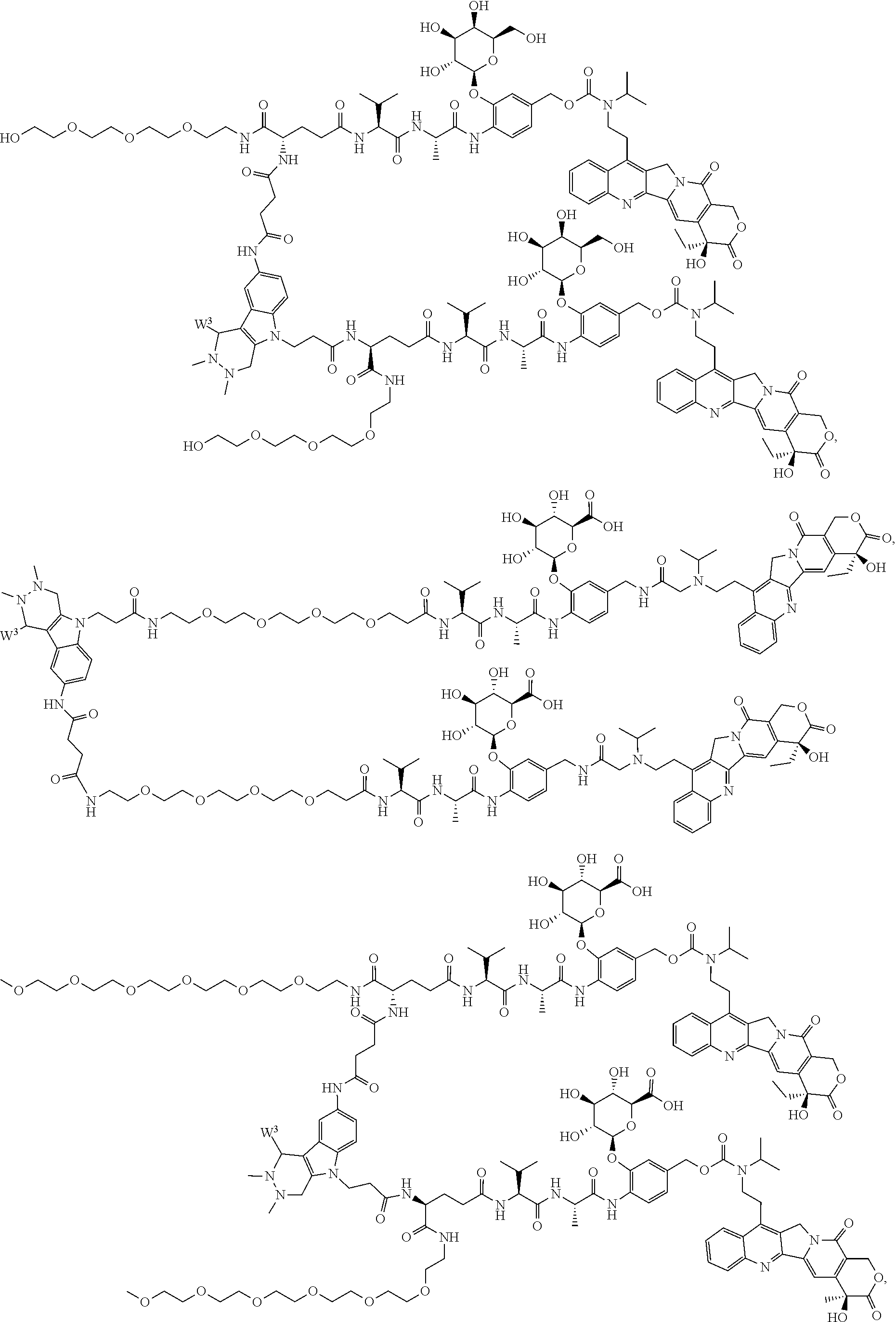

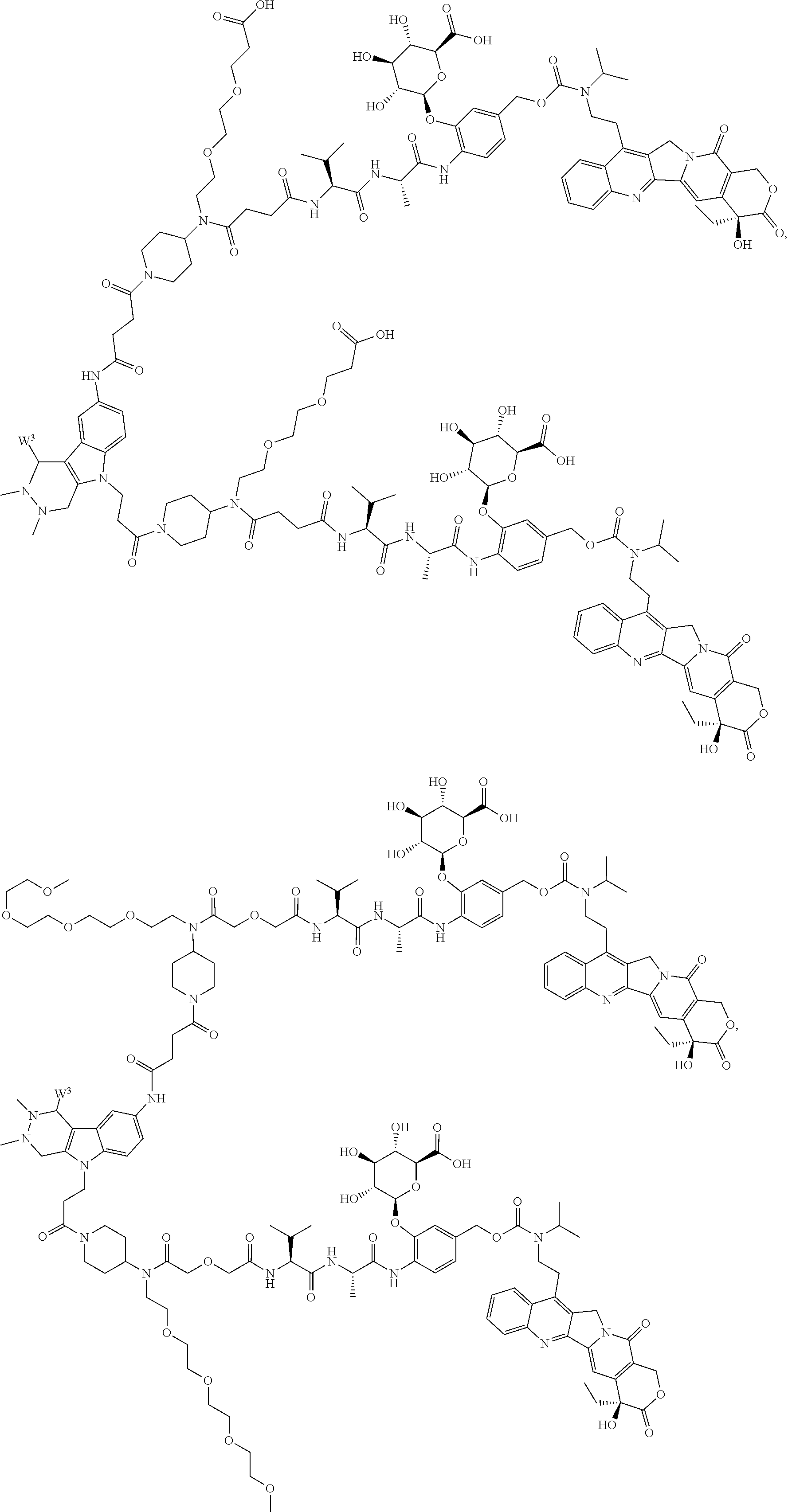

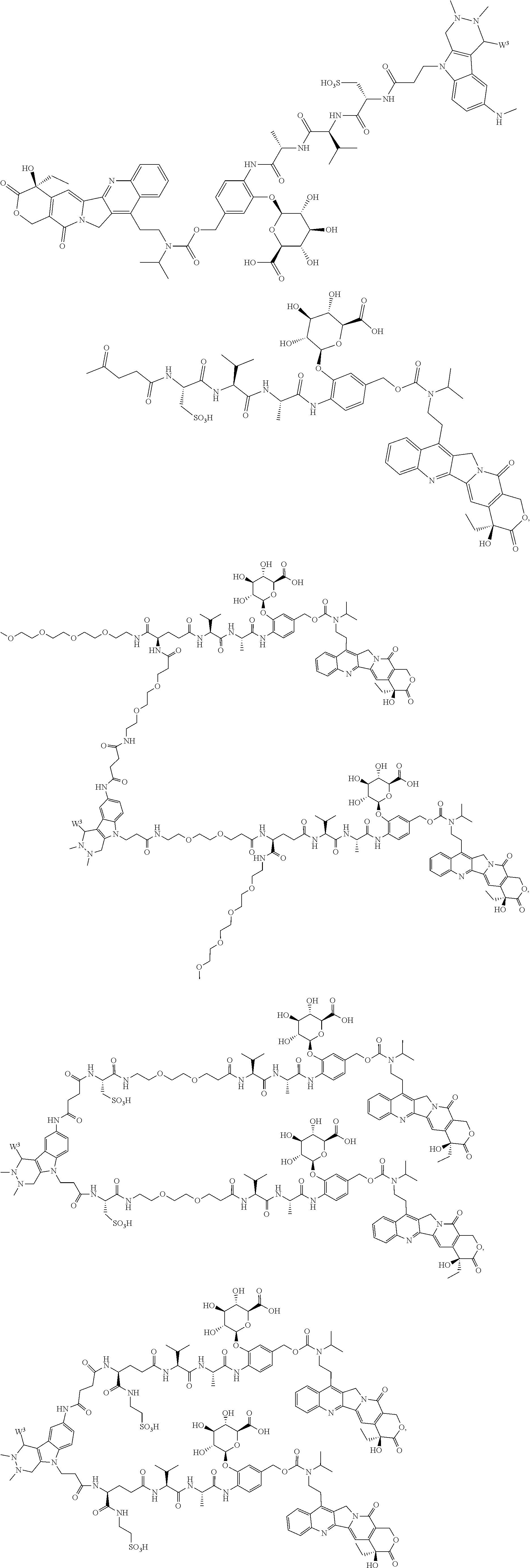

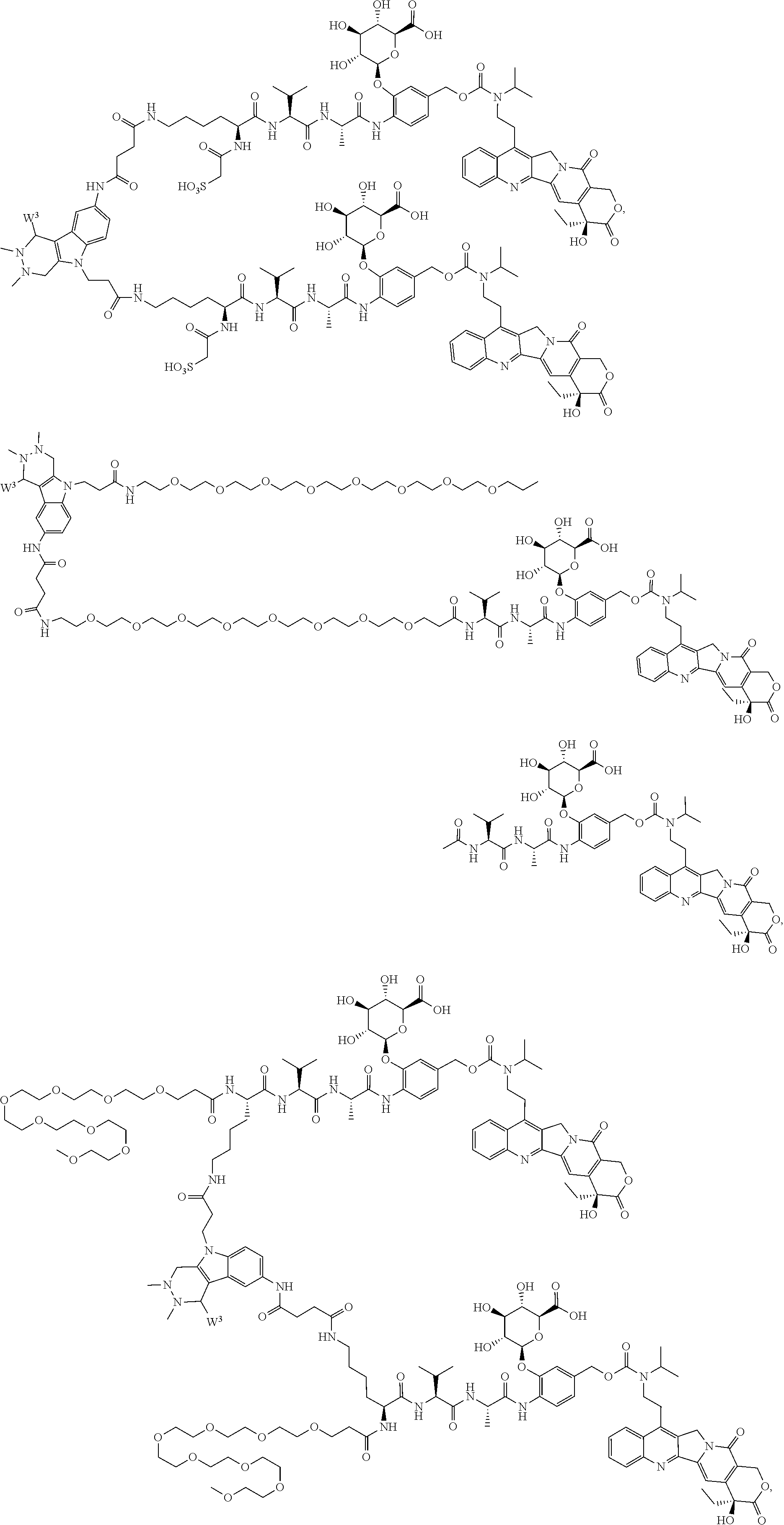

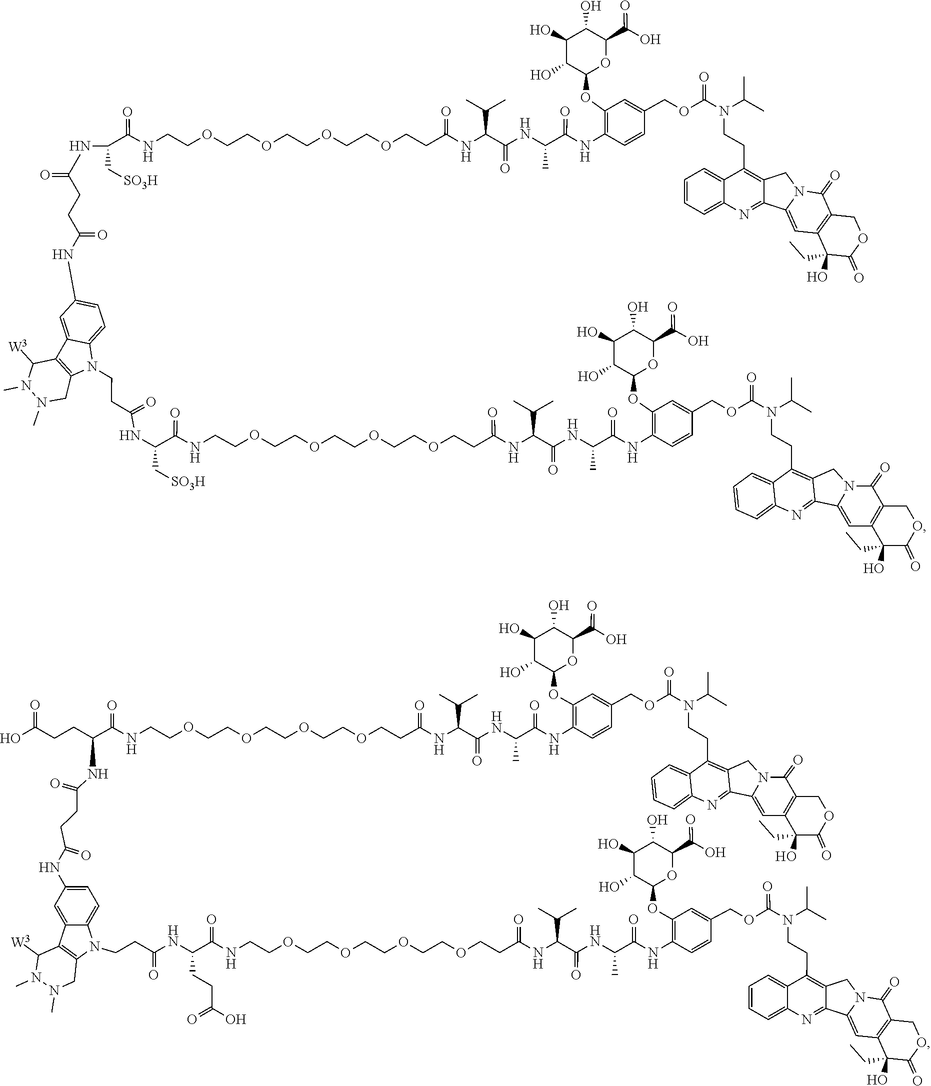

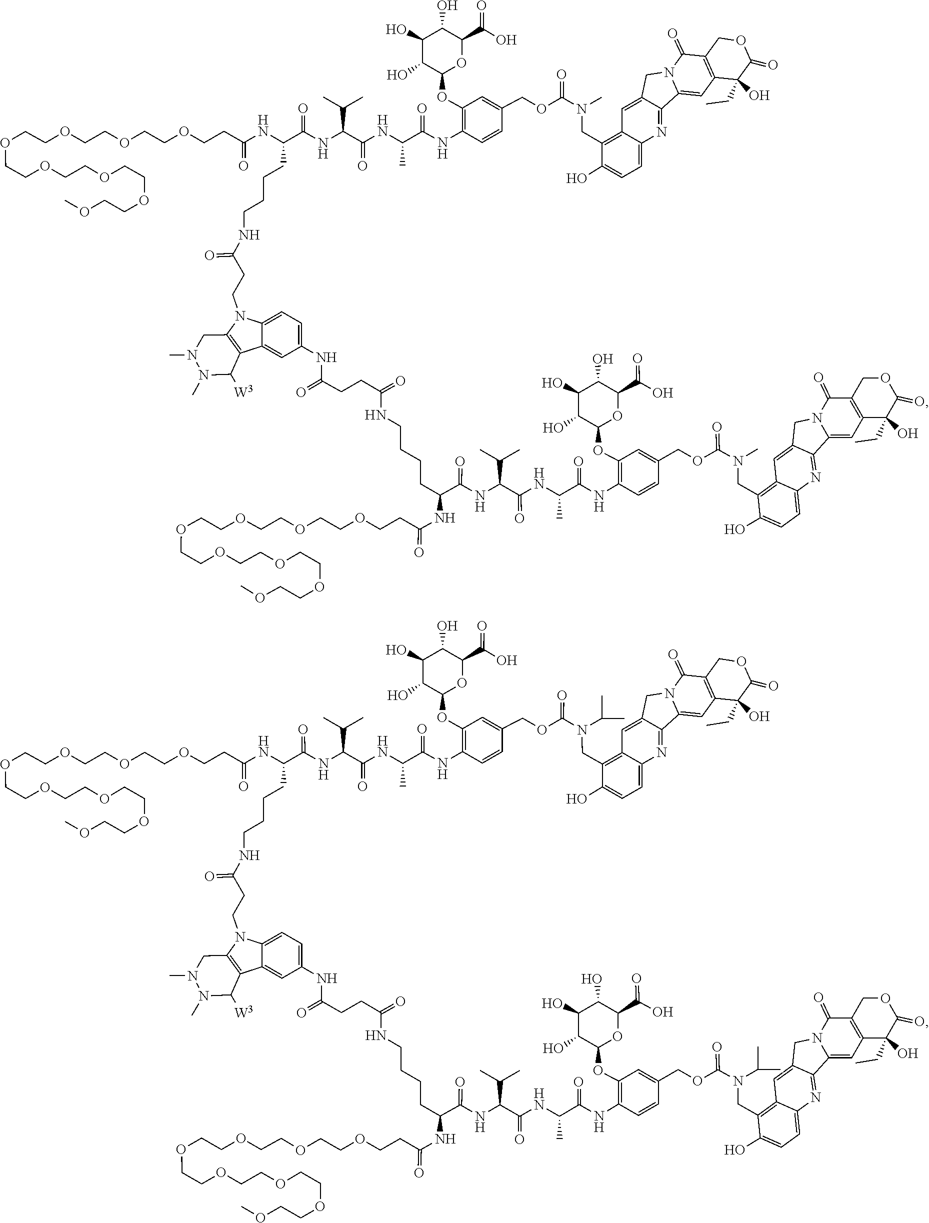

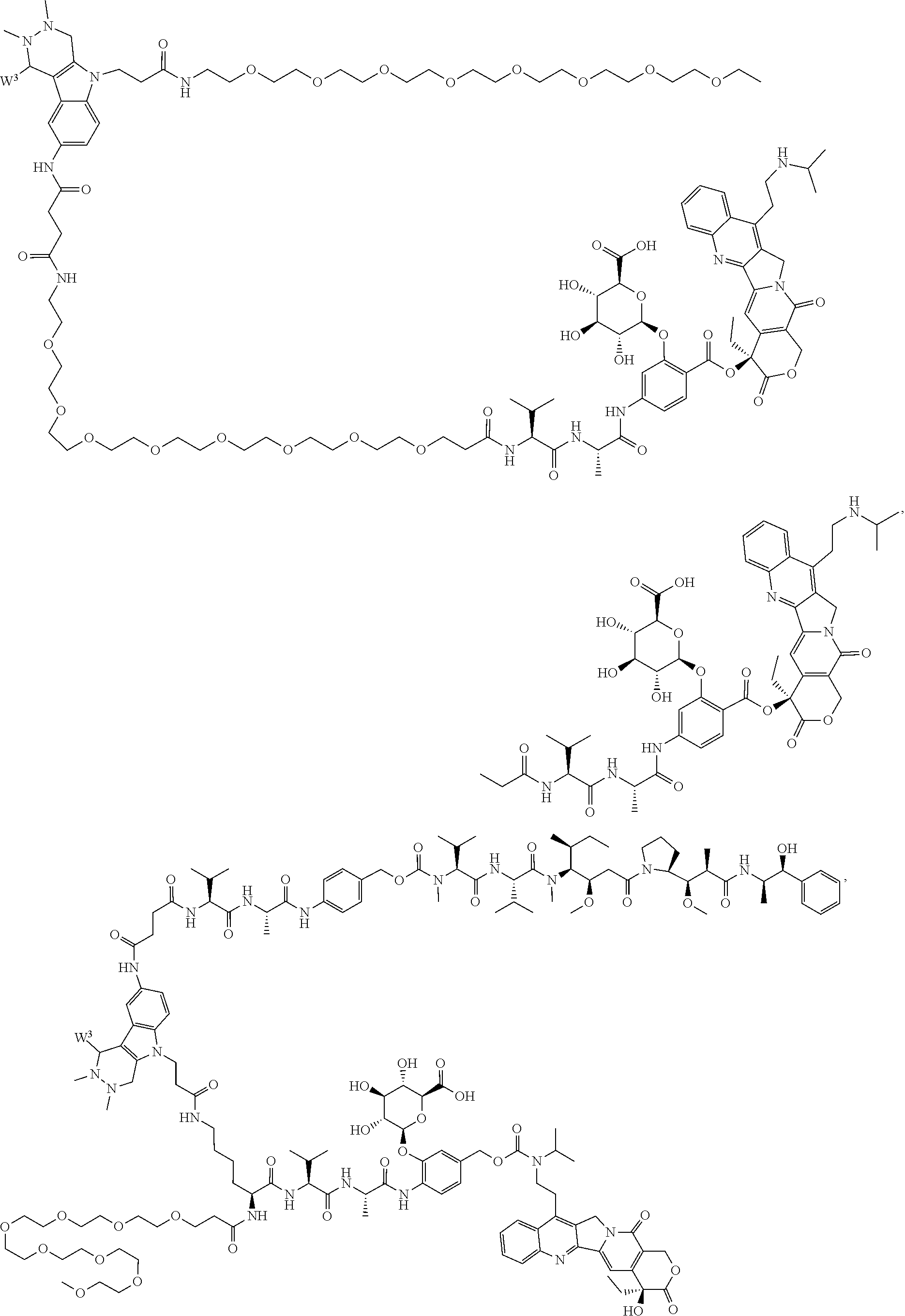

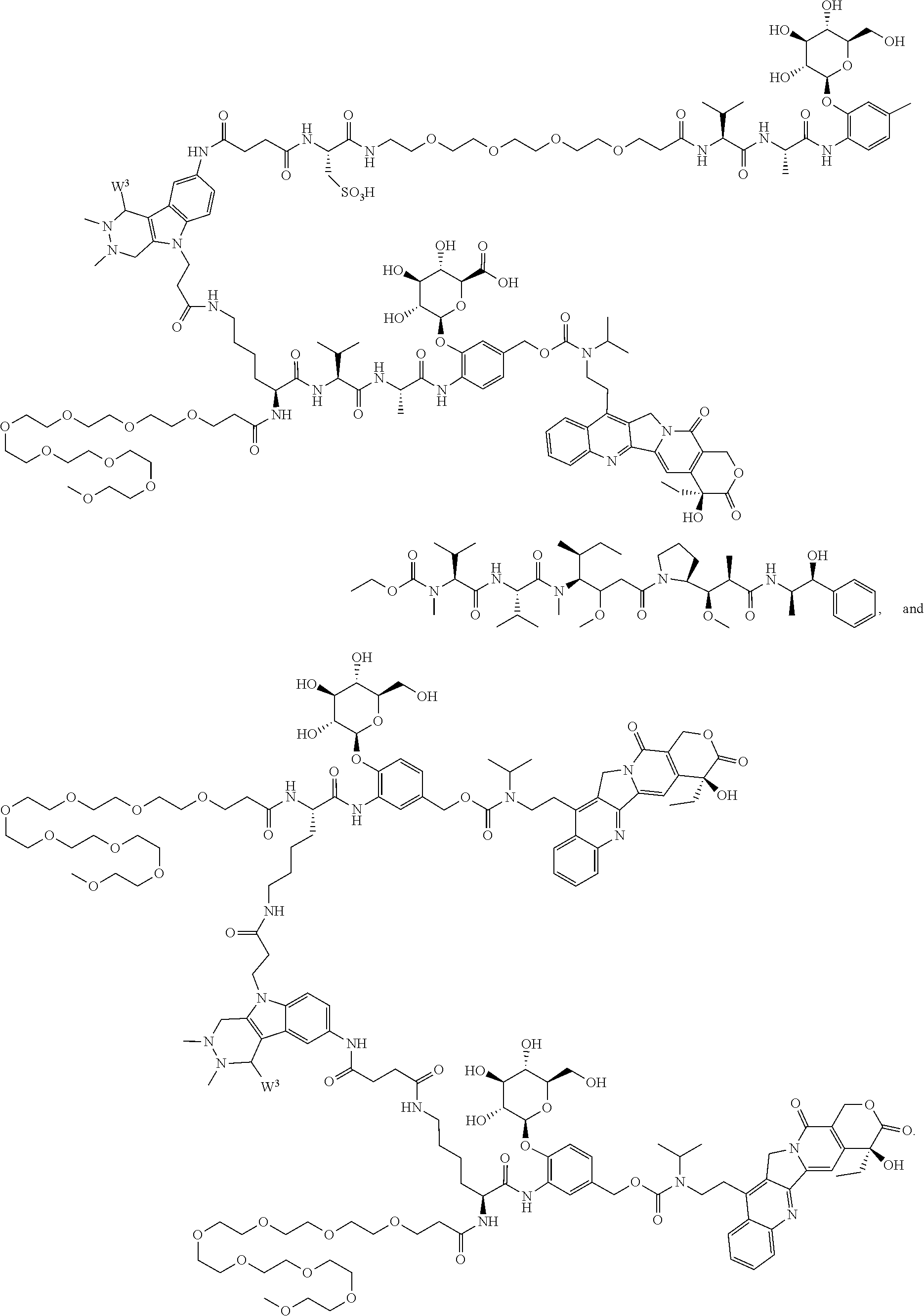

17. The conjugate of any of claims 1-15, wherein the conjugate is selected from:

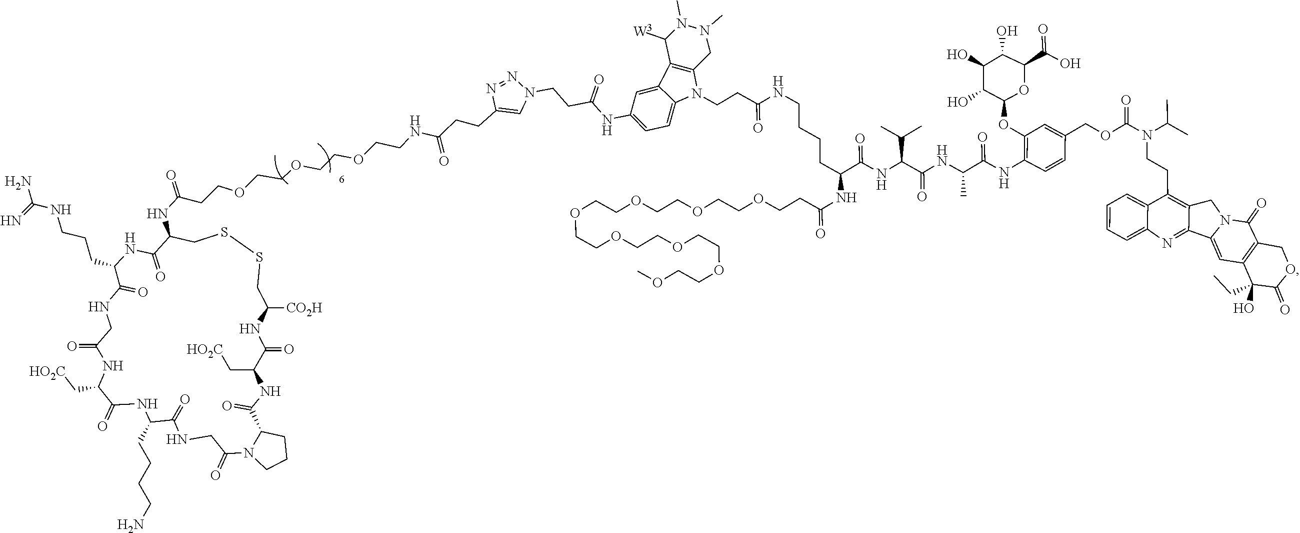

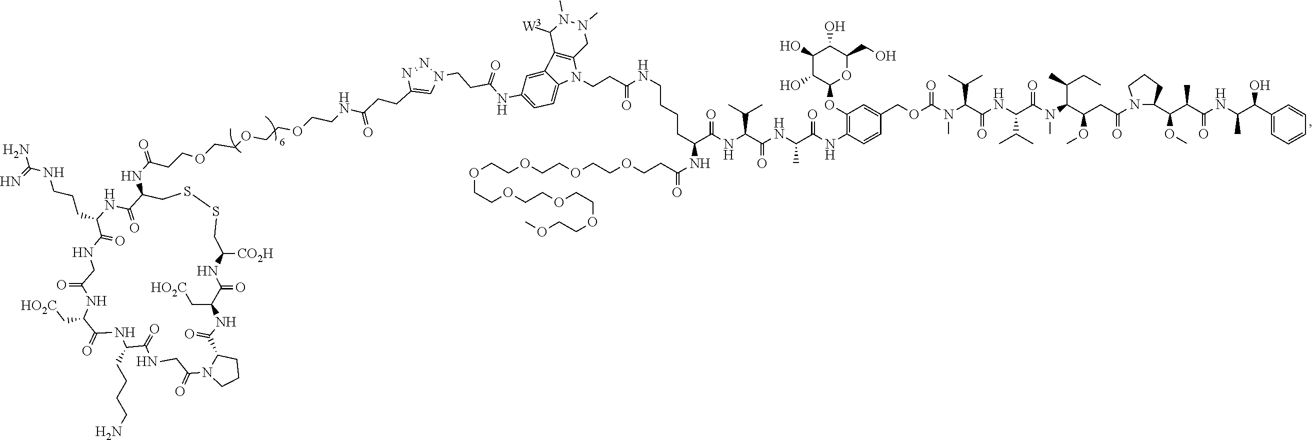

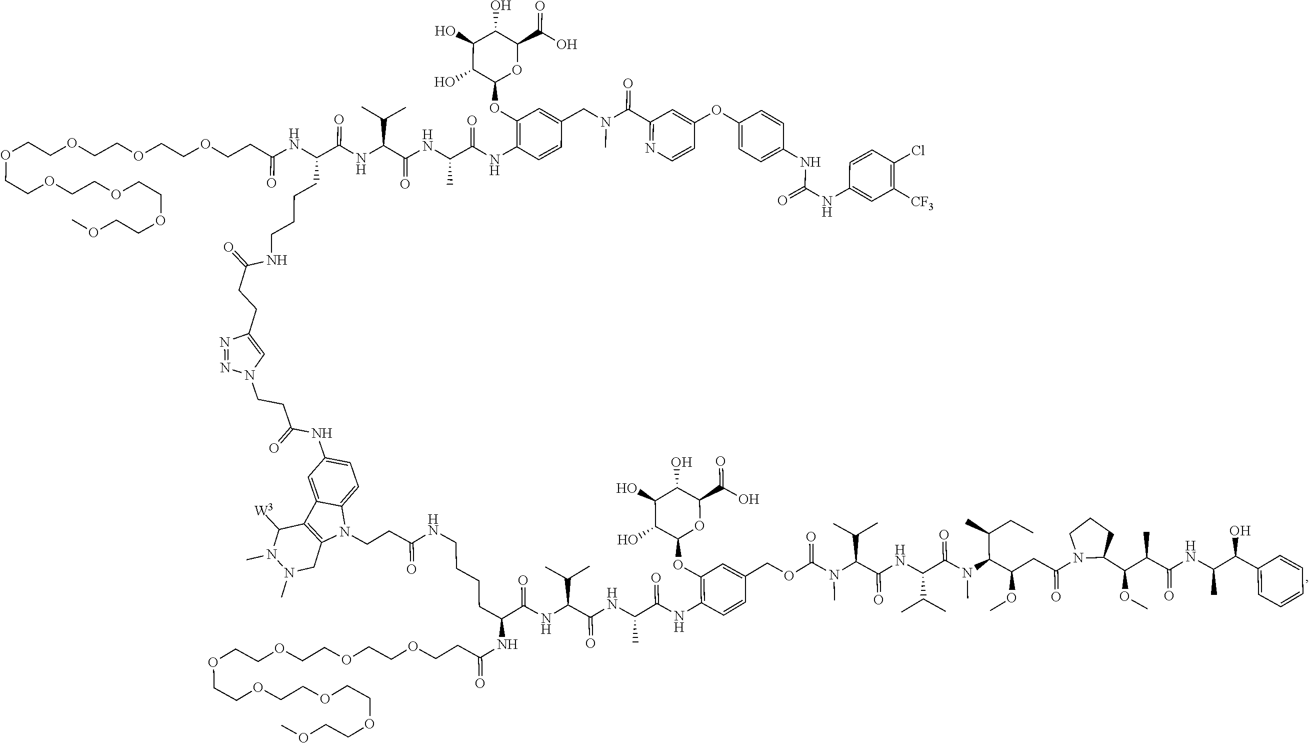

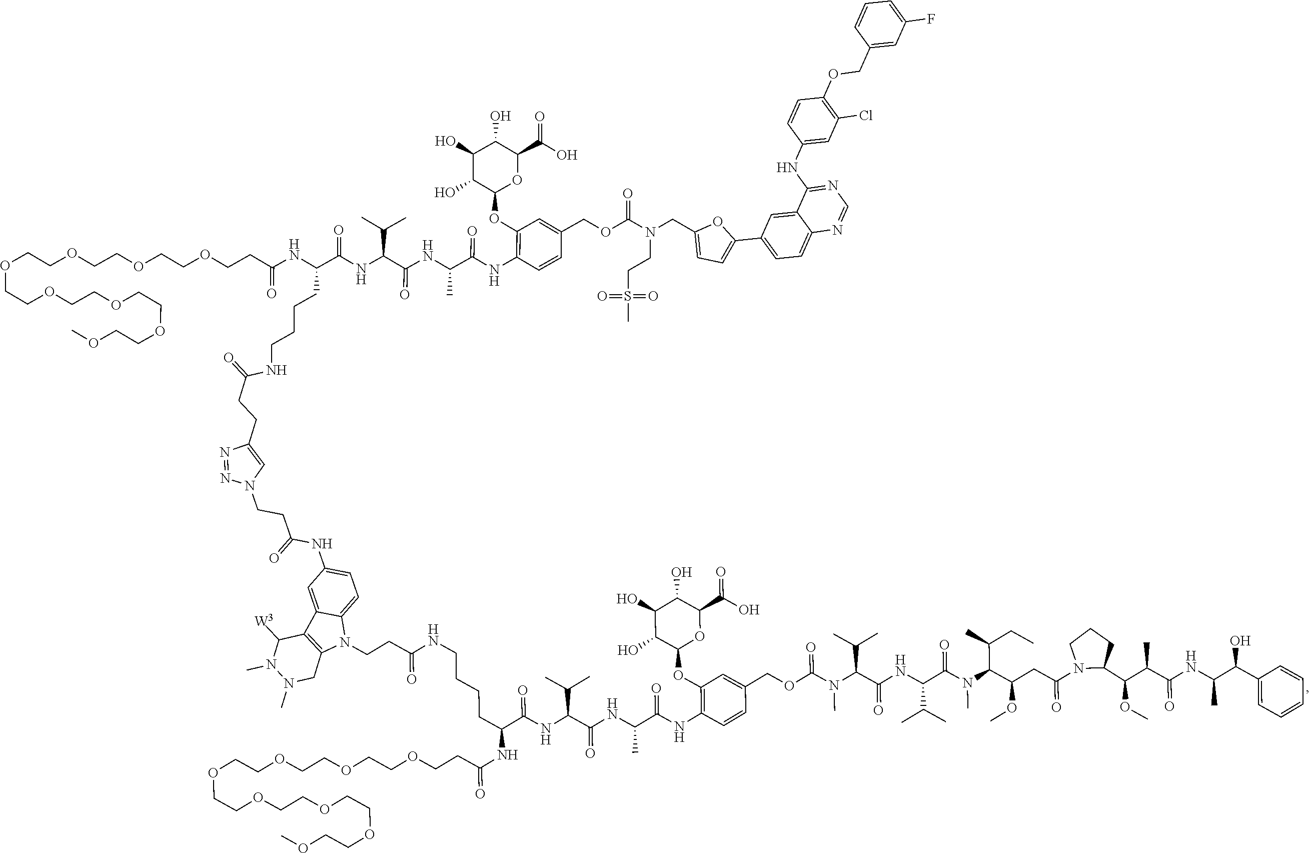

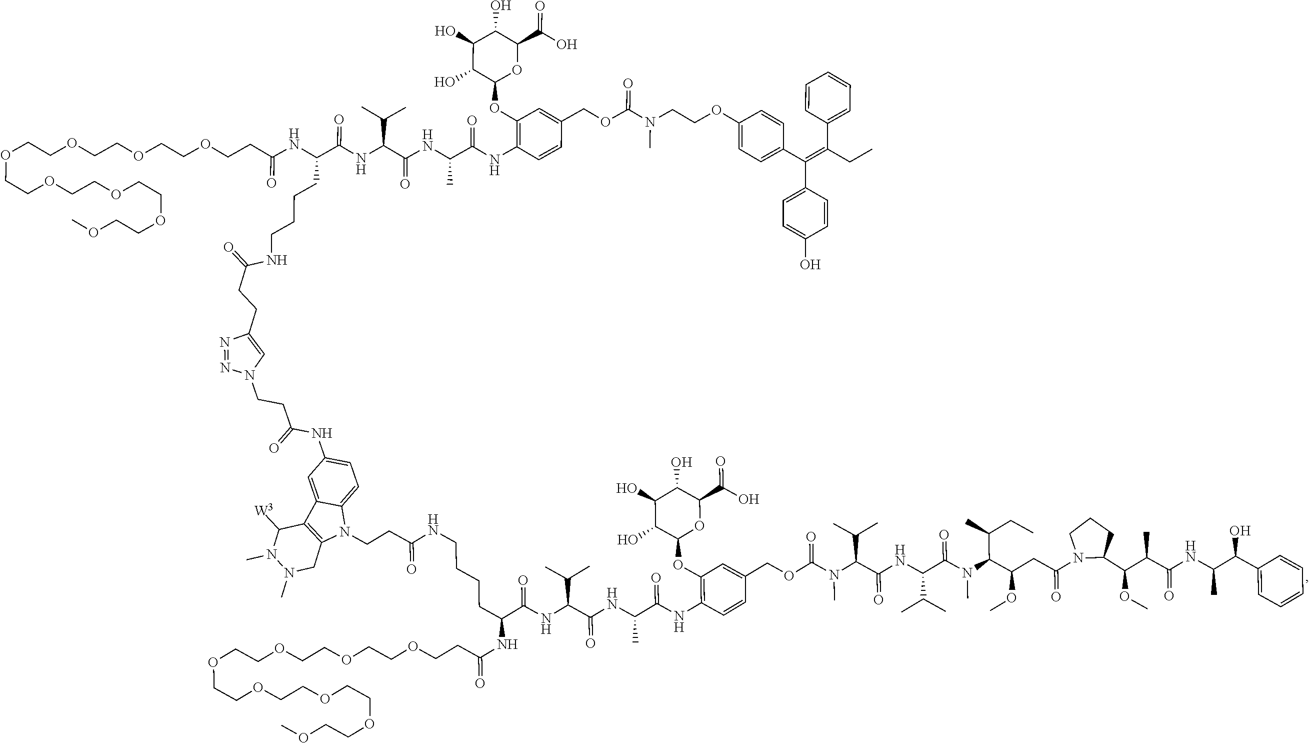

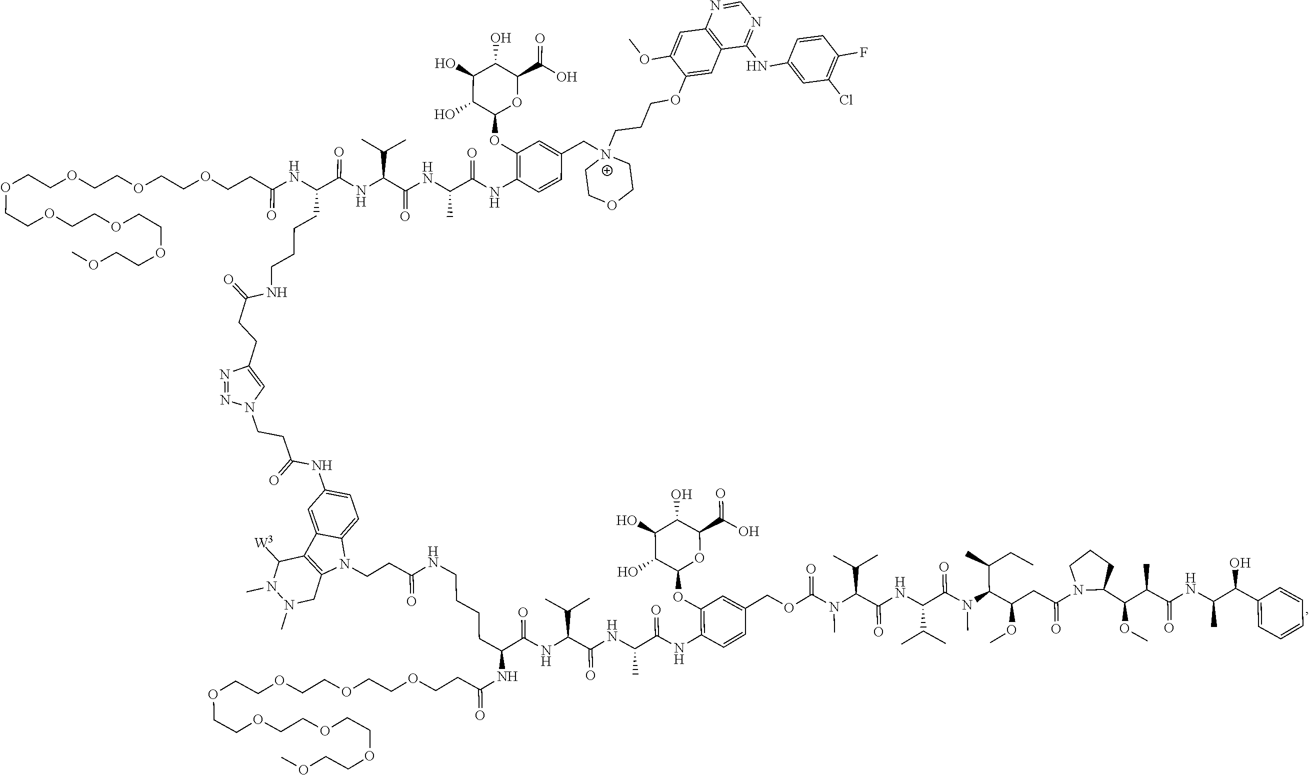

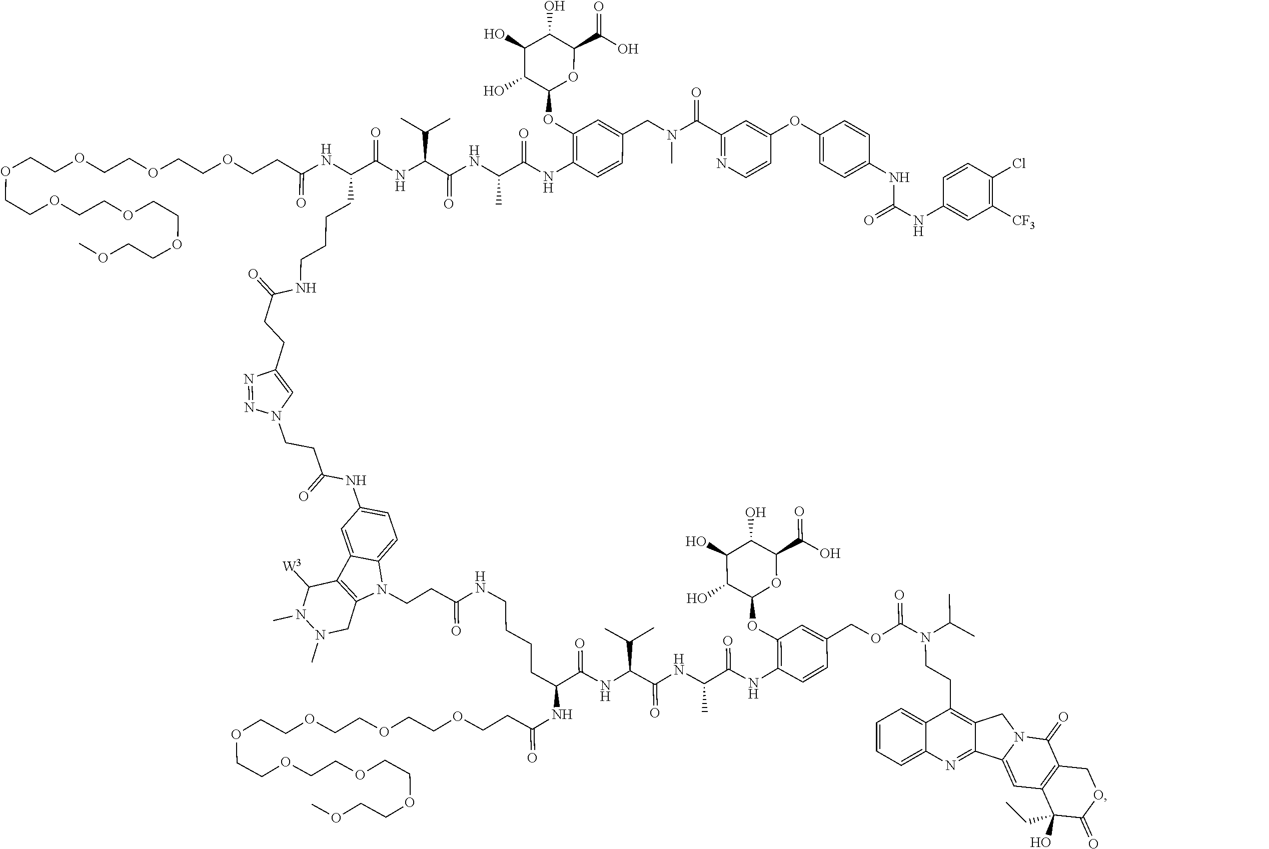

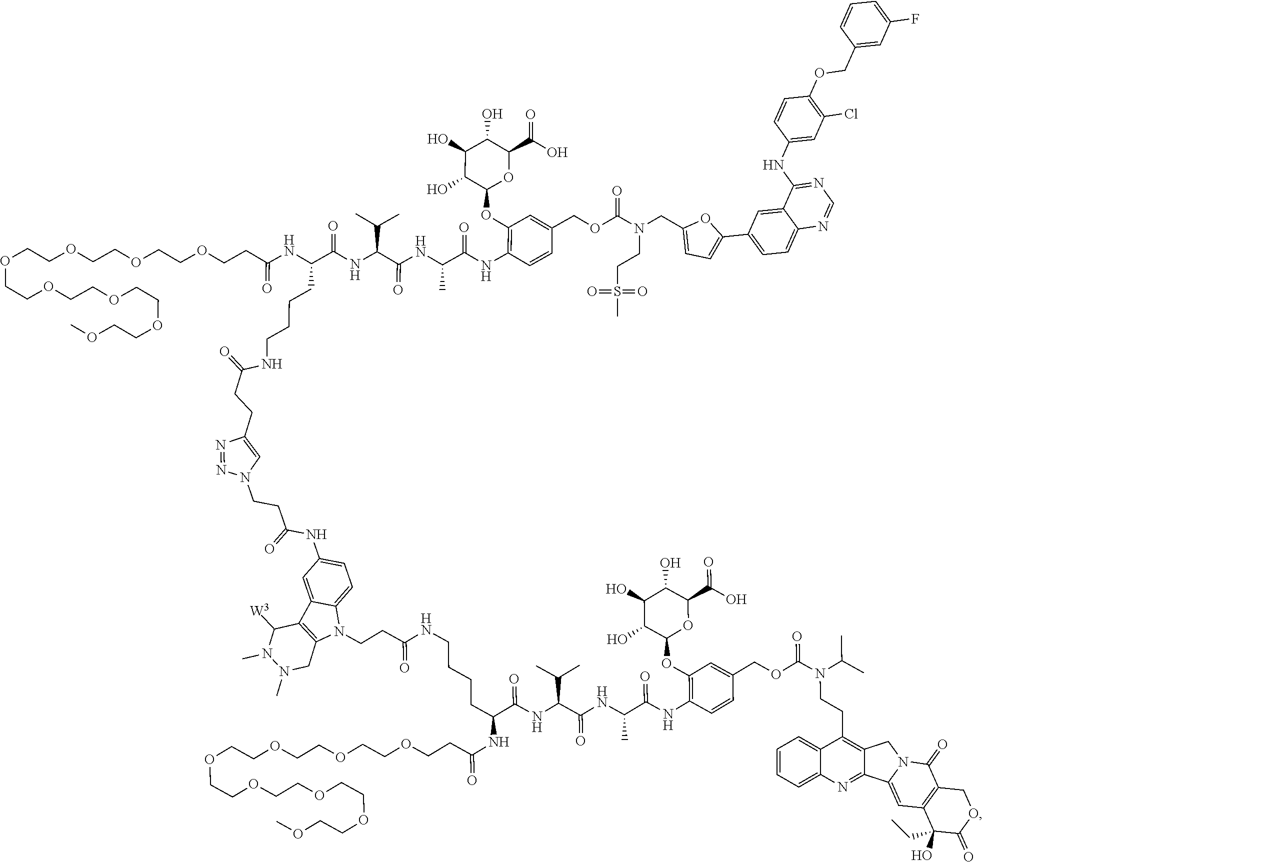

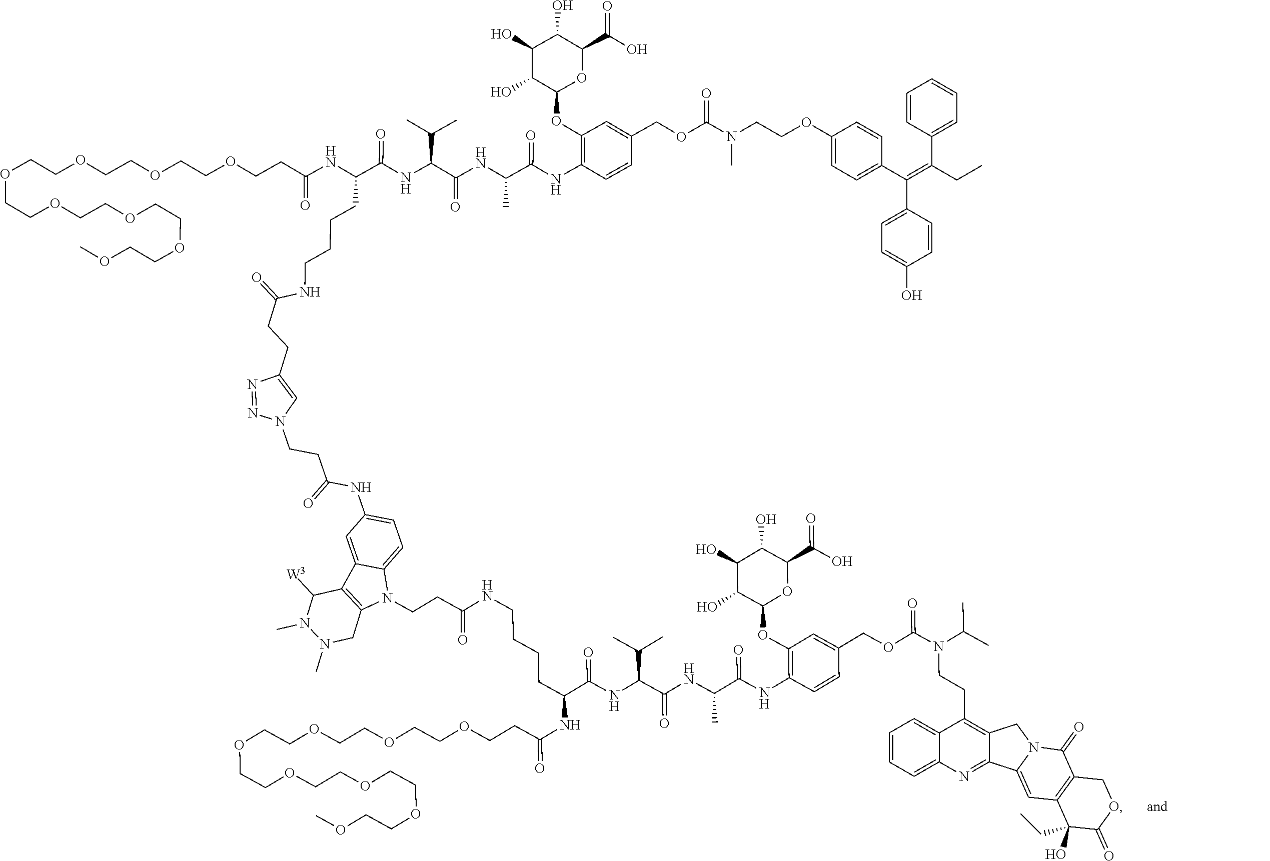

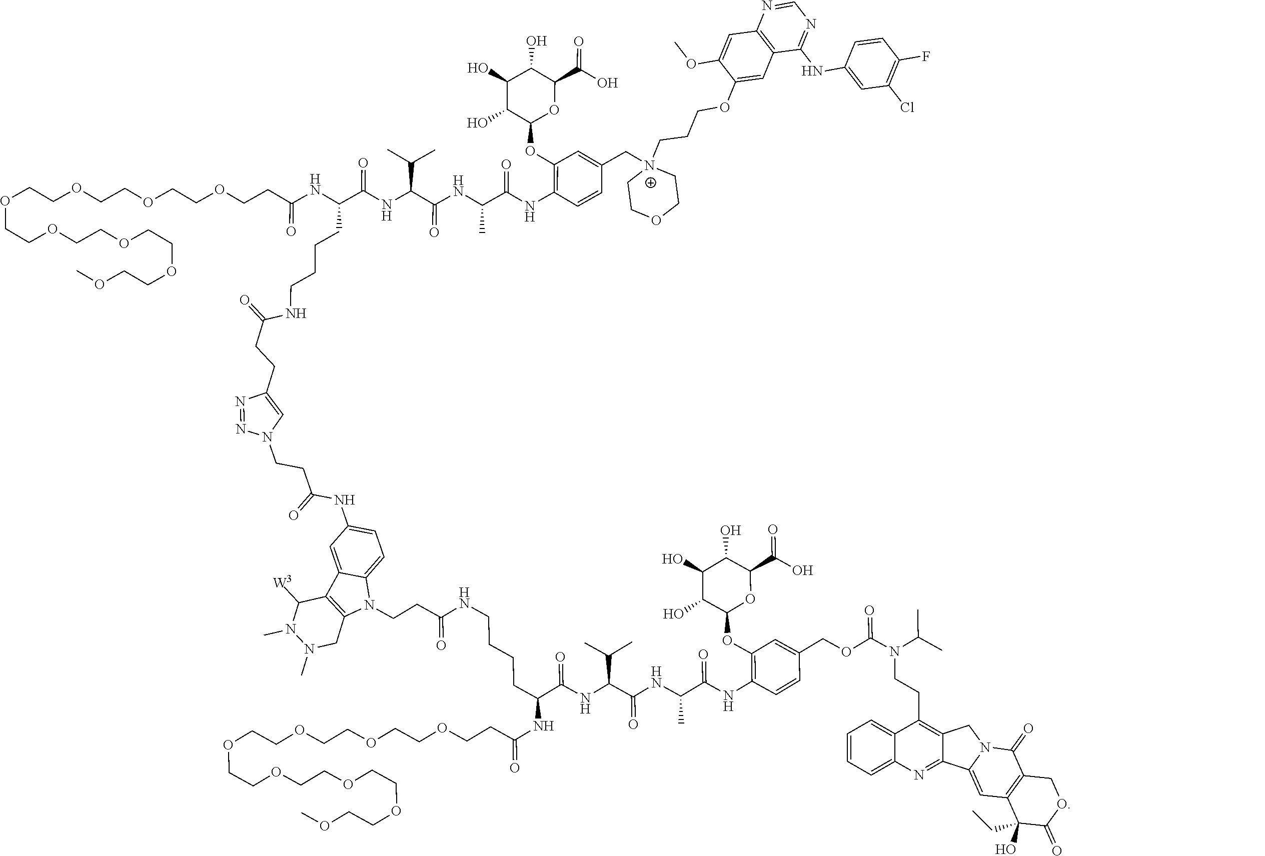

18. A compound of formula (II): 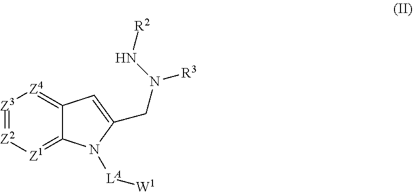
wherein:
Z¹, Z², Z³ and Z⁴ are each independently selected from CR⁴, N and C-L^{B}-W², wherein at least one Z¹, Z², Z³ and Z⁴ is C-L^{B}-W²;
R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L^{A} is a first linker;
L^{B} is a second linker;
W¹ is a first drug; and
W² is a second drug.

19. The compound of claim 18, wherein Z¹ is CR⁴.

20. The compound of claim 18, wherein Z¹ is N.

21. The compound of claim 18, wherein Z³ is C-L^{B}-W².

22. The compound of any of claims 18-21, wherein L^{A} comprises:
-(T¹-V¹)ₐ-(T²-V²)_{b}-(T³-V³)_{c}-(T⁴-V⁴)_{d}-(T⁵-V⁵)ₑ-(T⁶-V⁶)_{f}-,
wherein
a, b, c, d, e and f are each independently 0 or 1;
T¹, T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NRCO—, —C(O)O—, —OC(O)—, —O—, —S—, —S(O)—, —SO₂—, —SO₂NR¹⁵—, —NR″ ⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

23. The compound of claim 22, wherein:
T¹ is selected from a (C₁-C₁₂)alkyl and a substituted (C₁-C₁₂)alkyl;
T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PHP, an acetal group, a hydrazine, and an ester; and
V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂—, and —P(O)OH—;
wherein:
(PEG)ₙ is 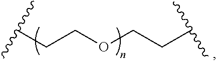
where n is an integer from 1 to 30;
EDA is an ethylene diamine moiety having the following structure: 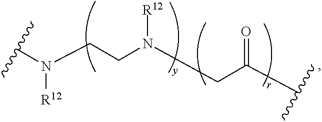
where y is an integer from 1 to 6 and r is 0 or 1;
4-amino-piperidine (4AP) is 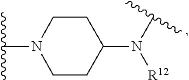
and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring.

24. The compound of any of claims 22-23, wherein T¹, T², T³, T⁴, T⁵ and T⁶ are each optionally substituted with a glycoside.

25. The compound of any of claims 22-23, wherein MABO, MABC, PABO, PABC, PAB, PABA, PAP and PHP are each optionally substituted with a glycoside.

26. The compound of any of claims 24-25, wherein the glycoside is selected from a glucuronide, a galactoside, a glucoside, a mannoside, a fucoside, O-GlcNAc, and O-GalNAc.

27. The compound of any of claims 22-26,
wherein:
T¹ is (C₁-C₁₂)alkyl and V¹ is —CO—;
T² is AA and V² is absent;
T³ is PABC and V³ is absent; and
d, e and f are each 0; or
wherein:
T¹ is (C₁-C₁₂)alkyl and V¹ is —CONH—;
T² is (PEG)ₙ and V² is —CO—;
T³ is AA and V³ is absent;
T⁴ is PABC and V⁴ is absent; and
e and f are each 0; or
wherein:
T¹ is (C₁-C₁₂)alkyl and V¹ is —CO—;
T² is an amino acid analog and V² is —NH—;
T³ is (PEG)ₙ and V³ is —CO—;
T⁴ is AA and V⁴ is absent;
T⁵ is PABC and V⁵ is absent; and
f is 0; or
wherein:
T¹ is (C₁-C₁₂)alkyl and V¹ is —CONH—;
T² is (PEG)ₙ and V² is —CO—;
T³ is AA and V³ is absent;
T⁴ is PABC and V⁴ is absent; and
e and f are each 0; or
wherein:
T¹ is (C₁-C₁₂)alkyl and V¹ is —CONH—;
T² is substituted (C₁-C₁₂)alkyl and V² is —CO—;
T³ is AA and V³ is absent;
T⁴ is PABC and V⁴ is absent; and
e and f are each 0; or
wherein:
T¹ is (C₁-C₁₂)alkyl and V¹ is —CONH—;
T² is (PEG)ₙ and V² is —CO—;
T³ is AA and V³ is absent;
T⁴ is PABA and V⁴ is —CO—;
T⁵ is (C₁-C₁₂)alkyl and V⁵ is absent; and
f is 0; or
wherein:
T¹ is (C₁-C₁₂)alkyl and V¹ is —CO—;
T² is 4AP and V² is —CO—;
T³ is (C₁-C₁₂)alkyl and V³ is —CO—;
T⁴ is AA and V⁴ is absent;
T⁵ is PABC and V⁵ is absent; and
f is 0; or
wherein:
T¹ is (C₁-C₁₂)alkyl and V¹ is —CO—;
T² is 4AP and V² is —CO—;
T³ is (C₁-C₁₂)alkyl and V³ is —O—;
T⁴ is (C₁-C₁₂)alkyl and V⁴ is —CO—;
T⁵ is AA and V⁵ is absent; and
T⁶ is PABC and V⁶ is absent; or
wherein:
T¹ is (C₁-C₁₂)alkyl and V¹ is —CO—;
T² is an amino acid analog and V² is absent;
T³ is AA and V³ is absent;
T⁴ is PABC and V⁴ is absent; and
e and f are each 0; or
wherein:
T¹ is (C₁-C₁₂)alkyl and V¹ is —CONH—;
T² is (PEG)ₙ and V² is —CONH—;
T³ is substituted (C₁-C₁₂)alkyl and V³ is —CO—;
T⁴ is AA and V⁴ is absent;
T⁵ is PABC and V⁵ is absent; and
f is 0; or
wherein:
T¹ is (C₁-C₁₂)alkyl and V¹ is —CO—;
T² is an AA and V² is —NH—;
T³ is (PEG)ₙ and V³ is —CO—;
T⁴ is AA and V⁴ is absent;
T⁵ is PABC and V⁵ is absent; and
f is 0; or
wherein:
T¹ is (C₁-C₁₂)alkyl and V¹ is —CONH—;
T² is (PEG)ₙ and V² is —CO—;
T³ is AA and V³ is absent;
T⁴ is PAP and V⁴ is —C(O)O—; and
e and f are each 0; or
wherein:
T¹ is (C₁-C₁₂)alkyl and V¹ is —CONH—;
T² is substituted (C₁-C₁₂)alkyl and V² is —CO—;
T³ is AA and V³ is absent;
T⁴ is PABC and V⁴ is absent; and
e and f are each 0; or
wherein:
T¹ is (C₁-C₁₂)alkyl and V¹ is —CONH—;
T² is substituted (C₁-C₁₂)alkyl and V² is —CO—;
T³ is PABC and V³ is absent; and
d, e and f are each 0.

28. The compound of any of claims 18-27, wherein L^{B} comprises:
-(T⁷-V⁷)_{g}-(T⁸-V⁸)ₕ-(T⁹-V⁹)ᵢ-(T¹⁰-V¹⁰)ⱼ-(T¹¹-V¹¹)ₖ(T¹²-V¹²)ₗ-(T¹³-V¹³)ₘ,
wherein
g, h, i, j, k and 1 are each independently 0 or 1;
T⁷, T⁸, T⁹, T¹⁰, T¹¹, T¹² and T¹³ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V⁷, V⁸, V⁹, V¹⁰, V¹¹, V¹² and V¹³ are each independently selected from the group consisting of a covalent bond, —CO—, —NR¹⁵—, —NR¹⁵(CH₂)_{q}—, —NR¹⁵(C₆H₄)—, —CONR¹⁵—, —NRCO—, —C(O)O—, —OC(O)—, —O—, —S—, —S(O)—, —SO₂—, —SO₂NR¹⁵—, —NR″ ⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

29. The compound of claim 28, wherein T⁷, T⁸, T⁹, T¹⁰, T¹¹. T¹² and T¹³ are each optionally substituted with a glycoside.

30. The compound of claim 28, wherein MABO, MABC, PABO, PABC, PAB, PABA, PAP and PHP are each optionally substituted with a glycoside.

31. The compound of any of claims 29-30, wherein the glycoside is selected from a glucuronide, a galactoside, a glucoside, a mannoside, a fucoside, O-GlcNAc, and O-GalNAc.

32. The compound of any of claims 28-31,
wherein:
T⁷ is absent and V⁷ is —NHCO—;
T⁸ is (C₁-C₁₂)alkyl and V⁸ is —CO—;
T⁹ is AA and V⁹ is absent;
T¹⁰ is PABC and V¹⁰ is absent; and
k, l and m are each 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is AA and V¹⁰ is absent; and
T¹¹ is PABC and V¹¹ is absent; and
l and m are each 0; or
wherein:
T⁷ is absent and V⁷ is —NHCO—;
T⁸ is (C₁-C₁₂)alkyl and V⁸ is —CO—;
T⁹ is an amino acid analog and V⁹ is —NH—;
T¹⁰ is (PEG)ₙ and V¹⁰ is —CO—;
T¹¹ is AA and V¹¹ is absent;
T¹² is PABC and V¹² is absent; and
m is 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is AA and V¹⁰ is absent;
T¹¹ is PABC and V¹¹ is absent; and
l and m are each 0; or
wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is AA and V¹⁰ is absent;
T¹¹ is PABC and V¹¹ is absent; and
l and m are each 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is AA and V¹⁰ is absent;
T¹¹ is PABA and V¹¹ is —CO—;
T¹² is (C₁-C₁₂)alkyl and V¹² is absent; and
m is 0; or
wherein:
T⁷ is absent and V⁷ is —NHCO—;
T⁸ is (C₁-C₁₂)alkyl and V⁸ is —CO—;
T⁹ is 4AP and V⁹ is —CO—;
T¹⁰ is (C₁-C₁₂)alkyl and V¹⁰ is —CO—;
T¹¹ is AA and V¹¹ is absent;
T¹² is PABC and V¹² is absent; and
m is 0; or
wherein:
T⁷ is absent and V⁷ is —NHCO—;
T⁸ is (C₁-C₁₂)alkyl and V⁸ is —CO—;
T⁹ is 4AP and V⁹ is —CO—;
T¹⁰ is (C₁-C₁₂)alkyl and V¹⁰ is —O—;
T¹¹ is (C₁-C₁₂)alkyl and V¹¹ is —CO—;
T¹² is AA and V¹² is absent; and
T¹³ PABC and V¹³ is absent; or
wherein:
T⁷ is absent and V⁷ is —NHCO—;
T⁸ is (C₁-C₁₂)alkyl and V⁸ is —CO—;
T⁹ is an amino acid analog and V⁹ is absent;
T¹⁰ is AA and V¹⁰ is absent;
T¹¹ is PABC and V¹¹ is absent; and
l and m are each 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CONH—;
T¹⁰ is substituted (C₁-C₁₂)alkyl and V¹⁰ is —CO—;
T¹¹ is AA and V¹¹ is absent;
T¹² is PABC and V¹² is absent; and
m is 0; or
wherein:
T⁷ is absent and V⁷ is —NHCO—;
T⁸ is (C₁-C₁₂)alkyl and V⁸ is —CO—;
T⁹ is AA and V⁹ is —NH—;
T¹⁰ is (PEG)ₙ and V¹⁰ is —CO—;
T¹¹ is AA and V¹¹ is absent;
T¹² is PABC and V¹² is absent; and
m is 0; or
wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is AA and V¹⁰ is absent;
T¹¹ is PAP and V¹¹ is —C(O)O—; and
l and m are each 0; or
wherein:
T⁷ is absent and V⁷ is —NHCO—;
T⁸ is (C₁-C₁₂)alkyl and V⁸ is —CO—;
T⁹ is AA and V⁹ is absent;
T¹⁰ is PABC and V¹⁰ is absent;
T¹¹ is PAP and V¹¹ is —C(O)O—; and
l and m are each 0; or
wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is PABC and V¹⁰ is absent; and
k, l and m are each 0; or
wherein:
T⁷ is absent and V⁷ is —NHCO—;
T⁸ is (C₁-C₁₂)alkyl and V⁸ is absent;
T⁹ is heteroaryl and V⁹ is absent;
T¹⁰ is (C₁-C₁₂)alkyl and V¹⁰ is —CONH—;
T¹¹ is (PEG)ₙ and V¹¹ is —CO—; and
l and m are each 0; or
wherein:
T⁷ is absent and V⁷ is —NHCO—;
T⁸ is (C₁-C₁₂)alkyl and V⁸ is absent;
T⁹ is heteroaryl and V⁹ is absent;
T¹⁰ is (C₁-C₁₂)alkyl and V¹⁰ is —CONH—;
T¹¹ is substituted (C₁-C₁₂)alkyl and V¹¹ is —CO—;
T¹² is AA and V¹² is absent; and
T¹³ PAB and V¹³ is absent; or
wherein:
T⁷ is absent and V⁷ is —NHCO—;
T⁸ is (C₁-C₁₂)alkyl and V⁸ is absent;
T⁹ is heteroaryl and V⁹ is absent;
T¹⁰ is (C₁-C₁₂)alkyl and V¹⁰ is —CONH—;
T¹¹ is substituted (C₁-C₁₂)alkyl and V¹¹ is —CO—;
T¹² is AA and V¹² is absent; and
T¹³ PABC and V¹³ is absent.

33. The compound of any of claims 18-32, wherein the compound is selected from:

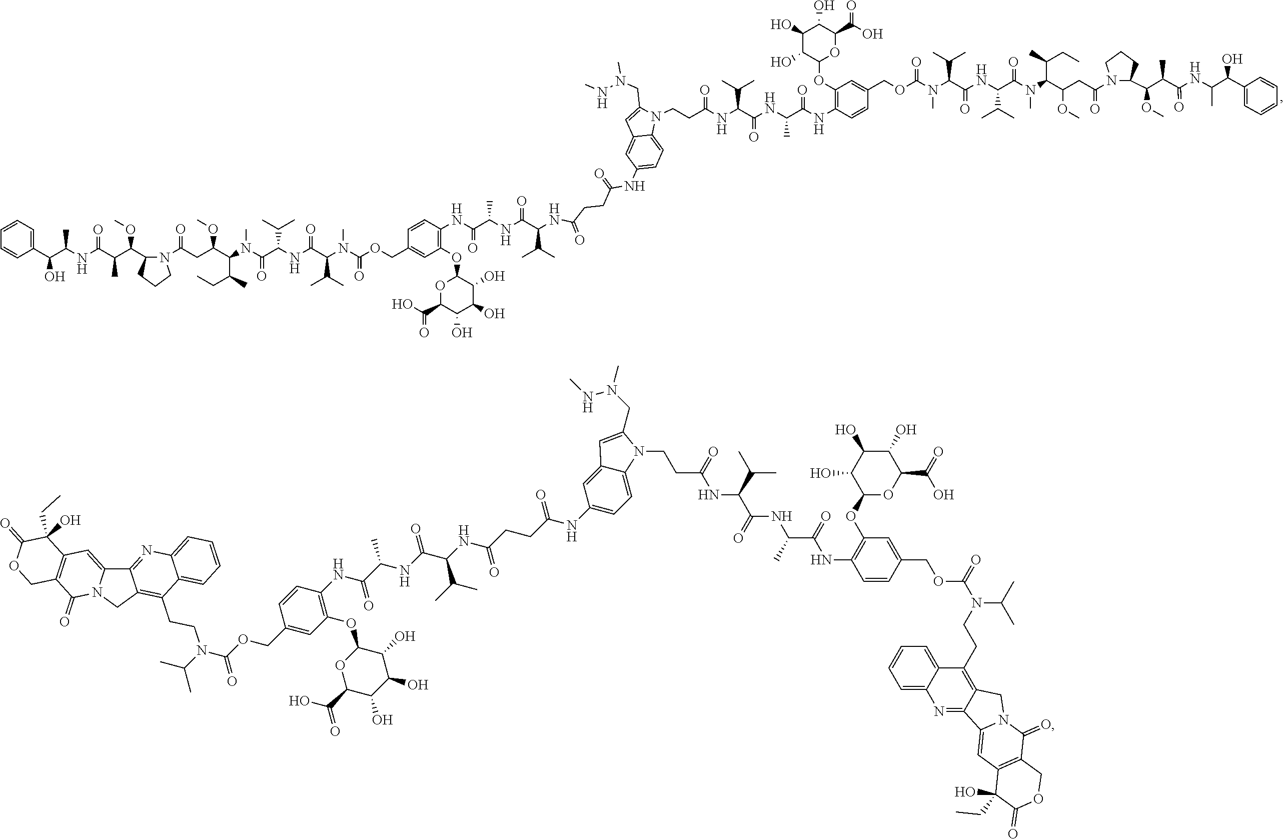

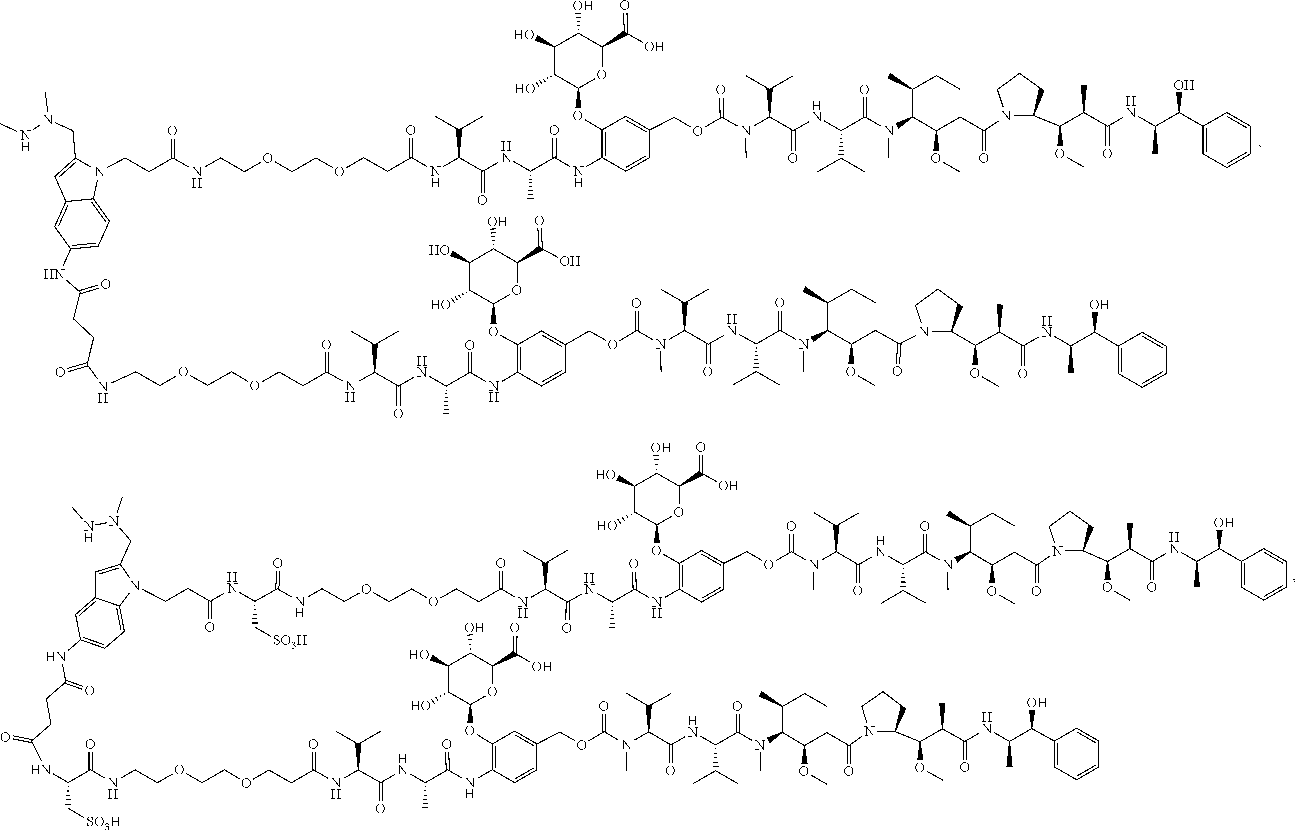

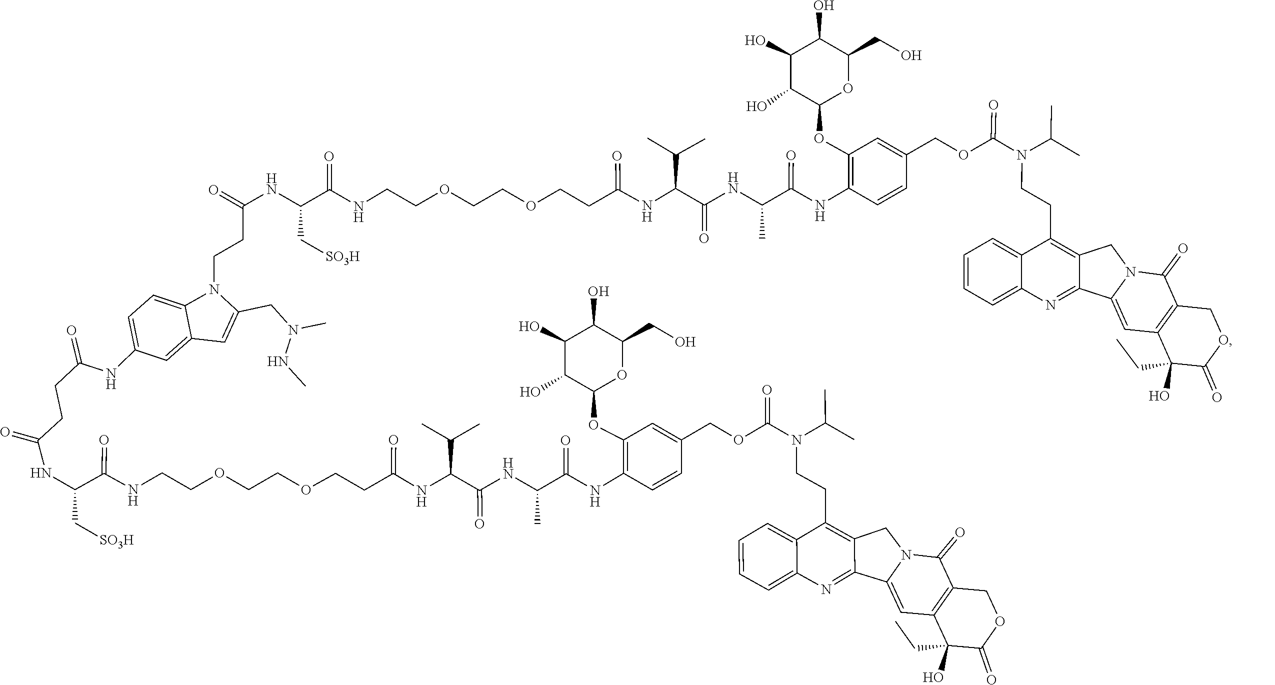 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCOCCOCCNC(=O)[C@H](CS(=O)(=O)O)NC(=O)CCC(=O)Nc2ccc3c(c2)cc(CN(C)NC)n3CCC(=O)N[C@@H](CS(=O)(=O)O)C(=O)NCCOCCOCCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](CO)[C@H](O)[C@H](O)[C@H]2O)C(C)C)C(C)C)c(O[C@@H]2O[C@H](CO)[C@H](O)[C@H](O)[C@H]2O)c1)C(C)C]

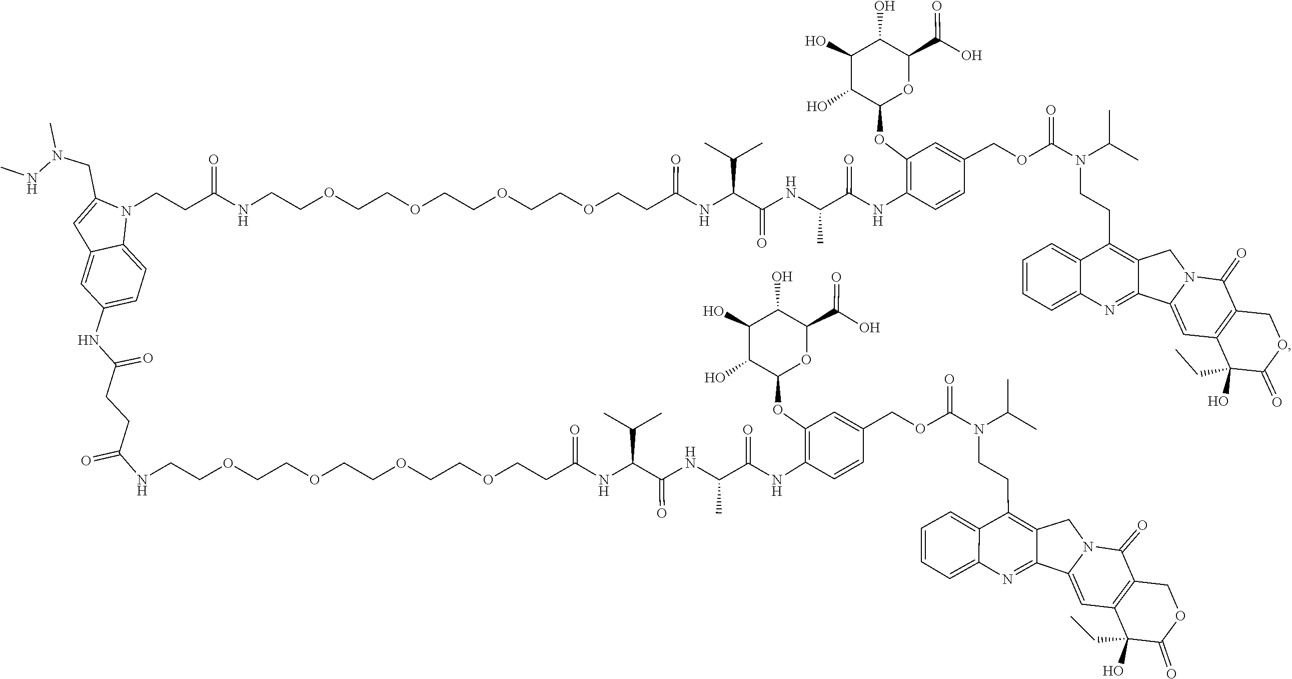 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCOCCOCCOCCOCCNC(=O)CCC(=O)Nc2ccc3c(c2)cc(CN(C)NC)n3CCC(=O)NCCOCCOCCOCCOCCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)c(O[C@@H]2O[C@H](C(=O)O)[C@@H](O)[C@H](O)[C@H]2O)c1)C(C)C]

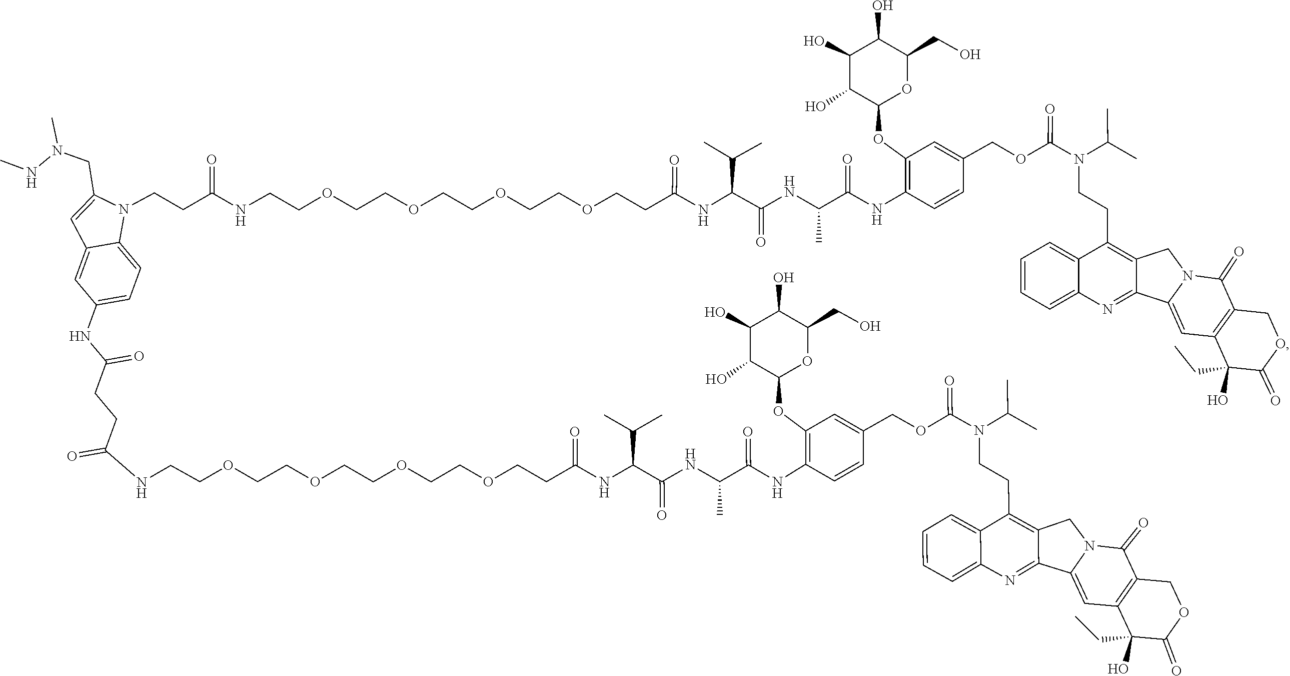 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCOCCOCCOCCOCCNC(=O)CCC(=O)Nc2ccc3c(c2)cc(CN(C)NC)n3CCC(=O)NCCOCCOCCOCCOCCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](CO)[C@H](O)[C@H](O)[C@H]2O)C(C)C)C(C)C)c(O[C@@H]2O[C@H](CO)[C@H](O)[C@H](O)[C@H]2O)c1)C(C)C]

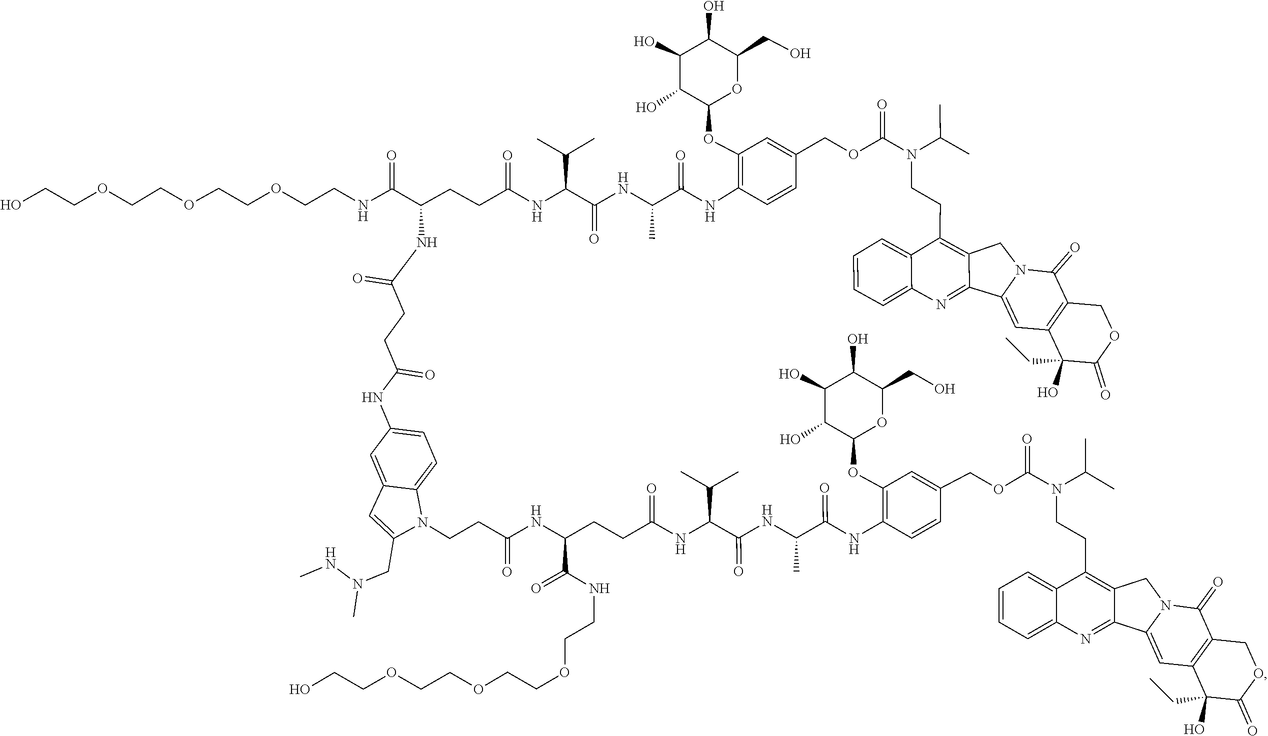 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CC[C@H](NC(=O)CCC(=O)Nc2ccc3c(c2)cc(CN(C)NC)n3CCC(=O)N[C@@H](CCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](CO)[C@H](O)[C@H](O)[C@H]2O)C(C)C)C(=O)NCCOCCOCCOCCO)C(=O)NCCOCCOCCOCCO)C(C)C)c(O[C@@H]2O[C@H](CO)[C@H](O)[C@H](O)[C@H]2O)c1)C(C)C]

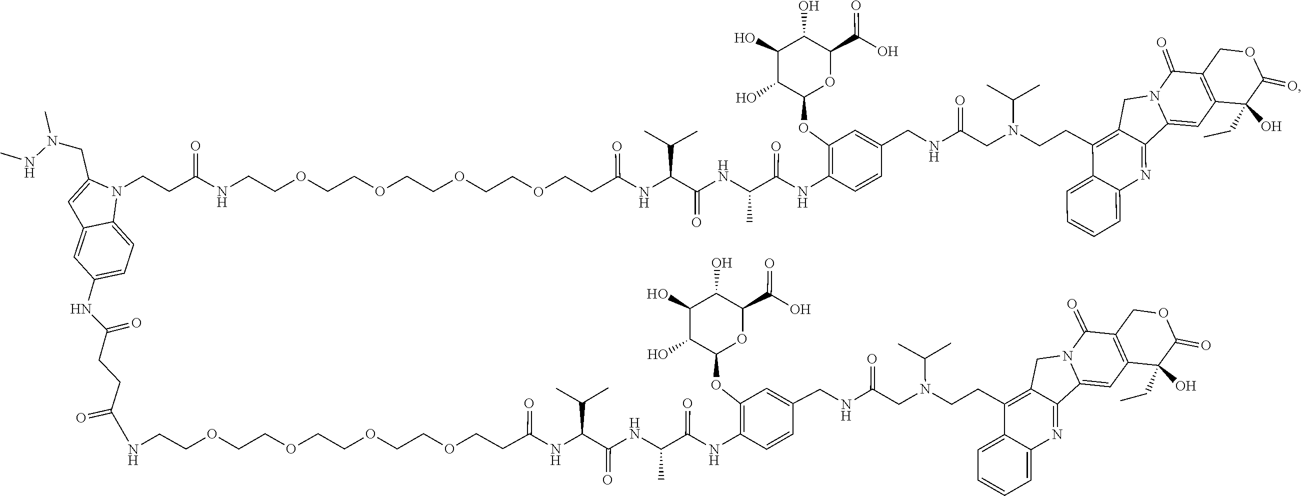 [SMILES: CC[C@@]1(O)C(=O)OCc2c1cc1n(c2=O)Cc2c-1nc1ccccc1c2CCN(CC(=O)NCc1ccc(NC(=O)[C@H](C)NC(=O)[C@@H](NC(=O)CCOCCOCCOCCOCCNC(=O)CCC(=O)Nc2ccc3c(c2)cc(CN(C)NC)n3CCC(=O)NCCOCCOCCOCCOCCC(=O)N[C@H](C(=O)N[C@@H](C)C(=O)Nc2ccc(CNC(=O)CN(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)c(O[C@@H]2O[C@H](C(=O)O)[C@@H](O)[C@H](O)[C@H]2O)c1)C(C)C]

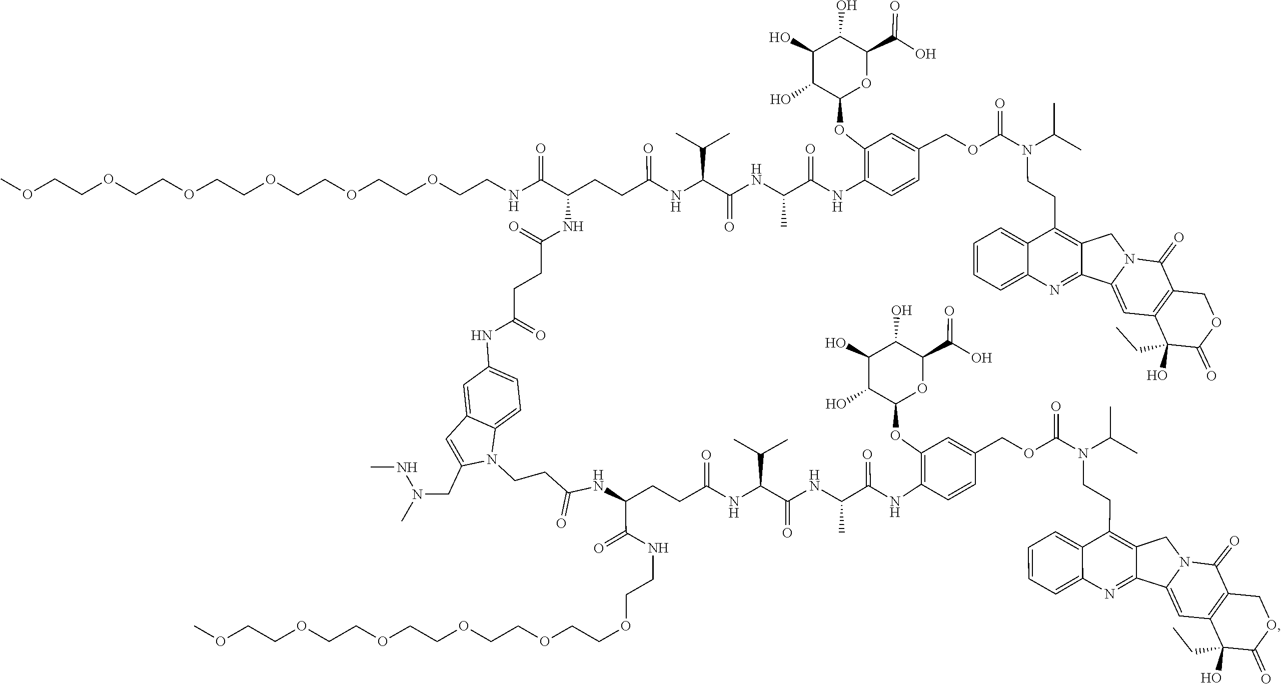 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CC[C@H](NC(=O)CCC(=O)Nc2ccc3c(c2)cc(CN(C)NC)n3CCC(=O)N[C@@H](CCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(=O)NCCOCCOCCOCCOCCOCCOC)C(=O)NCCOCCOCCOCCOCCOCCOC)C(C)C)c(O[C@@H]2O[C@H](C(=O)O)[C@@H](O)[C@H](O)[C@H]2O)c1)C(C)C]

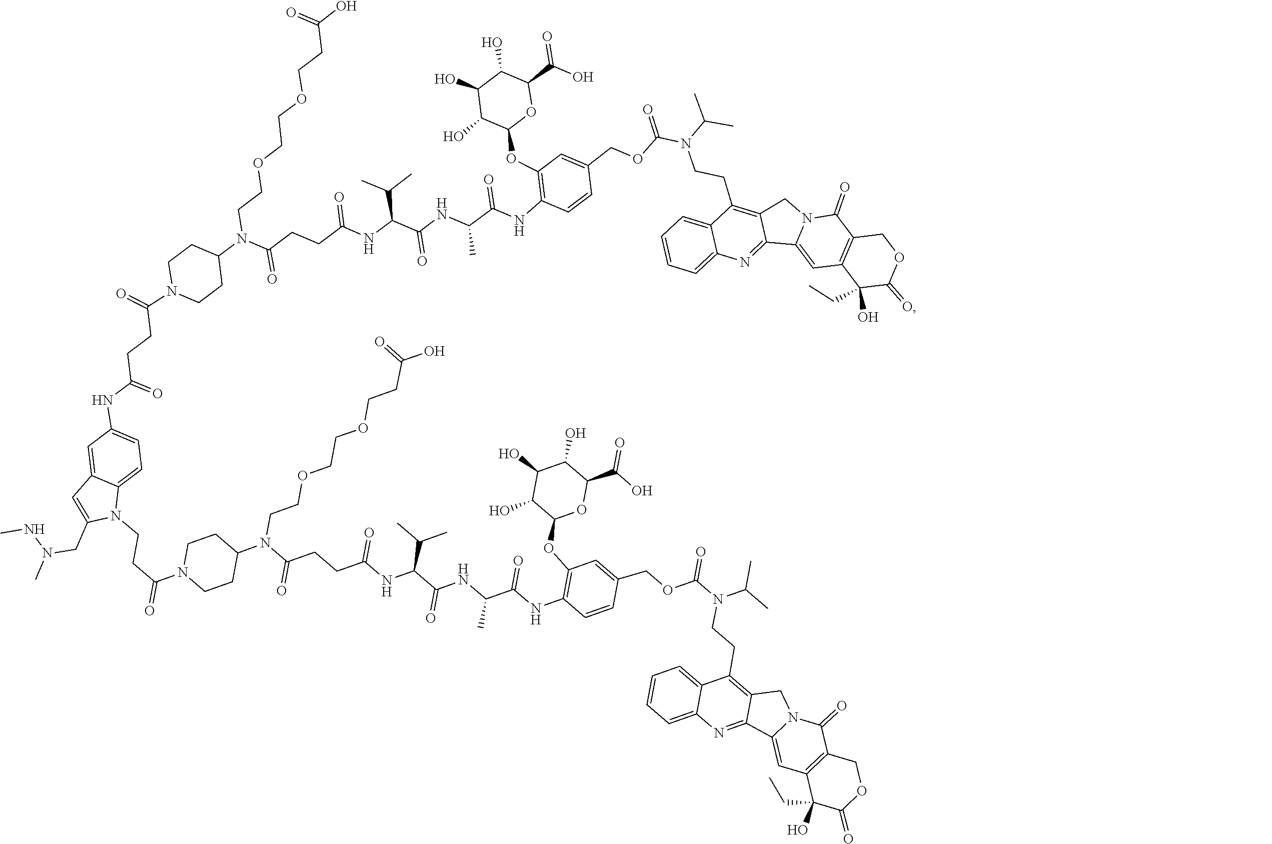 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCC(=O)N(CCOCCOCCC(=O)O)C2CCN(C(=O)CCC(=O)Nc3ccc4c(c3)cc(CN(C)NC)n4CCC(=O)N3CCC(N(CCOCCOCCC(=O)O)C(=O)CCC(=O)N[C@H](C(=O)N[C@@H](C)C(=O)Nc4ccc(COC(=O)N(CCc5c6c(nc7ccccc57)-c5cc7c(c(=O)n5C6)COC(=O)[C@]7(O)CC)C(C)C)cc4O[C@@H]4O[C@H](C(=O)O)[C@@H](O)[C@H](O)[C@H]4O)C(C)C)CC3)CC2)C(C)C)c(O[C@@H]2O[C@H](C(=O)O)[C@@H](O)[C@H](O)[C@H]2O)c1)C(C)C]

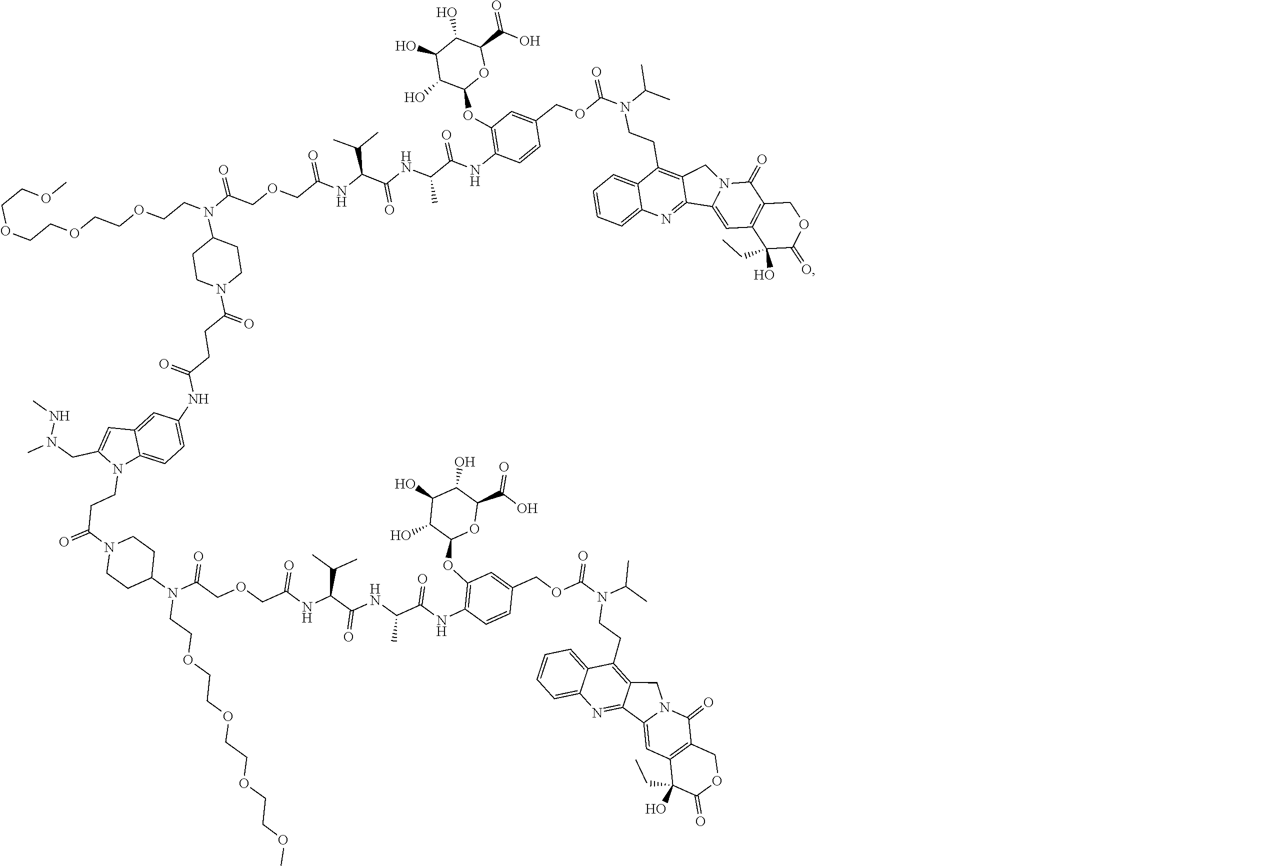 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)COCC(=O)N(CCOCCOCCOCCOC)C2CCN(C(=O)CCC(=O)Nc3ccc4c(c3)cc(CN(C)NC)n4CCC(=O)N3CCC(N(CCOCCOCCOCCOC)C(=O)COCC(=O)N[C@H](C(=O)N[C@@H](C)C(=O)Nc4ccc(COC(=O)N(CCc5c6c(nc7ccccc57)-c5cc7c(c(=O)n5C6)COC(=O)[C@]7(O)CC)C(C)C)cc4O[C@@H]4O[C@H](C(=O)O)[C@@H](O)[C@H](O)[C@H]4O)C(C)C)CC3)CC2)C(C)C)c(O[C@@H]2O[C@H](C(=O)O)[C@@H](O)[C@H](O)[C@H]2O)c1)C(C)C]

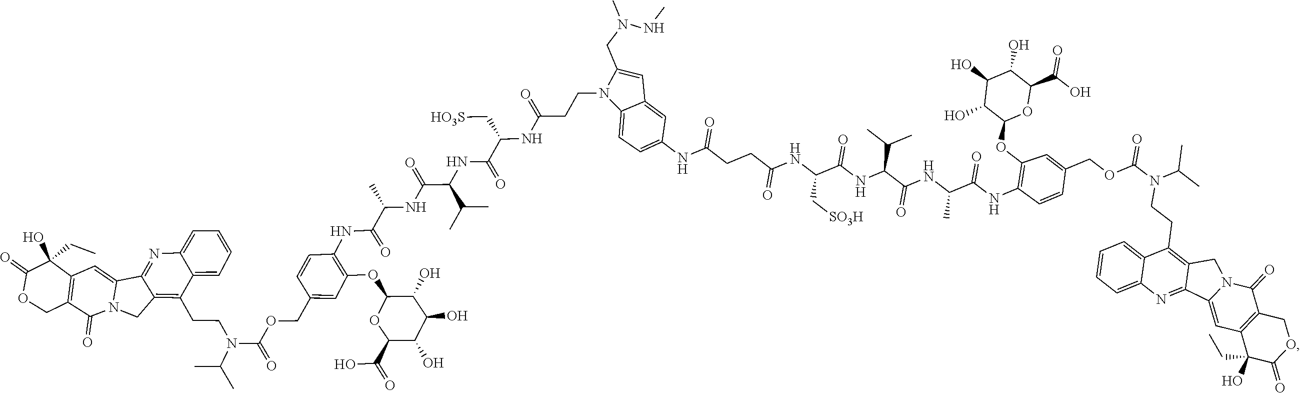 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CS(=O)(=O)O)NC(=O)CCC(=O)Nc2ccc3c(c2)cc(CN(C)NC)n3CCC(=O)N[C@@H](CS(=O)(=O)O)C(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)c(O[C@@H]2O[C@H](C(=O)O)[C@@H](O)[C@H](O)[C@H]2O)c1)C(C)C]

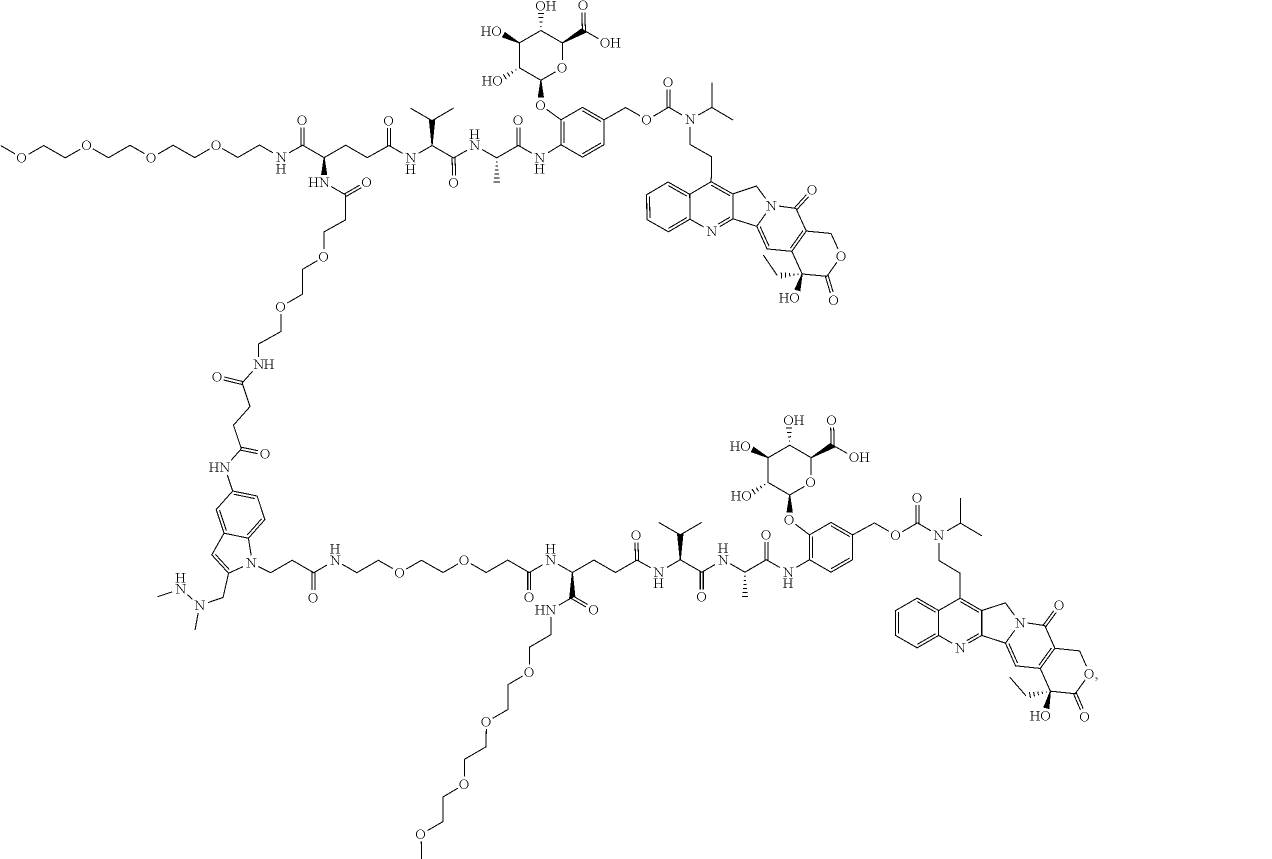 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CC[C@H](NC(=O)CCOCCOCCNC(=O)CCn2c(CN(C)NC)cc3cc(NC(=O)CCC(=O)NCCOCCOCCC(=O)N[C@H](CCC(=O)N[C@H](C(=O)N[C@@H](C)C(=O)Nc4ccc(COC(=O)N(CCc5c6c(nc7ccccc57)-c5cc7c(c(=O)n5C6)COC(=O)[C@]7(O)CC)C(C)C)cc4O[C@@H]4O[C@H](C(=O)O)[C@@H](O)[C@H](O)[C@H]4O)C(C)C)C(=O)NCCOCCOCCOCCOC)ccc32)C(=O)NCCOCCOCCOCCOC)C(C)C)c(O[C@@H]2O[C@H](C(=O)O)[C@@H](O)[C@H](O)[C@H]2O)c1)C(C)C]

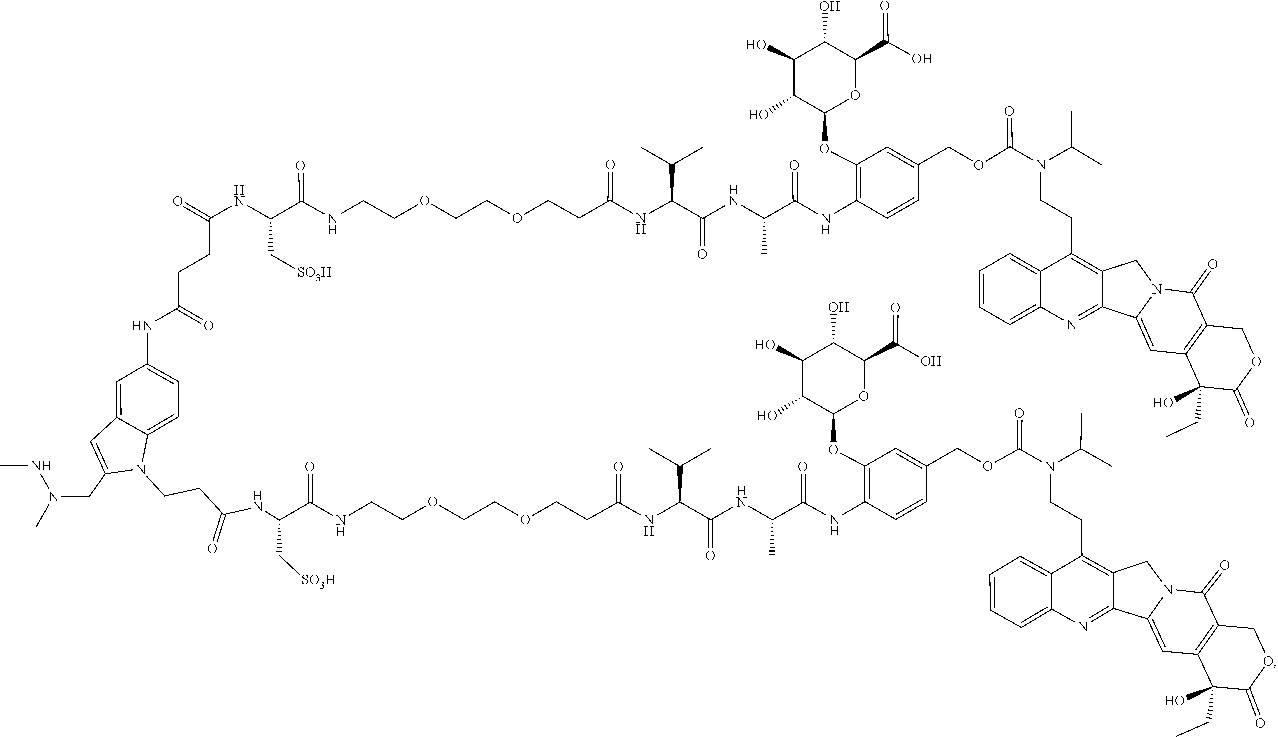 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCOCCOCCNC(=O)[C@H](CS(=O)(=O)O)NC(=O)CCC(=O)Nc2ccc3c(c2)cc(CN(C)NC)n3CCC(=O)N[C@@H](CS(=O)(=O)O)C(=O)NCCOCCOCCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)c(O[C@@H]2O[C@H](C(=O)O)[C@@H](O)[C@H](O)[C@H]2O)c1)C(C)C]

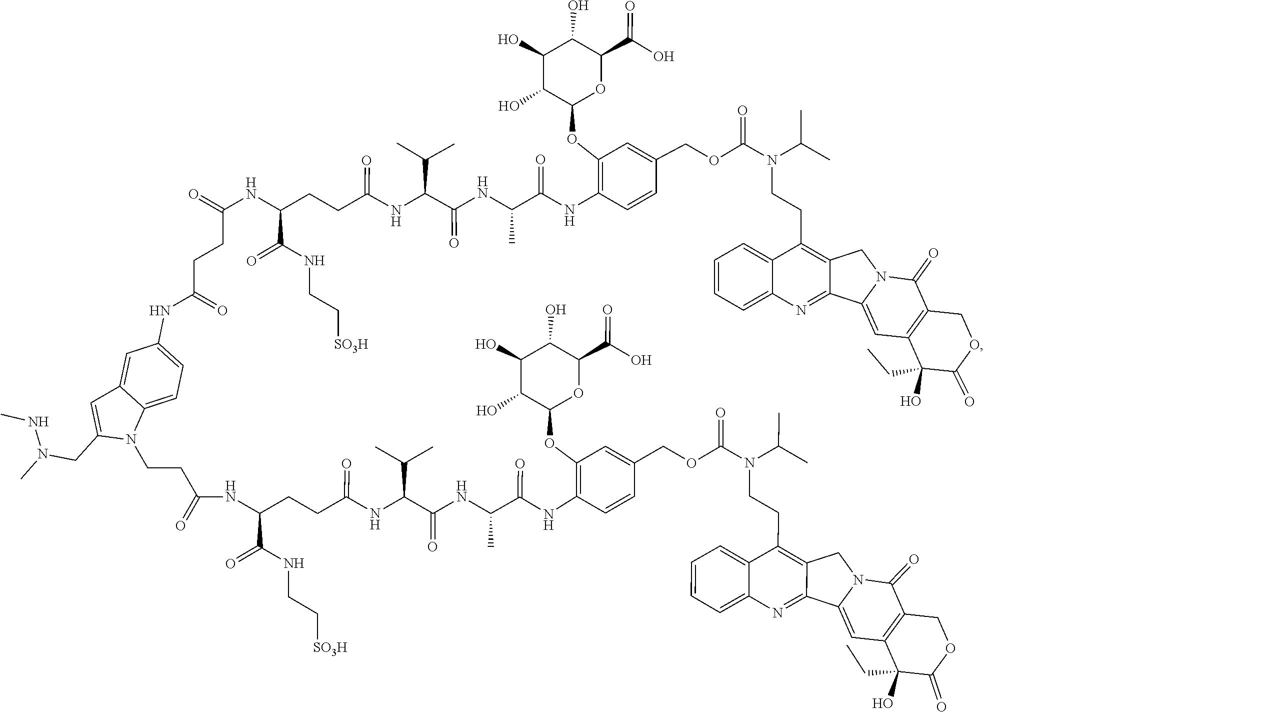 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CC[C@H](NC(=O)CCC(=O)Nc2ccc3c(c2)cc(CN(C)NC)n3CCC(=O)N[C@@H](CCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(=O)NCCS(=O)(=O)O)C(=O)NCCS(=O)(=O)O)C(C)C)c(O[C@@H]2O[C@H](C(=O)O)[C@@H](O)[C@H](O)[C@H]2O)c1)C(C)C]

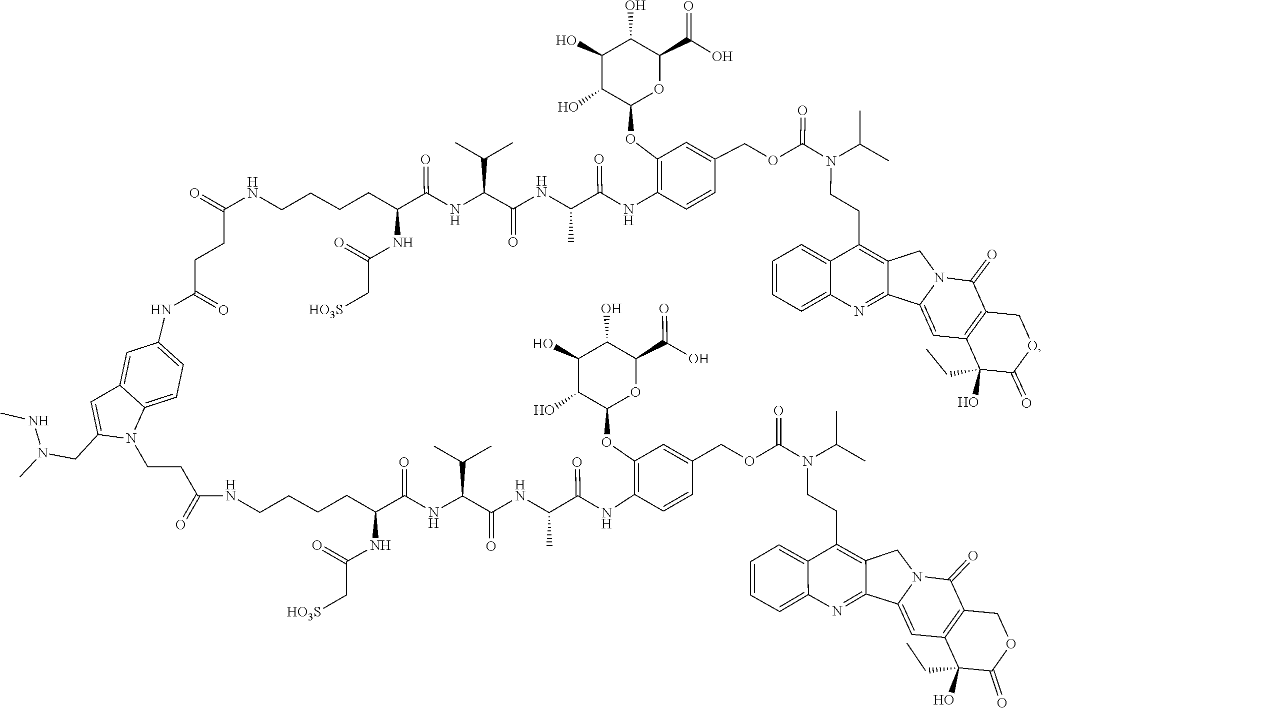 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CCCCNC(=O)CCC(=O)Nc2ccc3c(c2)cc(CN(C)NC)n3CCC(=O)NCCCC[C@H](NC(=O)CS(=O)(=O)O)C(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)NC(=O)CS(=O)(=O)O)C(C)C)c(O[C@@H]2O[C@H](C(=O)O)[C@@H](O)[C@H](O)[C@H]2O)c1)C(C)C]

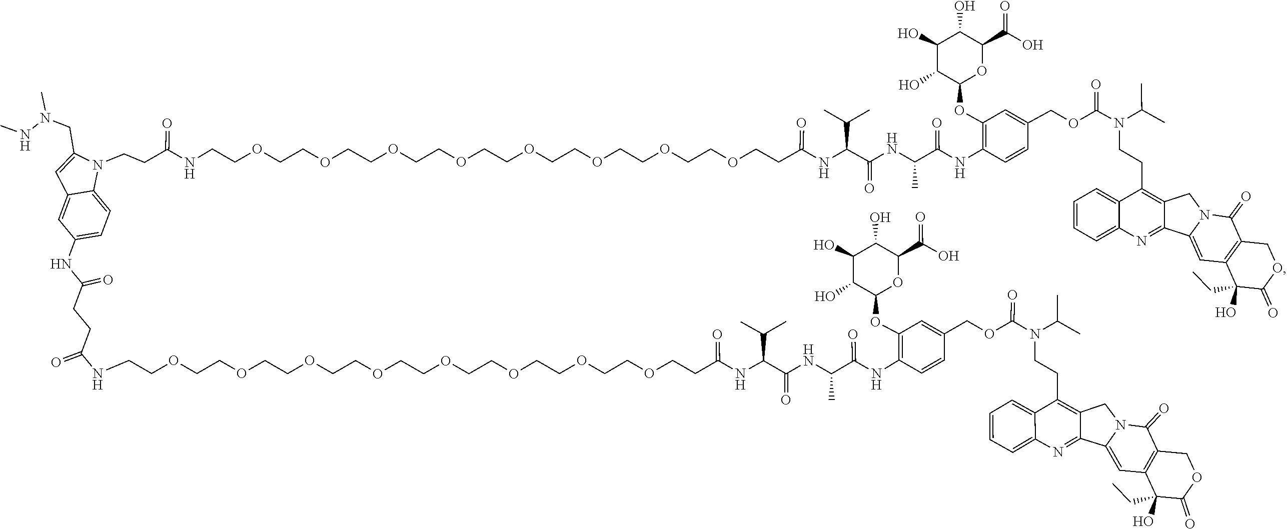 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCOCCOCCOCCOCCOCCOCCOCCOCCNC(=O)CCC(=O)Nc2ccc3c(c2)cc(CN(C)NC)n3CCC(=O)NCCOCCOCCOCCOCCOCCOCCOCCOCCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)c(O[C@@H]2O[C@H](C(=O)O)[C@@H](O)[C@H](O)[C@H]2O)c1)C(C)C]

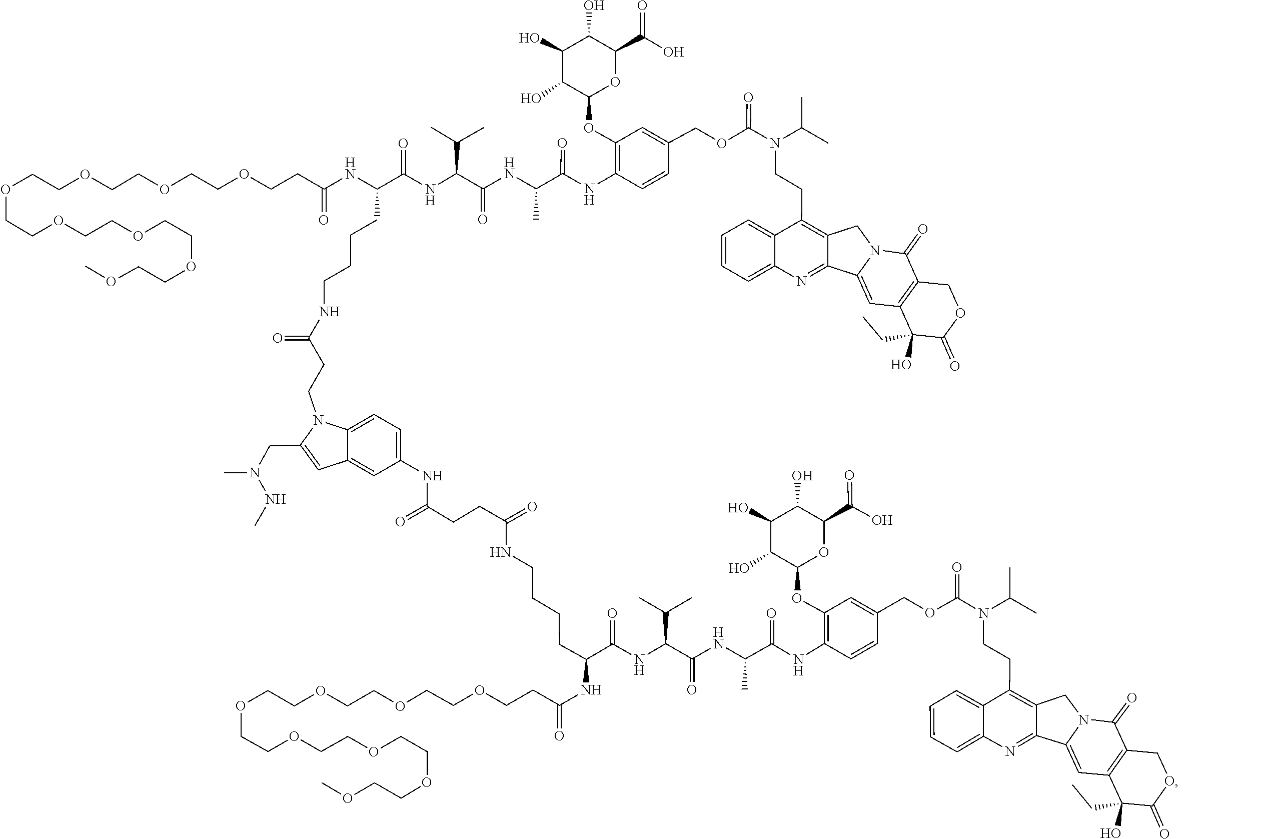 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CCCCNC(=O)CCC(=O)Nc2ccc3c(c2)cc(CN(C)NC)n3CCC(=O)NCCCC[C@H](NC(=O)CCOCCOCCOCCOCCOCCOCCOCCOC)C(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)NC(=O)CCOCCOCCOCCOCCOCCOCCOCCOC)C(C)C)c(O[C@@H]2O[C@H](C(=O)O)[C@@H](O)[C@H](O)[C@H]2O)c1)C(C)C]

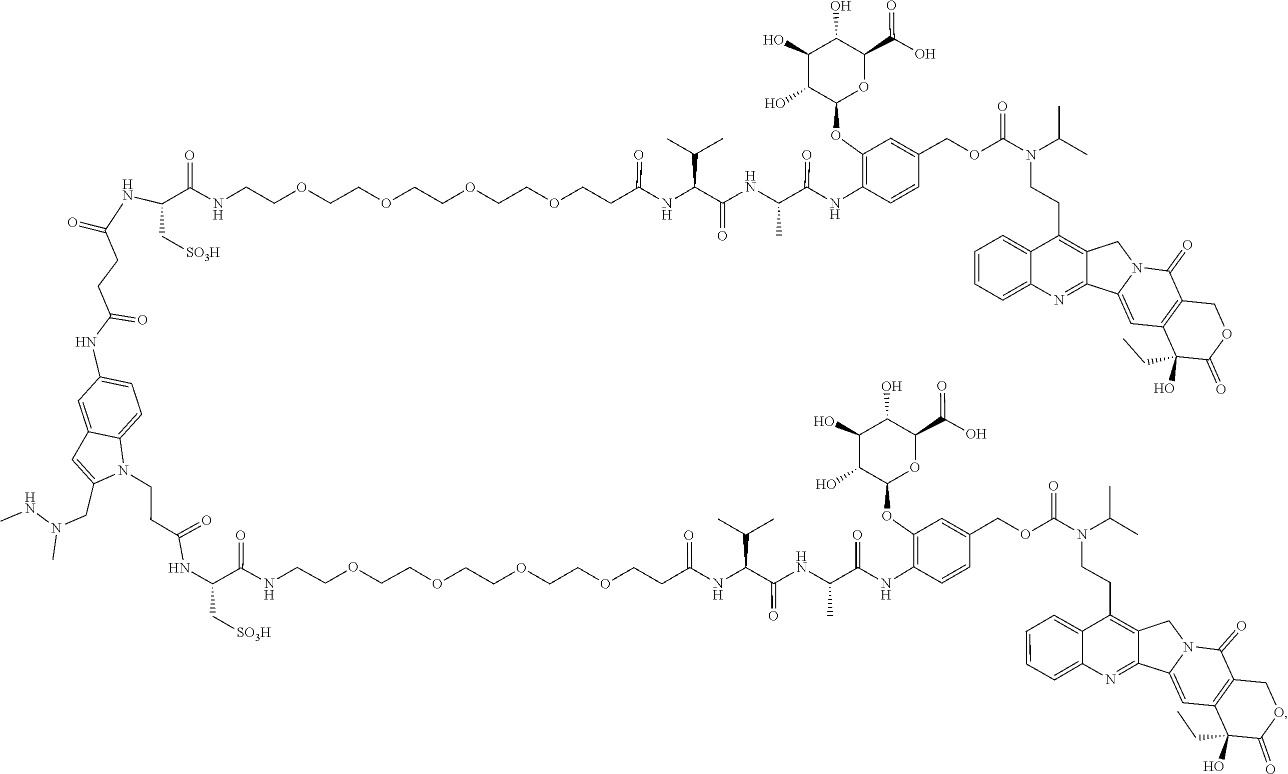 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCOCCOCCOCCOCCNC(=O)[C@H](CS(=O)(=O)O)NC(=O)CCC(=O)Nc2ccc3c(c2)cc(CN(C)NC)n3CCC(=O)N[C@@H](CS(=O)(=O)O)C(=O)NCCOCCOCCOCCOCCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)c(O[C@@H]2O[C@H](C(=O)O)[C@@H](O)[C@H](O)[C@H]2O)c1)C(C)C]

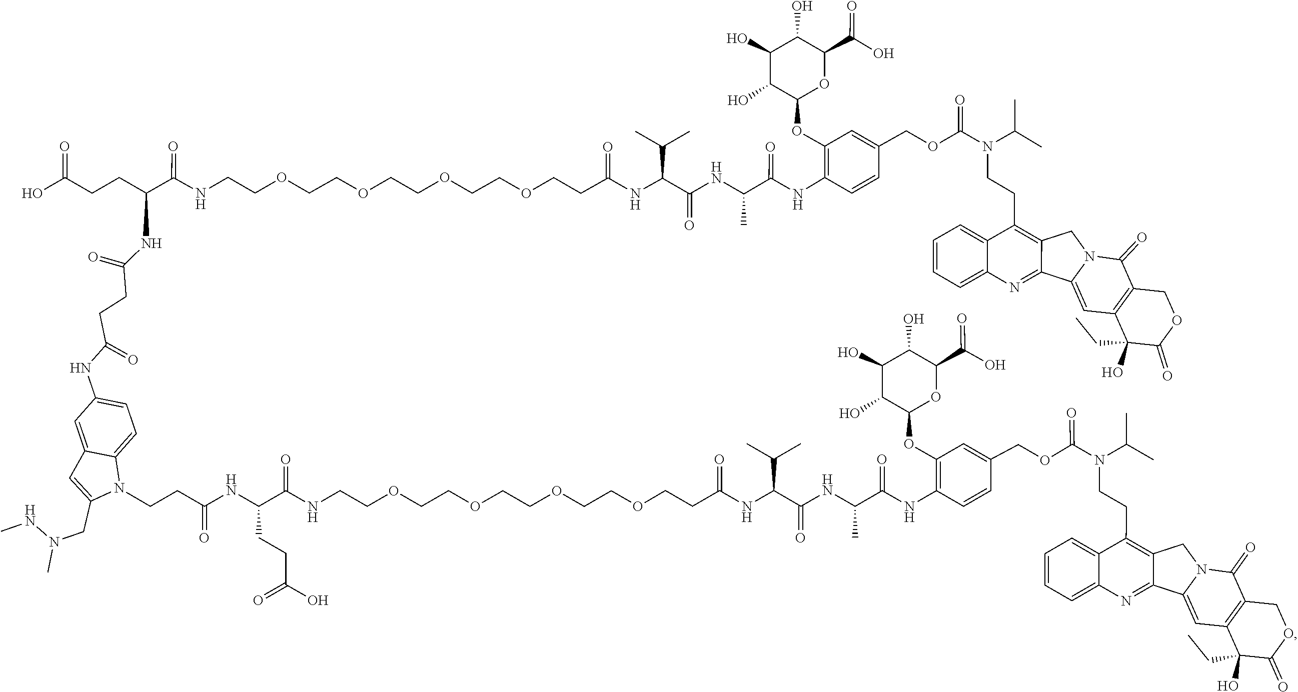 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCOCCOCCOCCOCCNC(=O)[C@H](CCC(=O)O)NC(=O)CCC(=O)Nc2ccc3c(c2)cc(CN(C)NC)n3CCC(=O)N[C@@H](CCC(=O)O)C(=O)NCCOCCOCCOCCOCCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)c(O[C@@H]2O[C@H](C(=O)O)[C@@H](O)[C@H](O)[C@H]2O)c1)C(C)C]

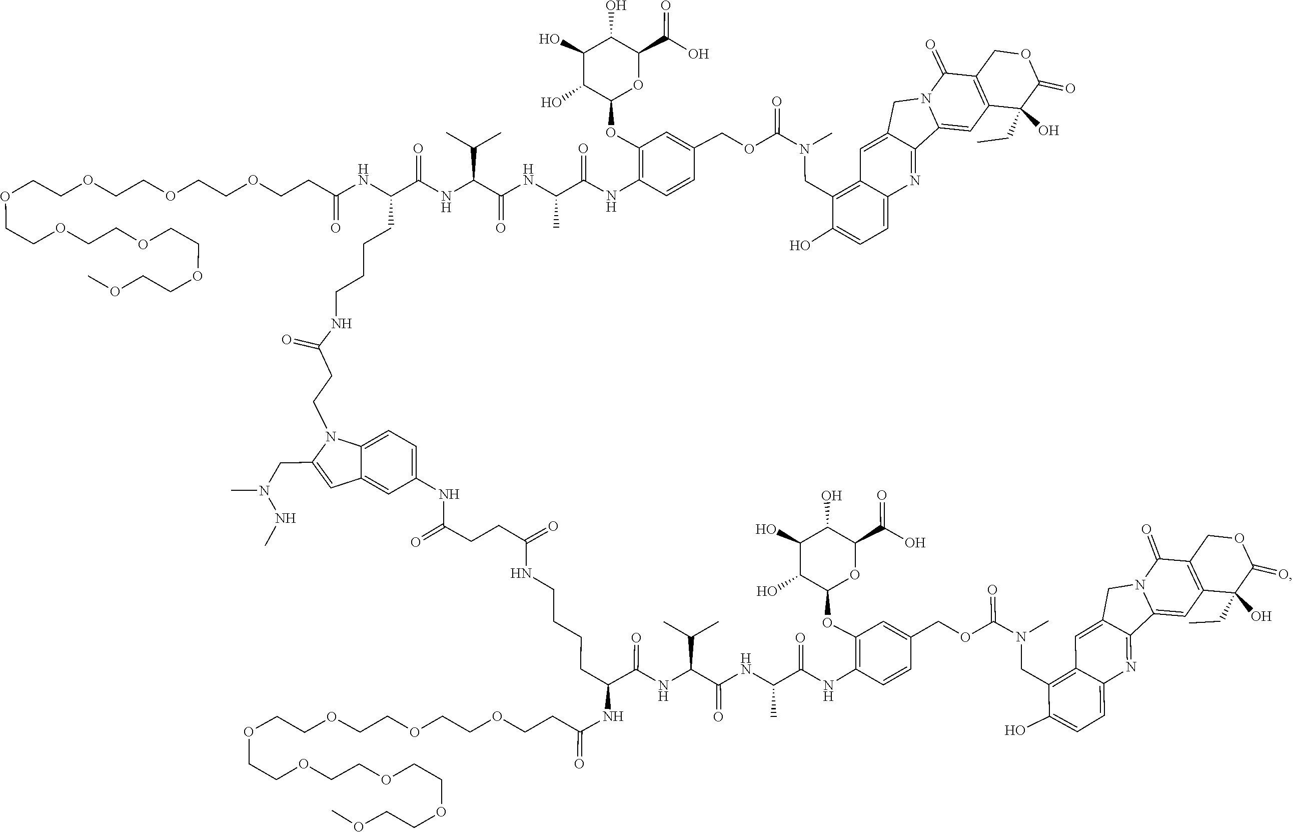 [SMILES: CC[C@@]1(O)C(=O)OCc2c1cc1n(c2=O)Cc2cc3c(CN(C)C(=O)OCc4ccc(NC(=O)[C@H](C)NC(=O)[C@@H](NC(=O)[C@H](CCCCNC(=O)CCC(=O)Nc5ccc6c(c5)cc(CN(C)NC)n6CCC(=O)NCCCC[C@H](NC(=O)CCOCCOCCOCCOCCOCCOCCOCCOC)C(=O)N[C@H](C(=O)N[C@@H](C)C(=O)Nc5ccc(COC(=O)N(C)Cc6c(O)ccc7nc8c(cc67)Cn6c-8cc7c(c6=O)COC(=O)[C@]7(O)CC)cc5O[C@@H]5O[C@H](C(=O)O)[C@@H](O)[C@H](O)[C@H]5O)C(C)C)NC(=O)CCOCCOCCOCCOCCOCCOCCOCCOC)C(C)C)c(O[C@@H]5O[C@H](C(=O)O)[C@@H](O)[C@H](O)[C@H]5O)c4)c(O)ccc3nc2-1]

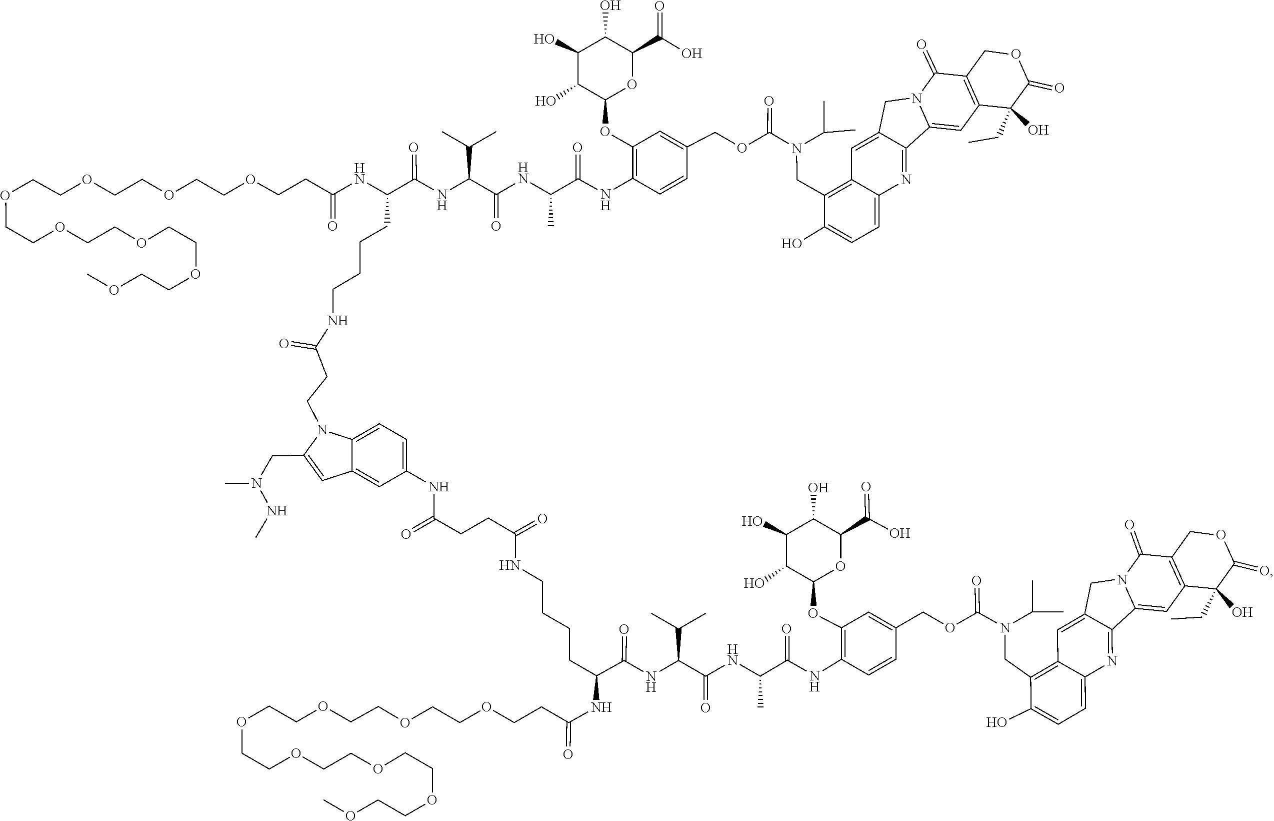 [SMILES: CC[C@@]1(O)C(=O)OCc2c1cc1n(c2=O)Cc2cc3c(CN(C(=O)OCc4ccc(NC(=O)[C@H](C)NC(=O)[C@@H](NC(=O)[C@H](CCCCNC(=O)CCC(=O)Nc5ccc6c(c5)cc(CN(C)NC)n6CCC(=O)NCCCC[C@H](NC(=O)CCOCCOCCOCCOCCOCCOCCOCCOC)C(=O)N[C@H](C(=O)N[C@@H](C)C(=O)Nc5ccc(COC(=O)N(Cc6c(O)ccc7nc8c(cc67)Cn6c-8cc7c(c6=O)COC(=O)[C@]7(O)CC)C(C)C)cc5O[C@@H]5O[C@H](C(=O)O)[C@@H](O)[C@H](O)[C@H]5O)C(C)C)NC(=O)CCOCCOCCOCCOCCOCCOCCOCCOC)C(C)C)c(O[C@@H]5O[C@H](C(=O)O)[C@@H](O)[C@H](O)[C@H]5O)c4)C(C)C)c(O)ccc3nc2-1]

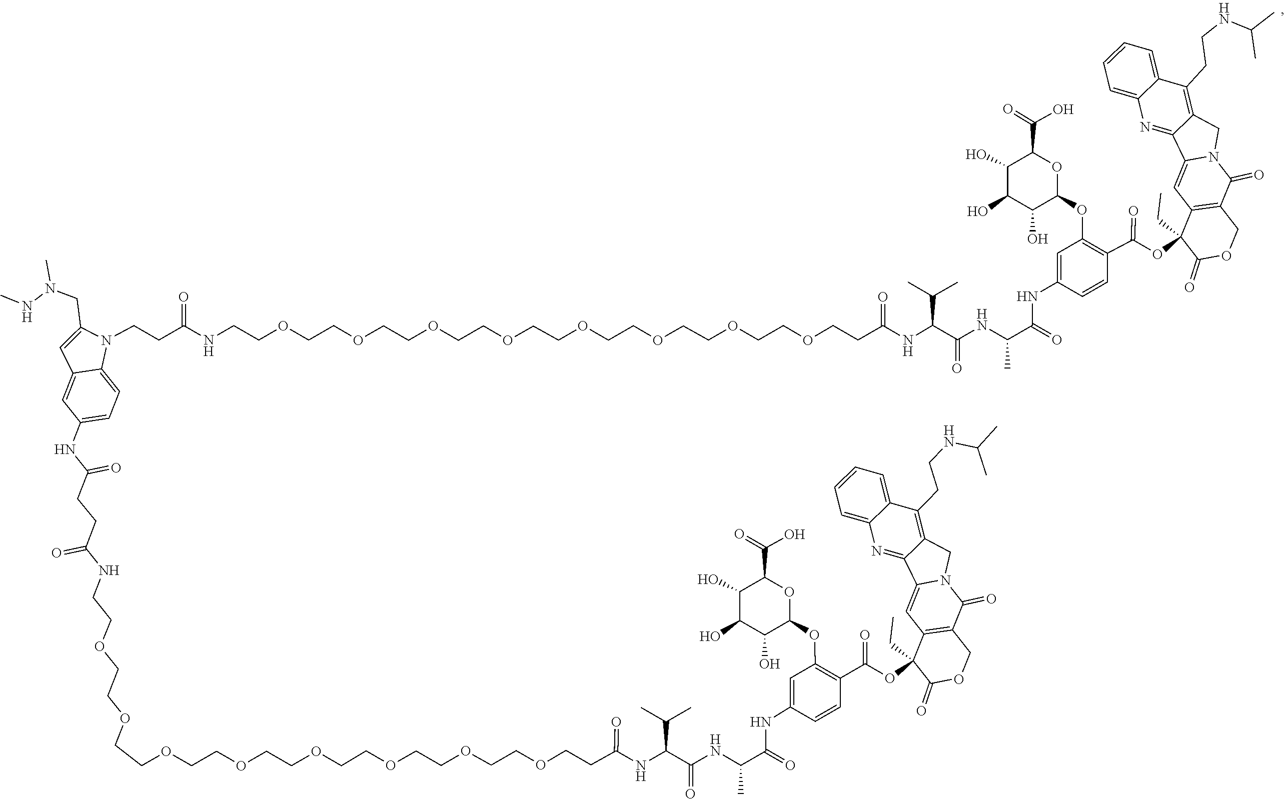 [SMILES: CC[C@@]1(OC(=O)c2ccc(NC(=O)[C@H](C)NC(=O)[C@@H](NC(=O)CCOCCOCCOCCOCCOCCOCCOCCOCCNC(=O)CCC(=O)Nc3ccc4c(c3)cc(CN(C)NC)n4CCC(=O)NCCOCCOCCOCCOCCOCCOCCOCCOCCC(=O)N[C@H](C(=O)N[C@@H](C)C(=O)Nc3ccc(C(=O)O[C@]4(CC)C(=O)OCc5c4cc4n(c5=O)Cc5c-4nc4ccccc4c5CCNC(C)C)c(O[C@@H]4O[C@H](C(=O)O)[C@@H](O)[C@H](O)[C@H]4O)c3)C(C)C)C(C)C)cc2O[C@@H]2O[C@H](C(=O)O)[C@@H](O)[C@H](O)[C@H]2O)C(=O)OCc2c1cc1n(c2=O)Cc2c-1nc1ccccc1c2CCNC(C)C]

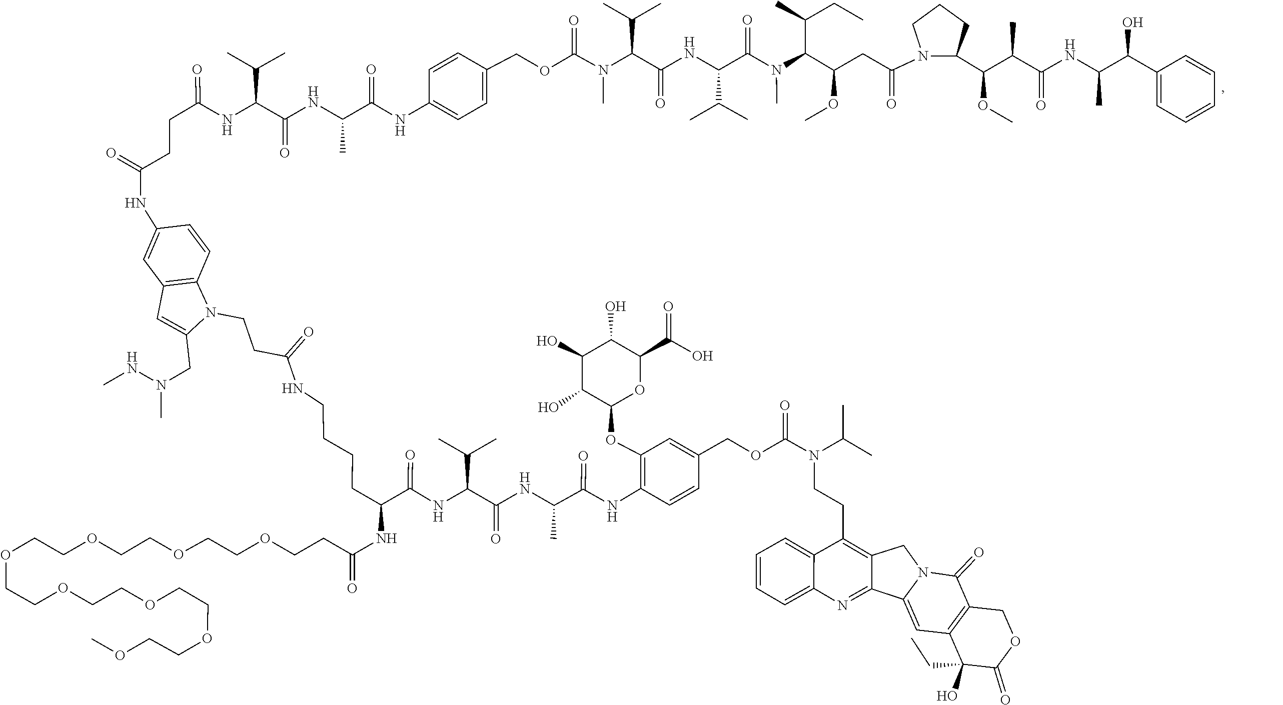 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCC(=O)Nc2ccc3c(c2)cc(CN(C)NC)n3CCC(=O)NCCCC[C@H](NC(=O)CCOCCOCCOCCOCCOCCOCCOCCOC)C(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)cc1)C(C)C]

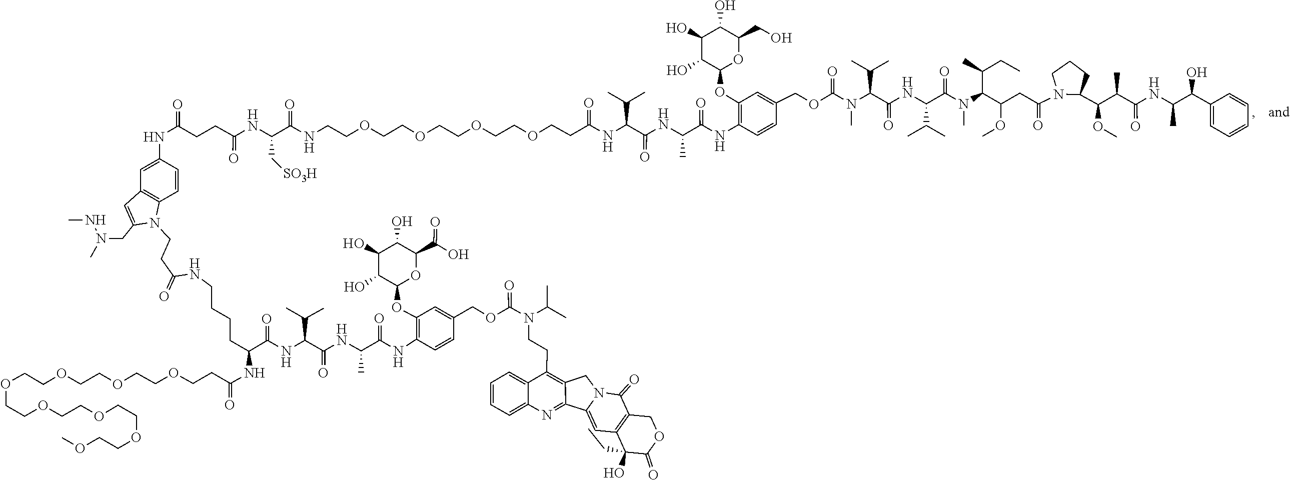

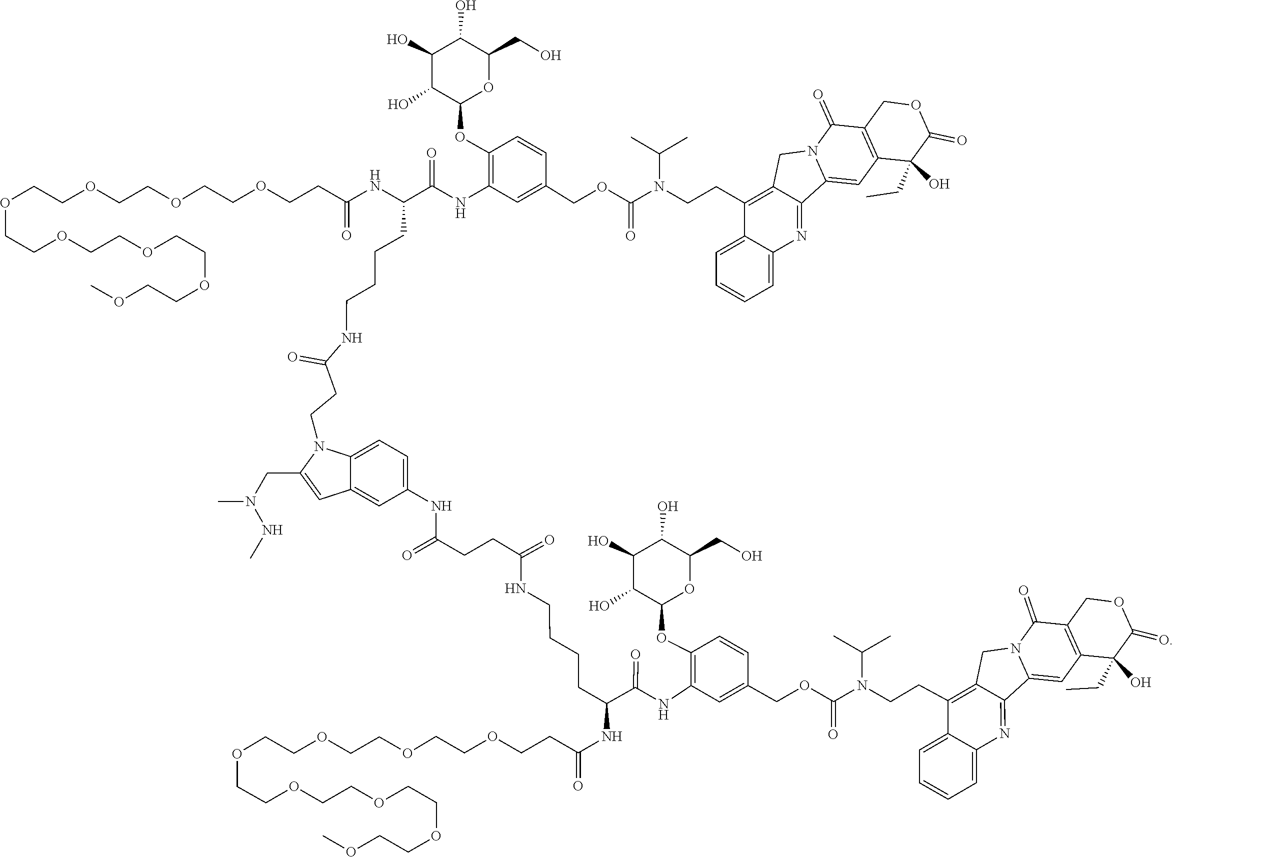 [SMILES: CC[C@@]1(O)C(=O)OCc2c1cc1n(c2=O)Cc2c-1nc1ccccc1c2CCN(C(=O)OCc1ccc(O[C@@H]2O[C@H](CO)[C@@H](O)[C@H](O)[C@H]2O)c(NC(=O)[C@H](CCCCNC(=O)CCC(=O)Nc2ccc3c(c2)cc(CN(C)NC)n3CCC(=O)NCCCC[C@H](NC(=O)CCOCCOCCOCCOCCOCCOCCOCCOC)C(=O)Nc2cc(COC(=O)N(CCc3c4c(nc5ccccc35)-c3cc5c(c(=O)n3C4)COC(=O)[C@]5(O)CC)C(C)C)ccc2O[C@@H]2O[C@H](CO)[C@@H](O)[C@H](O)[C@H]2O)NC(=O)CCOCCOCCOCCOCCOCCOCCOCCOC)c1)C(C)C]

34. The compound of any of claims 18-32, wherein the compound is selected from:

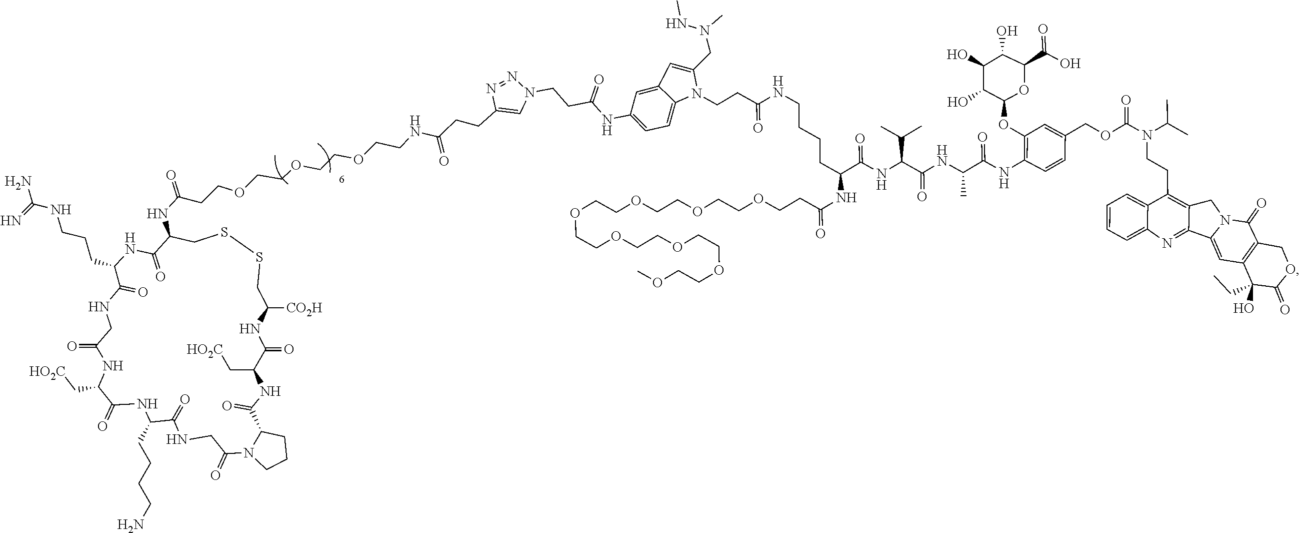

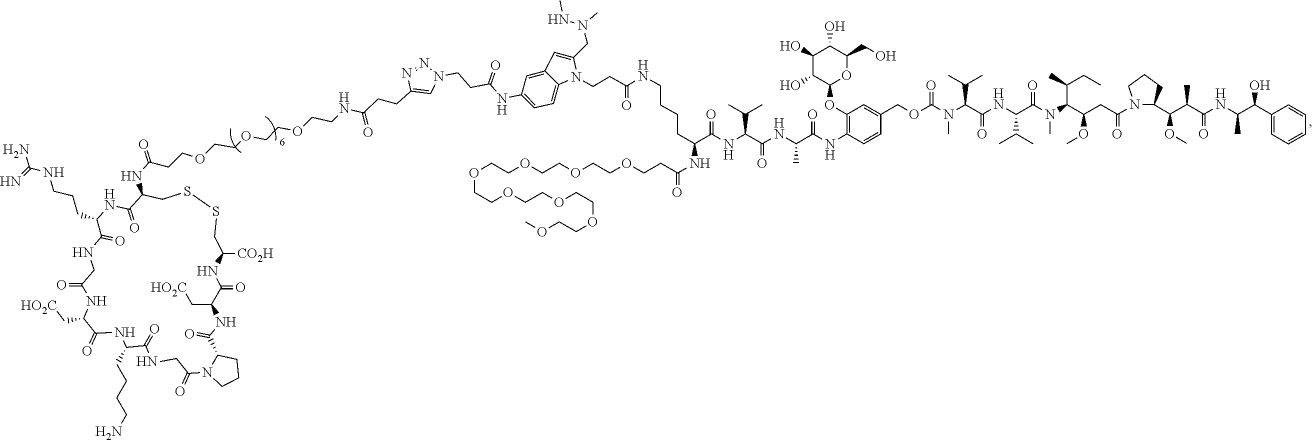

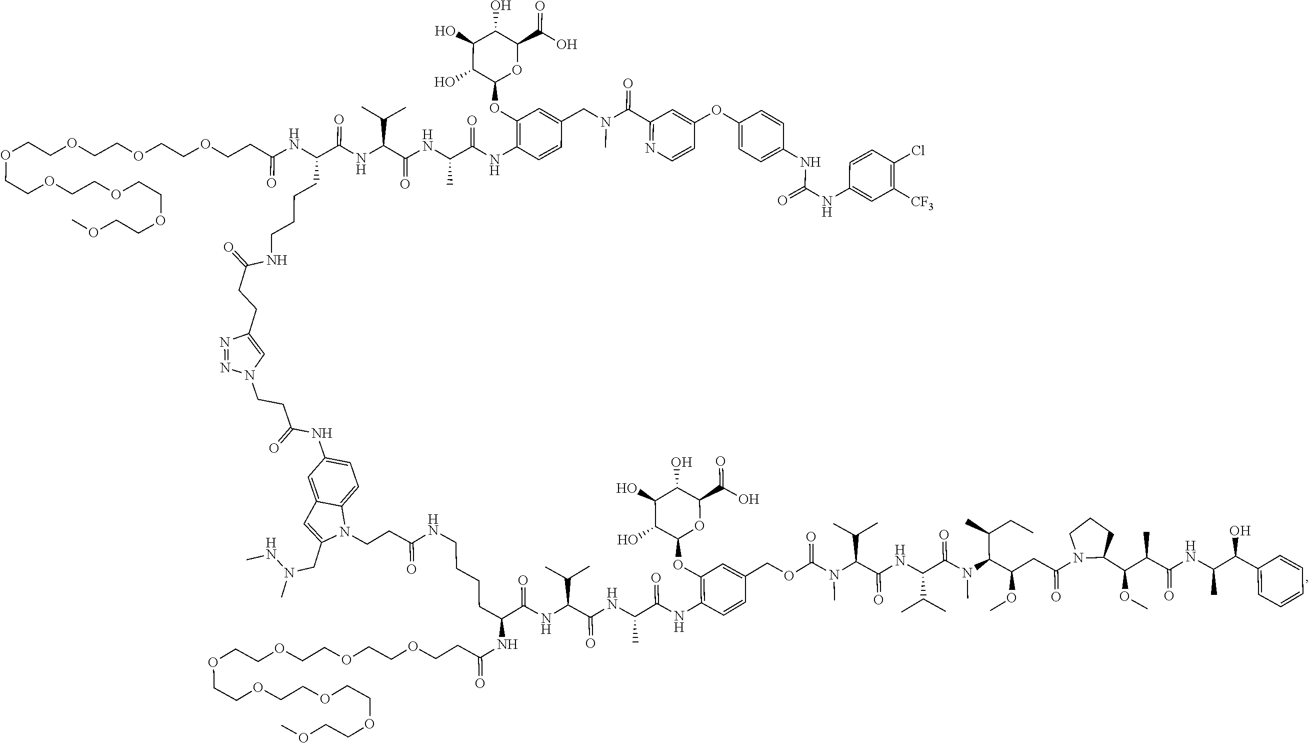

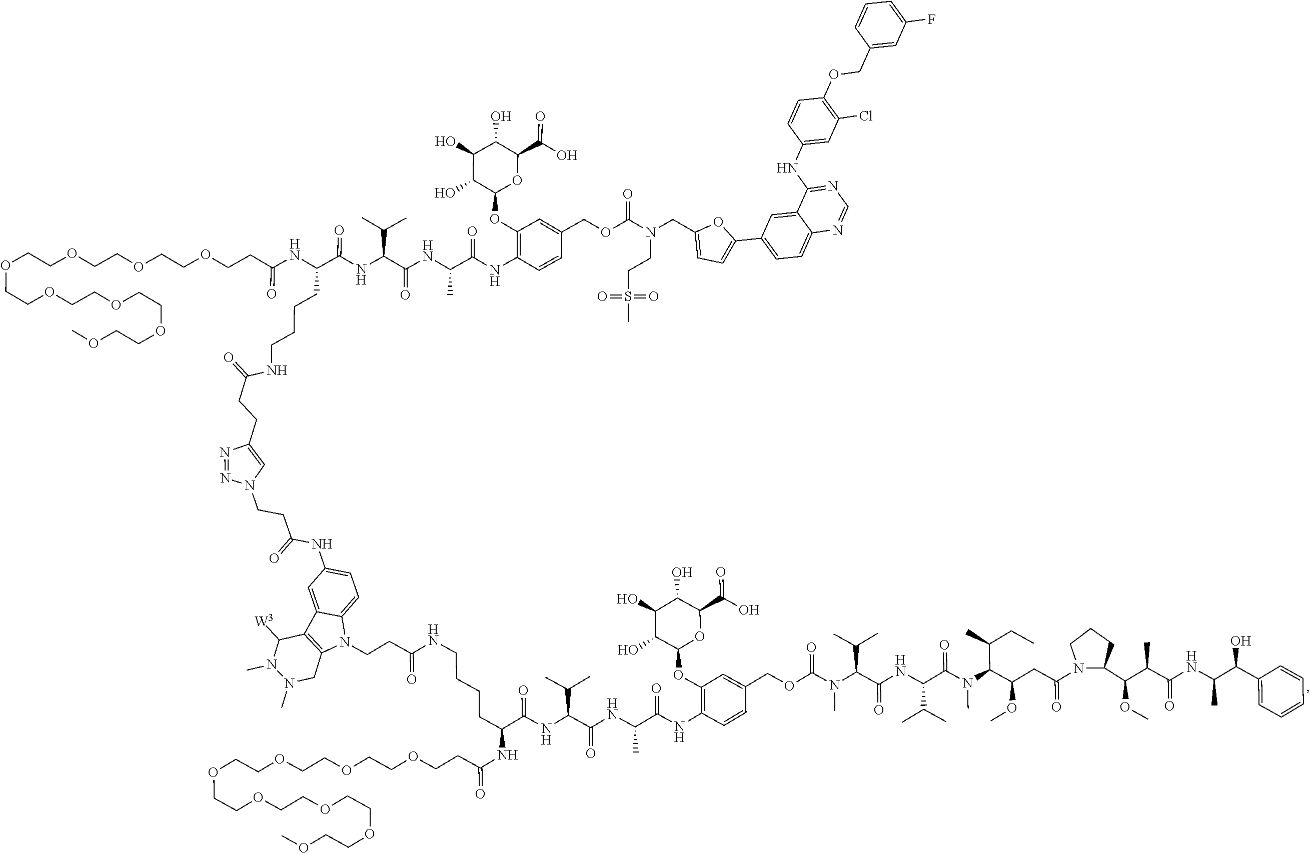

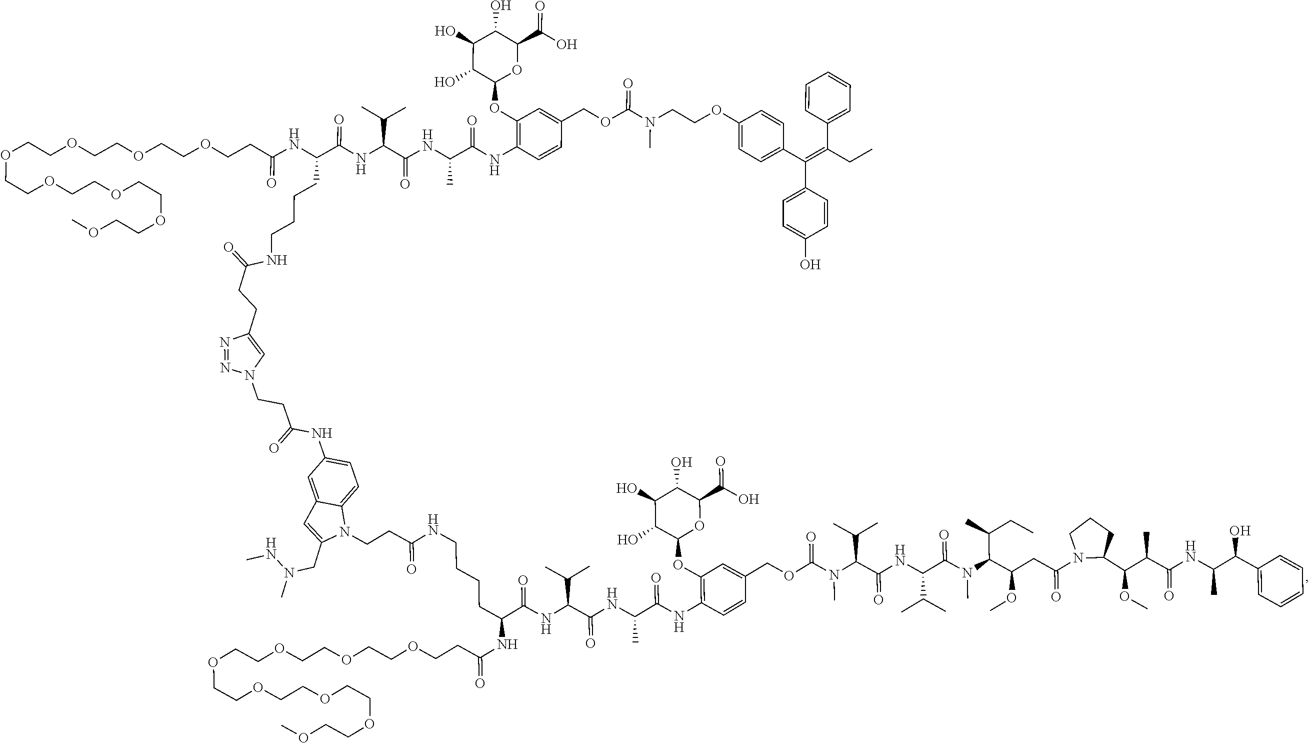

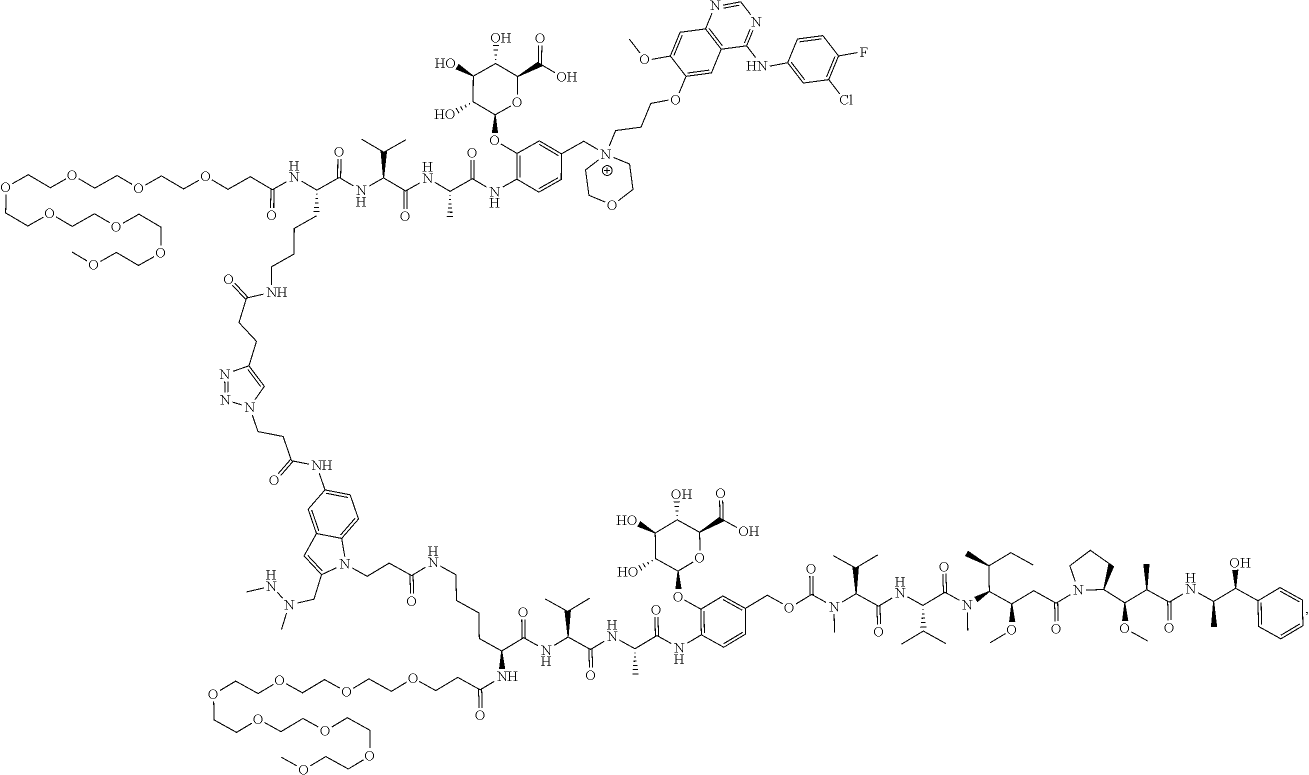

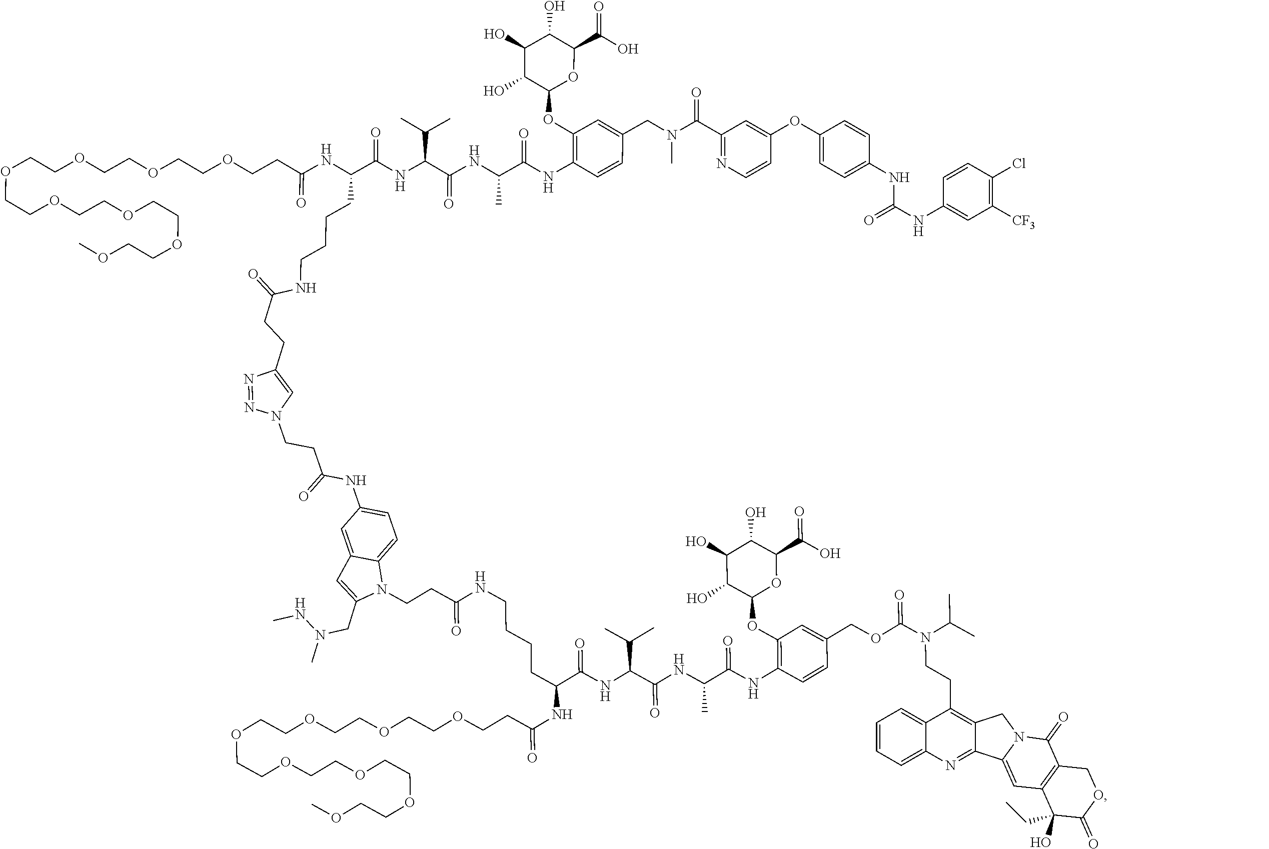 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CCCCNC(=O)CCn2c(CN(C)NC)cc3cc(NC(=O)CCn4cc(CCC(=O)NCCCC[C@H](NC(=O)CCOCCOCCOCCOCCOCCOCCOCCOC)C(=O)N[C@H](C(=O)N[C@@H](C)C(=O)Nc5ccc(CN(C)C(=O)c6cc(Oc7ccc(NC(=O)Nc8ccc(Cl)c(C(F)(F)F)c8)cc7)ccn6)cc5O[C@@H]5O[C@H](C(=O)O)[C@@H](O)[C@H](O)[C@H]5O)C(C)C)nn4)ccc32)NC(=O)CCOCCOCCOCCOCCOCCOCCOCCOC)C(C)C)c(O[C@@H]2O[C@H](C(=O)O)[C@@H](O)[C@H](O)[C@H]2O)c1)C(C)C]

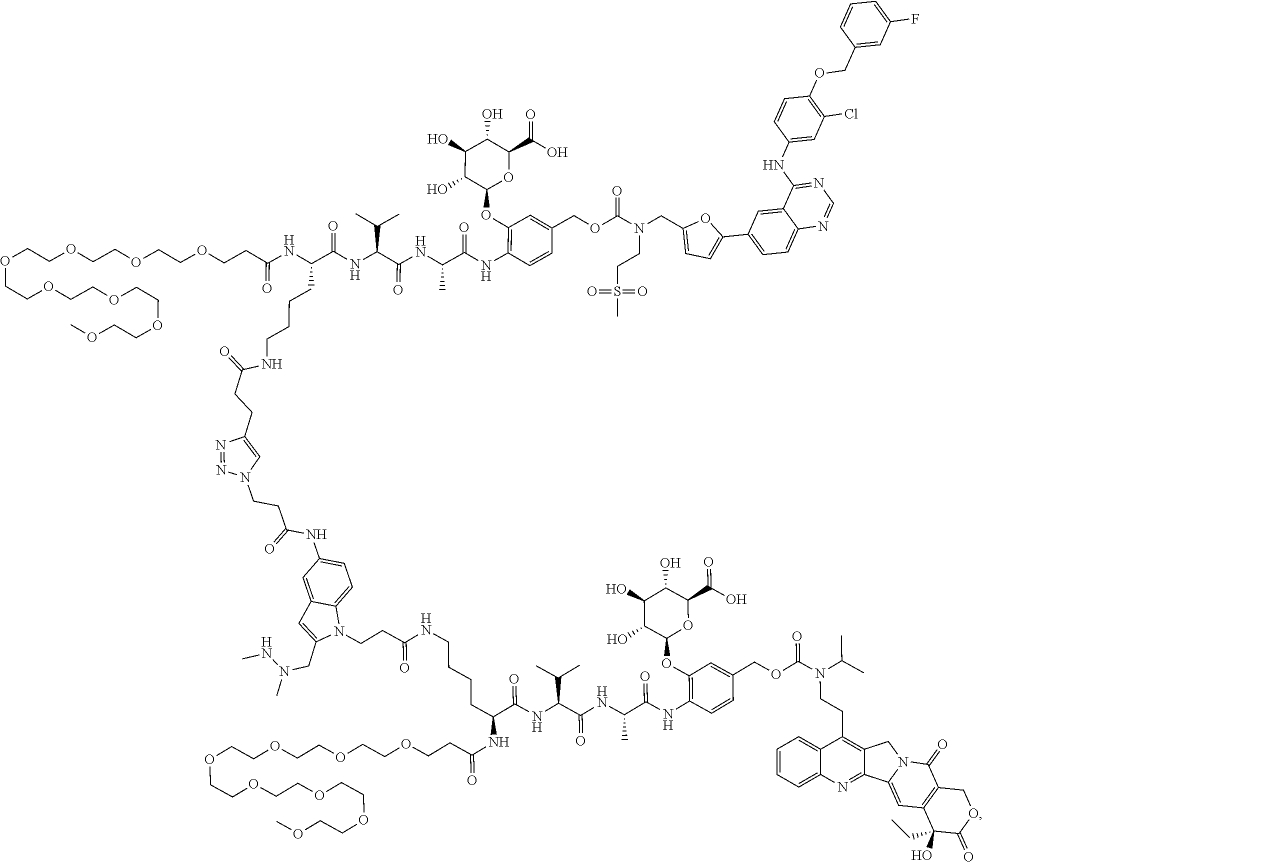 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CCCCNC(=O)CCn2c(CN(C)NC)cc3cc(NC(=O)CCn4cc(CCC(=O)NCCCC[C@H](NC(=O)CCOCCOCCOCCOCCOCCOCCOCCOC)C(=O)N[C@H](C(=O)N[C@@H](C)C(=O)Nc5ccc(COC(=O)N(CCS(C)(=O)=O)Cc6ccc(-c7ccc8ncnc(Nc9ccc(OCc%10cccc(F)c%10)c(Cl)c9)c8c7)o6)cc5O[C@@H]5O[C@H](C(=O)O)[C@@H](O)[C@H](O)[C@H]5O)C(C)C)nn4)ccc32)NC(=O)CCOCCOCCOCCOCCOCCOCCOCCOC)C(C)C)c(O[C@@H]2O[C@H](C(=O)O)[C@@H](O)[C@H](O)[C@H]2O)c1)C(C)C]

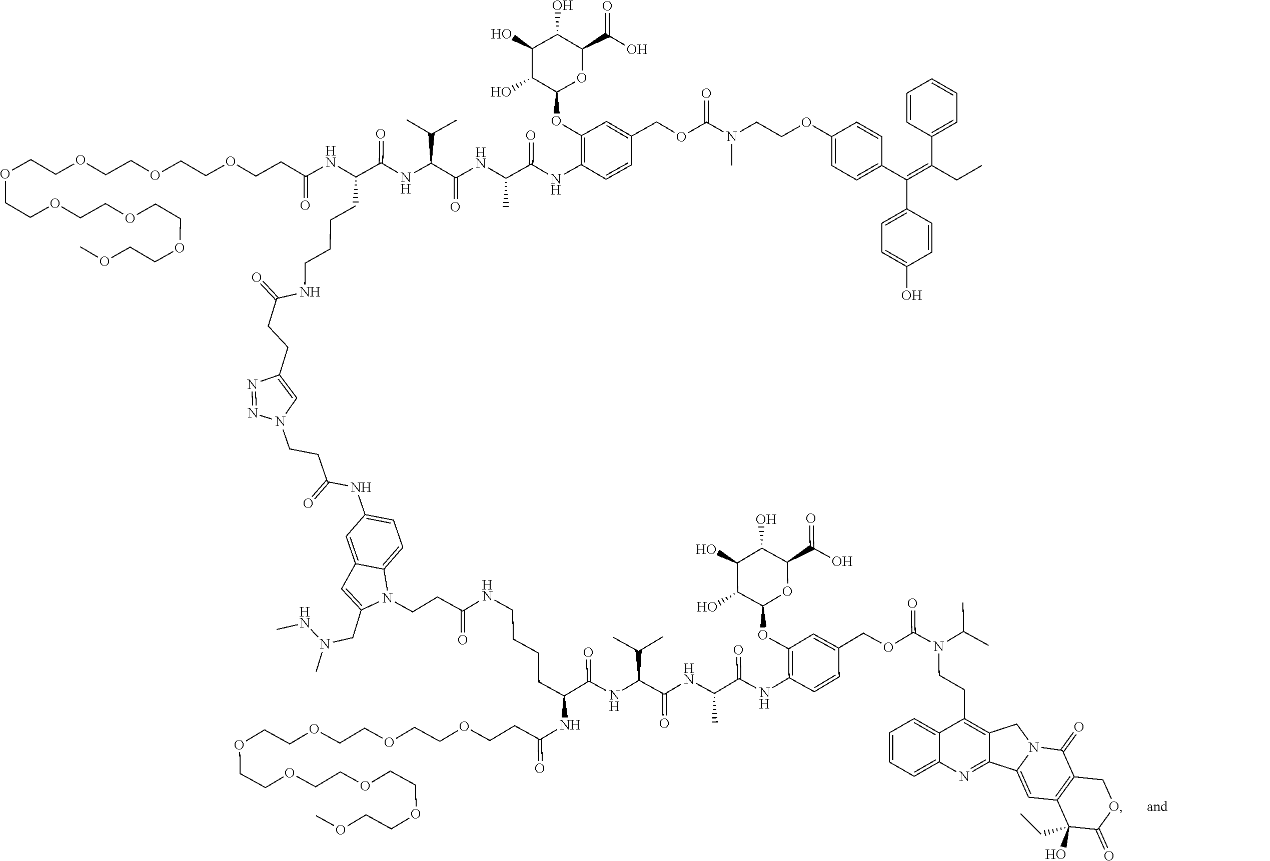 [SMILES: CC/C(=C(\c1ccc(O)cc1)c1ccc(OCCN(C)C(=O)OCc2ccc(NC(=O)[C@H](C)NC(=O)[C@@H](NC(=O)[C@H](CCCCNC(=O)CCc3cn(CCC(=O)Nc4ccc5c(c4)cc(CN(C)NC)n5CCC(=O)NCCCC[C@H](NC(=O)CCOCCOCCOCCOCCOCCOCCOCCOC)C(=O)N[C@H](C(=O)N[C@@H](C)C(=O)Nc4ccc(COC(=O)N(CCc5c6c(nc7ccccc57)-c5cc7c(c(=O)n5C6)COC(=O)[C@]7(O)CC)C(C)C)cc4O[C@@H]4O[C@H](C(=O)O)[C@@H](O)[C@H](O)[C@H]4O)C(C)C)nn3)NC(=O)CCOCCOCCOCCOCCOCCOCCOCCOC)C(C)C)c(O[C@@H]3O[C@H](C(=O)O)[C@@H](O)[C@H](O)[C@H]3O)c2)cc1)c1ccccc1 ; "and"]

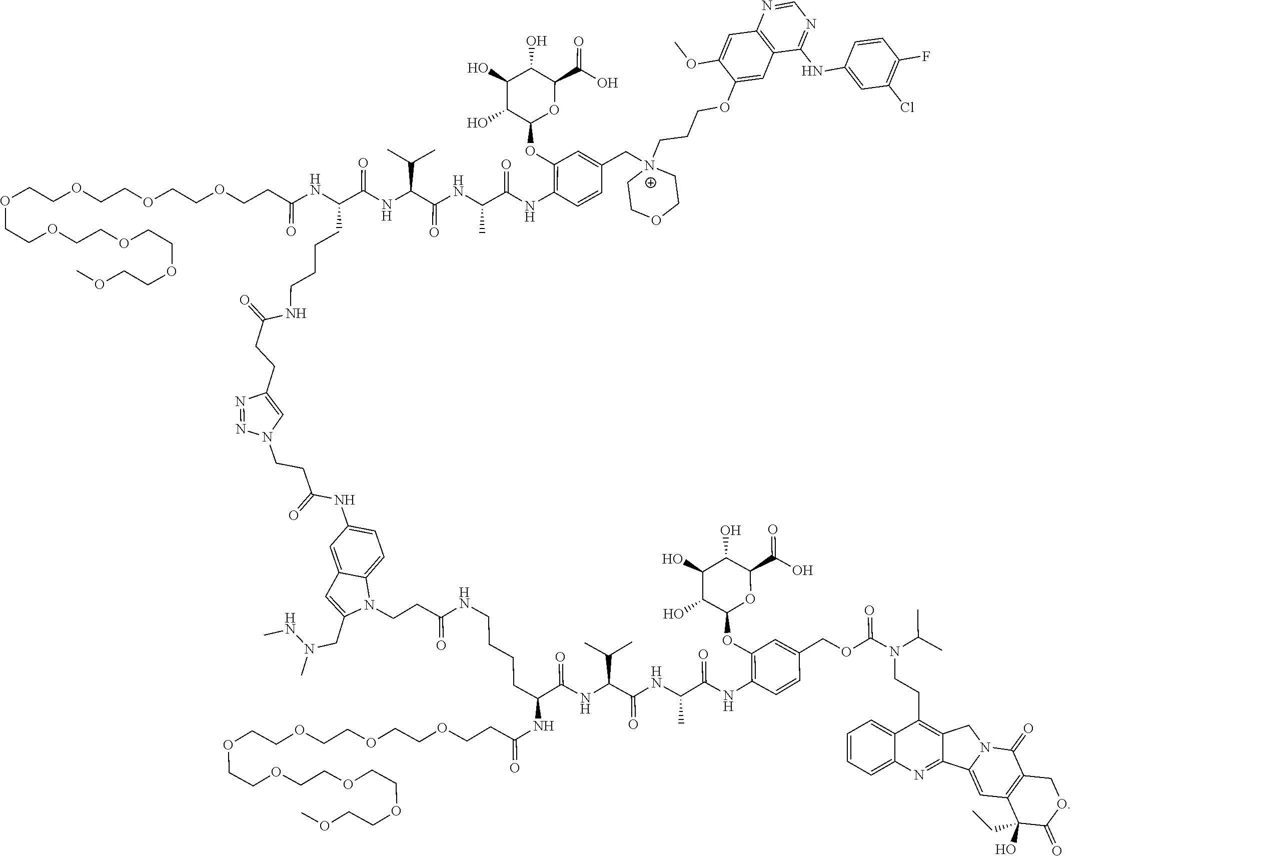 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CCCCNC(=O)CCn2c(CN(C)NC)cc3cc(NC(=O)CCn4cc(CCC(=O)NCCCC[C@H](NC(=O)CCOCCOCCOCCOCCOCCOCCOCCOC)C(=O)N[C@H](C(=O)N[C@@H](C)C(=O)Nc5ccc(C[N+]6(CCCOc7cc8c(Nc9ccc(F)c(Cl)c9)ncnc8cc7OC)CCOCC6)cc5O[C@@H]5O[C@H](C(=O)O)[C@@H](O)[C@H](O)[C@H]5O)C(C)C)nn4)ccc32)NC(=O)CCOCCOCCOCCOCCOCCOCCOCCOC)C(C)C)c(O[C@@H]2O[C@H](C(=O)O)[C@@H](O)[C@H](O)[C@H]2O)c1)C(C)C]

35. A pharmaceutical composition comprising:
a conjugate of any one of claims 1 to 17; and
a pharmaceutically-acceptable excipient.

36. A method comprising:
administering to a subject an effective amount of a conjugate of any one of claims 1 to 17.

37. A method of treating cancer comprising:
administering to a subject a therapeutically effective amount of a conjugate of any one of claims 1 to 17, wherein the administering is effective to treat the cancer in the subject.
